SEQUENCE LISTING (199 sequences)

SEQ ID NO: 1 - DNA, 1475 bp, unidentified
  misc_feature 1..1475: note = CYP76AD1
  source 1..1475: mol_type = unassigned DNA
aatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctca

SEQ ID NO: 2 - DNA, 825 bp, unidentified
  misc_feature 1..825: note = 4,5DOPA dioxygenase
  source 1..825: mol_type = unassigned DNA
atgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcc

SEQ ID NO: 3 - DNA, 1487 bp, unidentified
  misc_feature 1..1487: note = CycloDopa5-O-glucosyltransferase (cDOPA5GT)
  source 1..1487: mol_type = unassigned DNA
cgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttctc

SEQ ID NO: 4 - DNA, 1197 bp, unidentified
  misc_feature 1..1197: note = ADHa
  source 1..1197: mol_type = unassigned DNA
atgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggtag

SEQ ID NO: 5 - DNA, 828 bp, unidentified
  misc_feature 1..828: note = DODA
  source 1..828: mol_type = unassigned DNA
atgaaaatgatgaatggtgaggacgccaacgatcaaatgattaaagagtcgttctttatcacgcatggaaatccaattctcacggtagaggacacacaccctctgcgtccattctttgaaacttggagagaaaagatatttagtaagaaacccaaagctattctgatcattagtgggcattgggaaaccgttaagcctacagtgaatgcagttcatatcaatgacaccattcatgattttgacgattaccccgctgccatgtatcaatttaaataccctgctcccggagcacctgagcttgccaggaaagtggaggaaattttgaaaaagtccggctttgagacggccgaaaccgatgagaaaagaggtcttgatcatggcgcgtgggtcccccttatgctcatgtaccctgaagccgatataccagtgtgccaattaagtgttcagcctcaccttgatggaacctatcactataaccttggtagagcccttgcgcccttgaaaaacgatggggtcttaattatagggagtgggagcgcaacgcaccccttggatgagactcctcattattttgatggcgttgctccttgggccgcagcttttgactcttggttacgcaaggccttgattaatgggcggttcgaagaggtgaacatatatgaaactaaagctccaaactggaaattggctcacccctttcctgagcacttttatcccctccatgtagtgctgggtgctgcgggggagaagtggaaagctgagcttatacattcctcatgggaccacggcaccttgtgccacggttcatacaaattcacaagcgcttaa

SEQ ID NO: 6 - DNA, 1494 bp, unidentified
  misc_feature 1..1494: note = CYP76AD1 (3)
  source 1..1494: mol_type = unassigned DNA
atggaccatgcgacccttgcaatgatactagccatttggttcatcagttttcacttcattaaactactctttagccagcaaaccactaaactcctaccaccgggtcccaaaccattgccaatcataggaaacatccttgaggtgggaaagaaaccacaccggtcgttcgctaatcttgctaaaattcacgggccattaattagtcttagactcgggtcagtgactacaatagttgtgagctctgccgatgttgccaaagaaatgtttctaaagaaagatcatcctctaagtaatcgtaccatacccaacagcgttactgcgggtgatcatcacaaactaaccatgtcgtggttaccagtgtctcccaagtggaggaattttcggaaaataaccgctgtgcacttgctcagtccccaacggctcgacgcatgtcaaacatttagacatgccaaagtccaacagctatacgaatatgttcaagaatgcgctcagaagggtcaagcagtagacatcggtaaggcagctttcactacaagtcttaacctactttcaaaattatttttctcagtcgagttggctcaccataaaagtcagacatcacaagaattcaaggagctaatctggaacataatggaagatatcggtaaacccaactatgcagattattttcctatattgggctgcgtcgaccccagtggtatacggagacgattggcatgttcatttgacaaactcattgctgtttttcagtctataatttgcgaaagactagctccggatagttccacagcaactacaaccacaactgacgatgtgcttgatgtgttgctgcagttgttcaagcaaaatgagttaacaatgggtgagattaatcaccttctggtggatatattcgacgccggtactgatactacatcgtctacttttgagtgggtgatggctgagcttatcaggaacccagaaatgatggaaaaagcacaagaagagataaaacaagtgttggggaaagataaacaaatccaagagagtgatataatcaacttgccttatctgcaagcaataatcaaggagacactacgtctacaccctcccacagtctttctcttaccacggaaagccgacaccgacgtggaactgtacggatatatagttccgaaagatgctcaaattttagtcaacctttgggccattgggcgcgatcccaatgcctggcagaatgccgatatttttagtccagagagatttatcgggtgtgaaatcgatgttaaaggtagagacttcggactcttacccttcggggcaggcagaaggatatgtccaggaatgaacttagccatccgaatgctaacactgatgctagcaacccttctccaattctttaattggaagctagaaggtgacatctcgcccaaagaccttgacatggacgagaaatttggaattgctcttcaaaaaacaaaacccttaaaacttatacccattcccagatattaa

SEQ ID NO: 7 - DNA, 1503 bp, unidentified
  misc_feature 1..1503: note = cDOPA5GT
  source 1..1503: mol_type = unassigned DNA
atgactgcaatcaaaatgaatacgaatggtgagggtgaaacccaacatatacttatgataccatttatggcccaagggcaccttcgaccgtttctggaattggccatgttcttatacaaacgatcacatgtcatcataactctgctaacaactccccttaacgctgggttcctgagacatctgttgcatcaccattcctacagctcctctgggatacggatcgtagaattgccgtttaattcgactaatcacgggctcccacccggtatagaaaatacagacaaattaacactgcctttagtcgtatccttgtttcattctacaatttccctagacccccacctacgcgattatatttcgagacacttcagtcctgcacgacccccactgtgtgttattcacgatgtttttctcggttgggtggaccaagtagctaaggatgtagggtccacaggcgtcgtttttacgaccggcggggcttatggcacctctgcatatgtatctatatggaatgacttaccacaccaaaattactccgacgatcaggagtttccattgccaggattccccgagaatcataaatttcgaagatctcaactgcatcgtttccttaggtatgctgatgggtctgatgactggtctaaatatttccaaccccaactacggcaaagtatgaaaagttttgggtggctatgcaatagtgtggaagagattgaaacattgggcttctcgattctaagaaactacaccaagcttccaatctggggcataggcccacttatagcctccccggtacagcacagttcaagtgacaataactctactggagcagagtttgtgcagtggttgagtcttaaagaacccgacagtgtattatatatatctttcgggagtcaaaatactatttcaccaactcagatgatggaattggctgcagggttggagtcgagtgagaaaccgtttctgtgggttatacgtgccccatttgggtttgatattaatgaggaaatgcgtcccgaatggcttcccgaggggtttgaagagaggatgaaagtcaaaaagcaagggaagcttgtttacaaattggggcctcaattggaaatccttaatcacgaaagtatcgggggttttctaacgcattgcgggtggaactctattcttgagtccctaagagaaggtgttcccatgttgggatggccactagcagctgaacaagcttacaacctaaaatatctagaggatgaaatgggtgtagcagtcgaactagccagaggcctcgagggggaaatttccaaagaaaaggttaagagaatagttgagatgatcttagagcggaatgaaggttcaaaaggatgggagatgaaaaacagggcagtggaaatggggaaaaagttaaaagatgccgtcaacgaggaaaaagagttgaaaggttcttcagtcaaagcaatagacgatttccttgacgctgtgatgcaagcaaagctcgaaccgtcattgcaatga

SEQ ID NO: 8 - protein, 398 aa, unidentified
  REGION 1..398: note = ADHalpha
MISLSSFHPSSTTATATAAAATTHPPQQCPAFSSPPSHLSLPLRHPRQHLVVRCGGGGSASESVFNRDSAATRVSNDHLDVSKRDVKLKIAIIGFGNFGQFLAKTMAKQGHRVLAYSRSDYSRAAKEIGVEYFTDADDLCEEHPEVILLCTSILSTEKVLRSLPLHRLRRSTLFADVLSVKEFPRSLFLQLLPKDFDILCTHPMFGPDSGKDGWGGLPFVFDKVRVGSDQSRTSRAEAFLDVFRNAGCRMVEMSCVDHDKHAAGSQFITHMMGRVLEKLALENTPINTKGYESLLNLVDNTARDSFELFYGLFLYNKNAMEQLDRMDWAFEMVKKQLSGYLHDLVRKQLMLEGNNDQAEVTFDKPLMLPSPTINPPQIVPSADMAEKKHDLVVVNGTR

SEQ ID NO: 9 - protein, 275 aa, unidentified
  REGION 1..275: note = DODA
MKMMNGEDANDQMIKESFFITHGNPILTVEDTHPLRPFFETWREKIFSKKPKAILIISGHWETVKPTVNAVHINDTIHDFDDYPAAMYQFKYPAPGAPELARKVEEILKKSGFETAETDEKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYETKAPNWKLAHPFPEHFYPLHVVLGAAGEKWKAELIHSSWDHGTLCHGSYKFTSA

SEQ ID NO: 10 - protein, 497 aa, unidentified
  REGION 1..497: note = CYP76AD1
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSQTSQEFKELIWNIMEDIGKPNYADYFPILGCVDPSGIRRRLACSFDKLIAVFQSIICERLAPDSSTATTTTTDDVLDVLLQLFKQNELTMGEINHLLVDIFDAGTDTTSSTFEWVMAELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 11 - protein, 500 aa, unidentified
  REGION 1..500: note = cDOPA5GT
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 12 - protein, 497 aa, unidentified
  REGION 1..497: note = CYP76AD1 (2)
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 13 - protein, 275 aa, unidentified
  REGION 1..275: note = DODA (2)
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 14 - protein, 500 aa, unidentified; identical to SEQ ID NO: 11
  REGION 1..500: note = DOPA

SEQ ID NO: 15 - protein, 497 aa, unidentified; identical to SEQ ID NO: 10
  REGION 1..497: note = BvCYP76AD1

SEQ ID NO: 16 - protein, 497 aa, unidentified
  REGION 1..497: note = BvCYP76AD1 W13L
MDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSQTSQEFKELIWNIMEDIGKPNYADYFPILGCVDPSGIRRRLACSFDKLIAVFQSIICERLAPDSSTATTTTTDDVLDVLLQLFKQNELTMGEINHLLVDIFDAGTDTTSSTFEWVMAELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 17 - protein, 275 aa, unidentified; identical to SEQ ID NO: 9
  REGION 1..275: note = BvDODA

SEQ ID NO: 18 - protein, 500 aa, unidentified; identical to SEQ ID NO: 11
  REGION 1..500: note = MjDOPA5GT

SEQ ID NO: 19 - protein, 495 aa, unidentified
  REGION 1..495: note = AmCYP76AD1
MDNATLAMILTIWLISINFIKMFFTHQNTKLILPPGPKPLPIIGNILEVGKKPHRSFANLAKIHGPLISLRLGSVTTIVVSSAEVAKEMFLKKDQPLSNRNVPNSVTAGDHHKLTMSWLPVSPKWRNFRKITAVHLLSPLRLDACQSLRQAKVQQLYQYVQECAQKGQSIDIGKAAFTTSLNLLSKLFFSKELACHKSHESQEFKQLIWNIMEDIGKPNYADYFPILGCIDPLGIRRRLAANFDKLIAVFQTIISERLENNKNANATNDVLDVLLQLYKQKELSMGEINHLLVDIFDAGTDTTSSTFEWVMTELIRNPNMMEKAQQEIQEVLGKDRQIQESDIIKLPYLQALIKETLRLHPPTVFLLPRKADMDVELYGYVVPKDAQILVNLWAIGRDPQAWEKPNAFLPERFLGSDVDVKGRDFGLLPFGAGKRICPGMNLAIRMLTLMLATLLQFFDWKLEEGMNPQDLDMDEKFGIALQKTKPLEIIPSRRH

SEQ ID NO: 20 - protein, 269 aa, unidentified
  REGION 1..269: note = AmDODA
MVEKEMNFNETFFITHGNPILTVEDSHPLRPFFETWKQNIFSKKPKAILVISGHWETDFPSVNAVDINDTIHDFDDYPDAMYQLKYPAPGSIEMAKKVEEILTKSGYKTINMDKKRGLDHGAWVPLMYMYPQADIPVCQLSVQPKLSGTYHYNLGRALAPLKQEGVLIIGSGSATHPLDETPHFHNGVAPWAHEFHSWLHSALINRRIEEVNTYESKAPNWELAHPFPEHFYPLHIVMGAAGDNWKAELIHSSWDHGTLYHGAYKFTSN

SEQ ID NO: 21 - protein, 478 aa, unidentified
  REGION 1..478: note = AmDOPA
MDSSKTKHIILLPFLAQGHLRPFLHLANFLRSHTPFTLSILTTPLNAANLRRQSNNINIYDLPFNSTDHNLPPNTENTEKLPLTSIISLFYASTSLQPHVRNHLTRHHLDNPPICIIFDIFLGWADNLARSIGSTGVCFNTGGAYGLAAYMSIWMHLPHRNVPDNVEFSLPEFPENRKFKRNQLHRFLRFADGTDDWSGFFQPQIKYSRNCSAWLCNSIEEIEPLGFEVLRAMLKVPVWGIGPLVKTENSCDEDDEQGCVEWLNQFEKGSVLYISFGSQNTVTPIQMMELAKGLEESGAKFLWVIRPPFGFDINGEFKPEWLPEGFEKRVMERKQGKLVKKWGPQMEILRNKATGAFLSHCGWNSLIEALSEGVPIIGWPLAAEQAYNSKMLVEEMGVAVELTRGLEGELSSEGVKKVVEMVMDREEGSFGCEMKKAAVVIGQKLRDAMKVEGDYRGCSLRSLDEFVEFIVSRKATLS

SEQ ID NO: 22 - protein, 497 aa, unidentified
  REGION 1..497: note = CqCYP76AD1
MDHATLAMILAIWFVVFHFIKMLFTSQTTKLLPPGPKPLPLIGNILEVGEKPHQSFANLAKIHGPLISLRLGSVTTIVVSSAEVAKEMFLKKDHPLSNRTVPNSVTAGDHHKLTMSWLPVSPKWRNFRKITAVHLLSPQRLDACQTLRHAKVQQLFQYVQECAQKGQAVDIGKAAFTTSLNLLSKLFFSVELAHHKSHTSQQFKELIWNIMEDIGKPNYADYFPILGCLDPSGIRRRLASNFDKLIAVFQSIICQRIGNGQDSASTKTTDDVLDILLDLHKQKELSMGEINHLLVDIFDAGTDTTSSTFEWVMAELIRNPKMMEKAQEEIEQVLGKDRQIQESDIIKLPYLQAIIKETLRLHPPTVFLLPRKADSDVELYGYVVPKDAQILVNLWAIGRDPQAWVKPDVFLPERFLGSEIDVKGRDFGLLPFGAGRRICPGMNLAIRMLTLMLATLLQFFNWKLEEGMKAEDLDMDEKFGIALQKTKPLQIIPVLRY

SEQ ID NO: 23 - protein, 497 aa, unidentified
  REGION 1..497: note = CqCYP75AD1 W13L
MDHATLAMILAILFVVFHFIKMLFTSQTTKLLPPGPKPLPLIGNILEVGEKPHQSFANLAKIHGPLISLRLGSVTTIVVSSAEVAKEMFLKKDHPLSNRTVPNSVTAGDHHKLTMSWLPVSPKWRNFRKITAVHLLSPQRLDACQTLRHAKVQQLFQYVQECAQKGQAVDIGKAAFTTSLNLLSKLFFSVELAHHKSHTSQQFKELIWNIMEDIGKPNYADYFPILGCLDPSGIRRRLASNFDKLIAVFQSIICQRIGNGQDSASTKTTDDVLDILLDLHKQKELSMGEINHLLVDIFDAGTDTTSSTFEWVMAELIRNPKMMEKAQEEIEQVLGKDRQIQESDIIKLPYLQAIIKETLRLHPPTVFLLPRKADSDVELYGYVVPKDAQILVNLWAIGRDPQAWVKPDVFLPERFLGSEIDVKGRDFGLLPFGAGRRICPGMNLAIRMLTLMLATLLQFFNWKLEEGMKAEDLDMDEKFGIALQKTKPLQIIPVLRY

SEQ ID NO: 24 - protein, 272 aa, unidentified
  REGION 1..272: note = CqDODA
MKMSGEDNSMIKETFFITHGNPILTVEDTHPLRPFFETWREKIFTMKPKAILVISGHWETDHPAVNAVHINDTIYDFDDYPQAMYKLKYPAPGSPDLASRVEEIMKKSGFDTVHIDKKRGLDHGAWVPLMYMYPEADIPVCQLSVQPKLDGTYHYNLGRALAPLRDEGVLIIGSGSATHPLDETPHYHGGVAPWAADFDSWLDVALTKGRFEEVNTYETKAPNWELAHPFPEHFYPLHVVVGAAGEKWKAELIHTSWDHGTLCHGSYKFTST

SEQ ID NO: 25 - protein, 491 aa, unidentified
  REGION 1..491: note = CqUDP92A1 L79V
MESSTKEQEQQHIILLPFLAQGHLRPFLHLAHRLLSLTPFNLSLLTTPLNAANLRRQSDNFNINLNIIELPFTSTDHGVPPNTENTDKLSLTSIIKLFHASTSLEPHVRDYLTRHHLHDPPVCIIFDVFLGWADNVARSVGSTGICFNTGGAYGVGAYVSIWSNLPHRNVGDDEEFSLAGFPEDRKLRRNQLHRFLRFADGSDEWSRFFQPQIKSSLNCSGWLCNSIEEIEPLGFQVFRNFTKSPIWGIGPLIMTSSKKDDDEKEEACLRWLNQFENDSVLYICFGSQNTVTPIQMMELAKGLEESKIPFLWVIRPPFGFDFNGEFKPEWLPEKFEERMMEKKQGMLVRDWVPQLDILRHEATGGFLSHCGWNSVLEGLREGVPILGWPLAAEQAYNSKMMVEEMGVAVELTRGLEGEVKKEWVKSVVEMVLDRKEGSCGWEMKKKAVEIGEKLKDAWKIEGDYKGSSVKAMDNFVEFIVSCRGKKSKEDA

SEQ ID NO: 26 - DNA, 1992 bp, Zea mays
  misc_feature 1..1992: note = Exemplary Zea mays Ubiquitin 1 promoter (ZmUbi1)
  source 1..1992: mol_type = unassigned DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 27 - DNA, 1400 bp, Oryza sativa
  misc_feature 1..1400: note = Exemplary Oryza sativa Actin 1 promoter (OsAc1)
  source 1..1400: mol_type = unassigned DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcgctctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtaga

SEQ ID NO: 28 - DNA, 1861 bp, Panicum virgatum
  misc_feature 1..1861: note = Exemplary Panicum virgatum L. Ubiquitin 2 promoter (PvUbi2)
  source 1..1861: mol_type = unassigned DNA
gaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcag

SEQ ID NO: 29 - DNA, 2000 bp, Panicum virgatum
  misc_feature 1..2000: note = Exemplary Panicum virgatum L. Ubiquitin 1 fusion promoter (PvUbi1+3)
  source 1..2000: mol_type = unassigned DNA
ccactggagaggggcacacacgtcagtgtttggtttccactagcacgagtagcgcaatcagaaaattttcaatgcatgaagtactaaacgaagtttatttagaaatttttttaagaaatgagtgtaattttttgcgacgaatttaatgacaataattaatcgatgattgcctacagtaatgctacagtaaccaacctctaatcatgcgtcgaatgcgtcattagattcgtctcgcaaaatagcacaagaattatgaaattaattttacaaactatttttatttaatactaataattaactgtcaaagtttgtgctactcgcaagagtagcgcgaaccaaacacggcctggaggagcacggtaacggcgtcgacaaactaacggccaccacccgccaacgcaaaggagacggatgagagttgacttcttgacggttctccacccctctgtctctctgtcactgggccctgggtccccctctcgaaagttcctctggccgaaattgcgcggcggagacgaggcgggcggaaccgtcacggcagaggattccttccccaccctgcctggcccggccatatataaacagccaccgcccctccccgttccccatcgcgtctcgtctcgtgttgttcccagaacacaaccaaaatccaaatcctcctcctcctcccgagcctcgtcgatccctcacccgcttcaaggtacggcgatcctcctctcccttctcccctcgatcgattatgcgtgttccgtttccgtttccgatcgagcgaatcgatggttaggacccatgggggacccatggggtgtcgtgtggtggtctggtttgatccgcgatatttctccgttcgtagtgtagatctgatcgaatccctggtgaaatcgttgatcgtgctattcgtgtgagggttcttaggtttggagttgtggaggtagttctgatcggtttgtaggtgagattttccccatgattttgcttggctcgtttgtcttggttagattagatctgcccgcattttgttcgatatttctgatgcagatatgatgaataatttcgtccttgtatcccgcgtccgtatgtgtattaagtttgcaggtgctagttaggtttttcctactgatttgtcttatccattctgtttagcttgcaaggtttggtaatggtccggcatgtttgtctctatagattagagtagaataagattatctcaacaagctgttggcttatcaattttggatctgcatgtgtttcgcatctatatctttgcaattaagatggtagatggacatatgctcctgttgagttgatgttgtaccttttacctgaggtctgaggaacatgcatcctcctgctactttgtgcttatacagatcatcaagattatgcagctaatattcgatcagtttctagtatctacatggtaaacttgcatgcacttgctacttatttttgatatacttggatgataacatatgctgctggttgattcctacctacatgatgaacattttacaggccattagtgtctgtctgtatgtgttgttcctgtttgcttcagtctatttctgtttcattcctagtttattggttctctgctagatacttaccctgctgggcttagttatcatcttatctcgaatgcattttcatgtttatagatgaatatacactcagataggtgtagatgtatgctactgtttctctacgttgctgtaggttttacctgtggcaactgcatactcctgttgcttcgctagatatgtatgtgcttatatagattaagatatgtgtgatggttctttagtatatctgatgatcatgtatgctcttttaacttcttgctacacttggtaacatgctgtgatgctgtttgttgattctgtagcactaccaatgatgaccttatctctctttgtatatgatgtttctgtttgtttgaggcttgtgttactgctagttacttaccctgttgcctggctaatcttctgcagatgcagatc

SEQ ID NO: 30 - DNA, 1853 bp, Oryza sativa
  misc_feature 1..1853: note = Exemplary Oryza sativa Cytochrome c gene promoter (OsCc1)
  source 1..1853: mol_type = unassigned DNA
gaattcggatcttcgaaggtaggctgcagttcttgaattgttgaattattattatcttcatcttcattcatctgtaactactgattcatctggtttgttattaccgatcgtaatgccgttgttttgtcaaaaaaaaaaaaggagatcggtttgttattaccgatcataatgctgttcttttataaaaaaaaaacatggatctattggcataatctttttgcgccaggtactccgaccattactcggttaccgacgaaagccggtgagatttggataaacttcgccaaaaatttaaatttccgtttgatctctcaaacgtgggctggtttaggcctgtttaatgtttagacacatgtatggagtactaaatattaataaaaaaaataattacacagatcgtgtgtaaattgcgagataaatcttttaagcctaattgctccatgaacaatgtggtgttacagtaaacatttgctaatgacagattaattaggcttaataaattcgtctcacagtttacaggtgaaatatgtaatttatttattattaagtctatatataatactttaaatacgtgaccgtatatcccgatgggagacacgtaaaactttttaaccaagttctaaacacaaccttgcttcacagtttcttgatctctatgggtaggggtgggcagaaaaagaccgaaccgaaagaccgaaccgaaaaggccgagaccgagaccgaaaagatcgagaccgagaaattcggtcctaggtaatgaaagaccgaattttgttcggtcaatttggttagttttctcgggtaaccgaatagaccgaaaagaccaaattatcagaaaatatctaaatacaatctacaacccactatgtttaataggattaaactctaattttttacatccctacttcttttaggcatgcaacctaataagagtctttactcataagtgcttacgaaatttttttgtgatttttgtgttgaaaatttccattatttctttgcatatatgaaaatgttgttgaatttcggtcaggaccgagaccgagactgaatttgtcagtcctaacattttttcaccgaaattcagtcttcacttttcaaagactgaaaagaccgaaagactgaagaccgagaccgaaattttcggttagaccgaatgcccacccctatctacgggcttgataagatcaataaccgtaattaccgaagcggttgcgtgacttgctgttgcatttgtcaaccctaacatagtactacctccgtttcaaggttccgtttcagagtttgtaaaactttcctagtattaacccatgttttaacttgcaacgggaggaagttaacatcctatacgcctgaaatccctttaaaaaaaaagaacatttatacgctggaaccgattctgaaccggtccgtccacccaccgacccaccaacggtgcgatttccaccgtccaccaaacgcgagccgcctccaccctccacctatcgagtcaaagacgacgactctaccagagcacgtggacccggtccacgaacggaacgcccttacaccgaatgggccgttgggtgtccacgcctcccacacccacaccccccttgcctttttctgcaagacacggaaaccttctggaaccgcgtggattccccgaaacgcccctgcccccacgctccacccgttcaataattctaggggtattatcgtagtttcgccacctgcccttccgccgcgctggtgtatactagggcacgcgctcctcggaatcgccacgagcccacgagccagaaaaaaaaggaaaaaaagagagtcgtagttcgcctcttcttcctcctctcgttctcgcggcggcggcggag

SEQ ID NO: 31 - DNA, 921 bp, unidentified
  misc_feature 1..921: note = Exemplary Epipremnum Aureum Ubiquitin promoter (rrEaUbi1)
  source 1..921: mol_type = unassigned DNA
acagagtaatccttcaagacacataataactcacgaatgtaaagaactacaaacacacaaaattgttcaaaaaaatttatgcaagaaattttttaagttacattatagcacattcacataagtgagtgtcaaattgatggataatctcctatattttataaaaaattacactcacatgagtacatgttataatctaataagaaatcattatagtatataaattatttctcatgtttatgatagcacgcaccacttgcaacacgtaaagtatgtacgtgactacatgtacaaatctaaataatgttggggtaagataaaaatttaacaaatttaacatgtaaatacttttgggtcagacttaatgcatcgtttaagaaaagcgatgctggatcgcacacccatgatcaaataatttcttgtaaatatctttttgaaaaattttaagttaattaaatatactcccgttaaaatatttttttataaaaaatctgctacataaatgtcatttatatccccattgcatatgtatatatacatatatataccatatatgctggttatatataaagagatatatttttaacaaagtaattatttttaactgacagttattggtctggggcaaatttaatttaacagggtatatatgcaatttacccaaaactttttaatcttttcccgtggggcgaaggagcagaccggctccgatccaaacattcgccctcgtattccgtctcctcaatctctctctctctctctctctttcttcgctccctcctgcaagcaaaagccaatatttttcttcctccaaatccccctttcctctacaaacaacacccctcactgcttctcttgcttctctccccgcctcagaatcaccagatcgcaactcgatctagggtttagaaccggtacgtctcc

SEQ ID NO: 32 - DNA, 930 bp, unidentified
  misc_feature 1..930: note = Exemplary Epipremnum Aureum Ubiquitin promoter (rrEaUbi3)
  source 1..930: mol_type = unassigned DNA
ggggtgcgacaacattacctagttcattagtgggaccatctgcagattgaggactcttggatcatccgaaagtagttccagtgccttgactcagacttattagagtaacactagagcggcaccgaccatttctcgacgggatcgagttctttccagttaggaggagttggtggagacactaaaaatagggttcgttttgaccctgggtgggtctgcaacagacgagaatgtgcgaaaatgacaatgacatcactttaatttggagacgagtagtgggcccagtaagaattttgtggtgccatcattattaagcatgttaaggttgggagtcttttgataccttattgggcttatttgggcttagttttattttttttttcttcatattttttatatgattttcatgcatttttttatgtgtgaggaatattttggtcataaaatgtcttttacagttagagttatgagagagtttataaatatgttctataactctcttttttaattattggaaaatcttgttgcgaattttgagtattttattgtactctatgagagaggttgagaggaccgctacttacggtcatccgcgagagacggggacttacattcctcatcgcccacccctttgctgcctttgtgactgtgttcctcgttaagaagtctgatccctgaaaagttgctaaagatacctctatcacatctgacgtgttgtgaggatcgtaatggtgtaatcacaactcaaatcagatgtcggacgggcttgatttcatactggtagattcttttggaacccgtgattgcacaacgtatggctgggggggtacgtgtcgtcgtggcactatgtaaggcaagctgaagtgagcataaacaacaagtagacctcgatggatgagtttgtcatcttcaggcattcatcaatgtggacgc

SEQ ID NO: 33 - DNA, 352 bp, unidentified
  misc_feature 1..352: note = Exemplary Epipremnum Aureum Ubiquitin promoter (rrEaUbi4)
  source 1..352: mol_type = unassigned DNA
gcaagttgcgtaatcgtgctccgttgctgagtggtttgttttggactcctggttctggctcgtcagacaactggtaaacatagaaataatcaactaagctgcaaatttcccgcaagggaagttggcggcagacaattgaactgtaacatttgaatgtaatggtttttcggttgttgacaggataattttagttaacaccccggctctctcacccggagttcctgcctgtgccttgcgggcattgggcttttgaactgtgtttggactcatggaattgcatgaaaacttggagcgtgaggttgcacgttagaagtgtatagaagtgccttaggagttagctccgggtgtggga

SEQ ID NO: 34 - DNA, 986 bp, unidentified
  misc_feature 1..986: note = Exemplary Epipremnum Aureum Actin promoter (rrEaAct1)
  source 1..986: mol_type = unassigned DNA
tctgttgtgacatgtgacgtgaatctaaagaaacactcgctatttgcattatttttcttgtattttcagtgaagcaaagtgtcaaagttgcctatcgttggtcaagatcctggatctgttggggatctctccttacattgcaatttcctcttgtccttattgttttaatttcggaaagcgctatttgttgcttgctttgttgcagtttacatcatcccttcttgatgctctttggggggaaatctctctgggacattcgataatatttggaaaaaaatagtctgcgagccagaagccccagtgcgctctcgtttgtttttcgtctcatgcttcttaatcttgtatttggcatttgggaagagtgacacaggatatgctatctaattagtaaatgaatgtgtttatcgtgcggacaactaattattcagatggatgaaattcttgaagatttatgttaagaataaatcattatgcaataatttcctaaatgtcaattgatattgcatcggatttcacatgcaccagtaaaactagtacttacctgtggttcatgacaaacacgattttttttaatttttctaatgcaatttactttttctgctcatactttctcttaaagtaacatccatctccacttgtttttttttcctttctcaaatatatcttgatccacacttaccgacaagcctgtactggtttatctgattgttaaatttgatgttacatttgaatgggaagagatatcatgttagttcggttctagcattaaaatgcctagtacatcttactccttttgcagaatgactttctttatacatatggtacgttatttttcttgaaatggagcttgcccaagcagaatttcttttttcatggatgatggttgtcgttggtagtttaattttatcattaacctttcacgtcttacatatttctcagatattggtgaatattttaatctgaaacgtaaagtgagcaggtgtaga

SEQ ID NO: 35 - DNA, 989 bp, unidentified
  misc_feature 1..989: note = Exemplary Epipremnum Aureum Actin promoter (rrEaAct2)
  source 1..989: mol_type = unassigned DNA
acaccatcaccctcattggtttctgtagcatgactctgagctacgatggaagatccaagttccaaaataaaaatagtccctggtgtcactattgggtcgctcaagcaaggcatatattgtctaagttgacctgaaaattgcatgaccaaatctgattcccgctcacggccctgtccgcgacgtcactcgtgaaactccctattagagggagagtggagcatcatgcttggaagctaaaaaaaaatggatgatgtcaaaattccaaactaacaataagtaatgagctgtattgggcaaataatactaatatagaagtagtaagtaaaagagagagaaaaaagagtcaataaaaaaaatgcaacaaaaggttttgtgcttaccgaccgctgtccgtggcacttcccggttcgtgggggacatttgttggcaaatatcttttttattattattcaaaaaaaatgaaaaggaagggagataagaaaagacaagagactgctctcccacaccttaatgcaactcaggttggttcacttatggtgcaacacaaggtaacctgcaatcaaaaggtctgggcagctggattttgtgctgtcttactttagaagcacaactctttgacatatgctttggtggaatttatcaaaggaaaagctcctgatgttgtaaacagtgggtcaataacacaacaggctaaaacagatttcatgaaaaattcattctctggtctgctatagaaaagttcttcacagtgattttggggctaccagatgttcagaggtggtattcagctagcggcaatttcaagctgggttgcagtttgaaggcagaaaagagacaggctgttctttgcctgatcagggattgtcccccatctctctccctctgtcttttctctccctcctgcactcccatcagaaaatagcagggagagagagactgatgggtctttccctctctcactgatttttccctttctcctggttttctct

SEQ ID NO: 36 - DNA, 923 bp, unidentified
  misc_feature 1..923: note = Exemplary Epipremnum Aureum Histone H3 promoter (rrEaH32)
  source 1..923: mol_type = unassigned DNA
atggctgcattacctgacgtacaatattattggtaggtaattcgagattaactatgaaatatgtatatgtgtctcacaactaagtaatggccaacttagttaaccaggttatgaacaagttaaagttggtgtcaaactctggattaacttcagagtaaccactctctacttagaacccaaaacttatgtaagttaatactaatgagtaatctctggactaacccaccacaccaattcatgacttttggaagaaagattacttattaatccgaataatttggaccccctttttgaaaataattattgagttaattctgaactattaaatatttcatattattaataatcattttaaataaaagctgctgatcttagttgtaattttttttactattaacaaagagagagataaacgcatttttttctatttttataccaaaattaacccatattcaaattttggggatgacacatgaattaagctagtttctcattagaaaaagatcttagccttacttattaggggtacatagataatttaatttttttaaatgttttcacgtaatttcaaaccatttaggccaaagcgggccgaattcaaattcgtgggctcggtgtcacgttggtccagccagagcagtgttatcagcttcctacctggtgaaggtacgccattggctgttgtccgacgacgcggatcaagttgcataaacaaattcgcaccgtccgatgaaagcgaatgatcccgattcactcaaggggcccccgctgcggcagcggcggagaaaatttcgaactctccgccaaaagggctcctctctctctctctctctacaaatactcgccaaaggctccccctttgttctacccaagcagtcctcgctgctccagatcgagaggcatccagagagcgtccgaaagaa

SEQ ID NO: 37 - DNA, 903 bp, unidentified
  misc_feature 1..903: note = Exemplary Epipremnum Aureum Histone H3 promoter (rrEaH31)
  source 1..903: mol_type = unassigned DNA
tgttacaaaacagaagaaatttgacatatgtgttgaacataatcttgtcctaatatttttttattttttttaaaattttaaagtacttaaaaatattatctcttaaaatcaacgtccatcacacaatttgtaaatttggaccaagtcaacctgagttgattgacttagttcatattcaattatttagtatatacgattcaatacaaattatttaaataataatataatatttaaaatataatttacatattttataaaaattaaaaataataaaaatttaaatatgtgacttaataagtcacaagagttttgatatgtggataaaagtttctatagacaaacaagatttttttgaataaaaattatctactaaattgtaaaagttttatgagattttaagatttgttatttataaacataaaatttttaatgttaaataaaataaaataattgatgaaaatttaaattatcctattatattgtcaaaaaattcacaagagaagagtggcagtcaaaagttatcctcgaattattttcttaatatagataaaaaaaagatctcgagagaatttaaaatttagaaacccctggcccaccctagcccagaaagctcgccagccgcgctggccgggcccgcacttacgctcccaagagggagcttggccaaggtcgaaagtgacggcgatcgcgatccgcgtgctattcctcaggatcatctcaaccgttctttgagacaaatcgacgatctcgactaaccaccgagaaattcaaaagttccaaaaccggctcccgcctttcgtgcgcctacaagtatccatcccttccctcagggcttgaatcgtctccacccctccgaacacaaagcatttcctcctgctgcaccgaaaccctaggccctcgttc

SEQ ID NO: 38 - DNA, 750 bp, Cauliflower mosaic virus
  misc_feature 1..750: note = Exemplary Cauliflower Mosaic virus promoter (2x CaMV35S)
  source 1..750: mol_type = unassigned DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggaca

SEQ ID NO: 39 - DNA, 180 bp, unidentified
  misc_feature 1..180: note = Exemplary Agrobacterium tumefaciens Nopaline synthase gene promoter (NOS)
  source 1..180: mol_type = unassigned DNA
gaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaatta

SEQ ID NO: 40 - DNA, 359 bp, unidentified
  misc_feature 1..359: note = Exemplary Agrobacterium tumefaciens Octopine synthase gene promoter (Ocs)
  source 1..359: mol_type = unassigned DNA
ctgaaagcgacgttggatgttaacatctacaaattgccttttcttatcgaccatgtacgtaagcgcttacgtttttggtggacccttgaggaaactggtagctgttgtgggcctgtgctctcaagatggatcattaatttccaccttcacctacgatggggggcatcgcaccggtgagtaatattgtacggctaagagcgaatttggcctgtaagatcctttttaccgacaactcatccacattgatggtaggcagaaagttaaaggattatcgcaagtcaatacttgcccattcattgatctatttaaaggtgtggcctcaaggataatcgccaaaccattatatttgcaatctacca

SEQ ID NO: 41 - DNA, 383 bp, unidentified
  misc_feature 1..383: note = Exemplary Agrobacterium tumefaciens Mannopine synthase gene promoter (Mas)
  source 1..383: mol_type = unassigned DNA
atttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcg

SEQ ID NO: 42 - DNA, 516 bp, Cassava vein mosaic virus
  misc_feature 1..516: note = Exemplary Cassava Vein Mosaic Virus promoter (CsCMV)
  source 1..516: mol_type = unassigned DNA
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgt

SEQ ID NO: 43 - DNA, 863 bp, Arabidopsis thaliana
  misc_feature 1..863: note = Exemplary Arabidopsis thaliana Actin 2 promoter (AthAct2)
  source 1..863: mol_type = unassigned DNA
aggagtcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaatactgtatttttacaacaattaccaacaacaacaaacaacaaacaacattacaattactatttacaattac

SEQ ID NO: 44 - DNA, 822 bp, Solanum lycopersicum
  misc_feature 1..822: note = Exemplary Solanum lycopersicum Histone H4 promoter (SlHis4)
  source 1..822: mol_type = unassigned DNA
aggagaatatcatttttaagtaaaattttgaattcaaatgttacgtgtattatttaattcatcaatttgccttgtcatagcgagtacattacaaacatcacatatatttgattgattgtcaaaaaatatcaaaatatatatcaattttaagaggtataggtgtctaatatgtactagccctaatttaaatatctaaattaattattcggatgaatctatataccatctttttaatggacacccaaaatcacacatcaaacatcatatacatgttgaaaacatattattgatatagctacatatatgttttaatataaataaaagacgagtcatatattcaaaaattaagaatcaaataattttaatttatttaatattcaaaacttaatactatttaaatttagatattctaattttaatacacgtctgataaaatagatgaggactaaataaataatttgagactatcttttctttatttggcggcccacaaataatttagattctcgtaaccccctctttttctctcactgaaaaagcacaatccgtgtccaaacacaaagaagcactcgacaccgtagatctccattcagatcaacggcttatattcagttttctccattcacgtggatcgacattcttatccgtccgattatcaataaatttcccaaaatttagcggccatgattttaaccccgcctcatttcaaaccgcccacgaaatcctcgacgcccaaattcaccaactataaatagccaccaccatccccttcatcaatcatcaaatttcataaccctagaatcatcacctttttcaaatttc

SEQ ID NO: 45 - DNA, 692 bp, Arabidopsis thaliana
  misc_feature 1..692: note = Exemplary Arabidopsis thaliana Light-harvesting chlorophyll-protein complex II subunit B1 Promoter (AthLHB1B1)
  source 1..692: mol_type = unassigned DNA
aggagatatgactggtaagtttttcttgccaatacgaattagaaaacatgtctttgaagatgaactgtattttttttttttactttgttgtcattttaatgtactttcttatcaggattaaatcttctgtaatttagagtagtttttttaacaagataattaacaaacttagagtaatgaaaattgagatgttcagttttcactcatatttcacattttggtgaaagagtgggtagtatgcaacgttctaagtatgtttggactttgtatcatgttgttttgattctttgacgacatgtctatttgggaaacaccaatgacgtgtaccttgagactgatacgattcaaagggatagaaacacgtcagatttacaagtggcacctcttcaatggacaatgggtattccaatatgctaagatgctacgagatatctaatttatctaacacaactcaattccaaaccaaaaatctgatgccagctcgacaagacaaaaaatctaagctcaaaaatgtcaacaaccaatagaaatcaaggcattgacgatatcacgagataagcaaattaaatcttcaagttttgcaattcatatgtacgttataaatacccaaaaacctcaccgtaacctagctatccaatttcatcacatcttattaactaaagagccttttacttgcgccacactctcaccgc

SEQ ID NO: 46 - DNA, 1303 bp, unidentified
  misc_feature 1..1303: note = Exemplary Epipremnum aureum ribulose bisphosphate carboxylase/oxygenase activase 2 promoter (rrEaCons1)
  source 1..1303: mol_type = unassigned DNA
acctcaaccttcgctcacagtgaaggcttgaaactcgctttttaacattgtaagtgggctgattttgaactcatctcatcgtaaatctttaagctttgacttcccacgatgttgtccagtctattagattttttatggtttttttttcttttttcgctgaaagttcctacttaaaatagtcacccactaggtacagaagagtcagctacatgaaaaataccttaatatagaaaaacgtatttattgtattaaaatttgaaccctccccacttaaaatgatgcgtaccacttagacctagttgagatttattgttgcacctgggagagagttgaatagggtccggattcccacttagtttctctggaatctagatagggcggtcagctttatcttaattagtgacaaggcactagttggagttagtttttatattgaacatactcttaaacttttagttccctattttgagagaaagtatttgaagtaattttaaacttttggttaaatcttccacttttgaccaaaagttcaaaattaaagtttcccaagttcaagaaagaatggtatcattagcccatataagaactaaattaaaatcagtttgattcattcttattaagctccaacatactcaacagcacaaccaacagcatgacttgtgtaaactgaaaaactcagagagagagagatagagactctgaacgagtggtgctgagcagcagtggctgcttcatgaagagtttggcgtgacgacaaaaccatcaaaaacacagaagaggaatttcattgccgacaatcaccatgtctctgtaatactgctggtcctgatgaaatgcttgaaggaaaaaaaactggcattaaagaggaggggaaaaaaccgaaaattttagtggagtcgggaagcccgggaacccgaaccattcctggcgtctgacgtcctccgctgccgagaggatgctgtagctgatgggccccacttccccacactccccaacttccaacgtcaggacacgactctatctgcgcagaagcaaccaaccctgatgcgccacgtgtcgccccaccccaatccgcagtgtgtggccgttgtggccctcgcgatccaatccacaggatgcttcactctcctcctctcctccgcaagccaaacgggaaaataacggagcagggcagactccagagcctccgcaggccgctttatatataactcgccctcccacgcctcctacggtcatcactgccgcgaggagctttgcttttggtggacgcggcgatctccccccatctccttctcggtcttcc

SEQ ID NO: 47 - DNA, 1167 bp, unidentified
  misc_feature 1..1167: note = Exemplary Epipremnum aureum Metallothionein-like protein type 3 promoter (rrEaCons2)
  source 1..1167: mol_type = unassigned DNA
aggaacaagtgccacctgagccaaggcgctcattggcgtcttgatagtttcttttatggtatacatgctgttgtaagaatcttaatgttttaaatttgcatctgcatgtatatatccacgttttggtgtaatatccacgtctatacccttgtgaaaggtatctgtatgcatccaagtatagttaaatcactttttaaaatttacagctatgtcccttgtaaagctataatgacatttttgtgcatctagaaagagtactcactcggggactcttctaacagacaagcacatgatgagaaatttgcacccgcacaattcaaatttgattctgaaagacttgcaacttacaaactatcttaagtacgtacgaccacaaattatctcaagtgtactctttgttccacaaataacttttacattgacactatttaaggacgacactgatcagagataaaatgacaaaatgaaaggggactcatctaagttagacaaatcccgaaacttatttcatataccctaagaacacttgcccccctaattaacgacggtacatgagtaacatgtttgcttttcacatgaatacaaatggcagtacatatatgtaagctagcaagaaggatatgtgggtgataattatctgtatatggtccgtatccacctccctctctagtatctccatcacgtagccagaggtcatcggatttgtacaccagttgcatgtgcctgtgcatctgttgccagttgcgtgtgacagtgcagctgtgtattgccacaaaaaaaaaaggaataaaaaggtagtgcaactgggtaacggtgcaaggatagccgtgtctgcccatctgaacccaaaagggcgacgacgacgactcggggaggtgaaagaagaggaactggcgtgagagctggtggggcagcccccctcctctccaccataattgagattcctttggaagcttcccccatggaggcgtgtgcccgtcacacacaggaggcagaagcccttcccctccatctctccttgtgccgtgtgcggctgcccatccaacccctggggcctataaatatcgtcgcaggggcagaagcccctccagcatagctgaagcttgagtagttcagagatatagctctctttgatctccagagaggctccctcctgacatcaccacc

SEQ ID NO: 48 - DNA, 929 bp, unidentified
  misc_feature 1..929: note = Exemplary Epipremnum aureum abscisic stress-ripening protein 2-like promoter (rrEaCons3)
  source 1..929: mol_type = unassigned DNA
gttccactcgaggcaggaaaaatctctggatttggacacttaaccgacccccattaacaccccacctcacatcagagcacggtttgcccactcaacttgtcaggcaaaccacatcttatctcaaaagctatgagttacaacgtcagataactaatttaaataataatataaatttaaaatataaattatattttttattaaattaaaagaataatattttttaaatatctaattttatccaatcaaattcaagttcaactgatctatattaaataaaaaaattaatacgaatccaaattttaagttgacaaataaatgaattttgaataaaagaatcacaaataaaaaattacgttttcttggcgtatatcaccatgcttgtcttcgtttaagagatttaagcaatcatggacgtctgcttatccacggatgtgaaatattaaatgataaaatactatattatcttatattatagaaaaataaattttaaatgagaagtgggtatttattatgttttcattcaacatacgtgcgaaagttttatctagatagattagcgttagcatcactcaagaattttttttattttcttaactgcttcaaaaaaagaaatataaagggattggcccacgttaattagctagaaaaagtgggattgaaacgggtgttatccacttcacattctgtgagcgaatccgatgcgtgaagccccgccatcctgacccgaccgctgttcccccctacccacgaagaagccgtctgtccgtctcttcaatctctatacttccccttcgcctgctgcgtacactcccgtggctataaataaccaccacagcctctctgatttcttcgtacccattactgcaacacctctacagctactagccgtgtcgcccgcccccccttaaggtcattctaccactgccagt

SEQ ID NO: 49 - DNA, 841 bp, unidentified
  misc_feature 1..841: note = Exemplary Epipremnum aureum RNA-binding protein cabeza-like promoter (rrEaCons4)
  source 1..841: mol_type = unassigned DNA
gcaacaatgacgcggattcagcccgccaaacagataccattaactcggttcacttgtttaagaaagcgttgtagatttttttttaaaatttattaataaaattttaccgcccccaaagcccaaactaatgttatcaagttggaatctgaaaaaaaaatagattcgagagaaagatattaattcaatcaaaatacaaataattcatgaaaggttctgaatgtatcgtcgatctttaatataattaaatattaattgtaaatcatataaaaactattaattgactagttccaatagccagtccttgtcactcttggctgcattgccgggtatcggatattggcaccgcggagaacgcgagaggtgcctcaccgccaacatggaaggcgcttgcgcctttcggttgactcccgaggtaaacaaggggccaggggcatccacgtaaacacgccctcccccgggcccaggggtatccacgtaaacacgcccttcagatatgtctgtgtcgcttgcgcggtccccgccccgctcgttcccttccctgtgataagcacaaagccacgaaccctgttctgggcctaaacgggccaccaaacgatcgggggatccaatccagcacgagttccactgttccctcaccccatctaaatcttaatttgctccagctccacgagggtaccattacacagctcccgaaaacgtccaccagttcgcacaggctcgtcgaggggaacacgatagtgtctagtgcggggtccatgggcccatccagtactgccggccagtccacgaagcccaacggggaccctggttgaacccaagcgtggggttacaaacgctcgag

SEQ ID NO: 50 - DNA, 906 bp, unidentified
  misc_feature 1..906: note = Exemplary Epipremnum aureum leaf patterning promoter (rrEaAs21)
  source 1..906: mol_type = unassigned DNA
gctccgtcccttttcccttttctttccatttctaccatgcgtgtcagcgtgtgcgtccattgctcgaactgtgtctgcacgtgttcatgtgatcatcagaagtcttgttcgcaggcccaccgttttcgatttggagatccccggacataatccggaagagatcttcttttttagcacatgaacatacagtaatgcgagaatggaaggagtgagaaaatatcctttgaatcccggttgcatcccgaatcctaccgagaaagagaggatctctatctcaagcagtgtaagaagagctcacggtggtctttcccgatcatgtccggaggcatgtgatctcaagtgctgtggtgcaagtaatccccttagaaggttatgatctccgttccgtatccatcaccgtctttcgtacttcatgggtttctcttcccttctctctcctatccgtgtatcttctcagatttgtatgggagatactgtatggggaggagtagagtctgggttgtattcagttccctccattgcccttttagacaagagaaaggaaaaacagtgaattccatgtgttcttctgtccaaccgtgtcgccttgctgcgaatagtcctagcaattgcactgttgccatgccttcctgtcactgtaagatgacactctactctgtgtgtcttttttggtattatctctaagggcaatccgcacacgttcccgttcatttacttcatgtggaaaagaaaaaagtttgtttctttctgaaaaaaatcatggaagataattgttttgcccactcatttgctactatatattctaccttaatttgtttgcaacgggtcaggttgtttaaatctgactgtttaaaggctctatcttttggacaggaattgatcatatataagcagccgtgtgtggtt

SEQ ID NO: 51 - DNA, 980 bp, unidentified
  misc_feature 1..980: note = Exemplary Epipremnum aureum leaf age dependent promoter (rrEaKan22)
  source 1..980: mol_type = unassigned DNA
ccatcgctattcttgtattgtcacgaatgccacccctagataatttatttgtgaaaatatctttgaaatacaatttttgtgcataaattctcaaaagatggcattcatatgagaataagggtgacaaatgcgtaatgtaacaatgacatatttgtaaaaaaaattcatatctaattttccaacattaatctatctaaaatattataatatcatatctaatagatgttgaccatacgtgaggcatttggcactaggcctacccaaggaggatgcaaatgtgtttttaatggagttactttgcacatcttttatacaaggggggcatcgttacaaaaactcaaaattaacttgtgagaggccggctttatctttttatggcccgtaaagcggaaatatgagaagtggagaaatggaataggagacaggaaggaagggatgcacacaaagctaaaatgttagatcagaacttcactttttatcaaaaagaaaatcagtgggaaaaagaataaaaaaaaagaatcgaagccttctctctctctctctctctctctctctctctctctctctctctctccttctatgtgtgtttgtccacaccccacgtccacaaaagaaacatactcctactttctcctctatttctctctcctggcagccaagaccattcataccgagtgtcattttcctgcacatacttccccttcatacaagaagtaaccacttccactctccccgtttcaagacatttacctcccctccaatccctcgttccccaactcccctcccaaaaccttcctgttcatctagaacacccatctgctccacacctcctacccttcccacactcccaacgggaagaagaactcagtgtacgagaagaaacccagagtcccgtctgcggcggcgcaggcggagggtagggagggagggagagagagagtgagtgtgtgtgtgtgtgtgtgagagagagagagagaggt

SEQ ID NO: 52 - DNA, 849 bp, unidentified
  misc_feature 1..849: note = Exemplary Epipremnum aureum leaf age dependent promoter (rrEaDPA41)
  source 1..849: mol_type = unassigned DNA
tgctccaattacatttgccatctgaaaatatatgccacagtctggttaatttttaaagaaaaaaaataatattccagcagaagaatggatcgctggatcaagttttttttctgcccaattaaaagttgaaatggtggtccaaaatgatttcttattcggaaaattgaatattttaaaataatatatatcgtactgacacgtgagaatagcgaaaaggacgagctcacatgagcctaaccagatggtgcatggtcccggtccagctctcccttcccgtctttgcacggctccaattcctctcccagctttatctcttccatctcggttccctttcatctcttctccccagctgtaatacgagaggaataccagtgcaggtactcgcgcttcggcgtctctgtccgccgctcctcctcctcactccttcaccagatctgttataagctgaagcctctcaaaccctaatctcgaatgtccccaggggtatgagcccatctgcagcctttccatcccagagatcgatgggaagccatctaatcctgtagttctgcctgctatagcactgagcagcgggagagcaggccatgcaccgatccaccccttcggctgtatcctcctcctcttctgatctcctcttctcccccctccctctcgttgtgcaagcagttcagtgggatgcccgcatctctctctctttcccccatattctcccctccgcccccgctttccgtttctttctcatcttacaggtgtagagagagagagagagagagagagagagagagagctgtgagttaacacagtaaaagaaggcgtaggatttgcacagtcgtcgtctgtcgtctgaga

SEQ ID NO: 53 - DNA, 967 bp, unidentified
  misc_feature 1..967: note = Exemplary Epipremnum aureum salicyclic acid stress responsive promoter (rrEaPR11)
  source 1..967: mol_type = unassigned DNA
ggaattcccacagaatcagattcgggtacaaatgcgccaggaggaatacacgccgcccaaggttcccaaactacattattaatacaagccttaattagatcaagtgatcccgtcagtgataaaaataataaacaaataatatgttaggtttttttatttttttatttttataaaaagaatattgcattaaacctgtagttaatttatttatatataagctttaatgcaacagagagatttgttgctaaaattttgtaaggagcttagattattatgcccctcttttttcatagggtgagaggggtcctccttgtagtaggtttctagaattctaaatagtcacttaatcaagtaaattatagttcaaataagtgaaatggatgtttaattaggcaaaaatcagatctgtaggacagaaatttcttaattagggacataattaattacgatcttggctttcatagaacattataatataaatatttaactgggaaccaaaaaaatctacaaaggtgtactttacacagacaaatttcacaatgttttttcagaatatataagatttttcttagagatatagtaaagctcacttaataaaagagatcacgagataagatctagttgatgataataattattataatactttatttaacaaaaattaaaataattttaattattatgataattataaaaatatttataataacatctttcataaattaactctaagttaatttacacggttgtggttatgattatttaaaaattaaacaaagattaacaaatttataattataattaatgaagttgtaaaatttaattagaataatctcaactacagtatcaaacagtcgacgttgttggtggacgttcccagtagagagaaagagagggagagagagagagggaggtgggcgggggaagagagagaaagcggaacccggacaaacaactacaaagctcc

SEQ ID NO: 54 - DNA, 951 bp, Arabidopsis thaliana
  misc_feature 1..951: note = Exemplary Arabidopsis thaliana quick response stress responsive promoter (rrAtZat12)
  source 1..951: mol_type = unassigned DNA
aaggtataacgaagatttgttccgcgtggaaaaggcattaaaagtgccacgtcactctctctttttattttatgattttcgtatctcttcttctacttgcttcccacgtttccatcaagtttccgtacatatcttcttgttatctgatccacgcgatctttcaacgcgtacttttcacgtatttgtgttgtcatgcctttgctgggattgtgttagatgctcattgctgacggtagtttttagagaacattctagaaagaaactatttttctaacaaaaccacgaactttgttttctagttattccactttctagaatacacctgaccaaattagaattctagaaatgaattttaaataaaccaaaacacctaaacgaaaagcaaaccataggtttttggttttaacatatttcaaattcataaaagtgaaaccaacctacaccatattaaccaatatttattagagtttttatatgttttatgatattgttcaaaacttcaaaagagatttattcatataacatacctataccataccaatgaatattaaaattatgaattagtatccttatattatatgaagtcaatcaaaaaacttagaagcatttcaaacggaatcaaaccattcatatatgaagtattattattatatctagaaggtgttgattttaaactattccgtataatatatctagaagacggctccgcgcgtggggaatgcatcaaactcagagagtttaatagctttttttggttgacgtcaactactcaaaagagtttagtttttgatgtgtatatatccaaataaaatatctttaaaaagaaaataataataataaatggtttcgagaaaacacgaggaagattctcatccaaccgaaacgactctttcgtttttagtagtctcttaagctacgcggtgtcgcaaatcgtgaccacataacccgttt

SEQ ID NO: 55 - DNA, 942 bp, unidentified
  misc_feature 1..942: note = Exemplary Epipremnum aureum auxin signaling responsive promoter (rrEaPin12)
  source 1..942: mol_type = unassigned DNA
gctacttctttcagccacgcactgcgcttcaaaacttccacggtaccatagtcgagtttgacgagaaaatgtcgaacttgtggagaggaagagaaagtgatcccatgagaattcagaataaatccaagtagcagatgaacagtactcgtattgatgcgctacgtaacgtataatacctggcgaaaaccataaaacccaagagagcgaatcttaagaagtactgttgtttttttttctggggacacggtgagaagagaagcctagcgttctcccccaaacagagttctctctcctccctcccctcctgtctaagttctaaaaaggtggcgtggtcgggcacattgcttcgtctcttgcttcccgttcctgaacccatttaaagcaggtgttgctttgttgtctgcctacagagctccacaaaatagtaagcagatacacaacaacacgtacgccatcgccataactctccttcgcctctcccagttgctggttacatctgttctactacgagcacctgtcccccattttctttccctcctctctgctttttccctgtttcgcgctctgtcaccgcttctcccttctctttccccctctgcactgatggttaacgtgcttaaaatcacttcagttgtcctcttctaataagcagggttcttcattgagaagaatctccacaggtaagcaaacatcacctcgttaggcttctcattccacttcttcacaaagggtccaccgcaaacccagatagcaagccctgcttcgtcgtttgcccctgttccatttccatttccacccggggtcactctcagtcatggtttcccgggggaagcagtgagctgctttgttcttactgaagccaggcacacagggccttccaccaccgccaccgttctccctcgttccctgcatcagaagagccacgtggtgttcttgcaggat

SEQ ID NO: 56 - DNA, 755 bp, Rice tungro bacilliform virus
  misc_feature 1..755: note = Exemplary Rice tungro bacilliform virus promoter (RTBV)
  source 1..755: mol_type = unassigned DNA
agtagtaatatttaatgagcttgaaggaggatatcaactctctccaaggtttattggacacctttatgctcatggttttattaaacaaataaacttcacaaccaaggttcctgaagggctaccgccaatcatagcggaaaaacttcaagactataagttccctggatcaaataccgtcttaatagaacgagagattcctcgctggaacttcaatgaaatgaaaagagaaacacagatgaggaccaacttatatatcttcaagaattatcgctgtttctatggctattcaccattaaggccatacgaacctataactcctgaagaatttgggtttgattactacagttgggaaaatatggttgatgaagatgaaggagaagttgtatacatctccaagtatactaagattatcaaagtcactaaagagcatgcatgggcttggccagaacatgatggagacacaatgtcctgcaccacatcaatagaagatgaatggatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtataatgaccagtgtgcccctggactccagtatataaggagcaccagagtagtgtaatagatcatcgatcaagcaagcgagagctcaaacttctaagagagcaa

SEQ ID NO: 57 - DNA, 870 bp, unidentified
  misc_feature 1..870: note = Exemplary Agrobacterium rhizogenes promoter (RolC)
  source 1..870: mol_type = unassigned DNA
aaagttggcccgctattggatttcgcgaaagcggcattggcaaacgtgaagattgctgcattcaagatactttttctattttctggttaagatgtaaagtattgccacaatcatattaattactaacattgtatatgtaatatagtgcggagattatctatgccaaaatgatgtattaataatagcaataataatatgtgttaatctttttcaatcgggaatacgtttaagcgattatcgtgttgaataaattattccaaaaggaaatacatggttttggagaacctgctatagatatatgccaaatttacactagtttagtgggtgcaaaactattatctctgtttctgagtttaataaaaaataaataagcagggcgaatagcagttagcctaagaaggaatggtggccatgtacgtgcttttaagagacgctataataaattgccagctgtgttgctttggtgccgacaggcctaacgtggggtttagcttgacaaagtagcgcctttccgcagcataaataaaggtaggcgggtgcgtcccattattaaaggaaaaagcaaaagctgagattccatagaccacaaaccaccattattggaggacagaacctattccctcacgtgggtcgctagctttaaacctaataagtaaaaacaattaaaagcaggcaggtgtcccttctatattcgcacaacgaggcgacgtggagcatcgacagccgcatccattaattaataaatttgtggacctatacctaactcaaatatttttattatttgctccaatacgctaagagctctggattataaatagtttgaatgcttcgagttatgggtacaagcaacctgtttcctactttgttaac

SEQ ID NO: 58 - DNA, 1915 bp, Oryza sativa
  misc_feature 1..1915: note = Exemplary Oryza sativa sucrose synthase I gene promoter (RSs1)
  source 1..1915: mol_type = unassigned DNA
caatccaccaaatcaaaccgtgagatttttgcagaggcaaaacaagaaaagcatctgctttatttccctcttgctttcttttcatccccaaccagtccttttttcttctgtttatttgtagaagtctaccacctgcagtctattattctacagagaaaaagattgaacctttttttctccaaagctgacaatggtgccggcatatgctaataggatactcccttcgtctagtcccttcgtctaggaaaaaaccaacccactacaattttgaatatatatttattcagatttgttatgcttcctactccttctcagttatggtgagatatttcatagtataataaatttggacatatatttgtccaaattcatcgcattatgaaatgtctcgttcgatctaggttgttatattatgagacggagagagtagattcggttatttttggacagagaaagtactcgcctgtgctagtgacatgattagtgacaccatcagattaaaaaaaacatatgttttgattaaaaaaatggggaatttggggggagcaataatttggggttatccattgctgtttcatcatgtcagctgaaaggccctaccactaaaccaatatctgtactattctaccacctatcagaattcagagcactggggttttgcaactatttattggtccttctggatctcggagaaaccctccattcgtttgctcgtctctgaccaccattgggtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttttttttgaactgagaaaaatctttgggtttttggttggattctttcatggagtcgcgaccttccgtattcttctctttgatctccccgcttgcggattcataatatccggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgagatatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcagttcctgaaattgagtcatg

SEQ ID NO: 59 - DNA, 947 bp, Arabidopsis thaliana
  misc_feature 1..947: note = Exemplary Arabidopsis thaliana sucrose-H+ symporter gene promoter (AtSUC2)
  source 1..947: mol_type = unassigned DNA
agcttgcaaaatagcacaccatttatgtttatattttcaaattatttattacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcttttgaaataagaaaagtgatagatatttttacgctttcgtctgaaaataaaacaataatagtttattagaaaaatgttatcaccgaaaattattctagtgccacttgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaacgtccatatcagtaagatagtttgtgtgaaggtaaaatctaaaagatttaagttccaaaaacagaaaataatatattacgctagaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaaccaccaaaaaccctctcaaagaaatttcttttttttcttactttcttggtttgtcaaag

SEQ ID NO: 60 - DNA, 1321 bp, Arabidopsis thaliana
  misc_feature 1..1321: note = Exemplary Arabidopsis thaliana 5-methylthioadenosine nucleosidase 1 gene promoter (AtMTN1)
  source 1..1321: mol_type = unassigned DNA
cagcgaaaacacctttgatgggagcggtatcaggaggctcttgtccaataaattcgaattcgataaggtaaactaccatacatatatatgttatctagcttttatgctaaaggaaaactttttaaatgatggtaacgagtgatgatgatccggaacggtttggtcgcaggcactaaacgttgccatggagacgattccaaaagaccgtcagggtaaggtgtctaaaggatatctacgagctgtgcttgacactgttgcaccatcggccactttaccaccaataggcgctgtgtcccaggtaaataatgccccgtctaaattattttgtcttttaaattgtttattttgcctttgaatttacatgttacaattatttgttaaacaaatgaaaccagaattagtgttttaatcaaaaattattagtgaatttttatttttattttttgaacggcattgattagttaagtttgtttttgtttataagatggataatatgataatggaagcgttgaagatggtgaatggagatgatggaaatgtggtgaaggaagaagagtttaagaaaacaatggcagagatattggggagtataatgttgcagctcgagggtagtcccatatcggtttcctctaactcggtggttcacgagccgctcacctcggctacctttctgccgtcaacttcgactgatacagaggagccttcaaactaatcatagaagggaataagcagcactagcagcaacaaatgttatatggttttgacttttgagtgtttacccccaaaagttttagattaatgaggaaaaccgtctttactttcagatgtataaaattgaaagtttggggtttcctcttgttggtgtggtgattctactcatgccttttttttttttttctaatgaccatgggatgcaatgtttactctgttttttaatttcgttaaaatttgtttacgtttatgatgcttgaatggctatgatgaaacatttgagttatctttaaaagtgtgaaataaatattctgaagttaattgaagaatttgaaaatttgattacaagagcttggctaaaactacaaggagaccagattagtacaaaaacttagctaaatttaattaattacggtcattagcacaaaaaaataatttgtttttattatattattattggtaagtggaaacacaaaagaggaccaaaaggtccaaaaacgaataaactgtatctctcattcgccggagtttccagccgtttctttccgattctcggatttttcctgggaatcaaacgcatcgccgagaatcggaagagagggataaggtt

SEQ ID NO: 61 - DNA, 3093 bp, Cucumis melo
  misc_feature 1..3093: note = Exemplary Cucumis melo galactinol synthase gene promoter (CmGAS1)
  source 1..3093: mol_type = unassigned DNA
tctagatgacttggattaattctctaacaagaatttagtttaattgacatttgtatgtttgaggactaagaggactttagttttaatttctaatctaatttgtactagaaaagaaaaaaaaagagtcggattaattctctaccattgagtggaggatacttggatgcagttcaagttctcatctctccaatttgtcacgtgacagcggatgattaagcatatgagtaggctgcaaaagattatagacgtagaagatgatacccaatacaaaggcgtaacttttcccggatgacttttatactctttacaaaattggaagtcctattctatctacatcttaatttccagttgttataatgaagaatagtctgaaaatgatatcaattttttctttctcaataccattcaattacgttaagattattaggagctgccattattattattattattgttgttgttattattattattatgcaaccaagtttgatttgaaattgtttgccaaattttactccaatttgatgttgtttaattactttagatggtataataagaatgaagttgaatttaaagaaaagaaacaaagcttgaaagaatggaatacttaggtgtagaagaagacaacgtatttataacgtcgtatagtgtaaataaaaatgcacacatttggatgccctttatgcttcttagaggtcagactttcccacaaaggctaaggtgattcaatcgtgtgggacatcttgttctcccatttgattctcgttttcattagaccaaaattaacaaaaaaatagtaataattctattctttttaaagtttgtgatattacggtttatcctttgttaaaaaagtttatctttgaatgtaagaatttgatagaatgttgaatgaaaattaagattttgaaaagttttgctgaatttcaaataatataactctctaactttggtttaggaaaattaagtgatgacaattatctctattagaattagtattataagtgatatttgagttatgcacttgacttggtcgtgttggtaaattctttggatacagaacaaaagaagttgcatgccaagaaagatttctaatagatatggtgagatatgtggccgttggctctattggattggtggtatgttccagagaagaggagtgcgtatggatacgacctaggtggataaatgattatatgaggagatggtaattttatgaaatgtgttagagctttgatgttaatatatattttttaagtgtgttttgtgatcgatggtattagatgagttccttattaaacatgttttcttggtttttctcgaggtggggttctcaacacttggtaacatgcatcatgtccacgagatgttcttcatcttatctcttgtaatattatatatgatatctcacacaatacaggttcgtctgaaaaatctttctttatttgaaattttttaggtatttattcttgaggatttttttattcttaagtaaagtgttcatgatttgaagttagaaatataggagttatttttaagagagagtctcacactcaaagggagtctaaatatcttttttactaatttaggttgtgtaataaccttgtatttatcgataagtatcacgatgtaatcatttaactatctattaacgaaaatcttttttaggacacgttgcctcctagatagatgcaagttgtattgcaaaacttgtactctgttttttagttttttacatgttttactttagaactaaacctaagttatgttatgtgtcaaataaacttctttaaaataatattaaaacttctcaaaataataggaaaaaaaagaaaaatttcaaatttaatatatatatatatatattgtaatattagctttcattatcattgaattaaaaattgcatatacaagaatcgaataatgtggagaaagtagttttcctttttcaactttgtgtagaggctaagtctctaaaatattggcttcgactttgtacttttggatccgccaccacaatcagacaaacttccatttgatcattacctttatcgaatcaaattctttcccttccaatctgtcacaattttgaacataccatccaccttctgattttttgattctaaataaaccttattagcagagatttttaaaattagtattaaattataccaaataccctaatgaactttttcaatagtttttctattttattttttttttcttttgtgtgtatgagttttttcaccaccattagaaaacacatttgaaatatacagaaccaaattgtttaatttgaattggttttccataccatttttacaaaatacatagtataaccaaaagaactatagttttaagtagtgtataatagtttaattttaaagacaaagaactaaacaataatcattatcaaaaacactaccttaaaacagaattgaaatcaaatccatttgtttaggaatatatatatatatatatatatatataatatagtatcataatatataaaaaaaatgtcaaaatctgagattctttgatcctccctaaattgtccatttttgtcttgcctacaaacttgcaaaaaagaaaaaaaaaaaggttcatagatagaaatgacccataattgaatcataaagcaataaggatatacaaaattattatatccaagagggatgagagataatcttaaaggtgcaaaagaatcttcttattgatggaagaagagaatacaaactcttccaacttttgatcaaaatgcccataatgccctccatctcaccttaaagataggatattccaagtcatattcatcccaccaataccaatatctaaaataataagtaacaaataattacaattacaaatataaagtgcatagaaattaaacttaggggtatctataaacttaaaacaatgttccccaaggctctataaatagcctccttcccatcccttcacaactcaagcttgaaggactaaaacaagaacttgtaagcttgcccttcttattaagtccttcttgcctcccttccttcggagagaaaaaacttttgttgtttcaaaagcaccaaagtcaatatgtctcctgca

SEQ ID NO: 62 - DNA, 887 bp, unidentified
  misc_feature 1..887: note = Exemplary Epipremnum aureum metallothionein promoter (rrEaLeaf1)
  source 1..887: mol_type = unassigned DNA
agctacgctctttgtccacaatgtgacaaggaatgagaacgagtcagcagtagatcatctggcgcgctctctgattggtgcgttcacctcccgtacccatgggcacgcacccgagcaggaccgggcacccccagtgagcccctcacatccatttcctgccctgtcgtggagtgcagtctcttcgacgtccccgccttataattaattacctgtgcgtattcgtccgcacgctactgtgcaacgattccaccataggatatatgaggggcttatgcttatcatatggagttcaaattttcttttttatttttttttattttttaatttttttattcatagttctagttggatttttgatattagagcaggtctttttacaaagatgctatttttgtgaattaaatttacgaatttgtcatctttattttaatataatcataaaaatatgtatgataatataacataaattcatgtgcaacaatgacatatttgtcaaaaaaaaattattaaaataatgattatggaagaggagaagatatagaattaaaaaatcagataggacaagagaagaagataaatcagaactggccatcctttgaattcaagtttgtttttagtttatttaatttttaattaattttatgtggtccgaccacagaaaaagaacaaccctaaatttagccttcaatacattactgtggtgcgaggaagctgcgtccccatatgcccatggcgtgtggagctggtacgactgcttctgtctcgacgtgcgttccccccggaagaaaaagagaaggaagtgacgtgagaggtccagaggcagccgaccttctcctccattatcgggagagattcctctcgggactcccactcgcaagagccctctc

SEQ ID NO: 63 - DNA, 1068 bp, unidentified
  misc_feature 1..1068: note = Exemplary Epipremnum aureum ribulose bisphosphate carboxylase/oxygenase activase 2 promoter (rrEaLeaf2)
  source 1..1068: mol_type = unassigned DNA
ttgttcagaaaggaaccccctagtttgtaattggaggtcataagaggtactttcagtcctcaaaatttatcatttcttaatgaaatttttaattttaaaagatttattctttttaataatttttaggttgagatcaagtaaatttagaagatgattttgacaacgatttttttgaagtagataatcaaaattaggagttttaagaatgataataattattattttaataaaaatttaaactcaccttctataaacagatgtctctcattgtaccaaaaattttagatttacatattattataaaaatatcttttcattttataatttataaaaatattttttaaaattaatttatttcaaaatctatcatgagctgtcttaagataagagttgcataattataattattttttaattgtaataaataaatatccatactaccctcatgttaaaaaaatatatatatatatatataaaatcatccctccccctctctctctcctcgtctcttatgtttctgaatcacatttttttaaaaatattaattaaaaataaaatatttttaaatgttttaagtataataatatctaattaaattttttgaaaacattttttaaattattttataaatgataaaagagatctttttgtagtgccagctcgtaacaaggtatatttacgaataacccttccttttattgcagacacctcggctgagagtacgcagtagatgacgggtcccactttttttccccacgctccaaatagctccaacgtcgtcaggacacgacttatctgaacagaagttatccgccctgattgcgccacgtgttccggcccaatccccactgtgtggccacaggaccctccgctctccccctctcctcccctcccctccgccagccagagggaaaaggaacagaacagggcgatctccagaacctccgcaggccgctttatatatagttcgccctaccccaccgcctccggccaacgctgctacgaggagctgagcttttggtggaagcggcgatcccccccttccgccttctaggtcttccgggtccc

SEQ ID NO: 64 - DNA, 1013 bp, unidentified
  misc_feature 1..1013: note = Exemplary Epipremnum aureum hypothetical polypeptide AQUCO_03600155v1 promoter (rrEaLeaf3)
  source 1..1013: mol_type = unassigned DNA
gtgcgatccctctttccctccacaaattaataaagcctgatttgggttttgatcacagaagatctgtgttgcttgatcgatgtgttgataaagactaaaaagaaaaagaaatcctcgatctattaatttaatttttaaacaataaatttacctattctctttccattcccttcagtcttcatggtttcattaatggcgttatatgcccttgtgagagatttaattgcgtaactatctcttttagatttgcatcttcacgcgcatgtcatcctcatgcggcaatgtacctatctatccctcccgtgagggtatatatacgattaaaagtatcatcaagatatttttaaaatttacagctatacacctcttaatgatataatggcacacacgtttgaaggaagagagtgtatacacacgaatgtaaatttagaaaggatattcatgcaagtgggactctaatagacatgtatggaaaatgtctgtttttttttaacccatatccaattcactcgagtataaatgaaggtgataattatttgcatgtgcttggcctttttaatgtaaatttggtttataccagtggcatgtattcaaacttcctttatttttcggtctgcatccatctccctctctctggtgtcttcttcttcacgcagccagaggttaagggagttgcgtgtgcaagtgcaactgggcaacagtgcaagcatagccaaagggaagaagaaagaagaggaattgacacgagaggtggaggggtagcccccctccttccccaccataattgagattcctttggaagcttcctccatggaggcgtgtgcccatcacacacaggggccctcccctcccctcctctccttgtgccgtgtgcgtccctctgccatccccccctggggcctataaatatcgtcgcagggtggaagcccctccaccatagctggagctgacccctgagctgagagatatatagcagaagctctctttgatcatctctagaggctcccctctgc

SEQ ID NO: 65 - DNA, 1114 bp, unidentified
  misc_feature 1..1114: note = Exemplary Epipremnum aureum carbonic anhydrase 2-like isoform X1 promoter (rrEaLeaf4)
  source 1..1114: mol_type = unassigned DNA
cgcacgtagccttcgttactcatcttgttgttcgtctaatttggagagatggtttcaagcatttgacaatccaaggagacaaagtcattagtattaatgtttctctgttaattaattgtctccctgatatcctgtctcaagtatgtttatgtgtgtgtgtgtgtgtaaatataaatataaagaacaatatgtgataaaggataaccattctgcatggtggatttgtcttcattaattaatatagttctttctttccatcatttgatttcatttcatacactagtactttggtaccatgtttatttttcaaggtttatcgaacaggaattattcagaagatataccaaaaatcgattggattcattctctattcagactgttaattgttaaccatcgatttaaacatgtcatcttaagggaaattaagaaactagattgtgtttacgttttccacactgttagaccttctatagtatcttcattgttctcgagtcgattggtagtattggaacgaactagcatgcatgtgtggaacaccccctcttatatactgcaaaaaatgaaaaagaaaagaaaatggaccatcactttgattttttagggtttggtggcttcaagacacgatgcttggctgggtgcaattaaactgtgccataaaaatgtactatgctattcaataatcgatttcatgagacatggtacatgtcatatttcataaatgacgtggtacatgccaaatttcataagttttcttgtctagaaacttaataaattactattcgcatagaaatcctgaatttttactatttctgatttcccccacccccagaattttaaggttgaagctatcagaaaaacaagaattattatatataatccatctgcaatgcatgagattagcgatacacctgcaacgccatcacctattccatccaacgattacatgacactgtcatctccaagccttctctctctctctctctctccctctcccttatttgaagcagaagccatggttgatccggctttcgctttccttatcctaacccacccccgtcgcagagactatatatcgagccctccacccctcctgggacgggtgtgaaagagagca

SEQ ID NO: 66 - DNA, 907 bp, unidentified
  misc_feature 1..907: note = Exemplary Epipremnum aureum beta-galactosidase promoter (rrEaPetiole1)
  source 1..907: mol_type = unassigned DNA
ttcgatctccccctcgacttgaaaaaactaataaaaaaatgtaaccttatatttttccgtaagtaaaacggaaagtatatttaatagaatataaaaaatctgtaatttaattattattcggataataagagaaagaagaggagggcaaaattatgggagttgatggatggatgatgctgccacgtcagaactcggaccgggacgtggccggccgggtggcgccggtcctgcccgcccactcgctttcaccccacgccctttaaatcccacccggcgccccgtttccctcgccacggccatcaccaccaacggcctctctctctctctctctctctctcgcgatcttcacagccacttctcactccattacgctcttgtttactcctcactcccatctccttaaacgcaagcgactgcaacccaaaccacgctcttccattggcctcgtcctcctctctcgtatcccgaaagcgagagaggaccggccagagaaaggggacagaagaaaaaaaaaagagtcggagggagaaaaagagtgggccgagcgagaggagttggagagaaaattatactgaagagcaccctaaagcgggcaaggaatattgctggggagttgggaggagagaacaaaacgagagaaggaagaaagaaaggaagagggagacgcgcagtgttacaaggaagattaggggataaaaaaagccgttttcttcttctctgctgctgcgaggtcgctgaccgccttccttagactcctctgctggacgcactacttcccatcttatcttagctttctccaacctttagcttctgacacattaaagaggagggaatatagaggagaaaaaaaaaagatcgtcggaaggaagaaaggaaaaaaaaagatccaaccaggtttctgcggaag

SEQ ID NO: 67 - DNA, 928 bp, unidentified
  misc_feature 1..928: note = Exemplary Epipremnum aureum vacuolar-processing enzyme promoter (rrEaPetiole2)
  source 1..928: mol_type = unassigned DNA
tggttgaagtgctaaatttggcattgcctcaattttgttactaagatttttgtaatatcaaaaattaatattataattaatttaacacaaagttgaaataattcagatgatcttgtcaaattattaatactgttgatgatattacactatttaataaaagaaccatatgccccataaaattaactcggccttcactgaagaatgatcaagtggtcattatgtaatcatctgaaactcagggatgatacatacacatacatgtctaaaactcctagaaactgtagttaattgcacccttttgccactgcattatttcatctggtaccaactgacatggcatcccctgtccacttgctattggatcaacacgcccgacttcttacgtcgccacgccggggcccacctagataggaactatctgcttgatcccgtcgaatcagcagcgttccaagcccgctcccccatcggatagatattaaccgtcggatcaatggatccatcgtgggaacatctatcttccaatgccgaacagcacaactaactcccaaccgccaccgctggcccacccaccgatcgttgagccggatcaggatcctgcggccctcacgtgacccccagagaacatcgcctcctcataggccgtcgcgtgcgagggctgacgcccgtcaacacgacccccagggaagacgtcacgtcggcaattccggagattcaaggcgagcgcataggccgcgccaattaagctaaaacccgaagaaatccttcgagcagagcaacagctcggcggggccccactttttctaactttcccccgctccagtctataaatagcgcccactttccgcccaggtttcctcgccattgacgattagagcactcgacggaggtaaagctgcttccctgggtgccccccgcaccaccaccaacg

SEQ ID NO: 68 - DNA, 944 bp, unidentified
  misc_feature 1..944: note = Exemplary Epipremnum aureum cathepsin B promoter (rrEaPetiole3)
  source 1..944: mol_type = unassigned DNA
ctgaggaaccccattgcagttttactacggtcagattggaggagagatcgaggcggcacacgtaacggcaaaacgtcacgttgacggggctcttatggttcccgtgttacgtaaacccccggcattgggaccattgggactcaccaagtcccgtgtgcgattgtctctcgagtggcgtgcctcatcactcaacacaagggcgaggggtgcacggcgctgtcgtcaccccttacgtgagcacgcggtataacgataacggcatctaccatccgacgggaaggaacagcgtcagatcgtagcgggatggaccgtcacggcctcctatatatctgatgaagcgccgtcagatcgggagccctgggcccacagcattggggtgcaaaccaatcaaatgccacttcctccaataatggacactatgggttccagcttcgaagaagcggcagctggcgcctccgtagctctctctctctctctctcaaacggcggcgtcatcttatcctatcgccttttcagagcccggctgcgcaagtaaccgtcccgttgatttagatctggatttcatttatttgctacgttgaaatcagggtccaatcgcactgccatcacccccaaacgtccggattccatttatgttatacgctgaatcgaggttcagccgcgttgccatcaccgtcgaaataggtaccgccgccgccaagcttccatatcatcttccccctcatatcaaattctgacccctctctctctcgccccccttccttcctggtcttgctactccgctccgtccctctccccgtttcacctctccacctgctgtctgtaaatggtgggggtgctgtttcgagctgaagggtgagggtgtgggggtgctgtttggagcggaacggagaggatagggcacagatatagctagggggagagagagagagagaacaacgggg

SEQ ID NO: 69 - DNA, 1055 bp, unidentified
  misc_feature 1..1055: note = Exemplary Epipremnum aureum metallothionein-like protein type 2 promoter (rrEaPetiole4)
  source 1..1055: mol_type = unassigned DNA
gtacgcaggctgaaagaagcctctttattcaattgagaagtgatagtaactattatccaatagagtagggagaagacgtatacatccttttctatggcatcgtttactttgtctgtccaccatgaatgtactctataataagtagtaatcaatgaaatgataccttaaaaaattagatgtttgtaatggcccccccttagtaatcttcctagtgacggatgcactttaaaatattggagaaaaaaatgatggttgcagtacaacaatatcatattaggtaagaaaaatacaagagtgtgtggagacttggtctacttttgatgtaaaaaaactgtaaatattgatgggttgagttagtattataaaaaaagaataagtttgagtaattccttttcacatagaaaccttttaagtccctttcatatatcaagcagcagacaagaatttaaaattttgaggtcttcacatgttggatgcagtgctcttctaattagctgtggcggcaggagttcatgaaaattaagaaaaaaatgatatgaaaaatgacaagattccctacttcatccgacaatgcatatggtctggggcaaattagaataccacacttctctcgtcattctgtcattactcctttttttattttaaaaaactcacctcatcatttatagtaccgcatgttaactcaggtgttatttgataacgttatcagcgttgattttatcttttaatttttataaaattttaaaaaatatataaatattactatcaaatgaataaatactaaatcagatttaaaaaataatttataattattagattaaaaatcactttaattcattttaataaaatctaagacaatcataatattgatatgatttaaaatttaataagaataacataacgataatattatcaaatgaagtgtttcaaagatcacaagttatcccatgttcgcaagaagggtaatataactgttgacggcacaactattgtaggagttttaaataaagatctatataacttgacatgacgtgaggtagcagagaccatcaaga

SEQ ID NO: 70 - DNA, 926 bp, unidentified
  misc_feature 1..926: note = Exemplary Epipremnum aureum metallothionein promoter (rrEaStem1)
  source 1..926: mol_type = unassigned DNA
cccgatgagcacctcagatgtccatttgatgctctttcgtgaagtggattctctttgacgtacacatcttataaatatctatattcgtccacaccgctgtgcaacgattccctatgtgatatatgctgcacggacggagagggcggttgcctgaaggaacacatatgcttatgtggagcccagttctctttatacttttagttggctttgatttagttttttttttttttttttgaagtaggagcagatcctgtgttgttgcagatttactacctcggctgccacccatagaacaagatcatattaatctgtctcttggagctgaaatatggggagcaaagaaagggtattagaaagattcttaaaattagtagacctgtcctaagacactggtgattgagcagtggcatctgcacttgtggactgtgtgcttgtgcatggacgctggctggagagatccgccgacgtgcatggcgagggtgcatcaataggactggacaagggaagaagaaacatctgaactgagtatcatgtgaaattaaaactttttaataattttattttattttaaattaattttatgtggtccgaccacaaaaaaaacttacagaacattactgtggtgtgaagaagctccgtcgccatgctactggcgtgtggggtcggtaagattgtctctgcctcgacatgtgttcccccctacagaagaaaaagagaagaagtgacttgagtggtcgagacgcagccacccgtctcctccattatcgagagggattcctctggggaatcccactcgcaagagccccagcaatgcctataaataccggtggaggcggcccctctccagctcacacagagccgacgtgataagctcctcctctcgcttcagcagttctctcttgccttcgccacttcccattatcgcc

SEQ ID NO: 71 - DNA, 1045 bp, unidentified
  misc_feature 1..1045: note = Exemplary Epipremnum aureum dormancy-associated protein 1 promoter (rrEaStem2)
  source 1..1045: mol_type = unassigned DNA
tgtgagtgaccaagtgtgcttaagagcaaccaaagactttggtgagcatcatagtgcattatgttacccatcaaatatcatattgctcatcaaaagttactctgtggatagcacaacctaccatgttactcatatagaggtgtctagtgaataacaggatgttttgatggataacataatacatcatactacttactaatacatttagttgttcacaaagtatcacattatttattcatcaacacattaagttacttatgggcatataaaattacttaaagtatcccaattactgaggaaagatttagatgtataatatttttaacttatttctagtacaaatggggtgcacaaatagtgaacagagtgaggtcattttctgacaattccattgggtaatttttttttactctcttttttctttcaaactgattcaaagagtttaatggtgacagagtcacatatctagaagaatattattgggggcgggtgcaatgttgtttgcactacaagtcgacgaccggtcgtcacgtggatcccatagtgggccaggtccatgctatgataaagcccatcaaagggcagatatttccgtcgtcacgtgatggagggggggcccaaatcgtcttcatgcttatccgctacctgtccataccgccatcacgtcactctcccacagctttgatcacttccgccccctcccgcccagctaccctcgagacccggtattcggacgtcttctcggatccgaaatatccgctgttatctcgggttttcttgttggagtctcatcctccccttcacttgagacgatccggactcgatcagagtgttaaaggatggggatggagacgtgtgagtgagggcaaaaggaaacctacgtacaggttgtctgaaggaaactttttccagcactatcctgctctcgttacctgtgactatccgttaatttggcatctgagcagaatctctttctatatatggagttggcgagggcagcagcaataggggtgcagagccagtgtagttgtggttgagaaggaag

SEQ ID NO: 72 - DNA, 1078 bp, unidentified
  misc_feature 1..1078: note = Exemplary Epipremnum aureum dehydrin COR410-like promoter (rrEaStem3)
  source 1..1078: mol_type = unassigned DNA
ctgaggacgcttcgagatccactgaccatgccactttttttttacgtgaacgaggcaagtcggcattgacgagcggggatgaaaagggccgtggagcgaaggggacacgcacgctcataatactgttctgtacggcttatatagtataaacagatccagcgcagcgcccgcgcatgtggcggggtattgggggaggcgatggcgcgcgtctgctcccccgccgtgaggccaaggacctccggtaggggcgcaccgctcgcggtgtatggcggccgtaccgtggacatgcatgtatggtgggctttttttaagtttgccccggataagtgttactgttgtggacatgcacatgcatacgatgatggggtccgtctgggtccgttgctctactcatccgatgccacgcaagctctgtagtaaatgtatgtatatattcgtgtgagaaagaggaacgaaaagggacaactaagcgaagtccgatggctcatcttaatgattaaattacaaaaaaaaattatttagatatcttcgtatcaagtctctagagaataatctgtcatttaaagtttgaggttattttatggatatttctttctcctttaatgacttataaatattagattttacttctctcagttataaaatcactcatcattccaactgagttatttatctaagatttgatgacaaggggaagacgattacgatgggcgctctccaagcgttgctgtggaatttctcgcggtgagtggcgatgacacgtgaaactttgtcacaactactccaagaatcccactagccattagcttgtatgatattaatactgagactggttattaacaaacatctaacaccaccttttatttaccagacgaggacggtaacggaaaacaggggaatgaaagcaagagaaagccgacatcggaccgacgttcctcgaggcccgatctgatccactccaacccgccatcgtcagcatcaccgtctcaaatcaagtccatttatcgcccgctgcgaaagggaaaggcaaagggtttgaaaaaaaaaaagaaaggcaacgaaagggggacgaaggtgg

SEQ ID NO: 73 - DNA, 899 bp, unidentified
  misc_feature 1..899: note = Exemplary Epipremnum aureum ubiquitin-conjugating enzyme E2 8 promoter (rrEaStem4)
  source 1..899: mol_type = unassigned DNA
acatgacactaggcaggatcattcaatacaactaacttgaaagataatgaaagaaaataacaataagtgattacagtgttagcattaattattttttattatcttcatcttttgtcccactagtattaaatacttaaaaaatgtttaaattatatgcgatcactaagatgagggggagaggggggtatgagtaactaaaaacatctttatattataaaaagtagtgcaataaatatcactctatttatatgtaagggcaaatgtacaaataagagagattctaggggctgcctccacaaaagtcccttaaacttgaagatcccttctaagttttaagatttaacattctttttgttgaactaacgcaattccactgaggtttaattcagattttacttaactaaattaaatatttaaaaaatattatattttaaatttataaaaatatataaattattttaaatattatattattttttaaattatttataataatttagataatcctcaacaaaccatggttagaagttcgaagttcaaacctgtgccctaccgttaccaccgtgtggttgcctgcgacctgttcgaaccggattcctctttatatatcctttaaatatattagcgccgctcctctctctctctctgtctctctcgccgacggcagcctctgtccccttctacgggtcctcgaggaggggcggggcgggcggagggggtcggtcgcacgcagcaggcagaagagagaagcattccaccgcgctctcttccgcgtccgttccctccctctccgcctccgtttgttccctgctttcctctcaaccctgacggtttcctctcttctttcccctctctatctagggtttcggagagattggcacgtaccgaccggggtttcc

SEQ ID NO: 74 - DNA, 2041 bp, unidentified
  misc_feature 1..2041: note = Exemplary Promoter PVUBI2
  source 1..2041: mol_type = unassigned DNA
ttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggt

SEQ ID NO: 75 - DNA, 2513 bp, unidentified
  misc_feature 1..2513: note = Promoter BdUbi10
  source 1..2513: mol_type = unassigned DNA
ttcaagcttggaggaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 76 - DNA, 209 bp, Cauliflower mosaic virus
  misc_feature 1..209: note = Cauliflower Mosaic virus 35S terminator (TerCaMV35S)
  source 1..209: mol_type = unassigned DNA
agcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 77 - DNA, 490 bp, Arabidopsis thaliana
  misc_feature 1..490: note = Arabidopsis thaliana Actin 2 terminator (TerAthAct2)
  source 1..490: mol_type = unassigned DNA
agcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtgttccattcacaaatgaacttaaactagcacaaagtcagatattttaagatcgcaccattt

SEQ ID NO: 78 - DNA, 281 bp, Solanum lycopersicum
  misc_feature 1..281: note = Solanum lycopersicum Histone H4 terminator (TerSlHisH4)
  source 1..281: mol_type = unassigned DNA
agcttttatgttggtgatatggtggtaaatgtagggatttagtttacaattgcgtatgtctgtgttggatatctgtagtgctgttcttatggcttagatcttgtaatttctcattacagtatcaatgaatagatatcagtttctagtgatgacattggttcgtcttttagctgttgattaatttttcttaattgattcatcctattgcaattcttctgaatttaaattgtatactgtgaaattaagaaaattcttgaaattaatgagaatttgagtaatag

SEQ ID NO: 79 - DNA, 209 bp, unidentified; identical to SEQ ID NO: 76
  misc_feature 1..209: note = Agrobacterium tumefaciens nopaline synthase terminator (TerNos)
  source 1..209: mol_type = unassigned DNA

SEQ ID NO: 80 - DNA, 735 bp, unidentified
  misc_feature 1..735: note = Agrobacterium tumefaciens octopine synthase terminator (TerOcs)
  source 1..735: mol_type = unassigned DNA
agcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgcttgagaccacgaa

SEQ ID NO: 81 - DNA, 255 bp, unidentified
  misc_feature 1..255: note = Agrobacterium tumefaciens mannopine synthase terminator (TerMas)
  source 1..255: mol_type = unassigned DNA
agcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatc

SEQ ID NO: 82 - DNA, 406 bp, unidentified
  misc_feature 1..406: note = Agrobacterium tumefaciens agropine synthase terminator (TerAgs)
  source 1..406: mol_type = unassigned DNA
gaattaacagaggtggatggacagacccgttcttacaccggactgggcgcgggataggatattcagattgggatgggattgagcttaaagccggcgctgacaccatgctcaaggtaggcaatgtcctcagcgtcgagcccggcatctatgtcgagggcattggtggagcgcgcttcggggataccgtgcttgtaactgagagcggatatgaggccctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgat

SEQ ID NO: 83 - DNA, 708 bp, unidentified
  misc_feature 1..708: note = Terminator OCS
  source 1..708: mol_type = unassigned DNA
tgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctactagaatt

SEQ ID NO: 84 - DNA, 284 bp, Arabidopsis thaliana
  misc_feature 1..284: note = Arabidopsis thaliana DEMI intronic nucleotide sequence.
  source 1..284: mol_type = unassigned DNA
gtaagcagaactctagttgcagtgtatattcttgctgagaaagtgacattcttgaaattttcatgttttgctcatagcataagtgcatataatattgaagtcttaagaatttttgtggaaattgaattatagtgttcctcagttgccttgtgtttcaaccttgatttttgatagaggaacttttactactgttgaatcattcatcaattgaaataactttttactaatagttgattcctgactctttttgtctatcttttcttgttgaaaatgtcgatatatag

SEQ ID NO: 85 - DNA, 67 bp, Tobacco mosaic virus
  misc_feature 1..67: note = Exemplary Tobacco Mosaic Virus (TMV) 5 -leader sequence (Omega).
  source 1..67: mol_type = unassigned DNA
gtatttttacaacaattaccaacaacaacaaacaacaaacaacattacaattactatttacaattac

SEQ ID NO: 86 - DNA, 58 bp, Arabidopsis thaliana
  misc_feature 1..58: note = Exemplary Arabidopsis thaliana Alcohol Dehydrogenase 5 UTR.
  source 1..58: mol_type = unassigned DNA
tacatcacaatcacacaaaactaacaaaagatcaaaagcaagttcttcactgttgata

SEQ ID NO: 87 - DNA, 92 bp, Nicotiana tabacum
  misc_feature 1..92: note = Exemplary Nicotiana tabacum Alcohol Dehydrogenase 5 UTR.
  source 1..92: mol_type = unassigned DNA
gtctatttctcagtattcagaaacaacaaaagttcttctctacataaaattttcctattttagtgatcagtgaaggaaatcaagaaaaataa

SEQ ID NO: 88 - DNA, 101 bp, Oryza sativa
  misc_feature 1..101: note = Exemplary Oryza sativa Alcohol Dehydrogense 5 UTR.
  source 1..101: mol_type = unassigned DNA
gaattccaagcaacgaactgcgagtgattcaagaaaaaagaaaacctgagctttcgatctctacggagtggtttcttgttctttgaaaaagagggggatta

SEQ ID NO: 89 - DNA, 63 bp, unidentified
  misc_feature 1..63: note = Exemplary Cleavage signal nucleotide sequence
  source 1..63: mol_type = unassigned DNA
ggctctggcgaaggcagaggcagcctgcttacatgtggcgacgtggaagagaaccccggacct

SEQ ID NO: 90 - DNA, 72 bp, unidentified
  misc_feature 1..72: note = Exemplary Cleavage signal nucleotide sequence
  source 1..72: mol_type = unassigned DNA
gccccggtgaagcagaccctgaacttcgacctgctgaagctggcgggcgacgtggagagcaacccgggcccc

SEQ ID NO: 91 - DNA, 675 bp, unidentified
  misc_feature 1..675: note = Exemplary IntF2A nucleotide sequence
  source 1..675: mol_type = unassigned DNA
tgtctatcctttggaacagagatattgacagtggaatatggcccgttaccaataggcaaaatcgtgtcagaagagatcaattgctcagtctattctgttgatcctgagggtagagtttatacacaagccattgcgcaatggcatgatagaggcgaacaagaagtcttggaatatgaattagaggacgggagcgtcattagggcaacaagtgatcataggtttcttactacagattatcaacttctcgccattgaggaaatttttgcccgacagctagatctcctgacactcgaaaatattaaacaaaccgaggaagcgttggataatcatcgcctcccgtttcctctcctagatgcagggacaattaagatggttaaagtgattgggaggagatcacttggtgtgcaaaggatttttgatatagggctccctcaggaccacaacttcttactggctaacggggcaatcgcggcagcttgttcatgtggtagtgggtcacgggtaactgagttactttataggatgaagcgagctgaaacctattgcccaagaccccttttggcgattcatcctacagaagcacgccacaaacaaaaaattgtggccccagttaaacaacttctcaattttgaccttttgaagttggccggtgacgtcgaatctaaccccggccct

SEQ ID NO: 92 - DNA, 65 bp, unidentified
  misc_feature 1..65: note = Self-Cleaving 2A Peptide P2A - 4 polynucleotide sequence 1
  source 1..65: mol_type = unassigned DNA
ggtagcggagctaccaattttagcctccttaagcaggcaggtgatgtagaagagaaccccgggcc

SEQ ID NO: 93 - DNA, 66 bp, unidentified
  misc_feature 1..66: note = Self-Cleaving 2A Peptide P2A 4 polynucleotide sequence 2
  source 1..66: mol_type = unassigned DNA
ggatccggagcaaccaactttagcctgctcaagcaagcaggagatgttgaggaaaatcctggcccc

SEQ ID NO: 94 - protein, 54 aa, unidentified
  REGION 1..54: note = Exemplary Chloroplast localization signal amino acid sequence
ASSMLSSAAVVTSPAQATMVAPFTGLKSSASFPVTRKANNDITSITSNGGRVSC

SEQ ID NO: 95 - protein, 51 aa, unidentified
  REGION 1..51: note = Exemplary Mitochondria localization signal amino acid sequence
MAMAVFRREGRRLLPSIAARPIAAIRSPLSSDQEEGLLGVRSISTQVVRNR

SEQ ID NO: 96 - protein, 49 aa, unidentified
  REGION 1..49: note = Exemplary Peroxisome localization signal amino acid sequence
MEKAIERQRVLLEHLRPSSSSSHNYEASLSASACLAGDSAAYQRTSLYG

SEQ ID NO: 97 - protein, 21 aa, unidentified
  REGION 1..21: note = Exemplary Cleavage signal amino acid sequence
GSGEGRGSLLTCGDVEENPGP

SEQ ID NO: 98 - protein, 24 aa, unidentified
  REGION 1..24: note = Exemplary Cleavage signal amino acid sequence
APVKQTLNFDLLKLAGDVESNPGP

SEQ ID NO: 99 - protein, 225 aa, unidentified
  REGION 1..225: note = Exemplary IntF2A amino acid sequence
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKMVKVIGRRSLGVQRIFDIGLPQDHNFLLANGAIAAACSCGSGSRVTELLYRMKRAETYCPRPLLAIHPTEARHKQKIVAPVKQLLNFDLLKLAGDVESNPGP

SEQ ID NO: 100 - DNA, 1003 bp, unidentified
  misc_feature 1..1003: note = Insulator Sequence TM2MARs
  source 1..1003: mol_type = unassigned DNA
atcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcgacgctttactgagaccgaggatgcacat

SEQ ID NO: 101 - DNA, 717 bp, unidentified
  misc_feature 1..717: note = Exemplary eGFP reporter nucleotide sequence
  source 1..717: mol_type = unassigned DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 102 - DNA, 711 bp, unidentified
  misc_feature 1..711: note = Exemplary mCherry reporter nucleotide sequence
  source 1..711: mol_type = unassigned DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 103 - DNA, 714 bp, unidentified
  misc_feature 1..714: note = Exemplary mRuby reporter nucleotide sequence
  source 1..714: mol_type = unassigned DNA
atggtgtcaaaaggtgaggagctaatcaaagagaacatgcgaatgaaagtggtcatggaagggagcgtaaacggccaccagttcaaatgcacaggcgagggcgagggcaacccatacatgggtacgcagaccatgaggataaaagtaatcgagggtggtccgttgccattcgccttcgacatcctggcaacctcgttcatgtacgggagtcgaacattcatcaaatacccaaaaggtataccggacttcttcaaacagagtttcccggaaggtttcacctgggagcgggtcacaaggtacgaggacggtggtgtcgtgacagtaatgcaggacacatccttagaggacggttgcctggtctaccacgtccaggtgcgtggcgtcaacttcccctcaaacggcccagtaatgcagaagaaaaccaaaggttgggagccgaacacagagatgatgtacccggcggacggtggcctgcgtggttacacacacatggcattaaaagtggacggtggtggtcacctctcgtgctcgttcgtcacaacctaccgaagcaagaaaacggtcgggaacatcaaaatgccgggtatacacgcagtcgaccaccgtctcgagcgtttagaggagagcgacaacgagatgttcgtcgtgcagcgagagcacgcagtggccaaattcgcgggtctaggcggcgggatggacgagttatacaaatga

SEQ ID NO: 104 - DNA, 702 bp, unidentified
  misc_feature 1..702: note = Exemplary RRvT reporter nucleotide sequence
  source 1..702: mol_type = unassigned DNA
atggtatcaaaaggggaagaggtgatcaaagagttcatgcgtttcaaagtacgaatggaaggttccatgaacgggcacgagttcgagatagagggtgagggtgagggtaggccatacgagggcacacagacggccaaactgaaagtaaccaaaggtggcccactcccattcgcgtgggacatcttgagtccacagttcatgtacggtagcaaagcctacgtcaaacacccggccgacataccagactacaagaaactaagtttcccagaggggttcaaatgggagcgagtaatgaacttcgaggacggcggcctggtcacggtgacccaggactcgagtttacaggacggtaccttgatatacaacgtcaaaatgcggggtacaaactttcccccagacggccccgtaatgcagaagaaaacaatgggttgggaagcaagcacagagcgtttgtacccaagggacggtgtgctaaaaggtgagatccaccaggcactaaaattaaaagacggcggtcactacctagtcgagttcaaaaccatatacatggcgaagaaacccgtgcagctcccaggttactactacgtagacaccaaattagacatcacgtcgcacaacgaggactacacgatcgtcgagcagtacgagcgtagcgagggtcgacaccacctcttcctatacggtatggacgagctctacaaa

SEQ ID NO: 105 - DNA, 711 bp, unidentified
  misc_feature 1..711: note = Exemplary mTFP1 reporter nucleotide sequence
  source 1..711: mol_type = unassigned DNA
atggtcagtaaaggtgaggagacgacgatgggtgtcataaaaccagacatgaaaataaaactgaaaatggaaggtaacgtcaacggccacgcattcgtaatcgagggtgagggtgaggggaaaccatacgacgggacgaacaccataaacctggaagtgaaagagggtgccccactaccattctcatacgacatcctgacaaccgcgttcgcctacggtaacagggcattcaccaaataccccgacgacatcccaaactacttcaaacagtcattcccagagggttacagttgggagaggacaatgacattcgaggacaaagggatcgtgaaagtgaaaagcgacatcagcatggaagaggactccttcatctacgagatccacttgaaaggtgagaacttcccacccaacggtcccgtaatgcagaagaaaacaaccggttgggacgcatcaaccgagcggatgtacgtaagggacggcgtcttaaaaggtgacgtgaaacacaaactgctgttggaaggtggtgggcaccacagggtcgacttcaaaaccatataccgagcaaagaaagccgtgaaattgccagactaccacttcgtcgaccaccggatagagatactaaaccacgacaaagactacaacaaagtaaccgtgtacgagagtgccgtagcgcgaaactccacagacggcatggacgagctgtacaaatga

SEQ ID NO: 106 - DNA, 696 bp, unidentified
  misc_feature 1..696: note = Exemplary RFP611 reporter nucleotide sequence
  source 1..696: mol_type = unassigned DNA
atgaactcattaatcaaagagaacatgcgtatgatggtggtcatggaaggctcggtcaacggttaccagttcaaatgcacaggtgagggtgacggtaacccatacatgggtacccagacaatgcgtatcaaagtggtagagggcggtccattgcccttcgcgttcgacgtactggcaaccagtttcatgtacggttcaaagacgttcatcaaacacaccaaaggtatacccgacttcttcaaacagtcattcccagagggtttcacatgggagcgggtgacgaggtacgaggacggtggtgtcatcaccgtgatgcaggacacatcgctcgaggacggctgcttggtgtaccacgccaaagtgacgggcgtcaacttccccagtaacggtgcagtcatgcagaagaaaacgaaagggtgggagccaaacacggagatgttataccccgccgacggcggtctgcgaggttacagtcagatggccctgaacgtggacggggggggttacttgtcgtgctccttcgagacaacgtacaggagtaagaaaacggtagagaacttcaaaatgccaggcttccacttcgtcgaccaccgtttggagcgtctcgaggagagtgacaaagagatgttcgtggtccagcacgagcacgccgtggcaaaattctgcgatctcccatcaaaactcggtaggctgtag

SEQ ID NO: 107 - DNA, 723 bp, unidentified
  misc_feature 1..723: note = Exemplary dTFP0.2 reporter nucleotide sequence
  source 1..723: mol_type = unassigned DNA
atggtgtcgaaaggtgaggagacgactatgggcgtgatcaaaccagacatgaaaatcaaactgaaaatggaaggtaacgtcaacggtcacgcattcgtaatcgagggtgaaggggaaggcaaaccatacgacggtacaaacacagtcaacttggaagtcaaagagggcgcaccactgccgttcagttacgacatcctcagtaacgcattccagtacggtaaccgtgcattcacaaaataccccgacgacatcgcaaactacttcaaacagtcattcccagagggttacagctgggagcggacaatgacattcgaggacaaagggatcgtaaaagtgaaaagtgacatatcaatggaagaggactcattcatctacgagataaggttaaaagggaagaacttcccaccaaacggtccagtgatgcagaagaaaacactcaaatgggagccatcaaccgagatcctctacgtgcgtgacggtgtcttggtgggtgacatctcacacagtttgctgctcgagggtggcggtcactaccggtgcgacttcaaaaccatctacaaagccaagaaagtagtcaaactgcccgactaccacttcgtcgaccacaggatagagatcttgaaccacgacaaagactacaacaaagtcacattgtacgagaacgcagtggcccgatacagcctgttaccaccacaggccgggatggacgagttgtacaaatga

SEQ ID NO: 108 - DNA, 699 bp, unidentified
  misc_feature 1..699: note = Exemplary meffCFP reporter nucleotide sequence
  source 1..699: mol_type = unassigned DNA
atggcattgagcaaacagtccctacccagcgacatgaaattgatctaccacatggacgggaacgtgaacggtcactccttcgtcataaaaggcgagggtgagggtaaaccatacgagggcacacacacaataaaactgcaggtagtcgagggtagtccgctgccgttcagcgccgacatactgtcaaccgtattccagtacggtaaccgatgcttcacaaaatacccaccaaacatagtggactacttcaagaactcatgctccggtggtggctacaaattcgggcgttcattcctatacgaggacggcgcggtctgcacagcaagtggtgacataacactcagtgcagacaagaaatcattcgagcacaaatcgaaattcctgggcgtgaacttcccagcagacggcccggtgatgaagaaagagacaacaaactgggagccatcatgcgagaaaatgacgcccaacggcatgacgttgatcggggacgtcacaggcttcttattaaaagaggacgggaaacggtacaaatgccagttccacaccttccacgacgccaaagacaaaagcaagaagatgccgatgccagacttccacttcgtgcagcacaaaatagagcggaaagacctgccaggttcaatgcagacatggcgactgacagagcacgcagccgcgtgcaaaacgtgcttcaccgagtga

SEQ ID NO: 109 - DNA, 717 bp, unidentified
  misc_feature 1..717: note = Exemplary Folding Reporter GFP reporter nucleotide sequence
  source 1..717: mol_type = unassigned DNA
atgagtaaaggtgaggaactgttcacaggcgttgtaccgatcctggtggagttagacggcgacgtgaacggtcacaaattctcagtcagtggtgagggtgagggcgacgccacatacggtaaattgacactgaaattcatatgcacaacaggtaaattgcccgtaccctggccaacgttggtaacaaccctaacgtacggtgtccagtgcttctcgcgatacccagaccacatgaaacgtcacgacttcttcaaaagcgcgatgccagagggttacgtccaggagcgaacaatatcattcaaagacgacggtaactacaaaacaagggcagaggtgaaattcgagggtgacacattagtcaaccgaatagagttaaaaggtatcgacttcaaagaggacggtaacatactaggtcacaaactcgagtacaactacaactcccacaacgtctacataacagcggacaaacagaagaacggtatcaaagcaaacttcaaaatcaggcacaacatcgaggacggctcagtgcagctcgcggaccactaccagcagaacacacccatcggtgacggtccggtcttactccccgacaaccactacctatcaacgcagtccgccctgagtaaagacccaaacgagaaacgtgaccacatggtcctactcgagttcgtaacagcagcggggataacccacggtatggacgagttatacaaatga

SEQ ID NO: 110 - DNA, 681 bp, unidentified
  misc_feature 1..681: note = Exemplary ccalOFP1 reporter nucleotide sequence
  source 1..681: mol_type = unassigned DNA
atgtccctctcgaaacaagtattaccaagagacgttaaaatgcgattccacatggacggttgcgtgaacggccactcattcacgatagaaggagagggtaccgggaaaccgtacgagggtaagaaaacgttgaaactcagggtgacaaaaggtggtccgctaccgttcgccttcgacatcctgtcggcgaccttcacgtacggcaacaggtgcttctgcgactacccagaggagatgcccgactacttcaaacagagtttaccagagggttacagctgggagaggacgatgatgtacgaggacggtgcatgctcaacagcgagtgcccacatcagtttggacaaagactgcttcatccacaacagtacattccacggtgtgaacttcccagcgaacggcccagtcatgcagaagaaggcgatgaactgggagccgagctcagagttaataaccccatgcgacgggatcttgaaaggcgacgtaacgatgttcttactacaagagggtggtcaccgtcacaaatgccagttcacaacttcctacaaagcccacaaagcggtcaaaatcccgccaaaccacatcatcgagcacaggttggtacgtaaagaggtgggtgacgcagtccagatccaggagcacgcagtggcgaaacacttcacagtccagataaaagaggcgtga

SEQ ID NO: 111 - DNA, 696 bp, unidentified
  misc_feature 1..696: note = Exemplary tdKatushka2 reporter nucleotide sequence
  source 1..696: mol_type = unassigned DNA
atgtcagagttgataaaagagaacatgcacatgaaattatacatggaaggtaccgtaaacaaccaccacttcaaatgcacctcagagggagagggtaaaccgtacgagggtacacagacaatgaaaatcaaagtggtcgagggtggtcccctaccattcgcgttcgacatcctggccaccagtttcatgtacggctcaaagacgttcataaaccacacacaggggatacccgacttcttcaaacagtcattcccagagggcttcacctgggagcgaatcacaacatacgaggacggcggtgtgttgacagcaacgcaggacacatccctgcagaacggttgcataatatacaacgttaaaataaacggtgtcaacttcccatcgaacgggagtgtgatgcagaagaaaaccttaggttgggaagccaacaccgagatgttgtaccccgccgacggcggcctacggggacacagtcagatggccttaaaactagtgggtggtggttacctacactgcagtttcaaaacaacctaccgtagcaagaaaccagcgaagaacctcaaaatgccaggtttccacttcgtggaccaccgtctcgagaggatcaaagaggcggacaaagagacatacgtggagcagcacgagatggcggtcgcgaaatactgcgacctaccatccaaactaggtcaccgttag

SEQ ID NO: 112 - DNA, 1035 bp, unidentified
  misc_feature 1..1035: note = Exemplary vsfGFP-0 reporter nucleotide sequence
  source 1..1035: mol_type = unassigned DNA
atgtctaaaggagaggagttgttcactggtgtcgtgccgatcctggtcgagctcgacggtgacgtcaacgggcacaaattctcagtccgaggtgagggcgagggtgacgcaacaaacggtaaattgacactgaaattcatctgcacgacgggtaaattaccggtaccgtggccaacattggtgacgacactgacatacggtgtgcagtgcttcagccgataccccgaccacatgaaacgacacgacttcttcaaatcagcaatgccagagggttacgtacaggagaggacgatcagcttcaaagacgacggcacctacaaaacccgtgcggaagtgaaattcgagggtgacaccttggtcaaccgaatcgagttgaaaggtatcgacttcaaagaggacggtaacatattaggtcacaaattggagtacaacttcaacagtcacaacgtctacatcacagccgacaaacagaagaacggtatcaaagccaacttcaaaatccgtcacaacgtagaggacggctccgtgcagctagcggaccactaccagcagaacacgccaatcggggacggccccgtactgctgccagacaaccactacctatcaacacagagcgtgctctcaaaagacccaaacgagaaacgggaccacatggtgttgttggagttcgtaacggcggcaggtatagcgcaggtgcagttggtagagtcaggtggggcattggtacagccaggtggttcactgcggttatcatgcgcagcatcaggtttcccggtaaacaggtactccatgcgatggtaccggcaggcaccgggtaaagagagggagtgggtggcgggtatgtccagtgcgggtgacaggtcgtcgtacgaggactcagtcaaaggtaggttcaccataagtagggacgacgcacgaaacaccgtgtacctgcagatgaacagtctaaaaccagaggacacagcggtgtactactgcaacgtcaacgtaggtttcgagtactggggtcagggtacgcaggtgacagtgtcgtga

SEQ ID NO: 113 - DNA, 660 bp, unidentified
  misc_feature 1..660: note = Exemplary eYGFPuv reporter nucleotide sequence
  source 1..660: mol_type = unassigned DNA
atgaccacattcaaaatcgagagtaggatccacggtaacttgaacggcgagaaattcgagctagtaggcggtggtgtaggggaagagggaaggctcgagatcgagatgaaaacaaaagacaaaccgttagcattctcgccattcctgttgacaacgtgcatgggttacggtttctaccacttcgcttccttcccgaaaggtataaagaacatatacttgcacgcagccacgaacggcggctacaccaacacacgtaaagagatatacgaggacggtggtatactggaagtcaacttcaggtacacgtacgagttcaacaaaatcatcggcgacgtggagtgcataggtcacggcttcccctcgcagtccccaatcttcaaagacacaatagtcaaatcgtgcccaacggtggacttaatgctgccaatgagcgggaacataatcgcctcatcctacgcatacgcattccagctcaaagacggtagtttctacacagccgaggtcaagaacaacatagacttcaagaacccaatacacgagtccttctcaaaatccgggccgatgttcacacaccgtcgggttgaggagacactaacaaaagagaacctggcaatagtggagtaccagcaggtgttcaactcggccccgcgggacatgtga

SEQ ID NO: 114 - protein, 239 aa, unidentified
  REGION 1..239: note = Exemplary eGFP reporter amino acid sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 115 - protein, 236 aa, unidentified
  REGION 1..236: note = Exemplary mCherry reporter amino acid sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 116 - protein, 237 aa, unidentified
  REGION 1..237: note = Exemplary mRuby reporter amino acid sequence
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYK

SEQ ID NO: 117 - protein, 234 aa, unidentified
  REGION 1..234: note = Exemplary RRvT reporter amino acid sequence
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYNVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 118 - protein, 236 aa, unidentified
  REGION 1..236: note = Exemplary mTFP1 reporter amino acid sequence
MVSKGEETTMGVIKPDMKIKLKMEGNVNGHAFVIEGEGEGKPYDGTNTINLEVKEGAPLPFSYDILTTAFAYGNRAFTKYPDDIPNYFKQSFPEGYSWERTMTFEDKGIVKVKSDISMEEDSFIYEIHLKGENFPPNGPVMQKKTTGWDASTERMYVRDGVLKGDVKHKLLLEGGGHHRVDFKTIYRAKKAVKLPDYHFVDHRIEILNHDKDYNKVTVYESAVARNSTDGMDELYK

SEQ ID NO: 119 - protein, 231 aa, unidentified
  REGION 1..231: note = Exemplary RFP611 reporter amino acid sequence
MNSLIKENMRMMVVMEGSVNGYQFKCTGEGDGNPYMGTQTMRIKVVEGGPLPFAFDVLATSFMYGSKTFIKHTKGIPDFFKQSFPEGFTWERVTRYEDGGVITVMQDTSLEDGCLVYHAKVTGVNFPSNGAVMQKKTKGWEPNTEMLYPADGGLRGYSQMALNVDGGGYLSCSFETTYRSKKTVENFKMPGFHFVDHRLERLEESDKEMFVVQHEHAVAKFCDLPSKLGRL

SEQ ID NO: 120 - protein, 240 aa, unidentified
  REGION 1..240: note = Exemplary dTFP0.2 reporter amino acid sequence
MVSKGEETTMGVIKPDMKIKLKMEGNVNGHAFVIEGEGEGKPYDGTNTVNLEVKEGAPLPFSYDILSNAFQYGNRAFTKYPDDIANYFKQSFPEGYSWERTMTFEDKGIVKVKSDISMEEDSFIYEIRLKGKNFPPNGPVMQKKTLKWEPSTEILYVRDGVLVGDISHSLLLEGGGHYRCDFKTIYKAKKVVKLPDYHFVDHRIEILNHDKDYNKVTLYENAVARYSLLPPQAGMDELYK

SEQ ID NO: 121 - protein, 232 aa, unidentified
  REGION 1..232: note = Exemplary meffCFP reporter amino acid sequence
MALSKQSLPSDMKLIYHMDGNVNGHSFVIKGEGEGKPYEGTHTIKLQVVEGSPLPFSADILSTVFQYGNRCFTKYPPNIVDYFKNSCSGGGYKFGRSFLYEDGAVCTASGDITLSADKKSFEHKSKFLGVNFPADGPVMKKETTNWEPSCEKMTPNGMTLIGDVTGFLLKEDGKRYKCQFHTFHDAKDKSKKMPMPDFHFVQHKIERKDLPGSMQTWRLTEHAAACKTCFTE

SEQ ID NO: 122 - protein, 238 aa, unidentified
  REGION 1..238: note = Exemplary Folding Reporter GFP reporter amino acid sequence
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 123 - protein, 226 aa, unidentified
  REGION 1..226: note = Exemplary ccalOFP1 reporter amino acid sequence
MSLSKQVLPRDVKMRFHMDGCVNGHSFTIEGEGTGKPYEGKKTLKLRVTKGGPLPFAFDILSATFTYGNRCFCDYPEEMPDYFKQSLPEGYSWERTMMYEDGACSTASAHISLDKDCFIHNSTFHGVNFPANGPVMQKKAMNWEPSSELITPCDGILKGDVTMFLLQEGGHRHKCQFTTSYKAHKAVKIPPNHIIEHRLVRKEVGDAVQIQEHAVAKHFTVQIKEA

SEQ ID NO: 124 - protein, 231 aa, unidentified
  REGION 1..231: note = Exemplary tdKatushka2 reporter amino acid sequence
MSELIKENMHMKLYMEGTVNNHHFKCTSEGEGKPYEGTQTMKIKVVEGGPLPFAFDILATSFMYGSKTFINHTQGIPDFFKQSFPEGFTWERITTYEDGGVLTATQDTSLQNGCIIYNVKINGVNFPSNGSVMQKKTLGWEANTEMLYPADGGLRGHSQMALKLVGGGYLHCSFKTTYRSKKPAKNLKMPGFHFVDHRLERIKEADKETYVEQHEMAVAKYCDLPSKLGHR

SEQ ID NO: 125 - protein, 344 aa, unidentified
  REGION 1..344: note = Exemplary vsfGFP-0 reporter amino acid sequence
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGIAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVS

SEQ ID NO: 126 - protein, 219 aa, unidentified
  REGION 1..219: note = Exemplary eYGFPuv reporter amino acid sequence
MTTFKIESRIHGNLNGEKFELVGGGVGEEGRLEIEMKTKDKPLAFSPFLLTTCMGYGFYHFASFPKGIKNIYLHAATNGGYTNTRKEIYEDGGILEVNFRYTYEFNKIIGDVECIGHGFPSQSPIFKDTIVKSCPTVDLMLPMSGNIIASSYAYAFQLKDGSFYTAEVKNNIDFKNPIHESFSKSGPMFTHRRVEETLTKENLAIVEYQQVFNSAPRDM

SEQ ID NO: 127 - DNA, 1491 bp, unidentified
  misc_feature 1..1491: note = CYP76AD1 (2)
  source 1..1491: mol_type = unassigned DNA
atggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctac

SEQ ID NO: 128 - DNA, 1503 bp, unidentified
  misc_feature 1..1503: note = CycloDopa5-O-glucosyltransferase (cDOPA5GT)
  source 1..1503: mol_type = unassigned DNA
atgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtga

SEQ ID NO: 129 - DNA, 1194 bp, unidentified
  misc_feature 1..1194: note = ADHa (2)
  source 1..1194: mol_type = unassigned DNA
atgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactagg

SEQ ID NO: 130 - DNA, 825 bp, unidentified
  misc_feature 1..825: note = DODA (2)
  source 1..825: mol_type = unassigned DNA
atgaaaatgatgaatggtgaggacgccaacgatcaaatgattaaagagtcgttctttatcacgcatggaaatccaattctcacggtagaggacacacaccctctgcgtccattctttgaaacttggagagaaaagatatttagtaagaaacccaaagctattctgatcattagtgggcattgggaaaccgttaagcctacagtgaatgcagttcatatcaatgacaccattcatgattttgacgattaccccgctgccatgtatcaatttaaataccctgctcccggagcacctgagcttgccaggaaagtggaggaaattttgaaaaagtccggctttgagacggccgaaaccgatgagaaaagaggtcttgatcatggcgcgtgggtcccccttatgctcatgtaccctgaagccgatataccagtgtgccaattaagtgttcagcctcaccttgatggaacctatcactataaccttggtagagcccttgcgcccttgaaaaacgatggggtcttaattatagggagtgggagcgcaacgcaccccttggatgagactcctcattattttgatggcgttgctccttgggccgcagcttttgactcttggttacgcaaggccttgattaatgggcggttcgaagaggtgaacatatatgaaactaaagctccaaactggaaattggctcacccctttcctgagcacttttatcccctccatgtagtgctgggtgctgcgggggagaagtggaaagctgagcttatacattcctcatgggaccacggcaccttgtgccacggttcatacaaattcacaagcgct

SEQ ID NO: 131 - DNA, 1491 bp, unidentified
  misc_feature 1..1491: note = CYP76AD1 (4)
  source 1..1491: mol_type = unassigned DNA
atggaccatgcgacccttgcaatgatactagccatttggttcatcagttttcacttcattaaactactctttagccagcaaaccactaaactcctaccaccgggtcccaaaccattgccaatcataggaaacatccttgaggtgggaaagaaaccacaccggtcgttcgctaatcttgctaaaattcacgggccattaattagtcttagactcgggtcagtgactacaatagttgtgagctctgccgatgttgccaaagaaatgtttctaaagaaagatcatcctctaagtaatcgtaccatacccaacagcgttactgcgggtgatcatcacaaactaaccatgtcgtggttaccagtgtctcccaagtggaggaattttcggaaaataaccgctgtgcacttgctcagtccccaacggctcgacgcatgtcaaacatttagacatgccaaagtccaacagctatacgaatatgttcaagaatgcgctcagaagggtcaagcagtagacatcggtaaggcagctttcactacaagtcttaacctactttcaaaattatttttctcagtcgagttggctcaccataaaagtcagacatcacaagaattcaaggagctaatctggaacataatggaagatatcggtaaacccaactatgcagattattttcctatattgggctgcgtcgaccccagtggtatacggagacgattggcatgttcatttgacaaactcattgctgtttttcagtctataatttgcgaaagactagctccggatagttccacagcaactacaaccacaactgacgatgtgcttgatgtgttgctgcagttgttcaagcaaaatgagttaacaatgggtgagattaatcaccttctggtggatatattcgacgccggtactgatactacatcgtctacttttgagtgggtgatggctgagcttatcaggaacccagaaatgatggaaaaagcacaagaagagataaaacaagtgttggggaaagataaacaaatccaagagagtgatataatcaacttgccttatctgcaagcaataatcaaggagacactacgtctacaccctcccacagtctttctcttaccacggaaagccgacaccgacgtggaactgtacggatatatagttccgaaagatgctcaaattttagtcaacctttgggccattgggcgcgatcccaatgcctggcagaatgccgatatttttagtccagagagatttatcgggtgtgaaatcgatgttaaaggtagagacttcggactcttacccttcggggcaggcagaaggatatgtccaggaatgaacttagccatccgaatgctaacactgatgctagcaacccttctccaattctttaattggaagctagaaggtgacatctcgcccaaagaccttgacatggacgagaaatttggaattgctcttcaaaaaacaaaacccttaaaacttatacccattcccagatat

SEQ ID NO: 132 - DNA, 1053 bp, unidentified
  misc_feature 1..1053: note = AroGL175Q
  source 1..1053: mol_type = unassigned DNA
atgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttga

SEQ ID NO: 133 - protein, 350 aa, unidentified
  REGION 1..350: note = AroG L175Q
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHREQASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 134 - DNA, 1866 bp, unidentified
  misc_feature 1..1866: note = PvUbi2 promoter
  source 1..1866: mol_type = unassigned DNA
ggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcaga

SEQ ID NO: 135 - DNA, 1992 bp, unidentified; identical to SEQ ID NO: 26
  misc_feature 1..1992: note = ZmUbi promoter
  source 1..1992: mol_type = unassigned DNA

SEQ ID NO: 136 - DNA, 2500 bp, unidentified
  misc_feature 1..2500: note = BdUbi10 promoter
  source 1..2500: mol_type = unassigned DNA
gaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 137 - DNA, 430 bp, unidentified
  misc_feature 1..430: note = Short 35S promoter
  source 1..430: mol_type = unassigned DNA
ggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 138 - DNA, 844 bp, unidentified
  misc_feature 1..844: note = 2x 35S + TMV 5 UTR promoter
  source 1..844: mol_type = unassigned DNA
ggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataca

SEQ ID NO: 139 - DNA, 516 bp, unidentified; identical to SEQ ID NO: 42
  misc_feature 1..516: note = CsVMV promoter
  source 1..516: mol_type = unassigned DNA

SEQ ID NO: 140 - DNA, 204 bp, unidentified
  misc_feature 1..204: note = 3 UTR + 35S terminator
  source 1..204: mol_type = unassigned DNA
ctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 141 - DNA, 252 bp, unidentified
  misc_feature 1..252: note = Mas terminator (2)
  source 1..252: mol_type = unassigned DNA
ttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatc

SEQ ID NO: 142 - DNA, 714 bp, unidentified
  misc_feature 1..714: note = Ocs terminator (3)
  source 1..714: mol_type = unassigned DNA
gtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 143 - DNA, 204 bp, unidentified; identical to SEQ ID NO: 140
  misc_feature 1..204: note = 35S terminator (2)
  source 1..204: mol_type = unassigned DNA

SEQ ID NO: 144 - DNA, 736 bp, Nicotiana tabacum
  misc_feature 1..736: note = Nicotiana tabacum extensin 3 terminator
  source 1..736: mol_type = unassigned DNA
gaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctctttttttcctgaaaacaatttaacgtcgagatttatctctttttgtaatggaatcatttctacagttatgac

SEQ ID NO: 145 - DNA, 250 bp, Arabidopsis thaliana
  misc_feature 1..250: note = Arabidopsis thaliana Heat Shock Protein 18.2 terminator (AtHSP18.2)
  source 1..250: mol_type = unassigned DNA
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 146 - DNA, 263 bp, unidentified
  misc_feature 1..263: note = Nos terminator (2)
  source 1..263: mol_type = unassigned DNA
gtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 147 - DNA, 66 bp, unidentified
  misc_feature 1..66: note = Self-Cleaving 2A Peptide P2A - polynucleotide sequence 3
  source 1..66: mol_type = unassigned DNA
ggtagcggagctaccaattttagcctccttaagcaggcaggtgatgtagaagagaaccccgggcct

SEQ ID NO: 148 - DNA, 168 bp, Pisum sativum
  misc_feature 1..168: note = Pisum sativum chloroplast ribulose-1,5-bisphosphate carboxylase/oxygenase small subunit signal peptide (PeaChlSP) polynucleotide sequence
  source 1..168: mol_type = unassigned DNA
gcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgc

SEQ ID NO: 149 - protein, 22 aa, unidentified
  REGION 1..22: note = Self-Cleaving 2A Peptide P2A polypeptide sequence
GSGATNFSLLKQAGDVEENPGP

SEQ ID NO: 150 - protein, 57 aa, Pisum sativum
  REGION 1..57: note = PeaChlSP signal peptide polypeptide sequence
MASMISSSAVTTVSRASTVQSAAVAPFGGLKSMTGFPVKKVNTDITSITSNGGRVKC

SEQ ID NO: 151 - DNA, 1013 bp, unidentified
  misc_feature 1..1013: note = Tm2 MAR1
  source 1..1013: mol_type = unassigned DNA
ggagaggaagaagttcccaatcttgaaatatcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcgacgctttac

SEQ ID NO: 152 - DNA, 1001 bp, unidentified
  misc_feature 1..1001: note = Tm2 MAR2
  source 1..1001: mol_type = unassigned DNA
aggaagaagttcccaatcttgaaatatcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcga

SEQ ID NO: 153 - DNA, 724 bp, unidentified
  misc_feature 1..724: note = Pea MAR
  source 1..724: mol_type = unassigned DNA
ccatgcctcacatgttaatgtactaccaatggagggctgtacacatttatgattacgaaattttttaatatattttatagatttcttatgcatcatacaaaaatacataattattcgtaacattttggagatacatattcagatgcatcaaattctaattaaacgttaaaatattttgcagacgtatcttcgtaacaatttaaaacctatactatacatcacattcgaaggtcattttataatttaaaatattatggagatgcatcttcgtttatgtttgctcagatgaagatttaaaccttacaaacaatatgtaaaaaatgacgtacataaattcagatagtccaaaagtgtcatatataaataaagatcaataagtgtcaaaaaaagtcaagaacaacgatagagtagcataatgtcaaaataaaataaaatccatgacactactactattatatattaatgcactaatgtgtatgtctaactacatcgtctgtgcctcctctgtcagttatgtctcgtaagccatcaatcccccgtcctccggcgttgtctccggtatatcaatgtcccatgtgcctacgtcatgatggcatctaggacatgtctcacatcagacaattaggaaagatacatttgccaatgtatatctgcgcaatctccacaatgcaacgacatataggcaagacatcctcaacataatttagttgtgcatgcttctcct

SEQ ID NO: 154 - DNA, 463 bp, unidentified
  misc_feature 1..463: note = Minimal RB7 MAR
  source 1..463: mol_type = unassigned DNA
aaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 155 - DNA, 5944 bp, unidentified
  misc_feature 1..5944: note = ZmUbi - CYP76AD1 - P2A - DODA - P2A - cDOPA5GT
  source 1..5944: mol_type = unassigned DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtga

SEQ ID NO: 156 - DNA, 6452 bp, unidentified
  misc_feature 1..6452: note = BdUbi10- CYP76AD1 - P2A - DODA - P2A - cDOPA5GT
  source 1..6452: mol_type = unassigned DNA
gaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtga

SEQ ID NO: 157 - DNA, 4417 bp, unidentified
  misc_feature 1..4417: note = ZmUbi ADH PeaChISP - AroG
  source 1..4417: mol_type = unassigned DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgcagatgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttga

SEQ ID NO: 158 - DNA, 4795 bp, unidentified
  misc_feature 1..4795: note = 2x 35S + TMV 5 UTR - CYP76AD1 - P2A - DODA - P2A - cDOPA5GT
  source 1..4795: mol_type = unassigned DNA
ggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtga

SEQ ID NO: 159 - DNA, 2941 bp, unidentified
  misc_feature 1..2941: note = CsVMV ADH PeaChISP - AroG
  source 1..2941: mol_type = unassigned DNA
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgcagatgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttga

SEQ ID NO: 160 - DNA, 9815 bp, unidentified
  misc_feature 1..9815: note = PL-888 construct
  source 1..9815: mol_type = unassigned DNA
ggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtagaacaagacgggctgcatgctggatcacctgcggcctgggtcgaaaggctattcggttatgactgggcgcagcaaaccattgggtgcagcgatgcagccgtctttcgactcagcgcccaaggtagacccgtattgtttgtgaaaactgacttatccggggcactgaatgaattacaagacgaagcagccagacttagttggttagcgaccacaggtgtgccctgtgcagcggtccttgacgtagttaccgaggcaggacgggactggttattgcttggagaagtaccgggccaagacctgttatctagccacctcgccccggctgaaaaagtctcaattatggcggatgctatgaggcgacttcatacattggacccagccacgtgtccctttgatcatcaggcgaaacacagaatagaacgtgcaaggacacgaatggaggctgggctcgttgatcaagacgatttagatgaagagcatcaaggcttggcacctgctgagttatttgctcggttaaaagctagaatgccagacggggaagatcttgtcgtaacccatggcgatgcctgcttgccaaatattatggtggagaatggtcgtttctcagggtttatcgattgtgggcgacttggcgtggccgacaggtatcaagatatagccctagcaactagagacatagccgaagagttgggtggggagtgggctgacagattcctcgtgctatacgggattgccgctcccgattcccagagaatcgcattttatcgactcttagacgaattcttttaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggagctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcggtagtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 161 - DNA, 16718 bp, unidentified
  misc_feature 1..16718: note = PL-1044 construct
  source 1..16718: mol_type = unassigned DNA
ggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtagaacaagacgggctgcatgctggatcacctgcggcctgggtcgaaaggctattcggttatgactgggcgcagcaaaccattgggtgcagcgatgcagccgtctttcgactcagcgcccaaggtagacccgtattgtttgtgaaaactgacttatccggggcactgaatgaattacaagacgaagcagccagacttagttggttagcgaccacaggtgtgccctgtgcagcggtccttgacgtagttaccgaggcaggacgggactggttattgcttggagaagtaccgggccaagacctgttatctagccacctcgccccggctgaaaaagtctcaattatggcggatgctatgaggcgacttcatacattggacccagccacgtgtccctttgatcatcaggcgaaacacagaatagaacgtgcaaggacacgaatggaggctgggctcgttgatcaagacgatttagatgaagagcatcaaggcttggcacctgctgagttatttgctcggttaaaagctagaatgccagacggggaagatcttgtcgtaacccatggcgatgcctgcttgccaaatattatggtggagaatggtcgtttctcagggtttatcgattgtgggcgacttggcgtggccgacaggtatcaagatatagccctagcaactagagacatagccgaagagttgggtggggagtgggctgacagattcctcgtgctatacgggattgccgctcccgattcccagagaatcgcattttatcgactcttagacgaattcttttaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatggtagtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctactagaattcgagctcggagaggaagaagttcccaatcttgaaatatcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcgacgctttacgaattcccatggggagctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgcagatgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttgatgagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcggtagtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 162 - DNA, 17426 bp, unidentified
  misc_feature 1..17426: note = PL-1233 construct
  source 1..17426: mol_type = unassigned DNA
gtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaatgacaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactaccactctgtgggtctcatgccgaattcggatccggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggagccatgcctcacatgttaatgtactaccaatggagggctgtacacatttatgattacgaaattttttaatatattttatagatttcttatgcatcatacaaaaatacataattattcgtaacattttggagatacatattcagatgcatcaaattctaattaaacgttaaaatattttgcagacgtatcttcgtaacaatttaaaacctatactatacatcacattcgaaggtcattttataatttaaaatattatggagatgcatcttcgtttatgtttgctcagatgaagatttaaaccttacaaacaatatgtaaaaaatgacgtacataaattcagatagtccaaaagtgtcatatataaataaagatcaataagtgtcaaaaaaagtcaagaacaacgatagagtagcataatgtcaaaataaaataaaatccatgacactactactattatatattaatgcactaatgtgtatgtctaactacatcgtctgtgcctcctctgtcagttatgtctcgtaagccatcaatcccccgtcctccggcgttgtctccggtatatcaatgtcccatgtgcctacgtcatgatggcatctaggacatgtctcacatcagacaattaggaaagatacatttgccaatgtatatctgcgcaatctccacaatgcaacgacatataggcaagacatcctcaacataatttagttgtgcatgcttctcctcgctactagaattcgagctcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctctttttttcctgaaaacaatttaacgtcgagatttatctctttttgtaatggaatcatttctacagttatgacggtaatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagcgctttacgaattcccatggggagaggaagaagttcccaatcttgaaatatcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcgacgctcagagaattcgcatgcggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgcagatgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttgatgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 163 - DNA, 17935 bp, unidentified
  misc_feature 1..17935: note = MAR - PL-1233 construct - MAR
  source 1..17935: mol_type = unassigned DNA
gtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaatgacaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactaccactctgtgggtctcatgccgaattcggatccggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggagccatgcctcacatgttaatgtactaccaatggagggctgtacacatttatgattacgaaattttttaatatattttatagatttcttatgcatcatacaaaaatacataattattcgtaacattttggagatacatattcagatgcatcaaattctaattaaacgttaaaatattttgcagacgtatcttcgtaacaatttaaaacctatactatacatcacattcgaaggtcattttataatttaaaatattatggagatgcatcttcgtttatgtttgctcagatgaagatttaaaccttacaaacaatatgtaaaaaatgacgtacataaattcagatagtccaaaagtgtcatatataaataaagatcaataagtgtcaaaaaaagtcaagaacaacgatagagtagcataatgtcaaaataaaataaaatccatgacactactactattatatattaatgcactaatgtgtatgtctaactacatcgtctgtgcctcctctgtcagttatgtctcgtaagccatcaatcccccgtcctccggcgttgtctccggtatatcaatgtcccatgtgcctacgtcatgatggcatctaggacatgtctcacatcagacaattaggaaagatacatttgccaatgtatatctgcgcaatctccacaatgcaacgacatataggcaagacatcctcaacataatttagttgtgcatgcttctcctcgctactagaattcgagctcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggtagcggagctaccaattttagcctccttaagcaggcaggtgatgtagaagagaaccccgggcctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggatccggagcaaccaactttagcctgctcaagcaagcaggagatgttgaggaaaatcctggccccatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaatgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctctttttttcctgaaaacaatttaacgtcgagatttatctctttttgtaatggaatcatttctacagttatgacggtaatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagcgctttacgaattcccatggggagaggaagaagttcccaatcttgaaatatcgaaggttcgcctttctgttgtaatattgctcaatcctctacttttgtgccgccatctgtatcaaagttgcttcccttatctcctttgtcaaatctaaatttgttaaaagttctttctcatttgttacttcatttgtatgtacgtatcttgttcttggctctcctatttccaccgaaattaaggcctcagtaccataaataagtgagaatggtgtttctcttgtgcttatcttcgtcgttgtcctgtaatcccatactagcccaagtagtacttcagggaacctgccttttgatgattctaaccgtttctttatattgttgacaataattgtatttgtcgattatggtttcccattagctatgggatggcagtgcgccgaaataatttgtttaatttcccagattttgagaaaatcggtaacttttgaacctacgaattgtggcccattatcgcgtactatttttcttgggacacaaaatcgacatattatgttcctccaaataatatcaacaacctctttctctcgatctatttaaaggcacatgcttctacccacatagaaaaataatcagttaagattaataaaagtcgttcctttcctggtgctttaggtagaggacctacaatacataacccacttcataaacggccacggtgctatgattgaatgaagaaacactgctggttgatgcatattgtttgcaccccgttgacatttatcacaccttctaataaaaattttcgctttgtgttctattttttgcaaataataccgccccttttagcgtctttaaaatgaccttcctccaacgtgattgcaacaatgttctttatgtacttcttgcatcacatattcagtttgtgatagaccaaggcaccgtgctaagggtcccatgaacatttcgatacaaattttcttctattaagcaatattgagcagctttcactcgcaataattgggatttttaatcgacgctcagagaattcgcatgcggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgcagatgaattaccagaacgatgacttaagaatcaaagaaattaaagagttgttaccacctgtggctcttttggaaaaattcccagcaactgagaatgctgcaaacacagttgctcatgcaagaaaggctattcacaaaatcttgaagggtaatgatgacaggttacttgttgtgatcggaccatgctcaatacatgatcctgttgctgcaaaggaatacgctactagattgcttgcattgagggaagagttaaaggatgaacttgagattgttatgagagtgtacttcgagaaaccaaggaccactgttggttggaagggacttatcaatgatcctcacatggacaactccttccaaattaatgatggtttgagaatcgctaggaaacttttgcttgatattaacgactcaggtttgccagctgcaggagaatttttagatatgatcacacctcagtacttagctgaccttatgtcatggggtgctataggagcaagaacaaccgaaagtcaagttcatagggagcaggcttccggtttgtcttgtccagtgggattcaaaaatggtactgatggaacaattaaggttgctatagacgcaattaacgctgcaggtgctcctcattgttttctttctgttacaaaatggggacactcagcaatcgtgaataccagtggtaacggagattgccatattatcttgagaggtggaaaagaaccaaattattcagctaagcacgttgcagaagtgaaagagggtttgaacaaggctggattacctgcacaagttatgatcgatttctctcatgctaactcctctaagcaattcaagaaacagatggatgtttgtgctgacgtgtgccaacagatcgctggtggagaaaaggctattattggtgttatggtggaaagtcacttagttgagggaaatcaatcattagaaagtggagagcctcttgcttacggaaaatctattaccgatgcatgcatcggttgggaagatactgacgctcttttgagacagttggctaacgcagttaaggcaagaaggggttgatgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgcttgtgtgagaccgaggatgcacatgtgaccgagggacacaggtaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 164 - DNA, 839 bp, Cauliflower mosaic virus
  misc_feature 1..839: note = Exemplary Cauliflower Mosaic virus promoter (2x CaMV35S) (2)
  source 1..839: mol_type = unassigned DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatac

SEQ ID NO: 165 - DNA, 276 bp, unidentified
  misc_feature 1..276: note = Exemplary Agrobacterium tumefaciens Nopaline synthase gene promoter (NOS) (2)
  source 1..276: mol_type = unassigned DNA
agcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctca

SEQ ID NO: 166 - DNA, 792 bp, Arabidopsis thaliana
  misc_feature 1..792: note = Exemplary Arabidopsis thaliana Actin 2 promoter (AthAct2) (2)
  source 1..792: mol_type = unassigned DNA
aggagtcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaa

SEQ ID NO: 167 - DNA, 1300 bp, unidentified
  misc_feature 1..1300: note = Exemplary Epipremnum aureum ribulose bisphosphate carboxylase/oxygenase activase 2 promoter (rrEaCons1) (2)
  source 1..1300: mol_type = unassigned DNA
cctcaaccttcgctcacagtgaaggcttgaaactcgctttttaacattgtaagtgggctgattttgaactcatctcatcgtaaatctttaagctttgacttcccacgatgttgtccagtctattagattttttatggtttttttttcttttttcgctgaaagttcctacttaaaatagtcacccactaggtacagaagagtcagctacatgaaaaataccttaatatagaaaaacgtatttattgtattaaaatttgaaccctccccacttaaaatgatgcgtaccacttagacctagttgagatttattgttgcacctgggagagagttgaatagggtccggattcccacttagtttctctggaatctagatagggcggtcagctttatcttaattagtgacaaggcactagttggagttagtttttatattgaacatactcttaaacttttagttccctattttgagagaaagtatttgaagtaattttaaacttttggttaaatcttccacttttgaccaaaagttcaaaattaaagtttcccaagttcaagaaagaatggtatcattagcccatataagaactaaattaaaatcagtttgattcattcttattaagctccaacatactcaacagcacaaccaacagcatgacttgtgtaaactgaaaaactcagagagagagagatagagactctgaacgagtggtgctgagcagcagtggctgcttcatgaagagtttggcgtgacgacaaaaccatcaaaaacacagaagaggaatttcattgccgacaatcaccatgtctctgtaatactgctggtcctgatgaaatgcttgaaggaaaaaaaactggcattaaagaggaggggaaaaaaccgaaaattttagtggagtcgggaagcccgggaacccgaaccattcctggcgtctgacgtcctccgctgccgagaggatgctgtagctgatgggccccacttccccacactccccaacttccaacgtcaggacacgactctatctgcgcagaagcaaccaaccctgatgcgccacgtgtcgccccaccccaatccgcagtgtgtggccgttgtggccctcgcgatccaatccacaggatgcttcactctcctcctctcctccgcaagccaaacgggaaaataacggagcagggcagactccagagcctccgcaggccgctttatatataactcgccctcccacgcctcctacggtcatcactgccgcgaggagctttgcttttggtggacgcggcgatctccccccatctccttctcggtctt

SEQ ID NO: 168 - DNA, 929 bp, unidentified
  misc_feature 1..929: note = Exemplary Epipremnum aureum abscisic stress-ripening protein 2-like promoter (rrEaCons3) (2)
  source 1..929: mol_type = unassigned DNA
gttccactcgaggcaggaaaaatctctggatttggacacttaaccgacccccattaacaccccacctcacatcagagcacggtttgcccactcaacttgtcaggcaaaccacatcttatctcaaaagctatgagttacaacgtcagataactaatttaaataataatataaatttaaaatataaattatattttttattaaattaaaagaataatattttttaaatatctaattttatccaatcaaattcaagttcaactgatctatattaaataaaaaaattaatacgaatccaaattttaagttgacaaataaatgaattttgaataaaagaatcacaaataaaaaattacgttttcttggcgtatatcaccatgcttgtgttcgtttaagagatttaagcaatcatggacgtctgcttatccacggatgtgaaatattaaatgataaaatactatattatcttatattatagaaaaataaattttaaatgagaagtgggtatttattatgttttcattcaacatacgtgcgaaagttttatctagatagattagcgttagcatcactcaagaattttttttattttcttaactgcttcaaaaaaagaaatataaagggattggcccacgttaattagctagaaaaagtgggattgaaacgggtgttatccacttcacattctgtgagcgaatccgatgcgtgaagccccgccatcctgacccgaccgctgttcccccctacccacgaagaagccgtctgtccgtctcttcaatctctatacttccccttcgcctgctgcgtacactcccgtggctataaataaccaccacagcctctctgatttcttcgtacccattactgcaacacctctacagctactagccgtgtcgcccgcccccccttaaggtcattctaccactgccagt

SEQ ID NO: 169 - DNA, 1114 bp, unidentified
  misc_feature 1..1114: note = Exemplary Epipremnum aureum carbonic anhydrase 2-like isoform X1 promoter (rrEaLeaf4) V2
  source 1..1114: mol_type = unassigned DNA
cgcacgtagccttcgttactcatcttgttgttcgtctaatttggagagatggtttcaagcatttgacaatccaaggagacaaagtcattagtattaatgtttctctgttaattaattgtctccctgatatcctgtctcaagtatgtttatgtgtgtgtgtgtgtgtaaatataaatataaagaacaatatgtgataaaggataaccattctgcatggtggatttgtgttcattaattaatatagttctttctttccatcatttgatttcatttcatacactagtactttggtaccatgtttatttttcaaggtttatcgaacaggaattattcagaagatataccaaaaatcgattggattcattctctattcagactgttaattgttaaccatcgatttaaacatgtcatcttaagggaaattaagaaactagattgtgtttacgttttccacactgttagaccttctatagtatcttcattgttctcgagtcgattggtagtattggaacgaactagcatgcatgtgtggaacaccccctcttatatactgcaaaaaatgaaaaagaaaagaaaatggaccatcactttgattttttagggtttggtggcttcaagacacgatgcttggctgggtgcaattaaactgtgccataaaaatgtactatgctattcaataatcgatttcatgagacatggtacatgtcatatttcataaatgacgtggtacatgccaaatttcataagttttcttgtctagaaacttaataaattactattcgcatagaaatcctgaatttttactatttctgatttcccccacccccagaattttaaggttgaagctatcagaaaaacaagaattattatatataatccatctgcaatgcatgagattagcgatacacctgcaacgccatcacctattccatccaacgattacatgacactgtcatctccaagccttctctctctctctctctctccctctcccttatttgaagcagaagccatggttgatccggctttcgctttccttatcctaacccacccccgtcgcagagactatatatcgagccctccacccctcctgggacgggtgtgaaagagagca

SEQ ID NO: 170 - DNA, 1079 bp, unidentified
  misc_feature 1..1079: note = Exemplary Epipremnum aureum dehydrin COR410-like promoter (rrEaStem3) (2)
  source 1..1079: mol_type = unassigned DNA
ctgaggacgcttcgagatccactgaccatgccactttttttttacgtgaacgaggcaagtcggcattgacgagcggggatgaaaagggccgtggagcgaaggggacacgcacgctcataatactgttctgtacggcttatatagtataaacagatccagcgcagcgcccgcgcatgtggcggggtattgggggaggcgatggcgcgcgtctgctcccccgccgtgaggccaaggacctccggtaggggcgcaccgctcgcggtgtatggcggccgtaccgtggacatgcatgtatggtgggctttttttaagtttgccccggataagtgttactgttgtggacatgcacatgcatacgatgatggggtccgtctgggtccgttgctctactcatccgatgccacgcaagctctgtagtaaatgtatgtatatattcgtgtgagaaagaggaacgaaaagggacaactaagcgaagtccgatggctcatcttaatgattaaattacaaaaaaaaattatttagatatcttcgtatcaagtctctagagaataatctgtcatttaaagtttgaggttattttatggatatttctttctcctttaatgacttataaatattagattttacttctctcagttataaaatcactcatcattccaactgagttatttatctaagatttgatgacaaggggaacgacgattacgatgggcgctctccaagcgttgctgtggaatttctcgcggtgagtggcgatgacacgtgaaactttgtcacaactactccaagaatcccactagccattagcttgtatgatattaatactgagactggttattaacaaacatctaacaccaccttttatttaccagacgaggacggtaacggaaaacaggggaatgaaagcaagagaaagccgacatcggaccgacgttcctcgaggcccgatctgatccactccaacccgccatcgtcagcatcaccgtctcaaatcaagtccatttatcgcccgctgcgaaagggaaaggcaaagggtttgaaaaaaaaaaagaaaggcaacgaaagggggacgaaggtgg

SEQ ID NO: 171 - DNA, 1861 bp, unidentified; identical to SEQ ID NO: 28
  misc_feature 1..1861: note = Exemplary Promoter PVUBI2 (2)
  source 1..1861: mol_type = unassigned DNA

SEQ ID NO: 172 - DNA, 1861 bp, unidentified; identical to SEQ ID NO: 28
  misc_feature 1..1861: note = PvUbi2 promoter (2)
  source 1..1861: mol_type = unassigned DNA

SEQ ID NO: 173 - DNA, 426 bp, unidentified
  misc_feature 1..426: note = Short 35S promoter (2)
  source 1..426: mol_type = unassigned DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 174 - DNA, 843 bp, unidentified
  misc_feature 1..843: note = 2x 35S + TMV 5 UTR promoter (2)
  source 1..843: mol_type = unassigned DNA
ggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatac

SEQ ID NO: 175 - DNA, 276 bp, Solanum lycopersicum
  misc_feature 1..276: note = Solanum lycopersicum Histone H4 terminator (TerSlHisH4) (2)
  source 1..276: mol_type = unassigned DNA
ttatgttggtgatatggtggtaaatgtagggatttagtttacaattgcgtatgtctgtgttggatatctgtagtgctgttcttatggcttagatcttgtaatttctcattacagtatcaatgaatagatatcagtttctagtgatgacattggttcgtcttttagctgttgattaatttttcttaattgattcatcctattgcaattcttctgaatttaaattgtatactgtgaaattaagaaaattcttgaaattaatgagaatttgagtaatag

SEQ ID NO: 176 - DNA, 263 bp, unidentified; identical to SEQ ID NO: 146
  misc_feature 1..263: note = Agrobacterium tumefaciens nopaline synthase terminator (TerNos) (2)
  source 1..263: mol_type = unassigned DNA

SEQ ID NO: 177 - DNA, 250 bp, unidentified
  misc_feature 1..250: note = Agrobacterium tumefaciens mannopine synthase terminator (TerMas) (2)
  source 1..250: mol_type = unassigned DNA
ggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatc

SEQ ID NO: 178 - DNA, 714 bp, unidentified; identical to SEQ ID NO: 142
  misc_feature 1..714: note = Terminator OCS (2)
  source 1..714: mol_type = unassigned DNA

SEQ ID NO: 179 - DNA, 278 bp, Solanum lycopersicum
  misc_feature 1..278: note = Solanum lycopersicum Rubisco small subunit 3C terminator (Ter SlRbcS3C)
  source 1..278: mol_type = unassigned DNA
atatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttccccattcaagttacttatgaaatagaaatcctcttctccgactactttatttgtccgaaagtcttgtggctgctatataa

SEQ ID NO: 180 - DNA, 216 bp, unidentified
  misc_feature 1..216: note = Agrobacterium tumefaciens gene 7 terminator (Ter Atug7)
  source 1..216: mol_type = unassigned DNA
gatcccccgtcgacagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagat

SEQ ID NO: 181 - DNA, 667 bp, unidentified
  misc_feature 1..667: note = Epipremnum aureum rrEaH3Blast2 terminator (Ter 7.1)
  source 1..667: mol_type = unassigned DNA
gctgtaaagaaattgatgggcagtgggcttttgttactagttagtaggagaggttgcttcagtttcgtccgtacctgttcttgaccttctgtttctggagtctgtactccgtttgttgtaaagtcttgtcctttttttaaaacttctttctatccactgttgaatgagccagtagatgctgtcctgttacgcgtttctcttctcttgcacatgcacagtctccgttttgtaggatgctgaacgaagctctcgggtttatggaggtcaatccctaagtattgtcgattcaaaagggtgatgtttttttcccccaacaaagctcttcagtgagttcaaccaagtgggtgagatgtgtataggttactggacaatcttgttggtttggagaggagaaaaagtagctatattgatctgtgccagtgctagcacagggagagtcttatctttttgggttagtgttacagctagatgattgagatgatcatctgcacttgatttgatcagctggttttgtctttgtaagattagcctgtcacttgacgaaaaaaagcggtttgtctgtcctcggttacgattcagactggtttggatgacgtccatattaagatcctgtatttacgtttgctgctctcattttctgcaagctttccgaggatgtccaaaagct

SEQ ID NO: 182 - DNA, 310 bp, Solanum tuberosum
  misc_feature 1..310: note = Solanum tuberosum Proteinase inhibitor 2 terminator (Ter StPinII )
  source 1..310: mol_type = unassigned DNA
ctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 183 - DNA, 471 bp, Pisum sativum
  misc_feature 1..471: note = Pisum sativum Rubisco small subunit 3A terminator (Ter Pea3A)
  source 1..471: mol_type = unassigned DNA
tccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 184 - DNA, 252 bp, Arabidopsis thaliana
  misc_feature 1..252: note = Arabidopsis thaliana Heat Shock Protein terminator (Ter AtHSP)
  source 1..252: mol_type = unassigned DNA
tctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaatt

SEQ ID NO: 185-186 - skipped (2 entries)

SEQ ID NO: 187 - DNA, 165 bp, Arabidopsis thaliana
  misc_feature 1..165: note = A.thaliana ribulose-1,5-biphosphate carboxylase small subunit (rbcS) Choroplastic signal peptide
  source 1..165: mol_type = unassigned DNA
atggcttcctctatgctctcctctgccgctgtggttacctccccggctcaagccaccatggtcgctccattcactggtttgaagtcatccgcttctttcccggtcacccgcaaggccaacaacgacattacttccatcacaagcaatgggggaagagttagctgc

SEQ ID NO: 188 - protein, 55 aa, Arabidopsis thaliana
  REGION 1..55: note = A.thaliana ribulose-1,5-biphosphate carboxylase small subunit (rbcS) Choroplastic signal peptide
MASSMLSSAAVVTSPAQATMVAPFTGLKSSASFPVTRKANNDITSITSNGGRVSC

SEQ ID NO: 189 - DNA, 24191 bp, unidentified
  misc_feature 1..24191: note = PL-418 construct
  source 1..24191: mol_type = unassigned DNA
ttcgtgtccctcggtcacatgtgcatcctcggtctcagctcagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccctcgaggaattccacaagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagcctacctagtcccattgacaactacaagatcgtgctttttctcagccatgtcagcggagggcactatttgaggtgggttaattgtgggagagggtagcatcaggggcttatcgaaggtgacctccgcctgatcattgttcccttccagcattagttgtttcctcaccagatcatgcaaataaccgcttagctgctttttcaccatctcaaaagcccagtccattctatccaactgctccatagcattcttgttataaaggaataggccgtaaaacaattcaaagctgtcgcgtgctgtgttatcaactagatttagtaatgattcgtatccctttgtgttaattggcgtattttcaagtgctagtttttccagtacccttcccatcatgtgtgtaataaattgtgagcctgcggcatgtttgtcatgatctacgcatgacatttccaccattctacacccggcgtttctaaaaacatctaaaaatgcctcagctcttgacgtccgagattggtcgctgcccacccttactttgtcaaatacgaatgggagtccgccccacccgtctttcccgctgtctggcccaaacatagggtgggtacagagtatatcaaaatctttgggcaagagttgcagaaataaagagcgagggaactctttcacactcaaaacatcagcgaacagggttgatctgcgcaatctatggagaggtaatgaccgcaacaccttctcagtagacagtatagatgtacacaaaagaattacttcaggatgttcctcacacaagtcatctgcatcagtaaaatattcaactccaatctcttttgccgcccgggaatagtctgatcgagaatatgctagtactctatgaccttgttttgccatagtctttgcgagaaattgcccaaagtttccgaaccctatgatagcaattttgagttttacatcccttttagatacatccaagtgatcgtttgaaaccctagttgcggctgaatctctgttgaatacggactccgatgcagagccaccccctccacacctaaccacaagatgttgacgagggtgtcgtagcggcaaggatagatgcgaaggtggtgaagagaatgcggggcattgctgtggagggtgtgtggttgcggcagctgcggtagcggtagcagttgtgcttgatggatgaaaagaggatagtgatatcattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagaggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctccgcatgcgaattctctgagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcaagcatcttctttagatttctttcccctacaagaaactataaactctacaaaattatccattgccttaacagaagatcctttgtaatctccctcaattttccaagcatccttcaatttctccccaatctcaacagctttcttcttcatttcccacccacaactcccctcctttctatccaacaccatttccaccacactcttcacccactctttcttcacctccccttccaaccccctcgtcaactcgaccgccacccccatttcctccaccatcatctttgaattatacgcttgctcggccgccaacggccacccgagtattggcactccttctcttaaaccttccaataccgaattccaaccacaatgactcaaaaatccaccagtagcctcatgcctcaaaatatccaactgaggcacccaatctctcaccaacatcccctgttttttctccatcattctttcctcaaacttctcaggcagccattcgggtttaaactccccattgaaatcgaacccgaatggcggccgaatcacccacaagaacgggatcttactctcctccaaacctttcgctaactccatcatctgaatcggagtcactgtattctgtgacccgaaacaaatgtacaaaactgagtcattttcaaattgattcaaccacctcaaacaagcctcttccttctcatcatcatcttttttcgaagatgtcataatcaaaggaccaattccccaaataggagatttcgtgaaattcctaaaaacttgaaaccctaacggttcaatctcctcgatggaattacacaaccacccagaacaattcaaggatgatttaatctgaggttggaaaaacctcgaccattcatcggacccatcagcaaaccttaaaaaccgatgaagctgattacgtcggagtttccgatcttccgggaaaccagccaatgaaaactcctcatcatcgccgacattccggtgaggtagattgctccagattgaaacatacgcaccgacaccgtaggccccaccagtgttgaagcagattccggtggacccaactgatcgggccacgttatcggcccagcccaagaacacgtcgaagattatgcacacgggtgggtcgtgcaggtggtggcgcgtgaggtagtcacgtacatgaggttcgagtgaagtggatgcatggaagagtttgatgatggaggttaatgagagtttatcggtattttcggtgttgggtgggacaccgtggtcggtgctagtgaaggggagttcgatgatgttaaggttgatgttgaagttgtcggattgacggcggagattagcggcgtttaaaggggtagtgaggagagagagattgaagggtgttagagagagaaggcggtgagcgaggtggaggaatggtcggagatggccttgtgctaaaaatgggaggaggattatgtgttgttgttcttgttcttttgttgatgactccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccccatgggaattcgtaaagcggataatttatttgaaaattcataagaaaagcaaacgttacatgaattgatgaaacaatacaaagacagataaagccacgcacatttaggatattggccgagattactgaatattgagtaagatcacggaatttctgacaggagcatgtctacaattcagcccaaatggcagttgaaatactcaaaccgccccatatgcaggagcggatcattcattgtttgtttggttgcctttgccaacatgggagtccaagcttaggtggaagtaaacttgtaggagccatgacataaggtaccatggtcccaactagtatgaataagctcagccttccacttttcaccagccgccccaaccacgacatgcaatggataaaaatgctccgggaatggatgtgccaattcccagtttggtgctttggtttcatatgtattcacttcttcaaatcttcctttagtgagagctacgtcaagccatgagtcaaagtcagcagcccaaggagcaacaccaccatgataatgaggggtttcgtccaaagggtgtgttgcacttccggaaccaataatgagtacaccttcgtctcttagaggcgctaatgctcgtcccaagttgtagtggtacgttccgtccaacttcggctggacggagagctgacataccggtatgtcagcctccggatacatgtacataagaggcacccatgcaccatgatcaagcccacgttttttgtcaatgtgcactgtgtcgaacccggattttttcataatttcttctactctgctagccaagtctggtgatcctggagctggatacttcaacttgtacatggcttgaggatagtcatcgaaatcgtagatagtatcattgatatgaacagcattaacagcaggatgatcagtttcccagtgaccagagatcacaagtatcgccttcggcttcatcgtaaagattttttctctccatgtttcaaagaatggccttaagggatgtgtatcttctacggtcaaaatcgggtttccatgggttatgaagaaagtttctttgatcatgctattatcttctccactcattttcattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagaggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctccgagctcgaattctagtagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcaatacctaagaacgggaataatctgaagaggtttagtcttttgtaaggcaatcccaaatttctcatccatgtctagatcttctgctttcataccttcttcaagcttccaattgaagaattgaagaagcgtagctaacatcaaagttaacattctaatagccaaattcatcccggggcatattctccttccagctccaaaaggtaagagtccaaaatctcttcctttcacatcaatttcggatcctaaaaacctctcaggtaaaaacacgtcaggtttcacccacgcttgagggtcccttccaatggcccataaattaacaagtatttgtgcatctttcggcacaacatagccatataattcaacatcactatcagctttacgaggcaagagaaatacagttggtgggtgtagccgcaatgtttctttgataatggcttgtaagtaaggtagcttaataatgtctgattcttgaatttgtctatccttgcccaagacttgctcaatttcttcttgagctttttccatcatcttaggatttcgaattaactctgccattacccattcaaaagtacttgatgtagtgtcagtcccagcatcaaaaatatccacgagcagatgatttatctcgcccatactgagctctttttgtttgtggaggtcaagaagaatgtctagcacatcatcggtcgtcttagtcgaagcagaatcctggccgttgccaatcctttgacatattatgctttgaaaaactgcaatcaacttgtcaaaattagacgccaatcgacgtcgaattcctgatgggtctaagcatcctaagattggaaaataatcagcgtagttaggtttgccaatatcttccataatattccatataagttctttgaattgttgagatgtatgggatttatggtgggctaattcgaccgaaaagaataattttgataacaaattaagggaggttgtaaatgctgccttgccaatatcaacagcttgtcctttttgtgcacattcttgtacatattggaaaagctgttgcactttggcatgtcttagggtttggcaagcatcaagtctttgaggagaaagtaaatgaacggcggtgatttttctaaagttccgccactttggagaaacaggcaaccacgacatagtcagcttatggtgatccccagcagtgacagaattaggaacagtgcggttagaaagtgggtggtctttttttaagaacatttctttggctacttcagccgaggatacaacaatagttgtgacacttcctagacgtaatgatatcaatggaccatgaatcttagcaagattagcaaatgactggtgaggtttctcaccaacttcaaggatgtttccgattagtggcaatggtttaggaccggggggaagaagtttggtagtttggctagtgaataacattttaatgaaatgaaaaacaacaaaccaaatagcaagtatcattgctagtgttgcatgatccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccaagcttgaattcttgcggagtcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcgctctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgataaatgctttcactacgtcaatctataagatttttcaagccagccacaagaactttgtgtagctctagatatctgcttcagcaaaaacccgtgaccgtgttcgggattttgaatttgaccgaggacagtttctttgatgaatcgagacgattggacccagcaggggctgttacagctgcgattgagatgctgagggtcggctccgatgtagtcgacgttggacccgctgcctcccacccagacgcacgtccagtctctccagctgacgagatacgtcggatcgcccctcttttagacgcccttagcgaccagatgcatagagtgtcaattgatagctttcagcccgaaacacagcgctacgccttaaaacgcggcgttggctatcttaacgatatacagggttttccggacccggctctctatccggacatagcagaagcagattgtagactagttgtcatgcatagcgctcaacgcgatggaatagctacaagaacaggtcatttaagaccagaagatgctctcgatgaaatagttagattctttgaagcaagggtcagtgctttgaggagaagcggggtcgcagccgatcgcttgatattagatcccggcatggggttctttctaagtcctgcacccgaaacttctcttcatgttctcagcaacctccaaaaactaaagtctgccttggggcttccacttctagtaagtgtatcacgcaaaagtttccttggggcgactgttggccttccagtgaaagatctcgggccagcttcactggctgcagagctgcacgcgataggtaacggcgctgactatgtcagaacccatgctccgggcgatctgagatccgcaattacaatctcagaaacccttgcaaagtttagaagccgtgatgctagagataggggccttgatcatgcataggcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctggatccgaattcggcatgagacccacagagtgttcaaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcaaccatgactactgtccggtccaacagatccgagcaggggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcaagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggatctgccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtaatggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcgctgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcctcgggaattggccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcggctggcacatacaaatggacgaacggataaaccttttcacgcccttttaaatatccgattattctaataaacgctcttttctcttaggtttacccgccaatatatcctgtcaaacactgatagtttaaacggatcac

SEQ ID NO: 190 - DNA, 12973 bp, unidentified
  misc_feature 1..12973: note = PL-450 construct
  source 1..12973: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgactaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagggaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgactgcaatcaaaatgaatacgaatggtgagggtgaaacccaacatatacttatgataccatttatggcccaagggcaccttcgaccgtttctggaattggccatgttcttatacaaacgatcacatgtcatcataactctgctaacaactccccttaacgctgggttcctgagacatctgttgcatcaccattcctacagctcctctgggatacggatcgtagaattgccgtttaattcgactaatcacgggctcccacccggtatagaaaatacagacaaattaacactgcctttagtcgtatccttgtttcattctacaatttccctagacccccacctacgcgattatatttcgagacacttcagtcctgcacgacccccactgtgtgttattcacgatgtttttctcggttgggtggaccaagtagctaaggatgtagggtccacaggcgtcgtttttacgaccggcggggcttatggcacctctgcatatgtatctatatggaatgacttaccacaccaaaattactccgacgatcaggagtttccattgccaggattccccgagaatcataaatttcgaagatctcaactgcatcgtttccttaggtatgctgatgggtctgatgactggtctaaatatttccaaccccaactacggcaaagtatgaaaagttttgggtggctatgcaatagtgtggaagagattgaaacattgggcttctcgattctaagaaactacaccaagcttccaatctggggcataggcccacttatagcctccccggtacagcacagttcaagtgacaataactctactggagcagagtttgtgcagtggttgagtcttaaagaacccgacagtgtattatatatatctttcgggagtcaaaatactatttcaccaactcagatgatggaattggctgcagggttggagtcgagtgagaaaccgtttctgtgggttatacgtgccccatttgggtttgatattaatgaggaaatgcgtcccgaatggcttcccgaggggtttgaagagaggatgaaagtcaaaaagcaagggaagcttgtttacaaattggggcctcaattggaaatccttaatcacgaaagtatcgggggttttctaacgcattgcgggtggaactctattcttgagtccctaagagaaggtgttcccatgttgggatggccactagcagctgaacaagcttacaacctaaaatatctagaggatgaaatgggtgtagcagtcgaactagccagaggcctcgagggggaaatttccaaagaaaaggttaagagaatagttgagatgatcttagagcggaatgaaggttcaaaaggatgggagatgaaaaacagggcagtggaaatggggaaaaagttaaaagatgccgtcaacgaggaaaaagagttgaaaggttcttcagtcaaagcaatagacgatttccttgacgctgtgatgcaagcaaagctcgaaccgtcattgcaatgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 191 - DNA, 16237 bp, unidentified
  misc_feature 1..16237: note = PL-451 construct
  source 1..16237: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgactaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagggaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggagtcatcaacaaaagaacaagaacaacaacacataatcctcctcccatttttagcacaaggccatctccgaccattcctccacctcgctcaccgccttctctctctaacacccttcaatctctctctcctcactacccctttaaacgccgctaatctccgccgtcaatccgacaacttcaacatcaaccttaacatcatcgaactccccttcactagcaccgaccacggtgtcccacccaacaccgaaaataccgataaactctcattaacctccatcatcaaactcttccatgcatccacttcactcgaacctcatgtacgtgactacctcacgcgccaccacctgcacgacccacccgtgtgcataatcttcgacgtgttcttgggctgggccgataacgtggcccgatcagttgggtccaccggaatctgcttcaacactggtggggcctacggtgtcggtgcgtatgtttcaatctggagcaatctacctcaccggaatgtcggcgatgatgaggagttttcattggctggtttcccggaagatcggaaactccgacgtaatcagcttcatcggtttttaaggtttgctgatgggtccgatgaatggtcgaggtttttccaacctcagattaaatcatccttgaattgttctgggtggttgtgtaattccatcgaggagattgaaccgttagggtttcaagtttttaggaatttcacgaaatctcctatttggggaattggtcctttgattatgacatcttcgaaaaaagatgatgatgagaaggaagaggcttgtttgaggtggttgaatcaatttgaaaatgactcagttttgtacatttgtttcgggtcacagaatacagtgactccgattcagatgatggagttagcgaaaggtttggaggagagtaagatcccgttcttgtgggtgattcggccgccattcgggttcgatttcaatggggagtttaaacccgaatggctgcctgagaagtttgaggaaagaatgatggagaaaaaacaggggatgttggtgagagattgggtgcctcagttggatattttgaggcatgaggctactggtggatttttgagtcattgtggttggaattcggtattggaaggtttaagagaaggagtgccaatactcgggtggccgttggcggccgagcaagcgtataattcaaagatgatggtggaggaaatgggggtggcggtcgagttgacgagggggttggaaggggaggtgaagaaagagtgggtgaagagtgtggtggaaatggtgttggatagaaaggaggggagttgtgggtgggaaatgaagaagaaagctgttgagattggggagaaattgaaggatgcttggaaaattgagggagattacaaaggatcttctgttaaggcaatggataattttgtagagtttatagtttcttgtaggggaaagaaatctaaagaagatgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaattcgcatgcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgatatcactatcctcttttcatccatcaagcacaactgctaccgctaccgcagctgccgcaaccacacaccctccacagcaatgccccgcattctcttcaccaccttcgcatctatccttgccgctacgacaccctcgtcaacatcttgtggttaggtgtggagggggtggctctgcatcggagtccgtattcaacagagattcagccgcaactagggtttcaaacgatcacttggatgtatctaaaagggatgtaaaactcaaaattgctatcatagggttcggaaactttgggcaatttctcgcaaagactatggcaaaacaaggtcatagagtactagcatattctcgatcagactattcccgggcggcaaaagagattggagttgaatattttactgatgcagatgacttgtgtgaggaacatcctgaagtaattcttttgtgtacatctatactgtctactgagaaggtgttgcggtcattacctctccatagattgcgcagatcaaccctgttcgctgatgttttgagtgtgaaagagttccctcgctctttatttctgcaactcttgcccaaagattttgatatactctgtacccaccctatgtttgggccagacagcgggaaagacgggtggggcggactcccattcgtatttgacaaagtaagggtgggcagcgaccaatctcggacgtcaagagctgaggcatttttagatgtttttagaaacgccgggtgtagaatggtggaaatgtcatgcgtagatcatgacaaacatgccgcaggctcacaatttattacacacatgatgggaagggtactggaaaaactagcacttgaaaatacgccaattaacacaaagggatacgaatcattactaaatctagttgataacacagcacgcgacagctttgaattgttttacggcctattcctttataacaagaatgctatggagcagttggatagaatggactgggcttttgagatggtgaaaaagcagctaagcggttatttgcatgatctggtgaggaaacaactaatgctggaagggaacaatgatcaggcggaggtcaccttcgataagcccctgatgctaccctctcccacaattaacccacctcaaatagtgccctccgctgacatggctgagaaaaagcacgatcttgtagttgtcaatgggactaggtaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgcttgtgtgagaccgaggatgcacatgtgaccgagggacacgaa

SEQ ID NO: 192 - DNA, 12973 bp, unidentified
  misc_feature 1..12973: note = PL-454 construct
  source 1..12973: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggaccatgcgacccttgcaatgatactagccatttggttcatcagttttcacttcattaaactactctttagccagcaaaccactaaactcctaccaccgggtcccaaaccattgccaatcataggaaacatccttgaggtgggaaagaaaccacaccggtcgttcgctaatcttgctaaaattcacgggccattaattagtcttagactcgggtcagtgactacaatagttgtgagctctgccgatgttgccaaagaaatgtttctaaagaaagatcatcctctaagtaatcgtaccatacccaacagcgttactgcgggtgatcatcacaaactaaccatgtcgtggttaccagtgtctcccaagtggaggaattttcggaaaataaccgctgtgcacttgctcagtccccaacggctcgacgcatgtcaaacatttagacatgccaaagtccaacagctatacgaatatgttcaagaatgcgctcagaagggtcaagcagtagacatcggtaaggcagctttcactacaagtcttaacctactttcaaaattatttttctcagtcgagttggctcaccataaaagtcagacatcacaagaattcaaggagctaatctggaacataatggaagatatcggtaaacccaactatgcagattattttcctatattgggctgcgtcgaccccagtggtatacggagacgattggcatgttcatttgacaaactcattgctgtttttcagtctataatttgcgaaagactagctccggatagttccacagcaactacaaccacaactgacgatgtgcttgatgtgttgctgcagttgttcaagcaaaatgagttaacaatgggtgagattaatcaccttctggtggatatattcgacgccggtactgatactacatcgtctacttttgagtgggtgatggctgagcttatcaggaacccagaaatgatggaaaaagcacaagaagagataaaacaagtgttggggaaagataaacaaatccaagagagtgatataatcaacttgccttatctgcaagcaataatcaaggagacactacgtctacaccctcccacagtctttctcttaccacggaaagccgacaccgacgtggaactgtacggatatatagttccgaaagatgctcaaattttagtcaacctttgggccattgggcgcgatcccaatgcctggcagaatgccgatatttttagtccagagagatttatcgggtgtgaaatcgatgttaaaggtagagacttcggactcttacccttcggggcaggcagaaggatatgtccaggaatgaacttagccatccgaatgctaacactgatgctagcaacccttctccaattctttaattggaagctagaaggtgacatctcgcccaaagaccttgacatggacgagaaatttggaattgctcttcaaaaaacaaaacccttaaaacttatacccattcccagatattaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgactgcaatcaaaatgaatacgaatggtgagggtgaaacccaacatatacttatgataccatttatggcccaagggcaccttcgaccgtttctggaattggccatgttcttatacaaacgatcacatgtcatcataactctgctaacaactccccttaacgctgggttcctgagacatctgttgcatcaccattcctacagctcctctgggatacggatcgtagaattgccgtttaattcgactaatcacgggctcccacccggtatagaaaatacagacaaattaacactgcctttagtcgtatccttgtttcattctacaatttccctagacccccacctacgcgattatatttcgagacacttcagtcctgcacgacccccactgtgtgttattcacgatgtttttctcggttgggtggaccaagtagctaaggatgtagggtccacaggcgtcgtttttacgaccggcggggcttatggcacctctgcatatgtatctatatggaatgacttaccacaccaaaattactccgacgatcaggagtttccattgccaggattccccgagaatcataaatttcgaagatctcaactgcatcgtttccttaggtatgctgatgggtctgatgactggtctaaatatttccaaccccaactacggcaaagtatgaaaagttttgggtggctatgcaatagtgtggaagagattgaaacattgggcttctcgattctaagaaactacaccaagcttccaatctggggcataggcccacttatagcctccccggtacagcacagttcaagtgacaataactctactggagcagagtttgtgcagtggttgagtcttaaagaacccgacagtgtattatatatatctttcgggagtcaaaatactatttcaccaactcagatgatggaattggctgcagggttggagtcgagtgagaaaccgtttctgtgggttatacgtgccccatttgggtttgatattaatgaggaaatgcgtcccgaatggcttcccgaggggtttgaagagaggatgaaagtcaaaaagcaagggaagcttgtttacaaattggggcctcaattggaaatccttaatcacgaaagtatcgggggttttctaacgcattgcgggtggaactctattcttgagtccctaagagaaggtgttcccatgttgggatggccactagcagctgaacaagcttacaacctaaaatatctagaggatgaaatgggtgtagcagtcgaactagccagaggcctcgagggggaaatttccaaagaaaaggttaagagaatagttgagatgatcttagagcggaatgaaggttcaaaaggatgggagatgaaaaacagggcagtggaaatggggaaaaagttaaaagatgccgtcaacgaggaaaaagagttgaaaggttcttcagtcaaagcaatagacgatttccttgacgctgtgatgcaagcaaagctcgaaccgtcattgcaatgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 193 - DNA, 12907 bp, unidentified
  misc_feature 1..12907: note = PL-455 construct
  source 1..12907: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggaccatgcgacccttgcaatgatactagccatttggttcatcagttttcacttcattaaactactctttagccagcaaaccactaaactcctaccaccgggtcccaaaccattgccaatcataggaaacatccttgaggtgggaaagaaaccacaccggtcgttcgctaatcttgctaaaattcacgggccattaattagtcttagactcgggtcagtgactacaatagttgtgagctctgccgatgttgccaaagaaatgtttctaaagaaagatcatcctctaagtaatcgtaccatacccaacagcgttactgcgggtgatcatcacaaactaaccatgtcgtggttaccagtgtctcccaagtggaggaattttcggaaaataaccgctgtgcacttgctcagtccccaacggctcgacgcatgtcaaacatttagacatgccaaagtccaacagctatacgaatatgttcaagaatgcgctcagaagggtcaagcagtagacatcggtaaggcagctttcactacaagtcttaacctactttcaaaattatttttctcagtcgagttggctcaccataaaagtcagacatcacaagaattcaaggagctaatctggaacataatggaagatatcggtaaacccaactatgcagattattttcctatattgggctgcgtcgaccccagtggtatacggagacgattggcatgttcatttgacaaactcattgctgtttttcagtctataatttgcgaaagactagctccggatagttccacagcaactacaaccacaactgacgatgtgcttgatgtgttgctgcagttgttcaagcaaaatgagttaacaatgggtgagattaatcaccttctggtggatatattcgacgccggtactgatactacatcgtctacttttgagtgggtgatggctgagcttatcaggaacccagaaatgatggaaaaagcacaagaagagataaaacaagtgttggggaaagataaacaaatccaagagagtgatataatcaacttgccttatctgcaagcaataatcaaggagacactacgtctacaccctcccacagtctttctcttaccacggaaagccgacaccgacgtggaactgtacggatatatagttccgaaagatgctcaaattttagtcaacctttgggccattgggcgcgatcccaatgcctggcagaatgccgatatttttagtccagagagatttatcgggtgtgaaatcgatgttaaaggtagagacttcggactcttacccttcggggcaggcagaaggatatgtccaggaatgaacttagccatccgaatgctaacactgatgctagcaacccttctccaattctttaattggaagctagaaggtgacatctcgcccaaagaccttgacatggacgagaaatttggaattgctcttcaaaaaacaaaacccttaaaacttatacccattcccagatattaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggattcatcaaaaacaaagcacattatcctcttgccatttctagcgcaaggtcatctccgtccatttctgcacttagccaatttcctcagatcccacactcctttcaccctttctatccttactactcctctcaatgccgccaatctccgccgtcaatccaacaacattaacatctacgatctaccctttaacagcaccgatcataatctaccaccaaacacagaaaacactgaaaaactcccattaacttcaataatctctctgttttatgcatccacttcgctccagcctcacgtacgcaaccaccttactcgccatcaccttgataacccacccatttgtatcatctttgatattttcttgggctgggctgataacttggcccgatctattggatctaccggagtttgtttcaacactggtggggcctacggtttggccgcttacatgtcgatttggatgcatttgcctcatagaaatgttccagacaatgtcgagttttcccttccggaattccctgagaatcggaaatttaaacgtaaccaacttcacaggtttttaagatttgctgatggaaccgatgattggtcgggtttttttcaaccccagataaagtactccaggaattgttctgcgtggttgtgtaattcaattgaggaaattgagccattagggtttgaggttttaagagcaatgttgaaagtaccagtttggggaattggtcctttagtcaagactgaaaatagttgtgatgaagatgatgaacaaggttgtgttgagtggttaaaccaatttgagaaaggatctgttttgtacatatcatttgggtcacagaatacagttaccccgatccaaatgatggaattagcaaaaggattggaggaaagtggggcgaaatttttgtgggtgattcgtccccctttcgggtttgatatcaatggagagttcaaacccgaatggctgccggagggatttgagaagagagtaatggaaaggaagcaggggaagctagtaaagaaatgggggcctcaaatggagatattgaggaataaggcgacgggggcgtttttgagtcactgtgggtggaattcgttgatcgaagcgttaagcgaaggtgttccgataatagggtggccattggctgcggagcaagcgtataattcaaagatgttagtggaagagatgggggttgcggtcgagttaacgaggggattggaaggggagttgagtagcgaaggggtgaagaaggtggtggagatggttatggatagggaagaagggagttttgggtgtgagatgaagaaggcggcagttgtaattggacagaaattgagggatgcaatgaaagtggaaggtgattatagagggtgttctcttagatctcttgatgagtttgtagagttcattgtttctcgtaaggctactttgagctaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 194 - DNA, 12946 bp, unidentified
  misc_feature 1..12946: note = PL-456 construct
  source 1..12946: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggaccatgcgacccttgcaatgatactagccatttggttcatcagttttcacttcattaaactactctttagccagcaaaccactaaactcctaccaccgggtcccaaaccattgccaatcataggaaacatccttgaggtgggaaagaaaccacaccggtcgttcgctaatcttgctaaaattcacgggccattaattagtcttagactcgggtcagtgactacaatagttgtgagctctgccgatgttgccaaagaaatgtttctaaagaaagatcatcctctaagtaatcgtaccatacccaacagcgttactgcgggtgatcatcacaaactaaccatgtcgtggttaccagtgtctcccaagtggaggaattttcggaaaataaccgctgtgcacttgctcagtccccaacggctcgacgcatgtcaaacatttagacatgccaaagtccaacagctatacgaatatgttcaagaatgcgctcagaagggtcaagcagtagacatcggtaaggcagctttcactacaagtcttaacctactttcaaaattatttttctcagtcgagttggctcaccataaaagtcagacatcacaagaattcaaggagctaatctggaacataatggaagatatcggtaaacccaactatgcagattattttcctatattgggctgcgtcgaccccagtggtatacggagacgattggcatgttcatttgacaaactcattgctgtttttcagtctataatttgcgaaagactagctccggatagttccacagcaactacaaccacaactgacgatgtgcttgatgtgttgctgcagttgttcaagcaaaatgagttaacaatgggtgagattaatcaccttctggtggatatattcgacgccggtactgatactacatcgtctacttttgagtgggtgatggctgagcttatcaggaacccagaaatgatggaaaaagcacaagaagagataaaacaagtgttggggaaagataaacaaatccaagagagtgatataatcaacttgccttatctgcaagcaataatcaaggagacactacgtctacaccctcccacagtctttctcttaccacggaaagccgacaccgacgtggaactgtacggatatatagttccgaaagatgctcaaattttagtcaacctttgggccattgggcgcgatcccaatgcctggcagaatgccgatatttttagtccagagagatttatcgggtgtgaaatcgatgttaaaggtagagacttcggactcttacccttcggggcaggcagaaggatatgtccaggaatgaacttagccatccgaatgctaacactgatgctagcaacccttctccaattctttaattggaagctagaaggtgacatctcgcccaaagaccttgacatggacgagaaatttggaattgctcttcaaaaaacaaaacccttaaaacttatacccattcccagatattaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggagtcatcaacaaaagaacaagaacaacaacacataatcctcctcccatttttagcacaaggccatctccgaccattcctccacctcgctcaccgccttctctctctaacacccttcaatctctctctcctcactacccctttaaacgccgctaatctccgccgtcaatccgacaacttcaacatcaaccttaacatcatcgaactccccttcactagcaccgaccacggtgtcccacccaacaccgaaaataccgataaactctcattaacctccatcatcaaactcttccatgcatccacttcactcgaacctcatgtacgtgactacctcacgcgccaccacctgcacgacccacccgtgtgcataatcttcgacgtgttcttgggctgggccgataacgtggcccgatcagttgggtccaccggaatctgcttcaacactggtggggcctacggtgtcggtgcgtatgtttcaatctggagcaatctacctcaccggaatgtcggcgatgatgaggagttttcattggctggtttcccggaagatcggaaactccgacgtaatcagcttcatcggtttttaaggtttgctgatgggtccgatgaatggtcgaggtttttccaacctcagattaaatcatccttgaattgttctgggtggttgtgtaattccatcgaggagattgaaccgttagggtttcaagtttttaggaatttcacgaaatctcctatttggggaattggtcctttgattatgacatcttcgaaaaaagatgatgatgagaaggaagaggcttgtttgaggtggttgaatcaatttgaaaatgactcagttttgtacatttgtttcgggtcacagaatacagtgactccgattcagatgatggagttagcgaaaggtttggaggagagtaagatcccgttcttgtgggtgattcggccgccattcgggttcgatttcaatggggagtttaaacccgaatggctgcctgagaagtttgaggaaagaatgatggagaaaaaacaggggatgttggtgagagattgggtgcctcagttggatattttgaggcatgaggctactggtggatttttgagtcattgtggttggaattcggtattggaaggtttaagagaaggagtgccaatactcgggtggccgttggcggccgagcaagcgtataattcaaagatgatggtggaggaaatgggggtggcggtcgagttgacgagggggttggaaggggaggtgaagaaagagtgggtgaagagtgtggtggaaatggtgttggatagaaaggaggggagttgtgggtgggaaatgaagaagaaagctgttgagattggggagaaattgaaggatgcttggaaaattgagggagattacaaaggatcttctgttaaggcaatggataattttgtagagtttatagtttcttgtaggggaaagaaatctaaagaagatgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 195 - DNA, 12901 bp, unidentified
  misc_feature 1..12901: note = PL-461 construct
  source 1..12901: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggataatgcaaccctagcaatgatacttacaatatggctcatttcaattaatttcataaaaatgttttttactcaccaaaacacaaaacttattcttcctccgggtccaaaaccattaccaattattggcaacattctcgaagttgggaaaaaacctcatcgttcattcgcaaacctagccaaaatccacggccctttaatttccttacgtttaggaagtgttacaacaattgtcgtatcatcagcagaagttgccaaagaaatgttcttaaaaaaagaccagcctctctcaaaccgtaatgttcccaactccgtcactgctggtgatcaccacaaactaaccatgtcatggttacccgtatcacccaaatggagaaatttccgtaaaatcacagccgttcatttactttcacctctaagacttgatgcatgtcaatctcttagacaagccaaggtgcaacaactttatcaatacgtacaagaatgtgctcaaaaaggacaatcaattgatatcggtaaagctgcatttacaacatcgttaaatcttttatcaaaactatttttttcaaaggaattagcttgccataaatctcatgaatctcaagaatttaaacaattaatttggaatataatggaagatattggtaaacctaattatgcagattattttccaatattaggatgtattgatccattagggattagaagaagattagcagctaattttgataaattaattgctgtttttcaaactataatatctgaaaggcttgagaataataaaaacgcaaatgcaactaatgatgtgcttgatgttctgctgcaattgtacaaacaaaaggagctgagtatgggtgaaattaatcatctactcgtggatatttttgatgctggaacagatactacttcaagcacttttgaatgggttatgacagaattaattagaaatccaaatatgatggaaaaagctcaacaagaaattcaagaagtattaggtaaagatagacaaattcaagaatcggacattattaagttgccttacttacaagcccttattaaagaaacattacgattacatccaccaactgtatttctcttaccccgtaaagctgatatggacgttgaactatatggatatgttgtgcctaaagatgcacaaatactcgttaatttgtgggctattggaagagatcctcaagcatgggaaaaacctaatgcttttttacccgagagatttttaggttctgatgttgatgtaaaaggacgagatttcggacttttaccttttggagccggaaaaagaatatgtcccggaatgaatttggctattagaatgttgactttgatgttggctactttacttcaattttttgattggaaacttgaagaaggtatgaatccacaagatttagacatggatgaaaaatttgggattgctttacaaaagaccaagcctcttgaaatcattccatcccgtaggcattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggattcatcaaaaacaaagcacattatcctcttgccatttctagcgcaaggtcatctccgtccatttctgcacttagccaatttcctcagatcccacactcctttcaccctttctatccttactactcctctcaatgccgccaatctccgccgtcaatccaacaacattaacatctacgatctaccctttaacagcaccgatcataatctaccaccaaacacagaaaacactgaaaaactcccattaacttcaataatctctctgttttatgcatccacttcgctccagcctcacgtacgcaaccaccttactcgccatcaccttgataacccacccatttgtatcatctttgatattttcttgggctgggctgataacttggcccgatctattggatctaccggagtttgtttcaacactggtggggcctacggtttggccgcttacatgtcgatttggatgcatttgcctcatagaaatgttccagacaatgtcgagttttcccttccggaattccctgagaatcggaaatttaaacgtaaccaacttcacaggtttttaagatttgctgatggaaccgatgattggtcgggtttttttcaaccccagataaagtactccaggaattgttctgcgtggttgtgtaattcaattgaggaaattgagccattagggtttgaggttttaagagcaatgttgaaagtaccagtttggggaattggtcctttagtcaagactgaaaatagttgtgatgaagatgatgaacaaggttgtgttgagtggttaaaccaatttgagaaaggatctgttttgtacatatcatttgggtcacagaatacagttaccccgatccaaatgatggaattagcaaaaggattggaggaaagtggggcgaaatttttgtgggtgattcgtccccctttcgggtttgatatcaatggagagttcaaacccgaatggctgccggagggatttgagaagagagtaatggaaaggaagcaggggaagctagtaaagaaatgggggcctcaaatggagatattgaggaataaggcgacgggggcgtttttgagtcactgtgggtggaattcgttgatcgaagcgttaagcgaaggtgttccgataatagggtggccattggctgcggagcaagcgtataattcaaagatgttagtggaagagatgggggttgcggtcgagttaacgaggggattggaaggggagttgagtagcgaaggggtgaagaaggtggtggagatggttatggatagggaagaagggagttttgggtgtgagatgaagaaggcggcagttgtaattggacagaaattgagggatgcaatgaaagtggaaggtgattatagagggtgttctcttagatctcttgatgagtttgtagagttcattgtttctcgtaaggctactttgagctaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 196 - DNA, 12898 bp, unidentified
  misc_feature 1..12898: note = PL-462 construct
  source 1..12898: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgactaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagggaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtagaaaaagaaatgaatttcaatgaaaccttttttataacccatggaaacccaattttaacagttgaagattctcatcctttaagacctttttttgaaacttggaaacaaaatatattttctaagaaaccaaaggctattcttgtgatttcaggtcattgggaaactgattttccttctgttaatgctgttgatatcaatgatactatccatgattttgatgattaccctgatgctatgtaccagttgaaatatccagctccagggtcaatagaaatggcaaaaaaagtggaagaaatactgacaaaatcaggttacaaaacaataaacatggacaaaaaacgaggactagatcatggtgcatgggtaccattaatgtacatgtatccccaagcagacatcccagtttgtcaactatcagttcaacccaagttaagtggaacataccattataacttgggaagagctttggctcctttgaaacaagaaggtgtacttataattggttccggaagtgctactcatcctttggatgaaaccccacattttcataatggtgttgcaccttgggctcatgaatttcattcttggcttcattctgctcttataaatcgaaggattgaagaagtgaacacatatgaaagcaaagcaccaaattgggaattagcacatccattcccagaacatttttatcccttgcacattgtcatgggtgcagcaggagataattggaaggcagagcttattcatagcagttgggaccatggcaccttatatcatggtgcctacaagttcacttccaactaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggattcatcaaaaacaaagcacattatcctcttgccatttctagcgcaaggtcatctccgtccatttctgcacttagccaatttcctcagatcccacactcctttcaccctttctatccttactactcctctcaatgccgccaatctccgccgtcaatccaacaacattaacatctacgatctaccctttaacagcaccgatcataatctaccaccaaacacagaaaacactgaaaaactcccattaacttcaataatctctctgttttatgcatccacttcgctccagcctcacgtacgcaaccaccttactcgccatcaccttgataacccacccatttgtatcatctttgatattttcttgggctgggctgataacttggcccgatctattggatctaccggagtttgtttcaacactggtggggcctacggtttggccgcttacatgtcgatttggatgcatttgcctcatagaaatgttccagacaatgtcgagttttcccttccggaattccctgagaatcggaaatttaaacgtaaccaacttcacaggtttttaagatttgctgatggaaccgatgattggtcgggtttttttcaaccccagataaagtactccaggaattgttctgcgtggttgtgtaattcaattgaggaaattgagccattagggtttgaggttttaagagcaatgttgaaagtaccagtttggggaattggtcctttagtcaagactgaaaatagttgtgatgaagatgatgaacaaggttgtgttgagtggttaaaccaatttgagaaaggatctgttttgtacatatcatttgggtcacagaatacagttaccccgatccaaatgatggaattagcaaaaggattggaggaaagtggggcgaaatttttgtgggtgattcgtccccctttcgggtttgatatcaatggagagttcaaacccgaatggctgccggagggatttgagaagagagtaatggaaaggaagcaggggaagctagtaaagaaatgggggcctcaaatggagatattgaggaataaggcgacgggggcgtttttgagtcactgtgggtggaattcgttgatcgaagcgttaagcgaaggtgttccgataatagggtggccattggctgcggagcaagcgtataattcaaagatgttagtggaagagatgggggttgcggtcgagttaacgaggggattggaaggggagttgagtagcgaaggggtgaagaaggtggtggagatggttatggatagggaagaagggagttttgggtgtgagatgaagaaggcggcagttgtaattggacagaaattgagggatgcaatgaaagtggaaggtgattatagagggtgttctcttagatctcttgatgagtttgtagagttcattgtttctcgtaaggctactttgagctaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 197 - DNA, 17511 bp, unidentified
  misc_feature 1..17511: note = PL-463 construct
  source 1..17511: mol_type = unassigned DNA
ttcgtgtccctcggtcacatgtgcatcctcggtctcatctgagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcaagcatcttctttagatttctttcccctacaagaaactataaactctacaaaattatccattgccttaacagaagatcctttgtaatctccctcaattttccaagcatccttcaatttctccccaatctcaacagctttcttcttcatttcccacccacaactcccctcctttctatccaacaccatttccaccacactcttcacccactctttcttcacctccccttccaaccccctcgtcaactcgaccgccacccccatttcctccaccatcatctttgaattatacgcttgctcggccgccaacggccacccgagtattggcactccttctcttaaaccttccaataccgaattccaaccacaatgactcaaaaatccaccagtagcctcatgcctcaaaatatccaactgaggcacccaatctctcaccaacatcccctgttttttctccatcattctttcctcaaacttctcaggcagccattcgggtttaaactccccattgaaatcgaacccgaatggcggccgaatcacccacaagaacgggatcttactctcctccaaacctttcgctaactccatcatctgaatcggagtcactgtattctgtgacccgaaacaaatgtacaaaactgagtcattttcaaattgattcaaccacctcaaacaagcctcttccttctcatcatcatcttttttcgaagatgtcataatcaaaggaccaattccccaaataggagatttcgtgaaattcctaaaaacttgaaaccctaacggttcaatctcctcgatggaattacacaaccacccagaacaattcaaggatgatttaatctgaggttggaaaaacctcgaccattcatcggacccatcagcaaaccttaaaaaccgatgaagctgattacgtcggagtttccgatcttccgggaaaccagccaatgaaaactcctcatcatcgccgacattccggtgaggtagattgctccagattgaaacatacgcaccgacaccgtaggccccaccagtgttgaagcagattccggtggacccaactgatcgggccacgttatcggcccagcccaagaacacgtcgaagattatgcacacgggtgggtcgtgcaggtggtggcgcgtgaggtagtcacgtacatgaggttcgagtgaagtggatgcatggaagagtttgatgatggaggttaatgagagtttatcggtattttcggtgttgggtgggacaccgtggtcggtgctagtgaaggggagttcgatgatgttaaggttgatgttgaagttgtcggattgacggcggagattagcggcgtttaaaggggtagtgaggagagagagattgaagggtgttagagagagaaggcggtgagcgaggtggaggaatggtcggagatggccttgtgctaaaaatgggaggaggattatgtgttgttgttcttgttcttttgttgatgactccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccccatgggaattcgtaaagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagcttagttggaagtgaacttgtaggcaccatgatataaggtgccatggtcccaactgctatgaataagctctgccttccaattatctcctgctgcacccatgacaatgtgcaagggataaaaatgttctgggaatggatgtgctaattcccaatttggtgctttgctttcatatgtgttcacttcttcaatccttcgatttataagagcagaatgaagccaagaatgaaattcatgagcccaaggtgcaacaccattatgaaaatgtggggtttcatccaaaggatgagtagcacttccggaaccaattataagtacaccttcttgtttcaaaggagccaaagctcttcccaagttataatggtatgttccacttaacttgggttgaactgatagttgacaaactgggatgtctgcttggggatacatgtacattaatggtacccatgcaccatgatctagtcctcgttttttgtccatgtttattgttttgtaacctgattttgtcagtatttcttccactttttttgccatttctattgaccctggagctggatatttcaactggtacatagcatcagggtaatcatcaaaatcatggatagtatcattgatatcaacagcattaacagaaggaaaatcagtttcccaatgacctgaaatcacaagaatagcctttggtttcttagaaaatatattttgtttccaagtttcaaaaaaaggtcttaaaggatgagaatcttcaactgttaaaattgggtttccatgggttataaaaaaggtttcattgaaattcatttctttttctaccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccgagctcgaattctagtagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcaatacctaagaacgggaataatctgaagaggtttagtcttttgtaaggcaatcccaaatttctcatccatgtctagatcttctgctttcataccttcttcaagcttccaattgaagaattgaagaagcgtagctaacatcaaagttaacattctaatagccaaattcatcccggggcatattctccttccagctccaaaaggtaagagtccaaaatctcttcctttcacatcaatttcggatcctaaaaacctctcaggtaaaaacacgtcaggtttcacccacgcttgagggtcccttccaatggcccataaattaacaagtatttgtgcatctttcggcacaacatagccatataattcaacatcactatcagctttacgaggcaagagaaatacagttggtgggtgtagccgcaatgtttctttgataatggcttgtaagtaaggtagcttaataatgtctgattcttgaatttgtctatccttgcccaagacttgctcaatttcttcttgagctttttccatcatcttaggatttcgaattaactctgccattacccattcaaaagtacttgatgtagtgtcagtcccagcatcaaaaatatccacgagcagatgatttatctcgcccatactgagctctttttgtttgtggaggtcaagaagaatgtctagcacatcatcggtcgtcttagtcgaagcagaatcctggccgttgccaatcctttgacatattatgctttgaaaaactgcaatcaacttgtcaaaattagacgccaatcgacgtcgaattcctgatgggtctaagcatcctaagattggaaaataatcagcgtagttaggtttgccaatatcttccataatattccatataagttctttgaattgttgagatgtatgggatttatggtgggctaattcgaccgaaaagaataattttgataacaaattaagggaggttgtaaatgctgccttgccaatatcaacagcttgtcctttttgtgcacattcttgtacatattggaaaagctgttgcactttggcatgtcttagggtttggcaagcatcaagtctttgaggagaaagtaaatgaacggcggtgatttttctaaagttccgccactttggagaaacaggcaaccacgacatagtcagcttatggtgatccccagcagtgacagaattaggaacagtgcggttagaaagtgggtggtctttttttaagaacatttctttggctacttcagccgaggatacaacaatagttgtgacacttcctagacgtaatgatatcaatggaccatgaatcttagcaagattagcaaatgactggtgaggtttctcaccaacttcaaggatgtttccgattagtggcaatggtttaggaccggggggaagaagtttggtagtttggctagtgaataacattttaatgaaatgaaaaacaacaaaccaaatagcaagtatcattgctagtgttgcatgatccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccaagcttgaattcttgcagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgagaagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtctgcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcctccggatccgaattcggcatgagacccacagagtgttcaaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcaaccatgactactgtccggtccaacagatccgagcaggggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcaagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggatctgccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtaatggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcgctgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcctcgggaattggccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcggctggcacatacaaatggacgaacggataaaccttttcacgcccttttaaatatccgattattctaataaacgctcttttctcttaggtttacccgccaatatatcctgtcaaacactgatagtttaaacggatcac

SEQ ID NO: 198 - DNA, 12907 bp, unidentified
  misc_feature 1..12907: note = PL-464 construct
  source 1..12907: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgactaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagggaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggattcatcaaaaacaaagcacattatcctcttgccatttctagcgcaaggtcatctccgtccatttctgcacttagccaatttcctcagatcccacactcctttcaccctttctatccttactactcctctcaatgccgccaatctccgccgtcaatccaacaacattaacatctacgatctaccctttaacagcaccgatcataatctaccaccaaacacagaaaacactgaaaaactcccattaacttcaataatctctctgttttatgcatccacttcgctccagcctcacgtacgcaaccaccttactcgccatcaccttgataacccacccatttgtatcatctttgatattttcttgggctgggctgataacttggcccgatctattggatctaccggagtttgtttcaacactggtggggcctacggtttggccgcttacatgtcgatttggatgcatttgcctcatagaaatgttccagacaatgtcgagttttcccttccggaattccctgagaatcggaaatttaaacgtaaccaacttcacaggtttttaagatttgctgatggaaccgatgattggtcgggtttttttcaaccccagataaagtactccaggaattgttctgcgtggttgtgtaattcaattgaggaaattgagccattagggtttgaggttttaagagcaatgttgaaagtaccagtttggggaattggtcctttagtcaagactgaaaatagttgtgatgaagatgatgaacaaggttgtgttgagtggttaaaccaatttgagaaaggatctgttttgtacatatcatttgggtcacagaatacagttaccccgatccaaatgatggaattagcaaaaggattggaggaaagtggggcgaaatttttgtgggtgattcgtccccctttcgggtttgatatcaatggagagttcaaacccgaatggctgccggagggatttgagaagagagtaatggaaaggaagcaggggaagctagtaaagaaatgggggcctcaaatggagatattgaggaataaggcgacgggggcgtttttgagtcactgtgggtggaattcgttgatcgaagcgttaagcgaaggtgttccgataatagggtggccattggctgcggagcaagcgtataattcaaagatgttagtggaagagatgggggttgcggtcgagttaacgaggggattggaaggggagttgagtagcgaaggggtgaagaaggtggtggagatggttatggatagggaagaagggagttttgggtgtgagatgaagaaggcggcagttgtaattggacagaaattgagggatgcaatgaaagtggaaggtgattatagagggtgttctcttagatctcttgatgagtttgtagagttcattgtttctcgtaaggctactttgagctaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

SEQ ID NO: 199 - DNA, 12946 bp, unidentified
  misc_feature 1..12946: note = PL-465 construct
  source 1..12946: mol_type = unassigned DNA
tgaacactctgtgggtctcatgccgaattcggatccggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggatcatgcaacactagcaatgatacttgctatttggtttgttgtttttcatttcattaaaatgttattcactagccaaactaccaaacttcttccccccggtcctaaaccattgccactaatcggaaacatccttgaagttggtgagaaacctcaccagtcatttgctaatcttgctaagattcatggtccattgatatcattacgtctaggaagtgtcacaactattgttgtatcctcggctgaagtagccaaagaaatgttcttaaaaaaagaccacccactttctaaccgcactgttcctaattctgtcactgctggggatcaccataagctgactatgtcgtggttgcctgtttctccaaagtggcggaactttagaaaaatcaccgccgttcatttactttctcctcaaagacttgatgcttgccaaaccctaagacatgccaaagtgcaacagcttttccaatatgtacaagaatgtgcacaaaaaggacaagctgttgatattggcaaggcagcatttacaacctcccttaatttgttatcaaaattattcttttcggtcgaattagcccaccataaatcccatacatctcaacaattcaaagaacttatatggaatattatggaagatattggcaaacctaactacgctgattattttccaatcttaggatgcttagacccatcaggaattcgacgtcgattggcgtctaattttgacaagttgattgcagtttttcaaagcataatatgtcaaaggattggcaacggccaggattctgcttcgactaagacgaccgatgatgtgctagacattcttcttgacctccacaaacaaaaagagctcagtatgggcgagataaatcatctgctcgtggatatttttgatgctgggactgacactacatcaagtacttttgaatgggtaatggcagagttaattcgaaatcctaagatgatggaaaaagctcaagaagaaattgagcaagtcttgggcaaggatagacaaattcaagaatcagacattattaagctaccttacttacaagccattatcaaagaaacattgcggctacacccaccaactgtatttctcttgcctcgtaaagctgatagtgatgttgaattatatggctatgttgtgccgaaagatgcacaaatacttgttaatttatgggccattggaagggaccctcaagcgtgggtgaaacctgacgtgtttttacctgagaggtttttaggatccgaaattgatgtgaaaggaagagattttggactcttaccttttggagctggaaggagaatatgccccgggatgaatttggctattagaatgttaactttgatgttagctacgcttcttcaattcttcaattggaagcttgaagaaggtatgaaagcagaagatctagacatggatgagaaatttgggattgccttacaaaagactaaacctcttcagattattcccgttcttaggtattgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatgaaaatgagtggagaagataatagcatgatcaaagaaactttcttcataacccatggaaacccgattttgaccgtagaagatacacatcccttaaggccattctttgaaacatggagagaaaaaatctttacgatgaagccgaaggcgatacttgtgatctctggtcactgggaaactgatcatcctgctgttaatgctgttcatatcaatgatactatctacgatttcgatgactatcctcaagccatgtacaagttgaagtatccagctccaggatcaccagacttggctagcagagtagaagaaattatgaaaaaatccgggttcgacacagtgcacattgacaaaaaacgtgggcttgatcatggtgcatgggtgcctcttatgtacatgtatccggaggctgacataccggtatgtcagctctccgtccagccgaagttggacggaacgtaccactacaacttgggacgagcattagcgcctctaagagacgaaggtgtactcattattggttccggaagtgcaacacaccctttggacgaaacccctcattatcatggtggtgttgctccttgggctgctgactttgactcatggcttgacgtagctctcactaaaggaagatttgaagaagtgaatacatatgaaaccaaagcaccaaactgggaattggcacatccattcccggagcatttttatccattgcatgtcgtggttggggcggctggtgaaaagtggaaggctgagcttattcatactagttgggaccatggtaccttatgtcatggctcctacaagtttacttccacctaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggaagccaactaaacaagaccataaccatggtgacatttgacatagttgtttactacttgcttgagccccacccttgcttatcggttgaacattacaagatacactgcgggtggcctaaggcacaccgtccgaaaccggcaaaccaagcctgatcgccgaaatccaaaatcactaccggcaatctctaaagtttatttcatccttatatgacgaggaaagaaaagaagagagaaataatatcttaacttctaaatcagtcgcgtcaactttctcggctaagaaagtgagcactatcatttcgcagaccatgtcatgagtgccgacttgccatatcttattatattcttatttatttaattataatcccattgcaatacgtctattctatcatggcctgccactaacgctccgtctaacgtcgttaagccattgtcataagcggctgctcaaaactcttcccggtggaggcgaggcgttaacggcgtctacaaatctaacggccaccaaccatccagccgcctctcgaaagctccgctccgatcgcggaaattgcgtggcggagacgagcgggctcctctcacacggcccggaaccgtcacggcacgggtgggggattccttccccaaccctccccacctctcctccccccgtcgcagcccataaatacagggccctccgcgcctcttcccacaatctcacatcgtctcatcgttcggagcgcacaacccccgggttccaaatccaaattgctcttctcgcgaccctcggcgatccttcccccgcttcaaggtacggcgatcgtctcccccgtcctcttgccccatctcctcgctcggcgtggtttggtggttctgcttggtctgtggctaggaactaggctgaggcgttgacgaaatcatgctagatccgcgtgtttcctgatcgtgggtggctgggaggtggggttttcgtgtagatctgatcggttccgctgtttatcctgtcatgctcatgtgatttgtggggattttaggtcgtttgtccgggaatcgtggggttgcttctaggctgttcgtagatgagatcgttctcacgatctgctgggtcgctgcctaggttcagctaggtctgccctgtttttgggttcgttttcgggatctgtacgtgcatctattatctggttcgatggtgctagctaggaacaaacaactgattcgtccgatcgattgttttgttgccatgtgcaaggttaggtcgttatctgattgctgtagatcagagtagaataagatcatcacaagctagctcttgggcttattatgaatctgcgtttgttgcatgattaagatgattatgctttttcttatgctgccgtttgtatatgatgcggtagcttttaactgaatagcacacctttcctgtttagttagattagattagattgcatgatagatgaggatatatgctgctacatcagtttgatgattctctggtacctcataatcaactagctcatgtgcttaaattgaaactgcatgtgccacatgattaagatgctaagattggtgaagatatatacgctgctgttcctataggatcctgtagcttttacctggtcaacatgcatcgtcctgttatggatagatatgcatgatagatgaagatatgtactgctacaatttgatgattcttttgtgcacctgatgatcatgcatgctctttgcccttactttgatatacttggatgatggcatgcttagtactaatgatgtgatgaacacacatgacctgttggtatgaatatgatgttgctgtttgcttgtgatgagttctgtttgtttactgctaggcacttaccctgttgtctggttctcttttgcagaatggagtcatcaacaaaagaacaagaacaacaacacataatcctcctcccatttttagcacaaggccatctccgaccattcctccacctcgctcaccgccttctctctctaacacccttcaatctctctctcctcactacccctttaaacgccgctaatctccgccgtcaatccgacaacttcaacatcaaccttaacatcatcgaactccccttcactagcaccgaccacggtgtcccacccaacaccgaaaataccgataaactctcattaacctccatcatcaaactcttccatgcatccacttcactcgaacctcatgtacgtgactacctcacgcgccaccacctgcacgacccacccgtgtgcataatcttcgacgtgttcttgggctgggccgataacgtggcccgatcagttgggtccaccggaatctgcttcaacactggtggggcctacggtgtcggtgcgtatgtttcaatctggagcaatctacctcaccggaatgtcggcgatgatgaggagttttcattggctggtttcccggaagatcggaaactccgacgtaatcagcttcatcggtttttaaggtttgctgatgggtccgatgaatggtcgaggtttttccaacctcagattaaatcatccttgaattgttctgggtggttgtgtaattccatcgaggagattgaaccgttagggtttcaagtttttaggaatttcacgaaatctcctatttggggaattggtcctttgattatgacatcttcgaaaaaagatgatgatgagaaggaagaggcttgtttgaggtggttgaatcaatttgaaaatgactcagttttgtacatttgtttcgggtcacagaatacagtgactccgattcagatgatggagttagcgaaaggtttggaggagagtaagatcccgttcttgtgggtgattcggccgccattcgggttcgatttcaatggggagtttaaacccgaatggctgcctgagaagtttgaggaaagaatgatggagaaaaaacaggggatgttggtgagagattgggtgcctcagttggatattttgaggcatgaggctactggtggatttttgagtcattgtggttggaattcggtattggaaggtttaagagaaggagtgccaatactcgggtggccgttggcggccgagcaagcgtataattcaaagatgatggtggaggaaatgggggtggcggtcgagttgacgagggggttggaaggggaggtgaagaaagagtgggtgaagagtgtggtggaaatggtgttggatagaaaggaggggagttgtgggtgggaaatgaagaagaaagctgttgagattggggagaaattgaaggatgcttggaaaattgagggagattacaaaggatcttctgttaaggcaatggataattttgtagagtttatagtttcttgtaggggaaagaaatctaaagaagatgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagatgagaccgaggatgcacatgtgaccgagggacacgaa

(19) United States
(12) Patent Application Publication
(10) Pub. No.: US 2026/0218226 A1
(43) Pub. Date: Jul. 30, 2026

(54) RED PLANTS

(71) Applicant: Neoplants SAS, Saint-Denis (FR)

(72) Inventors: Iman Tabatabaei, Saint-Ouen-sur-Seine (FR); Patrick Torbey, Saint-Ouen-sur-Seine (FR); Margaux D'Avola, Paris (FR); Dona Victoire Sleiman, Paris (FR); Jérôme Jean-Luc Sébastien Bodin, Presles (FR); Laura Cristina P De Biolley, Paris (FR); Antoine Souvannasao, Saint-Ouen-Sur-Seine (FR); Sebastian Serban Cocioba, New York, NY (US)

(21) Appl. No.: 19/464,281

(22) Filed: Jan. 29, 2026

Related U.S. Application Data
(60) Provisional application No. 63/751,269, filed on Jan. 29, 2025.

(51) Int. Cl. C12N 15/82

(52) U.S. Cl. CPC C12N 15/8289

(57) ABSTRACT

The present disclosure provides technologies for engineering of pigments (e.g., betalain) in organisms to exhibit one or more colors and/or patterns (e.g., one or more non-natural colors and/or patterns). In some embodiments, such organisms are chloroplast-containing and/or photosynthetic organisms (CPOS). In some embodiments, such organisms are plants (e.g., indoor plants, outdoor plants, ornamental plants, etc).

### CROSS-REFERENCE TO RELATED APPLICATIONS

This application claims priority to and benefit of U.S. Provisional Application No. 63/751,269 filed Jan. 29, 2025, the entirety which is incorporated herein by reference.

### BACKGROUND

Indoor and ornamental plants make important and valuable contributions to our environments.

### SEQUENCE LISTING

In accordance with 37 C.F.R. § 1.834 (c) (1), a Sequence Listing in the form of an XML file (entitled “2013810-0052.xml” created on Feb. 18, 2026, and 627,267 bytes in size) is incorporated herein by reference in its entirety.

### SUMMARY

The present disclosure recognizes certain challenges associated with engineering organisms to exhibit one or more heterologous colors and/or patterns (e.g., one or more non-natural colors and/or patterns). For example, certain regulatory elements need to be assessed to control expression or a level of one or more engineered polypeptides (e.g., polypeptides that themselves produce one or more colors and/or patterns (e.g., one or more non-natural colors and/or patterns) or enzymes that catalyze reactions that produce one or more colors and/or patterns (e.g., one or more non-natural colors and/or patterns)) or genes (e.g., genes encoding polypeptides that themselves produce one or more colors and/or patterns (e.g., one or more non-natural colors and/or patterns) or enzymes that catalyze reactions that produce one or more colors and/or patterns (e.g., one or more non-natural colors and/or patterns)).

The present disclosure provides technologies for engineering particular organisms to exhibit one or more colors and/or patterns (e.g., one or more non-natural colors and/or patterns). In particular, the present disclosure provides technologies for engineering photosynthetic and/or chloroplast-containing organisms, such as plants (e.g., indoor plants and/or ornamental plants) to exhibit one or more colors and/or patterns (e.g., one or more non-natural colors and/or patterns). For simplicity, the present disclosure will use the term “CPOS”, for “chlorophyll-containing and/or photosynthetic organisms”, to refer to organisms to which teachings of the present disclosure relate. In many embodiments, CPOS are plants; in particular embodiments, CPOS are ornamental and/or indoor plants.

In some embodiments, the present disclosure recognizes advantages of engineered CPOS expressing certain polypeptides or genes associated with certain biosynthetic pathways (e.g., betalain biosynthesis pathway). In some embodiments, provided technologies include and/or utilize engineered polypeptides (e.g., polypeptides that themselves produce a red and/or purple color and/or pattern or enzymes that catalyze reactions that produce a red and/or purple color and/or pattern), genes, CPOS (e.g., plants, and/or microorganisms (e.g., in the plant biome)) and/or technologies for developing, producing, and/or utilizing them. In some embodiments, provided technologies includes systems (e.g., methods and/or components) for cultivating plants and/or associated organisms (e.g., microorganisms for example that may participate in a plant microbiome.

Among other things, the present disclosure provides a construct comprising a coding sequence. In some embodiments, a coding sequence comprises one or more nucleic acid sequences that each encode a pigment-producing polypeptide. In some embodiments, a coding sequence comprises a first nucleic acid sequence encoding a first pigment-producing polypeptide. In some embodiments, a coding sequence comprises a second nucleic acid sequence encoding a second pigment-producing polypeptide. In some embodiments, a coding sequence comprises a third nucleic acid sequence encoding a third pigment-producing polypeptide. In some embodiments, a construct comprises a coding sequence comprising (i) a first nucleic acid sequence encoding a first pigment-producing polypeptide, (ii) a second nucleic acid sequence encoding a second pigment-producing polypeptide, and (iii) a third nucleic acid sequence encoding a third pigment-producing polypeptide.

In some embodiments, a construct comprises one or more coding sequences. In some embodiments, a construct comprises a first coding sequence and a second coding sequence. In some embodiments, a first coding sequence comprises a first nucleic acid sequence encoding a first pigment-producing polypeptide. In some embodiments, a first coding sequence comprises a second nucleic acid sequence encoding a second pigment-producing polypeptide. In some embodiments, a first coding sequence comprises a third nucleic acid sequence encoding a third pigment-producing polypeptide. In some embodiments, a first coding sequence comprises (i) a first nucleic acid sequence encoding a first pigment-producing polypeptide, (ii) a second nucleic acid sequence encoding a second pigment-producing polypeptide, and (iii) a third nucleic acid sequence encoding a third pigment-producing polypeptide. In some embodiments, a second coding sequence comprises a fourth nucleic acid sequence encoding a fourth pigment-producing polypeptide. In some embodiments, a second coding sequence comprises a fifth nucleic acid sequence encoding a fifth pigment-producing polypeptide. In some embodiments, a second coding sequence comprises (i) a fourth nucleic acid sequence encoding a fourth pigment-producing polypeptide and (ii) a fifth nucleic acid sequence encoding a fifth pigment-producing polypeptide. In some embodiments, a construct comprises a coding sequence comprising (i) the first coding sequence comprises: (a) a first nucleic acid sequence encoding a first pigment-producing polypeptide, (b) a second nucleic acid sequence encoding a second pigment-producing polypeptide, and (c) a third nucleic acid sequence encoding a third pigment-producing polypeptide; and (ii) the second coding sequence comprises: (a) a fourth nucleic acid sequence encoding a fourth pigment-producing polypeptide, and (b) a fifth nucleic acid sequence encoding a fifth pigment-producing polypeptide.

In some embodiments, a first pigment-producing polypeptide is or comprises a cytochrome P450 (CYP) polypeptide (e.g., a CYP76AD1 polypeptide, a CYP76AD5 polypeptide, a CYP76AD6 polypeptide, or a CYP76AD15 polypeptide). In some embodiments, a first pigment-producing polypeptide is or comprises a cyclo-DOPA 5-O-glucosyltransferase (cDOPA5GT) polypeptide. In some embodiments, a first pigment-producing polypeptide is or comprises a L-DOPA 4,5-dioxygenase (DODA) polypeptide. In some embodiments, a first pigment-producing polypeptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12.

In some embodiments, a second pigment-producing polypeptide is or comprises a cytochrome P450 (CYP) polypeptide (e.g., a CYP76AD1 polypeptide, a CYP76AD5 polypeptide, a CYP76AD6 polypeptide, or a CYP76AD15 polypeptide). In some embodiments, a second pigment-producing polypeptide is or comprises a cyclo-DOPA 5-O-glucosyltransferase (cDOPA5GT) polypeptide. In some embodiments, a second pigment-producing polypeptide is or comprises a L-DOPA 4,5-dioxygenase (DODA) polypeptide. In some embodiments, a second pigment-producing polypeptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17.

In some embodiments, a third pigment-producing polypeptide is or comprises a cytochrome P450 (CYP) polypeptide (e.g., a CYP76AD1 polypeptide, a CYP76AD5 polypeptide, a CYP76AD6 polypeptide, or a CYP76AD15 polypeptide). In some embodiments, a third pigment-producing polypeptide is or comprises a cyclo-DOPA 5-O-glucosyltransferase (cDOPA5GT) polypeptide. In some embodiments, a third pigment-producing polypeptide is or comprises a L-DOPA 4,5-dioxygenase (DODA) polypeptide. In some embodiments, a third pigment-producing polypeptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11.

In some embodiments, a fourth pigment-producing polypeptide is or comprises a 3-deoxy-D-arabino-heptulosonate 7-phosphate synthase (DHAP synthase) polypeptide (e.g., AroG^{L175Q}). In some embodiments, a fourth pigment-producing polypeptide is or comprises an arogenate dehydrogenase (ADH) polypeptide (e.g., ADHα). In some embodiments, a fourth pigment-producing polypeptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 133.

In some embodiments, a fifth pigment-producing polypeptide is or comprises a fifth pigment-producing polypeptide is or comprises a 3-deoxy-D-arabino-heptulosonate 7-phosphate synthase (DHAP synthase) polypeptide (e.g., AroG^{L175Q}). In some embodiments, a fifth pigment-producing polypeptide is or comprises an arogenate dehydrogenase (ADH) polypeptide (e.g., ADHα). In some embodiments, a fifth pigment-producing polypeptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 8.

In some embodiments, a construct comprises a coding sequence comprising (i) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 127, (ii) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 130, and (iii) a third nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 128.

In some embodiments, a construct comprises (i) a first coding sequence comprising (a) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 127, (b) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 130, (c) a third nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 128, and (ii) a second coding (a) the fourth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 129, and (b) the fifth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 132.

In some embodiments, a construct comprising one or more nucleic acid sequences that each encode a self-cleaving peptide. In some embodiments, a self-cleaving peptide is or comprises a T2A peptide. In some embodiments, a self-cleaving peptide is or comprises a P2A peptide. In some embodiments, a self-cleaving peptide is or comprises an E2A peptide. In some embodiments, a self-cleaving peptide is or comprises a F2A peptide. In some embodiments, a self-cleaving peptide is or comprises an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 149.

In some embodiments, a construct comprises a first nucleic acid sequence and second nucleic acid sequence separated by a first nucleic acid sequence that encodes a self-cleaving peptide (e.g., a first P2A peptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 149). In some embodiments, a construct further comprises a second nucleic acid sequence and third nucleic acid sequence separated by a second nucleic acid sequence that encodes a self-cleaving peptide (e.g., a first P2A peptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 149). In some embodiments, a first nucleic acid sequence that encodes a first self-cleaving peptide is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 93. In some embodiments, a second nucleic acid sequence that encodes a second self-cleaving peptide is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 147.

In some embodiments, a construct comprises a promoter. In some embodiments, a construct comprises a promoter operatively linked to a coding sequence. In some embodiments, a construct comprises one or more promoters. In some embodiments, a construct comprises a first promoter and second promoter. In some embodiments, a construct comprises a first promoter operatively linked to a first coding sequence and a second promoter operatively linked to a second coding sequence. In some embodiments, a promoter is or comprises an inducible promoter. In some embodiments, a promoter is or comprises a constitutive promoter. In some embodiments, a promoter is or comprises a plant tissue specific promoter. In some embodiments, a promoter is or comprises a plant cell specific promoter. In some embodiments, a promoter is or comprise a 2×CaMV 35S+TMV 5′UTR promoter. In some embodiments, a promoter is or comprise an AthAct2 promoter. In some embodiments, a promoter is or comprise an AthLHB1B1 promoter. In some embodiments, a promoter is or comprise a BdEF1a promoter. In some embodiments, a promoter is or comprise a BdUbi10 promoter. In some embodiments, a promoter is or comprise a CaMV 35S long promoter. In some embodiments, a promoter is or comprise a CaMV 35S short promoter. In some embodiments, a promoter is or comprise a CsVMV promoter. In some embodiments, a promoter is or comprise a Mas promoter. In some embodiments, a promoter is or comprise a Nos promoter. In some embodiments, a promoter is or comprise an Ocs promoter. In some embodiments, a promoter is or comprise an OsActin promoter. In some embodiments, a promoter is or comprise a PsSEOF1 promoter. In some embodiments, a promoter is or comprise a PvUbi1+3 promoter. In some embodiments, a promoter is or comprise a PvUbi2 promoter. In some embodiments, a promoter is or comprise a RolC promoter. In some embodiments, a promoter is or comprise an RSs1 promoter. In some embodiments, a promoter is or comprise an RTBV promoter. In some embodiments, a promoter is or comprise an rrEaAct1Blast promoter. In some embodiments, a promoter is or comprise an rrEaActBlast2 promoter. In some embodiments, a promoter is or comprise an rrEaAs2Blast1 promoter. In some embodiments, a promoter is or comprise an rrEaCons1 promoter. In some embodiments, a promoter is or comprise an rrEaCons2 promoter. In some embodiments, a promoter is or comprise an rrEaCons3 promoter. In some embodiments, a promoter is or comprise an rrEaCons4 promoter. In some embodiments, a promoter is or comprise an rrEaCons5 promoter. In some embodiments, a promoter is or comprise an rrEaCons6 promoter. In some embodiments, a promoter is or comprise an rrEaCons7 promoter. In some embodiments, a promoter is or comprise an rrEaCons8 promoter. In some embodiments, a promoter is or comprise an rrEaDPA4Blast1 promoter. In some embodiments, a promoter is or comprise an rrEaH3Blast2 promoter. In some embodiments, a promoter is or comprise an rrEaLeaf1 promoter. In some embodiments, a promoter is or comprise an rrEaLeaf4 promoter. In some embodiments, a promoter is or comprise an rrEaUbiBlast1 promoter. In some embodiments, a promoter is or comprise a prom7_V1343 promoter. In some embodiments, a promoter is or comprise a prom7_V2197 promoter. In some embodiments, a promoter is or comprise a prom7_V956 promoter. In some embodiments, a promoter is or comprise a SlHis4 promoter. In some embodiments, a promoter is or comprise a ZmUbi promoter. In some embodiments, a first promoter is or comprise a 2×CaMV 35S+TMV 5′UTR promoter. In some embodiments, a first promoter is or comprise an AthAct2 promoter. In some embodiments, a first promoter is or comprise an AthLHB1B1 promoter. In some embodiments, a first promoter is or comprise a BdEF1a promoter. In some embodiments, a first promoter is or comprise a BdUbi10 promoter. In some embodiments, a first promoter is or comprise a CaMV 35S long promoter. In some embodiments, a first promoter is or comprise a CaMV 35S short promoter. In some embodiments, a first promoter is or comprise a CsVMV promoter. In some embodiments, a first promoter is or comprise a Mas promoter. In some embodiments, a first promoter is or comprise a Nos promoter. In some embodiments, a first promoter is or comprise an Ocs promoter. In some embodiments, a first promoter is or comprise an OsActin promoter. In some embodiments, a first promoter is or comprise a PsSEOF1 promoter. In some embodiments, a first promoter is or comprise a PvUbi1+3 promoter. In some embodiments, a first promoter is or comprise a PvUbi2 promoter. In some embodiments, a first promoter is or comprise a RolC promoter. In some embodiments, a first promoter is or comprise an RSs1 promoter. In some embodiments, a first promoter is or comprise an RTBV promoter. In some embodiments, a first promoter is or comprise an rrEaAct1Blast promoter. In some embodiments, a first promoter is or comprise an rrEaActBlast2 promoter. In some embodiments, a first promoter is or comprise an rrEaAs2Blast1 promoter. In some embodiments, a first promoter is or comprise an rrEaCons1 promoter. In some embodiments, a first promoter is or comprise an rrEaCons2 promoter. In some embodiments, a first promoter is or comprise an rrEaCons3 promoter. In some embodiments, a first promoter is or comprise an rrEaCons4 promoter. In some embodiments, a first promoter is or comprise an rrEaCons5 promoter. In some embodiments, a first promoter is or comprise an rrEaCons6 promoter. In some embodiments, a first promoter is or comprise an rrEaCons7 promoter. In some embodiments, a first promoter is or comprise an rrEaCons8 promoter. In some embodiments, a first promoter is or comprise an rrEaDPA4Blast1 promoter. In some embodiments, a first promoter is or comprise an rrEaH3Blast2 promoter. In some embodiments, a first promoter is or comprise an rrEaLeaf1 promoter. In some embodiments, a first promoter is or comprise an rrEaLeaf4 promoter. In some embodiments, a first promoter is or comprise an rrEaUbiBlast1 promoter. In some embodiments, a first promoter is or comprise a prom7_V1343 promoter. In some embodiments, a first promoter is or comprise a prom7_V2197 promoter. In some embodiments, a first promoter is or comprise a prom7_V956 promoter. In some embodiments, a first promoter is or comprise a SlHis4 promoter. In some embodiments, a first promoter is or comprise a ZmUbi promoter. In some embodiments, a second promoter is or comprise a 2×CaMV 35S+TMV 5′UTR promoter. In some embodiments, a second promoter is or comprise an AthAct2 promoter. In some embodiments, a second promoter is or comprise an AthLHB1B1 promoter. In some embodiments, a second promoter is or comprise a BdEF1a promoter. In some embodiments, a second promoter is or comprise a BdUbi10 promoter. In some embodiments, a second promoter is or comprise a CaMV 35S long promoter. In some embodiments, a second promoter is or comprise a CaMV 35S short promoter. In some embodiments, a second promoter is or comprise a CsVMV promoter. In some embodiments, a second promoter is or comprise a Mas promoter. In some embodiments, a second promoter is or comprise a Nos promoter. In some embodiments, a second promoter is or comprise an Ocs promoter. In some embodiments, a second promoter is or comprise an OsActin promoter. In some embodiments, a second promoter is or comprise a PsSEOF1 promoter. In some embodiments, a second promoter is or comprise a PvUbi1+3 promoter. In some embodiments, a second promoter is or comprise a PvUbi2 promoter. In some embodiments, a second promoter is or comprise a RolC promoter. In some embodiments, a second promoter is or comprise an RSs1 promoter. In some embodiments, a second promoter is or comprise an RTBV promoter. In some embodiments, a second promoter is or comprise an rrEaAct1Blast promoter. In some embodiments, a second promoter is or comprise an rrEaActBlast2 promoter. In some embodiments, a second promoter is or comprise an rrEaAs2Blast1 promoter. In some embodiments, a second promoter is or comprise an rrEaCons1 promoter. In some embodiments, a second promoter is or comprise an rrEaCons2 promoter. In some embodiments, a second promoter is or comprise an rrEaCons3 promoter. In some embodiments, a second promoter is or comprise an rrEaCons4 promoter. In some embodiments, a second promoter is or comprise an rrEaCons5 promoter. In some embodiments, a second promoter is or comprise an rrEaCons6 promoter. In some embodiments, a second promoter is or comprise an rrEaCons7 promoter. In some embodiments, a second promoter is or comprise an rrEaCons8 promoter. In some embodiments, a second promoter is or comprise an rrEaDPA4Blast1 promoter. In some embodiments, a second promoter is or comprise an rrEaH3Blast2 promoter. In some embodiments, a second promoter is or comprise an rrEaLeaf1 promoter. In some embodiments, a second promoter is or comprise an rrEaLeaf4 promoter. In some embodiments, a second promoter is or comprise an rrEaUbiBlast1 promoter. In some embodiments, a second promoter is or comprise a prom7_V1343 promoter. In some embodiments, a second promoter is or comprise a prom7_V2197 promoter. In some embodiments, a second promoter is or comprise a prom7_V956 promoter. In some embodiments, a second promoter is or comprise a SlHis4 promoter. In some embodiments, a second promoter is or comprise a ZmUbi promoter.

In some embodiments, a construct comprises one or more terminators. In some embodiments, a construct comprises at least one terminator. In some embodiments, a construct comprises at least two or more terminators. In some embodiments, a construct comprises at least three or more terminators. In some embodiments, a construct comprises at least four or more terminators. In some embodiments, a construct comprises at least five or more terminators.

In some embodiments, a construct comprises a Cauliflower Mosaic virus 35S terminator (TerCaMV35S). In some embodiments, a construct comprises a 3′ UTR+TerCaMV35S. In some embodiments, a construct comprises an Arabidopsis thaliana Actin 2 terminator (TerAthAct2). In some embodiments, a construct comprises a Solanum lycopersicum Histone H4 terminator (TerSlHisH4). In some embodiments, a construct comprises an Agrobacterium tumefaciens nopaline synthase terminator (TerNos). In some embodiments, a construct comprises an Agrobacterium tumefaciens octopine synthase terminator (TerOcs). In some embodiments, a construct comprises an Agrobacterium tumefaciens mannopine synthase terminator (TerMas). In some embodiments, a construct comprises an Agrobacterium tumefaciens agropine synthase terminator (TerAgs). In some embodiments, a construct comprises a Nicotiana tabacum extensin 3 terminator (NtExt3). In some embodiments, a construct comprises an Arabidopsis thaliana Heat Shock Protein 18.2 terminator (AtHSP18.2). In some embodiments, a construct comprises a Solanum lycopersicum Rubisco small subunit 3C terminator (Ter SlRbcS3C). In some embodiments, a construct comprises an Agrobacterium tumefaciens gene 7 terminator (Ter Atug7). In some embodiments, a construct comprises an Epipremnum aureum rrEaH3Blast2 terminator (Ter 7.1). In some embodiments, a construct comprises a Solanum tuberosum Proteinase inhibitor 2 terminator (Ter StPinII). In some embodiments, a construct comprises a Pisum sativum Rubisco small subunit 3A terminator (Ter Pea3A). In some embodiments, a construct comprises an Arabidopsis thaliana Heat Shock Protein terminator (Ter AtHSP) In some embodiments, a construct comprises a 3′ UTR+TerCaMV35S terminator, a TerOcs terminator, and a TerMas terminator. In some embodiments, a construct comprises a 3′ UTR+TerCaMV35S terminator, at least two TerOcs terminators, a TerMas terminator, and a TerCaMV35S terminator. In some embodiments, a construct comprises a 3′ UTR+TerCaMV35S terminator, a NtExt3 terminator, a AtHSP18.2 terminator, and TerNos terminator.

In some embodiments, a construct comprises one or more signal peptides. In some embodiments, a construct comprises at least one nucleic acid sequence that encodes a signal peptide that is or comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 150. In some embodiments, a construct comprises a nucleic acid sequence is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 148.

In some embodiments, a construct comprises a coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 155. In some embodiments a construct comprises, a first coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 156. In some embodiments a construct comprises, a first coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 158. In some embodiments a construct comprises, a second coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 159. In some embodiments a construct comprises, (i) a first coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 156 and (ii) a second coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 157. In some embodiments a construct comprises, (i) a first coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 158 and (ii) a second coding sequence that is or comprises a nucleic acid sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 159.

In some embodiments, a construct comprises one or more matrix attachment regions (MARs). In some embodiments, a construct comprises at least one MAR. In some embodiments, a construct comprises at least two or more MARs. In some embodiments, a construct comprises at least three or more MARs. In some embodiments, one or more MARs are operatively linked to a coding sequence described herein (e.g., a coding sequence, a first coding sequence, a second coding sequence). In some embodiments, one or more MARs flank a construct described herein. In some embodiments, one or more MARs is or comprise one or more TM2 MARs. In some embodiments, one or more MARs is or comprise one or more Pea MARs. In some embodiments, one or more MARs is or comprise one or more RB7 MARs.

In some embodiments, a construct is or comprise a nucleic acid sequence according to SEQ ID NO: 160. In some embodiments, a construct is or comprise a nucleic acid sequence according to SEQ ID NO: 161. In some embodiments, a construct is or comprise a nucleic acid sequence according to SEQ ID NO: 162. In some embodiments, a construct is or comprise a nucleic acid sequence according to SEQ ID NO: 163.

Among other things, the present disclosure provides an engineered plant. In some embodiments, an engineered plant comprises any one of the constructs disclosed herein. In some embodiments, an engineered plant expresses any one of the constructs disclosed herein in a part of the plant. In some embodiments, a part of a plant is or comprises an organelle. In some embodiments, a part of a plant is or comprises a cell. In some embodiments, a part of a plant is or comprises a tissue. In some embodiments, a part of a plant is or comprises an organ. In some embodiments, a part of a plant is or comprises a chloroplast. In some embodiments, a part of a plant is or comprises a vacuole. In some embodiments, a part of a plant is or comprises an epidermal cell. In some embodiments, a part of a plant is or comprises a guard cell. In some embodiments, a part of a plant is or comprises a mesophyll cell. In some embodiments, a part of a plant is or comprises a hypodermal cell. In some embodiments, a part of a plant is or comprises a sub-epidermal cell. In some embodiments, a part of a plant is or comprises dermal tissue. In some embodiments, a part of a plant is or comprises a leaf. In some embodiments, a part of a plant is or comprises a node. In some embodiments, a part of a plant is or comprises a stem. In some embodiments, a part of a plant is or comprises a root.

In some embodiments, an engineered plant produces a pigment. In some embodiments, at least one part of the engineered plant produces a pigment. In some embodiments, at least one part of the engineered plant expresses a pigment. In some embodiments, production of a pigment is attributable to an expression of any one of the construct disclosed herein. In some embodiments, an engineered plant expresses a pigment. In some embodiments, an engineered plant expresses any one of the constructs disclosed herein. In some embodiments, expression of the pigment is attributable to an expression of any one of the construct disclosed herein. In some embodiments, a pigment is or comprises betalain. In some embodiments, a pigment is or comprises betacyanin. In some embodiments, a pigment is or comprises betaxanthins. In some embodiments, a pigment is or comprises betanin. In some embodiments, a part of a plant is or comprises an organelle. In some embodiments, a part of a plant is or comprises a cell. In some embodiments, a part of a plant is or comprises a tissue. In some embodiments, a part of a plant is or comprises an organ. In some embodiments, a part of a plant is or comprises a chloroplast. In some embodiments, a part of a plant is or comprises a vacuole. In some embodiments, a part of a plant is or comprises an epidermal cell. In some embodiments, a part of a plant is or comprises a guard cell. In some embodiments, a part of a plant is or comprises a mesophyll cell. In some embodiments, a part of a plant is or comprises a hypodermal cell. In some embodiments, a part of a plant is or comprises a sub-epidermal cell. In some embodiments, a part of a plant is or comprises dermal tissue. In some embodiments, a part of a plant is or comprises a leaf. In some embodiments, a part of a plant is or comprises a node. In some embodiments, a part of a plant is or comprises a stem. In some embodiments, a part of a plant is or comprises a root.

Among other things, an engineered plant described herein comprises a pigmentation pattern. In some embodiments, at least one part of an engineered plant described herein comprises a pigmentation pattern. In some embodiments, a pigmentation pattern is attributable to an expression of any construct disclosed herein. In some embodiments, a pigmentation pattern comprises a color. In some embodiments, a color is or comprises a pink color. In some embodiments, a color is or comprises a red color. In some embodiments, a color is or comprises a purple color. In some embodiments, a color is or comprises a brown color. In some embodiments, a color is or comprises a pink color variant. In some embodiments, a color is or comprises a red color variant. In some embodiments, a color is or comprises a purple color variant. In some embodiments, a color is or comprises a brown color variant.

In some embodiments, a color is or comprises a light red pink color. In some embodiments, a color is or comprises a medium red pink color. In some embodiments, a color is or comprises a pink color. In some embodiments, a color is or comprises a light blue pink color. In some embodiments, a color is or comprises a medium blue pink color. In some embodiments, a color is or comprises a dark blue pink color. In some embodiments, a color is or comprises an orange red color. In some embodiments, a color is or comprises a light red color. In some embodiments, a color is or comprises a medium red color. In some embodiments, a color is or comprises a dark red color. In some embodiments, a color is or comprises a medium purple red color. In some embodiments, a color is or comprises a dark purple red color. In some embodiments, a color is or comprises a brown red color. In some embodiments, a color is or comprises a medium brown purple color. In some embodiments, a color is or comprises a dark brown purple color. In some embodiments, a color is or comprises a medium purple color. In some embodiments, a color is or comprises a dark purple color. In some embodiments, a color is or comprises a light violet color. In some embodiments, a color is or comprises a medium violet color. In some embodiments, a color is or comprises a dark violet color. In some embodiments, a color is or comprises a light blue violet color. In some embodiments, a color is or comprises a medium blue violet color. In some embodiments, a color is or comprises a dark blue violet color. In some embodiments, a color is or comprises a light violet blue color. In some embodiments, a color is or comprises a medium violet blue color. In some embodiments, a color is or comprises a dark violet blue color. In some embodiments, a color is or comprises a violet color. In some embodiments, a color is or comprises a crimson color. In some embodiments, a color is or comprises a scarlet color. In some embodiments, a color is or comprises a burgundy color. In some embodiments, a color is or comprises a cherry red color. In some embodiments, a color is or comprises a tomato red color. In some embodiments, a color is or comprises a fire engine red color. In some embodiments, a color is or comprises a ruby color. In some embodiments, a color is or comprises a maroon color. In some embodiments, a color is or comprises a brick red color. In some embodiments, a color is or comprises a raspberry color. In some embodiments, a color is or comprises a vermilion color. In some embodiments, a color is or comprises a cardinal red color. In some embodiments, a color is or comprises a rose red color. In some embodiments, a color is or comprises a blood red color. In some embodiments, a color is or comprises a salmon red color.

In some embodiments, a pigmentation pattern comprises a color pattern. In some embodiments, a color pattern is or comprises a uniform pattern. In some embodiments, a color pattern is or comprises a striped pattern. In some embodiments, a color pattern is or comprises a banded pattern. In some embodiments, a color pattern is or comprises a sectorial pattern. In some embodiments, a color pattern is or comprises a variegated pattern. In some embodiments, a color pattern is or comprises a gradient pattern. In some embodiments, a color pattern is or comprises a non-uniform pattern. In some embodiments, a color pattern is or comprises a mottled pattern. In some embodiments, a color pattern is or comprises a speckled pattern. In some embodiments, a color pattern is or comprises a spotty pattern. In some embodiments, a color pattern is or comprises a patchy pattern. In some embodiments, a color pattern is or comprises an irregular pattern.

In some embodiments, an engineered plant is or comprises an ornamental plant. In some embodiments, an engineered plant is or comprises an indoor ornamental plant. In some embodiments, an engineered plant is of the family Araceae. In some embodiments, an engineered plant is Epipremnum aureum.

Among other things, the present disclosure provides an agrobacterium comprising a construct. In some embodiments, an agrobacterium comprises any construct disclosed herein. In some embodiments, an agrobacterium comprises a construct comprising a nucleic acid sequence according to SEQ ID NO: 160. In some embodiments, an agrobacterium comprises a construct comprising a nucleic acid sequence according to SEQ ID NO: 161. In some embodiments, an agrobacterium comprises a construct comprising a nucleic acid sequence according to SEQ ID NO: 162. In some embodiments, an agrobacterium comprises a construct comprising a nucleic acid sequence according to SEQ ID NO: 163.

Among other things, the present disclosure provides methods. In some embodiments, a method comprises method comprising introducing to a part of a plant any construct disclosed herein. In some embodiments, a method comprises (a) introducing to a part of a plant an agrobacterium disclosed herein, and (b) cultivating or maintaining the part of the plant.

In accordance with various embodiments, the present disclosure provides a method of manufacturing a plant disclosed herein. A method of making, producing, and/or manufacturing an engineered plant disclosed herein. A method of propagating a plant disclosed herein.

A system comprising at least one container comprising at least one cavity suitable for a plant disclosed herein.

### Definitions

The scope of the present disclosure is defined by the claims appended hereto and is not limited by some embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., some embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, as is common practice, polynucleotide or polypeptide sequences are typically presented in 5′ to 3′ or N-terminus to C-terminus order, from left to right unless otherwise indicated.

Allele: As used herein, the term “allele” refers to one of two or more existing genetic variants of a specific polymorphic genomic locus.

Amino acid: In its broadest sense, as used herein, the term “amino acid” refers to a compound and/or substance that can be incorporated into a polypeptide chain, e.g., through formation of one or more peptide bonds. In some embodiments, an amino acid has a general structure, e.g., H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to an amino acid, other than standard amino acids, which in some embodiments may be or have been prepared synthetically and in some embodiments may be or have been obtained from a natural source. In some embodiments, an amino acid, including a carboxy- and/or amino-terminal amino acid in a polypeptide, can contain a structural modification as compared with the general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within +10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, two or more events, conditions, or entities may be described as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” can refer to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element. In some embodiments, a characteristic sequence element is a sequence element that is found in all members of a family of polypeptides or nucleic acids, and therefore can be used by those of ordinary skill in the art to define members of the family.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Conservative: As used herein, the term “conservative” refers to instances describing a conservative amino acid substitution, including a substitution of an amino acid residue by another amino acid residue having a side chain R group with similar structural, chemical (e.g., charge or hydrophobicity), and/or functional properties. In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., Science 256:1443, 1992, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. In some embodiments, a “conservative” substitution is considered a “homologous” residue for purposes of calculating percent homology between amino acid sequences.

EXEMPLARY CONSERVATIVE AMINO
ACID SUBSTITUTIONS

| For | | |
|---|---|---|
| Amino Acid | Code | Replace With |
| Alanine | A | D-ala, Gly, Aib, β-Ala, Acp, L-Cys, |
| | | D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid |
| | | (Kauer, U.S. Pat. No. 4,511,390) |
| Serine | S | D-Ser, Thr, D-Thr, allo-Thr, Met, D-Met, |
| | | Met (O), D-Met (O), L-Cys, D-Cys |
| Threonine | T | D-Thr, Ser, D-Ser, allo-Thr, Met, D-Met, |
| | | Met (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard or reference against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (e.g., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Engineered: In general, as used herein, the term “engineered” refers to an aspect of having been manipulated by the hand of man. For example, in some embodiments, a cell or organism may be considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity. In some embodiments, a cell or organism may be considered to be “engineered” if it has been handled or cultivated in a manner involving one or more interventions by man.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (bifunctional) or many functions (multifunctional).

Gene: As used herein, the term “gene” refers to a DNA sequence in a chromosome that codes for a gene product (e.g., an RNA product, e.g., a polypeptide product). In some embodiments, a gene includes a coding sequence (e.g., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Heterologous: The term “heterologous”, as used herein to refer to an entity (e.g., a gene or polypeptide) that is present in a different source, in a different arrangement, and/or in a different condition or state from that in which it is presently found. To give but one example, in some embodiments, a gene or polypeptide that is not naturally found in a particular organism is considered to be heterologous to that organism. Alternatively or additionally, in some embodiments, a gene or polypeptide that is not naturally found in a particular cell may be considered to be heterologous to that cell if introduced into it (e.g., via a construct (e.g., a vector)), even if that gene or polypeptide might naturally be found in a different cell of the same type. In some embodiments, a construct (e.g., a vector) may be considered to be heterologous to a cell when it has been introduced into the cell, and/or a copy of a gene included in such construct (e.g., a vector) may be considered to be heterologous to that particular cell even if an endogenous copy of the same gene exists in the cell. Where a plurality of different heterologous polypeptides are to be introduced into and/or expressed by a host cell, different polypeptides may be from different source organisms, or from the same source organism. To give but one example, in some cases, individual polypeptides may represent individual subunits of a complex protein activity and/or may be required to work in concert with other polypeptides in order to achieve the goals of the present invention. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acid sequences encoding the polypeptide that have been adjusted to accommodate codon preferences of the host cell and/or to link the encoding sequences with regulatory elements active in the host cell.

Homology: As used herein, the term “homology” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially homologous” to one another if their sequences are at least 25%, 30%, 35%, 40%, 45%, 50%, 55%, 60%, 65%, 70%, 75%, 80%, 85%, 90%, 95%, or 99% homologous, meaning that identical or homologous residues are present in corresponding positions of both molecules. Calculation of percent homology of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (e.g., first and second) are identical at that position. When a position in the first sequence is occupied by the same residue or by a structurally and/or functionally related residue (as will be understood by those skilled in the art, in context), then the two molecules are considered “homologous” at that position. Percent homology between two sequences is a function of the number of homologous positions shared by the two sequences being compared, taking into account the number of gaps, and the length of each gap, which needs to be introduced for optimal alignment of the two sequences. Comparison of sequences and determination of percent homology between two sequences can be accomplished using a mathematical algorithm. For example, percent homology between two nucleotide sequences can be determined using the algorithm of Meyers and Miller (CABIOS, 1989, 4:11-17, which is herein incorporated by reference in its entirety), which has been incorporated into the ALIGN program (version 2.0).

Host Cell: As used herein, the “host cell” is a cell (e.g., a plant, fungal, or bacterial cell) that is manipulated according to the present invention, e.g., to receive a construct (e.g., a vector). In some instances, the term “modified host cell” may be used to refer to a host cell which has been modified, engineered, or manipulated in accordance with the present invention as compared with a parental cell (which may, in some embodiments, be a naturally occurring parental cell or, in other embodiments, may be a parental cell that itself has been engineered or manipulated, including as a host cell). Persons of skill upon reading this disclosure will understand that such terms typically refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (e.g., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4:11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Isolated: As used herein, the term “isolated”, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, the term “operably linked” refers to a juxtaposition wherein the components described are in a relationship permitting them to function in their intended manner. A control element operably linked to a functional element is associated in such a way that expression and/or activity of the functional element is achieved under conditions compatible with the control element. In some embodiments, operably linked control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pathogenic: Those skilled in the art will appreciate that the term “pathogenic” generally refers to an ability to or character of causing disease. In some embodiments, a particular organism or condition may be characterized as or understood to be pathogenic if its presence under relevant circumstances creates a significant and relevant risk of disease to individual(s) who may be present in and/or exposed to the circumstances. Thus, in some embodiments, as will be understood in the art, “pathogenicity” of a particular organism may be impacted by one or more features or elements of context (e.g., amount of organism, size of space, probability of co-localization of organism and potentially susceptible individual, degree of filtration and/or airflow, etc). Alternatively, in some embodiments, an organism may be considered to be “pathogenic” if a material risk of disease would exist if a potentially susceptible individual were exposed to the organism, e.g., under particular standard or experimental or reference conditions.

Phytosphere: The term “phytosphere” will be understood by those skilled in the art to refer to the ecosystem of a plant (e.g., the interior and/or exterior of a plant). In some embodiments, a phytosphere may be or comprise one or more of a phyllosphere, endosphere, and/or rhizosphere.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Polynucleotide: As used herein, the term “polynucleotide” refers to a polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (e.g., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct (e.g., a vector) transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of an ornamental indoor plant, microbiome component, etc).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to a non-coding region of a nucleic acid (e.g., DNA) that regulates one or more aspects of expression of one or more particular genes. In some embodiments, a regulatory element may act in cis with a gene it regulates. In some embodiments, a regulatory element may act in trans with a gene it regulates. In some embodiments, a regulatory element is apposed to or “in the neighborhood” of a gene that it regulates. In some embodiments, a regulatory element, even if in cis with a gene it regulates, is distinct from the gene. In some embodiments, a regulatory element impairs or enhances transcription of one or more genes. In some embodiments, a regulatory sequence refers to a nucleic acid sequence which regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus, bacterium, fungus), a plant, or an animal (e.g., a human). In some embodiments, a source of interest is or comprises biological tissue or fluid. In some embodiments, a biological fluid may be or comprise an intracellular fluid, an extracellular fluid, an intravascular fluid, an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab, scraping, surgery, washing or lavage. In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Source organism: The term “source organism”, as used herein, refers to the organism in which a particular agent (e.g., a particular nucleic acid, polypeptide, etc.) can be found in nature. Thus, for example, if one or more heterologous polypeptides is/are being expressed in a host organism, the organism in which the polypeptides are expressed in nature (and/or from which their genes were originally cloned) may be referred to as the “source organism”. Where multiple heterologous polypeptides are being expressed in a host organism, one or more source organism(s) may be utilized for independent selection of each of the heterologous polypeptide(s). It will be appreciated that any and all organisms that naturally contain relevant polypeptide sequences may be used as source organisms in accordance with the present invention. In some embodiments, representative source organisms may be or include, for example, one or more of animal (e.g., mammal, reptile, fish, bird, insect, etc), plant, microbial (e.g., fungal (e.g., yeast), algal, bacterial [e.g., cyanobacterial, archaebacterial, etc] protozoal, etc).

Stomatal Flux: As used herein, the term “stomatal flux” refers to the cycling of a stoma opening, from open-to-closed, or closed-to-open. Stomatal flux may also refer to the propensity for the stoma to appear in one state or the other, e.g., open or closed.

Subject: As used herein, the term “subject” refers an organism (e.g., a plant, a microbe, etc). In many embodiments, where a subject is a plant, it may be an indoor plant, e.g., an ornamental indoor plant. In some embodiments, a plant subject may be in seed form. In some embodiments, a plant subject may be a plant callus. In some embodiments, a subject can be manipulated (e.g., engineered), for example to better serve a specific purpose.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome. Such a variant is referred to herein as a loss-of-function variant. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide.

Vector: As used herein, the term “vector” or “construct” or “polynucleotide construct” refers to a nucleic acid capable of carrying (e.g., into a cell) at least one heterologous polynucleotide with which it has been linked. In some embodiments, a vector can be or comprise a plasmid, a transposon, a cosmid, an artificial chromosome (e.g., a human artificial chromosome (HAC), a yeast artificial chromosome (YAC), a bacterial artificial chromosome (BAC), a P1-derived artificial chromosome (PAC)), a viral vector, a Gateway® plasmid, etc. In some embodiments, a vector may include sufficient cis-acting elements for expression; alternatively or additionally, elements for expression can be supplied by a cell or system into which the vector is introduced. In some embodiments, a vector may include one or more genetic elements (e.g., origin of replication, primer binding site, etc.) sufficient to achieve replication of the vector in a relevant cell or system. In some embodiments (e.g., bacterial vectors having a bacterial origin of replication and episomal mammalian vectors), a vector may be capable of autonomous replication in a cell or system into which it is introduced. Other vectors (e.g., non-episomal mammalian vectors) can be into nucleic acid(s) already present in such system (e.g., into the genome of a host cell), so that they are replicated along with such present nucleic acid(s). In some embodiments, a vector may be capable of directing expression of genes they carry; such vectors are referred to herein as “expression vectors.”

Volatile Organic Compound: Those of ordinary skill in the art will appreciate that the term “Volatile Organic Compound” (“VOC”) is typically used to refer to compounds that have relatively high vapor pressure and low water solubility. In some embodiments, a VOC may be a carbon-containing compound, excluding carbon monoxide, carbon dioxide, carbonic acid, metallic carbides or carbonates, and ammonium carbonate, which participates in atmospheric photochemical reactions. In some embodiments, a VOC may be or comprise a human made chemical, for example such as may have been used and/or produced in the manufacture of an entity such as a paint, a varnish, a wax, a pharmaceutical, a refrigerant, a cleaning or disinfecting product, a degreasing product, a fuel, etc. Alternatively or additionally, in some embodiments, a VOC may be or comprise a solvent, e.g., an industrial solvent (e.g., trichloroethylene), a fuel oxygenates (e.g., methyl tert-butyl ether (MTBE)), a by-product produced by chlorination in water treatment (e.g., chloroform), etc. Still further alternatively or additionally, in some embodiments, a VOC may be or comprise a component of a petroleum fuels, a hydraulic fluid, a paint thinner, a dry cleaning agent, etc. VOCs are common ground-water contaminants. In some embodiments, a VOC may be emitted (e.g., as a gas) from a solid or liquid such as, for example, a paint or lacquer, a paint stripper, cleaning supplies, pesticides, building materials or furnishings, office equipment such as copiers and printers, a correction fluid or carbonless copy paper, graphics and/or craft materials including glues and adhesives, permanent markers, photographic solutions, etc. In some embodiments, a VOC has a vapor pressure of about 0.01 kPa or more 20° C., or otherwise having a corresponding volatility under the particular conditions in which it is utilized and/or maintained.

### BRIEF DESCRIPTION OF THE DRAWING

FIG. 1 is an exemplary schematic of a betalain biosynthesis pathway.

FIG. 2 is an exemplary construct comprising betalain biosynthesis enzymes.

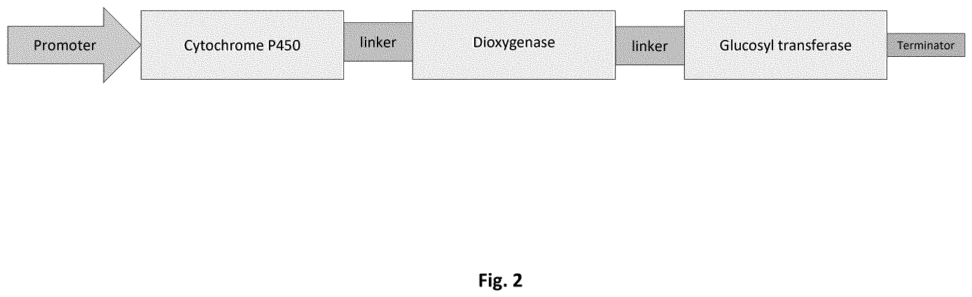

FIG. 3 is a graph showing levels of betanin pigment in exemplary engineered indoor plants. The X axis represents samples collected from plants and the Y axis represents levels of betanin (milligrams of betanin per (fresh) weight of the plant (grams)) as determined by liquid chromatography-mass spectrometry (LC-MS).

FIG. 4 is a picture of an engineered red plant.

FIG. 5 is a graph showing levels of betanin pigment in exemplary engineered indoor plants. The X axis represents samples collected from plants and the Y axis represents levels of betanin (milligrams of betanin per (fresh) weight of the plant (grams)) as determined by LC-MS. Each sample corresponds to an exemplary engineered indoor plant as follows: samples 1 and 2 correspond to plant 1, samples 3 and 4 correspond to plant 2, samples 5 and 6 correspond to plant 3, samples 7 and 8 correspond to plant 4, samples 9 and 10 correspond to plant 5, samples 11 and 12 correspond to plant 6, sample 13 corresponds to plant 7, sample 14 corresponds to plant 8, sample 15 corresponds to plant 9, and sample 16 corresponds to plant 10. Plants 1-8 were transformed with PL-888, and plants 9 and 10 were transformed with PL-1044.

FIG. 6 is a graph showing a ratio of levels of betanin pigment to chlorophyll a pigment in exemplary engineered indoor plants. The X axis represents samples collected from plants and the Y axis represents the ratio of levels of betanin to the ratio of levels of chlorophyll a. Each sample corresponds to an exemplary engineered indoor plant as follows: samples 1 and 2 correspond to plant 1, samples 3 and 4 correspond to plant 2, samples 5 and 6 correspond to plant 3, samples 7 and 8 correspond to plant 4, samples 9 and 10 correspond to plant 5, samples 11 and 12 correspond to plant 6, sample 13 corresponds to plant 7, sample 14 corresponds to plant 8, sample 15 corresponds to plant 9, and sample 16 corresponds to plant 10. Plants 1-8 were transformed with PL-888, and plants 9 and 10 were transformed with PL-1044.

### DETAILED DESCRIPTION OF CERTAIN EMBODIMENTS

### Betalain

Among other things, the present disclosure provides polynucleotides, e.g., polynucleotides comprising a coding sequence that encodes a pigment-producing polypeptide, as well as engineered chlorophyll-containing and/or photosynthetic organisms (CPOS) (e.g., plant) including such polynucleotides and methods including such polynucleotides and and/or CPOS.

Betalains are nitrogenous red and yellow pigments found in a single order of plants, the Caryophyllales, and in some higher order fungi, and there is a limited availability of natural betalain sources (see, e.g., Polturak G, Aharoni A. Advances and future directions in betalain metabolic engineering. New Phytol. 2019 December; 224 (4): 1472-1478., the contents of which is hereby incorporated by reference herein in its entirety).

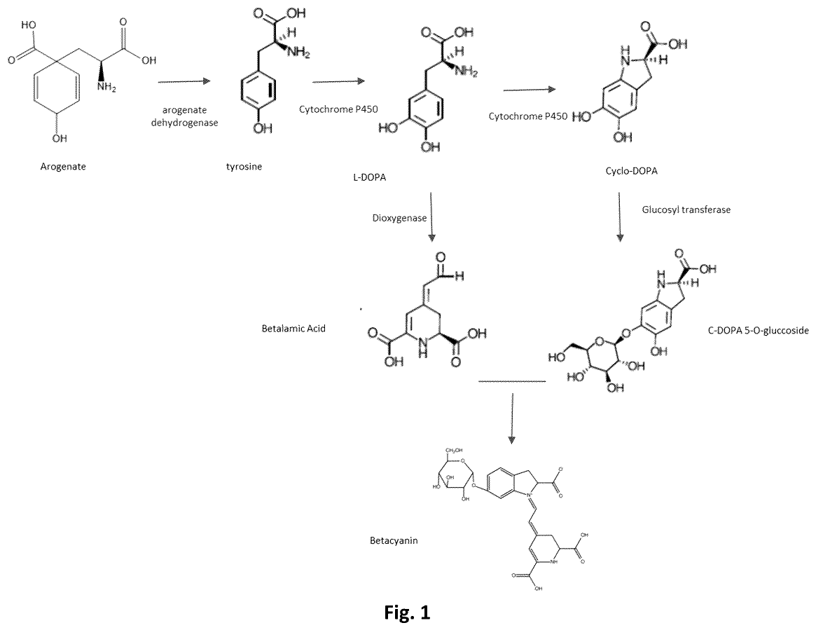

The Betalain biosynthesis pathway is depicted in FIG. 1. There is an initial non-committed step of oxidative decarboxylation of arogenate which is catalyzed by arogenate dehydrogenase (ADH). Tyrosine hydroxylation is catalyzed by a cytochrome P450 polypeptide (CYP76AD1, CYP76AD5, CYP76AD6, and/or CYP76AD15) to form 1-3,4-dihydroxyphenylalanine (L-DOPA). Subsequent processing of L-DOPA generates betacyanin (which has red-violet color) or betaxanthin (which is yellow).

Betacyanin is produced through a conversion of L-DOPA into two different metabolites that then are reacted with each other. Specifically, (1) L-DOPA is converted into betalamic acid through the action of L-DOPA 4,5-dioxygenase (DODA); and (2) L-DOPA is converted into cDOPA 5-O-glucoside in a two-step process that involves cyclation by cytochrome P450 (CYP76AD1 and/or CYP76AD3) to generate cyclo-DOPA, followed by glucosylation by cyclo-DOPA 5-O-glucosyltransferase (cDOPA5GT) to generate the cDOPA 5-O-glucoside. Condensation of the betalamic acid with the cyclo-DOPA 5-O-glucoside forms betacyanin.

In some embodiments, a gene (e.g., a heterologous gene) described herein is or comprises a pigment-producing gene. In some embodiments, a pigment-producing gene is or comprises a gene that encodes a polypeptide, a fragment thereof, or a characteristic portion thereof that directly or indirectly contributes to biosynthesis, modification, accumulation, or stabilization of a pigment in a cell, tissue, and/or organ. In some embodiments, a pigment-producing gene encodes a polypeptide, a fragment thereof, or a characteristic portion thereof (e.g., enzyme) that catalyzes at least one biochemical reaction in a biosynthetic pathway (e.g., a betalain biosynthesis pathway). In some embodiments, a pigment-producing gene encodes a polypeptide, a fragment thereof, or a characteristic portion thereof that comprises an enzyme that produces one or more precursors and/or one or more substrates for at least one biochemical reaction in a biosynthetic pathway (e.g., a betalain biosynthesis pathway). In some embodiments, a pigment-producing gene encodes a polypeptide, a fragment thereof, or a characteristic portion thereof that may be known to be involved in a first biosynthetic pathway (e.g., a betalain biosynthesis pathway) and directly or indirectly contribute to biosynthesis, modification, accumulation, or stabilization of one or more precursors and/or one or more substrates for a second biosynthetic pathway. In some embodiments, the first and second biosynthetic pathway are the same. In some embodiments, the first and second biosynthetic pathway are different. In some embodiments, a pigment-producing gene comprises or is a pigment-producing polynucleotide sequence.

In some embodiments, a polypeptide described herein is or comprise a pigment-producing polypeptide. In some embodiments, a pigment-producing polypeptide is or comprises a polypeptide, a fragment thereof, or a characteristic portion thereof that directly or indirectly contributes to biosynthesis, modification, accumulation, or stabilization of a pigment in a cell, tissue, and/or organ. In some embodiments, a pigment-producing polypeptide is or comprises a polypeptide, a fragment thereof, or a characteristic portion thereof that comprises an enzyme that catalyzes at least one biochemical reaction in a biosynthetic pathway (e.g., a betalain biosynthesis pathway). In some embodiments, a pigment-producing polypeptide is or comprises a polypeptide, a fragment thereof, or a characteristic portion thereof comprises an enzyme that produces one or more precursors and/or one or more substrates for at least one biochemical reaction in a biosynthetic pathway (e.g., a betalain biosynthesis pathway). In some embodiments, a pigment-producing polypeptide is or comprises a polypeptide, a fragment thereof, or a characteristic portion thereof that may be known to be involved in a first biosynthetic pathway and directly or indirectly contribute to biosynthesis, modification, accumulation, or stabilization of one or more precursors and/or one or more substrates for a second biosynthetic pathway. In some embodiments, the first and second biosynthetic pathway are the same. In some embodiments, the first and second biosynthetic pathway are different.

In some embodiments, a pigment-producing gene is or comprises a gene that encodes a polypeptide, a fragment thereof, or a characteristic portion thereof that directly or indirectly contributes to biosynthesis, modification, accumulation, or stabilization of betalain in a cell, tissue, and/or organ. In some embodiments, a pigment-producing gene is or comprises a gene that encodes a polypeptide, a fragment thereof, or a characteristic portion thereof that directly or indirectly contributes to biosynthesis, modification, accumulation, or stabilization of betacyanin in a cell, tissue, and/or organ. In some embodiments, a pigment-producing gene is or comprises a gene that encodes a polypeptide, a fragment thereof, or a characteristic portion thereof that directly or indirectly contributes to biosynthesis, modification, accumulation, or stabilization of betaxanthin in a cell, tissue, and/or organ. In some embodiments, a pigment-producing gene is or comprises a gene that encodes a polypeptide, a fragment thereof, or a characteristic portion thereof that directly or indirectly contributes to biosynthesis, modification, accumulation, or stabilization of betanin in a cell, tissue, and/or organ.

In some embodiments, a pigment-producing polypeptide is or comprises a polypeptide, a fragment thereof, or a characteristic portion thereof that directly or indirectly contributes to biosynthesis, modification, accumulation, or stabilization of betalain in a cell, tissue, and/or organ. In some embodiments, a pigment-producing polypeptide is or comprises a polypeptide, a fragment thereof, or a characteristic portion thereof that directly or indirectly contributes to biosynthesis, modification, accumulation, or stabilization of betacyanin in a cell, tissue, and/or organ. In some embodiments, a pigment-producing polypeptide is or comprises a polypeptide, a fragment thereof, or a characteristic portion thereof that directly or indirectly contributes to biosynthesis, modification, accumulation, or stabilization of betaxanthin in a cell, tissue, and/or organ. In some embodiments, a pigment-producing polypeptide is or comprises a polypeptide, a fragment thereof, or a characteristic portion thereof that directly or indirectly contributes to biosynthesis, modification, accumulation, or stabilization of betanin in a cell, tissue, and/or organ.

### Pigment-Producing Polynucleotide Sequences

Among other things, the present disclosure provides pigment-producing genes encoding pigment-producing polypeptides. In some embodiments, a pigment-producing gene may refer to a betalain-related gene or a betalain biosynthesis pathway gene.

In some embodiments, a pigment-producing gene encodes a cytochrome P450 polypeptide. In some embodiments, a pigment-producing gene encodes a CYP76AD1 polypeptide, a CYP76AD5 polypeptide, a CYP76AD6 polypeptide, or a CYP76AD15 polypeptide. In some embodiments, a pigment-producing gene encodes a CYP76AD1 polypeptide.

In some embodiments, a pigment-producing gene encodes a DODA polypeptide. In some embodiments, a pigment-producing gene encodes a DOPA5GT polypeptide. In some embodiments, a pigment-producing gene encodes a cDOPA5GT polypeptide.

In some embodiments, a pigment-producing gene encodes an arogenate dehydrogenase (ADH) polypeptide. In some embodiments, a pigment-producing gene encodes an ADHα polypeptide.

In some embodiments, a pigment-producing gene encodes a 3-deoxy-D-arabino-heptulosonate 7-phosphate synthase (DHAP synthase) polypeptide. In some embodiments, a pigment-producing gene encodes a DHAP synthase comprising an L175Q mutation (AroG^{L175Q}).

In some embodiments, a pigment-producing gene is at least 85%, at least 90%, at least 91%, at least 92%, at least 93%, at least 94%, at least 95%, at least 96%, at least 97%, at least 98%, or at least 99% identical to a sequence included in Table 1. In some embodiments, a pigment-producing gene described herein has a polynucleotide sequence provided in Table 1.

TABLE 1
DNA Sequences of Exemplary Betalain-Related Polynucleotides

| Description | Sequence | SEQ ID |
|---|---|---|
| | | NO: |
| CYP76AD1 | AATGGATCATGCGACCCTCGCCATGATCCTCGCGATCTGGTTCATCAG | 1 |
| | CTTCCACTTCATCAAGCTGCTGTTCTCCCAGCAGACCACCAAGCTGCT | |
| | TCCGCCAGGACCAAAGCCGCTTCCGATCATCGGCAACATCCTTGAGGT | |
| | GGGCAAGAAGCCGCATCGGTCCTTCGCCAACCTCGCCAAGATTCACGG | |
| | CCCACTCATTTCCCTCAGACTCGGCTCTGTGACCACCATCGTTGTGTC | |
| | CTCTGCCGACGTGGCCAAAGAGATGTTCCTCAAGAAGGATCACCCGCT | |
| | CTCCAACCGCACGATCCCGAATAGTGTTACAGCCGGCGACCACCACAA | |
| | GCTCACCATGTCTTGGCTCCCGGTGTCTCCGAAGTGGCGCAACTTCCG | |
| | CAAGATTACCGCCGTGCATCTGCTCTCCCCACAGAGACTCGATGCCTG | |
| | CCAGACATTCAGGCACGCCAAGGTGCAGCAGCTCTACGAGTACGTTCA | |
| | AGAGTGCGCCCAGAAAGGCCAGGCCGTGGATATTGGCAAGGCCGCCTT | |
| | TACGACCAGCCTCAACCTCCTCAGCAAGCTGTTCTTCAGCGTCGAGCT | |
| | GGCGCACCACAAGTCCCATACCAGCCAAGAGTTCAAAGAGCTGATCTG | |
| | GAACATCATGGAAGATATAGGCAAGCCGAACTACGCCGACTACTTCCC | |
| | GATTCTCGGCTGCGTTGACCCATCTGGCATTAGAAGAAGGCTCGCCTG | |
| | CTCCTTCGACAAGCTGATCGCCGTGTTCCAGGGCATCATCTGCGAGAG | |
| | ACTCGCCCCAGATTCCTCCACCACAACTACCACCACCACCGACGACGT | |
| | GCTCGATGTGCTCCTCCAGCTGTTCAAGCAGAACGAGCTGACGATGGG | |
| | CGAGATCAACCACCTCCTCGTGGACATCTTCGACGCCGGCACCGATAC | |
| | CACATCCTCCACATTCGAGTGGGTGATGACCGAGCTGATCCGCAATCC | |
| | AGAGATGATGGAAAAGGCCCAAGAGGAAATCAAGCAGGTCCTCGGCAA | |
| | GGACAAGCAGATCCAAGAGTCCGACATCATCAACCTGCCGTACCTCCA | |
| | GGCGATCATCAAAGAGACACTCCGCCTCCATCCGCCGACCGTGTTCTT | |
| | GCTCCCAAGAAAGGCCGACACCGATGTCGAGCTGTACGGCTACATCGT | |
| | GCCGAAGGATGCCCAGATCCTCGTGAACCTCTGGGCCATTGGCAGGGA | |
| | CCCAAACGCCTGGCAGAACGCCGATATTTTCAGCCCAGAGCGCTTCAT | |
| | CGGCTGCGAGATCGATGTTAAGGGCCGCGATTTCGGCCTCCTTCCATT | |
| | TGGCGCTGGCCGCAGAATTTGCCCAGGCATGAATCTCGCCATCAGGAT | |
| | GCTCACCCTCATGCTCGCCACACTCCTCCAGTTCTTCAACTGGAAGCT | |
| | CGAAGGCGACATCTCCCCGAAGGACCTCGACATGGACGAGAAGTTCGG | |
| | CATTGCGCTCCAAAAGACCAAGCCGCTCAAGCTCA | |
| CYP76AD1 | ATGGATCATGCGACCCTCGCCATGATCCTCGCGATCTGGTTCATCAGC | 127 |
| (2) | TTCCACTTCATCAAGCTGCTGTTCTCCCAGCAGACCACCAAGCTGCTT | |
| | CCGCCAGGACCAAAGCCGCTTCCGATCATCGGCAACATCCTTGAGGTG | |
| | GGCAAGAAGCCGCATCGGTCCTTCGCCAACCTCGCCAAGATTCACGGC | |
| | CCACTCATTTCCCTCAGACTCGGCTCTGTGACCACCATCGTTGTGTCC | |
| | TCTGCCGACGTGGCCAAAGAGATGTTCCTCAAGAAGGATCACCCGCTC | |
| | TCCAACCGCACGATCCCGAATAGTGTTACAGCCGGCGACCACCACAAG | |
| | CTCACCATGTCTTGGCTCCCGGTGTCTCCGAAGTGGCGCAACTTCCGC | |
| | AAGATTACCGCCGTGCATCTGCTCTCCCCACAGAGACTCGATGCCTGC | |
| | CAGACATTCAGGCACGCCAAGGTGCAGCAGCTCTACGAGTACGTTCAA | |
| | GAGTGCGCCCAGAAAGGCCAGGCCGTGGATATTGGCAAGGCCGCCTTT | |
| | ACGACCAGCCTCAACCTCCTCAGCAAGCTGTTCTTCAGCGTCGAGCTG | |
| | GCGCACCACAAGTCCCATACCAGCCAAGAGTTCAAAGAGCTGATCTGG | |
| | AACATCATGGAAGATATAGGCAAGCCGAACTACGCCGACTACTTCCCG | |
| | ATTCTCGGCTGCGTTGACCCATCTGGCATTAGAAGAAGGCTCGCCTGC | |
| | TCCTTCGACAAGCTGATCGCCGTGTTCCAGGGCATCATCTGCGAGAGA | |
| | CTCGCCCCAGATTCCTCCACCACAACTACCACCACCACCGACGACGTG | |
| | CTCGATGTGCTCCTCCAGCTGTTCAAGCAGAACGAGCTGACGATGGGC | |
| | GAGATCAACCACCTCCTCGTGGACATCTTCGACGCCGGCACCGATACC | |
| | ACATCCTCCACATTCGAGTGGGTGATGACCGAGCTGATCCGCAATCCA | |
| | GAGATGATGGAAAAGGCCCAAGAGGAAATCAAGCAGGTCCTCGGCAAG | |
| | GACAAGCAGATCCAAGAGTCCGACATCATCAACCTGCCGTACCTCCAG | |
| | GCGATCATCAAAGAGACACTCCGCCTCCATCCGCCGACCGTGTTCTTG | |
| | CTCCCAAGAAAGGCCGACACCGATGTCGAGCTGTACGGCTACATCGTG | |
| | CCGAAGGATGCCCAGATCCTCGTGAACCTCTGGGCCATTGGCAGGGAC | |
| | CCAAACGCCTGGCAGAACGCCGATATTTTCAGCCCAGAGCGCTTCATC | |
| | GGCTGCGAGATCGATGTTAAGGGCCGCGATTTCGGCCTCCTTCCATTT | |
| | GGCGCTGGCCGCAGAATTTGCCCAGGCATGAATCTCGCCATCAGGATG | |
| | CTCACCCTCATGCTCGCCACACTCCTCCAGTTCTTCAACTGGAAGCTC | |
| | GAAGGCGACATCTCCCCGAAGGACCTCGACATGGACGAGAAGTTCGGC | |
| | ATTGCGCTCCAAAAGACCAAGCCGCTCAAGCTCATCCCGATTCCGCGC | |
| | TAC | |
| 4,5DOPA | ATGAAGATGATGAACGGCGAGGACGCCAACGACCAGATGATCAAAGAG | 2 |
| dioxygenase | TCCTTCTTCATCACCCACGGCAACCCGATCCTCACCGTCGAGGATACA | |
| | CATCCGCTCAGGCCGTTCTTCGAGACATGGCGCGAGAAGATTTTCTCC | |
| | AAGAAGCCGAAGGCCATCCTCATCATCTCCGGCCACTGGGAGACAGTG | |
| | AAGCCAACCGTGAACGCCGTGCACATCAACGACACCATCCACGACTTC | |
| | GACGACTACCCAGCCGCCATGTACCAGTTCAAGTACCCAGCTCCAGGC | |
| | GAGCCAGAGCTTGCGAGAAAGGTGGAAGAGATCCTCAAGAAGTCCGGG | |
| | TTCGAGACAGCCGAGACAGACCAAAAGAGGGGCCTTGATCACGGCGCC | |
| | TGGGTTCCACTCATGCTCATGTATCCAGAGGCGGACATCCCGGTGTGC | |
| | CAGCTCTCAGTTCAGCCACATCTCGACGGCACCTACCACTACAATCTC | |
| | GGCAGAGCCCTCGCGCCGCTCAAGAATGATGGCGTGCTCATTATTGGC | |
| | TCCGGCAGCGCCACACATCCACTCGATGAGACACCGCACTACTTCGAT | |
| | GGTGTTGCCCCTTGGGCCGCTGCCTTCGATTCTTGGCTTAGGAAGGCC | |
| | CTCATCAACGGCCGCTTCGAGGAAGTGAACATCTACGAGAGCAAGGCC | |
| | CCGAACTGGAAGCTCGCCCATCCATTTCCAGAGCACTTCTACCCGCTC | |
| | CACGTTGTGCTCGGCGCTGCTGGTGAAAAGTGGAAGGCCGAGCTGATC | |
| | CACTCCTCCTGGGATCATGGCACACTTTGCCACGGCTCCTACAAGTTC | |
| | ACCTCCGCC | |
| CycloDopa5 | CGAGGGCGAGACACAGCACATCCTCATGATCCCGTTCATGGCGCAGGG | 3 |
| -O- | CCACCTCAGGCCATTTCTCGAACTCGCCATGTTCCTCTACAAGCGCTC | |
| glucosyltransferase | CCACGTGATCATCACCCTGCTCACAACTCCGCTCAATGCCGGCTTCCT | |
| (cDOPA5GT) | CAGGCACCTCCTTCACCACCATTCCTACTCCTCCAGCGGCATCAGGAT | |
| | CGTCGAGCTGCCATTCAACTCCACCAACCACGGACTCCCACCGGGCAT | |
| | CGAGAACACCGATAAGCTCACACTCCCGCTCGTGGTGTCCCTCTTCCA | |
| | TTCCACCATCAGCCTCGATCCGCACCTCCGCGATTACATCTCCAGGCA | |
| | TTTCAGCCCAGCCAGGCCACCACTCTGCGTGATCCATGATGTGTTCCT | |
| | CGGCTGGGTTGACCAGGTGGCCAAGGATGTGGGCTCTACAGGCGTGGT | |
| | GTTCACAACAGGCGGCGCTTATGGCACATCCGCCTACGTGTCCATCTG | |
| | GAACGATCTCCCGCACCAGAACTACTCCGACGACCAAGAGTTCCCGCT | |
| | GCCAGGCTTCCCAGAGAACCATAAGTTCCGCAGGTCCCAGCTCCATCG | |
| | GTTCCTCAGATATGCCGACGGCTCCGACGATTGGTCCAAGTATTTCCA | |
| | GCCGCAGCTCCGCCAGTCCATGAAGTCTTTTGGCTGGCTCTGCAACTC | |
| | CGTGGAAGAGATCGAGACACTCGGCTTCTCCATCCTCCGCAACTACAC | |
| | CAAGCTGCCGATCTGGGGCATCGGCCCACTTATTGCTTCCCCAGTGCA | |
| | GCACTCCTCCTCCGACAACAATTCAACAGGCGCCGAGTTCGTGCAGTG | |
| | GCTCAGCCTCAAAGAGCCGGACTCCGTCCTCTACATCTCCTTCGGCTC | |
| | CCAGAACACGATCAGCCCGACGCAGATGATGGAACTCGCTGCTGGCCT | |
| | TGAGTCCTCCGAGAAGCCATTCCTCTGGGTGATCAGAGCCCCGTTCGG | |
| | CTTCGACATCAACGAAGAGATGCGCCCAGAGTGGCTGCCAGAGGGCTT | |
| | TGAGGAACGCATGAAGGTGAAGAAACAGGGCAAGCTCGTGTACAAGCT | |
| | CGGCCCGCAGCTTGAGATCCTCAACCATGAATCCATCGGCGGCTTTCT | |
| | CACCCACTGCGGATGGAACAGCATCCTTGAGTCTCTTCGCGAGGGCGT | |
| | TCCGATGCTTGGATGGCCACTTGCTGCCGAGCAGGCCTACAACCTCAA | |
| | GTACCTCGAAGATGAGATGGGCGTCGCGGTTGAGCTTGCTAGAGGCCT | |
| | CGAAGGCGAGATCTCCAAAGAGAAGGTCAAGCGCATCGTCGAGATGAT | |
| | CCTTGAGCGCAACGAGGGCTCCAAAGGCTGGGAGATGAAGAATCGCGC | |
| | CGTGGAAATGGGCAAAAAGCTCAAGGACGCCGTGAACGAGGAAAAAGA | |
| | GCTGAAGGGCTCCTCCGTGAAGGCGATCGACGATTTCCTCGACGCCGT | |
| | CATGCAGGCCAAACTTGAGCCAAGCCTCCAGTGATAGTGAGCTTCTC | |
| CycloDopa5 | ATGACCGCCATCAAGATGAACACCAACGGCGAGGGCGAGACACAGCAC | 128 |
| -O- | ATCCTCATGATCCCGTTCATGGCGCAGGGCCACCTCAGGCCATTTCTC | |
| glucosyltransferase | GAACTCGCCATGTTCCTCTACAAGCGCTCCCACGTGATCATCACCCTG | |
| (cDOPA5GT) | CTCACAACTCCGCTCAATGCCGGCTTCCTCAGGCACCTCCTTCACCAC | |
| | CATTCCTACTCCTCCAGCGGCATCAGGATCGTCGAGCTGCCATTCAAC | |
| | TCCACCAACCACGGACTCCCACCGGGCATCGAGAACACCGATAAGCTC | |
| | ACACTCCCGCTCGTGGTGTCCCTCTTCCATTCCACCATCAGCCTCGAT | |
| | CCGCACCTCCGCGATTACATCTCCAGGCATTTCAGCCCAGCCAGGCCA | |
| | CCACTCTGCGTGATCCATGATGTGTTCCTCGGCTGGGTTGACCAGGTG | |
| | GCCAAGGATGTGGGCTCTACAGGCGTGGTGTTCACAACAGGCGGCGCT | |
| | TATGGCACATCCGCCTACGTGTCCATCTGGAACGATCTCCCGCACCAG | |
| | AACTACTCCGACGACCAAGAGTTCCCGCTGCCAGGCTTCCCAGAGAAC | |
| | CATAAGTTCCGCAGGTCCCAGCTCCATCGGTTCCTCAGATATGCCGAC | |
| | GGCTCCGACGATTGGTCCAAGTATTTCCAGCCGCAGCTCCGCCAGTCC | |
| | ATGAAGTCTTTTGGCTGGCTCTGCAACTCCGTGGAAGAGATCGAGACA | |
| | CTCGGCTTCTCCATCCTCCGCAACTACACCAAGCTGCCGATCTGGGGC | |
| | ATCGGCCCACTTATTGCTTCCCCAGTGCAGCACTCCTCCTCCGACAAC | |
| | AATTCAACAGGCGCCGAGTTCGTGCAGTGGCTCAGCCTCAAAGAGCCG | |
| | GACTCCGTCCTCTACATCTCCTTCGGCTCCCAGAACACGATCAGCCCG | |
| | ACGCAGATGATGGAACTCGCTGCTGGCCTTGAGTCCTCCGAGAAGCCA | |
| | TTCCTCTGGGTGATCAGAGCCCCGTTCGGCTTCGACATCAACGAAGAG | |
| | ATGCGCCCAGAGTGGCTGCCAGAGGGCTTTGAGGAACGCATGAAGGTG | |
| | AAGAAACAGGGCAAGCTCGTGTACAAGCTCGGCCCGCAGCTTGAGATC | |
| | CTCAACCATGAATCCATCGGCGGCTTTCTCACCCACTGCGGATGGAAC | |
| | AGCATCCTTGAGTCTCTTCGCGAGGGCGTTCCGATGCTTGGATGGCCA | |
| | CTTGCTGCCGAGCAGGCCTACAACCTCAAGTACCTCGAAGATGAGATG | |
| | GGCGTCGCGGTTGAGCTTGCTAGAGGCCTCGAAGGCGAGATCTCCAAA | |
| | GAGAAGGTCAAGCGCATCGTCGAGATGATCCTTGAGCGCAACGAGGGC | |
| | TCCAAAGGCTGGGAGATGAAGAATCGCGCCGTGGAAATGGGCAAAAAG | |
| | CTCAAGGACGCCGTGAACGAGGAAAAAGAGCTGAAGGGCTCCTCCGTG | |
| | AAGGCGATCGACGATTTCCTCGACGCCGTCATGCAGGCCAAACTTGAG | |
| | CCAAGCCTCCAGTGA | |
| ADHa | ATGATATCACTATCCTCTTTTCATCCATCAAGCACAACTGCTACCGCT | 4 |
| | ACCGCAGCTGCCGCAACCACACACCCTCCACAGCAATGCCCCGCATTC | |
| | TCTTCACCACCTTCGCATCTATCCTTGCCGCTACGACACCCTCGTCAA | |
| | CATCTTGTGGTTAGGTGTGGAGGGGGTGGCTCTGCATCGGAGTCCGTA | |
| | TTCAACAGAGATTCAGCCGCAACTAGGGTTTCAAACGATCACTTGGAT | |
| | GTATCTAAAAGGGATGTAAAACTCAAAATTGCTATCATAGGGTTCGGA | |
| | AACTTTGGGCAATTTCTCGCAAAGACTATGGCAAAACAAGGTCATAGA | |
| | GTACTAGCATATTCTCGATCAGACTATTCCCGGGCGGCAAAAGAGATT | |
| | GGAGTTGAATATTTTACTGATGCAGATGACTTGTGTGAGGAACATCCT | |
| | GAAGTAATTCTTTTGTGTACATCTATACTGTCTACTGAGAAGGTGTTG | |
| | CGGTCATTACCTCTCCATAGATTGCGCAGATCAACCCTGTTCGCTGAT | |
| | GTTTTGAGTGTGAAAGAGTTCCCTCGCTCTTTATTTCTGCAACTCTTG | |
| | CCCAAAGATTTTGATATACTCTGTACCCACCCTATGTTTGGGCCAGAC | |
| | AGCGGGAAAGACGGGTGGGGCGGACTCCCATTCGTATTTGACAAAGTA | |
| | AGGGTGGGCAGCGACCAATCTCGGACGTCAAGAGCTGAGGCATTTTTA | |
| | GATGTTTTTAGAAACGCCGGGTGTAGAATGGTGGAAATGTCATGCGTA | |
| | GATCATGACAAACATGCCGCAGGCTCACAATTTATTACACACATGATG | |
| | GGAAGGGTACTGGAAAAACTAGCACTTGAAAATACGCCAATTAACACA | |
| | AAGGGATACGAATCATTACTAAATCTAGTTGATAACACAGCACGCGAC | |
| | AGCTTTGAATTGTTTTACGGCCTATTCCTTTATAACAAGAATGCTATG | |
| | GAGCAGTTGGATAGAATGGACTGGGCTTTTGAGATGGTGAAAAAGCAG | |
| | CTAAGCGGTTATTTGCATGATCTGGTGAGGAAACAACTAATGCTGGAA | |
| | GGGAACAATGATCAGGCGGAGGTCACCTTCGATAAGCCCCTGATGCTA | |
| | CCCTCTCCCACAATTAACCCACCTCAAATAGTGCCCTCCGCTGACATG | |
| | GCTGAGAAAAAGCACGATCTTGTAGTTGTCAATGGGACTAGGTAG | |
| ADHa (2) | ATGATATCACTATCCTCTTTTCATCCATCAAGCACAACTGCTACCGCT | 129 |
| | ACCGCAGCTGCCGCAACCACACACCCTCCACAGCAATGCCCCGCATTC | |
| | TCTTCACCACCTTCGCATCTATCCTTGCCGCTACGACACCCTCGTCAA | |
| | CATCTTGTGGTTAGGTGTGGAGGGGGTGGCTCTGCATCGGAGTCCGTA | |
| | TTCAACAGAGATTCAGCCGCAACTAGGGTTTCAAACGATCACTTGGAT | |
| | GTATCTAAAAGGGATGTAAAACTCAAAATTGCTATCATAGGGTTCGGA | |
| | AACTTTGGGCAATTTCTCGCAAAGACTATGGCAAAACAAGGTCATAGA | |
| | GTACTAGCATATTCTCGATCAGACTATTCCCGGGCGGCAAAAGAGATT | |
| | GGAGTTGAATATTTTACTGATGCAGATGACTTGTGTGAGGAACATCCT | |
| | GAAGTAATTCTTTTGTGTACATCTATACTGTCTACTGAGAAGGTGTTG | |
| | CGGTCATTACCTCTCCATAGATTGCGCAGATCAACCCTGTTCGCTGAT | |
| | GTTTTGAGTGTGAAAGAGTTCCCTCGCTCTTTATTTCTGCAACTCTTG | |
| | CCCAAAGATTTTGATATACTCTGTACCCACCCTATGTTTGGGCCAGAC | |
| | AGCGGGAAAGACGGGTGGGGCGGACTCCCATTCGTATTTGACAAAGTA | |
| | AGGGTGGGCAGCGACCAATCTCGGACGTCAAGAGCTGAGGCATTTTTA | |
| | GATGTTTTTAGAAACGCCGGGTGTAGAATGGTGGAAATGTCATGCGTA | |
| | GATCATGACAAACATGCCGCAGGCTCACAATTTATTACACACATGATG | |
| | GGAAGGGTACTGGAAAAACTAGCACTTGAAAATACGCCAATTAACACA | |
| | AAGGGATACGAATCATTACTAAATCTAGTTGATAACACAGCACGCGAC | |
| | AGCTTTGAATTGTTTTACGGCCTATTCCTTTATAACAAGAATGCTATG | |
| | GAGCAGTTGGATAGAATGGACTGGGCTTTTGAGATGGTGAAAAAGCAG | |
| | CTAAGCGGTTATTTGCATGATCTGGTGAGGAAACAACTAATGCTGGAA | |
| | GGGAACAATGATCAGGCGGAGGTCACCTTCGATAAGCCCCTGATGCTA | |
| | CCCTCTCCCACAATTAACCCACCTCAAATAGTGCCCTCCGCTGACATG | |
| | GCTGAGAAAAAGCACGATCTTGTAGTTGTCAATGGGACTAGG | |
| DODA | ATGAAAATGATGAATGGTGAGGACGCCAACGATCAAATGATTAAAGAG | 5 |
| | TCGTTCTTTATCACGCATGGAAATCCAATTCTCACGGTAGAGGACACA | |
| | CACCCTCTGCGTCCATTCTTTGAAACTTGGAGAGAAAAGATATTTAGT | |
| | AAGAAACCCAAAGCTATTCTGATCATTAGTGGGCATTGGGAAACCGTT | |
| | AAGCCTACAGTGAATGCAGTTCATATCAATGACACCATTCATGATTTT | |
| | GACGATTACCCCGCTGCCATGTATCAATTTAAATACCCTGCTCCCGGA | |
| | GCACCTGAGCTTGCCAGGAAAGTGGAGGAAATTTTGAAAAAGTCCGGC | |
| | TTTGAGACGGCCGAAACCGATGAGAAAAGAGGTCTTGATCATGGCGCG | |
| | TGGGTCCCCCTTATGCTCATGTACCCTGAAGCCGATATACCAGTGTGC | |
| | CAATTAAGTGTTCAGCCTCACCTTGATGGAACCTATCACTATAACCTT | |
| | GGTAGAGCCCTTGCGCCCTTGAAAAACGATGGGGTCTTAATTATAGGG | |
| | AGTGGGAGCGCAACGCACCCCTTGGATGAGACTCCTCATTATTTTGAT | |
| | GGCGTTGCTCCTTGGGCCGCAGCTTTTGACTCTTGGTTACGCAAGGCC | |
| | TTGATTAATGGGCGGTTCGAAGAGGTGAACATATATGAAACTAAAGCT | |
| | CCAAACTGGAAATTGGCTCACCCCTTTCCTGAGCACTTTTATCCCCTC | |
| | CATGTAGTGCTGGGTGCTGCGGGGGAGAAGTGGAAAGCTGAGCTTATA | |
| | CATTCCTCATGGGACCACGGCACCTTGTGCCACGGTTCATACAAATTC | |
| | ACAAGCGCTTAA | |
| DODA (2) | ATGAAAATGATGAATGGTGAGGACGCCAACGATCAAATGATTAAAGAG | 130 |
| | TCGTTCTTTATCACGCATGGAAATCCAATTCTCACGGTAGAGGACACA | |
| | CACCCTCTGCGTCCATTCTTTGAAACTTGGAGAGAAAAGATATTTAGT | |
| | AAGAAACCCAAAGCTATTCTGATCATTAGTGGGCATTGGGAAACCGTT | |
| | AAGCCTACAGTGAATGCAGTTCATATCAATGACACCATTCATGATTTT | |
| | GACGATTACCCCGCTGCCATGTATCAATTTAAATACCCTGCTCCCGGA | |
| | GCACCTGAGCTTGCCAGGAAAGTGGAGGAAATTTTGAAAAAGTCCGGC | |
| | TTTGAGACGGCCGAAACCGATGAGAAAAGAGGTCTTGATCATGGCGCG | |
| | TGGGTCCCCCTTATGCTCATGTACCCTGAAGCCGATATACCAGTGTGC | |
| | CAATTAAGTGTTCAGCCTCACCTTGATGGAACCTATCACTATAACCTT | |
| | GGTAGAGCCCTTGCGCCCTTGAAAAACGATGGGGTCTTAATTATAGGG | |
| | AGTGGGAGCGCAACGCACCCCTTGGATGAGACTCCTCATTATTTTGAT | |
| | GGCGTTGCTCCTTGGGCCGCAGCTTTTGACTCTTGGTTACGCAAGGCC | |
| | TTGATTAATGGGCGGTTCGAAGAGGTGAACATATATGAAACTAAAGCT | |
| | CCAAACTGGAAATTGGCTCACCCCTTTCCTGAGCACTTTTATCCCCTC | |
| | CATGTAGTGCTGGGTGCTGCGGGGGAGAAGTGGAAAGCTGAGCTTATA | |
| | CATTCCTCATGGGACCACGGCACCTTGTGCCACGGTTCATACAAATTC | |
| | ACAAGCGCT | |
| CYP76AD1 | ATGGACCATGCGACCCTTGCAATGATACTAGCCATTTGGTTCATCAGT | 6 |
| (3) | TTTCACTTCATTAAACTACTCTTTAGCCAGCAAACCACTAAACTCCTA | |
| | CCACCGGGTCCCAAACCATTGCCAATCATAGGAAACATCCTTGAGGTG | |
| | GGAAAGAAACCACACCGGTCGTTCGCTAATCTTGCTAAAATTCACGGG | |
| | CCATTAATTAGTCTTAGACTCGGGTCAGTGACTACAATAGTTGTGAGC | |
| | TCTGCCGATGTTGCCAAAGAAATGTTTCTAAAGAAAGATCATCCTCTA | |
| | AGTAATCGTACCATACCCAACAGCGTTACTGCGGGTGATCATCACAAA | |
| | CTAACCATGTCGTGGTTACCAGTGTCTCCCAAGTGGAGGAATTTTCGG | |
| | AAAATAACCGCTGTGCACTTGCTCAGTCCCCAACGGCTCGACGCATGT | |
| | CAAACATTTAGACATGCCAAAGTCCAACAGCTATACGAATATGTTCAA | |
| | GAATGCGCTCAGAAGGGTCAAGCAGTAGACATCGGTAAGGCAGCTTTC | |
| | ACTACAAGTCTTAACCTACTTTCAAAATTATTTTTCTCAGTCGAGTTG | |
| | GCTCACCATAAAAGTCAGACATCACAAGAATTCAAGGAGCTAATCTGG | |
| | AACATAATGGAAGATATCGGTAAACCCAACTATGCAGATTATTTTCCT | |
| | ATATTGGGCTGCGTCGACCCCAGTGGTATACGGAGACGATTGGCATGT | |
| | TCATTTGACAAACTCATTGCTGTTTTTCAGTCTATAATTTGCGAAAGA | |
| | CTAGCTCCGGATAGTTCCACAGCAACTACAACCACAACTGACGATGTG | |
| | CTTGATGTGTTGCTGCAGTTGTTCAAGCAAAATGAGTTAACAATGGGT | |
| | GAGATTAATCACCTTCTGGTGGATATATTCGACGCCGGTACTGATACT | |
| | ACATCGTCTACTTTTGAGTGGGTGATGGCTGAGCTTATCAGGAACCCA | |
| | GAAATGATGGAAAAAGCACAAGAAGAGATAAAACAAGTGTTGGGGAAA | |
| | GATAAACAAATCCAAGAGAGTGATATAATCAACTTGCCTTATCTGCAA | |
| | GCAATAATCAAGGAGACACTACGTCTACACCCTCCCACAGTCTTTCTC | |
| | TTACCACGGAAAGCCGACACCGACGTGGAACTGTACGGATATATAGTT | |
| | CCGAAAGATGCTCAAATTTTAGTCAACCTTTGGGCCATTGGGCGCGAT | |
| | CCCAATGCCTGGCAGAATGCCGATATTTTTAGTCCAGAGAGATTTATC | |
| | GGGTGTGAAATCGATGTTAAAGGTAGAGACTTCGGACTCTTACCCTTC | |
| | GGGGCAGGCAGAAGGATATGTCCAGGAATGAACTTAGCCATCCGAATG | |
| | CTAACACTGATGCTAGCAACCCTTCTCCAATTCTTTAATTGGAAGCTA | |
| | GAAGGTGACATCTCGCCCAAAGACCTTGACATGGACGAGAAATTTGGA | |
| | ATTGCTCTTCAAAAAACAAAACCCTTAAAACTTATACCCATTCCCAGA | |
| | TATTAA | |
| CYP76AD1 | ATGGACCATGCGACCCTTGCAATGATACTAGCCATTTGGTTCATCAGT | 131 |
| (4) | TTTCACTTCATTAAACTACTCTTTAGCCAGCAAACCACTAAACTCCTA | |
| | CCACCGGGTCCCAAACCATTGCCAATCATAGGAAACATCCTTGAGGTG | |
| | GGAAAGAAACCACACCGGTCGTTCGCTAATCTTGCTAAAATTCACGGG | |
| | CCATTAATTAGTCTTAGACTCGGGTCAGTGACTACAATAGTTGTGAGC | |
| | TCTGCCGATGTTGCCAAAGAAATGTTTCTAAAGAAAGATCATCCTCTA | |
| | AGTAATCGTACCATACCCAACAGCGTTACTGCGGGTGATCATCACAAA | |
| | CTAACCATGTCGTGGTTACCAGTGTCTCCCAAGTGGAGGAATTTTCGG | |
| | AAAATAACCGCTGTGCACTTGCTCAGTCCCCAACGGCTCGACGCATGT | |
| | CAAACATTTAGACATGCCAAAGTCCAACAGCTATACGAATATGTTCAA | |
| | GAATGCGCTCAGAAGGGTCAAGCAGTAGACATCGGTAAGGCAGCTTTC | |
| | ACTACAAGTCTTAACCTACTTTCAAAATTATTTTTCTCAGTCGAGTTG | |
| | GCTCACCATAAAAGTCAGACATCACAAGAATTCAAGGAGCTAATCTGG | |
| | AACATAATGGAAGATATCGGTAAACCCAACTATGCAGATTATTTTCCT | |
| | ATATTGGGCTGCGTCGACCCCAGTGGTATACGGAGACGATTGGCATGT | |
| | TCATTTGACAAACTCATTGCTGTTTTTCAGTCTATAATTTGCGAAAGA | |
| | CTAGCTCCGGATAGTTCCACAGCAACTACAACCACAACTGACGATGTG | |
| | CTTGATGTGTTGCTGCAGTTGTTCAAGCAAAATGAGTTAACAATGGGT | |
| | GAGATTAATCACCTTCTGGTGGATATATTCGACGCCGGTACTGATACT | |
| | ACATCGTCTACTTTTGAGTGGGTGATGGCTGAGCTTATCAGGAACCCA | |
| | GAAATGATGGAAAAAGCACAAGAAGAGATAAAACAAGTGTTGGGGAAA | |
| | GATAAACAAATCCAAGAGAGTGATATAATCAACTTGCCTTATCTGCAA | |
| | GCAATAATCAAGGAGACACTACGTCTACACCCTCCCACAGTCTTTCTC | |
| | TTACCACGGAAAGCCGACACCGACGTGGAACTGTACGGATATATAGTT | |
| | CCGAAAGATGCTCAAATTTTAGTCAACCTTTGGGCCATTGGGCGCGAT | |
| | CCCAATGCCTGGCAGAATGCCGATATTTTTAGTCCAGAGAGATTTATC | |
| | GGGTGTGAAATCGATGTTAAAGGTAGAGACTTCGGACTCTTACCCTTC | |
| | GGGGCAGGCAGAAGGATATGTCCAGGAATGAACTTAGCCATCCGAATG | |
| | CTAACACTGATGCTAGCAACCCTTCTCCAATTCTTTAATTGGAAGCTA | |
| | GAAGGTGACATCTCGCCCAAAGACCTTGACATGGACGAGAAATTTGGA | |
| | ATTGCTCTTCAAAAAACAAAACCCTTAAAACTTATACCCATTCCCAGA | |
| | TAT | |
| cDOPA5GT | ATGACTGCAATCAAAATGAATACGAATGGTGAGGGTGAAACCCAACAT | 7 |
| | ATACTTATGATACCATTTATGGCCCAAGGGCACCTTCGACCGTTTCTG | |
| | GAATTGGCCATGTTCTTATACAAACGATCACATGTCATCATAACTCTG | |
| | CTAACAACTCCCCTTAACGCTGGGTTCCTGAGACATCTGTTGCATCAC | |
| | CATTCCTACAGCTCCTCTGGGATACGGATCGTAGAATTGCCGTTTAAT | |
| | TCGACTAATCACGGGCTCCCACCCGGTATAGAAAATACAGACAAATTA | |
| | ACACTGCCTTTAGTCGTATCCTTGTTTCATTCTACAATTTCCCTAGAC | |
| | CCCCACCTACGCGATTATATTTCGAGACACTTCAGTCCTGCACGACCC | |
| | CCACTGTGTGTTATTCACGATGTTTTTCTCGGTTGGGTGGACCAAGTA | |
| | GCTAAGGATGTAGGGTCCACAGGCGTCGTTTTTACGACCGGCGGGGCT | |
| | TATGGCACCTCTGCATATGTATCTATATGGAATGACTTACCACACCAA | |
| | AATTACTCCGACGATCAGGAGTTTCCATTGCCAGGATTCCCCGAGAAT | |
| | CATAAATTTCGAAGATCTCAACTGCATCGTTTCCTTAGGTATGCTGAT | |
| | GGGTCTGATGACTGGTCTAAATATTTCCAACCCCAACTACGGCAAAGT | |
| | ATGAAAAGTTTTGGGTGGCTATGCAATAGTGTGGAAGAGATTGAAACA | |
| | TTGGGCTTCTCGATTCTAAGAAACTACACCAAGCTTCCAATCTGGGGC | |
| | ATAGGCCCACTTATAGCCTCCCCGGTACAGCACAGTTCAAGTGACAAT | |
| | AACTCTACTGGAGCAGAGTTTGTGCAGTGGTTGAGTCTTAAAGAACCC | |
| | GACAGTGTATTATATATATCTTTCGGGAGTCAAAATACTATTTCACCA | |
| | ACTCAGATGATGGAATTGGCTGCAGGGTTGGAGTCGAGTGAGAAACCG | |
| | TTTCTGTGGGTTATACGTGCCCCATTTGGGTTTGATATTAATGAGGAA | |
| | ATGCGTCCCGAATGGCTTCCCGAGGGGTTTGAAGAGAGGATGAAAGTC | |
| | AAAAAGCAAGGGAAGCTTGTTTACAAATTGGGGCCTCAATTGGAAATC | |
| | CTTAATCACGAAAGTATCGGGGGTTTTCTAACGCATTGCGGGTGGAAC | |
| | TCTATTCTTGAGTCCCTAAGAGAAGGTGTTCCCATGTTGGGATGGCCA | |
| | CTAGCAGCTGAACAAGCTTACAACCTAAAATATCTAGAGGATGAAATG | |
| | GGTGTAGCAGTCGAACTAGCCAGAGGCCTCGAGGGGGAAATTTCCAAA | |
| | GAAAAGGTTAAGAGAATAGTTGAGATGATCTTAGAGCGGAATGAAGGT | |
| | TCAAAAGGATGGGAGATGAAAAACAGGGCAGTGGAAATGGGGAAAAAG | |
| | TTAAAAGATGCCGTCAACGAGGAAAAAGAGTTGAAAGGTTCTTCAGTC | |
| | AAAGCAATAGACGATTTCCTTGACGCTGTGATGCAAGCAAAGCTCGAA | |
| | CCGTCATTGCAATGA | |
| AroG^{L175Q} | ATGAATTACCAGAACGATGACTTAAGAATCAAAGAAATTAAAGAGTTG | 132 |
| | TTACCACCTGTGGCTCTTTTGGAAAAATTCCCAGCAACTGAGAATGCT | |
| | GCAAACACAGTTGCTCATGCAAGAAAGGCTATTCACAAAATCTTGAAG | |
| | GGTAATGATGACAGGTTACTTGTTGTGATCGGACCATGCTCAATACAT | |
| | GATCCTGTTGCTGCAAAGGAATACGCTACTAGATTGCTTGCATTGAGG | |
| | GAAGAGTTAAAGGATGAACTTGAGATTGTTATGAGAGTGTACTTCGAG | |
| | AAACCAAGGACCACTGTTGGTTGGAAGGGACTTATCAATGATCCTCAC | |
| | ATGGACAACTCCTTCCAAATTAATGATGGTTTGAGAATCGCTAGGAAA | |
| | CTTTTGCTTGATATTAACGACTCAGGTTTGCCAGCTGCAGGAGAATTT | |
| | TTAGATATGATCACACCTCAGTACTTAGCTGACCTTATGTCATGGGGT | |
| | GCTATAGGAGCAAGAACAACCGAAAGTCAAGTTCATAGGGAGCAGGCT | |
| | TCCGGTTTGTCTTGTCCAGTGGGATTCAAAAATGGTACTGATGGAACA | |
| | ATTAAGGTTGCTATAGACGCAATTAACGCTGCAGGTGCTCCTCATTGT | |
| | TTTCTTTCTGTTACAAAATGGGGACACTCAGCAATCGTGAATACCAGT | |
| | GGTAACGGAGATTGCCATATTATCTTGAGAGGTGGAAAAGAACCAAAT | |
| | TATTCAGCTAAGCACGTTGCAGAAGTGAAAGAGGGTTTGAACAAGGCT | |
| | GGATTACCTGCACAAGTTATGATCGATTTCTCTCATGCTAACTCCTCT | |
| | AAGCAATTCAAGAAACAGATGGATGTTTGTGCTGACGTGTGCCAACAG | |
| | ATCGCTGGTGGAGAAAAGGCTATTATTGGTGTTATGGTGGAAAGTCAC | |
| | TTAGTTGAGGGAAATCAATCATTAGAAAGTGGAGAGCCTCTTGCTTAC | |
| | GGAAAATCTATTACCGATGCATGCATCGGTTGGGAAGATACTGACGCT | |
| | CTTTTGAGACAGTTGGCTAACGCAGTTAAGGCAAGAAGGGGTTGA | |

### Pigment-Producing Polypeptide Sequences

Among other things, the present disclosure provides pigment-producing polypeptides encoded by pigment-producing genes. In some embodiments, a pigment-producing polypeptide may refer to a betalain-related polypeptide or a betalain biosynthesis pathway polypeptide.

In some embodiments, a pigment-producing polypeptide is or comprises a cytochrome P450 polypeptide. In some embodiments, a pigment-producing polypeptide is or comprises a CYP76AD1 polypeptide, a CYP76AD5 polypeptide, a CYP76AD6 polypeptide, or a CYP76AD15 polypeptide. In some embodiments, a pigment-producing polypeptide is or comprises a CYP76AD1 polypeptide.

In some embodiments, a pigment-producing polypeptide is or comprises a DODA polypeptide. In some embodiments, a pigment-producing polypeptide is or comprises a DOPA5GT polypeptide. In some embodiments, a pigment-producing polypeptide is or comprises a cDOPA5GT polypeptide.

In some embodiments, a pigment-producing polypeptide is or comprises an arogenate dehydrogenase (ADH) polypeptide. In some embodiments, a pigment-producing polypeptide is or comprises an ADHα polypeptide.

In some embodiments, a pigment-producing polypeptide is or comprises a 3-deoxy-D-arabino-heptulosonate 7-phosphate synthase (DHAP synthase) polypeptide. In some embodiments, a pigment-producing polypeptide is or comprises a DHAP synthase comprising an L175Q mutation (AroG^{L175Q}).

In some embodiments, a pigment-producing polypeptide is at least 85%, at least 90%, at least 91%, at least 92%, at least 93%, at least 94%, at least 95%, at least 96%, at least 97%, at least 98%, or at least 99% identical to a sequence included in Table 2. In some embodiments, a pigment-producing polypeptide described herein has a polypeptide sequence provided in Table 2.

TABLE 2
Amino Sequences of Exemplary Betalain-Related Polypeptides

| | | SEQ |
|---|---|---|
| Description | Sequence | ID NO: |
| ADHα | MISLSSFHPSSTTATATAAAATTHPPQQCPAFSSPPSHLSLPLRHPRQ | 8 |
| | HLVVRCGGGGSASESVFNRDSAATRVSNDHLDVSKRDVKLKIAIIGFG | |
| | NFGQFLAKTMAKQGHRVLAYSRSDYSRAAKEIGVEYFTDADDLCEEHP | |
| | EVILLCTSILSTEKVLRSLPLHRLRRSTLFADVLSVKEFPRSLFLQLL | |
| | PKDFDILCTHPMFGPDSGKDGWGGLPFVFDKVRVGSDQSRTSRAEAFL | |
| | DVFRNAGCRMVEMSCVDHDKHAAGSQFITHMMGRVLEKLALENTPINT | |
| | KGYESLLNLVDNTARDSFELFYGLFLYNKNAMEQLDRMDWAFEMVKKQ | |
| | LSGYLHDLVRKQLMLEGNNDQAEVTFDKPLMLPSPTINPPQIVPSADM | |
| | AEKKHDLVVVNGTR | |
| DODA | MKMMNGEDANDQMIKESFFITHGNPILTVEDTHPLRPFFETWREKIFS | 9 |
| | KKPKAILIISGHWETVKPTVNAVHINDTIHDFDDYPAAMYQFKYPAPG | |
| | APELARKVEEILKKSGFETAETDEKRGLDHGAWVPLMLMYPEADIPVC | |
| | QLSVQPHLDGTYHYNLGRALAPLKNDGVLIIGSGSATHPLDETPHYFD | |
| | GVAPWAAAFDSWLRKALINGRFEEVNIYETKAPNWKLAHPFPEHFYPL | |
| | HVVLGAAGEKWKAELIHSSWDHGTLCHGSYKFTSA | |
| CYP76AD1 | MDHATLAMILAIWFISFHFIKLLFSQQTTKLLPPGPKPLPIIGNILEV | 10 |
| | GKKPHRSFANLAKIHGPLISLRLGSVTTIVVSSADVAKEMFLKKDHPL | |
| | SNRTIPNSVTAGDHHKLTMSWLPVSPKWRNFRKITAVHLLSPQRLDAC | |
| | QTFRHAKVQQLYEYVQECAQKGQAVDIGKAAFTTSLNLLSKLFFSVEL | |
| | AHHKSQTSQEFKELIWNIMEDIGKPNYADYFPILGCVDPSGIRRRLAC | |
| | SFDKLIAVFQSIICERLAPDSSTATTTTTDDVLDVLLQLFKQNELTMG | |
| | EINHLLVDIFDAGTDTTSSTFEWVMAELIRNPEMMEKAQEEIKQVLGK | |
| | DKQIQESDIINLPYLQAIIKETLRLHPPTVFLLPRKADTDVELYGYIV | |
| | PKDAQILVNLWAIGRDPNAWQNADIFSPERFIGCEIDVKGRDFGLLPF | |
| | GAGRRICPGMNLAIRMLTLMLATLLQFFNWKLEGDISPKDLDMDEKFG | |
| | IALQKTKPLKLIPIPRY | |
| cDOPA5GT | MTAIKMNTNGEGETQHILMIPFMAQGHLRPFLELAMFLYKRSHVIITL | 11 |
| | LTTPLNAGFLRHLLHHHSYSSSGIRIVELPFNSTNHGLPPGIENTDKL | |
| | TLPLVVSLFHSTISLDPHLRDYISRHFSPARPPLCVIHDVFLGWVDQV | |
| | AKDVGSTGVVFTTGGAYGTSAYVSIWNDLPHONYSDDQEFPLPGFPEN | |
| | HKFRRSQLHRFLRYADGSDDWSKYFQPOLRQSMKSFGWLCNSVEEIET | |
| | LGFSILRNYTKLPIWGIGPLIASPVQHSSSDNNSTGAEFVQWLSLKEP | |
| | DSVLYISFGSQNTISPTOMMELAAGLESSEKPFLWVIRAPFGFDINEE | |
| | MRPEWLPEGFEERMKVKKQGKLVYKLGPQLEILNHESIGGFLTHCGWN | |
| | SILESLREGVPMLGWPLAAEQAYNLKYLEDEMGVAVELARGLEGEISK | |
| | EKVKRIVEMILERNEGSKGWEMKNRAVEMGKKLKDAVNEEKELKGSSV | |
| | KAIDDFLDAVMQAKLEPSLQ | |
| CYP76AD1 | MDHATLAMILAIWFISFHFIKLLFSQQTTKLLPPGPKPLPIIGNILEV | 12 |
| (2) | GKKPHRSFANLAKIHGPLISLRLGSVTTIVVSSADVAKEMFLKKDHPL | |
| | SNRTIPNSVTAGDHHKLTMSWLPVSPKWRNFRKITAVHLLSPQRLDAC | |
| | QTFRHAKVQQLYEYVQECAQKGQAVDIGKAAFTTSLNLLSKLFFSVEL | |
| | AHHKSHTSQEFKELIWNIMEDIGKPNYADYFPILGCVDPSGIRRRLAC | |
| | SFDKLIAVFQGIICERLAPDSSTTTTTTTDDVLDVLLQLFKQNELTMG | |
| | EINHLLVDIFDAGTDTTSSTFEWVMTELIRNPEMMEKAQEEIKQVLGK | |
| | DKQIQESDIINLPYLQAIIKETLRLHPPTVFLLPRKADTDVELYGYIV | |
| | PKDAQILVNLWAIGRDPNAWQNADIFSPERFIGCEIDVKGRDFGLLPF | |
| | GAGRRICPGMNLAIRMLTLMLATLLQFFNWKLEGDISPKDLDMDEKFG | |
| | IALQKTKPLKLIPIPRY | |
| DODA (2) | MKMMNGEDANDQMIKESFFITHGNPILTVEDTHPLRPFFETWREKIFS | 13 |
| | KKPKAILIISGHWETVKPTVNAVHINDTIHDFDDYPAAMYQFKYPAPG | |
| | EPELARKVEEILKKSGFETAETDQKRGLDHGAWVPLMLMYPEADIPVC | |
| | QLSVQPHLDGTYHYNLGRALAPLKNDGVLIIGSGSATHPLDETPHYFD | |
| | GVAPWAAAFDSWLRKALINGRFEEVNIYESKAPNWKLAHPFPEHFYPL | |
| | HVVLGAAGEKWKAELIHSSWDHGTLCHGSYKFTSA | |
| DOPA | MTAIKMNTNGEGETQHILMIPFMAQGHLRPFLELAMFLYKRSHVIITL | 14 |
| | LTTPLNAGFLRHLLHHHSYSSSGIRIVELPFNSTNHGLPPGIENTDKL | |
| | TLPLVVSLFHSTISLDPHLRDYISRHFSPARPPLCVIHDVFLGWVDQV | |
| | AKDVGSTGVVFTTGGAYGTSAYVSIWNDLPHONYSDDQEFPLPGFPEN | |
| | HKFRRSQLHRFLRYADGSDDWSKYFQPQLRQSMKSFGWLCNSVEEIET | |
| | LGFSILRNYTKLPIWGIGPLIASPVQHSSSDNNSTGAEFVQWLSLKEP | |
| | DSVLYISFGSQNTISPTOMMELAAGLESSEKPFLWVIRAPFGFDINEE | |
| | MRPEWLPEGFEERMKVKKQGKLVYKLGPQLEILNHESIGGFLTHCGWN | |
| | SILESLREGVPMLGWPLAAEQAYNLKYLEDEMGVAVELARGLEGEISK | |
| | EKVKRIVEMILERNEGSKGWEMKNRAVEMGKKLKDAVNEEKELKGSSV | |
| | KAIDDFLDAVMQAKLEPSLQ | |
| BvCYP76AD1 | MDHATLAMILAIWFISFHFIKLLFSQQTTKLLPPGPKPLPIIGNILEV | 15 |
| | GKKPHRSFANLAKIHGPLISLRLGSVTTIVVSSADVAKEMFLKKDHPL | |
| | SNRTIPNSVTAGDHHKLTMSWLPVSPKWRNFRKITAVHLLSPQRLDAC | |
| | QTFRHAKVQQLYEYVQECAQKGQAVDIGKAAFTTSLNLLSKLFFSVEL | |
| | AHHKSQTSQEFKELIWNIMEDIGKPNYADYFPILGCVDPSGIRRRLAC | |
| | SFDKLIAVFQSIICERLAPDSSTATTTTTDDVLDVLLQLFKQNELTMG | |
| | EINHLLVDIFDAGTDTTSSTFEWVMAELIRNPEMMEKAQEEIKQVLGK | |
| | DKQIQESDIINLPYLQAIIKETLRLHPPTVFLLPRKADTDVELYGYIV | |
| | PKDAQILVNLWAIGRDPNAWQNADIFSPERFIGCEIDVKGRDFGLLPF | |
| | GAGRRICPGMNLAIRMLTLMLATLLQFFNWKLEGDISPKDLDMDEKFG | |
| | IALQKTKPLKLIPIPRY | |
| BvCYP76AD1 | MDHATLAMILAILFISFHFIKLLFSQQTTKLLPPGPKPLPIIGNILEV | 16 |
| W13L | GKKPHRSFANLAKIHGPLISLRLGSVTTIVVSSADVAKEMFLKKDHPL | |
| | SNRTIPNSVTAGDHHKLTMSWLPVSPKWRNFRKITAVHLLSPQRLDAC | |
| | QTFRHAKVQQLYEYVQECAQKGQAVDIGKAAFTTSLNLLSKLFFSVEL | |
| | AHHKSQTSQEFKELIWNIMEDIGKPNYADYFPILGCVDPSGIRRRLAC | |
| | SFDKLIAVFQSIICERLAPDSSTATTTTTDDVLDVLLQLFKQNELTMG | |
| | EINHLLVDIFDAGTDTTSSTFEWVMAELIRNPEMMEKAQEEIKQVLGK | |
| | DKQIQESDIINLPYLQAIIKETLRLHPPTVFLLPRKADTDVELYGYIV | |
| | PKDAQILVNLWAIGRDPNAWQNADIFSPERFIGCEIDVKGRDFGLLPF | |
| | GAGRRICPGMNLAIRMLTLMLATLLQFFNWKLEGDISPKDLDMDEKFG | |
| | IALQKTKPLKLIPIPRY | |
| BvDODA | MKMMNGEDANDQMIKESFFITHGNPILTVEDTHPLRPFFETWREKIFS | 17 |
| | KKPKAILIISGHWETVKPTVNAVHINDTIHDFDDYPAAMYQFKYPAPG | |
| | APELARKVEEILKKSGFETAETDEKRGLDHGAWVPLMLMYPEADIPVC | |
| | QLSVQPHLDGTYHYNLGRALAPLKNDGVLIIGSGSATHPLDETPHYFD | |
| | GVAPWAAAFDSWLRKALINGRFEEVNIYETKAPNWKLAHPFPEHFYPL | |
| | HVVLGAAGEKWKAELIHSSWDHGTLCHGSYKFTSA | |
| MjDOPA5GT | MTAIKMNTNGEGETQHILMIPFMAQGHLRPFLELAMFLYKRSHVIITL | 18 |
| | LTTPLNAGFLRHLLHHHSYSSSGIRIVELPFNSTNHGLPPGIENTDKL | |
| | TLPLVVSLFHSTISLDPHLRDYISRHFSPARPPLCVIHDVFLGWVDQV | |
| | AKDVGSTGVVFTTGGAYGTSAYVSIWNDLPHONYSDDQEFPLPGFPEN | |
| | HKFRRSQLHRFLRYADGSDDWSKYFQPQLRQSMKSFGWLCNSVEEIET | |
| | LGFSILRNYTKLPIWGIGPLIASPVQHSSSDNNSTGAEFVQWLSLKEP | |
| | DSVLYISFGSQNTISPTOMMELAAGLESSEKPFLWVIRAPFGFDINEE | |
| | MRPEWLPEGFEERMKVKKQGKLVYKLGPQLEILNHESIGGFLTHCGWN | |
| | SILESLREGVPMLGWPLAAEQAYNLKYLEDEMGVAVELARGLEGEISK | |
| | EKVKRIVEMILERNEGSKGWEMKNRAVEMGKKLKDAVNEEKELKGSSV | |
| | KAIDDFLDAVMQAKLEPSLQ | |
| AmCYP76AD1 | MDNATLAMILTIWLISINFIKMFFTHQNTKLILPPGPKPLPIIGNILE | 19 |
| | VGKKPHRSFANLAKIHGPLISLRLGSVTTIVVSSAEVAKEMFLKKDQP | |
| | LSNRNVPNSVTAGDHHKLTMSWLPVSPKWRNFRKITAVHLLSPLRLDA | |
| | COSLRQAKVOQLYQYVQECAQKGQSIDIGKAAFTTSLNLLSKLFFSKE | |
| | LACHKSHESQEFKQLIWNIMEDIGKPNYADYFPILGCIDPLGIRRRLA | |
| | ANFDKLIAVFQTIISERLENNKNANATNDVLDVLLQLYKQKELSMGEI | |
| | NHLLVDIFDAGTDTTSSTFEWVMTELIRNPNMMEKAQQEIQEVLGKDR | |
| | QIQESDIIKLPYLQALIKETLRLHPPTVFLLPRKADMDVELYGYVVPK | |
| | DAQILVNLWAIGRDPQAWEKPNAFLPERFLGSDVDVKGRDFGLLPFGA | |
| | GKRICPGMNLAIRMLTLMLATLLQFFDWKLEEGMNPQDLDMDEKFGIA | |
| | LOKTKPLEIIPSRRH | |
| AmDODA | MVEKEMNFNETFFITHGNPILTVEDSHPLRPFFETWKQNIFSKKPKAI | 20 |
| | LVISGHWETDFPSVNAVDINDTIHDFDDYPDAMYQLKYPAPGSIEMAK | |
| | KVEEILTKSGYKTINMDKKRGLDHGAWVPLMYMYPQADIPVCQLSVQP | |
| | KLSGTYHYNLGRALAPLKQEGVLIIGSGSATHPLDETPHFHNGVAPWA | |
| | HEFHSWLHSALINRRIEEVNTYESKAPNWELAHPFPEHFYPLHIVMGA | |
| | AGDNWKAELIHSSWDHGTLYHGAYKFTSN | |
| AmDOPA | MDSSKTKHIILLPFLAQGHLRPFLHLANFLRSHTPFTLSILTTPLNAA | 21 |
| | NLRRQSNNINIYDLPFNSTDHNLPPNTENTEKLPLTSIISLFYASTSL | |
| | QPHVRNHLTRHHLDNPPICIIFDIFLGWADNLARSIGSTGVCFNTGGA | |
| | YGLAAYMSIWMHLPHRNVPDNVEFSLPEFPENRKFKRNQLHRFLRFAD | |
| | GTDDWSGFFQPQIKYSRNCSAWLCNSIEEIEPLGFEVLRAMLKVPVWG | |
| | IGPLVKTENSCDEDDEQGCVEWLNQFEKGSVLYISFGSQNTVTPIQMM | |
| | ELAKGLEESGAKFLWVIRPPFGFDINGEFKPEWLPEGFEKRVMERKQG | |
| | KLVKKWGPQMEILRNKATGAFLSHCGWNSLIEALSEGVPIIGWPLAAE | |
| | QAYNSKMLVEEMGVAVELTRGLEGELSSEGVKKVVEMVMDREEGSFGC | |
| | EMKKAAVVIGQKLRDAMKVEGDYRGCSLRSLDEFVEFIVSRKATLS | |
| CqCYP76AD1 | MDHATLAMILAIWFVVFHFIKMLFTSQTTKLLPPGPKPLPLIGNILEV | 22 |
| | GEKPHQSFANLAKIHGPLISLRLGSVTTIVVSSAEVAKEMFLKKDHPL | |
| | SNRTVPNSVTAGDHHKLTMSWLPVSPKWRNFRKITAVHLLSPQRLDAC | |
| | QTLRHAKVQQLFQYVQECAQKGQAVDIGKAAFTTSLNLLSKLFFSVEL | |
| | AHHKSHTSQQFKELIWNIMEDIGKPNYADYFPILGCLDPSGIRRRLAS | |
| | NFDKLIAVFQSIICQRIGNGQDSASTKTTDDVLDILLDLHKQKELSMG | |
| | EINHLLVDIFDAGTDTTSSTFEWVMAELIRNPKMMEKAQEEIEQVLGK | |
| | DRQIQESDIIKLPYLQAIIKETLRLHPPTVFLLPRKADSDVELYGYVV | |
| | PKDAQILVNLWAIGRDPQAWVKPDVFLPERFLGSEIDVKGRDFGLLPF | |
| | GAGRRICPGMNLAIRMLTLMLATLLQFFNWKLEEGMKAEDLDMDEKFG | |
| | IALQKTKPLQIIPVLRY | |
| CqCYP75AD1 | MDHATLAMILAILFVVFHFIKMLFTSQTTKLLPPGPKPLPLIGNILEV | 23 |
| W13L | GEKPHQSFANLAKIHGPLISLRLGSVTTIVVSSAEVAKEMFLKKDHPL | |
| | SNRTVPNSVTAGDHHKLTMSWLPVSPKWRNFRKITAVHLLSPQRLDAC | |
| | QTLRHAKVQQLFQYVQECAQKGQAVDIGKAAFTTSLNLLSKLFFSVEL | |
| | AHHKSHTSQQFKELIWNIMEDIGKPNYADYFPILGCLDPSGIRRRLAS | |
| | NFDKLIAVFQSIICQRIGNGQDSASTKTTDDVLDILLDLHKQKELSMG | |
| | EINHLLVDIFDAGTDTTSSTFEWVMAELIRNPKMMEKAQEEIEQVLGK | |
| | DRQIQESDIIKLPYLQAIIKETLRLHPPTVFLLPRKADSDVELYGYVV | |
| | PKDAQILVNLWAIGRDPQAWVKPDVFLPERFLGSEIDVKGRDFGLLPF | |
| | GAGRRICPGMNLAIRMLTLMLATLLQFFNWKLEEGMKAEDLDMDEKFG | |
| | IALQKTKPLQIIPVLRY | |
| CqDODA | MKMSGEDNSMIKETFFITHGNPILTVEDTHPLRPFFETWREKIFTMKP | 24 |
| | KAILVISGHWETDHPAVNAVHINDTIYDFDDYPQAMYKLKYPAPGSPD | |
| | LASRVEEIMKKSGFDTVHIDKKRGLDHGAWVPLMYMYPEADIPVCQLS | |
| | VQPKLDGTYHYNLGRALAPLRDEGVLIIGSGSATHPLDETPHYHGGVA | |
| | PWAADFDSWLDVALTKGRFEEVNTYETKAPNWELAHPFPEHFYPLHVV | |
| | VGAAGEKWKAELIHTSWDHGTLCHGSYKFTST | |
| CqUDP92A1 | MESSTKEQEQQHIILLPFLAQGHLRPFLHLAHRLLSLTPFNLSLLTTP | 25 |
| L79V | LNAANLRRQSDNFNINLNIIELPFTSTDHGVPPNTENTDKLSLTSIIK | |
| | LFHASTSLEPHVRDYLTRHHLHDPPVCIIFDVFLGWADNVARSVGSTG | |
| | ICFNTGGAYGVGAYVSIWSNLPHRNVGDDEEFSLAGFPEDRKLRRNQL | |
| | HRFLRFADGSDEWSRFFQPQIKSSLNCSGWLCNSIEEIEPLGFQVFRN | |
| | FTKSPIWGIGPLIMTSSKKDDDEKEEACLRWLNQFENDSVLYICFGSQ | |
| | NTVTPIQMMELAKGLEESKIPFLWVIRPPFGFDENGEFKPEWLPEKFE | |
| | ERMMEKKQGMLVRDWVPQLDILRHEATGGFLSHCGWNSVLEGLREGVP | |
| | ILGWPLAAEQAYNSKMMVEEMGVAVELTRGLEGEVKKEWVKSVVEMVL | |
| | DRKEGSCGWEMKKKAVEIGEKLKDAWKIEGDYKGSSVKAMDNFVEFIV | |
| | SCRGKKSKEDA | |
| AroG^{L175Q} | MNYQNDDLRIKEIKELLPPVALLEKFPATENAANTVAHARKAIHKILK | 133 |
| | GNDDRLLVVIGPCSIHDPVAAKEYATRLLALREELKDELEIVMRVYFE | |
| | KPRTTVGWKGLINDPHMDNSFQINDGLRIARKLLLDINDSGLPAAGEF | |
| | LDMITPQYLADLMSWGAIGARTTESQVHREQASGLSCPVGFKNGTDGT | |
| | IKVAIDAINAAGAPHCFLSVTKWGHSAIVNTSGNGDCHIILRGGKEPN | |
| | YSAKHVAEVKEGLNKAGLPAQVMIDFSHANSSKQFKKQMDVCADVCQQ | |
| | IAGGEKAIIGVMVESHLVEGNOSLESGEPLAYGKSITDACIGWEDTDA | |
| | LLRQLANAVKARRG | |

The present disclosure recognizes that certain mutations in an amino acid sequence of a pigment-producing polypeptide or characteristic portion thereof described herein will not impact expression, folding, or activity of the polypeptide. In some embodiments, a pigment-producing polypeptide or characteristic portion thereof includes one or more mutations, where the one or more mutations comprise or are one or more conservative amino acid substitutions.

### Coding Sequence

Among other things, the present disclosure provides polynucleotides comprising a coding sequence. In some embodiments, a coding sequence is or comprises an engineered coding sequence.

In some embodiments, a coding sequence comprises one or more pigment-producing genes. In some embodiments, a coding sequence encodes one or more pigment-producing polypeptides.

In some embodiments, one or more coding sequence encodes one or more pigment-producing polypeptides.

In some embodiments, a polynucleotide comprises one or more coding sequences. In some embodiments, a polynucleotide comprises two or more coding sequences. In some embodiments, a polynucleotide comprises at least one coding sequence. In some embodiments, a polynucleotide comprises at least two coding sequences.

In some embodiments, a polynucleotide comprises a coding sequence comprising a nucleic acid sequence encoding a pigment-producing polypeptide. In some embodiments, a coding sequence comprises a first nucleic acid sequence encoding a first pigment-producing polypeptide. In some embodiments, a coding sequence comprises a second nucleic acid sequence encoding a second pigment-producing polypeptide. In some embodiments, a coding sequence comprises a third nucleic acid sequence encoding a third pigment-producing polypeptide. In some embodiments, a coding sequence comprises a fourth nucleic acid sequence encoding a fourth pigment-producing polypeptide. In some embodiments, a coding sequence comprises a fifth nucleic acid sequence encoding a fifth pigment-producing polypeptide.

In some embodiments, a coding sequence encodes a first pigment-producing polypeptide that is or comprises a cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a coding sequence encodes a first pigment-producing polypeptide that is or comprises CYP76AD1 polypeptide. In some embodiments, a coding sequence encodes a first pigment-producing polypeptide that is or comprises a cyclo-DOPA 5-O-glucosyltransferase (cDOPA5GT) polypeptide. In some embodiments, a coding sequence encodes a first pigment-producing polypeptide that is or comprises a L-DOPA 4,5-dioxygenase (DODA) polypeptide.

In some embodiments, a first pigment-producing polypeptide is or comprises a cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a first pigment-producing polypeptide is or comprises a CYP76AD1 polypeptide. In some embodiments, a first pigment-producing polypeptide is or comprises a cyclo-DOPA 5-O-glucosyltransferase (cDOPA5GT) polypeptide. In some embodiments, a first pigment-producing polypeptide is or comprises a L-DOPA 4,5-dioxygenase (DODA) polypeptide.

In some embodiments, a coding sequence encodes a second pigment-producing polypeptide that is or comprises cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a coding sequence encodes a second pigment-producing polypeptide that is or comprises a CYP76AD1 polypeptide. In some embodiments, a coding sequence encodes a second pigment-producing polypeptide that is or comprises a cyclo-DOPA 5-O-glucosyltransferase (cDOPA5GT) polypeptide. In some embodiments, a coding sequence encodes a second pigment-producing polypeptide that is or comprises a L-DOPA 4,5-dioxygenase (DODA) polypeptide.

In some embodiments, a second pigment-producing polypeptide is or comprises a cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a second pigment-producing polypeptide is or comprises a CYP76AD1 polypeptide. In some embodiments, a second pigment-producing polypeptide is or comprises a cyclo-DOPA 5-O-glucosyltransferase (cDOPA5GT) polypeptide. In some embodiments, a second pigment-producing polypeptide is or comprises a L-DOPA 4,5-dioxygenase (DODA) polypeptide.

In some embodiments, a coding sequence encodes a third pigment-producing polypeptide that is or comprises a cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a coding sequence encodes a third pigment-producing polypeptide that is or comprise a CYP76AD1 polypeptide. In some embodiments, a coding sequence encodes a third pigment-producing polypeptide that is or comprise a cyclo-DOPA 5-O-glucosyltransferase (cDOPA5GT) polypeptide. In some embodiments, a coding sequence encodes a third pigment-producing polypeptide that is or comprise a L-DOPA 4,5-dioxygenase (DODA) polypeptide.

In some embodiments, a third pigment-producing polypeptide is or comprises a cytochrome P450 (CYP) polypeptide, a cyclo-DOPA 5-O-glucosyltransferase (cDOPA5GT) polypeptide, or a L-DOPA 4,5-dioxygenase (DODA) polypeptide. In some embodiments, a third pigment-producing polypeptide is or comprises a CYP76AD1 polypeptide. In some embodiments, a third pigment-producing polypeptide is or comprises a cyclo-DOPA 5-O-glucosyltransferase (cDOPA5GT) polypeptide. In some embodiments, a third pigment-producing polypeptide is or comprises a L-DOPA 4,5-dioxygenase (DODA) polypeptide.

In some embodiments, a coding sequence encodes a fourth pigment-producing polypeptide that is or comprises a 3-deoxy-D-arabino-heptulosonate 7-phosphate synthase (DHAP synthase) polypeptide or an arogenate dehydrogenase (ADH) polypeptide. In some embodiments, a coding sequence encodes a fourth pigment-producing polypeptide that is or comprises an AroG^{L175Q} polypeptide. In some embodiments, a coding sequence encodes a fourth pigment-producing polypeptide that is or comprises an ADHα polypeptide.

In some embodiments, a fourth pigment-producing polypeptide is or comprises a 3-deoxy-D-arabino-heptulosonate 7-phosphate synthase (DHAP synthase) polypeptide or an arogenate dehydrogenase (ADH) polypeptide. In some embodiments, a fourth pigment-producing polypeptide is or comprises an AroG^{L175Q} polypeptide. In some embodiments, a fourth pigment-producing polypeptide is or comprises an ADHα polypeptide.

In some embodiments, a coding sequence encodes a fifth pigment-producing polypeptide that is or comprises a 3-deoxy-D-arabino-heptulosonate 7-phosphate synthase (DHAP synthase) polypeptide or an arogenate dehydrogenase (ADH) polypeptide. In some embodiments, a coding sequence encodes a fifth pigment-producing polypeptide that is or comprises an AroG^{L175Q} polypeptide. In some embodiments, a coding sequence encodes a fifth pigment-producing polypeptide that is or comprises an ADHα polypeptide.

In some embodiments, a fifth pigment-producing polypeptide is or comprises a 3-deoxy-D-arabino-heptulosonate 7-phosphate synthase (DHAP synthase) polypeptide or an arogenate dehydrogenase (ADH) polypeptide. In some embodiments, a fifth pigment-producing polypeptide is or comprises an AroG^{L175Q} polypeptide. In some embodiments, a fifth pigment-producing polypeptide is or comprises an ADHα polypeptide.

In some embodiments, a coding sequence comprises (i) a first nucleic acid sequence encoding a first pigment-producing polypeptide, (ii) a second nucleic acid sequence encoding a second pigment-producing polypeptide, and (iii) a third nucleic acid sequence encoding a third pigment-producing polypeptide.

In some embodiments, a coding sequence comprises (i) a first nucleic acid sequence that encodes a first pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NOs: 10, 12, 15, 16, 19, 22, or 23, (ii) a second nucleic acid sequence that encodes a second pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 9, 13, 17, 20, or 24 and (iii) a third nucleic acid sequence that encodes a third pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11, 14, 18, 21, or 25.

In some embodiments, a coding sequence comprises (i) a first nucleic acid sequence that encodes a first pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12, (ii) a second nucleic acid sequence that encodes a second pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17, and (iii) a third nucleic acid sequence that encodes a third pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11.

In some embodiments, a coding sequence comprises (i) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NOs: 1, 6, 127, or 131, (ii) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5 or 130 and (iii) a third nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2, 3, 7, or 128.

In some embodiments, a coding sequence comprises (i) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 127, (ii) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 130, and (iii) a third nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 128.

In some embodiments, a polynucleotide comprises a first and second coding sequence. In some embodiments, a first coding sequence comprises (a) a first nucleic acid sequence encoding a first pigment-producing polypeptide, (b) a second nucleic acid sequence encoding a second pigment-producing polypeptide, and (c) a third nucleic acid sequence encoding a third pigment-producing polypeptide. In some embodiments, a second coding sequence comprises (a) a fourth nucleic acid sequence encoding a fourth pigment-producing polypeptide, and (b) a fifth nucleic acid sequence encoding a fifth pigment-producing polypeptide.

In some embodiments, a polynucleotide comprises a first and second coding sequence, where (i) a first coding sequence comprising (a) a first nucleic acid sequence that encodes a first pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NOs: 10, 12, 15, 16, 19, 22, or 23, (b) a second nucleic acid sequence that encodes a second pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 9, 13, 17, 20, or 24, and (c) a third nucleic acid sequence that encodes a third pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11, 14, 18, 21, or 25; and (ii) a second coding sequence comprising (a) a fourth nucleic acid sequence that encodes a fourth pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 133, and (b) a fifth nucleic acid sequence that encodes a fifth pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 8.

In some embodiments, a polynucleotide comprises a first and second coding sequence, where (i) a first coding sequence comprising (a) a first nucleic acid sequence that encodes a first pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12, (b) a second nucleic acid sequence that encodes a second pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17, and (c) a third nucleic acid sequence that encodes a third pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11; and (ii) a second coding sequence comprising (a) a fourth nucleic acid sequence that encodes a fourth pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 133, and (b) a fifth nucleic acid sequence that encodes a fifth pigment-producing polypeptide comprising an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 8.

In some embodiments, a polynucleotide comprises a first and second coding sequence, where (i) a first coding sequence comprises (a) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NOs: 1, 6, 127, or 131, (b) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5 or 130, and (c) a third nucleic acid sequence that that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2, 3, 7, or 128; and (ii) a second coding sequence comprises (a) a fourth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 4 or 129, and (b) a fifth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 132.

In some embodiments, a polynucleotide comprises a first and second coding sequence, where (i) a first coding sequence comprises (a) a first nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 127, (b) a second nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 130, and (c) a third nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 128; and (ii) a second coding sequence comprises (a) a fourth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 129, and (b) a fifth nucleic acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 132.

### Self-Cleaving Peptide

In some embodiments, a coding sequence comprises a polynucleotide sequence that encodes a peptide sequence that induces polypeptide cleavage and/or failure to form a peptide linkage during translation. In some embodiments, constructs as described herein may include a self-cleaving peptide, that in some embodiments may be a 2A self-cleaving peptide. In some embodiments, such a peptide is approximately 18 to 22 amino acids in length, e.g., 18 amino acids in length, 19 amino acids in length, 20 amino acids in length, 21 amino acids in length, or 22 amino acids in length. In some embodiments, such a peptide may induce ribosomal skipping during translation of a polypeptide. In some embodiments, a 2A self-cleaving peptide is represented by a core sequence motif of DxExNPGP, and are found endogenously in a range of viral families. In some embodiments, a self-cleaving peptide generates polypeptides from a single transcript by causing the ribosome to fail at making a peptide bond. One skilled in the art will recognize that alternative peptide cleavage sequences exist (self-cleaving or requiring the aid of endogenous cellular machinery), and may be incorporated into constructs as described herein.

In some embodiments, a ‘remnant’ 2A residue appended to the carboxyl terminus of the processed polypeptides can be removed by fusing an engineered mini-intein with the 2A sequence through a linker to create an ‘IntF2A’ self-excising domain. In some embodiments, an IntF2A enables co-translational cleavage via 2A's translational recoding activity, followed by post-translational autocatalytic cleavage via intein at its N-terminal junction (Zhang et al., Plant Biotechnology, 2017; incorporated herein by reference in its entirety).

In some embodiments, a coding sequence comprises one or more nucleic acid sequences that each encode a self-cleaving peptide. In some embodiments, a coding sequence comprises at least one nucleic acid sequence encoding a T2A peptide, a P2A peptide, an E2A peptide, or a F2A peptide.

In some embodiments, a self-cleaving peptide is encoded by a nucleic acid sequence that is at least 85%, at least 90%, at least 91%, at least 92%, at least 93%, at least 94%, at least 95%, at least 96%, at least 97%, at least 98%, or at least 99%, or 100% identical to a sequence included in Table 14.

TABLE 14
Self-Cleaving Peptide Polynucleotide Sequences

| | | SEQ ID |
|---|---|---|
| Description | Sequence | NO: |
| Exemplary | TGTCTATCCTTTGGAACAGAGATATTGACAGTGGAATATGGCCCGTTA | 91 |
| IntF2A | CCAATAGGCAAAATCGTGTCAGAAGAGATCAATTGCTCAGTCTATTCT | |
| nucleotide | GTTGATCCTGAGGGTAGAGTTTATACACAAGCCATTGCGCAATGGCAT | |
| sequence | GATAGAGGCGAACAAGAAGTCTTGGAATATGAATTAGAGGACGGGAGC | |
| | GTCATTAGGGCAACAAGTGATCATAGGTTTCTTACTACAGATTATCAA | |
| | CTTCTCGCCATTGAGGAAATTTTTGCCCGACAGCTAGATCTCCTGACA | |
| | CTCGAAAATATTAAACAAACCGAGGAAGCGTTGGATAATCATCGCCTC | |
| | CCGTTTCCTCTCCTAGATGCAGGGACAATTAAGATGGTTAAAGTGATT | |
| | GGGAGGAGATCACTTGGTGTGCAAAGGATTTTTGATATAGGGCTCCCT | |
| | CAGGACCACAACTTCTTACTGGCTAACGGGGCAATCGCGGCAGCTTGT | |
| | TCATGTGGTAGTGGGTCACGGGTAACTGAGTTACTTTATAGGATGAAG | |
| | CGAGCTGAAACCTATTGCCCAAGACCCCTTTTGGCGATTCATCCTACA | |
| | GAAGCACGCCACAAACAAAAAATTGTGGCCCCAGTTAAACAACTTCTC | |
| | AATTTTGACCTTTTGAAGTTGGCCGGTGACGTCGAATCTAACCCCGGC | |
| | CCT | |
| Self- | GGTAGCGGAGCTACCAATTTTAGCCTCCTTAAGCAGGCAGGTGATGTA | 92 |
| Cleaving 2A | GAAGAGAACCCCGGGCC | |
| Peptide P2A- | | |
| 4 | | |
| polynucleotide | | |
| sequence | | |
| 1 | | |
| Self- | GGATCCGGAGCAACCAACTTTAGCCTGCTCAAGCAAGCAGGAGATGTT | 93 |
| Cleaving 2A | GAGGAAAATCCTGGCCCC | |
| Peptide P2A- | | |
| 4 | | |
| polynucleotide | | |
| sequence | | |
| 2 | | |
| Self- | GGTAGCGGAGCTACCAATTTTAGCCTCCTTAAGCAGGCAGGTGATGTA | 147 |
| Cleaving 2A | GAAGAGAACCCCGGGCCT | |
| Peptide P2A | | |
| polynucleotide | | |
| sequence | | |
| 3 | | |
| Pisum | GCTTCTATGATATCCTCTTCAGCTGTGACTACAGTCAGCCGTGCTTCT | 148 |
| sativum | ACGGTGCAATCGGCCGCGGTGGCTCCATTCGGCGGCCTCAAATCCATG | |
| chloroplast | ACTGGATTCCCAGTTAAGAAGGTCAACACTGACATTACTTCCATTACA | |
| ribulose- | AGCAATGGTGGAAGAGTAAAGTGC | |
| 1,5- | | |
| bisphosphate | | |
| carboxylase/ | | |
| oxygenase | | |
| small | | |
| subunit | | |
| signal | | |
| peptide | | |
| (PeaChlSP) | | |
| polynucleotide | | |
| sequence | | |
| A. thaliana | ATGGCTTCCTCTATGCTCTCCTCTGCCGCTGTGGTTACCTCCCCGGCT | 187 |
| ribulose- | CAAGCCACCATGGTCGCTCCATTCACTGGTTTGAAGTCATCCGCTTCT | |
| 1,5- | TTCCCGGTCACCCGCAAGGCCAACAACGACATTACTTCCATCACAAGC | |
| biphosphate | AATGGGGGAAGAGTTAGCTGC | |
| carboxylase | | |
| small | | |
| subunit | | |
| (rbcS) | | |
| Choroplastic | | |
| signal | | |
| peptide | | |

In some embodiments, a self-cleaving peptide is or comprises an amino acid sequence that is at least 85%, at least 90%, at least 91%, at least 92%, at least 93%, at least 94%, at least 95%, at least 96%, at least 97%, at least 98%, or at least 99%, or 100% identical to a sequence included in Table 15.

TABLE 15
Self-Cleaving Peptide Polypeptide Sequences

| | | SEQ |
|---|---|---|
| Description | Sequence | ID NO: |
| Exemplary | GSGEGRGSLLTCGDVEENPGP | 97 |
| Cleavage | | |
| signal | | |
| amino acid | | |
| sequence | | |
| Exemplary | APVKQTLNFDLLKLAGDVESNPGP | 98 |
| Cleavage | | |
| signal | | |
| amino acid | | |
| sequence | | |
| Exemplary | CLSFGTEILTVEYGPLPIGKIVSEEINCSVYSVDPEGRVYTQAIAQWH | 99 |
| IntF2A | DRGEQEVLEYELEDGSVIRATSDHRFLTTDYQLLAIEEIFARQLDLLT | |
| amino acid | LENIKQTEEALDNHRLPFPLLDAGTIKMVKVIGRRSLGVQRIFDIGLP | |
| sequence | QDHNFLLANGAIAAACSCGSGSRVTELLYRMKRAETYCPRPLLAIHPT | |
| | EARHKQKIVAPVKQLLNFDLLKLAGDVESNPGP | |
| Self- | GSGATNFSLLKQAGDVEENPGP | 149 |
| Cleaving 2A | | |
| Peptide P2A | | |
| polypeptide | | |
| sequence | | |
| PeaChlSP | MASMISSSAVTTVSRASTVQSAAVAPFGGLKSMTGFPVKKVNTDITSI | 150 |
| signal | TSNGGRVKC | |
| peptide | | |
| polypeptide | | |
| sequence | | |
| A. thaliana | MASSMLSSAAVVTSPAQATMVAPFTGLKSSASFPVTRKANNDITSITS | 188 |
| ribulose- | NGGRVSC | |
| 1,5- | | |
| biphosphate | | |
| carboxylase | | |
| small | | |
| subunit | | |
| (rbcS) | | |
| Choroplastic | | |
| signal | | |
| peptide | | |

In some embodiments, a coding sequence provided herein includes a linker peptide. In some embodiments, a linker peptide is utilized to join two or more functional peptides in a translational product. In some embodiments, such a linker peptide may include additional functional sequences, such as recognition sequences for endogenous peptidases. In some embodiments, a linker peptide may fuse two polypeptides together indefinitely. In some embodiments, a linker peptide sequence may be one amino acid in length, two amino acids in length, three amino acids in length, four amino acids in length, five amino acids in length, six amino acids in length, seven amino acids in length, eight amino acids in length, nine amino acids in length, ten amino acids in length, eleven amino acids in length, twelve amino acids in length, thirteen amino acids in length, fourteen amino acids in length, fifteen amino acids in length, sixteen amino acids in length, seventeen amino acids in length, eighteen amino acids in length, nineteen amino acids in length, or twenty amino acids in length. In some embodiments, a linker peptide sequence may be up to fifty amino acids in length. One skilled in the art will recognize that alternative linker sequences exist (functional or not), and may be incorporated into constructs as described herein.

### Signal Peptide

In some embodiments, a coding sequence comprises one or more nucleic acid sequences that each encode a signal peptide.

In some embodiments, a signal peptide is encoded by a nucleic acid sequence that is at least 85%, at least 90%, at least 91%, at least 92%, at least 93%, at least 94%, at least 95%, at least 96%, at least 97%, at least 98%, or at least 99%, or 100% identical to a sequence included in Table 16.

TABLE 16
Signal Peptide Polynucleotide Sequence

| | | SEQ ID |
|---|---|---|
| Description | Sequence | NO: |
| Pisum | GCTTCTATGATATCCTCTTCAGCTGTGACTACAGTCAGCCGTGCTTCT | 148 |
| sativum | ACGGTGCAATCGGCCGCGGTGGCTCCATTCGGCGGCCTCAAATCCATG | |
| chloroplast | ACTGGATTCCCAGTTAAGAAGGTCAACACTGACATTACTTCCATTACA | |
| ribulose- | AGCAATGGTGGAAGAGTAAAGTGC | |
| 1,5- | | |
| bisphosphate | | |
| carboxylase/ | | |
| oxygenase | | |
| small | | |
| subunit | | |
| signal | | |
| peptide | | |
| (PeaChlSP) | | |
| polynucleotide | | |
| sequence | | |
| A. thaliana | ATGGCTTCCTCTATGCTCTCCTCTGCCGCTGTGGTTACCTCCCCGGCT | 187 |
| ribulose- | CAAGCCACCATGGTCGCTCCATTCACTGGTTTGAAGTCATCCGCTTCT | |
| 1,5- | TTCCCGGTCACCCGCAAGGCCAACAACGACATTACTTCCATCACAAGC | |
| biphosphate | AATGGGGGAAGAGTTAGCTGC | |
| carboxylase | | |
| small | | |
| subunit | | |
| (rbcS) | | |
| Choroplastic | | |
| signal | | |
| peptide | | |

In some embodiments, a signal peptide is or comprises an amino acid sequence that is at least 85%, at least 90%, at least 91%, at least 92%, at least 93%, at least 94%, at least 95%, at least 96%, at least 97%, at least 98%, or at least 99%, or 100% identical to a sequence included in Table 17.

TABLE 17
Signal Peptide Polypeptide Sequence

| | | SEQ |
|---|---|---|
| Description | Sequence | ID NO: |
| PeaChlSP | MASMISSSAVTTVSRASTVQSAAVAPF | 150 |
| signal | GGLKSMTGFPVKKVNTDITSITSNGGR | |
| peptide | VKC | |
| polypeptide | | |
| sequence | | |
| A. thaliana | MASSMLSSAAVVTSPAQATMVAPFTGL | 188 |
| ribulose- | KSSASFPVTRKANNDITSITSNGGRVS | |
| 1,5- | C | |
| biphosphate | | |
| carboxylase | | |
| small | | |
| subunit | | |
| (rbcS) | | |
| Choroplastic | | |
| signal | | |
| peptide | | |

### Exemplary Construct Components

### Promoters

In some embodiments, a construct described herein comprises a promoter. The term “promoter” refers to a DNA sequence recognized by enzymes/polypeptides that can promote and/or initiate transcription of an operably linked gene. For example, a promoter typically refers to a nucleotide sequence to which an RNA polymerase and/or any associated factor binds and from which the process of and/or initiate of transcription can occur. Thus, in some embodiments, a construct comprises one of the non-limiting example promoters described herein operably linked to a coding region.

In some embodiments, a promoter is an inducible promoter. In some embodiments, a promoter is a constitutive promoter. In some embodiments, a promoter is a cell specific promoter. In some embodiments, a promoter is a plant cell specific promoter. In some embodiments, a promoter is a tissue specific promoter. In some embodiments, a promoter is a plant tissue specific promoter. In some embodiments, a promoter is a viral promoter. In some embodiments, a promoter is a chimeric promoter. In some embodiments, a promoter is an engineered promoter.

In some embodiments, a promoter is an inducible promoter, a constitutive promoter, a plant cell promoter, a viral promoter, a chimeric promoter, an engineered promoter, a tissue-specific promoter, or any other type of promoter known in the art.

In some embodiments, a promoter may comprise an additional regulatory region such as an enhancer and/or a 5′ UTR. In some embodiments, a promoter may be but is not limited to: 2×CaMV 35S, 2×CaMV 35S+5′UTR TMV, AtAct2, AtSUC2, H4, H4 (S. lycopersicum)+5′UTR, LHB1B1, LHB1B1 (A. thaliana)+5′UTR, Nos, Nos+5′UTR TMV, ocs, ocs (A. tumefaciens)+5′UTR, OsActin+5′UTR, PvUbi1+3, PvUbi1+3 promoter, PvUbi2, PvUbi2_mut, RbcS2B, RolC, rrEaActBlast2, rrEaAs2Blast1, rrEaDPA4Blast1, rrEaH3Blast2, rrEaUbiBlast1, RsS1, RTBV, ZmUbi, or any combination thereof.

In some embodiments, a promoter may be but is not limited to: a 2×CaMV 35S+TMV 5′UTR promoter, an AthAct2 promoter, an AthLHB1B1 promoter, a BdEF1a promoter, a BdUbi10 promoter, a CaMV 35S long promoter, a CaMV 35S short promoter, a CsVMV promoter, a Mas promoter, a Nos promoter, an Ocs promoter, an OsActin promoter, a PsSEOF1 promoter, a PvUbi1+3 promoter, a PvUbi2 promoter, a RolC promoter, an RSs1 promoter, an RTBV promoter, an rrEaAct1Blast promoter, an rrEaActBlast2 promoter, an rrEaAs2Blast1 promoter, an rrEaCons1 promoter, an rrEaCons2 promoter, an rrEaCons3 promoter, an rrEaCons4 promoter, an rrEaCons5 promoter, an rrEaCons6 promoter, an rrEaCons7 promoter, an rrEaCons8 promoter, an rrEaDPA4Blast1 promoter, an rrEaH3Blast2 promoter, an rrEaLeaf1 promoter, an rrEaLeaf4 promoter, an rrEaUbiBlast1 promoter, a prom7_V1343 promoter, a prom7_V2197 promoter, a prom7_V956 promoter, a SlHis4 promoter, or a ZmUbi promoter.

In some embodiments, a promoter is one listed herein as included in Table 3. In some embodiments, a promoter sequence is at least 85%, at least 90%, at least 95%, at least 98% or at least 99% identical to a sequence included in Table 3. In some embodiments, a promoter is a characteristic portion of a sequence included in Table 3.

TABLE 3
Exemplary Promoter Polynucleotide Sequences

| Description | Sequence | SEQ ID NO: |
|---|---|---|
| Exemplary | CTGCAGTGCAGCGTGACCCGGTCGTGCCCCTCTCTAGAGATAATGAGC | 26 |
| Zea mays | ATTGCATGTCTAAGTTATAAAAAATTACCACATATTTTTTTTGTCACA | |
| Ubiquitin 1 | CTTGTTTGAAGTGCAGTTTATCTATCTTTATACATATATTTAAACTTT | |
| promoter | ACTCTACGAATAATATAATCTATAGTACTACAATAATATCAGTGTTTT | |
| (ZmUbi1) | AGAGAATCATATAAATGAACAGTTAGACATGGTCTAAAGGACAATTGA | |
| | GTATTTTGACAACAGGACTCTACAGTTTTATCTTTTTAGTGTGCATGT | |
| | GTTCTCCTTTTTTTTTGCAAATAGCTTCACCTATATAATACTTCATCC | |
| | ATTTTATTAGTACATCCATTTAGGGTTTAGGGTTAATGGTTTTTATAG | |
| | ACTAATTTTTTTAGTACATCTATTTTATTCTATTTTAGCCTCTAAATT | |
| | AAGAAAACTAAAACTCTATTTTAGTTTTTTTATTTAATAATTTAGATA | |
| | TAAAATAGAATAAAATAAAGTGACTAAAAATTAAACAAATACCCTTTA | |
| | AGAAATTAAAAAAACTAAGGAAACATTTTTCTTGTTTCGAGTAGATAA | |
| | TGCCAGCCTGTTAAACGCCGTCGACGAGTCTAACGGACACCAACCAGC | |
| | GAACCAGCAGCGTCGCGTCGGGCCAAGCGAAGCAGACGGCACGGCATC | |
| | TCTGTCGCTGCCTCTGGACCCCTCTCGAGAGTTCCGCTCCACCGTTGG | |
| | ACTTGCTCCGCTGTCGGCATCCAGAAATTGCGTGGCGGAGCGGCAGAC | |
| | GTGAGCCGGCACGGCAGGCGGCCTCCTCCTCCTCTCACGGCACCGGCA | |
| | GCTACGGGGGATTCCTTTCCCACCGCTCCTTCGCTTTCCCTTCCTCGC | |
| | CCGCCGTAATAAATAGACACCCCCTCCACACCCTCTTTCCCCAACCTC | |
| | GTGTTGTTCGGAGCGCACACACACACAACCAGATCTCCCCCAAATCCA | |
| | CCCGTCGGCACCTCCGCTTCAAGGTACGCCGCTCGTCCTCCCCCCCCC | |
| | CCCTCTCTACCTTCTCTAGATCGGCGTTCCGGTCCATGGTTAGGGCCC | |
| | GGTAGTTCTACTTCTGTTCATGTTTGTGTTAGATCCGTGTTTGTGTTA | |
| | GATCCGTGCTGCTAGCGTTCGTACACGGATGCGACCTGTACGTCAGAC | |
| | ACGTTCTGATTGCTAACTTGCCAGTGTTTCTCTTTGGGGAATCCTGGG | |
| | ATGGCTCTAGCCGTTCCGCAGACGGGATCGATTTCATGATTTTTTTTG | |
| | TTTCGTTGCATAGGGTTTGGTTTGCCCTTTTCCTTTATTTCAATATAT | |
| | GCCGTGCACTTGTTTGTCGGGTCATCTTTTCATGCTTTTTTTTGTCTT | |
| | GGTTGTGATGATGTGGTCTGGTTGGGCGGTCGTTCTAGATCGGAGTAG | |
| | AATTCTGTTTCAAACTACCTGGTGGATTTATTAATTTTGGATCTGTAT | |
| | GTGTGTGCCATACATATTCATAGTTACGAATTGAAGATGATGGATGGA | |
| | AATATCGATCTAGGATAGGTATACATGTTGATGCGGGTTTTACTGATG | |
| | CATATACAGAGATGCTTTTTGTTCGCTTGGTTGTGATGATGTGGTGTG | |
| | GTTGGGCGGTCGTTCATTCGTTCTAGATCGGAGTAGAATACTGTTTCA | |
| | AACTACCTGGTGTATTTATTAATTTTGGAACTGTATGTGTGTGTCATA | |
| | CATCTTCATAGTTACGAGTTTAAGATGGATGGAAATATCGATCTAGGA | |
| | TAGGTATACATGTTGATGTGGGTTTTACTGATGCATATACATGATGGC | |
| | ATATGCAGCATCTATTCATATGCTCTAACCTTGAGTACCTATCTATTA | |
| | TAATAAACAAGTATGTTTTATAATTATTTTGATCTTGATATACTTGGA | |
| | TGATGGCATATGCAGCAGCTATATGTGGATTTTTTTAGCCCTGCCTTC | |
| | ATACGCTATTTATTTGCTTGGTACTGTTTCTTTTGTCGATGCTCACCC | |
| | TGTTGTTTGGTGTTACTTCTGCAG | |
| Exemplary | TCGAGGTCATTCATATGCTTGAGAAGAGAGTCGGGATAGTCCAAAATA | 27 |
| Oryza sativa | AAACAAAGGTAAGATTACCTGGTCAAAAGTGAAAACATCAGTTAAAAG | |
| Actin 1 | GTGGTATAAAGTAAAATATCGGTAATAAAAGGTGGCCCAAAGTGAAAT | |
| promoter | TTACTCTTTTCTACTATTATAAAAATTGAGGATGTTTTTGTCGGTACT | |
| (OsAc1) | TTGATACGTCATTTTTGTATGAATTGGTTTTTAAGTTTATTCGCTTTT | |
| | GGAAATGCATATCTGTATTTGAGTCGGGTTTTAAGTTCGTTTGCTTTT | |
| | GTAAATACAGAGGGATTTGTATAAGAAATATCTTTAAAAAAACCCATA | |
| | TGCTAATTTGACATAATTTTTGAGAAAAATATATATTCAGGCGAATTC | |
| | TCACAATGAACAATAATAAGATTAAAATAGCTTTCCCCCGTTGCAGCG | |
| | CATGGGTATTTTTTCTAGTAAAAATAAAAGATAAACTTAGACTCAAAA | |
| | CATTTACAAAAACAACCCCTAAAGTTCCTAAAGCCCAAAGTGCTATCC | |
| | ACGATCCATAGCAAGCCCAGCCCAACCCAACCCAACCCAACCCACCCC | |
| | AGTCCAGCCAACTGGACAATAGTCTCCACACCCCCCCACTATCACCGT | |
| | GAGTTGTCCGCACGCACCGCACGTCTCGCAGCCAAAAAAAAAAAAAGA | |
| | AAGAAAAAAAAGAAAAAGAAAAAACAGCAGGTGGGTCCGGGTCGTGGG | |
| | GGCCGGAAACGCGAGGAGGATCGCGAGCCAGCGACGAGGCCGGCCCTC | |
| | CCTCCGCTTCCAAAGAAACGCCCCCCATCGCCACTATATACATACCCC | |
| | CCCCTCTCCTCCCATCCCCCCAACCCTACCACCACCACCACCACCACC | |
| | TCCACCTCCTCCCCCCTCGCTGCCGGACGACGAGCTCCTCCCCCCTCC | |
| | CCCTCCGCCGCCGCCGCGCCGGTAACCACCCCGCCCCTCTCCTCTTTC | |
| | TTTCTCCGTTTTTTTTTTCCGTCTCGCTCTCGATCTTTGGCCTTGGTA | |
| | GTTTGGGTGGGCGAGAGGCGGCTTCGTGCGCGCCCAGATCGGTGCGCG | |
| | GGAGGGGGGGGATCTCGCGGCTGGGGCTCTCGCCGGCGTGGATCCGGC | |
| | CCGGATCTCGCGGGGAATGGGGCTCTCGGATGTAGATCTGCGATCCGC | |
| | CGTTGTTGGGGGAGATGATGGGGGGTTTAAAATTTCCGCCATGCTAAA | |
| | CAAGATCAGGAAGAGGGGAAAAGGGCACTATGGTTTATATTTTTATAT | |
| | ATTTCTGCTGCTTCGTCAGGCTTAGATGTGCTAGATCTTTCTTTCTTC | |
| | TTTTTGTGGGTAGAATTTGAATCCCTCAGCATTGTTCATCGGTAGTTT | |
| | TTCTTTTCATGATTTGTGACAAATGCAGCCTCGTGCGGAGCTTTTTTG | |
| | TAGGTAGA | |
| Exemplary | GAAGCCAACTAAACAAGACCATAACCATGGTGACATTTGACATAGTTG | 28 |
| Panicum | TTTACTACTTGCTTGAGCCCCACCCTTGCTTATCGGTTGAACATTACA | |
| virgatum L. | AGATACACTGCGGGTGGCCTAAGGCACACCGTCCGAAACCGGCAAACC | |
| Ubiquitin 2 | AAGCCTGATCGCCGAAATCCAAAATCACTACCGGCAATCTCTAAAGTT | |
| promoter | TATTTCATCCTTATATGACGAGGAAAGAAAAGAAGAGAGAAATAATAT | |
| (PvUbi2) | CTTAACTTCTAAATCAGTCGCGTCAACTTTCTCGGCTAAGAAAGTGAG | |
| | CACTATCATTTCGCAGACCATGTCATGAGTGCCGACTTGCCATATCTT | |
| | ATTATATTCTTATTTATTTAATTATAATCCCATTGCAATACGTCTATT | |
| | CTATCATGGCCTGCCACTAACGCTCCGTCTAACGTCGTTAAGCCATTG | |
| | TCATAAGCGGCTGCTCAAAACTCTTCCCGGTGGAGGCGAGGCGTTAAC | |
| | GGCGTCTACAAATCTAACGGCCACCAACCATCCAGCCGCCTCTCGAAA | |
| | GCTCCGCTCCGATCGCGGAAATTGCGTGGCGGAGACGAGCGGGCTCCT | |
| | CTCACACGGCCCGGAACCGTCACGGCACGGGTGGGGGATTCCTTCCCC | |
| | AACCCTCCCCACCTCTCCTCCCCCCGTCGCAGCCCATAAATACAGGGC | |
| | CCTCCGCGCCTCTTCCCACAATCTCACATCGTCTCATCGTTCGGAGCG | |
| | CACAACCCCCGGGTTCCAAATCCAAATTGCTCTTCTCGCGACCCTCGG | |
| | CGATCCTTCCCCCGCTTCAAGGTACGGCGATCGTCTCCCCCGTCCTCT | |
| | TGCCCCATCTCCTCGCTCGGCGTGGTTTGGTGGTTCTGCTTGGTCTGT | |
| | GGCTAGGAACTAGGCTGAGGCGTTGACGAAATCATGCTAGATCCGCGT | |
| | GTTTCCTGATCGTGGGTGGCTGGGAGGTGGGGTTTTCGTGTAGATCTG | |
| | ATCGGTTCCGCTGTTTATCCTGTCATGCTCATGTGATTTGTGGGGATT | |
| | TTAGGTCGTTTGTCCGGGAATCGTGGGGTTGCTTCTAGGCTGTTCGTA | |
| | GATGAGATCGTTCTCACGATCTGCTGGGTCGCTGCCTAGGTTCAGCTA | |
| | GGTCTGCCCTGTTTTTGGGTTCGTTTTCGGGATCTGTACGTGCATCTA | |
| | TTATCTGGTTCGATGGTGCTAGCTAGGAACAAACAACTGATTCGTCCG | |
| | ATCGATTGTTTTGTTGCCATGTGCAAGGTTAGGTCGTTATCTGATTGC | |
| | TGTAGATCAGAGTAGAATAAGATCATCACAAGCTAGCTCTTGGGCTTA | |
| | TTATGAATCTGCGTTTGTTGCATGATTAAGATGATTATGCTTTTTCTT | |
| | ATGCTGCCGTTTGTATATGATGCGGTAGCTTTTAACTGAATAGCACAC | |
| | CTTTCCTGTTTAGTTAGATTAGATTAGATTGCATGATAGATGAGGATA | |
| | TATGCTGCTACATCAGTTTGATGATTCTCTGGTACCTCATAATCAACT | |
| | AGCTCATGTGCTTAAATTGAAACTGCATGTGCCACATGATTAAGATGC | |
| | TAAGATTGGTGAAGATATATACGCTGCTGTTCCTATAGGATCCTGTAG | |
| | CTTTTACCTGGTCAACATGCATCGTCCTGTTATGGATAGATATGCATG | |
| | ATAGATGAAGATATGTACTGCTACAATTTGATGATTCTTTTGTGCACC | |
| | TGATGATCATGCATGCTCTTTGCCCTTACTTTGATATACTTGGATGAT | |
| | GGCATGCTTAGTACTAATGATGTGATGAACACACATGACCTGTTGGTA | |
| | TGAATATGATGTTGCTGTTTGCTTGTGATGAGTTCTGTTTGTTTACTG | |
| | CTAGGCACTTACCCTGTTGTCTGGTTCTCTTTTGCAG | |
| Exemplary | CCACTGGAGAGGGGCACACACGTCAGTGTTTGGTTTCCACTAGCACGA | 29 |
| Panicum | GTAGCGCAATCAGAAAATTTTCAATGCATGAAGTACTAAACGAAGTTT | |
| virgatum L. | ATTTAGAAATTTTTTTAAGAAATGAGTGTAATTTTTTGCGACGAATTT | |
| Ubiquitin 1 | AATGACAATAATTAATCGATGATTGCCTACAGTAATGCTACAGTAACC | |
| fusion | AACCTCTAATCATGCGTCGAATGCGTCATTAGATTCGTCTCGCAAAAT | |
| promoter | AGCACAAGAATTATGAAATTAATTTTACAAACTATTTTTATTTAATAC | |
| (PvUbi1+3) | TAATAATTAACTGTCAAAGTTTGTGCTACTCGCAAGAGTAGCGCGAAC | |
| | CAAACACGGCCTGGAGGAGCACGGTAACGGCGTCGACAAACTAACGGC | |
| | CACCACCCGCCAACGCAAAGGAGACGGATGAGAGTTGACTTCTTGACG | |
| | GTTCTCCACCCCTCTGTCTCTCTGTCACTGGGCCCTGGGTCCCCCTCT | |
| | CGAAAGTTCCTCTGGCCGAAATTGCGCGGCGGAGACGAGGCGGGCGGA | |
| | ACCGTCACGGCAGAGGATTCCTTCCCCACCCTGCCTGGCCCGGCCATA | |
| | TATAAACAGCCACCGCCCCTCCCCGTTCCCCATCGCGTCTCGTCTCGT | |
| | GTTGTTCCCAGAACACAACCAAAATCCAAATCCTCCTCCTCCTCCCGA | |
| | GCCTCGTCGATCCCTCACCCGCTTCAAGGTACGGCGATCCTCCTCTCC | |
| | CTTCTCCCCTCGATCGATTATGCGTGTTCCGTTTCCGTTTCCGATCGA | |
| | GCGAATCGATGGTTAGGACCCATGGGGGACCCATGGGGTGTCGTGTGG | |
| | TGGTCTGGTTTGATCCGCGATATTTCTCCGTTCGTAGTGTAGATCTGA | |
| | TCGAATCCCTGGTGAAATCGTTGATCGTGCTATTCGTGTGAGGGTTCT | |
| | TAGGTTTGGAGTTGTGGAGGTAGTTCTGATCGGTTTGTAGGTGAGATT | |
| | TTCCCCATGATTTTGCTTGGCTCGTTTGTCTTGGTTAGATTAGATCTG | |
| | CCCGCATTTTGTTCGATATTTCTGATGCAGATATGATGAATAATTTCG | |
| | TCCTTGTATCCCGCGTCCGTATGTGTATTAAGTTTGCAGGTGCTAGTT | |
| | AGGTTTTTCCTACTGATTTGTCTTATCCATTCTGTTTAGCTTGCAAGG | |
| | TTTGGTAATGGTCCGGCATGTTTGTCTCTATAGATTAGAGTAGAATAA | |
| | GATTATCTCAACAAGCTGTTGGCTTATCAATTTTGGATCTGCATGTGT | |
| | TTCGCATCTATATCTTTGCAATTAAGATGGTAGATGGACATATGCTCC | |
| | TGTTGAGTTGATGTTGTACCTTTTACCTGAGGTCTGAGGAACATGCAT | |
| | CCTCCTGCTACTTTGTGCTTATACAGATCATCAAGATTATGCAGCTAA | |
| | TATTCGATCAGTTTCTAGTATCTACATGGTAAACTTGCATGCACTTGC | |
| | TACTTATTTTTGATATACTTGGATGATAACATATGCTGCTGGTTGATT | |
| | CCTACCTACATGATGAACATTTTACAGGCCATTAGTGTCTGTCTGTAT | |
| | GTGTTGTTCCTGTTTGCTTCAGTCTATTTCTGTTTCATTCCTAGTTTA | |
| | TTGGTTCTCTGCTAGATACTTACCCTGCTGGGCTTAGTTATCATCTTA | |
| | TCTCGAATGCATTTTCATGTTTATAGATGAATATACACTCAGATAGGT | |
| | GTAGATGTATGCTACTGTTTCTCTACGTTGCTGTAGGTTTTACCTGTG | |
| | GCAACTGCATACTCCTGTTGCTTCGCTAGATATGTATGTGCTTATATA | |
| | GATTAAGATATGTGTGATGGTTCTTTAGTATATCTGATGATCATGTAT | |
| | GCTCTTTTAACTTCTTGCTACACTTGGTAACATGCTGTGATGCTGTTT | |
| | GTTGATTCTGTAGCACTACCAATGATGACCTTATCTCTCTTTGTATAT | |
| | GATGTTTCTGTTTGTTTGAGGCTTGTGTTACTGCTAGTTACTTACCCT | |
| | GTTGCCTGGCTAATCTTCTGCAGATGCAGATC | |
| Exemplary | GAATTCGGATCTTCGAAGGTAGGCTGCAGTTCTTGAATTGTTGAATTA | 30 |
| Oryza sativa | TTATTATCTTCATCTTCATTCATCTGTAACTACTGATTCATCTGGTTT | |
| Cytochrome | GTTATTACCGATCGTAATGCCGTTGTTTTGTCAAAAAAAAAAAAGGAG | |
| c gene | ATCGGTTTGTTATTACCGATCATAATGCTGTTCTTTTATAAAAAAAAA | |
| promoter | ACATGGATCTATTGGCATAATCTTTTTGCGCCAGGTACTCCGACCATT | |
| (OsCc1) | ACTCGGTTACCGACGAAAGCCGGTGAGATTTGGATAAACTTCGCCAAA | |
| | AATTTAAATTTCCGTTTGATCTCTCAAACGTGGGCTGGTTTAGGCCTG | |
| | TTTAATGTTTAGACACATGTATGGAGTACTAAATATTAATAAAAAAAA | |
| | TAATTACACAGATCGTGTGTAAATTGCGAGATAAATCTTTTAAGCCTA | |
| | ATTGCTCCATGAACAATGTGGTGTTACAGTAAACATTTGCTAATGACA | |
| | GATTAATTAGGCTTAATAAATTCGTCTCACAGTTTACAGGTGAAATAT | |
| | GTAATTTATTTATTATTAAGTCTATATATAATACTTTAAATACGTGAC | |
| | CGTATATCCCGATGGGAGACACGTAAAACTTTTTAACCAAGTTCTAAA | |
| | CACAACCTTGCTTCACAGTTTCTTGATCTCTATGGGTAGGGGTGGGCA | |
| | GAAAAAGACCGAACCGAAAGACCGAACCGAAAAGGCCGAGACCGAGAC | |
| | CGAAAAGATCGAGACCGAGAAATTCGGTCCTAGGTAATGAAAGACCGA | |
| | ATTTTGTTCGGTCAATTTGGTTAGTTTTCTCGGGTAACCGAATAGACC | |
| | GAAAAGACCAAATTATCAGAAAATATCTAAATACAATCTACAACCCAC | |
| | TATGTTTAATAGGATTAAACTCTAATTTTTTACATCCCTACTTCTTTT | |
| | AGGCATGCAACCTAATAAGAGTCTTTACTCATAAGTGCTTACGAAATT | |
| | TTTTTGTGATTTTTGTGTTGAAAATTTCCATTATTTCTTTGCATATAT | |
| | GAAAATGTTGTTGAATTTCGGTCAGGACCGAGACCGAGACTGAATTTG | |
| | TCAGTCCTAACATTTTTTCACCGAAATTCAGTCTTCACTTTTCAAAGA | |
| | CTGAAAAGACCGAAAGACTGAAGACCGAGACCGAAATTTTCGGTTAGA | |
| | CCGAATGCCCACCCCTATCTACGGGCTTGATAAGATCAATAACCGTAA | |
| | TTACCGAAGCGGTTGCGTGACTTGCTGTTGCATTTGTCAACCCTAACA | |
| | TAGTACTACCTCCGTTTCAAGGTTCCGTTTCAGAGTTTGTAAAACTTT | |
| | CCTAGTATTAACCCATGTTTTAACTTGCAACGGGAGGAAGTTAACATC | |
| | CTATACGCCTGAAATCCCTTTAAAAAAAAAGAACATTTATACGCTGGA | |
| | ACCGATTCTGAACCGGTCCGTCCACCCACCGACCCACCAACGGTGCGA | |
| | TTTCCACCGTCCACCAAACGCGAGCCGCCTCCACCCTCCACCTATCGA | |
| | GTCAAAGACGACGACTCTACCAGAGCACGTGGACCCGGTCCACGAACG | |
| | GAACGCCCTTACACCGAATGGGCCGTTGGGTGTCCACGCCTCCCACAC | |
| | CCACACCCCCCTTGCCTTTTTCTGCAAGACACGGAAACCTTCTGGAAC | |
| | CGCGTGGATTCCCCGAAACGCCCCTGCCCCCACGCTCCACCCGTTCAA | |
| | TAATTCTAGGGGTATTATCGTAGTTTCGCCACCTGCCCTTCCGCCGCG | |
| | CTGGTGTATACTAGGGCACGCGCTCCTCGGAATCGCCACGAGCCCACG | |
| | AGCCAGAAAAAAAAGGAAAAAAAGAGAGTCGTAGTTCGCCTCTTCTTC | |
| | CTCCTCTCGTTCTCGCGGCGGCGGCGGAG | |
| Exemplary | ACAGAGTAATCCTTCAAGACACATAATAACTCACGAATGTAAAGAACT | 31 |
| Epipremnum | ACAAACACACAAAATTGTTCAAAAAAATTTATGCAAGAAATTTTTTAA | |
| Aureum | GTTACATTATAGCACATTCACATAAGTGAGTGTCAAATTGATGGATAA | |
| Ubiquitin | TCTCCTATATTTTATAAAAAATTACACTCACATGAGTACATGTTATAA | |
| promoter | TCTAATAAGAAATCATTATAGTATATAAATTATTTCTCATGTTTATGA | |
| (rrEaUbi1) | TAGCACGCACCACTTGCAACACGTAAAGTATGTACGTGACTACATGTA | |
| | CAAATCTAAATAATGTTGGGGTAAGATAAAAATTTAACAAATTTAACA | |
| | TGTAAATACTTTTGGGTCAGACTTAATGCATCGTTTAAGAAAAGCGAT | |
| | GCTGGATCGCACACCCATGATCAAATAATTTCTTGTAAATATCTTTTT | |
| | GAAAAATTTTAAGTTAATTAAATATACTCCCGTTAAAATATTTTTTTA | |
| | TAAAAAATCTGCTACATAAATGTCATTTATATCCCCATTGCATATGTA | |
| | TATATACATATATATACCATATATGCTGGTTATATATAAAGAGATATA | |
| | TTTTTAACAAAGTAATTATTTTTAACTGACAGTTATTGGTCTGGGGCA | |
| | AATTTAATTTAACAGGGTATATATGCAATTTACCCAAAACTTTTTAAT | |
| | CTTTTCCCGTGGGGCGAAGGAGCAGACCGGCTCCGATCCAAACATTCG | |
| | CCCTCGTATTCCGTCTCCTCAATCTCTCTCTCTCTCTCTCTCTTTCTT | |
| | CGCTCCCTCCTGCAAGCAAAAGCCAATATTTTTCTTCCTCCAAATCCC | |
| | CCTTTCCTCTACAAACAACACCCCTCACTGCTTCTCTTGCTTCTCTCC | |
| | CCGCCTCAGAATCACCAGATCGCAACTCGATCTAGGGTTTAGAACCGG | |
| | TACGTCTCC | |
| Exemplary | GGGGTGCGACAACATTACCTAGTTCATTAGTGGGACCATCTGCAGATT | 32 |
| Epipremnum | GAGGACTCTTGGATCATCCGAAAGTAGTTCCAGTGCCTTGACTCAGAC | |
| Aureum | TTATTAGAGTAACACTAGAGCGGCACCGACCATTTCTCGACGGGATCG | |
| Ubiquitin | AGTTCTTTCCAGTTAGGAGGAGTTGGTGGAGACACTAAAAATAGGGTT | |
| promoter | CGTTTTGACCCTGGGTGGGTCTGCAACAGACGAGAATGTGCGAAAATG | |
| (rrEaUbi3) | ACAATGACATCACTTTAATTTGGAGACGAGTAGTGGGCCCAGTAAGAA | |
| | TTTTGTGGTGCCATCATTATTAAGCATGTTAAGGTTGGGAGTCTTTTG | |
| | ATACCTTATTGGGCTTATTTGGGCTTAGTTTTATTTTTTTTTTCTTCA | |
| | TATTTTTTATATGATTTTCATGCATTTTTTTATGTGTGAGGAATATTT | |
| | TGGTCATAAAATGTCTTTTACAGTTAGAGTTATGAGAGAGTTTATAAA | |
| | TATGTTCTATAACTCTCTTTTTTAATTATTGGAAAATCTTGTTGCGAA | |
| | TTTTGAGTATTTTATTGTACTCTATGAGAGAGGTTGAGAGGACCGCTA | |
| | CTTACGGTCATCCGCGAGAGACGGGGACTTACATTCCTCATCGCCCAC | |
| | CCCTTTGCTGCCTTTGTGACTGTGTTCCTCGTTAAGAAGTCTGATCCC | |
| | TGAAAAGTTGCTAAAGATACCTCTATCACATCTGACGTGTTGTGAGGA | |
| | TCGTAATGGTGTAATCACAACTCAAATCAGATGTCGGACGGGCTTGAT | |
| | TTCATACTGGTAGATTCTTTTGGAACCCGTGATTGCACAACGTATGGC | |
| | TGGGGGGGTACGTGTCGTCGTGGCACTATGTAAGGCAAGCTGAAGTGA | |
| | GCATAAACAACAAGTAGACCTCGATGGATGAGTTTGTCATCTTCAGGC | |
| | ATTCATCAATGTGGACGC | |
| Exemplary | GCAAGTTGCGTAATCGTGCTCCGTTGCTGAGTGGTTTGTTTTGGACTC | 33 |
| Epipremnum | CTGGTTCTGGCTCGTCAGACAACTGGTAAACATAGAAATAATCAACTA | |
| Aureum | AGCTGCAAATTTCCCGCAAGGGAAGTTGGCGGCAGACAATTGAACTGT | |
| Ubiquitin | AACATTTGAATGTAATGGTTTTTCGGTTGTTGACAGGATAATTTTAGT | |
| promoter | TAACACCCCGGCTCTCTCACCCGGAGTTCCTGCCTGTGCCTTGCGGGC | |
| (rrEaUbi4) | ATTGGGCTTTTGAACTGTGTTTGGACTCATGGAATTGCATGAAAACTT | |
| | GGAGCGTGAGGTTGCACGTTAGAAGTGTATAGAAGTGCCTTAGGAGTT | |
| | AGCTCCGGGTGTGGGA | |
| Exemplary | TCTGTTGTGACATGTGACGTGAATCTAAAGAAACACTCGCTATTTGCA | 34 |
| Epipremnum | TTATTTTTCTTGTATTTTCAGTGAAGCAAAGTGTCAAAGTTGCCTATC | |
| Aureum | GTTGGTCAAGATCCTGGATCTGTTGGGGATCTCTCCTTACATTGCAAT | |
| Actin | TTCCTCTTGTCCTTATTGTTTTAATTTCGGAAAGCGCTATTTGTTGCT | |
| promoter | TGCTTTGTTGCAGTTTACATCATCCCTTCTTGATGCTCTTTGGGGGGA | |
| (rrEaAct1) | AATCTCTCTGGGACATTCGATAATATTTGGAAAAAAATAGTCTGCGAG | |
| | CCAGAAGCCCCAGTGCGCTCTCGTTTGTTTTTCGTCTCATGCTTCTTA | |
| | ATCTTGTATTTGGCATTTGGGAAGAGTGACACAGGATATGCTATCTAA | |
| | TTAGTAAATGAATGTGTTTATCGTGCGGACAACTAATTATTCAGATGG | |
| | ATGAAATTCTTGAAGATTTATGTTAAGAATAAATCATTATGCAATAAT | |
| | TTCCTAAATGTCAATTGATATTGCATCGGATTTCACATGCACCAGTAA | |
| | AACTAGTACTTACCTGTGGTTCATGACAAACACGATTTTTTTTAATTT | |
| | TTCTAATGCAATTTACTTTTTCTGCTCATACTTTCTCTTAAAGTAACA | |
| | TCCATCTCCACTTGTTTTTTTTTCCTTTCTCAAATATATCTTGATCCA | |
| | CACTTACCGACAAGCCTGTACTGGTTTATCTGATTGTTAAATTTGATG | |
| | TTACATTTGAATGGGAAGAGATATCATGTTAGTTCGGTTCTAGCATTA | |
| | AAATGCCTAGTACATCTTACTCCTTTTGCAGAATGACTTTCTTTATAC | |
| | ATATGGTACGTTATTTTTCTTGAAATGGAGCTTGCCCAAGCAGAATTT | |
| | CTTTTTTCATGGATGATGGTTGTCGTTGGTAGTTTAATTTTATCATTA | |
| | ACCTTTCACGTCTTACATATTTCTCAGATATTGGTGAATATTTTAATC | |
| | TGAAACGTAAAGTGAGCAGGTGTAGA | |
| Exemplary | ACACCATCACCCTCATTGGTTTCTGTAGCATGACTCTGAGCTACGATG | 35 |
| Epipremnum | GAAGATCCAAGTTCCAAAATAAAAATAGTCCCTGGTGTCACTATTGGG | |
| Aureum | TCGCTCAAGCAAGGCATATATTGTCTAAGTTGACCTGAAAATTGCATG | |
| Actin | ACCAAATCTGATTCCCGCTCACGGCCCTGTCCGCGACGTCACTCGTGA | |
| promoter | AACTCCCTATTAGAGGGAGAGTGGAGCATCATGCTTGGAAGCTAAAAA | |
| (rrEaAct2) | AAAATGGATGATGTCAAAATTCCAAACTAACAATAAGTAATGAGCTGT | |
| | ATTGGGCAAATAATACTAATATAGAAGTAGTAAGTAAAAGAGAGAGAA | |
| | AAAAGAGTCAATAAAAAAAATGCAACAAAAGGTTTTGTGCTTACCGAC | |
| | CGCTGTCCGTGGCACTTCCCGGTTCGTGGGGGACATTTGTTGGCAAAT | |
| | ATCTTTTTTATTATTATTCAAAAAAAATGAAAAGGAAGGGAGATAAGA | |
| | AAAGACAAGAGACTGCTCTCCCACACCTTAATGCAACTCAGGTTGGTT | |
| | CACTTATGGTGCAACACAAGGTAACCTGCAATCAAAAGGTCTGGGCAG | |
| | CTGGATTTTGTGCTGTCTTACTTTAGAAGCACAACTCTTTGACATATG | |
| | CTTTGGTGGAATTTATCAAAGGAAAAGCTCCTGATGTTGTAAACAGTG | |
| | GGTCAATAACACAACAGGCTAAAACAGATTTCATGAAAAATTCATTCT | |
| | CTGGTCTGCTATAGAAAAGTTCTTCACAGTGATTTTGGGGCTACCAGA | |
| | TGTTCAGAGGTGGTATTCAGCTAGCGGCAATTTCAAGCTGGGTTGCAG | |
| | TTTGAAGGCAGAAAAGAGACAGGCTGTTCTTTGCCTGATCAGGGATTG | |
| | TCCCCCATCTCTCTCCCTCTGTCTTTTCTCTCCCTCCTGCACTCCCAT | |
| | CAGAAAATAGCAGGGAGAGAGAGACTGATGGGTCTTTCCCTCTCTCAC | |
| | TGATTTTTCCCTTTCTCCTGGTTTTCTCT | |
| Exemplary | ATGGCTGCATTACCTGACGTACAATATTATTGGTAGGTAATTCGAGAT | 36 |
| Epipremnum | TAACTATGAAATATGTATATGTGTCTCACAACTAAGTAATGGCCAACT | |
| Aureum | TAGTTAACCAGGTTATGAACAAGTTAAAGTTGGTGTCAAACTCTGGAT | |
| Histone H3 | TAACTTCAGAGTAACCACTCTCTACTTAGAACCCAAAACTTATGTAAG | |
| promoter | TTAATACTAATGAGTAATCTCTGGACTAACCCACCACACCAATTCATG | |
| (rrEaH32) | ACTTTTGGAAGAAAGATTACTTATTAATCCGAATAATTTGGACCCCCT | |
| | TTTTGAAAATAATTATTGAGTTAATTCTGAACTATTAAATATTTCATA | |
| | TTATTAATAATCATTTTAAATAAAAGCTGCTGATCTTAGTTGTAATTT | |
| | TTTTTACTATTAACAAAGAGAGAGATAAACGCATTTTTTTCTATTTTT | |
| | ATACCAAAATTAACCCATATTCAAATTTTGGGGATGACACATGAATTA | |
| | AGCTAGTTTCTCATTAGAAAAAGATCTTAGCCTTACTTATTAGGGGTA | |
| | CATAGATAATTTAATTTTTTTAAATGTTTTCACGTAATTTCAAACCAT | |
| | TTAGGCCAAAGCGGGCCGAATTCAAATTCGTGGGCTCGGTGTCACGTT | |
| | GGTCCAGCCAGAGCAGTGTTATCAGCTTCCTACCTGGTGAAGGTACGC | |
| | CATTGGCTGTTGTCCGACGACGCGGATCAAGTTGCATAAACAAATTCG | |
| | CACCGTCCGATGAAAGCGAATGATCCCGATTCACTCAAGGGGCCCCCG | |
| | CTGCGGCAGCGGCGGAGAAAATTTCGAACTCTCCGCCAAAAGGGCTCC | |
| | TCTCTCTCTCTCTCTCTACAAATACTCGCCAAAGGCTCCCCCTTTGTT | |
| | CTACCCAAGCAGTCCTCGCTGCTCCAGATCGAGAGGCATCCAGAGAGC | |
| | GTCCGAAAGAA | |
| Exemplary | TGTTACAAAACAGAAGAAATTTGACATATGTGTTGAACATAATCTTGT | 37 |
| Epipremnum | CCTAATATTTTTTTATTTTTTTTAAAATTTTAAAGTACTTAAAAATAT | |
| Aureum | TATCTCTTAAAATCAACGTCCATCACACAATTTGTAAATTTGGACCAA | |
| Histone H3 | GTCAACCTGAGTTGATTGACTTAGTTCATATTCAATTATTTAGTATAT | |
| promoter | ACGATTCAATACAAATTATTTAAATAATAATATAATATTTAAAATATA | |
| (rrEaH31) | ATTTACATATTTTATAAAAATTAAAAATAATAAAAATTTAAATATGTG | |
| | ACTTAATAAGTCACAAGAGTTTTGATATGTGGATAAAAGTTTCTATAG | |
| | ACAAACAAGATTTTTTTGAATAAAAATTATCTACTAAATTGTAAAAGT | |
| | TTTATGAGATTTTAAGATTTGTTATTTATAAACATAAAATTTTTAATG | |
| | TTAAATAAAATAAAATAATTGATGAAAATTTAAATTATCCTATTATAT | |
| | TGTCAAAAAATTCACAAGAGAAGAGTGGCAGTCAAAAGTTATCCTCGA | |
| | ATTATTTTCTTAATATAGATAAAAAAAAGATCTCGAGAGAATTTAAAA | |
| | TTTAGAAACCCCTGGCCCACCCTAGCCCAGAAAGCTCGCCAGCCGCGC | |
| | TGGCCGGGCCCGCACTTACGCTCCCAAGAGGGAGCTTGGCCAAGGTCG | |
| | AAAGTGACGGCGATCGCGATCCGCGTGCTATTCCTCAGGATCATCTCA | |
| | ACCGTTCTTTGAGACAAATCGACGATCTCGACTAACCACCGAGAAATT | |
| | CAAAAGTTCCAAAACCGGCTCCCGCCTTTCGTGCGCCTACAAGTATCC | |
| | ATCCCTTCCCTCAGGGCTTGAATCGTCTCCACCCCTCCGAACACAAAG | |
| | CATTTCCTCCTGCTGCACCGAAACCCTAGGCCCTCGTTC | |
| Exemplary | GTCAACATGGTGGAGCACGACACTCTGGTCTACTCCAAAAATGTCAAA | 38 |
| Cauliflower | GATACAGTCTCAGAAGATCAAAGGGCTATTGAGACTTTTCAACAAAGG | |
| Mosaic | ATAATTTCGGGAAACCTCCTCGGATTCCATTGCCCAGCTATCTGTCAC | |
| virus | TTCATCGAAAGGACAGTAGAAAAGGAAGGTGGCTCCTACAAATGCCAT | |
| promoter | CATTGCGATAAAGGAAAGGCTATCATTCAAGATCTCTCTGCCGACAGT | |
| (2x | GGTCCCAAAGATGGACCCCCACCCACGAGGAGCATCGTGGAAAAAGAA | |
| CaMV35S) | GAGGTTCCAACCACGTCTACAAAGCAAGTGGATTGATGTGATAACATG | |
| | GTGGAGCACGACACTCTGGTCTACTCCAAAAATGTCAAAGATACAGTC | |
| | TCAGAAGATCAAAGGGCTATTGAGACTTTTCAACAAAGGATAATTTCG | |
| | GGAAACCTCCTCGGATTCCATTGCCCAGCTATCTGTCACTTCATCGAA | |
| | AGGACAGTAGAAAAGGAAGGTGGCTCCTACAAATGCCATCATTGCGAT | |
| | AAAGGAAAGGCTATCATTCAAGATCTCTCTGCCGACAGTGGTCCCAAA | |
| | GATGGACCCCCACCCACGAGGAGCATCGTGGAAAAAGAAGAGGTTCCA | |
| | ACCACGTCTACAAAGCAAGTGGATTGATGTGACATCTCCACTGACGTA | |
| | AGGGATGACGCACAATCCCACTATCCTTCGCAAGACCCTTCCTCTATA | |
| | TAAGGAAGTTCATTTCATTTGGAGAGGACA | |
| Exemplary | GTCAACATGGTGGAGCACGACACTCTGGTCTACTCCAAAAATGTCAAA | 164 |
| Cauliflower | GATACAGTCTCAGAAGATCAAAGGGCTATTGAGACTTTTCAACAAAGG | |
| Mosaic | ATAATTTCGGGAAACCTCCTCGGATTCCATTGCCCAGCTATCTGTCAC | |
| virus | TTCATCGAAAGGACAGTAGAAAAGGAAGGTGGCTCCTACAAATGCCAT | |
| promoter | CATTGCGATAAAGGAAAGGCTATCATTCAAGATCTCTCTGCCGACAGT | |
| (2x | GGTCCCAAAGATGGACCCCCACCCACGAGGAGCATCGTGGAAAAAGAA | |
| CaMV35S) | GAGGTTCCAACCACGTCTACAAAGCAAGTGGATTGATGTGATAACATG | |
| (2) | GTGGAGCACGACACTCTGGTCTACTCCAAAAATGTCAAAGATACAGTC | |
| | TCAGAAGATCAAAGGGCTATTGAGACTTTTCAACAAAGGATAATTTCG | |
| | GGAAACCTCCTCGGATTCCATTGCCCAGCTATCTGTCACTTCATCGAA | |
| | AGGACAGTAGAAAAGGAAGGTGGCTCCTACAAATGCCATCATTGCGAT | |
| | AAAGGAAAGGCTATCATTCAAGATCTCTCTGCCGACAGTGGTCCCAAA | |
| | GATGGACCCCCACCCACGAGGAGCATCGTGGAAAAAGAAGAGGTTCCA | |
| | ACCACGTCTACAAAGCAAGTGGATTGATGTGACATCTCCACTGACGTA | |
| | AGGGATGACGCACAATCCCACTATCCTTCGCAAGACCCTTCCTCTATA | |
| | TAAGGAAGTTCATTTCATTTGGAGAGGACACGCTCGAGTATAAGAGCT | |
| | CATTTTTACAACAATTACCAACAACAACAAACAACAAACAACATTACA | |
| | ATTACATTTACAATTATCGATAC | |
| Exemplary | GAACCGCAACGTTGAAGGAGCCACTCAGCCGCGGGTTTCTGGAGTTTA | 39 |
| Agrobacterium | ATGAGCTAAGCACATACGTCAGAAACCATTATTGCGCGTTCAAAAGTC | |
| tumefaciens | GCCTAAGGTCACTATCAGCTAGCAAATATTTCTTGTCAAAAATGCTCC | |
| Nopaline | ACTGACGTTCCATAAATTCCCCTCGGTATCCAATTA | |
| synthase gene | | |
| promoter | | |
| (NOS) | | |
| Exemplary | AGCGGAGAATTAAGGGAGTCACGTTATGACCCCCGCCGATGACGCGGG | 165 |
| Agrobacterium | ACAAGCCGTTTTACGTTTGGAACTGACAGAACCGCAACGTTGAAGGAG | |
| tumefaciens | CCACTCAGCCGCGGGTTTCTGGAGTTTAATGAGCTAAGCACATACGTC | |
| Nopaline | AGAAACCATTATTGCGCGTTCAAAAGTCGCCTAAGGTCACTATCAGCT | |
| synthase | AGCAAATATTTCTTGTCAAAAATGCTCCACTGACGTTCCATAAATTCC | |
| gene promoter | CCTCGGTATCCAATTAGAGTCTCATATTCACTCTCA | |
| (NOS) (2) | | |
| Exemplary | CTGAAAGCGACGTTGGATGTTAACATCTACAAATTGCCTTTTCTTATC | 40 |
| Agrobacterium | GACCATGTACGTAAGCGCTTACGTTTTTGGTGGACCCTTGAGGAAACT | |
| tumefaciens | GGTAGCTGTTGTGGGCCTGTGCTCTCAAGATGGATCATTAATTTCCAC | |
| Octopine | CTTCACCTACGATGGGGGGCATCGCACCGGTGAGTAATATTGTACGGC | |
| synthase | TAAGAGCGAATTTGGCCTGTAAGATCCTTTTTACCGACAACTCATCCA | |
| gene promoter | CATTGATGGTAGGCAGAAAGTTAAAGGATTATCGCAAGTCAATACTTG | |
| (Ocs) | CCCATTCATTGATCTATTTAAAGGTGTGGCCTCAAGGATAATCGCCAA | |
| | ACCATTATATTTGCAATCTACCA | |
| Exemplary | ATTTTTCAAATCAGTGCGCAAGACGTGACGTAAGTATCCGAGTCAGTT | 41 |
| Agrobacterium | TTTATTTTTCTACTAATTTGGTCGTTTATTTCGGCGTGTAGGACATGG | |
| tumefaciens | CAACCGGGCCTGAATTTCGCGGGTATTCTGTTTCTATTCCAACTTTTT | |
| Mannopine | CTTGATCCGCAGCCATTAACGACTTTTGAATAGATACGCTGACACGCC | |
| synthase | AAGCCTCGCTAGTCAAAAGTGTACCAAACAACGCTTTACAGCAAGAAC | |
| gene promoter | GGAATGCGCGTGACGCTCGCGGTGACGCCATTTCGCCTTTTCAGAAAT | |
| (Mas) | GGATAAATAGCCTTGCTTCCTATTATATCTTCCCAAATTACCAATACA | |
| | TTACACTAGCATCTGAATTTCATAACCAATCTCGATACACCAAATCG | |
| Exemplary | CCAGAAGGTAATTATCCAAGATGTAGCATCAAGAATCCAATGTTTACG | 42 |
| Cassava | GGAAAAACTATGGAAGTATTATGTAAGCTCAGCAAGAAGCAGATCAAT | |
| Vein | ATGCGGCACATATGCAACCTATGTTCAAAAATGAAGAATGTACAGATA | |
| Mosaic | CAAGATCCTATACTGCCAGAATACGAAGAAGAATACGTAGAAATTGAA | |
| Virus | AAAGAAGAACCAGGCGAAGAAAAGAATCTTGATGACGTAAGCACTGAC | |
| promoter | GACAACAATGAAAAGAAGAAGATAAGGTCGGTGATTGTGAAAGAGACA | |
| (CsCMV) | TAGAGGACACATGTAAGGTGGAAAATGTAAGGGCGGAAAGTAACCTTA | |
| | TCACAAAGGAATCTTATCCCCCACTACTTATCCTTTTATATTTTTCCG | |
| | TGTCATTTTTGCCCTTGAGTTTTCCTATATAAGGAACCAAGTTCGGCA | |
| | TTTGTGAAAACAAGAAAAAATTTGGTGTAAGCTATTTTCTTTGAAGTA | |
| | CTGAGGATACAACTTCAGAGAAATTTGTAAGTTTGT | |
| Exemplary | AGGAGTCGACAAAATTTAGAACGAACTTAATTATGATCTCAAATACAT | 43 |
| Arabidopsis | TGATACATATCTCATCTAGATCTAGGTTATCATTATGTAAGAAAGTTT | |
| thaliana | TGACGAATATGGCACGACAAAATGGCTAGACTCGATGTAATTGGTATC | |
| Actin 2 | TCAACTCAACATTATACTTATACCAAACATTAGTTAGACAAAATTTAA | |
| promoter | ACAACTATTTTTTATGTATGCAAGAGTCAGCATATGTATAATTGATTC | |
| (AthAct2) | AGAATCGTTTTGACGAGTTCGGATGTAGTAGTAGCCATTATTTAATGT | |
| | ACATACTAATCGTGAATAGTGAATATGATGAAACATTGTATCTTATTG | |
| | TATAAATATCCATAAACACATCATGAAAGACACTTTCTTTCACGGTCT | |
| | GAATTAATTATGATACAATTCTAATAGAAAACGAATTAAATTACGTTG | |
| | AATTGTATGAAATCTAATTGAACAAGCCAACCACGACGACGACTAACG | |
| | TTGCCTGGATTGACTCGGTTTAAGTTAACCACTAAAAAAACGGAGCTG | |
| | TCATGTAACACGCGGATCGAGCAGGTCACAGTCATGAAGCCATCAAAG | |
| | CAAAAGAACTAATCCAAGGGCTGAGATGATTAATTAGTTTAAAAATTA | |
| | GTTAACACGAGGGAAAAGGCTGTCTGACAGCCAGGTCACGTTATCTTT | |
| | ACCTGTGGTCGAAATGATTCGTGTCTGTCGATTTTAATTATTTTTTTG | |
| | AAAGGCCGAAAATAAAGTTGTAAGAGATAAACCCGCCTATATAAATTC | |
| | ATATATTTTCCTCTCCGCTTTGAATACTGTATTTTTACAACAATTACC | |
| | AACAACAACAAACAACAAACAACATTACAATTACTATTTACAATTAC | |
| Exemplary | AGGAGTCGACAAAATTTAGAACGAACTTAATTATGATCTCAAATACAT | 166 |
| Arabidopsis | TGATACATATCTCATCTAGATCTAGGTTATCATTATGTAAGAAAGTTT | |
| thaliana | TGACGAATATGGCACGACAAAATGGCTAGACTCGATGTAATTGGTATC | |
| Actin 2 | TCAACTCAACATTATACTTATACCAAACATTAGTTAGACAAAATTTAA | |
| promoter | ACAACTATTTTTTATGTATGCAAGAGTCAGCATATGTATAATTGATTC | |
| (AthAct2) | AGAATCGTTTTGACGAGTTCGGATGTAGTAGTAGCCATTATTTAATGT | |
| (2) | ACATACTAATCGTGAATAGTGAATATGATGAAACATTGTATCTTATTG | |
| | TATAAATATCCATAAACACATCATGAAAGACACTTTCTTTCACGGTCT | |
| | GAATTAATTATGATACAATTCTAATAGAAAACGAATTAAATTACGTTG | |
| | AATTGTATGAAATCTAATTGAACAAGCCAACCACGACGACGACTAACG | |
| | TTGCCTGGATTGACTCGGTTTAAGTTAACCACTAAAAAAACGGAGCTG | |
| | TCATGTAACACGCGGATCGAGCAGGTCACAGTCATGAAGCCATCAAAG | |
| | CAAAAGAACTAATCCAAGGGCTGAGATGATTAATTAGTTTAAAAATTA | |
| | GTTAACACGAGGGAAAAGGCTGTCTGACAGCCAGGTCACGTTATCTTT | |
| | ACCTGTGGTCGAAATGATTCGTGTCTGTCGATTTTAATTATTTTTTTG | |
| | AAAGGCCGAAAATAAAGTTGTAAGAGATAAACCCGCCTATATAAATTC | |
| | ATATATTTTCCTCTCCGCTTTGAA | |
| Exemplary | AGGAGAATATCATTTTTAAGTAAAATTTTGAATTCAAATGTTACGTGT | 44 |
| Solanum | ATTATTTAATTCATCAATTTGCCTTGTCATAGCGAGTACATTACAAAC | |
| lycopersicum | ATCACATATATTTGATTGATTGTCAAAAAATATCAAAATATATATCAA | |
| Histone | TTTTAAGAGGTATAGGTGTCTAATATGTACTAGCCCTAATTTAAATAT | |
| H4 | CTAAATTAATTATTCGGATGAATCTATATACCATCTTTTTAATGGACA | |
| promoter | CCCAAAATCACACATCAAACATCATATACATGTTGAAAACATATTATT | |
| (SIHis4) | GATATAGCTACATATATGTTTTAATATAAATAAAAGACGAGTCATATA | |
| | TTCAAAAATTAAGAATCAAATAATTTTAATTTATTTAATATTCAAAAC | |
| | TTAATACTATTTAAATTTAGATATTCTAATTTTAATACACGTCTGATA | |
| | AAATAGATGAGGACTAAATAAATAATTTGAGACTATCTTTTCTTTATT | |
| | TGGCGGCCCACAAATAATTTAGATTCTCGTAACCCCCTCTTTTTCTCT | |
| | CACTGAAAAAGCACAATCCGTGTCCAAACACAAAGAAGCACTCGACAC | |
| | CGTAGATCTCCATTCAGATCAACGGCTTATATTCAGTTTTCTCCATTC | |
| | ACGTGGATCGACATTCTTATCCGTCCGATTATCAATAAATTTCCCAAA | |
| | ATTTAGCGGCCATGATTTTAACCCCGCCTCATTTCAAACCGCCCACGA | |
| | AATCCTCGACGCCCAAATTCACCAACTATAAATAGCCACCACCATCCC | |
| | CTTCATCAATCATCAAATTTCATAACCCTAGAATCATCACCTTTTTCA | |
| | AATTTC | |
| Exemplary | AGGAGATATGACTGGTAAGTTTTTCTTGCCAATACGAATTAGAAAACA | 45 |
| Arabidopsis | TGTCTTTGAAGATGAACTGTATTTTTTTTTTTTACTTTGTTGTCATTT | |
| thaliana | TAATGTACTTTCTTATCAGGATTAAATCTTCTGTAATTTAGAGTAGTT | |
| Light- | TTTTTAACAAGATAATTAACAAACTTAGAGTAATGAAAATTGAGATGT | |
| harvesting | TCAGTTTTCACTCATATTTCACATTTTGGTGAAAGAGTGGGTAGTATG | |
| chlorophyll- | CAACGTTCTAAGTATGTTTGGACTTTGTATCATGTTGTTTTGATTCTT | |
| protein | TGACGACATGTCTATTTGGGAAACACCAATGACGTGTACCTTGAGACT | |
| complex II | GATACGATTCAAAGGGATAGAAACACGTCAGATTTACAAGTGGCACCT | |
| subunit B1 | CTTCAATGGACAATGGGTATTCCAATATGCTAAGATGCTACGAGATAT | |
| Promoter | CTAATTTATCTAACACAACTCAATTCCAAACCAAAAATCTGATGCCAG | |
| (AthLHB1B1) | CTCGACAAGACAAAAAATCTAAGCTCAAAAATGTCAACAACCAATAGA | |
| | AATCAAGGCATTGACGATATCACGAGATAAGCAAATTAAATCTTCAAG | |
| | TTTTGCAATTCATATGTACGTTATAAATACCCAAAAACCTCACCGTAA | |
| | CCTAGCTATCCAATTTCATCACATCTTATTAACTAAAGAGCCTTTTAC | |
| | TTGCGCCACACTCTCACCGC | |
| Exemplary | ACCTCAACCTTCGCTCACAGTGAAGGCTTGAAACTCGCTTTTTAACAT | 46 |
| Epipremnum | TGTAAGTGGGCTGATTTTGAACTCATCTCATCGTAAATCTTTAAGCTT | |
| aureum | TGACTTCCCACGATGTTGTCCAGTCTATTAGATTTTTTATGGTTTTTT | |
| ribulose | TTTCTTTTTTCGCTGAAAGTTCCTACTTAAAATAGTCACCCACTAGGT | |
| bisphosphate | ACAGAAGAGTCAGCTACATGAAAAATACCTTAATATAGAAAAACGTAT | |
| carboxylase/ | TTATTGTATTAAAATTTGAACCCTCCCCACTTAAAATGATGCGTACCA | |
| oxygenase | CTTAGACCTAGTTGAGATTTATTGTTGCACCTGGGAGAGAGTTGAATA | |
| activase 2 | GGGTCCGGATTCCCACTTAGTTTCTCTGGAATCTAGATAGGGCGGTCA | |
| promoter | GCTTTATCTTAATTAGTGACAAGGCACTAGTTGGAGTTAGTTTTTATA | |
| (rrEaCons1) | TTGAACATACTCTTAAACTTTTAGTTCCCTATTTTGAGAGAAAGTATT | |
| | TGAAGTAATTTTAAACTTTTGGTTAAATCTTCCACTTTTGACCAAAAG | |
| | TTCAAAATTAAAGTTTCCCAAGTTCAAGAAAGAATGGTATCATTAGCC | |
| | CATATAAGAACTAAATTAAAATCAGTTTGATTCATTCTTATTAAGCTC | |
| | CAACATACTCAACAGCACAACCAACAGCATGACTTGTGTAAACTGAAA | |
| | AACTCAGAGAGAGAGAGATAGAGACTCTGAACGAGTGGTGCTGAGCAG | |
| | CAGTGGCTGCTTCATGAAGAGTTTGGCGTGACGACAAAACCATCAAAA | |
| | ACACAGAAGAGGAATTTCATTGCCGACAATCACCATGTCTCTGTAATA | |
| | CTGCTGGTCCTGATGAAATGCTTGAAGGAAAAAAAACTGGCATTAAAG | |
| | AGGAGGGGAAAAAACCGAAAATTTTAGTGGAGTCGGGAAGCCCGGGAA | |
| | CCCGAACCATTCCTGGCGTCTGACGTCCTCCGCTGCCGAGAGGATGCT | |
| | GTAGCTGATGGGCCCCACTTCCCCACACTCCCCAACTTCCAACGTCAG | |
| | GACACGACTCTATCTGCGCAGAAGCAACCAACCCTGATGCGCCACGTG | |
| | TCGCCCCACCCCAATCCGCAGTGTGTGGCCGTTGTGGCCCTCGCGATC | |
| | CAATCCACAGGATGCTTCACTCTCCTCCTCTCCTCCGCAAGCCAAACG | |
| | GGAAAATAACGGAGCAGGGCAGACTCCAGAGCCTCCGCAGGCCGCTTT | |
| | ATATATAACTCGCCCTCCCACGCCTCCTACGGTCATCACTGCCGCGAG | |
| | GAGCTTTGCTTTTGGTGGACGCGGCGATCTCCCCCCATCTCCTTCTCG | |
| | GTCTTCC | |
| Exemplary | CCTCAACCTTCGCTCACAGTGAAGGCTTGAAACTCGCTTTTTAACATT | 167 |
| Epipremnum | GTAAGTGGGCTGATTTTGAACTCATCTCATCGTAAATCTTTAAGCTTT | |
| aureum | GACTTCCCACGATGTTGTCCAGTCTATTAGATTTTTTATGGTTTTTTT | |
| ribulose | TTCTTTTTTCGCTGAAAGTTCCTACTTAAAATAGTCACCCACTAGGTA | |
| bisphosphate | CAGAAGAGTCAGCTACATGAAAAATACCTTAATATAGAAAAACGTATT | |
| carboxylase/ | TATTGTATTAAAATTTGAACCCTCCCCACTTAAAATGATGCGTACCAC | |
| oxygenase | TTAGACCTAGTTGAGATTTATTGTTGCACCTGGGAGAGAGTTGAATAG | |
| activase 2 | GGTCCGGATTCCCACTTAGTTTCTCTGGAATCTAGATAGGGCGGTCAG | |
| promoter | CTTTATCTTAATTAGTGACAAGGCACTAGTTGGAGTTAGTTTTTATAT | |
| (rrEaCons1) | TGAACATACTCTTAAACTTTTAGTTCCCTATTTTGAGAGAAAGTATTT | |
| (2) | GAAGTAATTTTAAACTTTTGGTTAAATCTTCCACTTTTGACCAAAAGT | |
| | TCAAAATTAAAGTTTCCCAAGTTCAAGAAAGAATGGTATCATTAGCCC | |
| | ATATAAGAACTAAATTAAAATCAGTTTGATTCATTCTTATTAAGCTCC | |
| | AACATACTCAACAGCACAACCAACAGCATGACTTGTGTAAACTGAAAA | |
| | ACTCAGAGAGAGAGAGATAGAGACTCTGAACGAGTGGTGCTGAGCAGC | |
| | AGTGGCTGCTTCATGAAGAGTTTGGCGTGACGACAAAACCATCAAAAA | |
| | CACAGAAGAGGAATTTCATTGCCGACAATCACCATGTCTCTGTAATAC | |
| | TGCTGGTCCTGATGAAATGCTTGAAGGAAAAAAAACTGGCATTAAAGA | |
| | GGAGGGGAAAAAACCGAAAATTTTAGTGGAGTCGGGAAGCCCGGGAAC | |
| | CCGAACCATTCCTGGCGTCTGACGTCCTCCGCTGCCGAGAGGATGCTG | |
| | TAGCTGATGGGCCCCACTTCCCCACACTCCCCAACTTCCAACGTCAGG | |
| | ACACGACTCTATCTGCGCAGAAGCAACCAACCCTGATGCGCCACGTGT | |
| | CGCCCCACCCCAATCCGCAGTGTGTGGCCGTTGTGGCCCTCGCGATCC | |
| | AATCCACAGGATGCTTCACTCTCCTCCTCTCCTCCGCAAGCCAAACGG | |
| | GAAAATAACGGAGCAGGGCAGACTCCAGAGCCTCCGCAGGCCGCTTTA | |
| | TATATAACTCGCCCTCCCACGCCTCCTACGGTCATCACTGCCGCGAGG | |
| | AGCTTTGCTTTTGGTGGACGCGGCGATCTCCCCCCATCTCCTTCTCGG | |
| | TCTT | |
| Exemplary | AGGAACAAGTGCCACCTGAGCCAAGGCGCTCATTGGCGTCTTGATAGT | 47 |
| Epipremnum | TTCTTTTATGGTATACATGCTGTTGTAAGAATCTTAATGTTTTAAATT | |
| aureum | TGCATCTGCATGTATATATCCACGTTTTGGTGTAATATCCACGTCTAT | |
| Metallothio | ACCCTTGTGAAAGGTATCTGTATGCATCCAAGTATAGTTAAATCACTT | |
| nein-like | TTTAAAATTTACAGCTATGTCCCTTGTAAAGCTATAATGACATTTTTG | |
| protein type | TGCATCTAGAAAGAGTACTCACTCGGGGACTCTTCTAACAGACAAGCA | |
| 3 promoter | CATGATGAGAAATTTGCACCCGCACAATTCAAATTTGATTCTGAAAGA | |
| (rrEaCons2) | CTTGCAACTTACAAACTATCTTAAGTACGTACGACCACAAATTATCTC | |
| | AAGTGTACTCTTTGTTCCACAAATAACTTTTACATTGACACTATTTAA | |
| | GGACGACACTGATCAGAGATAAAATGACAAAATGAAAGGGGACTCATC | |
| | TAAGTTAGACAAATCCCGAAACTTATTTCATATACCCTAAGAACACTT | |
| | GCCCCCCTAATTAACGACGGTACATGAGTAACATGTTTGCTTTTCACA | |
| | TGAATACAAATGGCAGTACATATATGTAAGCTAGCAAGAAGGATATGT | |
| | GGGTGATAATTATCTGTATATGGTCCGTATCCACCTCCCTCTCTAGTA | |
| | TCTCCATCACGTAGCCAGAGGTCATCGGATTTGTACACCAGTTGCATG | |
| | TGCCTGTGCATCTGTTGCCAGTTGCGTGTGACAGTGCAGCTGTGTATT | |
| | GCCACAAAAAAAAAAGGAATAAAAAGGTAGTGCAACTGGGTAACGGTG | |
| | CAAGGATAGCCGTGTCTGCCCATCTGAACCCAAAAGGGCGACGACGAC | |
| | GACTCGGGGAGGTGAAAGAAGAGGAACTGGCGTGAGAGCTGGTGGGGC | |
| | AGCCCCCCTCCTCTCCACCATAATTGAGATTCCTTTGGAAGCTTCCCC | |
| | CATGGAGGCGTGTGCCCGTCACACACAGGAGGCAGAAGCCCTTCCCCT | |
| | CCATCTCTCCTTGTGCCGTGTGCGGCTGCCCATCCAACCCCTGGGGCC | |
| | TATAAATATCGTCGCAGGGGCAGAAGCCCCTCCAGCATAGCTGAAGCT | |
| | TGAGTAGTTCAGAGATATAGCTCTCTTTGATCTCCAGAGAGGCTCCCT | |
| | CCTGACATCACCACC | |
| Exemplary | GTTCCACTCGAGGCAGGAAAAATCTCTGGATTTGGACACTTAACCGAC | 48 |
| Epipremnum | CCCCATTAACACCCCACCTCACATCAGAGCACGGTTTGCCCACTCAAC | |
| aureum | TTGTCAGGCAAACCACATCTTATCTCAAAAGCTATGAGTTACAACGTC | |
| abscisic | AGATAACTAATTTAAATAATAATATAAATTTAAAATATAAATTATATT | |
| stress- | TTTTATTAAATTAAAAGAATAATATTTTTTAAATATCTAATTTTATCC | |
| ripening | AATCAAATTCAAGTTCAACTGATCTATATTAAATAAAAAAATTAATAC | |
| protein 2- | GAATCCAAATTTTAAGTTGACAAATAAATGAATTTTGAATAAAAGAAT | |
| like | CACAAATAAAAAATTACGTTTTCTTGGCGTATATCACCATGCTTGTCT | |
| promoter | TCGTTTAAGAGATTTAAGCAATCATGGACGTCTGCTTATCCACGGATG | |
| (rrEaCons3) | TGAAATATTAAATGATAAAATACTATATTATCTTATATTATAGAAAAA | |
| | TAAATTTTAAATGAGAAGTGGGTATTTATTATGTTTTCATTCAACATA | |
| | CGTGCGAAAGTTTTATCTAGATAGATTAGCGTTAGCATCACTCAAGAA | |
| | TTTTTTTTATTTTCTTAACTGCTTCAAAAAAAGAAATATAAAGGGATT | |
| | GGCCCACGTTAATTAGCTAGAAAAAGTGGGATTGAAACGGGTGTTATC | |
| | CACTTCACATTCTGTGAGCGAATCCGATGCGTGAAGCCCCGCCATCCT | |
| | GACCCGACCGCTGTTCCCCCCTACCCACGAAGAAGCCGTCTGTCCGTC | |
| | TCTTCAATCTCTATACTTCCCCTTCGCCTGCTGCGTACACTCCCGTGG | |
| | CTATAAATAACCACCACAGCCTCTCTGATTTCTTCGTACCCATTACTG | |
| | CAACACCTCTACAGCTACTAGCCGTGTCGCCCGCCCCCCCTTAAGGTC | |
| | ATTCTACCACTGCCAGT | |
| Exemplary | GTTCCACTCGAGGCAGGAAAAATCTCTGGATTTGGACACTTAACCGAC | 168 |
| Epipremnum | CCCCATTAACACCCCACCTCACATCAGAGCACGGTTTGCCCACTCAAC | |
| aureum | TTGTCAGGCAAACCACATCTTATCTCAAAAGCTATGAGTTACAACGTC | |
| abscisic | AGATAACTAATTTAAATAATAATATAAATTTAAAATATAAATTATATT | |
| stress- | TTTTATTAAATTAAAAGAATAATATTTTTTAAATATCTAATTTTATCC | |
| ripening | AATCAAATTCAAGTTCAACTGATCTATATTAAATAAAAAAATTAATAC | |
| protein 2- | GAATCCAAATTTTAAGTTGACAAATAAATGAATTTTGAATAAAAGAAT | |
| like | CACAAATAAAAAATTACGTTTTCTTGGCGTATATCACCATGCTTGTGT | |
| promoter | TCGTTTAAGAGATTTAAGCAATCATGGACGTCTGCTTATCCACGGATG | |
| (rrEaCons3) | TGAAATATTAAATGATAAAATACTATATTATCTTATATTATAGAAAAA | |
| (2) | TAAATTTTAAATGAGAAGTGGGTATTTATTATGTTTTCATTCAACATA | |
| | CGTGCGAAAGTTTTATCTAGATAGATTAGCGTTAGCATCACTCAAGAA | |
| | TTTTTTTTATTTTCTTAACTGCTTCAAAAAAAGAAATATAAAGGGATT | |
| | GGCCCACGTTAATTAGCTAGAAAAAGTGGGATTGAAACGGGTGTTATC | |
| | CACTTCACATTCTGTGAGCGAATCCGATGCGTGAAGCCCCGCCATCCT | |
| | GACCCGACCGCTGTTCCCCCCTACCCACGAAGAAGCCGTCTGTCCGTC | |
| | TCTTCAATCTCTATACTTCCCCTTCGCCTGCTGCGTACACTCCCGTGG | |
| | CTATAAATAACCACCACAGCCTCTCTGATTTCTTCGTACCCATTACTG | |
| | CAACACCTCTACAGCTACTAGCCGTGTCGCCCGCCCCCCCTTAAGGTC | |
| | ATTCTACCACTGCCAGT | |
| Exemplary | GCAACAATGACGCGGATTCAGCCCGCCAAACAGATACCATTAACTCGG | 49 |
| Epipremnum | TTCACTTGTTTAAGAAAGCGTTGTAGATTTTTTTTTAAAATTTATTAA | |
| aureum | TAAAATTTTACCGCCCCCAAAGCCCAAACTAATGTTATCAAGTTGGAA | |
| RNA- | TCTGAAAAAAAAATAGATTCGAGAGAAAGATATTAATTCAATCAAAAT | |
| binding | ACAAATAATTCATGAAAGGTTCTGAATGTATCGTCGATCTTTAATATA | |
| protein | ATTAAATATTAATTGTAAATCATATAAAAACTATTAATTGACTAGTTC | |
| cabeza-like | CAATAGCCAGTCCTTGTCACTCTTGGCTGCATTGCCGGGTATCGGATA | |
| promoter | TTGGCACCGCGGAGAACGCGAGAGGTGCCTCACCGCCAACATGGAAGG | |
| (rrEaCons4) | CGCTTGCGCCTTTCGGTTGACTCCCGAGGTAAACAAGGGGCCAGGGGC | |
| | ATCCACGTAAACACGCCCTCCCCCGGGCCCAGGGGTATCCACGTAAAC | |
| | ACGCCCTTCAGATATGTCTGTGTCGCTTGCGCGGTCCCCGCCCCGCTC | |
| | GTTCCCTTCCCTGTGATAAGCACAAAGCCACGAACCCTGTTCTGGGCC | |
| | TAAACGGGCCACCAAACGATCGGGGGATCCAATCCAGCACGAGTTCCA | |
| | CTGTTCCCTCACCCCATCTAAATCTTAATTTGCTCCAGCTCCACGAGG | |
| | GTACCATTACACAGCTCCCGAAAACGTCCACCAGTTCGCACAGGCTCG | |
| | TCGAGGGGAACACGATAGTGTCTAGTGCGGGGTCCATGGGCCCATCCA | |
| | GTACTGCCGGCCAGTCCACGAAGCCCAACGGGGACCCTGGTTGAACCC | |
| | AAGCGTGGGGTTACAAACGCTCGAG | |
| Exemplary | GCTCCGTCCCTTTTCCCTTTTCTTTCCATTTCTACCATGCGTGTCAGC | 50 |
| Epipremnum | GTGTGCGTCCATTGCTCGAACTGTGTCTGCACGTGTTCATGTGATCAT | |
| aureum | CAGAAGTCTTGTTCGCAGGCCCACCGTTTTCGATTTGGAGATCCCCGG | |
| leaf | ACATAATCCGGAAGAGATCTTCTTTTTTAGCACATGAACATACAGTAA | |
| patterning | TGCGAGAATGGAAGGAGTGAGAAAATATCCTTTGAATCCCGGTTGCAT | |
| promoter | CCCGAATCCTACCGAGAAAGAGAGGATCTCTATCTCAAGCAGTGTAAG | |
| (rrEaAs21) | AAGAGCTCACGGTGGTCTTTCCCGATCATGTCCGGAGGCATGTGATCT | |
| | CAAGTGCTGTGGTGCAAGTAATCCCCTTAGAAGGTTATGATCTCCGTT | |
| | CCGTATCCATCACCGTCTTTCGTACTTCATGGGTTTCTCTTCCCTTCT | |
| | CTCTCCTATCCGTGTATCTTCTCAGATTTGTATGGGAGATACTGTATG | |
| | GGGAGGAGTAGAGTCTGGGTTGTATTCAGTTCCCTCCATTGCCCTTTT | |
| | AGACAAGAGAAAGGAAAAACAGTGAATTCCATGTGTTCTTCTGTCCAA | |
| | CCGTGTCGCCTTGCTGCGAATAGTCCTAGCAATTGCACTGTTGCCATG | |
| | CCTTCCTGTCACTGTAAGATGACACTCTACTCTGTGTGTCTTTTTTGG | |
| | TATTATCTCTAAGGGCAATCCGCACACGTTCCCGTTCATTTACTTCAT | |
| | GTGGAAAAGAAAAAAGTTTGTTTCTTTCTGAAAAAAATCATGGAAGAT | |
| | AATTGTTTTGCCCACTCATTTGCTACTATATATTCTACCTTAATTTGT | |
| | TTGCAACGGGTCAGGTTGTTTAAATCTGACTGTTTAAAGGCTCTATCT | |
| | TTTGGACAGGAATTGATCATATATAAGCAGCCGTGTGTGGTT | |
| Exemplary | CCATCGCTATTCTTGTATTGTCACGAATGCCACCCCTAGATAATTTAT | 51 |
| Epipremnum | TTGTGAAAATATCTTTGAAATACAATTTTTGTGCATAAATTCTCAAAA | |
| aureum | GATGGCATTCATATGAGAATAAGGGTGACAAATGCGTAATGTAACAAT | |
| leaf age | GACATATTTGTAAAAAAAATTCATATCTAATTTTCCAACATTAATCTA | |
| dependent | TCTAAAATATTATAATATCATATCTAATAGATGTTGACCATACGTGAG | |
| promoter | GCATTTGGCACTAGGCCTACCCAAGGAGGATGCAAATGTGTTTTTAAT | |
| (rrEaKan22) | GGAGTTACTTTGCACATCTTTTATACAAGGGGGGCATCGTTACAAAAA | |
| | CTCAAAATTAACTTGTGAGAGGCCGGCTTTATCTTTTTATGGCCCGTA | |
| | AAGCGGAAATATGAGAAGTGGAGAAATGGAATAGGAGACAGGAAGGAA | |
| | GGGATGCACACAAAGCTAAAATGTTAGATCAGAACTTCACTTTTTATC | |
| | AAAAAGAAAATCAGTGGGAAAAAGAATAAAAAAAAAGAATCGAAGCCT | |
| | TCTCTCTCTCTCTCTCTCTCTCTCTCTCTCTCTCTCTCTCTCTCTCCT | |
| | TCTATGTGTGTTTGTCCACACCCCACGTCCACAAAAGAAACATACTCC | |
| | TACTTTCTCCTCTATTTCTCTCTCCTGGCAGCCAAGACCATTCATACC | |
| | GAGTGTCATTTTCCTGCACATACTTCCCCTTCATACAAGAAGTAACCA | |
| | CTTCCACTCTCCCCGTTTCAAGACATTTACCTCCCCTCCAATCCCTCG | |
| | TTCCCCAACTCCCCTCCCAAAACCTTCCTGTTCATCTAGAACACCCAT | |
| | CTGCTCCACACCTCCTACCCTTCCCACACTCCCAACGGGAAGAAGAAC | |
| | TCAGTGTACGAGAAGAAACCCAGAGTCCCGTCTGCGGCGGCGCAGGCG | |
| | GAGGGTAGGGAGGGAGGGAGAGAGAGAGTGAGTGTGTGTGTGTGTGTG | |
| | TGAGAGAGAGAGAGAGAGGT | |
| Exemplary | TGCTCCAATTACATTTGCCATCTGAAAATATATGCCACAGTCTGGTTA | 52 |
| Epipremnum | ATTTTTAAAGAAAAAAAATAATATTCCAGCAGAAGAATGGATCGCTGG | |
| aureum | ATCAAGTTTTTTTTCTGCCCAATTAAAAGTTGAAATGGTGGTCCAAAA | |
| leaf age | TGATTTCTTATTCGGAAAATTGAATATTTTAAAATAATATATATCGTA | |
| dependent | CTGACACGTGAGAATAGCGAAAAGGACGAGCTCACATGAGCCTAACCA | |
| promoter | GATGGTGCATGGTCCCGGTCCAGCTCTCCCTTCCCGTCTTTGCACGGC | |
| (rrEaDPA41 | TCCAATTCCTCTCCCAGCTTTATCTCTTCCATCTCGGTTCCCTTTCAT | |
| | CTCTTCTCCCCAGCTGTAATACGAGAGGAATACCAGTGCAGGTACTCG | |
| | CGCTTCGGCGTCTCTGTCCGCCGCTCCTCCTCCTCACTCCTTCACCAG | |
| | ATCTGTTATAAGCTGAAGCCTCTCAAACCCTAATCTCGAATGTCCCCA | |
| | GGGGTATGAGCCCATCTGCAGCCTTTCCATCCCAGAGATCGATGGGAA | |
| | GCCATCTAATCCTGTAGTTCTGCCTGCTATAGCACTGAGCAGCGGGAG | |
| | AGCAGGCCATGCACCGATCCACCCCTTCGGCTGTATCCTCCTCCTCTT | |
| | CTGATCTCCTCTTCTCCCCCCTCCCTCTCGTTGTGCAAGCAGTTCAGT | |
| | GGGATGCCCGCATCTCTCTCTCTTTCCCCCATATTCTCCCCTCCGCCC | |
| | CCGCTTTCCGTTTCTTTCTCATCTTACAGGTGTAGAGAGAGAGAGAGA | |
| | GAGAGAGAGAGAGAGAGAGCTGTGAGTTAACACAGTAAAAGAAGGCGT | |
| | AGGATTTGCACAGTCGTCGTCTGTCGTCTGAGA | |
| Exemplary | GGAATTCCCACAGAATCAGATTCGGGTACAAATGCGCCAGGAGGAATA | 53 |
| Epipremnum | CACGCCGCCCAAGGTTCCCAAACTACATTATTAATACAAGCCTTAATT | |
| aureum | AGATCAAGTGATCCCGTCAGTGATAAAAATAATAAACAAATAATATGT | |
| salicyclic | TAGGTTTTTTTATTTTTTTATTTTTATAAAAAGAATATTGCATTAAAC | |
| acid stress | CTGTAGTTAATTTATTTATATATAAGCTTTAATGCAACAGAGAGATTT | |
| responsive | GTTGCTAAAATTTTGTAAGGAGCTTAGATTATTATGCCCCTCTTTTTT | |
| promoter | CATAGGGTGAGAGGGGTCCTCCTTGTAGTAGGTTTCTAGAATTCTAAA | |
| (rrEaPR11) | TAGTCACTTAATCAAGTAAATTATAGTTCAAATAAGTGAAATGGATGT | |
| | TTAATTAGGCAAAAATCAGATCTGTAGGACAGAAATTTCTTAATTAGG | |
| | GACATAATTAATTACGATCTTGGCTTTCATAGAACATTATAATATAAA | |
| | TATTTAACTGGGAACCAAAAAAATCTACAAAGGTGTACTTTACACAGA | |
| | CAAATTTCACAATGTTTTTTCAGAATATATAAGATTTTTCTTAGAGAT | |
| | ATAGTAAAGCTCACTTAATAAAAGAGATCACGAGATAAGATCTAGTTG | |
| | ATGATAATAATTATTATAATACTTTATTTAACAAAAATTAAAATAATT | |
| | TTAATTATTATGATAATTATAAAAATATTTATAATAACATCTTTCATA | |
| | AATTAACTCTAAGTTAATTTACACGGTTGTGGTTATGATTATTTAAAA | |
| | ATTAAACAAAGATTAACAAATTTATAATTATAATTAATGAAGTTGTAA | |
| | AATTTAATTAGAATAATCTCAACTACAGTATCAAACAGTCGACGTTGT | |
| | TGGTGGACGTTCCCAGTAGAGAGAAAGAGAGGGAGAGAGAGAGAGGGA | |
| | GGTGGGCGGGGGAAGAGAGAGAAAGCGGAACCCGGACAAACAACTACA | |
| | AAGCTCC | |
| Exemplary | AAGGTATAACGAAGATTTGTTCCGCGTGGAAAAGGCATTAAAAGTGCC | 54 |
| Arabidopsis | ACGTCACTCTCTCTTTTTATTTTATGATTTTCGTATCTCTTCTTCTAC | |
| thaliana | TTGCTTCCCACGTTTCCATCAAGTTTCCGTACATATCTTCTTGTTATC | |
| quick | TGATCCACGCGATCTTTCAACGCGTACTTTTCACGTATTTGTGTTGTC | |
| response | ATGCCTTTGCTGGGATTGTGTTAGATGCTCATTGCTGACGGTAGTTTT | |
| stress | TAGAGAACATTCTAGAAAGAAACTATTTTTCTAACAAAACCACGAACT | |
| responsive | TTGTTTTCTAGTTATTCCACTTTCTAGAATACACCTGACCAAATTAGA | |
| promoter | ATTCTAGAAATGAATTTTAAATAAACCAAAACACCTAAACGAAAAGCA | |
| (rrAtZat12) | AACCATAGGTTTTTGGTTTTAACATATTTCAAATTCATAAAAGTGAAA | |
| | CCAACCTACACCATATTAACCAATATTTATTAGAGTTTTTATATGTTT | |
| | TATGATATTGTTCAAAACTTCAAAAGAGATTTATTCATATAACATACC | |
| | TATACCATACCAATGAATATTAAAATTATGAATTAGTATCCTTATATT | |
| | ATATGAAGTCAATCAAAAAACTTAGAAGCATTTCAAACGGAATCAAAC | |
| | CATTCATATATGAAGTATTATTATTATATCTAGAAGGTGTTGATTTTA | |
| | AACTATTCCGTATAATATATCTAGAAGACGGCTCCGCGCGTGGGGAAT | |
| | GCATCAAACTCAGAGAGTTTAATAGCTTTTTTTGGTTGACGTCAACTA | |
| | CTCAAAAGAGTTTAGTTTTTGATGTGTATATATCCAAATAAAATATCT | |
| | TTAAAAAGAAAATAATAATAATAAATGGTTTCGAGAAAACACGAGGAA | |
| | GATTCTCATCCAACCGAAACGACTCTTTCGTTTTTAGTAGTCTCTTAA | |
| | GCTACGCGGTGTCGCAAATCGTGACCACATAACCCGTTT | |
| Exemplary | GCTACTTCTTTCAGCCACGCACTGCGCTTCAAAACTTCCACGGTACCA | 55 |
| Epipremnum | TAGTCGAGTTTGACGAGAAAATGTCGAACTTGTGGAGAGGAAGAGAAA | |
| aureum | GTGATCCCATGAGAATTCAGAATAAATCCAAGTAGCAGATGAACAGTA | |
| auxin | CTCGTATTGATGCGCTACGTAACGTATAATACCTGGCGAAAACCATAA | |
| signaling | AACCCAAGAGAGCGAATCTTAAGAAGTACTGTTGTTTTTTTTTCTGGG | |
| responsive | GACACGGTGAGAAGAGAAGCCTAGCGTTCTCCCCCAAACAGAGTTCTC | |
| promoter | TCTCCTCCCTCCCCTCCTGTCTAAGTTCTAAAAAGGTGGCGTGGTCGG | |
| (rrEaPin12) | GCACATTGCTTCGTCTCTTGCTTCCCGTTCCTGAACCCATTTAAAGCA | |
| | GGTGTTGCTTTGTTGTCTGCCTACAGAGCTCCACAAAATAGTAAGCAG | |
| | ATACACAACAACACGTACGCCATCGCCATAACTCTCCTTCGCCTCTCC | |
| | CAGTTGCTGGTTACATCTGTTCTACTACGAGCACCTGTCCCCCATTTT | |
| | CTTTCCCTCCTCTCTGCTTTTTCCCTGTTTCGCGCTCTGTCACCGCTT | |
| | CTCCCTTCTCTTTCCCCCTCTGCACTGATGGTTAACGTGCTTAAAATC | |
| | ACTTCAGTTGTCCTCTTCTAATAAGCAGGGTTCTTCATTGAGAAGAAT | |
| | CTCCACAGGTAAGCAAACATCACCTCGTTAGGCTTCTCATTCCACTTC | |
| | TTCACAAAGGGTCCACCGCAAACCCAGATAGCAAGCCCTGCTTCGTCG | |
| | TTTGCCCCTGTTCCATTTCCATTTCCACCCGGGGTCACTCTCAGTCAT | |
| | GGTTTCCCGGGGGAAGCAGTGAGCTGCTTTGTTCTTACTGAAGCCAGG | |
| | CACACAGGGCCTTCCACCACCGCCACCGTTCTCCCTCGTTCCCTGCAT | |
| | CAGAAGAGCCACGTGGTGTTCTTGCAGGAT | |
| Exemplary | AGTAGTAATATTTAATGAGCTTGAAGGAGGATATCAACTCTCTCCAAG | 56 |
| Rice tungro | GTTTATTGGACACCTTTATGCTCATGGTTTTATTAAACAAATAAACTT | |
| bacilliform | CACAACCAAGGTTCCTGAAGGGCTACCGCCAATCATAGCGGAAAAACT | |
| virus | TCAAGACTATAAGTTCCCTGGATCAAATACCGTCTTAATAGAACGAGA | |
| promoter | GATTCCTCGCTGGAACTTCAATGAAATGAAAAGAGAAACACAGATGAG | |
| (RTBV) | GACCAACTTATATATCTTCAAGAATTATCGCTGTTTCTATGGCTATTC | |
| | ACCATTAAGGCCATACGAACCTATAACTCCTGAAGAATTTGGGTTTGA | |
| | TTACTACAGTTGGGAAAATATGGTTGATGAAGATGAAGGAGAAGTTGT | |
| | ATACATCTCCAAGTATACTAAGATTATCAAAGTCACTAAAGAGCATGC | |
| | ATGGGCTTGGCCAGAACATGATGGAGACACAATGTCCTGCACCACATC | |
| | AATAGAAGATGAATGGATCCATCGTATGGACAATGCTTAAAGAAGCTT | |
| | TATCAAAAGCAACTTTAAGTACGAATCAATAAAGAAGGACCAGAAGAT | |
| | ATAAAGCGGGAACATCTTCACATGCTACCACATGGCTAGCATCTTTAC | |
| | TTTAGCATCTCTATTATTGTAAGAGTGTATAATGACCAGTGTGCCCCT | |
| | GGACTCCAGTATATAAGGAGCACCAGAGTAGTGTAATAGATCATCGAT | |
| | CAAGCAAGCGAGAGCTCAAACTTCTAAGAGAGCAA | |
| Exemplary | AAAGTTGGCCCGCTATTGGATTTCGCGAAAGCGGCATTGGCAAACGTG | 57 |
| Agrobacterium | AAGATTGCTGCATTCAAGATACTTTTTCTATTTTCTGGTTAAGATGTA | |
| rhizogenes | AAGTATTGCCACAATCATATTAATTACTAACATTGTATATGTAATATA | |
| promoter | GTGCGGAGATTATCTATGCCAAAATGATGTATTAATAATAGCAATAAT | |
| (RolC) | AATATGTGTTAATCTTTTTCAATCGGGAATACGTTTAAGCGATTATCG | |
| | TGTTGAATAAATTATTCCAAAAGGAAATACATGGTTTTGGAGAACCTG | |
| | CTATAGATATATGCCAAATTTACACTAGTTTAGTGGGTGCAAAACTAT | |
| | TATCTCTGTTTCTGAGTTTAATAAAAAATAAATAAGCAGGGCGAATAG | |
| | CAGTTAGCCTAAGAAGGAATGGTGGCCATGTACGTGCTTTTAAGAGAC | |
| | GCTATAATAAATTGCCAGCTGTGTTGCTTTGGTGCCGACAGGCCTAAC | |
| | GTGGGGTTTAGCTTGACAAAGTAGCGCCTTTCCGCAGCATAAATAAAG | |
| | GTAGGCGGGTGCGTCCCATTATTAAAGGAAAAAGCAAAAGCTGAGATT | |
| | CCATAGACCACAAACCACCATTATTGGAGGACAGAACCTATTCCCTCA | |
| | CGTGGGTCGCTAGCTTTAAACCTAATAAGTAAAAACAATTAAAAGCAG | |
| | GCAGGTGTCCCTTCTATATTCGCACAACGAGGCGACGTGGAGCATCGA | |
| | CAGCCGCATCCATTAATTAATAAATTTGTGGACCTATACCTAACTCAA | |
| | ATATTTTTATTATTTGCTCCAATACGCTAAGAGCTCTGGATTATAAAT | |
| | AGTTTGAATGCTTCGAGTTATGGGTACAAGCAACCTGTTTCCTACTTT | |
| | GTTAAC | |
| Exemplary | CAATCCACCAAATCAAACCGTGAGATTTTTGCAGAGGCAAAACAAGAA | 58 |
| Oryza sativa | AAGCATCTGCTTTATTTCCCTCTTGCTTTCTTTTCATCCCCAACCAGT | |
| sucrose | CCTTTTTTCTTCTGTTTATTTGTAGAAGTCTACCACCTGCAGTCTATT | |
| synthase I | ATTCTACAGAGAAAAAGATTGAACCTTTTTTTCTCCAAAGCTGACAAT | |
| gene | GGTGCCGGCATATGCTAATAGGATACTCCCTTCGTCTAGTCCCTTCGT | |
| promoter | CTAGGAAAAAACCAACCCACTACAATTTTGAATATATATTTATTCAGA | |
| (RSs1) | TTTGTTATGCTTCCTACTCCTTCTCAGTTATGGTGAGATATTTCATAG | |
| | TATAATAAATTTGGACATATATTTGTCCAAATTCATCGCATTATGAAA | |
| | TGTCTCGTTCGATCTAGGTTGTTATATTATGAGACGGAGAGAGTAGAT | |
| | TCGGTTATTTTTGGACAGAGAAAGTACTCGCCTGTGCTAGTGACATGA | |
| | TTAGTGACACCATCAGATTAAAAAAAACATATGTTTTGATTAAAAAAA | |
| | TGGGGAATTTGGGGGGAGCAATAATTTGGGGTTATCCATTGCTGTTTC | |
| | ATCATGTCAGCTGAAAGGCCCTACCACTAAACCAATATCTGTACTATT | |
| | CTACCACCTATCAGAATTCAGAGCACTGGGGTTTTGCAACTATTTATT | |
| | GGTCCTTCTGGATCTCGGAGAAACCCTCCATTCGTTTGCTCGTCTCTG | |
| | ACCACCATTGGGTATGTTGCTTCCATTGCCAAACTGTTCCCTTTTACC | |
| | CATAGGCTGATTGATCTTGGCTGTGTGATTTTTTGCTTGGGTTTTTGA | |
| | GCTGATTCAGCGGCGCTTGCAGCCTCTTGATCGTGGTCTTGGCTCGCC | |
| | CATTTCTTGCGATTCTTTGGTGGGTCGTCAGCTGAATCTTGCAGGAGT | |
| | TTTTGCTGACATGTTCTTGGGTTTACTGCTTTCGGTAAATCTGAACCA | |
| | AGAGGGGGGTTTCTGCTGCAGTTTAGTGGGTTTACTATGAGCGGATTC | |
| | GGGGTTTCGAGGAAAACCGGCAAAAAACCTCAAATCCTCGACCTTTAG | |
| | TTTTGCTGCCACGTTGCTCCGCCCCATTGCAGAGTTCTTTTTGCCCCC | |
| | AAATTTTTTTTTACTTGGTGCAGTAAGAATCGCGCCTCAGTGATTTTC | |
| | TCGACTCGTAGTCCGTTGATACTGTGTCTTGCTTATCACTTGTTCTGC | |
| | TTAATCTTTTTTGCTTCCTGAGGAATGTCTTGGTGCCTGTCGGTGGAT | |
| | GGCGAACCAAAAATGAAGGGTTTTTTTTTTTGAACTGAGAAAAATCTT | |
| | TGGGTTTTTGGTTGGATTCTTTCATGGAGTCGCGACCTTCCGTATTCT | |
| | TCTCTTTGATCTCCCCGCTTGCGGATTCATAATATCCGGAACTTCATG | |
| | TTGGCTCTGCTTAATCTGTAGCCAAATCTTCATATCTCCAGGGATCTT | |
| | TCGCTCTGTCCTATCGGATTTAGGAATTAGGATCTAACTGGTGCTAAT | |
| | ACTAAAGGGTAATTTGGAACCATGCCATTATAATTTTGCAAAGTTTGA | |
| | GATATGCCATCGGTATCTCAATGATACTTACTAAAACCCAACAAATCC | |
| | ATTTGATAAAGCTGGTTCTTTTATCCCTTTGAAAACATTGTCAGAGTA | |
| | TATTGGTTCAGGTTGATTTATTTTGAATCAGTACTCGCACTCTGCTTC | |
| | GTAAACCATAGATGCTTTCAGTTGTGTAGATGAAACAGCTGTTTTTAG | |
| | TTATGTTTTGATCTTCCAATGCTTTTGTGTGATGTTATTAGTGTTGAT | |
| | TTAGCATGGCTTTCCTGTTCAGAGATAGTCTTGCAATGCTTAGTGATG | |
| | GCTGTTGACTAATTATTCTTGTGCAAGTGAGTGGTTTTGGTACGTGTT | |
| | GCTAAGTGTAACCTTTCTTTGCAGTTCCTGAAATTGAGTCATG | |
| Exemplary | AGCTTGCAAAATAGCACACCATTTATGTTTATATTTTCAAATTATTTA | 59 |
| Arabidopsis | TTACATTTCAATATTTCATAAGTGTGATTTTTTTTTTTTTTGTCAATT | |
| thaliana | TCATAAGTGTGATTTGTCATTTGTATTAAACAATTGTATCGCGCAGTA | |
| sucrose-H+ | CAAATAAACAGTGGGAGAGGTGAAAATGCAGTTATAAAACTGTCCAAT | |
| symporter | AATTTACTAACACATTTAAATATCTAAAAAGAGTGTTTCAAAAAAAAT | |
| gene | TCTTTTGAAATAAGAAAAGTGATAGATATTTTTACGCTTTCGTCTGAA | |
| promoter | AATAAAACAATAATAGTTTATTAGAAAAATGTTATCACCGAAAATTAT | |
| (AtSUC2) | TCTAGTGCCACTTGCTCGGATCGAAATTCGAAAGTTATATTCTTTCTC | |
| | TTTACCTAATATAAAAATCACAAGAAAAATCAATCCGAATATATCTAT | |
| | CAACATAGTATATGCCCTTACATATTGTTTCTGACTTTTCTCTATCCG | |
| | AATTTCTCGCTTCATGGTTTTTTTTTAACATATTCTCATTTAATTTTC | |
| | ATTACTATTATATAACTAAAAGATGGAAATAAAATAAAGTGTCTTTGA | |
| | GAATCGAACGTCCATATCAGTAAGATAGTTTGTGTGAAGGTAAAATCT | |
| | AAAAGATTTAAGTTCCAAAAACAGAAAATAATATATTACGCTAGAAAA | |
| | GAAGAAAATAATTAAATACAAAACAGAAAAAAATAATATACGACAGAC | |
| | ACGTGTCACGAAGATACCCTACGCTATAGACACAGCTCTGTTTTCTCT | |
| | TTTCTATGCCTCAAGGCTCTCTTAACTTCACTGTCTCCTCTTCGGATA | |
| | ATCCTATCCTTCTCTTCCTATAAATACCTCTCCACTCTTCCTCTTCCT | |
| | CCACCACTACAACCACCGCAACAACCACCAAAAACCCTCTCAAAGAAA | |
| | TTTCTTTTTTTTCTTACTTTCTTGGTTTGTCAAAG | |
| Exemplary | CAGCGAAAACACCTTTGATGGGAGCGGTATCAGGAGGCTCTTGTCCAA | 60 |
| Arabidopsis | TAAATTCGAATTCGATAAGGTAAACTACCATACATATATATGTTATCT | |
| thaliana 5- | AGCTTTTATGCTAAAGGAAAACTTTTTAAATGATGGTAACGAGTGATG | |
| methylthioa | ATGATCCGGAACGGTTTGGTCGCAGGCACTAAACGTTGCCATGGAGAC | |
| denosine | GATTCCAAAAGACCGTCAGGGTAAGGTGTCTAAAGGATATCTACGAGC | |
| nucleosidase | TGTGCTTGACACTGTTGCACCATCGGCCACTTTACCACCAATAGGCGC | |
| 1 gene | TGTGTCCCAGGTAAATAATGCCCCGTCTAAATTATTTTGTCTTTTAAA | |
| promoter | TTGTTTATTTTGCCTTTGAATTTACATGTTACAATTATTTGTTAAACA | |
| (AtMTN1) | AATGAAACCAGAATTAGTGTTTTAATCAAAAATTATTAGTGAATTTTT | |
| | ATTTTTATTTTTTGAACGGCATTGATTAGTTAAGTTTGTTTTTGTTTA | |
| | TAAGATGGATAATATGATAATGGAAGCGTTGAAGATGGTGAATGGAGA | |
| | TGATGGAAATGTGGTGAAGGAAGAAGAGTTTAAGAAAACAATGGCAGA | |
| | GATATTGGGGAGTATAATGTTGCAGCTCGAGGGTAGTCCCATATCGGT | |
| | TTCCTCTAACTCGGTGGTTCACGAGCCGCTCACCTCGGCTACCTTTCT | |
| | GCCGTCAACTTCGACTGATACAGAGGAGCCTTCAAACTAATCATAGAA | |
| | GGGAATAAGCAGCACTAGCAGCAACAAATGTTATATGGTTTTGACTTT | |
| | TGAGTGTTTACCCCCAAAAGTTTTAGATTAATGAGGAAAACCGTCTTT | |
| | ACTTTCAGATGTATAAAATTGAAAGTTTGGGGTTTCCTCTTGTTGGTG | |
| | TGGTGATTCTACTCATGCCTTTTTTTTTTTTTTCTAATGACCATGGGA | |
| | TGCAATGTTTACTCTGTTTTTTAATTTCGTTAAAATTTGTTTACGTTT | |
| | ATGATGCTTGAATGGCTATGATGAAACATTTGAGTTATCTTTAAAAGT | |
| | GTGAAATAAATATTCTGAAGTTAATTGAAGAATTTGAAAATTTGATTA | |
| | CAAGAGCTTGGCTAAAACTACAAGGAGACCAGATTAGTACAAAAACTT | |
| | AGCTAAATTTAATTAATTACGGTCATTAGCACAAAAAAATAATTTGTT | |
| | TTTATTATATTATTATTGGTAAGTGGAAACACAAAAGAGGACCAAAAG | |
| | GTCCAAAAACGAATAAACTGTATCTCTCATTCGCCGGAGTTTCCAGCC | |
| | GTTTCTTTCCGATTCTCGGATTTTTCCTGGGAATCAAACGCATCGCCG | |
| | AGAATCGGAAGAGAGGGATAAGGTT | |
| Exemplary | TCTAGATGACTTGGATTAATTCTCTAACAAGAATTTAGTTTAATTGAC | 61 |
| Cucumismelo | ATTTGTATGTTTGAGGACTAAGAGGACTTTAGTTTTAATTTCTAATCT | |
| galactinol | AATTTGTACTAGAAAAGAAAAAAAAAGAGTCGGATTAATTCTCTACCA | |
| synthase | TTGAGTGGAGGATACTTGGATGCAGTTCAAGTTCTCATCTCTCCAATT | |
| gene | TGTCACGTGACAGCGGATGATTAAGCATATGAGTAGGCTGCAAAAGAT | |
| promoter | TATAGACGTAGAAGATGATACCCAATACAAAGGCGTAACTTTTCCCGG | |
| (CmGAS1) | ATGACTTTTATACTCTTTACAAAATTGGAAGTCCTATTCTATCTACAT | |
| | CTTAATTTCCAGTTGTTATAATGAAGAATAGTCTGAAAATGATATCAA | |
| | TTTTTTCTTTCTCAATACCATTCAATTACGTTAAGATTATTAGGAGCT | |
| | GCCATTATTATTATTATTATTGTTGTTGTTATTATTATTATTATGCAA | |
| | CCAAGTTTGATTTGAAATTGTTTGCCAAATTTTACTCCAATTTGATGT | |
| | TGTTTAATTACTTTAGATGGTATAATAAGAATGAAGTTGAATTTAAAG | |
| | AAAAGAAACAAAGCTTGAAAGAATGGAATACTTAGGTGTAGAAGAAGA | |
| | CAACGTATTTATAACGTCGTATAGTGTAAATAAAAATGCACACATTTG | |
| | GATGCCCTTTATGCTTCTTAGAGGTCAGACTTTCCCACAAAGGCTAAG | |
| | GTGATTCAATCGTGTGGGACATCTTGTTCTCCCATTTGATTCTCGTTT | |
| | TCATTAGACCAAAATTAACAAAAAAATAGTAATAATTCTATTCTTTTT | |
| | AAAGTTTGTGATATTACGGTTTATCCTTTGTTAAAAAAGTTTATCTTT | |
| | GAATGTAAGAATTTGATAGAATGTTGAATGAAAATTAAGATTTTGAAA | |
| | AGTTTTGCTGAATTTCAAATAATATAACTCTCTAACTTTGGTTTAGGA | |
| | AAATTAAGTGATGACAATTATCTCTATTAGAATTAGTATTATAAGTGA | |
| | TATTTGAGTTATGCACTTGACTTGGTCGTGTTGGTAAATTCTTTGGAT | |
| | ACAGAACAAAAGAAGTTGCATGCCAAGAAAGATTTCTAATAGATATGG | |
| | TGAGATATGTGGCCGTTGGCTCTATTGGATTGGTGGTATGTTCCAGAG | |
| | AAGAGGAGTGCGTATGGATACGACCTAGGTGGATAAATGATTATATGA | |
| | GGAGATGGTAATTTTATGAAATGTGTTAGAGCTTTGATGTTAATATAT | |
| | ATTTTTTAAGTGTGTTTTGTGATCGATGGTATTAGATGAGTTCCTTAT | |
| | TAAACATGTTTTCTTGGTTTTTCTCGAGGTGGGGTTCTCAACACTTGG | |
| | TAACATGCATCATGTCCACGAGATGTTCTTCATCTTATCTCTTGTAAT | |
| | ATTATATATGATATCTCACACAATACAGGTTCGTCTGAAAAATCTTTC | |
| | TTTATTTGAAATTTTTTAGGTATTTATTCTTGAGGATTTTTTTATTCT | |
| | TAAGTAAAGTGTTCATGATTTGAAGTTAGAAATATAGGAGTTATTTTT | |
| | AAGAGAGAGTCTCACACTCAAAGGGAGTCTAAATATCTTTTTTACTAA | |
| | TTTAGGTTGTGTAATAACCTTGTATTTATCGATAAGTATCACGATGTA | |
| | ATCATTTAACTATCTATTAACGAAAATCTTTTTTAGGACACGTTGCCT | |
| | CCTAGATAGATGCAAGTTGTATTGCAAAACTTGTACTCTGTTTTTTAG | |
| | TTTTTTACATGTTTTACTTTAGAACTAAACCTAAGTTATGTTATGTGT | |
| | CAAATAAACTTCTTTAAAATAATATTAAAACTTCTCAAAATAATAGGA | |
| | AAAAAAAGAAAAATTTCAAATTTAATATATATATATATATATTGTAAT | |
| | ATTAGCTTTCATTATCATTGAATTAAAAATTGCATATACAAGAATCGA | |
| | ATAATGTGGAGAAAGTAGTTTTCCTTTTTCAACTTTGTGTAGAGGCTA | |
| | AGTCTCTAAAATATTGGCTTCGACTTTGTACTTTTGGATCCGCCACCA | |
| | CAATCAGACAAACTTCCATTTGATCATTACCTTTATCGAATCAAATTC | |
| | TTTCCCTTCCAATCTGTCACAATTTTGAACATACCATCCACCTTCTGA | |
| | TTTTTTGATTCTAAATAAACCTTATTAGCAGAGATTTTTAAAATTAGT | |
| | ATTAAATTATACCAAATACCCTAATGAACTTTTTCAATAGTTTTTCTA | |
| | TTTTATTTTTTTTTTCTTTTGTGTGTATGAGTTTTTTCACCACCATTA | |
| | GAAAACACATTTGAAATATACAGAACCAAATTGTTTAATTTGAATTGG | |
| | TTTTCCATACCATTTTTACAAAATACATAGTATAACCAAAAGAACTAT | |
| | AGTTTTAAGTAGTGTATAATAGTTTAATTTTAAAGACAAAGAACTAAA | |
| | CAATAATCATTATCAAAAACACTACCTTAAAACAGAATTGAAATCAAA | |
| | TCCATTTGTTTAGGAATATATATATATATATATATATATATAATATAG | |
| | TATCATAATATATAAAAAAAATGTCAAAATCTGAGATTCTTTGATCCT | |
| | CCCTAAATTGTCCATTTTTGTCTTGCCTACAAACTTGCAAAAAAGAAA | |
| | AAAAAAAAGGTTCATAGATAGAAATGACCCATAATTGAATCATAAAGC | |
| | AATAAGGATATACAAAATTATTATATCCAAGAGGGATGAGAGATAATC | |
| | TTAAAGGTGCAAAAGAATCTTCTTATTGATGGAAGAAGAGAATACAAA | |
| | CTCTTCCAACTTTTGATCAAAATGCCCATAATGCCCTCCATCTCACCT | |
| | TAAAGATAGGATATTCCAAGTCATATTCATCCCACCAATACCAATATC | |
| | TAAAATAATAAGTAACAAATAATTACAATTACAAATATAAAGTGCATA | |
| | GAAATTAAACTTAGGGGTATCTATAAACTTAAAACAATGTTCCCCAAG | |
| | GCTCTATAAATAGCCTCCTTCCCATCCCTTCACAACTCAAGCTTGAAG | |
| | GACTAAAACAAGAACTTGTAAGCTTGCCCTTCTTATTAAGTCCTTCTT | |
| | GCCTCCCTTCCTTCGGAGAGAAAAAACTTTTGTTGTTTCAAAAGCACC | |
| | AAAGTCAATATGTCTCCTGCA | |
| Exemplary | AGCTACGCTCTTTGTCCACAATGTGACAAGGAATGAGAACGAGTCAGC | 62 |
| Epipremnum | AGTAGATCATCTGGCGCGCTCTCTGATTGGTGCGTTCACCTCCCGTAC | |
| aureum | CCATGGGCACGCACCCGAGCAGGACCGGGCACCCCCAGTGAGCCCCTC | |
| metallothionein | ACATCCATTTCCTGCCCTGTCGTGGAGTGCAGTCTCTTCGACGTCCCC | |
| promoter | GCCTTATAATTAATTACCTGTGCGTATTCGTCCGCACGCTACTGTGCA | |
| (rrEaLeaf1) | ACGATTCCACCATAGGATATATGAGGGGCTTATGCTTATCATATGGAG | |
| | TTCAAATTTTCTTTTTTATTTTTTTTTATTTTTTAATTTTTTTATTCA | |
| | TAGTTCTAGTTGGATTTTTGATATTAGAGCAGGTCTTTTTACAAAGAT | |
| | GCTATTTTTGTGAATTAAATTTACGAATTTGTCATCTTTATTTTAATA | |
| | TAATCATAAAAATATGTATGATAATATAACATAAATTCATGTGCAACA | |
| | ATGACATATTTGTCAAAAAAAAATTATTAAAATAATGATTATGGAAGA | |
| | GGAGAAGATATAGAATTAAAAAATCAGATAGGACAAGAGAAGAAGATA | |
| | AATCAGAACTGGCCATCCTTTGAATTCAAGTTTGTTTTTAGTTTATTT | |
| | AATTTTTAATTAATTTTATGTGGTCCGACCACAGAAAAAGAACAACCC | |
| | TAAATTTAGCCTTCAATACATTACTGTGGTGCGAGGAAGCTGCGTCCC | |
| | CATATGCCCATGGCGTGTGGAGCTGGTACGACTGCTTCTGTCTCGACG | |
| | TGCGTTCCCCCCGGAAGAAAAAGAGAAGGAAGTGACGTGAGAGGTCCA | |
| | GAGGCAGCCGACCTTCTCCTCCATTATCGGGAGAGATTCCTCTCGGGA | |
| | CTCCCACTCGCAAGAGCCCTCTC | |
| Exemplary | TTGTTCAGAAAGGAACCCCCTAGTTTGTAATTGGAGGTCATAAGAGGT | 63 |
| Epipremnum | ACTTTCAGTCCTCAAAATTTATCATTTCTTAATGAAATTTTTAATTTT | |
| aureum | AAAAGATTTATTCTTTTTAATAATTTTTAGGTTGAGATCAAGTAAATT | |
| ribulose | TAGAAGATGATTTTGACAACGATTTTTTTGAAGTAGATAATCAAAATT | |
| bisphosphate | AGGAGTTTTAAGAATGATAATAATTATTATTTTAATAAAAATTTAAAC | |
| carboxylase/ | TCACCTTCTATAAACAGATGTCTCTCATTGTACCAAAAATTTTAGATT | |
| oxygenase | TACATATTATTATAAAAATATCTTTTCATTTTATAATTTATAAAAATA | |
| activase 2 | TTTTTTAAAATTAATTTATTTCAAAATCTATCATGAGCTGTCTTAAGA | |
| promoter | TAAGAGTTGCATAATTATAATTATTTTTTAATTGTAATAAATAAATAT | |
| | CCATACTACCCTCATGTTAAAAAAATATATATATATATATATAAAATC | |
| | ATCCCTCCCCCTCTCTCTCTCCTCGTCTCTTATGTTTCTGAATCACAT | |
| | TTTTTTAAAAATATTAATTAAAAATAAAATATTTTTAAATGTTTTAAG | |
| | TATAATAATATCTAATTAAATTTTTTGAAAACATTTTTTAAATTATTT | |
| | TATAAATGATAAAAGAGATCTTTTTGTAGTGCCAGCTCGTAACAAGGT | |
| | ATATTTACGAATAACCCTTCCTTTTATTGCAGACACCTCGGCTGAGAG | |
| | TACGCAGTAGATGACGGGTCCCACTTTTTTTCCCCACGCTCCAAATAG | |
| | CTCCAACGTCGTCAGGACACGACTTATCTGAACAGAAGTTATCCGCCC | |
| | TGATTGCGCCACGTGTTCCGGCCCAATCCCCACTGTGTGGCCACAGGA | |
| | CCCTCCGCTCTCCCCCTCTCCTCCCCTCCCCTCCGCCAGCCAGAGGGA | |
| | AAAGGAACAGAACAGGGCGATCTCCAGAACCTCCGCAGGCCGCTTTAT | |
| | ATATAGTTCGCCCTACCCCACCGCCTCCGGCCAACGCTGCTACGAGGA | |
| | GCTGAGCTTTTGGTGGAAGCGGCGATCCCCCCCTTCCGCCTTCTAGGT | |
| | CTTCCGGGTCCC | |
| (rrEaLeaf2) | GTGCGATCCCTCTTTCCCTCCACAAATTAATAAAGCCTGATTTGGGTT | 64 |
| Exemplary | TTGATCACAGAAGATCTGTGTTGCTTGATCGATGTGTTGATAAAGACT | |
| Epipremnum | AAAAAGAAAAAGAAATCCTCGATCTATTAATTTAATTTTTAAACAATA | |
| aureum | AATTTACCTATTCTCTTTCCATTCCCTTCAGTCTTCATGGTTTCATTA | |
| hypothetical | ATGGCGTTATATGCCCTTGTGAGAGATTTAATTGCGTAACTATCTCTT | |
| polypeptide | TTAGATTTGCATCTTCACGCGCATGTCATCCTCATGCGGCAATGTACC | |
| AQUCO_03 | TATCTATCCCTCCCGTGAGGGTATATATACGATTAAAAGTATCATCAA | |
| 600155v1 | GATATTTTTAAAATTTACAGCTATACACCTCTTAATGATATAATGGCA | |
| promoter | CACACGTTTGAAGGAAGAGAGTGTATACACACGAATGTAAATTTAGAA | |
| (rrEaLeaf3) | AGGATATTCATGCAAGTGGGACTCTAATAGACATGTATGGAAAATGTC | |
| | TGTTTTTTTTTAACCCATATCCAATTCACTCGAGTATAAATGAAGGTG | |
| | ATAATTATTTGCATGTGCTTGGCCTTTTTAATGTAAATTTGGTTTATA | |
| | CCAGTGGCATGTATTCAAACTTCCTTTATTTTTCGGTCTGCATCCATC | |
| | TCCCTCTCTCTGGTGTCTTCTTCTTCACGCAGCCAGAGGTTAAGGGAG | |
| | TTGCGTGTGCAAGTGCAACTGGGCAACAGTGCAAGCATAGCCAAAGGG | |
| | AAGAAGAAAGAAGAGGAATTGACACGAGAGGTGGAGGGGTAGCCCCCC | |
| | TCCTTCCCCACCATAATTGAGATTCCTTTGGAAGCTTCCTCCATGGAG | |
| | GCGTGTGCCCATCACACACAGGGGCCCTCCCCTCCCCTCCTCTCCTTG | |
| | TGCCGTGTGCGTCCCTCTGCCATCCCCCCCTGGGGCCTATAAATATCG | |
| | TCGCAGGGTGGAAGCCCCTCCACCATAGCTGGAGCTGACCCCTGAGCT | |
| | GAGAGATATATAGCAGAAGCTCTCTTTGATCATCTCTAGAGGCTCCCC | |
| | TCTGC | |
| Exemplary | CGCACGTAGCCTTCGTTACTCATCTTGTTGTTCGTCTAATTTGGAGAG | 65 |
| Epipremnum | ATGGTTTCAAGCATTTGACAATCCAAGGAGACAAAGTCATTAGTATTA | |
| aureum | ATGTTTCTCTGTTAATTAATTGTCTCCCTGATATCCTGTCTCAAGTAT | |
| carbonic | GTTTATGTGTGTGTGTGTGTGTAAATATAAATATAAAGAACAATATGT | |
| anhydrase | GATAAAGGATAACCATTCTGCATGGTGGATTTGTCTTCATTAATTAAT | |
| 2-like | ATAGTTCTTTCTTTCCATCATTTGATTTCATTTCATACACTAGTACTT | |
| isoform X1 | TGGTACCATGTTTATTTTTCAAGGTTTATCGAACAGGAATTATTCAGA | |
| promoter | AGATATACCAAAAATCGATTGGATTCATTCTCTATTCAGACTGTTAAT | |
| (rrEaLeaf4) | TGTTAACCATCGATTTAAACATGTCATCTTAAGGGAAATTAAGAAACT | |
| | AGATTGTGTTTACGTTTTCCACACTGTTAGACCTTCTATAGTATCTTC | |
| | ATTGTTCTCGAGTCGATTGGTAGTATTGGAACGAACTAGCATGCATGT | |
| | GTGGAACACCCCCTCTTATATACTGCAAAAAATGAAAAAGAAAAGAAA | |
| | ATGGACCATCACTTTGATTTTTTAGGGTTTGGTGGCTTCAAGACACGA | |
| | TGCTTGGCTGGGTGCAATTAAACTGTGCCATAAAAATGTACTATGCTA | |
| | TTCAATAATCGATTTCATGAGACATGGTACATGTCATATTTCATAAAT | |
| | GACGTGGTACATGCCAAATTTCATAAGTTTTCTTGTCTAGAAACTTAA | |
| | TAAATTACTATTCGCATAGAAATCCTGAATTTTTACTATTTCTGATTT | |
| | CCCCCACCCCCAGAATTTTAAGGTTGAAGCTATCAGAAAAACAAGAAT | |
| | TATTATATATAATCCATCTGCAATGCATGAGATTAGCGATACACCTGC | |
| | AACGCCATCACCTATTCCATCCAACGATTACATGACACTGTCATCTCC | |
| | AAGCCTTCTCTCTCTCTCTCTCTCTCCCTCTCCCTTATTTGAAGCAGA | |
| | AGCCATGGTTGATCCGGCTTTCGCTTTCCTTATCCTAACCCACCCCCG | |
| | TCGCAGAGACTATATATCGAGCCCTCCACCCCTCCTGGGACGGGTGTG | |
| | AAAGAGAGCA | |
| Exemplary | CGCACGTAGCCTTCGTTACTCATCTTGTTGTTCGTCTAATTTGGAGAG | 169 |
| Epipremnum | ATGGTTTCAAGCATTTGACAATCCAAGGAGACAAAGTCATTAGTATTA | |
| aureum | ATGTTTCTCTGTTAATTAATTGTCTCCCTGATATCCTGTCTCAAGTAT | |
| carbonic | GTTTATGTGTGTGTGTGTGTGTAAATATAAATATAAAGAACAATATGT | |
| anhydrase | GATAAAGGATAACCATTCTGCATGGTGGATTTGTGTTCATTAATTAAT | |
| 2-like | ATAGTTCTTTCTTTCCATCATTTGATTTCATTTCATACACTAGTACTT | |
| isoform X1 | TGGTACCATGTTTATTTTTCAAGGTTTATCGAACAGGAATTATTCAGA | |
| promoter | AGATATACCAAAAATCGATTGGATTCATTCTCTATTCAGACTGTTAAT | |
| (rrEaLeaf4) | TGTTAACCATCGATTTAAACATGTCATCTTAAGGGAAATTAAGAAACT | |
| V2 | AGATTGTGTTTACGTTTTCCACACTGTTAGACCTTCTATAGTATCTTC | |
| | ATTGTTCTCGAGTCGATTGGTAGTATTGGAACGAACTAGCATGCATGT | |
| | GTGGAACACCCCCTCTTATATACTGCAAAAAATGAAAAAGAAAAGAAA | |
| | ATGGACCATCACTTTGATTTTTTAGGGTTTGGTGGCTTCAAGACACGA | |
| | TGCTTGGCTGGGTGCAATTAAACTGTGCCATAAAAATGTACTATGCTA | |
| | TTCAATAATCGATTTCATGAGACATGGTACATGTCATATTTCATAAAT | |
| | GACGTGGTACATGCCAAATTTCATAAGTTTTCTTGTCTAGAAACTTAA | |
| | TAAATTACTATTCGCATAGAAATCCTGAATTTTTACTATTTCTGATTT | |
| | CCCCCACCCCCAGAATTTTAAGGTTGAAGCTATCAGAAAAACAAGAAT | |
| | TATTATATATAATCCATCTGCAATGCATGAGATTAGCGATACACCTGC | |
| | AACGCCATCACCTATTCCATCCAACGATTACATGACACTGTCATCTCC | |
| | AAGCCTTCTCTCTCTCTCTCTCTCTCCCTCTCCCTTATTTGAAGCAGA | |
| | AGCCATGGTTGATCCGGCTTTCGCTTTCCTTATCCTAACCCACCCCCG | |
| | TCGCAGAGACTATATATCGAGCCCTCCACCCCTCCTGGGACGGGTGTG | |
| | AAAGAGAGCA | |
| Exemplary | TTCGATCTCCCCCTCGACTTGAAAAAACTAATAAAAAAATGTAACCTT | 66 |
| Epipremnum | ATATTTTTCCGTAAGTAAAACGGAAAGTATATTTAATAGAATATAAAA | |
| aureum | AATCTGTAATTTAATTATTATTCGGATAATAAGAGAAAGAAGAGGAGG | |
| beta- | GCAAAATTATGGGAGTTGATGGATGGATGATGCTGCCACGTCAGAACT | |
| galactosidase | CGGACCGGGACGTGGCCGGCCGGGTGGCGCCGGTCCTGCCCGCCCACT | |
| promoter | CGCTTTCACCCCACGCCCTTTAAATCCCACCCGGCGCCCCGTTTCCCT | |
| (rrEaPetiole1) | CGCCACGGCCATCACCACCAACGGCCTCTCTCTCTCTCTCTCTCTCTC | |
| | TCGCGATCTTCACAGCCACTTCTCACTCCATTACGCTCTTGTTTACTC | |
| | CTCACTCCCATCTCCTTAAACGCAAGCGACTGCAACCCAAACCACGCT | |
| | CTTCCATTGGCCTCGTCCTCCTCTCTCGTATCCCGAAAGCGAGAGAGG | |
| | ACCGGCCAGAGAAAGGGGACAGAAGAAAAAAAAAAGAGTCGGAGGGAG | |
| | AAAAAGAGTGGGCCGAGCGAGAGGAGTTGGAGAGAAAATTATACTGAA | |
| | GAGCACCCTAAAGCGGGCAAGGAATATTGCTGGGGAGTTGGGAGGAGA | |
| | GAACAAAACGAGAGAAGGAAGAAAGAAAGGAAGAGGGAGACGCGCAGT | |
| | GTTACAAGGAAGATTAGGGGATAAAAAAAGCCGTTTTCTTCTTCTCTG | |
| | CTGCTGCGAGGTCGCTGACCGCCTTCCTTAGACTCCTCTGCTGGACGC | |
| | ACTACTTCCCATCTTATCTTAGCTTTCTCCAACCTTTAGCTTCTGACA | |
| | CATTAAAGAGGAGGGAATATAGAGGAGAAAAAAAAAAGATCGTCGGAA | |
| | GGAAGAAAGGAAAAAAAAAGATCCAACCAGGTTTCTGCGGAAG | |
| Exemplary | TGGTTGAAGTGCTAAATTTGGCATTGCCTCAATTTTGTTACTAAGATT | 67 |
| Epipremnum | TTTGTAATATCAAAAATTAATATTATAATTAATTTAACACAAAGTTGA | |
| aureum | AATAATTCAGATGATCTTGTCAAATTATTAATACTGTTGATGATATTA | |
| vacuolar- | CACTATTTAATAAAAGAACCATATGCCCCATAAAATTAACTCGGCCTT | |
| processing | CACTGAAGAATGATCAAGTGGTCATTATGTAATCATCTGAAACTCAGG | |
| enzyme | GATGATACATACACATACATGTCTAAAACTCCTAGAAACTGTAGTTAA | |
| promoter | TTGCACCCTTTTGCCACTGCATTATTTCATCTGGTACCAACTGACATG | |
| (rrEaPetiole2) | GCATCCCCTGTCCACTTGCTATTGGATCAACACGCCCGACTTCTTACG | |
| | TCGCCACGCCGGGGCCCACCTAGATAGGAACTATCTGCTTGATCCCGT | |
| | CGAATCAGCAGCGTTCCAAGCCCGCTCCCCCATCGGATAGATATTAAC | |
| | CGTCGGATCAATGGATCCATCGTGGGAACATCTATCTTCCAATGCCGA | |
| | ACAGCACAACTAACTCCCAACCGCCACCGCTGGCCCACCCACCGATCG | |
| | TTGAGCCGGATCAGGATCCTGCGGCCCTCACGTGACCCCCAGAGAACA | |
| | TCGCCTCCTCATAGGCCGTCGCGTGCGAGGGCTGACGCCCGTCAACAC | |
| | GACCCCCAGGGAAGACGTCACGTCGGCAATTCCGGAGATTCAAGGCGA | |
| | GCGCATAGGCCGCGCCAATTAAGCTAAAACCCGAAGAAATCCTTCGAG | |
| | CAGAGCAACAGCTCGGCGGGGCCCCACTTTTTCTAACTTTCCCCCGCT | |
| | CCAGTCTATAAATAGCGCCCACTTTCCGCCCAGGTTTCCTCGCCATTG | |
| | ACGATTAGAGCACTCGACGGAGGTAAAGCTGCTTCCCTGGGTGCCCCC | |
| | CGCACCACCACCAACG | |
| Exemplary | CTGAGGAACCCCATTGCAGTTTTACTACGGTCAGATTGGAGGAGAGAT | 68 |
| Epipremnum | CGAGGCGGCACACGTAACGGCAAAACGTCACGTTGACGGGGCTCTTAT | |
| aureum | GGTTCCCGTGTTACGTAAACCCCCGGCATTGGGACCATTGGGACTCAC | |
| cathepsin B | CAAGTCCCGTGTGCGATTGTCTCTCGAGTGGCGTGCCTCATCACTCAA | |
| promoter | CACAAGGGCGAGGGGTGCACGGCGCTGTCGTCACCCCTTACGTGAGCA | |
| (rrEaPetiole3) | CGCGGTATAACGATAACGGCATCTACCATCCGACGGGAAGGAACAGCG | |
| | TCAGATCGTAGCGGGATGGACCGTCACGGCCTCCTATATATCTGATGA | |
| | AGCGCCGTCAGATCGGGAGCCCTGGGCCCACAGCATTGGGGTGCAAAC | |
| | CAATCAAATGCCACTTCCTCCAATAATGGACACTATGGGTTCCAGCTT | |
| | CGAAGAAGCGGCAGCTGGCGCCTCCGTAGCTCTCTCTCTCTCTCTCTC | |
| | AAACGGCGGCGTCATCTTATCCTATCGCCTTTTCAGAGCCCGGCTGCG | |
| | CAAGTAACCGTCCCGTTGATTTAGATCTGGATTTCATTTATTTGCTAC | |
| | GTTGAAATCAGGGTCCAATCGCACTGCCATCACCCCCAAACGTCCGGA | |
| | TTCCATTTATGTTATACGCTGAATCGAGGTTCAGCCGCGTTGCCATCA | |
| | CCGTCGAAATAGGTACCGCCGCCGCCAAGCTTCCATATCATCTTCCCC | |
| | CTCATATCAAATTCTGACCCCTCTCTCTCTCGCCCCCCTTCCTTCCTG | |
| | GTCTTGCTACTCCGCTCCGTCCCTCTCCCCGTTTCACCTCTCCACCTG | |
| | CTGTCTGTAAATGGTGGGGGTGCTGTTTCGAGCTGAAGGGTGAGGGTG | |
| | TGGGGGTGCTGTTTGGAGCGGAACGGAGAGGATAGGGCACAGATATAG | |
| | CTAGGGGGAGAGAGAGAGAGAGAACAACGGGG | |
| Exemplary | GTACGCAGGCTGAAAGAAGCCTCTTTATTCAATTGAGAAGTGATAGTA | 69 |
| Epipremnum | ACTATTATCCAATAGAGTAGGGAGAAGACGTATACATCCTTTTCTATG | |
| aureum | GCATCGTTTACTTTGTCTGTCCACCATGAATGTACTCTATAATAAGTA | |
| metallothion | GTAATCAATGAAATGATACCTTAAAAAATTAGATGTTTGTAATGGCCC | |
| ein-like | CCCCTTAGTAATCTTCCTAGTGACGGATGCACTTTAAAATATTGGAGA | |
| protein type | AAAAAATGATGGTTGCAGTACAACAATATCATATTAGGTAAGAAAAAT | |
| 2 promoter | ACAAGAGTGTGTGGAGACTTGGTCTACTTTTGATGTAAAAAAACTGTA | |
| (rrEaPetiole4) | AATATTGATGGGTTGAGTTAGTATTATAAAAAAAGAATAAGTTTGAGT | |
| | AATTCCTTTTCACATAGAAACCTTTTAAGTCCCTTTCATATATCAAGC | |
| | AGCAGACAAGAATTTAAAATTTTGAGGTCTTCACATGTTGGATGCAGT | |
| | GCTCTTCTAATTAGCTGTGGCGGCAGGAGTTCATGAAAATTAAGAAAA | |
| | AAATGATATGAAAAATGACAAGATTCCCTACTTCATCCGACAATGCAT | |
| | ATGGTCTGGGGCAAATTAGAATACCACACTTCTCTCGTCATTCTGTCA | |
| | TTACTCCTTTTTTTATTTTAAAAAACTCACCTCATCATTTATAGTACC | |
| | GCATGTTAACTCAGGTGTTATTTGATAACGTTATCAGCGTTGATTTTA | |
| | TCTTTTAATTTTTATAAAATTTTAAAAAATATATAAATATTACTATCA | |
| | AATGAATAAATACTAAATCAGATTTAAAAAATAATTTATAATTATTAG | |
| | ATTAAAAATCACTTTAATTCATTTTAATAAAATCTAAGACAATCATAA | |
| | TATTGATATGATTTAAAATTTAATAAGAATAACATAACGATAATATTA | |
| | TCAAATGAAGTGTTTCAAAGATCACAAGTTATCCCATGTTCGCAAGAA | |
| | GGGTAATATAACTGTTGACGGCACAACTATTGTAGGAGTTTTAAATAA | |
| | AGATCTATATAACTTGACATGACGTGAGGTAGCAGAGACCATCAAGA | |
| Exemplary | CCCGATGAGCACCTCAGATGTCCATTTGATGCTCTTTCGTGAAGTGGA | 70 |
| Epipremnum | TTCTCTTTGACGTACACATCTTATAAATATCTATATTCGTCCACACCG | |
| aureum | CTGTGCAACGATTCCCTATGTGATATATGCTGCACGGACGGAGAGGGC | |
| metallothionein | GGTTGCCTGAAGGAACACATATGCTTATGTGGAGCCCAGTTCTCTTTA | |
| promoter | TACTTTTAGTTGGCTTTGATTTAGTTTTTTTTTTTTTTTTTTGAAGTA | |
| (rrEaStem1) | GGAGCAGATCCTGTGTTGTTGCAGATTTACTACCTCGGCTGCCACCCA | |
| | TAGAACAAGATCATATTAATCTGTCTCTTGGAGCTGAAATATGGGGAG | |
| | CAAAGAAAGGGTATTAGAAAGATTCTTAAAATTAGTAGACCTGTCCTA | |
| | AGACACTGGTGATTGAGCAGTGGCATCTGCACTTGTGGACTGTGTGCT | |
| | TGTGCATGGACGCTGGCTGGAGAGATCCGCCGACGTGCATGGCGAGGG | |
| | TGCATCAATAGGACTGGACAAGGGAAGAAGAAACATCTGAACTGAGTA | |
| | TCATGTGAAATTAAAACTTTTTAATAATTTTATTTTATTTTAAATTAA | |
| | TTTTATGTGGTCCGACCACAAAAAAAACTTACAGAACATTACTGTGGT | |
| | GTGAAGAAGCTCCGTCGCCATGCTACTGGCGTGTGGGGTCGGTAAGAT | |
| | TGTCTCTGCCTCGACATGTGTTCCCCCCTACAGAAGAAAAAGAGAAGA | |
| | AGTGACTTGAGTGGTCGAGACGCAGCCACCCGTCTCCTCCATTATCGA | |
| | GAGGGATTCCTCTGGGGAATCCCACTCGCAAGAGCCCCAGCAATGCCT | |
| | ATAAATACCGGTGGAGGCGGCCCCTCTCCAGCTCACACAGAGCCGACG | |
| | TGATAAGCTCCTCCTCTCGCTTCAGCAGTTCTCTCTTGCCTTCGCCAC | |
| | TTCCCATTATCGCC | |
| Exemplary | TGTGAGTGACCAAGTGTGCTTAAGAGCAACCAAAGACTTTGGTGAGCA | 71 |
| Epipremnum | TCATAGTGCATTATGTTACCCATCAAATATCATATTGCTCATCAAAAG | |
| aureum | TTACTCTGTGGATAGCACAACCTACCATGTTACTCATATAGAGGTGTC | |
| dormancy- | TAGTGAATAACAGGATGTTTTGATGGATAACATAATACATCATACTAC | |
| associated | TTACTAATACATTTAGTTGTTCACAAAGTATCACATTATTTATTCATC | |
| protein 1 | AACACATTAAGTTACTTATGGGCATATAAAATTACTTAAAGTATCCCA | |
| promoter | ATTACTGAGGAAAGATTTAGATGTATAATATTTTTAACTTATTTCTAG | |
| (rrEaStem2) | TACAAATGGGGTGCACAAATAGTGAACAGAGTGAGGTCATTTTCTGAC | |
| | AATTCCATTGGGTAATTTTTTTTTACTCTCTTTTTTCTTTCAAACTGA | |
| | TTCAAAGAGTTTAATGGTGACAGAGTCACATATCTAGAAGAATATTAT | |
| | TGGGGGCGGGTGCAATGTTGTTTGCACTACAAGTCGACGACCGGTCGT | |
| | CACGTGGATCCCATAGTGGGCCAGGTCCATGCTATGATAAAGCCCATC | |
| | AAAGGGCAGATATTTCCGTCGTCACGTGATGGAGGGGGGGCCCAAATC | |
| | GTCTTCATGCTTATCCGCTACCTGTCCATACCGCCATCACGTCACTCT | |
| | CCCACAGCTTTGATCACTTCCGCCCCCTCCCGCCCAGCTACCCTCGAG | |
| | ACCCGGTATTCGGACGTCTTCTCGGATCCGAAATATCCGCTGTTATCT | |
| | CGGGTTTTCTTGTTGGAGTCTCATCCTCCCCTTCACTTGAGACGATCC | |
| | GGACTCGATCAGAGTGTTAAAGGATGGGGATGGAGACGTGTGAGTGAG | |
| | GGCAAAAGGAAACCTACGTACAGGTTGTCTGAAGGAAACTTTTTCCAG | |
| | CACTATCCTGCTCTCGTTACCTGTGACTATCCGTTAATTTGGCATCTG | |
| | AGCAGAATCTCTTTCTATATATGGAGTTGGCGAGGGCAGCAGCAATAG | |
| | GGGTGCAGAGCCAGTGTAGTTGTGGTTGAGAAGGAAG | |
| Exemplary | CTGAGGACGCTTCGAGATCCACTGACCATGCCACTTTTTTTTTACGTG | 72 |
| Epipremnum | AACGAGGCAAGTCGGCATTGACGAGCGGGGATGAAAAGGGCCGTGGAG | |
| aureum | CGAAGGGGACACGCACGCTCATAATACTGTTCTGTACGGCTTATATAG | |
| dehydrin | TATAAACAGATCCAGCGCAGCGCCCGCGCATGTGGCGGGGTATTGGGG | |
| COR410- | GAGGCGATGGCGCGCGTCTGCTCCCCCGCCGTGAGGCCAAGGACCTCC | |
| like | GGTAGGGGCGCACCGCTCGCGGTGTATGGCGGCCGTACCGTGGACATG | |
| promoter | CATGTATGGTGGGCTTTTTTTAAGTTTGCCCCGGATAAGTGTTACTGT | |
| (rrEaStem3) | TGTGGACATGCACATGCATACGATGATGGGGTCCGTCTGGGTCCGTTG | |
| | CTCTACTCATCCGATGCCACGCAAGCTCTGTAGTAAATGTATGTATAT | |
| | ATTCGTGTGAGAAAGAGGAACGAAAAGGGACAACTAAGCGAAGTCCGA | |
| | TGGCTCATCTTAATGATTAAATTACAAAAAAAAATTATTTAGATATCT | |
| | TCGTATCAAGTCTCTAGAGAATAATCTGTCATTTAAAGTTTGAGGTTA | |
| | TTTTATGGATATTTCTTTCTCCTTTAATGACTTATAAATATTAGATTT | |
| | TACTTCTCTCAGTTATAAAATCACTCATCATTCCAACTGAGTTATTTA | |
| | TCTAAGATTTGATGACAAGGGGAAGACGATTACGATGGGCGCTCTCCA | |
| | AGCGTTGCTGTGGAATTTCTCGCGGTGAGTGGCGATGACACGTGAAAC | |
| | TTTGTCACAACTACTCCAAGAATCCCACTAGCCATTAGCTTGTATGAT | |
| | ATTAATACTGAGACTGGTTATTAACAAACATCTAACACCACCTTTTAT | |
| | TTACCAGACGAGGACGGTAACGGAAAACAGGGGAATGAAAGCAAGAGA | |
| | AAGCCGACATCGGACCGACGTTCCTCGAGGCCCGATCTGATCCACTCC | |
| | AACCCGCCATCGTCAGCATCACCGTCTCAAATCAAGTCCATTTATCGC | |
| | CCGCTGCGAAAGGGAAAGGCAAAGGGTTTGAAAAAAAAAAAGAAAGGC | |
| | AACGAAAGGGGGACGAAGGTGG | |
| Exemplary | CTGAGGACGCTTCGAGATCCACTGACCATGCCACTTTTTTTTTACGTG | 170 |
| Epipremnum | AACGAGGCAAGTCGGCATTGACGAGCGGGGATGAAAAGGGCCGTGGAG | |
| aureum | CGAAGGGGACACGCACGCTCATAATACTGTTCTGTACGGCTTATATAG | |
| dehydrin | TATAAACAGATCCAGCGCAGCGCCCGCGCATGTGGCGGGGTATTGGGG | |
| COR410- | GAGGCGATGGCGCGCGTCTGCTCCCCCGCCGTGAGGCCAAGGACCTCC | |
| like | GGTAGGGGCGCACCGCTCGCGGTGTATGGCGGCCGTACCGTGGACATG | |
| promoter | CATGTATGGTGGGCTTTTTTTAAGTTTGCCCCGGATAAGTGTTACTGT | |
| (rrEaStem3) | TGTGGACATGCACATGCATACGATGATGGGGTCCGTCTGGGTCCGTTG | |
| (2) | CTCTACTCATCCGATGCCACGCAAGCTCTGTAGTAAATGTATGTATAT | |
| | ATTCGTGTGAGAAAGAGGAACGAAAAGGGACAACTAAGCGAAGTCCGA | |
| | TGGCTCATCTTAATGATTAAATTACAAAAAAAAATTATTTAGATATCT | |
| | TCGTATCAAGTCTCTAGAGAATAATCTGTCATTTAAAGTTTGAGGTTA | |
| | TTTTATGGATATTTCTTTCTCCTTTAATGACTTATAAATATTAGATTT | |
| | TACTTCTCTCAGTTATAAAATCACTCATCATTCCAACTGAGTTATTTA | |
| | TCTAAGATTTGATGACAAGGGGAACGACGATTACGATGGGCGCTCTCC | |
| | AAGCGTTGCTGTGGAATTTCTCGCGGTGAGTGGCGATGACACGTGAAA | |
| | CTTTGTCACAACTACTCCAAGAATCCCACTAGCCATTAGCTTGTATGA | |
| | TATTAATACTGAGACTGGTTATTAACAAACATCTAACACCACCTTTTA | |
| | TTTACCAGACGAGGACGGTAACGGAAAACAGGGGAATGAAAGCAAGAG | |
| | AAAGCCGACATCGGACCGACGTTCCTCGAGGCCCGATCTGATCCACTC | |
| | CAACCCGCCATCGTCAGCATCACCGTCTCAAATCAAGTCCATTTATCG | |
| | CCCGCTGCGAAAGGGAAAGGCAAAGGGTTTGAAAAAAAAAAAGAAAGG | |
| | CAACGAAAGGGGGACGAAGGTGG | |
| Exemplary | ACATGACACTAGGCAGGATCATTCAATACAACTAACTTGAAAGATAAT | 73 |
| Epipremnum | GAAAGAAAATAACAATAAGTGATTACAGTGTTAGCATTAATTATTTTT | |
| aureum | TATTATCTTCATCTTTTGTCCCACTAGTATTAAATACTTAAAAAATGT | |
| ubiquitin- | TTAAATTATATGCGATCACTAAGATGAGGGGGAGAGGGGGGTATGAGT | |
| conjugating | AACTAAAAACATCTTTATATTATAAAAAGTAGTGCAATAAATATCACT | |
| enzyme E2 | CTATTTATATGTAAGGGCAAATGTACAAATAAGAGAGATTCTAGGGGC | |
| 8 promoter | TGCCTCCACAAAAGTCCCTTAAACTTGAAGATCCCTTCTAAGTTTTAA | |
| (rrEaStem4) | GATTTAACATTCTTTTTGTTGAACTAACGCAATTCCACTGAGGTTTAA | |
| | TTCAGATTTTACTTAACTAAATTAAATATTTAAAAAATATTATATTTT | |
| | AAATTTATAAAAATATATAAATTATTTTAAATATTATATTATTTTTTA | |
| | AATTATTTATAATAATTTAGATAATCCTCAACAAACCATGGTTAGAAG | |
| | TTCGAAGTTCAAACCTGTGCCCTACCGTTACCACCGTGTGGTTGCCTG | |
| | CGACCTGTTCGAACCGGATTCCTCTTTATATATCCTTTAAATATATTA | |
| | GCGCCGCTCCTCTCTCTCTCTCTGTCTCTCTCGCCGACGGCAGCCTCT | |
| | GTCCCCTTCTACGGGTCCTCGAGGAGGGGCGGGGGGGGCGGAGGGGGT | |
| | CGGTCGCACGCAGCAGGCAGAAGAGAGAAGCATTCCACCGCGCTCTCT | |
| | TCCGCGTCCGTTCCCTCCCTCTCCGCCTCCGTTTGTTCCCTGCTTTCC | |
| | TCTCAACCCTGACGGTTTCCTCTCTTCTTTCCCCTCTCTATCTAGGGT | |
| | TTCGGAGAGATTGGCACGTACCGACCGGGGTTTCC | |
| Exemplary | TTGATGGGCTGCCTGTATCGAGTGGTGATTTTGTGCCGAGCTGCCGGT | 74 |
| Promoter | CGGGGAGCTGTTGGCTGGCTGGTGGCAGGATATATTGTGGTGTAAACA | |
| PVUBI2 | AATTGACGCTTAGACAACTTAATAACACATTGCGGACGTTTTTAATGT | |
| | ACTGGGGTTGAACACTCTGTGGGTCTCATGCCGAATTCGGATCCGGAG | |
| | GAAGCCAACTAAACAAGACCATAACCATGGTGACATTTGACATAGTTG | |
| | TTTACTACTTGCTTGAGCCCCACCCTTGCTTATCGGTTGAACATTACA | |
| | AGATACACTGCGGGTGGCCTAAGGCACACCGTCCGAAACCGGCAAACC | |
| | AAGCCTGATCGCCGAAATCCAAAATCACTACCGGCAATCTCTAAAGTT | |
| | TATTTCATCCTTATATGACGAGGAAAGAAAAGAAGAGAGAAATAATAT | |
| | CTTAACTTCTAAATCAGTCGCGTCAACTTTCTCGGCTAAGAAAGTGAG | |
| | CACTATCATTTCGCAGACCATGTCATGAGTGCCGACTTGCCATATCTT | |
| | ATTATATTCTTATTTATTTAATTATAATCCCATTGCAATACGTCTATT | |
| | CTATCATGGCCTGCCACTAACGCTCCGTCTAACGTCGTTAAGCCATTG | |
| | TCATAAGCGGCTGCTCAAAACTCTTCCCGGTGGAGGCGAGGCGTTAAC | |
| | GGCGTCTACAAATCTAACGGCCACCAACCATCCAGCCGCCTCTCGAAA | |
| | GCTCCGCTCCGATCGCGGAAATTGCGTGGCGGAGACGAGCGGGCTCCT | |
| | CTCACACGGCCCGGAACCGTCACGGCACGGGTGGGGGATTCCTTCCCC | |
| | AACCCTCCCCACCTCTCCTCCCCCCGTCGCAGCCCATAAATACAGGGC | |
| | CCTCCGCGCCTCTTCCCACAATCTCACATCGTCTCATCGTTCGGAGCG | |
| | CACAACCCCCGGGTTCCAAATCCAAATTGCTCTTCTCGCGACCCTCGG | |
| | CGATCCTTCCCCCGCTTCAAGGTACGGCGATCGTCTCCCCCGTCCTCT | |
| | TGCCCCATCTCCTCGCTCGGCGTGGTTTGGTGGTTCTGCTTGGTCTGT | |
| | GGCTAGGAACTAGGCTGAGGCGTTGACGAAATCATGCTAGATCCGCGT | |
| | GTTTCCTGATCGTGGGTGGCTGGGAGGTGGGGTTTTCGTGTAGATCTG | |
| | ATCGGTTCCGCTGTTTATCCTGTCATGCTCATGTGATTTGTGGGGATT | |
| | TTAGGTCGTTTGTCCGGGAATCGTGGGGTTGCTTCTAGGCTGTTCGTA | |
| | GATGAGATCGTTCTCACGATCTGCTGGGTCGCTGCCTAGGTTCAGCTA | |
| | GGTCTGCCCTGTTTTTGGGTTCGTTTTCGGGATCTGTACGTGCATCTA | |
| | TTATCTGGTTCGATGGTGCTAGCTAGGAACAAACAACTGATTCGTCCG | |
| | ATCGATTGTTTTGTTGCCATGTGCAAGGTTAGGTCGTTATCTGATTGC | |
| | TGTAGATCAGAGTAGAATAAGATCATCACAAGCTAGCTCTTGGGCTTA | |
| | TTATGAATCTGCGTTTGTTGCATGATTAAGATGATTATGCTTTTTCTT | |
| | ATGCTGCCGTTTGTATATGATGCGGTAGCTTTTAACTGAATAGCACAC | |
| | CTTTCCTGTTTAGTTAGATTAGATTAGATTGCATGATAGATGAGGATA | |
| | TATGCTGCTACATCAGTTTGATGATTCTCTGGTACCTCATAATCAACT | |
| | AGCTCATGTGCTTAAATTGAAACTGCATGTGCCACATGATTAAGATGC | |
| | TAAGATTGGTGAAGATATATACGCTGCTGTTCCTATAGGATCCTGTAG | |
| | CTTTTACCTGGTCAACATGCATCGTCCTGTTATGGATAGATATGCATG | |
| | ATAGATGAAGATATGTACTGCTACAATTTGATGATTCTTTTGTGCACC | |
| | TGATGATCATGCATGCTCTTTGCCCTTACTTTGATATACTTGGATGAT | |
| | GGCATGCTTAGTACTAATGATGTGATGAACACACATGACCTGTTGGTA | |
| | TGAATATGATGTTGCTGTTTGCTTGTGATGAGTTCTGTTTGTTTACTG | |
| | CTAGGCACTTACCCTGTTGTCTGGT | |
| Exemplary | GAAGCCAACTAAACAAGACCATAACCATGGTGACATTTGACATAGTTG | 171 |
| Promoter | TTTACTACTTGCTTGAGCCCCACCCTTGCTTATCGGTTGAACATTACA | |
| PVUBI2 (2) | AGATACACTGCGGGTGGCCTAAGGCACACCGTCCGAAACCGGCAAACC | |
| | AAGCCTGATCGCCGAAATCCAAAATCACTACCGGCAATCTCTAAAGTT | |
| | TATTTCATCCTTATATGACGAGGAAAGAAAAGAAGAGAGAAATAATAT | |
| | CTTAACTTCTAAATCAGTCGCGTCAACTTTCTCGGCTAAGAAAGTGAG | |
| | CACTATCATTTCGCAGACCATGTCATGAGTGCCGACTTGCCATATCTT | |
| | ATTATATTCTTATTTATTTAATTATAATCCCATTGCAATACGTCTATT | |
| | CTATCATGGCCTGCCACTAACGCTCCGTCTAACGTCGTTAAGCCATTG | |
| | TCATAAGCGGCTGCTCAAAACTCTTCCCGGTGGAGGCGAGGCGTTAAC | |
| | GGCGTCTACAAATCTAACGGCCACCAACCATCCAGCCGCCTCTCGAAA | |
| | GCTCCGCTCCGATCGCGGAAATTGCGTGGCGGAGACGAGCGGGCTCCT | |
| | CTCACACGGCCCGGAACCGTCACGGCACGGGTGGGGGATTCCTTCCCC | |
| | AACCCTCCCCACCTCTCCTCCCCCCGTCGCAGCCCATAAATACAGGGC | |
| | CCTCCGCGCCTCTTCCCACAATCTCACATCGTCTCATCGTTCGGAGCG | |
| | CACAACCCCCGGGTTCCAAATCCAAATTGCTCTTCTCGCGACCCTCGG | |
| | CGATCCTTCCCCCGCTTCAAGGTACGGCGATCGTCTCCCCCGTCCTCT | |
| | TGCCCCATCTCCTCGCTCGGCGTGGTTTGGTGGTTCTGCTTGGTCTGT | |
| | GGCTAGGAACTAGGCTGAGGCGTTGACGAAATCATGCTAGATCCGCGT | |
| | GTTTCCTGATCGTGGGTGGCTGGGAGGTGGGGTTTTCGTGTAGATCTG | |
| | ATCGGTTCCGCTGTTTATCCTGTCATGCTCATGTGATTTGTGGGGATT | |
| | TTAGGTCGTTTGTCCGGGAATCGTGGGGTTGCTTCTAGGCTGTTCGTA | |
| | GATGAGATCGTTCTCACGATCTGCTGGGTCGCTGCCTAGGTTCAGCTA | |
| | GGTCTGCCCTGTTTTTGGGTTCGTTTTCGGGATCTGTACGTGCATCTA | |
| | TTATCTGGTTCGATGGTGCTAGCTAGGAACAAACAACTGATTCGTCCG | |
| | ATCGATTGTTTTGTTGCCATGTGCAAGGTTAGGTCGTTATCTGATTGC | |
| | TGTAGATCAGAGTAGAATAAGATCATCACAAGCTAGCTCTTGGGCTTA | |
| | TTATGAATCTGCGTTTGTTGCATGATTAAGATGATTATGCTTTTTCTT | |
| | ATGCTGCCGTTTGTATATGATGCGGTAGCTTTTAACTGAATAGCACAC | |
| | CTTTCCTGTTTAGTTAGATTAGATTAGATTGCATGATAGATGAGGATA | |
| | TATGCTGCTACATCAGTTTGATGATTCTCTGGTACCTCATAATCAACT | |
| | AGCTCATGTGCTTAAATTGAAACTGCATGTGCCACATGATTAAGATGC | |
| | TAAGATTGGTGAAGATATATACGCTGCTGTTCCTATAGGATCCTGTAG | |
| | CTTTTACCTGGTCAACATGCATCGTCCTGTTATGGATAGATATGCATG | |
| | ATAGATGAAGATATGTACTGCTACAATTTGATGATTCTTTTGTGCACC | |
| | TGATGATCATGCATGCTCTTTGCCCTTACTTTGATATACTTGGATGAT | |
| | GGCATGCTTAGTACTAATGATGTGATGAACACACATGACCTGTTGGTA | |
| | TGAATATGATGTTGCTGTTTGCTTGTGATGAGTTCTGTTTGTTTACTG | |
| | CTAGGCACTTACCCTGTTGTCTGGTTCTCTTTTGCAG | |
| Promoter | TTCAAGCTTGGAGGAAGAACTCGAGAGGGAATTGCAGATCATGAGGCA | 75 |
| BdUbi10 | GATGGCTATTTTTGTGTCACATATGCGCAAAAAGAGAGGCTATATTTG | |
| | TGTCCCTAGGTTCTTCGTTGTATTGCAGTTTCCATATCAATCTGACTT | |
| | GGTCGCATGAGAAATTGATGGTTAAATAATTTGAATCTCTCATGTAGT | |
| | ATCAACTATTAGATATTATTTTCACCAAATATATTTCCATCGGAGAAG | |
| | AAGAGGCTACAGAGGAAGCAGAAGAGAGGGGTGGGAGAATTTTTACAC | |
| | TTTTGTACACCCACTTAAACAGCAAAATCCGTATGAAAACAGGCCCAC | |
| | CAAAACAATGCCACGATAACAATCCGTAGAAACAAAAGCTTCATTTAA | |
| | CAGCGGCGCAACAAAGCACGCTTATCCATGGTAGTTGTAGTCCGTATG | |
| | CGATCCAAAGATCACGATTCACGCGTGACGGACGGACGACGCGTGCCA | |
| | CACCACAACTAACGGCATCCATGGTAGTTGTAGTCCGTATGCGATCCA | |
| | AAGATCACGATTCACGCGTGACGGACGGACGACGCGCGCCACACCACA | |
| | ACTAACAGCGTGAGCCAGCGTCCAAACTCCGGATGGCAACGGGGACGA | |
| | AACCCGTCGGGTAGTCACTGCCCAAACCCGTCCCCGCAACCTTCATCC | |
| | CAAACCCGTCCCCGTTTCCGGTCGCGGGTTTCAGTTTTCTACCAGACC | |
| | CGTCCCCATCGGGTTTTTCATCCCCGTCGGGAAATCCGAACCCGCCAG | |
| | CATTTCAGCACCAAGCCAAAGTTGCAGCAGCAACATGAATAAAAAACA | |
| | ACCCGTTTCAACACCAAGATAAAACAAAACATTATAATTTAGACAACA | |
| | TTTCACACGTATAACAATAACATATAGTTCTCACATATAACAACACCA | |
| | TTTCACACATAAAACAACACCATTTGGGATAAAAATATGGGCTATATC | |
| | AGGCCATTTTTATGGGCCATATTGAGTTTTCGTGGGTTTCACAGGTAC | |
| | CGGATTTGTAGAATGCTGAACCGGGTTTGAACCGTAAAATCCGCGGGT | |
| | ATTGAATTTGACCCAATCCCGTCGTCCCCTGGTGGGGTAAAAACACCA | |
| | TCTTGAGTCCAAACGGCCACCAACCAAACTCCGACGGCAACAAACAAA | |
| | CGGCGTTGCTTTGCTCCTCGGTATCTCCGTGACCGCTCAATCTCCCGG | |
| | CTGTTTCCCCGGAATTGCGTGGACTCTCTCATCCACACGCAAACCGCC | |
| | TCTCCCTCCTCTCTCGTCCTATCCGCCCCGGTGCCGTAGCCTCACGGG | |
| | ACTCTTCTTCCTCCCTTGCTATAAAATCCCCGCCCCCTCCCGTCTCCT | |
| | CTCCACACATCCAAACTCTCAATCGCACCGAGAAAAATCTCCTAGCGA | |
| | TCGAAGCGAAGCCTCTCCCGATCCTCTCAAGGTACGCCCGTTTCCCGT | |
| | CGATCCTCCTCCTTCCGTTCGTGTTCTGTAGCCGATCGATTCGATTCC | |
| | CTTACACCCGTTCGTGTTCTCTCGTGGATCGATCGATTGTTTGTTGCT | |
| | AGAAGGAACTCGTAGATCTGGCGTTTATGAACTGTGATTCGGGTTAGT | |
| | CCAGATCGATTCAGGTCGGTCGTCGTTGAGCCTCTCGGCTATGTCTGG | |
| | ATTATCGTGTAGATCTGCTGGTTCAGTTGATTATGTTCTTCTAGGAGT | |
| | AATTTCGTTGGGTCAGCGCGATTTCTGCTTAATCTATGCTGCTTATTG | |
| | CGCCTGTACCTATCTACTAAGCTATGTGCACCTGTAATTTTGCTAGAT | |
| | TATTCGTTCATCCTCGTAGTTGGTTTGTCACAGTAATCCGTATGGGTT | |
| | CTGACGATGTTATTGTTGGTCATACCTAGGCTTCTCCAGATTTTATTT | |
| | TGTTAAAATTGGATAGATCTGCTACTGATAGTTGATGATGGAATTTGG | |
| | TGCTGAATCTATGCTATTTATTGCGCCTATACCTGATCTATCGGGCTA | |
| | TGTACGGCTGTAGTTTACTGGATTATTCGTTCATCCTCGGTAGTTGGT | |
| | TCATCGTTTGGGTTCTGACGATAATATTGTTGATTATGCGTAGGCTTC | |
| | TGCAGATTGTTGTTAAAATTGGATACATCGGTTACTGATGGTTGATGA | |
| | TAGATTTGTGCTGAACCTATCTGTTTATTGCTCCTATACCTGATCTAT | |
| | AGGGCTATGTATGCCTGTAATTTACCAGATTATTCGTTCATCCTCGTA | |
| | GTTGGTTCATCTCTATAATTCGTATGGGTTCTTATGATGTTATCGTTG | |
| | ATTATGCCTAGTCTTATACAGATTATTGTGTCAAGATTGAATATACCT | |
| | GCTACTGATCGGTGATAATTTGGTTAGTAGTTTGCAATCTGCTAGGAA | |
| | CACGTTACCACTGTAATCTGTAAACATGGTTTGCCAGAGTAGTTTGTT | |
| | CTACTACTCTTGATATGGTTGCTGATTTTAGTCGCCTCCTTTTGGATC | |
| | ATGTATTGATGTCCTTGCAGATTTCCGTGTACTTACCCCGGCTTTTGT | |
| | GTACTTCGTGTTAACAG | |
| PvUbi2 | GGAGGAAGCCAACTAAACAAGACCATAACCATGGTGACATTTGACATA | 134 |
| promoter | GTTGTTTACTACTTGCTTGAGCCCCACCCTTGCTTATCGGTTGAACAT | |
| | TACAAGATACACTGCGGGTGGCCTAAGGCACACCGTCCGAAACCGGCA | |
| | AACCAAGCCTGATCGCCGAAATCCAAAATCACTACCGGCAATCTCTAA | |
| | AGTTTATTTCATCCTTATATGACGAGGAAAGAAAAGAAGAGAGAAATA | |
| | ATATCTTAACTTCTAAATCAGTCGCGTCAACTTTCTCGGCTAAGAAAG | |
| | TGAGCACTATCATTTCGCAGACCATGTCATGAGTGCCGACTTGCCATA | |
| | TCTTATTATATTCTTATTTATTTAATTATAATCCCATTGCAATACGTC | |
| | TATTCTATCATGGCCTGCCACTAACGCTCCGTCTAACGTCGTTAAGCC | |
| | ATTGTCATAAGCGGCTGCTCAAAACTCTTCCCGGTGGAGGCGAGGCGT | |
| | TAACGGCGTCTACAAATCTAACGGCCACCAACCATCCAGCCGCCTCTC | |
| | GAAAGCTCCGCTCCGATCGCGGAAATTGCGTGGCGGAGACGAGCGGGC | |
| | TCCTCTCACACGGCCCGGAACCGTCACGGCACGGGTGGGGGATTCCTT | |
| | CCCCAACCCTCCCCACCTCTCCTCCCCCCGTCGCAGCCCATAAATACA | |
| | GGGCCCTCCGCGCCTCTTCCCACAATCTCACATCGTCTCATCGTTCGG | |
| | AGCGCACAACCCCCGGGTTCCAAATCCAAATTGCTCTTCTCGCGACCC | |
| | TCGGCGATCCTTCCCCCGCTTCAAGGTACGGCGATCGTCTCCCCCGTC | |
| | CTCTTGCCCCATCTCCTCGCTCGGCGTGGTTTGGTGGTTCTGCTTGGT | |
| | CTGTGGCTAGGAACTAGGCTGAGGCGTTGACGAAATCATGCTAGATCC | |
| | GCGTGTTTCCTGATCGTGGGTGGCTGGGAGGTGGGGTTTTCGTGTAGA | |
| | TCTGATCGGTTCCGCTGTTTATCCTGTCATGCTCATGTGATTTGTGGG | |
| | GATTTTAGGTCGTTTGTCCGGGAATCGTGGGGTTGCTTCTAGGCTGTT | |
| | CGTAGATGAGATCGTTCTCACGATCTGCTGGGTCGCTGCCTAGGTTCA | |
| | GCTAGGTCTGCCCTGTTTTTGGGTTCGTTTTCGGGATCTGTACGTGCA | |
| | TCTATTATCTGGTTCGATGGTGCTAGCTAGGAACAAACAACTGATTCG | |
| | TCCGATCGATTGTTTTGTTGCCATGTGCAAGGTTAGGTCGTTATCTGA | |
| | TTGCTGTAGATCAGAGTAGAATAAGATCATCACAAGCTAGCTCTTGGG | |
| | CTTATTATGAATCTGCGTTTGTTGCATGATTAAGATGATTATGCTTTT | |
| | TCTTATGCTGCCGTTTGTATATGATGCGGTAGCTTTTAACTGAATAGC | |
| | ACACCTTTCCTGTTTAGTTAGATTAGATTAGATTGCATGATAGATGAG | |
| | GATATATGCTGCTACATCAGTTTGATGATTCTCTGGTACCTCATAATC | |
| | AACTAGCTCATGTGCTTAAATTGAAACTGCATGTGCCACATGATTAAG | |
| | ATGCTAAGATTGGTGAAGATATATACGCTGCTGTTCCTATAGGATCCT | |
| | GTAGCTTTTACCTGGTCAACATGCATCGTCCTGTTATGGATAGATATG | |
| | CATGATAGATGAAGATATGTACTGCTACAATTTGATGATTCTTTTGTG | |
| | CACCTGATGATCATGCATGCTCTTTGCCCTTACTTTGATATACTTGGA | |
| | TGATGGCATGCTTAGTACTAATGATGTGATGAACACACATGACCTGTT | |
| | GGTATGAATATGATGTTGCTGTTTGCTTGTGATGAGTTCTGTTTGTTT | |
| | ACTGCTAGGCACTTACCCTGTTGTCTGGTTCTCTTTTGCAGA | |
| PvUbi2 | GAAGCCAACTAAACAAGACCATAACCATGGTGACATTTGACATAGTTG | 172 |
| promoter (2) | TTTACTACTTGCTTGAGCCCCACCCTTGCTTATCGGTTGAACATTACA | |
| | AGATACACTGCGGGTGGCCTAAGGCACACCGTCCGAAACCGGCAAACC | |
| | AAGCCTGATCGCCGAAATCCAAAATCACTACCGGCAATCTCTAAAGTT | |
| | TATTTCATCCTTATATGACGAGGAAAGAAAAGAAGAGAGAAATAATAT | |
| | CTTAACTTCTAAATCAGTCGCGTCAACTTTCTCGGCTAAGAAAGTGAG | |
| | CACTATCATTTCGCAGACCATGTCATGAGTGCCGACTTGCCATATCTT | |
| | ATTATATTCTTATTTATTTAATTATAATCCCATTGCAATACGTCTATT | |
| | CTATCATGGCCTGCCACTAACGCTCCGTCTAACGTCGTTAAGCCATTG | |
| | TCATAAGCGGCTGCTCAAAACTCTTCCCGGTGGAGGCGAGGCGTTAAC | |
| | GGCGTCTACAAATCTAACGGCCACCAACCATCCAGCCGCCTCTCGAAA | |
| | GCTCCGCTCCGATCGCGGAAATTGCGTGGCGGAGACGAGCGGGCTCCT | |
| | CTCACACGGCCCGGAACCGTCACGGCACGGGTGGGGGATTCCTTCCCC | |
| | AACCCTCCCCACCTCTCCTCCCCCCGTCGCAGCCCATAAATACAGGGC | |
| | CCTCCGCGCCTCTTCCCACAATCTCACATCGTCTCATCGTTCGGAGCG | |
| | CACAACCCCCGGGTTCCAAATCCAAATTGCTCTTCTCGCGACCCTCGG | |
| | CGATCCTTCCCCCGCTTCAAGGTACGGCGATCGTCTCCCCCGTCCTCT | |
| | TGCCCCATCTCCTCGCTCGGCGTGGTTTGGTGGTTCTGCTTGGTCTGT | |
| | GGCTAGGAACTAGGCTGAGGCGTTGACGAAATCATGCTAGATCCGCGT | |
| | GTTTCCTGATCGTGGGTGGCTGGGAGGTGGGGTTTTCGTGTAGATCTG | |
| | ATCGGTTCCGCTGTTTATCCTGTCATGCTCATGTGATTTGTGGGGATT | |
| | TTAGGTCGTTTGTCCGGGAATCGTGGGGTTGCTTCTAGGCTGTTCGTA | |
| | GATGAGATCGTTCTCACGATCTGCTGGGTCGCTGCCTAGGTTCAGCTA | |
| | GGTCTGCCCTGTTTTTGGGTTCGTTTTCGGGATCTGTACGTGCATCTA | |
| | TTATCTGGTTCGATGGTGCTAGCTAGGAACAAACAACTGATTCGTCCG | |
| | ATCGATTGTTTTGTTGCCATGTGCAAGGTTAGGTCGTTATCTGATTGC | |
| | TGTAGATCAGAGTAGAATAAGATCATCACAAGCTAGCTCTTGGGCTTA | |
| | TTATGAATCTGCGTTTGTTGCATGATTAAGATGATTATGCTTTTTCTT | |
| | ATGCTGCCGTTTGTATATGATGCGGTAGCTTTTAACTGAATAGCACAC | |
| | CTTTCCTGTTTAGTTAGATTAGATTAGATTGCATGATAGATGAGGATA | |
| | TATGCTGCTACATCAGTTTGATGATTCTCTGGTACCTCATAATCAACT | |
| | AGCTCATGTGCTTAAATTGAAACTGCATGTGCCACATGATTAAGATGC | |
| | TAAGATTGGTGAAGATATATACGCTGCTGTTCCTATAGGATCCTGTAG | |
| | CTTTTACCTGGTCAACATGCATCGTCCTGTTATGGATAGATATGCATG | |
| | ATAGATGAAGATATGTACTGCTACAATTTGATGATTCTTTTGTGCACC | |
| | TGATGATCATGCATGCTCTTTGCCCTTACTTTGATATACTTGGATGAT | |
| | GGCATGCTTAGTACTAATGATGTGATGAACACACATGACCTGTTGGTA | |
| | TGAATATGATGTTGCTGTTTGCTTGTGATGAGTTCTGTTTGTTTACTG | |
| | CTAGGCACTTACCCTGTTGTCTGGTTCTCTTTTGCAG | |
| ZmUbi | CTGCAGTGCAGCGTGACCCGGTCGTGCCCCTCTCTAGAGATAATGAGC | 135 |
| promoter | ATTGCATGTCTAAGTTATAAAAAATTACCACATATTTTTTTTGTCACA | |
| | CTTGTTTGAAGTGCAGTTTATCTATCTTTATACATATATTTAAACTTT | |
| | ACTCTACGAATAATATAATCTATAGTACTACAATAATATCAGTGTTTT | |
| | AGAGAATCATATAAATGAACAGTTAGACATGGTCTAAAGGACAATTGA | |
| | GTATTTTGACAACAGGACTCTACAGTTTTATCTTTTTAGTGTGCATGT | |
| | GTTCTCCTTTTTTTTTGCAAATAGCTTCACCTATATAATACTTCATCC | |
| | ATTTTATTAGTACATCCATTTAGGGTTTAGGGTTAATGGTTTTTATAG | |
| | ACTAATTTTTTTAGTACATCTATTTTATTCTATTTTAGCCTCTAAATT | |
| | AAGAAAACTAAAACTCTATTTTAGTTTTTTTATTTAATAATTTAGATA | |
| | TAAAATAGAATAAAATAAAGTGACTAAAAATTAAACAAATACCCTTTA | |
| | AGAAATTAAAAAAACTAAGGAAACATTTTTCTTGTTTCGAGTAGATAA | |
| | TGCCAGCCTGTTAAACGCCGTCGACGAGTCTAACGGACACCAACCAGC | |
| | GAACCAGCAGCGTCGCGTCGGGCCAAGCGAAGCAGACGGCACGGCATC | |
| | TCTGTCGCTGCCTCTGGACCCCTCTCGAGAGTTCCGCTCCACCGTTGG | |
| | ACTTGCTCCGCTGTCGGCATCCAGAAATTGCGTGGCGGAGCGGCAGAC | |
| | GTGAGCCGGCACGGCAGGCGGCCTCCTCCTCCTCTCACGGCACCGGCA | |
| | GCTACGGGGGATTCCTTTCCCACCGCTCCTTCGCTTTCCCTTCCTCGC | |
| | CCGCCGTAATAAATAGACACCCCCTCCACACCCTCTTTCCCCAACCTC | |
| | GTGTTGTTCGGAGCGCACACACACACAACCAGATCTCCCCCAAATCCA | |
| | CCCGTCGGCACCTCCGCTTCAAGGTACGCCGCTCGTCCTCCCCCCCCC | |
| | CCCTCTCTACCTTCTCTAGATCGGCGTTCCGGTCCATGGTTAGGGCCC | |
| | GGTAGTTCTACTTCTGTTCATGTTTGTGTTAGATCCGTGTTTGTGTTA | |
| | GATCCGTGCTGCTAGCGTTCGTACACGGATGCGACCTGTACGTCAGAC | |
| | ACGTTCTGATTGCTAACTTGCCAGTGTTTCTCTTTGGGGAATCCTGGG | |
| | ATGGCTCTAGCCGTTCCGCAGACGGGATCGATTTCATGATTTTTTTTG | |
| | TTTCGTTGCATAGGGTTTGGTTTGCCCTTTTCCTTTATTTCAATATAT | |
| | GCCGTGCACTTGTTTGTCGGGTCATCTTTTCATGCTTTTTTTTGTCTT | |
| | GGTTGTGATGATGTGGTCTGGTTGGGCGGTCGTTCTAGATCGGAGTAG | |
| | AATTCTGTTTCAAACTACCTGGTGGATTTATTAATTTTGGATCTGTAT | |
| | GTGTGTGCCATACATATTCATAGTTACGAATTGAAGATGATGGATGGA | |
| | AATATCGATCTAGGATAGGTATACATGTTGATGCGGGTTTTACTGATG | |
| | CATATACAGAGATGCTTTTTGTTCGCTTGGTTGTGATGATGTGGTGTG | |
| | GTTGGGCGGTCGTTCATTCGTTCTAGATCGGAGTAGAATACTGTTTCA | |
| | AACTACCTGGTGTATTTATTAATTTTGGAACTGTATGTGTGTGTCATA | |
| | CATCTTCATAGTTACGAGTTTAAGATGGATGGAAATATCGATCTAGGA | |
| | TAGGTATACATGTTGATGTGGGTTTTACTGATGCATATACATGATGGC | |
| | ATATGCAGCATCTATTCATATGCTCTAACCTTGAGTACCTATCTATTA | |
| | TAATAAACAAGTATGTTTTATAATTATTTTGATCTTGATATACTTGGA | |
| | TGATGGCATATGCAGCAGCTATATGTGGATTTTTTTAGCCCTGCCTTC | |
| | ATACGCTATTTATTTGCTTGGTACTGTTTCTTTTGTCGATGCTCACCC | |
| | TGTTGTTTGGTGTTACTTCTGCAG | |
| BdUbi10 | GAAGAACTCGAGAGGGAATTGCAGATCATGAGGCAGATGGCTATTTTT | 136 |
| promoter | GTGTCACATATGCGCAAAAAGAGAGGCTATATTTGTGTCCCTAGGTTC | |
| | TTCGTTGTATTGCAGTTTCCATATCAATCTGACTTGGTCGCATGAGAA | |
| | ATTGATGGTTAAATAATTTGAATCTCTCATGTAGTATCAACTATTAGA | |
| | TATTATTTTCACCAAATATATTTCCATCGGAGAAGAAGAGGCTACAGA | |
| | GGAAGCAGAAGAGAGGGGTGGGAGAATTTTTACACTTTTGTACACCCA | |
| | CTTAAACAGCAAAATCCGTATGAAAACAGGCCCACCAAAACAATGCCA | |
| | CGATAACAATCCGTAGAAACAAAAGCTTCATTTAACAGCGGCGCAACA | |
| | AAGCACGCTTATCCATGGTAGTTGTAGTCCGTATGCGATCCAAAGATC | |
| | ACGATTCACGCGTGACGGACGGACGACGCGTGCCACACCACAACTAAC | |
| | GGCATCCATGGTAGTTGTAGTCCGTATGCGATCCAAAGATCACGATTC | |
| | ACGCGTGACGGACGGACGACGCGCGCCACACCACAACTAACAGCGTGA | |
| | GCCAGCGTCCAAACTCCGGATGGCAACGGGGACGAAACCCGTCGGGTA | |
| | GTCACTGCCCAAACCCGTCCCCGCAACCTTCATCCCAAACCCGTCCCC | |
| | GTTTCCGGTCGCGGGTTTCAGTTTTCTACCAGACCCGTCCCCATCGGG | |
| | TTTTTCATCCCCGTCGGGAAATCCGAACCCGCCAGCATTTCAGCACCA | |
| | AGCCAAAGTTGCAGCAGCAACATGAATAAAAAACAACCCGTTTCAACA | |
| | CCAAGATAAAACAAAACATTATAATTTAGACAACATTTCACACGTATA | |
| | ACAATAACATATAGTTCTCACATATAACAACACCATTTCACACATAAA | |
| | ACAACACCATTTGGGATAAAAATATGGGCTATATCAGGCCATTTTTAT | |
| | GGGCCATATTGAGTTTTCGTGGGTTTCACAGGTACCGGATTTGTAGAA | |
| | TGCTGAACCGGGTTTGAACCGTAAAATCCGCGGGTATTGAATTTGACC | |
| | CAATCCCGTCGTCCCCTGGTGGGGTAAAAACACCATCTTGAGTCCAAA | |
| | CGGCCACCAACCAAACTCCGACGGCAACAAACAAACGGCGTTGCTTTG | |
| | CTCCTCGGTATCTCCGTGACCGCTCAATCTCCCGGCTGTTTCCCCGGA | |
| | ATTGCGTGGACTCTCTCATCCACACGCAAACCGCCTCTCCCTCCTCTC | |
| | TCGTCCTATCCGCCCCGGTGCCGTAGCCTCACGGGACTCTTCTTCCTC | |
| | CCTTGCTATAAAATCCCCGCCCCCTCCCGTCTCCTCTCCACACATCCA | |
| | AACTCTCAATCGCACCGAGAAAAATCTCCTAGCGATCGAAGCGAAGCC | |
| | TCTCCCGATCCTCTCAAGGTACGCCCGTTTCCCGTCGATCCTCCTCCT | |
| | TCCGTTCGTGTTCTGTAGCCGATCGATTCGATTCCCTTACACCCGTTC | |
| | GTGTTCTCTCGTGGATCGATCGATTGTTTGTTGCTAGAAGGAACTCGT | |
| | AGATCTGGCGTTTATGAACTGTGATTCGGGTTAGTCCAGATCGATTCA | |
| | GGTCGGTCGTCGTTGAGCCTCTCGGCTATGTCTGGATTATCGTGTAGA | |
| | TCTGCTGGTTCAGTTGATTATGTTCTTCTAGGAGTAATTTCGTTGGGT | |
| | CAGCGCGATTTCTGCTTAATCTATGCTGCTTATTGCGCCTGTACCTAT | |
| | CTACTAAGCTATGTGCACCTGTAATTTTGCTAGATTATTCGTTCATCC | |
| | TCGTAGTTGGTTTGTCACAGTAATCCGTATGGGTTCTGACGATGTTAT | |
| | TGTTGGTCATACCTAGGCTTCTCCAGATTTTATTTTGTTAAAATTGGA | |
| | TAGATCTGCTACTGATAGTTGATGATGGAATTTGGTGCTGAATCTATG | |
| | CTATTTATTGCGCCTATACCTGATCTATCGGGCTATGTACGGCTGTAG | |
| | TTTACTGGATTATTCGTTCATCCTCGGTAGTTGGTTCATCGTTTGGGT | |
| | TCTGACGATAATATTGTTGATTATGCGTAGGCTTCTGCAGATTGTTGT | |
| | TAAAATTGGATACATCGGTTACTGATGGTTGATGATAGATTTGTGCTG | |
| | AACCTATCTGTTTATTGCTCCTATACCTGATCTATAGGGCTATGTATG | |
| | CCTGTAATTTACCAGATTATTCGTTCATCCTCGTAGTTGGTTCATCTC | |
| | TATAATTCGTATGGGTTCTTATGATGTTATCGTTGATTATGCCTAGTC | |
| | TTATACAGATTATTGTGTCAAGATTGAATATACCTGCTACTGATCGGT | |
| | GATAATTTGGTTAGTAGTTTGCAATCTGCTAGGAACACGTTACCACTG | |
| | TAATCTGTAAACATGGTTTGCCAGAGTAGTTTGTTCTACTACTCTTGA | |
| | TATGGTTGCTGATTTTAGTCGCCTCCTTTTGGATCATGTATTGATGTC | |
| | CTTGCAGATTTCCGTGTACTTACCCCGGCTTTTGTGTACTTCGTGTTA | |
| | ACAG | |
| Short 35S | GGAGGTCAACATGGTGGAGCACGACACTCTGGTCTACTCCAAAAATGT | 137 |
| promoter | CAAAGATACAGTCTCAGAAGATCAAAGGGCTATTGAGACTTTTCAACA | |
| | AAGGATAATTTCGGGAAACCTCCTCGGATTCCATTGCCCAGCTATCTG | |
| | TCACTTCATCGAAAGGACAGTAGAAAAGGAAGGTGGCTCCTACAAATG | |
| | CCATCATTGCGATAAAGGAAAGGCTATCATTCAAGATCTCTCTGCCGA | |
| | CAGTGGTCCCAAAGATGGACCCCCACCCACGAGGAGCATCGTGGAAAA | |
| | AGAAGAGGTTCCAACCACGTCTACAAAGCAAGTGGATTGATGTGACAT | |
| | CTCCACTGACGTAAGGGATGACGCACAATCCCACTATCCTTCGCAAGA | |
| | CCCTTCCTCTATATAAGGAAGTTCATTTCATTTGGAGAGGACACGC | |
| Short 35S | GTCAACATGGTGGAGCACGACACTCTGGTCTACTCCAAAAATGTCAAA | 173 |
| promoter (2) | GATACAGTCTCAGAAGATCAAAGGGCTATTGAGACTTTTCAACAAAGG | |
| | ATAATTTCGGGAAACCTCCTCGGATTCCATTGCCCAGCTATCTGTCAC | |
| | TTCATCGAAAGGACAGTAGAAAAGGAAGGTGGCTCCTACAAATGCCAT | |
| | CATTGCGATAAAGGAAAGGCTATCATTCAAGATCTCTCTGCCGACAGT | |
| | GGTCCCAAAGATGGACCCCCACCCACGAGGAGCATCGTGGAAAAAGAA | |
| | GAGGTTCCAACCACGTCTACAAAGCAAGTGGATTGATGTGACATCTCC | |
| | ACTGACGTAAGGGATGACGCACAATCCCACTATCCTTCGCAAGACCCT | |
| | TCCTCTATATAAGGAAGTTCATTTCATTTGGAGAGGACACGC | |
| 2x 35S + | GGAGGTCAACATGGTGGAGCACGACACTCTGGTCTACTCCAAAAATGT | 138 |
| TMV | CAAAGATACAGTCTCAGAAGATCAAAGGGCTATTGAGACTTTTCAACA | |
| 5′UTR | AAGGATAATTTCGGGAAACCTCCTCGGATTCCATTGCCCAGCTATCTG | |
| promoter | TCACTTCATCGAAAGGACAGTAGAAAAGGAAGGTGGCTCCTACAAATG | |
| | CCATCATTGCGATAAAGGAAAGGCTATCATTCAAGATCTCTCTGCCGA | |
| | CAGTGGTCCCAAAGATGGACCCCCACCCACGAGGAGCATCGTGGAAAA | |
| | AGAAGAGGTTCCAACCACGTCTACAAAGCAAGTGGATTGATGTGATAA | |
| | CATGGTGGAGCACGACACTCTGGTCTACTCCAAAAATGTCAAAGATAC | |
| | AGTCTCAGAAGATCAAAGGGCTATTGAGACTTTTCAACAAAGGATAAT | |
| | TTCGGGAAACCTCCTCGGATTCCATTGCCCAGCTATCTGTCACTTCAT | |
| | CGAAAGGACAGTAGAAAAGGAAGGTGGCTCCTACAAATGCCATCATTG | |
| | CGATAAAGGAAAGGCTATCATTCAAGATCTCTCTGCCGACAGTGGTCC | |
| | CAAAGATGGACCCCCACCCACGAGGAGCATCGTGGAAAAAGAAGAGGT | |
| | TCCAACCACGTCTACAAAGCAAGTGGATTGATGTGACATCTCCACTGA | |
| | CGTAAGGGATGACGCACAATCCCACTATCCTTCGCAAGACCCTTCCTC | |
| | TATATAAGGAAGTTCATTTCATTTGGAGAGGACACGCTCGAGTATAAG | |
| | AGCTCATTTTTACAACAATTACCAACAACAACAAACAACAAACAACAT | |
| | TACAATTACATTTACAATTATCGATACA | |
| 2x 35S + | GGAGGTCAACATGGTGGAGCACGACACTCTGGTCTACTCCAAAAATGT | 174 |
| TMV | CAAAGATACAGTCTCAGAAGATCAAAGGGCTATTGAGACTTTTCAACA | |
| 5′UTR | AAGGATAATTTCGGGAAACCTCCTCGGATTCCATTGCCCAGCTATCTG | |
| promoter (2) | TCACTTCATCGAAAGGACAGTAGAAAAGGAAGGTGGCTCCTACAAATG | |
| | CCATCATTGCGATAAAGGAAAGGCTATCATTCAAGATCTCTCTGCCGA | |
| | CAGTGGTCCCAAAGATGGACCCCCACCCACGAGGAGCATCGTGGAAAA | |
| | AGAAGAGGTTCCAACCACGTCTACAAAGCAAGTGGATTGATGTGATAA | |
| | CATGGTGGAGCACGACACTCTGGTCTACTCCAAAAATGTCAAAGATAC | |
| | AGTCTCAGAAGATCAAAGGGCTATTGAGACTTTTCAACAAAGGATAAT | |
| | TTCGGGAAACCTCCTCGGATTCCATTGCCCAGCTATCTGTCACTTCAT | |
| | CGAAAGGACAGTAGAAAAGGAAGGTGGCTCCTACAAATGCCATCATTG | |
| | CGATAAAGGAAAGGCTATCATTCAAGATCTCTCTGCCGACAGTGGTCC | |
| | CAAAGATGGACCCCCACCCACGAGGAGCATCGTGGAAAAAGAAGAGGT | |
| | TCCAACCACGTCTACAAAGCAAGTGGATTGATGTGACATCTCCACTGA | |
| | CGTAAGGGATGACGCACAATCCCACTATCCTTCGCAAGACCCTTCCTC | |
| | TATATAAGGAAGTTCATTTCATTTGGAGAGGACACGCTCGAGTATAAG | |
| | AGCTCATTTTTACAACAATTACCAACAACAACAAACAACAAACAACAT | |
| | TACAATTACATTTACAATTATCGATAC | |
| CsVMV | CCAGAAGGTAATTATCCAAGATGTAGCATCAAGAATCCAATGTTTACG | 139 |
| promoter | GGAAAAACTATGGAAGTATTATGTAAGCTCAGCAAGAAGCAGATCAAT | |
| | ATGCGGCACATATGCAACCTATGTTCAAAAATGAAGAATGTACAGATA | |
| | CAAGATCCTATACTGCCAGAATACGAAGAAGAATACGTAGAAATTGAA | |
| | AAAGAAGAACCAGGCGAAGAAAAGAATCTTGATGACGTAAGCACTGAC | |
| | GACAACAATGAAAAGAAGAAGATAAGGTCGGTGATTGTGAAAGAGACA | |
| | TAGAGGACACATGTAAGGTGGAAAATGTAAGGGCGGAAAGTAACCTTA | |
| | TCACAAAGGAATCTTATCCCCCACTACTTATCCTTTTATATTTTTCCG | |
| | TGTCATTTTTGCCCTTGAGTTTTCCTATATAAGGAACCAAGTTCGGCA | |
| | TTTGTGAAAACAAGAAAAAATTTGGTGTAAGCTATTTTCTTTGAAGTA | |
| | CTGAGGATACAACTTCAGAGAAATTTGTAAGTTTGT | |

The term “constitutive” promoter refers to a nucleotide sequence that, when operably linked with a nucleic acid encoding a polypeptide (e.g., a metabolic polypeptide), causes RNA to be transcribed from the nucleic acid in a cell under most or all physiological conditions. In some embodiments, a suitable plant specific constitutive promoter may comprise but is not limited to: a Zea mays Ubiquitin 1 promoter (ZmUbi), an Oryza sativa Actin 1 promoter (OsAc1), a Panicum virgatum L. Ubiquitin 2 promoter (PvUbi2), a Panicum virgatum L. Ubiquitin 1 fusion promoter (PvUbi1+3), an Oryza sativa Cytochrome c gene promoter (OsCc1), an Epipremnum aureum Ubiquitin promoter, an Epipremnum aureum Actin promoter, an Epipremnum aureum Histone H3 promoter, a Cauliflower Mosaic virus promoter (2×CaMV35S), a Agrobacterium tumefaciens Nopaline synthase gene promoter (NOS), an Epipremnum aureum ribulose bisphosphate carboxylase/oxygenase activase 2 promoter, an Epipremnum aureum Metallothionein-like protein type 3 promoter, an Epipremnum aureum abscisic stress-ripening protein 2-like promoter, an Epipremnum aureum RNA-binding protein cabeza-like promoter, or a combination of any characteristic portion of any one or more of these promoters.

### Terminator and Polyadenylation Sequences

In some embodiments, a construct described herein comprises a terminator. The term “terminator” refers to a DNA sequence recognized by enzymes/polypeptides that can terminate and/or end transcription of a gene or operon. For example, a terminator typically refers to, e.g., a nucleotide sequence in the DNA, that induced the release of newly synthetized transcript RNA from the transcriptional complex. This frees the RNA polymerase and associated factors related to the transcription machinery. Thus, in some embodiments, a construct comprises one of the non-limiting example terminators described herein. In some embodiments, a terminator described herein is operably linked to a coding region.

In some embodiments, a terminator can code for a 3′UTR and/or a Polyadenylation signal in the mRNA transcript. In some embodiments, a terminator can be a plant cell terminator, a viral terminator, a chimeric terminator, an engineered terminator, a tissue-specific terminator, or other types of terminator known in the art.

In some embodiments, a terminator is one listed herein as included in Table 4. In some embodiments, a terminator sequence is at least 85%, at least 90%, at least 95%, at least 98% or at least 99% identical to a sequence included in Table 4. In some embodiments, a terminator sequence is a characteristic portion of a sequence included in Table 4.

TABLE 4
Exemplary Terminator Polynucleotide Sequences

| | | SEQ ID |
|---|---|---|
| Description | Sequence | NO: |
| Cauliflower | AGCTTCTCTAGCTAGAGTCGATCGACAAGCTCGAGTTTCTCCATAATA | 76 |
| Mosaic | ATGTGTGAGTAGTTCCCAGATAAGGGAATTAGGGTTCCTATAGGGTTT | |
| virus 35S | CGCTCATGTGTTGAGCATATAAGAAACCCTTAGTATGTATTTGTATTT | |
| terminator | GTAAAATACTTCTATCAATAAAATTTCTAATTCCTAAAACCAAAATCC | |
| (TerCaMV3 | AGTACTAAAATCCAGAT | |
| 5S) | | |
| Arabidopsis | AGCTTGCTCTCAAGATCAAAGGCTTAAAAAGCTGGGGTTTTATGAATG | 77 |
| thaliana | GGATCAAAGTTTCTTTTTTTCTTTTATATTTGCTTCTCCATTTGTTTG | |
| Actin 2 | TTTCATTTCCCTTTTTGTTTTCGTTTCTATGATGCACTTGTGTGTGAC | |
| terminator | AAACTCTCTGGGTTTTTACTTACGTCTGCGTTTCAAAAAAAAAAACCG | |
| (TerAthAct | CTTTCGTTTTGCGTTTTAGTCCCATTGTTTTGTAGCTCTGAGTGATCG | |
| 2) | AATTGATGCCTCTTTATTCCTTTTGTTCCCTATAATTTCTTTCAAAAC | |
| | TCAGAAGAAAAACCTTGAAACTCTTTGCAATGTTAATATAAGTATTGT | |
| | ATAAGATTTTTATTGATTTGGTTATTAGTCTTACTTTTGCTACCTCCA | |
| | TCTTCACTTGGAACTGATATTCTGAATAGTTAAAGCGTTACATGTGTT | |
| | CCATTCACAAATGAACTTAAACTAGCACAAAGTCAGATATTTTAAGAT | |
| | CGCACCATTT | |
| Solanum | AGCTTTTATGTTGGTGATATGGTGGTAAATGTAGGGATTTAGTTTACA | 78 |
| lycopersicum | ATTGCGTATGTCTGTGTTGGATATCTGTAGTGCTGTTCTTATGGCTTA | |
| Histone | GATCTTGTAATTTCTCATTACAGTATCAATGAATAGATATCAGTTTCT | |
| H4 | AGTGATGACATTGGTTCGTCTTTTAGCTGTTGATTAATTTTTCTTAAT | |
| terminator | TGATTCATCCTATTGCAATTCTTCTGAATTTAAATTGTATACTGTGAA | |
| (TerSIHisH | ATTAAGAAAATTCTTGAAATTAATGAGAATTTGAGTAATAG | |
| 4) | | |
| Solanum | TTATGTTGGTGATATGGTGGTAAATGTAGGGATTTAGTTTACAATTGC | 175 |
| lycopersicum | GTATGTCTGTGTTGGATATCTGTAGTGCTGTTCTTATGGCTTAGATCT | |
| Histone | TGTAATTTCTCATTACAGTATCAATGAATAGATATCAGTTTCTAGTGA | |
| H4 | TGACATTGGTTCGTCTTTTAGCTGTTGATTAATTTTTCTTAATTGATT | |
| terminator | CATCCTATTGCAATTCTTCTGAATTTAAATTGTATACTGTGAAATTAA | |
| (TerSIHisH | GAAAATTCTTGAAATTAATGAGAATTTGAGTAATAG | |
| 4) (2) | | |
| Agrobacterium | AGCTTCTCTAGCTAGAGTCGATCGACAAGCTCGAGTTTCTCCATAATA | 79 |
| tumefaciens | ATGTGTGAGTAGTTCCCAGATAAGGGAATTAGGGTTCCTATAGGGTTT | |
| nopaline | CGCTCATGTGTTGAGCATATAAGAAACCCTTAGTATGTATTTGTATTT | |
| synthase | GTAAAATACTTCTATCAATAAAATTTCTAATTCCTAAAACCAAAATCC | |
| terminator | AGTACTAAAATCCAGAT | |
| (TerNos) | | |
| Agrobacterium | GTCAAGCAGATCGTTCAAACATTTGGCAATAAAGTTTCTTAAGATTGA | 176 |
| tumefaciens | ATCCTGTTGCCGGTCTTGCGATGATTATCATATAATTTCTGTTGAATT | |
| nopaline | ACGTTAAGCATGTAATAATTAACATGTAATGCATGACGTTATTTATGA | |
| synthase | GATGGGTTTTTATGATTAGAGTCCCGCAATTATACATTTAATACGCGA | |
| terminator | TAGAAAACAAAATATAGCGCGCAAACTAGGATAAATTATCGCGCGCGG | |
| (TerNos) (2) | TGTCATCTATGTTACTAGATCGA | |
| Agrobacterium | AGCTTGTCCTGCTTTAATGAGATATGCGAGAAGCCTATGATCGCATGA | 80 |
| tumefaciens | TATTTGCTTTCAATTCTGTTGTGCACGTTGTAAAAAACCTGAGCATGT | |
| octopine | GTAGCTCAGATCCTTACCGCCGGTTTCGGTTCATTCTAATGAATATAT | |
| synthase | CACCCGTTACTATCGTATTTTTATGAATAATATTCTCCGTTCAATTTA | |
| terminator | CTGATTGTACCCTACTACTTATATGTACAATATTAAAATGAAAACAAT | |
| (TerOcs) | ATATTGTGCTGAATAGGTTTATAGCGACATCTATGATAGAGCGCCACA | |
| | ATAACAAACAATTGCGTTTTATTATTACAAATCCAATTTTAAAAAAAG | |
| | CGGCAGAACCGGTCAAACCTAAAAGACTGATTACATAAATCTTATTCA | |
| | AATTTCAAAAGTGCCCCAGGGGCTAGTATCTACGACACACCGAGCGGC | |
| | GAACTAATAACGCTCACTGAAGGGAACTCCGGTTCCCCGCCGGCGCGC | |
| | ATGGGTGAGATTCCTTGAAGTTGAGTATTGGCCGTCCGCTCTACCGAA | |
| | AGTTACGGGCACCATTCAACCCGGTCCAGCACGGCGGCCGGGTAACCG | |
| | ACTTGCTGCCCCGAGAATTATGCAGCATTTTTTTGGTGTATGTGGGCC | |
| | CCAAATGAAGTGCAGGTCAAACCTTGACAGTGACGACAAATCGTTGGG | |
| | CGGGTCCAGGGCGAATTTTGCGACAACATGTCGAGGCTCAGCAGGACC | |
| | GCTTGAGACCACGAA | |
| Agrobacterium | GGACTCCCATGTTGGCAAAGGCAACCAAACAAACAATGAATGATCCGC | 177 |
| tumefaciens | TCCTGCATATGGGGCGGTTTGAGTATTTCAACTGCCATTTGGGCTGAA | |
| mannopine | TTGTAGACATGCTCCTGTCAGAAATTCCGTGATCTTACTCAATATTCA | |
| synthase | GTAATCTCGGCCAATATCCTAAATGTGCGTGGCTTTATCTGTCTTTGT | |
| terminator | ATTGTTTCATCAATTCATGTAACGTTTGCTTTTCTTATGAATTTTCAA | |
| (TerMas) | ATAAATTATC | |
| (2) | | |
| Agrobacterium | AGCTTGGACTCCCATGTTGGCAAAGGCAACCAAACAAACAATGAATGA | 81 |
| tumefaciens | TCCGCTCCTGCATATGGGGCGGTTTGAGTATTTCAACTGCCATTTGGG | |
| mannopine | CTGAATTGTAGACATGCTCCTGTCAGAAATTCCGTGATCTTACTCAAT | |
| synthase | ATTCAGTAATCTCGGCCAATATCCTAAATGTGCGTGGCTTTATCTGTC | |
| terminator | TTTGTATTGTTTCATCAATTCATGTAACGTTTGCTTTTCTTATGAATT | |
| (TerMas) | TTCAAATAAATTATC | |
| Agrobacterium | GAATTAACAGAGGTGGATGGACAGACCCGTTCTTACACCGGACTGGGC | 82 |
| tumefaciens | GCGGGATAGGATATTCAGATTGGGATGGGATTGAGCTTAAAGCCGGCG | |
| agropine | CTGACACCATGCTCAAGGTAGGCAATGTCCTCAGCGTCGAGCCCGGCA | |
| synthase | TCTATGTCGAGGGCATTGGTGGAGCGCGCTTCGGGGATACCGTGCTTG | |
| terminator | TAACTGAGAGCGGATATGAGGCCCTCACTCCGCTTGATCTTGGCAAAG | |
| (TerAgs) | ATATTTGACGCATTTATTAGTATGTGTTAATTTTCATTTGCAGTGCAG | |
| | TATTTTCTATTCGATCTTTATGTAATTCGTTACAATTAATAAATATTC | |
| | AAATCAGATTATTGACTGTCATTTGTATCAAATCGTGTTTAATGGATA | |
| | TTTTTATTATAATATTGATGAT | |
| Terminator | TGCGAGAAGCCTATGATCGCATGATATTTGCTTTCAATTCTGTTGTGC | 83 |
| OCS | ACGTTGTAAAAAACCTGAGCATGTGTAGCTCAGATCCTTACCGCCGGT | |
| | TTCGGTTCATTCTAATGAATATATCACCCGTTACTATCGTATTTTTAT | |
| | GAATAATATTCTCCGTTCAATTTACTGATTGTACCCTACTACTTATAT | |
| | GTACAATATTAAAATGAAAACAATATATTGTGCTGAATAGGTTTATAG | |
| | CGACATCTATGATAGAGCGCCACAATAACAAACAATTGCGTTTTATTA | |
| | TTACAAATCCAATTTTAAAAAAAGCGGCAGAACCGGTCAAACCTAAAA | |
| | GACTGATTACATAAATCTTATTCAAATTTCAAAAGTGCCCCAGGGGCT | |
| | AGTATCTACGACACACCGAGCGGCGAACTAATAACGCTCACTGAAGGG | |
| | AACTCCGGTTCCCCGCCGGCGCGCATGGGTGAGATTCCTTGAAGTTGA | |
| | GTATTGGCCGTCCGCTCTACCGAAAGTTACGGGCACCATTCAACCCGG | |
| | TCCAGCACGGCGGCCGGGTAACCGACTTGCTGCCCCGAGAATTATGCA | |
| | GCATTTTTTTGGTGTATGTGGGCCCCAAATGAAGTGCAGGTCAAACCT | |
| | TGACAGTGACGACAAATCGTTGGGCGGGTCCAGGGCGAATTTTGCGAC | |
| | AACATGTCGAGGCTCAGCAGGACCGCTACTAGAATT | |
| Terminator | GTCCTGCTTTAATGAGATATGCGAGAAGCCTATGATCGCATGATATTT | 178 |
| OCS (2) | GCTTTCAATTCTGTTGTGCACGTTGTAAAAAACCTGAGCATGTGTAGC | |
| | TCAGATCCTTACCGCCGGTTTCGGTTCATTCTAATGAATATATCACCC | |
| | GTTACTATCGTATTTTTATGAATAATATTCTCCGTTCAATTTACTGAT | |
| | TGTACCCTACTACTTATATGTACAATATTAAAATGAAAACAATATATT | |
| | GTGCTGAATAGGTTTATAGCGACATCTATGATAGAGCGCCACAATAAC | |
| | AAACAATTGCGTTTTATTATTACAAATCCAATTTTAAAAAAAGCGGCA | |
| | GAACCGGTCAAACCTAAAAGACTGATTACATAAATCTTATTCAAATTT | |
| | CAAAAGTGCCCCAGGGGCTAGTATCTACGACACACCGAGCGGCGAACT | |
| | AATAACGCTCACTGAAGGGAACTCCGGTTCCCCGCCGGCGCGCATGGG | |
| | TGAGATTCCTTGAAGTTGAGTATTGGCCGTCCGCTCTACCGAAAGTTA | |
| | CGGGCACCATTCAACCCGGTCCAGCACGGCGGCCGGGTAACCGACTTG | |
| | CTGCCCCGAGAATTATGCAGCATTTTTTTGGTGTATGTGGGCCCCAAA | |
| | TGAAGTGCAGGTCAAACCTTGACAGTGACGACAAATCGTTGGGCGGGT | |
| | CCAGGGCGAATTTTGCGACAACATGTCGAGGCTCAGCAGGAC | |
| 3′UTR + | CTCTAGCTAGAGTCGATCGACAAGCTCGAGTTTCTCCATAATAATGTG | 140 |
| 35S | TGAGTAGTTCCCAGATAAGGGAATTAGGGTTCCTATAGGGTTTCGCTC | |
| terminator | ATGTGTTGAGCATATAAGAAACCCTTAGTATGTATTTGTATTTGTAAA | |
| | ATACTTCTATCAATAAAATTTCTAATTCCTAAAACCAAAATCCAGTAC | |
| | TAAAATCCAGAT | |
| Mas | TTGGACTCCCATGTTGGCAAAGGCAACCAAACAAACAATGAATGATCC | 141 |
| terminator | GCTCCTGCATATGGGGCGGTTTGAGTATTTCAACTGCCATTTGGGCTG | |
| (2) | AATTGTAGACATGCTCCTGTCAGAAATTCCGTGATCTTACTCAATATT | |
| | CAGTAATCTCGGCCAATATCCTAAATGTGCGTGGCTTTATCTGTCTTT | |
| | GTATTGTTTCATCAATTCATGTAACGTTTGCTTTTCTTATGAATTTTC | |
| | AAATAAATTATC | |
| Ocs | GTCCTGCTTTAATGAGATATGCGAGAAGCCTATGATCGCATGATATTT | 142 |
| terminator | GCTTTCAATTCTGTTGTGCACGTTGTAAAAAACCTGAGCATGTGTAGC | |
| (3) | TCAGATCCTTACCGCCGGTTTCGGTTCATTCTAATGAATATATCACCC | |
| | GTTACTATCGTATTTTTATGAATAATATTCTCCGTTCAATTTACTGAT | |
| | TGTACCCTACTACTTATATGTACAATATTAAAATGAAAACAATATATT | |
| | GTGCTGAATAGGTTTATAGCGACATCTATGATAGAGCGCCACAATAAC | |
| | AAACAATTGCGTTTTATTATTACAAATCCAATTTTAAAAAAAGCGGCA | |
| | GAACCGGTCAAACCTAAAAGACTGATTACATAAATCTTATTCAAATTT | |
| | CAAAAGTGCCCCAGGGGCTAGTATCTACGACACACCGAGCGGCGAACT | |
| | AATAACGCTCACTGAAGGGAACTCCGGTTCCCCGCCGGCGCGCATGGG | |
| | TGAGATTCCTTGAAGTTGAGTATTGGCCGTCCGCTCTACCGAAAGTTA | |
| | CGGGCACCATTCAACCCGGTCCAGCACGGCGGCCGGGTAACCGACTTG | |
| | CTGCCCCGAGAATTATGCAGCATTTTTTTGGTGTATGTGGGCCCCAAA | |
| | TGAAGTGCAGGTCAAACCTTGACAGTGACGACAAATCGTTGGGCGGGT | |
| | CCAGGGCGAATTTTGCGACAACATGTCGAGGCTCAGCAGGAC | |
| 35S | CTCTAGCTAGAGTCGATCGACAAGCTCGAGTTTCTCCATAATAATGTG | 143 |
| terminator | TGAGTAGTTCCCAGATAAGGGAATTAGGGTTCCTATAGGGTTTCGCTC | |
| (2) | ATGTGTTGAGCATATAAGAAACCCTTAGTATGTATTTGTATTTGTAAA | |
| | ATACTTCTATCAATAAAATTTCTAATTCCTAAAACCAAAATCCAGTAC | |
| | TAAAATCCAGAT | |
| Nicotiana | GAAGTGACATCACAAAGTTGAAGGTAATAAAGCCAAATTAATTAAGAC | 144 |
| tabacum | ATTTTCATAATGATGTCAAGAATGCAAAGCAAATTGCATAACTGCCTT | |
| extensin 3 | TATGCAAAACATTAATATAATATAAATTATAAAGAACTGCGCTCTCTG | |
| terminator | CTTCTTATTTTCTTAGCTTCATTTATTAGTCACTAGCTGTTCAGAATT | |
| (NtExt3) | TTCAGTATCTTTTGATATTACTAAGAACCTAATCACACAATGTATATT | |
| | CTTATGCAGGAAAAGCAGAATGCTGAGCTAAAAGAAAGGCTTTTTCCA | |
| | TTTTCGAGAGACAATGAGAAAAGAAGAAGAAGAAGAAGAAGAAGAAGA | |
| | AGAAGAAGAAGAAAAGAGTAAATAATAAAGCCCCACAGGAGGCGAAGT | |
| | TCTTGTAGCTCCATGTTATCTAAGTTATTGATATTGTTTGCCCTATAT | |
| | TTTATTTCTGTCATTGTGTATGTTTTGTTCAGTTTCGATCTCCTTGCA | |
| | AAATGCAGAGATTATGAGATGAATAAACTAAGTTATATTATTATACGT | |
| | GTTAATATTCTCCTCCTCTCTCTAGCTAGCCTTTTGTTTTCTCTTTTT | |
| | CTTATTTGATTTTCTTTAAATCAATCCATTTTAGGAGAGGGCCAGGGA | |
| | GTGATCCAGCAAAACATGAAGATTAGAAGAAACTTCCCTCTTTTTTTC | |
| | CTGAAAACAATTTAACGTCGAGATTTATCTCTTTTTGTAATGGAATCA | |
| | TTTCTACAGTTATGAC | |
| Arabidopsis | ATATGAAGATGAAGATGAAATATTTGGTGTGTCAAATAAAAAGCTTGT | 145 |
| thaliana | GTGCTTAAGTTTGTGTTTTTTTCTTGGCTTGTTGTGTTATGAATTTGT | |
| Heat Shock | GGCTTTTTCTAATATTAAATGAATGTAAGATCTCATTATAATGAATAA | |
| Protein 18.2 | ACAAATGTTTCTATAATCCATTGTGAATGTTTTGTTGGATCTCTTCTG | |
| terminator | CAGCATATAACTACTGTATGTGCTATGGTATGGACTATGGAATATGAT | |
| (AtHSP18.2) | TAAAGATAAG | |
| Nos | GTCAAGCAGATCGTTCAAACATTTGGCAATAAAGTTTCTTAAGATTGA | 146 |
| terminator | ATCCTGTTGCCGGTCTTGCGATGATTATCATATAATTTCTGTTGAATT | |
| (2) | ACGTTAAGCATGTAATAATTAACATGTAATGCATGACGTTATTTATGA | |
| | GATGGGTTTTTATGATTAGAGTCCCGCAATTATACATTTAATACGCGA | |
| | TAGAAAACAAAATATAGCGCGCAAACTAGGATAAATTATCGCGCGCGG | |
| | TGTCATCTATGTTACTAGATCGA | |
| Solanum | ATATGTCAACAGTGAGAAACTGTTCGCATTTTCCGTTTTGCTTCTTTC | 179 |
| lycopersicum | TTTCTATTCAATGTATGTTGTTGGATTCCAGTTGAATTTATTATGAGA | |
| Rubisco | ACTAATAATAATAGTAATAATCATTTGTTTCTTTACTAATTTGCATTT | |
| small | TCACATATGATTTCTGGTGCATATCATAATTTTCATTCCACCAATATT | |
| subunit 3C | AATTTCCCCATTCAAGTTACTTATGAAATAGAAATCCTCTTCTCCGAC | |
| terminator | TACTTTATTTGTCCGAAAGTCTTGTGGCTGCTATATAA | |
| (Ter | | |
| S1RbcS3C) | | |
| Agrobacterium | GATCCCCCGTCGACAGCTAGCTATATCATCAATTTATGTATTACACAT | 180 |
| tumefaciens | AATATCGCACTCAGTCTTTCATCTACGGCAATGTACCAGCTGATATAA | |
| gene 7 | TCAGTTATTGAAATATTTCTGAATTTAAACTTGCATCAATAAATTTAT | |
| terminator | GTTTTTGCTTGGACTATAATACCTGACTTGTTATTTTATCAATAAATA | |
| (Ter Atug7) | TTTAAACTATATTTCTTTCAAGAT | |
| Epipremnum | GCTGTAAAGAAATTGATGGGCAGTGGGCTTTTGTTACTAGTTAGTAGG | 181 |
| aureum rrEa | AGAGGTTGCTTCAGTTTCGTCCGTACCTGTTCTTGACCTTCTGTTTCT | |
| H3Blast2 | GGAGTCTGTACTCCGTTTGTTGTAAAGTCTTGTCCTTTTTTTAAAACT | |
| terminator | TCTTTCTATCCACTGTTGAATGAGCCAGTAGATGCTGTCCTGTTACGC | |
| (Ter 7.1) | GTTTCTCTTCTCTTGCACATGCACAGTCTCCGTTTTGTAGGATGCTGA | |
| | ACGAAGCTCTCGGGTTTATGGAGGTCAATCCCTAAGTATTGTCGATTC | |
| | AAAAGGGTGATGTTTTTTTCCCCCAACAAAGCTCTTCAGTGAGTTCAA | |
| | CCAAGTGGGTGAGATGTGTATAGGTTACTGGACAATCTTGTTGGTTTG | |
| | GAGAGGAGAAAAAGTAGCTATATTGATCTGTGCCAGTGCTAGCACAGG | |
| | GAGAGTCTTATCTTTTTGGGTTAGTGTTACAGCTAGATGATTGAGATG | |
| | ATCATCTGCACTTGATTTGATCAGCTGGTTTTGTCTTTGTAAGATTAG | |
| | CCTGTCACTTGACGAAAAAAAGCGGTTTGTCTGTCCTCGGTTACGATT | |
| | CAGACTGGTTTGGATGACGTCCATATTAAGATCCTGTATTTACGTTTG | |
| | CTGCTCTCATTTTCTGCAAGCTTTCCGAGGATGTCCAAAAGCT | |
| Solanum | CTAGACTTGTCCATCTTCTGGATTGGCCAACTTAATTAATGTATGAAA | 182 |
| tuberosum | TAAAAGGATGCACACATAGTGACATGCTAATCACTATAATGTGGGCAT | |
| Proteinase | CAAAGTTGTGTGTTATGTGTAATTACTAGTTATCTGAATAAAAGAGAA | |
| inhibitor 2 | AGAGATCATCCATATTTCTTATCCTAAATGAATGTCACGTGTCTTTAT | |
| terminator | AATTCTTTGATGAACCAGATGCATTTCATTAACCAAATCCATATACAT | |
| (Ter StPinII) | ATAAATATTAATCATATATAATTAATATCAATTGGGTTAGCAAAACAA | |
| | ATCTAGTCTAGGTGTGTTTTGC | |
| Pisum | TCCAGGCCTCCCAGCTTTCGTCCGTATCATCGGTTTCGACAACGTTCG | 183 |
| sativum | TCAAGTTCAATGCATCAGTTTCATTGCCCACACACCAGAATCCTACTA | |
| Rubisco | AGTTTGAGTATTATGGCATTGGAAAAGCTGTTTTCTTCTATCATTTGT | |
| small | TCTGCTTGTAATTTACTGTGTTCTTTCAGTTTTTGTTTTCGGACATCA | |
| subunit 3A | AAATGCAAATGGATGGATAAGAGTTAATAAATGATATGGTCCTTTTGT | |
| terminator | TCATTCTCAAATTATTATTATCTGTTGTTTTTACTTTAATGGGTTGAA | |
| (Ter Pea3A) | TTTAAGTAAGAAAGGAACTAACAGTGTGATATTAAGGTGCAATGTTAG | |
| | ACATATAAAACAGTCTTTCACCTCTCTTTGGTTATGTCTTGAATTGGT | |
| | TTGTTTCTTCACTTATCTGTGTAATCAAGTTTACTATGAGTCTATGAT | |
| | CAAGTAATTATGCAATCAAGTTAAGTACAGTATAGGCTT | |
| Arabidopsis | TCTCTTCTGCAGCATATAACTACTGTATGTGCTATGGTATGGACTATG | 184 |
| thaliana | GAATATGATTAAAGATAAGATGGGCTCATAGAGTAAAACGAGGCGAGG | |
| Heat Shock | GACCTATAAACCTCCCTTCATCATGCTATTTCATGATCTATTTTATAA | |
| Protein | AATAAAGATGTAGAAAAAAGTAAGCGTAATAACCGCAAAACAAATGAT | |
| terminator | TTAAAACATGGCACATAATGAGGAGATTAAGTTCGGTTTACGTTTATT | |
| (Ter | TTAGTACTAATT | |
| AtHSP) | | |

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated polypeptides aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

### Enhancers

In some embodiments, a construct described herein includes an enhancer sequence. The term “enhancer” refers to a nucleotide sequence that can increase the level of transcription of a nucleic acid encoding a polypeptide of interest. Enhancer sequences (generally 50-1500 bp in length) generally increase the level of transcription by providing additional binding sites for transcription-associated polypeptides (e.g., transcription factors). Unlike promoter sequences, in some embodiments certain enhancer sequences can act at much larger distance away from the transcription start site (e.g., as compared to a promoter). In some embodiments, an enhancer sequence is found within an intronic sequence. In some embodiments, an enhancer is an intronic sequence. In some embodiments, enhancers may act to decrease transcript degradation and/or silencing. In some embodiments, an enhancer may be inserted into the 5′ UTR of a construct. In some embodiments, an enhancer may be incorporated into a coding region of a transgene. In some embodiments, an intron acting as an enhancer may be an intron from a DEM1 gene, a DEM2 gene, a TCH3 gene, and/or a TRP1 gene. In some embodiments, additional non-limiting examples of enhancers include a RSV enhancer, a CMV enhancer, and/or a SV40 enhancer.

In some embodiments, an enhancer sequence is at least 85%, at least 90%, at least 95%, at least 98% or at least 99% identical to a sequence included in Table 5. In some embodiments, an enhancer sequence is a characteristic portion of a sequence included in Table 5.

TABLE 5
Exemplary Enhancer Polynucleotide Sequences

| | | SEQ ID |
|---|---|---|
| Description | Sequence | NO: |
| Arabidopsis | GTAAGCAGAACTCTAGTTGCAGTGTATATTCTTGCTGAGAAAGTGACA | 84 |
| thaliana | TTCTTGAAATTTTCATGTTTTGCTCATAGCATAAGTGCATATAATATT | |
| DEMI | GAAGTCTTAAGAATTTTTGTGGAAATTGAATTATAGTGTTCCTCAGTT | |
| intronic | GCCTTGTGTTTCAACCTTGATTTTTGATAGAGGAACTTTTACTACTGT | |
| nucleotide | TGAATCATTCATCAATTGAAATAACTTTTTACTAATAGTTGATTCCTG | |
| sequence. | ACTCTTTTTGTCTATCTTTTCTTGTTGAAAATGTCGATATATAG | |

### Flanking Untranslated Regions, 5′ UTRs and 3′ UTRs

In some embodiments, any of the construct described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, or methods as described herein to enhance or otherwise modulate the expression of a polypeptide.

Natural 5′ UTRs include a sequence that plays a role in translation initiation. In some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In some embodiments, 5′ UTRs have also been known to form secondary structures that are involved in elongation factor binding.

In some embodiments, 5′ UTR is one listed herein as included in Table 6. In some embodiments, a 5′ UTR sequence is at least 85%, at least 90%, at least 95%, at least 98% or at least 99% identical to a sequence included in Table 6. In some embodiments, a 5′ UTR sequence is a characteristic portion to a sequence included in Table 6.

TABLE 6
Exemplary UTR Polynucleotide Sequences

| | | SEQ ID |
|---|---|---|
| Description | Sequence | NO: |
| Exemplary | GTATTTTTACAACAATTACCAACAACAACAAACAACAAACAACATTAC | 85 |
| Tobacco | AATTACTATTTACAATTAC | |
| Mosaic | | |
| Virus | | |
| (TMV) 5′- | | |
| leader | | |
| sequence | | |
| (Omega). | | |
| Exemplary | TACATCACAATCACACAAAACTAACAAAAGATCAAAAGCAAGTTCTTC | 86 |
| Arabidopsis | ACTGTTGATA | |
| thaliana | | |
| Alcohol | | |
| Dehydrogen | | |
| ase 5′ UTR. | | |
| Exemplary | GTCTATTTCTCAGTATTCAGAAACAACAAAAGTTCTTCTCTACATAAA | 87 |
| Nicotiana | ATTTTCCTATTTTAGTGATCAGTGAAGGAAATCAAGAAAAATAA | |
| tabacum | | |
| Alcohol | | |
| Dehydrogen | | |
| ase 5′ UTR. | | |
| Exemplary | GAATTCCAAGCAACGAACTGCGAGTGATTCAAGAAAAAAGAAAACCTG | 88 |
| Oryza sativa | AGCTTTCGATCTCTACGGAGTGGTTTCTTGTTCTTTGAAAAAGAGGGG | |
| Alcohol | GATTA | |
| Dehydrogen | | |
| se 5′ UTR. | | |

### Internal Ribosome Entry Sites (IRES), Secretion Signals, and Cleavage Signals

In some embodiments, a construct described herein encoding a polypeptide can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8 (3): 1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15 (13): 1593-612, 2001, each of which is incorporated in its entirety herein by reference.

In some embodiments, a construct provided herein can include secretion signals, cleavage sites, and/or linker sequences. In some embodiments, these sites are functional in a translated polypeptide, and result in post-translational modifications and/or processing events. In some embodiments, constructs as described herein are translated into a relatively long precursor polypeptide, such a precursor polypeptide may then undergo post translational modifications and/or processing, which may involve endogenous cellular enzymatic actions. Such a processing step may produce multiple peptides, the biological function of such peptides may be accomplished either solely by one peptide, or by the function of multiple peptides acting in concert.

In some embodiments, constructs provided herein include a signal peptide. In some embodiments, a signal peptide may be a signal sequence, targeting signal, localization signal, localization sequence, transit peptide, leader sequence or leader peptide. In some embodiments, such a sequence is generally short (e.g., approximately 15-60 amino acids in length). In some embodiments, such a signal peptide is present at the N-terminus of a peptide of interest. In some embodiments, more than one signal peptide may exist in a translational product. In some embodiments, an exemplary signal peptide comprises a localization signal. One skilled in the art will recognize that alternative localization signal sequences exist, and may be incorporated into constructs as described herein.

In some embodiments, an IRES, secretion signal, and/or cleavage signal is one listed herein as included in Table 8 or encoded by a sequence as included in Table 7. In some embodiments, an IRES, secretion signal, and/or cleavage signal is at least 85%, at least 90%, at least 95%, at least 98% or at least 99% identical to a sequence included in in Table 8 or encoded by a sequence as included in Table 7. In some embodiments, an IRES, secretion signal, and/or cleavage signal is a characteristic portion to a sequence included in in Table 8 or encoded by a sequence as included in Table 7.

TABLE 7
Exemplary IRES, Secretion Signal, and/or Cleavage Signal
Polynucleotide and Polypeptide Sequences Sequences

| | | SEQ ID |
|---|---|---|
| Description | Sequence | NO: |
| Exemplary | GGCTCTGGCGAAGGCAGAGGCAGCCTGCTTACATGTGGCGACGTGGAA | 89 |
| Cleavage | GAGAACCCCGGACCT | |
| signal | | |
| nucleotide | | |
| sequence | | |
| Exemplary | GCCCCGGTGAAGCAGACCCTGAACTTCGACCTGCTGAAGCTGGCGGGC | 90 |
| Cleavage | GACGTGGAGAGCAACCCGGGCCCC | |
| signal | | |
| nucleotide | | |
| sequence | | |

TABLE 8
Exemplary IRES, Secretion Signal, and/or Cleavage Signal
Polypeptide Sequences

| | | SEQ ID |
|---|---|---|
| Description | Sequence | NO: |
| Exemplary | ASSMLSSAAVVTSPAQATMVAPFTGLKSSASFPVTRKANNDITSITSN | 94 |
| Chloroplast | GGRVSC | |
| localization | | |
| signal | | |
| amino acid | | |
| sequence | | |
| Exemplary | MAMAVFRREGRRLLPSIAARPIAAIRSPLSSDQEEGLLGVRSISTQVV | 95 |
| Mitochondria | RNR | |
| localization | | |
| signal | | |
| amino acid | | |
| sequence | | |
| Exemplary | MEKAIERQRVLLEHLRPSSSSSHNYEASLSASACLAGDSAAYQRTSLY | 96 |
| Peroxisome | G | |
| localization | | |
| signal | | |
| amino acid | | |
| sequence | | |
| Exemplary | GSGEGRGSLLTCGDVEENPGP | 97 |
| Cleavage | | |
| signal | | |
| amino acid | | |
| sequence | | |
| Exemplary | APVKQTLNFDLLKLAGDVESNPGP | 98 |
| Cleavage | | |
| signal | | |
| amino acid | | |
| sequence | | |

### Splice Sites and Introns

In some embodiments, constructs provided herein can include splice donor and/or splice acceptor sequences. In some embodiments, such a splice donor and/or splice acceptor sequence may be functional during RNA processing occurring during and/or following transcription. In some embodiments, splice sites are involved in trans-splicing. In some embodiments, splices sites are involved in cis-splicing.

### Cloning Site

In some embodiments, constructs of the present disclosure may include one or more cloning sites. In some such embodiments, cloning sites may not be fully removed prior to administration to a subject (e.g., a plant cell). In some embodiments, cloning sites may have functional roles, e.g., including as linker sequences, cleavage sequence, or as portions of a Kozak site. In some embodiments, cloning sites may vary significantly in primary sequence while retaining their desired function. In some embodiments, constructs may contain any appropriate combination of cloning sites.

### Additional Sequences

In some embodiments, a construct of the present disclosure comprises an additional sequence included in Table 9. In some embodiments, an insulator sequence is at least 85%, at least 90%, at least 95%, at least 98% or at least 99% identical to a sequence included in Table 9. In some embodiments, an insulator sequence has a sequence included in Table 9.

TABLE 9
Additional Polynucleotide Sequence

| | | SEQ ID |
|---|---|---|
| Description | Sequence | NO: |
| Insulator | ATCGAAGGTTCGCCTTTCTGTTGTAATATTGCTCAATCCTCTACTTTT | 100 |
| Sequence | GTGCCGCCATCTGTATCAAAGTTGCTTCCCTTATCTCCTTTGTCAAAT | |
| TM2MARS | CTAAATTTGTTAAAAGTTCTTTCTCATTTGTTACTTCATTTGTATGTA | |
| | CGTATCTTGTTCTTGGCTCTCCTATTTCCACCGAAATTAAGGCCTCAG | |
| | TACCATAAATAAGTGAGAATGGTGTTTCTCTTGTGCTTATCTTCGTCG | |
| | TTGTCCTGTAATCCCATACTAGCCCAAGTAGTACTTCAGGGAACCTGC | |
| | CTTTTGATGATTCTAACCGTTTCTTTATATTGTTGACAATAATTGTAT | |
| | TTGTCGATTATGGTTTCCCATTAGCTATGGGATGGCAGTGCGCCGAAA | |
| | TAATTTGTTTAATTTCCCAGATTTTGAGAAAATCGGTAACTTTTGAAC | |
| | CTACGAATTGTGGCCCATTATCGCGTACTATTTTTCTTGGGACACAAA | |
| | ATCGACATATTATGTTCCTCCAAATAATATCAACAACCTCTTTCTCTC | |
| | GATCTATTTAAAGGCACATGCTTCTACCCACATAGAAAAATAATCAGT | |
| | TAAGATTAATAAAAGTCGTTCCTTTCCTGGTGCTTTAGGTAGAGGACC | |
| | TACAATACATAACCCACTTCATAAACGGCCACGGTGCTATGATTGAAT | |
| | GAAGAAACACTGCTGGTTGATGCATATTGTTTGCACCCCGTTGACATT | |
| | TATCACACCTTCTAATAAAAATTTTCGCTTTGTGTTCTATTTTTTGCA | |
| | AATAATACCGCCCCTTTTAGCGTCTTTAAAATGACCTTCCTCCAACGT | |
| | GATTGCAACAATGTTCTTTATGTACTTCTTGCATCACATATTCAGTTT | |
| | GTGATAGACCAAGGCACCGTGCTAAGGGTCCCATGAACATTTCGATAC | |
| | AAATTTTCTTCTATTAAGCAATATTGAGCAGCTTTCACTCGCAATAAT | |
| | TGGGATTTTTAATCGACGCTTTACTGAGACCGAGGATGCACAT | |
| Tm2 MAR1 | GGAGAGGAAGAAGTTCCCAATCTTGAAATATCGAAGGTTCGCCTTTCT | 151 |
| | GTTGTAATATTGCTCAATCCTCTACTTTTGTGCCGCCATCTGTATCAA | |
| | AGTTGCTTCCCTTATCTCCTTTGTCAAATCTAAATTTGTTAAAAGTTC | |
| | TTTCTCATTTGTTACTTCATTTGTATGTACGTATCTTGTTCTTGGCTC | |
| | TCCTATTTCCACCGAAATTAAGGCCTCAGTACCATAAATAAGTGAGAA | |
| | TGGTGTTTCTCTTGTGCTTATCTTCGTCGTTGTCCTGTAATCCCATAC | |
| | TAGCCCAAGTAGTACTTCAGGGAACCTGCCTTTTGATGATTCTAACCG | |
| | TTTCTTTATATTGTTGACAATAATTGTATTTGTCGATTATGGTTTCCC | |
| | ATTAGCTATGGGATGGCAGTGCGCCGAAATAATTTGTTTAATTTCCCA | |
| | GATTTTGAGAAAATCGGTAACTTTTGAACCTACGAATTGTGGCCCATT | |
| | ATCGCGTACTATTTTTCTTGGGACACAAAATCGACATATTATGTTCCT | |
| | CCAAATAATATCAACAACCTCTTTCTCTCGATCTATTTAAAGGCACAT | |
| | GCTTCTACCCACATAGAAAAATAATCAGTTAAGATTAATAAAAGTCGT | |
| | TCCTTTCCTGGTGCTTTAGGTAGAGGACCTACAATACATAACCCACTT | |
| | CATAAACGGCCACGGTGCTATGATTGAATGAAGAAACACTGCTGGTTG | |
| | ATGCATATTGTTTGCACCCCGTTGACATTTATCACACCTTCTAATAAA | |
| | AATTTTCGCTTTGTGTTCTATTTTTTGCAAATAATACCGCCCCTTTTA | |
| | GCGTCTTTAAAATGACCTTCCTCCAACGTGATTGCAACAATGTTCTTT | |
| | ATGTACTTCTTGCATCACATATTCAGTTTGTGATAGACCAAGGCACCG | |
| | TGCTAAGGGTCCCATGAACATTTCGATACAAATTTTCTTCTATTAAGC | |
| | AATATTGAGCAGCTTTCACTCGCAATAATTGGGATTTTTAATCGACGC | |
| | TTTAC | |
| Tm2 MAR2 | AGGAAGAAGTTCCCAATCTTGAAATATCGAAGGTTCGCCTTTCTGTTG | 152 |
| | TAATATTGCTCAATCCTCTACTTTTGTGCCGCCATCTGTATCAAAGTT | |
| | GCTTCCCTTATCTCCTTTGTCAAATCTAAATTTGTTAAAAGTTCTTTC | |
| | TCATTTGTTACTTCATTTGTATGTACGTATCTTGTTCTTGGCTCTCCT | |
| | ATTTCCACCGAAATTAAGGCCTCAGTACCATAAATAAGTGAGAATGGT | |
| | GTTTCTCTTGTGCTTATCTTCGTCGTTGTCCTGTAATCCCATACTAGC | |
| | CCAAGTAGTACTTCAGGGAACCTGCCTTTTGATGATTCTAACCGTTTC | |
| | TTTATATTGTTGACAATAATTGTATTTGTCGATTATGGTTTCCCATTA | |
| | GCTATGGGATGGCAGTGCGCCGAAATAATTTGTTTAATTTCCCAGATT | |
| | TTGAGAAAATCGGTAACTTTTGAACCTACGAATTGTGGCCCATTATCG | |
| | CGTACTATTTTTCTTGGGACACAAAATCGACATATTATGTTCCTCCAA | |
| | ATAATATCAACAACCTCTTTCTCTCGATCTATTTAAAGGCACATGCTT | |
| | CTACCCACATAGAAAAATAATCAGTTAAGATTAATAAAAGTCGTTCCT | |
| | TTCCTGGTGCTTTAGGTAGAGGACCTACAATACATAACCCACTTCATA | |
| | AACGGCCACGGTGCTATGATTGAATGAAGAAACACTGCTGGTTGATGC | |
| | ATATTGTTTGCACCCCGTTGACATTTATCACACCTTCTAATAAAAATT | |
| | TTCGCTTTGTGTTCTATTTTTTGCAAATAATACCGCCCCTTTTAGCGT | |
| | CTTTAAAATGACCTTCCTCCAACGTGATTGCAACAATGTTCTTTATGT | |
| | ACTTCTTGCATCACATATTCAGTTTGTGATAGACCAAGGCACCGTGCT | |
| | AAGGGTCCCATGAACATTTCGATACAAATTTTCTTCTATTAAGCAATA | |
| | TTGAGCAGCTTTCACTCGCAATAATTGGGATTTTTAATCGA | |
| Pea MAR | CCATGCCTCACATGTTAATGTACTACCAATGGAGGGCTGTACACATTT | 153 |
| | ATGATTACGAAATTTTTTAATATATTTTATAGATTTCTTATGCATCAT | |
| | ACAAAAATACATAATTATTCGTAACATTTTGGAGATACATATTCAGAT | |
| | GCATCAAATTCTAATTAAACGTTAAAATATTTTGCAGACGTATCTTCG | |
| | TAACAATTTAAAACCTATACTATACATCACATTCGAAGGTCATTTTAT | |
| | AATTTAAAATATTATGGAGATGCATCTTCGTTTATGTTTGCTCAGATG | |
| | AAGATTTAAACCTTACAAACAATATGTAAAAAATGACGTACATAAATT | |
| | CAGATAGTCCAAAAGTGTCATATATAAATAAAGATCAATAAGTGTCAA | |
| | AAAAAGTCAAGAACAACGATAGAGTAGCATAATGTCAAAATAAAATAA | |
| | AATCCATGACACTACTACTATTATATATTAATGCACTAATGTGTATGT | |
| | CTAACTACATCGTCTGTGCCTCCTCTGTCAGTTATGTCTCGTAAGCCA | |
| | TCAATCCCCCGTCCTCCGGCGTTGTCTCCGGTATATCAATGTCCCATG | |
| | TGCCTACGTCATGATGGCATCTAGGACATGTCTCACATCAGACAATTA | |
| | GGAAAGATACATTTGCCAATGTATATCTGCGCAATCTCCACAATGCAA | |
| | CGACATATAGGCAAGACATCCTCAACATAATTTAGTTGTGCATGCTTC | |
| | TCCT | |
| Minimal | AACCAACTCGGTCCATTTGCACCCCTAATCATAATAGCTTTAATATTT | 154 |
| RB7 MAR | CAAGATATTATTAAGTTAACGTTGTCAATATCCTGGAAATTTTGCAAA | |
| | ATGAATCAAGCCTATATGGCTGTAATATGAATTTAAAAGCAGCTCGAT | |
| | GTGGTGGTAATATGTAATTTACTTGATTCTAAAAAAATATCCCAAGTA | |
| | TTAATAATTTCTGCTAGGAAGAAGGTTAGCTACGATTTACAGCAAAGC | |
| | CAGAATACAAAGAACCATAAAGTGATTGAAGCTCGAAATATACGAAGG | |
| | AACAAATATTTTTAAAAAAATACGCAATGACTTGGAACAAAAGAAAGT | |
| | GATATATTTTTTGTTCTTAAACAAGCATCCCCTCTAAAGAATGGCAGT | |
| | TTTCCTTTGCATGTAACTATTATGCTCCCTTCGTTACAAAAATTTTGG | |
| | ACTACTATTGGGAACTTCTTCTGAAAATAGT | |

### Reporter Sequences or Elements

In some embodiments, constructs provided herein can include a reporter polynucleotide sequence for a reporter gene that may encode a polypeptide. In some embodiments, a reporter gene imparts a distinct phenotype to a cell expressing a reporter polypeptide, thus allowing cells transformed with a construct described herein to be distinguished from cells that do not comprise said construct. Such genes may encode, for example, a selectable and/or screenable reporter. In some embodiments, nucleic acid constructs described herein comprise a polynucleotide sequence encoding a reporter that allows selecting and/or screening of transformed cells.

In some embodiments, a transformed cell is grown in culture medium under conditions that select for cells that either have (positive selection) or do not have (negative selection) the reporter. In some embodiments, a combination of positive and negative selection is used. In some so-called positive selection schemes, most cells in a population are unable reproduce, e.g., because they lack the ability to use a nutrient (such as, for example, a carbon source) present in the selection medium. In some of these schemes, the selectable reporter confers an ability to use a limiting nutrient. Thus, in some embodiments, cells that have the selectable reporter gain an advantage over other cells in the population and therefore can be selected for. In some so-called negative screening/selection schemes, most cells in a population are unable to divide because of the effects of a toxic agent (such as, for example, an antibiotic present in the selection medium). In these schemes, the selectable reporter confers an ability to overcome the toxicity (for example, by blocking uptake or by chemically modifying the toxic agent). Thus, in some embodiments, cells that have the selectable reporter gain an advantage over other cells in the population and therefore can be selected for. In some embodiments, a transformed cell undergoing selection is a prokaryotic cell, e.g., such as E. coli or an Agrobacterium etc., In some embodiments, a transformed cell undergoing selection is a eukaryotic cell, such as a plant cell, yeast (for example, S. cerevisiae), mammalian cell, or insect cell. In some embodiments, a characteristic phenotype allows the identification of cells of interest, groups of cells, tissues, organs, plant parts or whole plants containing a construct of interest.

In some embodiments, constructs described herein may include one or more nucleotide sequences encoding an appropriate selection and/or screening marker. In some embodiments, an appropriate selection marker may be encoded by nptII and/or kana and provide resistance to kanamycin. In some embodiments, an appropriate selection marker may be encoded by hpt and provide resistance to hyromycin. In some embodiments, an appropriate selection marker may be encoded by bar and provide resistance to phosphinothricin. In some embodiments, an appropriate selection marker may be encoded by gox and provide resistance to glyphosate. In some embodiments, an appropriate selection marker system includes neomycin phosphotransferase. In some embodiments, an appropriate selection marker system includes hygromycin phosphotransferase. In some embodiments, an appropriate selection marker system includes phosphoinothricin acetyltransferase. In some embodiments, an appropriate selection marker system includes glyphosate oxidoreductase.

Many examples of suitable reporter genes are known in the art and can be used in screening and/or selection schemes during methods described herein and/or during creation of compositions described herein. Reagents such as appropriate components of selection media are also known in the art. Examples of such reporter genes include, but are not limited to, phosphomannose isomerase, phosphinothricin, neomycin phosphotransferase, hygromycin phosphotransferase, enolpyruvoyl-shikimate-3-phosphate synthetase, etc.

For example, phosphomannose isomerase (PMI) catalyses the interconversion of mannose 6-phosphate and fructose 6-phosphate in prokaryotic and eukaryotic cells. After uptake, mannose is phosphorylated by endogenous hexokinases to mannose-6-phosphate. Accumulation of mannose-6-phosphate leads to a block in glycolysis by inhibition of phosphoglucose-isomerase, resulting in severe growth inhibition. Phosphomannose-isomerase is encoded by the manA gene from Escherichia coli and catalyzes the conversion of mannose-6-phosphate to fructose-6-phosphate, an intermediate of glycolysis. On media containing mannose, manA expression in transformed plant cells relieves the growth inhibiting effect of mannose-6-phosphate accumulation and permits utilization of mannose as a source of carbon and energy, allowing transformed cells to grow.

In some embodiments, reporter genes encode polypeptides that generate a detectable phenotype. Non-limiting examples of suitable reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. Alternatively or additionally, a reporter gene can provide some other visibly reactive response (e.g., may cause a distinctive appearance such as color or growth pattern relative to organisms or cells not expressing the selectable reporter gene in the presence of some substance, either as applied directly to the organism or cells or as present in the tissue or cell growth media). For example, it is known in the art that transcriptional activators of anthocyanin biosynthesis, operably linked to a suitable promoter in a vector, have widespread utility as non-phytotoxic markers for plant cell transformation.

In some embodiments, a reporter gene may be but is not limited to eGFP, mCherry, mRubyd2, RRvT, mTFP1, RFP611, dTFP0.2, meffCFP, folding reporter GFP, ccalOFP1, tdKatushka2, vsfGFP-0, eYGFPuv, or any combination thereof.

In some embodiments, when reporter genes are associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a vector carrying the LacZ gene in a plant cell is detected by assays for beta-galactosidase activity. When the reporter is a fluorescent polypeptide (e.g., green fluorescent protein) or luciferase, the presence of a vector carrying the fluorescent polypeptide or luciferase in a plant cell may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the vectors described herein.

In some embodiments, a reporter sequence is a FLAG tag (e.g., a 3×FLAG tag), and the presence of a vector carrying the FLAG tag in a plant cell is detected by polypeptide binding or detection assays (e.g., Western blots, immunohistochemistry, radioimmunoassay (RIA), mass spectrometry).

In some embodiments, a reporter polypeptide sequence described herein is as included in Table 11. In some embodiments, a polynucleotide sequence encoding a reporter polypeptide sequence is as included in Table 10. In some embodiments, a reporter polypeptide sequence described herein is at least 85%, at least 90%, at least 95%, at least 98% or at least 99% identical to a sequence included in Table 11. In some embodiments, a polynucleotide sequence encoding a reporter polypeptide sequence is at least 85%, at least 90%, at least 95%, at least 98% or at least 99% identical to a sequence included in Table 10. In some embodiments, an IRES, secretion signal, and/or cleavage signals is a characteristic portion to a sequence included in Table 11 or encoded by a sequence as included in Table 10.

TABLE 10
Exemplary Reporter Polynucleotide Sequences

| | | SEQ ID |
|---|---|---|
| Description | Sequence | NO: |
| Exemplary | ATGGTGAGCAAGGGCGAGGAGCTGTTCACCGGGGTGGTGCCCATCCTG | 101 |
| eGFP | GTCGAGCTGGACGGCGACGTAAACGGCCACAAGTTCAGCGTGTCCGGC | |
| reporter | GAGGGCGAGGGCGATGCCACCTACGGCAAGCTGACCCTGAAGTTCATC | |
| nucleotide | TGCACCACCGGCAAGCTGCCCGTGCCCTGGCCCACCCTCGTGACCACC | |
| sequence | CTGACCTACGGCGTGCAGTGCTTCAGCCGCTACCCCGACCACATGAAG | |
| | CAGCACGACTTCTTCAAGTCCGCCATGCCCGAAGGCTACGTCCAGGAG | |
| | CGCACCATCTTCTTCAAGGACGACGGCAACTACAAGACCCGCGCCGAG | |
| | GTGAAGTTCGAGGGCGACACCCTGGTGAACCGCATCGAGCTGAAGGGC | |
| | ATCGACTTCAAGGAGGACGGCAACATCCTGGGGCACAAGCTGGAGTAC | |
| | AACTACAACAGCCACAACGTCTATATCATGGCCGACAAGCAGAAGAAC | |
| | GGCATCAAGGTGAACTTCAAGATCCGCCACAACATCGAGGACGGCAGC | |
| | GTGCAGCTCGCCGACCACTACCAGCAGAACACCCCCATCGGCGACGGC | |
| | CCCGTGCTGCTGCCCGACAACCACTACCTGAGCACCCAGTCCGCCCTG | |
| | AGCAAAGACCCCAACGAGAAGCGCGATCACATGGTCCTGCTGGAGTTC | |
| | GTGACCGCCGCCGGGATCACTCTCGGCATGGACGAGCTGTACAAG | |
| Exemplary | ATGGTGAGCAAGGGCGAGGAGGATAACATGGCCATCATCAAGGAGTTC | 102 |
| mCherry | ATGCGCTTCAAGGTGCACATGGAGGGCTCCGTGAACGGCCACGAGTTC | |
| reporter | GAGATCGAGGGCGAGGGCGAGGGCCGCCCCTACGAGGGCACCCAGACC | |
| nucleotide | GCCAAGCTGAAGGTGACCAAGGGTGGCCCCCTGCCCTTCGCCTGGGAC | |
| sequence | ATCCTGTCCCCTCAGTTCATGTACGGCTCCAAGGCCTACGTGAAGCAC | |
| | CCCGCCGACATCCCCGACTACTTGAAGCTGTCCTTCCCCGAGGGCTTC | |
| | AAGTGGGAGCGCGTGATGAACTTCGAGGACGGCGGCGTGGTGACCGTG | |
| | ACCCAGGACTCCTCCCTGCAGGACGGCGAGTTCATCTACAAGGTGAAG | |
| | CTGCGCGGCACCAACTTCCCCTCCGACGGCCCCGTAATGCAGAAGAAA | |
| | ACCATGGGCTGGGAGGCCTCCTCCGAGCGGATGTACCCCGAGGACGGC | |
| | GCCCTGAAGGGCGAGATCAAGCAGAGGCTGAAGCTGAAGGACGGCGGC | |
| | CACTACGACGCTGAGGTCAAGACCACCTACAAGGCCAAGAAGCCCGTG | |
| | CAGCTGCCCGGCGCCTACAACGTCAACATCAAGTTGGACATCACCTCC | |
| | CACAACGAGGACTACACCATCGTGGAACAGTACGAACGCGCCGAGGGC | |
| | CGCCACTCCACCGGCGGCATGGACGAGCTGTACAAGTAA | |
| Exemplary | ATGGTGTCAAAAGGTGAGGAGCTAATCAAAGAGAACATGCGAATGAAA | 103 |
| mRuby | GTGGTCATGGAAGGGAGCGTAAACGGCCACCAGTTCAAATGCACAGGC | |
| reporter | GAGGGCGAGGGCAACCCATACATGGGTACGCAGACCATGAGGATAAAA | |
| nucleotide | GTAATCGAGGGTGGTCCGTTGCCATTCGCCTTCGACATCCTGGCAACC | |
| sequence | TCGTTCATGTACGGGAGTCGAACATTCATCAAATACCCAAAAGGTATA | |
| | CCGGACTTCTTCAAACAGAGTTTCCCGGAAGGTTTCACCTGGGAGCGG | |
| | GTCACAAGGTACGAGGACGGTGGTGTCGTGACAGTAATGCAGGACACA | |
| | TCCTTAGAGGACGGTTGCCTGGTCTACCACGTCCAGGTGCGTGGCGTC | |
| | AACTTCCCCTCAAACGGCCCAGTAATGCAGAAGAAAACCAAAGGTTGG | |
| | GAGCCGAACACAGAGATGATGTACCCGGCGGACGGTGGCCTGCGTGGT | |
| | TACACACACATGGCATTAAAAGTGGACGGTGGTGGTCACCTCTCGTGC | |
| | TCGTTCGTCACAACCTACCGAAGCAAGAAAACGGTCGGGAACATCAAA | |
| | ATGCCGGGTATACACGCAGTCGACCACCGTCTCGAGCGTTTAGAGGAG | |
| | AGCGACAACGAGATGTTCGTCGTGCAGCGAGAGCACGCAGTGGCCAAA | |
| | TTCGCGGGTCTAGGCGGCGGGATGGACGAGTTATACAAATGA | |
| Exemplary | ATGGTATCAAAAGGGGAAGAGGTGATCAAAGAGTTCATGCGTTTCAAA | 104 |
| RRvT | GTACGAATGGAAGGTTCCATGAACGGGCACGAGTTCGAGATAGAGGGT | |
| reporter | GAGGGTGAGGGTAGGCCATACGAGGGCACACAGACGGCCAAACTGAAA | |
| nucleotide | GTAACCAAAGGTGGCCCACTCCCATTCGCGTGGGACATCTTGAGTCCA | |
| sequence | CAGTTCATGTACGGTAGCAAAGCCTACGTCAAACACCCGGCCGACATA | |
| | CCAGACTACAAGAAACTAAGTTTCCCAGAGGGGTTCAAATGGGAGCGA | |
| | GTAATGAACTTCGAGGACGGCGGCCTGGTCACGGTGACCCAGGACTCG | |
| | AGTTTACAGGACGGTACCTTGATATACAACGTCAAAATGCGGGGTACA | |
| | AACTTTCCCCCAGACGGCCCCGTAATGCAGAAGAAAACAATGGGTTGG | |
| | GAAGCAAGCACAGAGCGTTTGTACCCAAGGGACGGTGTGCTAAAAGGT | |
| | GAGATCCACCAGGCACTAAAATTAAAAGACGGCGGTCACTACCTAGTC | |
| | GAGTTCAAAACCATATACATGGCGAAGAAACCCGTGCAGCTCCCAGGT | |
| | TACTACTACGTAGACACCAAATTAGACATCACGTCGCACAACGAGGAC | |
| | TACACGATCGTCGAGCAGTACGAGCGTAGCGAGGGTCGACACCACCTC | |
| | TTCCTATACGGTATGGACGAGCTCTACAAA | |
| Exemplary | ATGGTCAGTAAAGGTGAGGAGACGACGATGGGTGTCATAAAACCAGAC | 105 |
| mTFP1 | ATGAAAATAAAACTGAAAATGGAAGGTAACGTCAACGGCCACGCATTC | |
| reporter | GTAATCGAGGGTGAGGGTGAGGGGAAACCATACGACGGGACGAACACC | |
| nucleotide | ATAAACCTGGAAGTGAAAGAGGGTGCCCCACTACCATTCTCATACGAC | |
| sequence | ATCCTGACAACCGCGTTCGCCTACGGTAACAGGGCATTCACCAAATAC | |
| | CCCGACGACATCCCAAACTACTTCAAACAGTCATTCCCAGAGGGTTAC | |
| | AGTTGGGAGAGGACAATGACATTCGAGGACAAAGGGATCGTGAAAGTG | |
| | AAAAGCGACATCAGCATGGAAGAGGACTCCTTCATCTACGAGATCCAC | |
| | TTGAAAGGTGAGAACTTCCCACCCAACGGTCCCGTAATGCAGAAGAAA | |
| | ACAACCGGTTGGGACGCATCAACCGAGCGGATGTACGTAAGGGACGGC | |
| | GTCTTAAAAGGTGACGTGAAACACAAACTGCTGTTGGAAGGTGGTGGG | |
| | CACCACAGGGTCGACTTCAAAACCATATACCGAGCAAAGAAAGCCGTG | |
| | AAATTGCCAGACTACCACTTCGTCGACCACCGGATAGAGATACTAAAC | |
| | CACGACAAAGACTACAACAAAGTAACCGTGTACGAGAGTGCCGTAGCG | |
| | CGAAACTCCACAGACGGCATGGACGAGCTGTACAAATGA | |
| Exemplary | ATGAACTCATTAATCAAAGAGAACATGCGTATGATGGTGGTCATGGAA | 106 |
| RFP611 | GGCTCGGTCAACGGTTACCAGTTCAAATGCACAGGTGAGGGTGACGGT | |
| reporter | AACCCATACATGGGTACCCAGACAATGCGTATCAAAGTGGTAGAGGGC | |
| nucleotide | GGTCCATTGCCCTTCGCGTTCGACGTACTGGCAACCAGTTTCATGTAC | |
| sequence | GGTTCAAAGACGTTCATCAAACACACCAAAGGTATACCCGACTTCTTC | |
| | AAACAGTCATTCCCAGAGGGTTTCACATGGGAGCGGGTGACGAGGTAC | |
| | GAGGACGGTGGTGTCATCACCGTGATGCAGGACACATCGCTCGAGGAC | |
| | GGCTGCTTGGTGTACCACGCCAAAGTGACGGGCGTCAACTTCCCCAGT | |
| | AACGGTGCAGTCATGCAGAAGAAAACGAAAGGGTGGGAGCCAAACACG | |
| | GAGATGTTATACCCCGCCGACGGCGGTCTGCGAGGTTACAGTCAGATG | |
| | GCCCTGAACGTGGACGGGGGGGGTTACTTGTCGTGCTCCTTCGAGACA | |
| | ACGTACAGGAGTAAGAAAACGGTAGAGAACTTCAAAATGCCAGGCTTC | |
| | CACTTCGTCGACCACCGTTTGGAGCGTCTCGAGGAGAGTGACAAAGAG | |
| | ATGTTCGTGGTCCAGCACGAGCACGCCGTGGCAAAATTCTGCGATCTC | |
| | CCATCAAAACTCGGTAGGCTGTAG | |
| Exemplary | ATGGTGTCGAAAGGTGAGGAGACGACTATGGGCGTGATCAAACCAGAC | 107 |
| dTFP0.2 | ATGAAAATCAAACTGAAAATGGAAGGTAACGTCAACGGTCACGCATTC | |
| reporter | GTAATCGAGGGTGAAGGGGAAGGCAAACCATACGACGGTACAAACACA | |
| nucleotide | GTCAACTTGGAAGTCAAAGAGGGCGCACCACTGCCGTTCAGTTACGAC | |
| sequence | ATCCTCAGTAACGCATTCCAGTACGGTAACCGTGCATTCACAAAATAC | |
| | CCCGACGACATCGCAAACTACTTCAAACAGTCATTCCCAGAGGGTTAC | |
| | AGCTGGGAGCGGACAATGACATTCGAGGACAAAGGGATCGTAAAAGTG | |
| | AAAAGTGACATATCAATGGAAGAGGACTCATTCATCTACGAGATAAGG | |
| | TTAAAAGGGAAGAACTTCCCACCAAACGGTCCAGTGATGCAGAAGAAA | |
| | ACACTCAAATGGGAGCCATCAACCGAGATCCTCTACGTGCGTGACGGT | |
| | GTCTTGGTGGGTGACATCTCACACAGTTTGCTGCTCGAGGGTGGCGGT | |
| | CACTACCGGTGCGACTTCAAAACCATCTACAAAGCCAAGAAAGTAGTC | |
| | AAACTGCCCGACTACCACTTCGTCGACCACAGGATAGAGATCTTGAAC | |
| | CACGACAAAGACTACAACAAAGTCACATTGTACGAGAACGCAGTGGCC | |
| | CGATACAGCCTGTTACCACCACAGGCCGGGATGGACGAGTTGTACAAA | |
| | TGA | |
| Exemplary | ATGGCATTGAGCAAACAGTCCCTACCCAGCGACATGAAATTGATCTAC | 108 |
| meffCFP | CACATGGACGGGAACGTGAACGGTCACTCCTTCGTCATAAAAGGCGAG | |
| reporter | GGTGAGGGTAAACCATACGAGGGCACACACACAATAAAACTGCAGGTA | |
| nucleotide | GTCGAGGGTAGTCCGCTGCCGTTCAGCGCCGACATACTGTCAACCGTA | |
| sequence | TTCCAGTACGGTAACCGATGCTTCACAAAATACCCACCAAACATAGTG | |
| | GACTACTTCAAGAACTCATGCTCCGGTGGTGGCTACAAATTCGGGCGT | |
| | TCATTCCTATACGAGGACGGCGCGGTCTGCACAGCAAGTGGTGACATA | |
| | ACACTCAGTGCAGACAAGAAATCATTCGAGCACAAATCGAAATTCCTG | |
| | GGCGTGAACTTCCCAGCAGACGGCCCGGTGATGAAGAAAGAGACAACA | |
| | AACTGGGAGCCATCATGCGAGAAAATGACGCCCAACGGCATGACGTTG | |
| | ATCGGGGACGTCACAGGCTTCTTATTAAAAGAGGACGGGAAACGGTAC | |
| | AAATGCCAGTTCCACACCTTCCACGACGCCAAAGACAAAAGCAAGAAG | |
| | ATGCCGATGCCAGACTTCCACTTCGTGCAGCACAAAATAGAGCGGAAA | |
| | GACCTGCCAGGTTCAATGCAGACATGGCGACTGACAGAGCACGCAGCC | |
| | GCGTGCAAAACGTGCTTCACCGAGTGA | |
| Exemplary | ATGAGTAAAGGTGAGGAACTGTTCACAGGCGTTGTACCGATCCTGGTG | 109 |
| Folding | GAGTTAGACGGCGACGTGAACGGTCACAAATTCTCAGTCAGTGGTGAG | |
| Reporter | GGTGAGGGCGACGCCACATACGGTAAATTGACACTGAAATTCATATGC | |
| GFP | ACAACAGGTAAATTGCCCGTACCCTGGCCAACGTTGGTAACAACCCTA | |
| reporter | ACGTACGGTGTCCAGTGCTTCTCGCGATACCCAGACCACATGAAACGT | |
| nucleotide | CACGACTTCTTCAAAAGCGCGATGCCAGAGGGTTACGTCCAGGAGCGA | |
| sequence | ACAATATCATTCAAAGACGACGGTAACTACAAAACAAGGGCAGAGGTG | |
| | AAATTCGAGGGTGACACATTAGTCAACCGAATAGAGTTAAAAGGTATC | |
| | GACTTCAAAGAGGACGGTAACATACTAGGTCACAAACTCGAGTACAAC | |
| | TACAACTCCCACAACGTCTACATAACAGCGGACAAACAGAAGAACGGT | |
| | ATCAAAGCAAACTTCAAAATCAGGCACAACATCGAGGACGGCTCAGTG | |
| | CAGCTCGCGGACCACTACCAGCAGAACACACCCATCGGTGACGGTCCG | |
| | GTCTTACTCCCCGACAACCACTACCTATCAACGCAGTCCGCCCTGAGT | |
| | AAAGACCCAAACGAGAAACGTGACCACATGGTCCTACTCGAGTTCGTA | |
| | ACAGCAGCGGGGATAACCCACGGTATGGACGAGTTATACAAATGA | |
| Exemplary | ATGTCCCTCTCGAAACAAGTATTACCAAGAGACGTTAAAATGCGATTC | 110 |
| ccalOFP1 | CACATGGACGGTTGCGTGAACGGCCACTCATTCACGATAGAAGGAGAG | |
| reporter | GGTACCGGGAAACCGTACGAGGGTAAGAAAACGTTGAAACTCAGGGTG | |
| nucleotide | ACAAAAGGTGGTCCGCTACCGTTCGCCTTCGACATCCTGTCGGCGACC | |
| sequence | TTCACGTACGGCAACAGGTGCTTCTGCGACTACCCAGAGGAGATGCCC | |
| | GACTACTTCAAACAGAGTTTACCAGAGGGTTACAGCTGGGAGAGGACG | |
| | ATGATGTACGAGGACGGTGCATGCTCAACAGCGAGTGCCCACATCAGT | |
| | TTGGACAAAGACTGCTTCATCCACAACAGTACATTCCACGGTGTGAAC | |
| | TTCCCAGCGAACGGCCCAGTCATGCAGAAGAAGGCGATGAACTGGGAG | |
| | CCGAGCTCAGAGTTAATAACCCCATGCGACGGGATCTTGAAAGGCGAC | |
| | GTAACGATGTTCTTACTACAAGAGGGTGGTCACCGTCACAAATGCCAG | |
| | TTCACAACTTCCTACAAAGCCCACAAAGCGGTCAAAATCCCGCCAAAC | |
| | CACATCATCGAGCACAGGTTGGTACGTAAAGAGGTGGGTGACGCAGTC | |
| | CAGATCCAGGAGCACGCAGTGGCGAAACACTTCACAGTCCAGATAAAA | |
| | GAGGCGTGA | |
| Exemplary | ATGTCAGAGTTGATAAAAGAGAACATGCACATGAAATTATACATGGAA | 111 |
| tdK atushka2 | GGTACCGTAAACAACCACCACTTCAAATGCACCTCAGAGGGAGAGGGT | |
| reporter | AAACCGTACGAGGGTACACAGACAATGAAAATCAAAGTGGTCGAGGGT | |
| nucleotide | GGTCCCCTACCATTCGCGTTCGACATCCTGGCCACCAGTTTCATGTAC | |
| sequence | GGCTCAAAGACGTTCATAAACCACACACAGGGGATACCCGACTTCTTC | |
| | AAACAGTCATTCCCAGAGGGCTTCACCTGGGAGCGAATCACAACATAC | |
| | GAGGACGGCGGTGTGTTGACAGCAACGCAGGACACATCCCTGCAGAAC | |
| | GGTTGCATAATATACAACGTTAAAATAAACGGTGTCAACTTCCCATCG | |
| | AACGGGAGTGTGATGCAGAAGAAAACCTTAGGTTGGGAAGCCAACACC | |
| | GAGATGTTGTACCCCGCCGACGGCGGCCTACGGGGACACAGTCAGATG | |
| | GCCTTAAAACTAGTGGGTGGTGGTTACCTACACTGCAGTTTCAAAACA | |
| | ACCTACCGTAGCAAGAAACCAGCGAAGAACCTCAAAATGCCAGGTTTC | |
| | CACTTCGTGGACCACCGTCTCGAGAGGATCAAAGAGGCGGACAAAGAG | |
| | ACATACGTGGAGCAGCACGAGATGGCGGTCGCGAAATACTGCGACCTA | |
| | CCATCCAAACTAGGTCACCGTTAG | |
| Exemplary | ATGTCTAAAGGAGAGGAGTTGTTCACTGGTGTCGTGCCGATCCTGGTC | 112 |
| vsfGFP-0 | GAGCTCGACGGTGACGTCAACGGGCACAAATTCTCAGTCCGAGGTGAG | |
| reporter | GGCGAGGGTGACGCAACAAACGGTAAATTGACACTGAAATTCATCTGC | |
| nucleotide | ACGACGGGTAAATTACCGGTACCGTGGCCAACATTGGTGACGACACTG | |
| sequence | ACATACGGTGTGCAGTGCTTCAGCCGATACCCCGACCACATGAAACGA | |
| | CACGACTTCTTCAAATCAGCAATGCCAGAGGGTTACGTACAGGAGAGG | |
| | ACGATCAGCTTCAAAGACGACGGCACCTACAAAACCCGTGCGGAAGTG | |
| | AAATTCGAGGGTGACACCTTGGTCAACCGAATCGAGTTGAAAGGTATC | |
| | GACTTCAAAGAGGACGGTAACATATTAGGTCACAAATTGGAGTACAAC | |
| | TTCAACAGTCACAACGTCTACATCACAGCCGACAAACAGAAGAACGGT | |
| | ATCAAAGCCAACTTCAAAATCCGTCACAACGTAGAGGACGGCTCCGTG | |
| | CAGCTAGCGGACCACTACCAGCAGAACACGCCAATCGGGGACGGCCCC | |
| | GTACTGCTGCCAGACAACCACTACCTATCAACACAGAGCGTGCTCTCA | |
| | AAAGACCCAAACGAGAAACGGGACCACATGGTGTTGTTGGAGTTCGTA | |
| | ACGGCGGCAGGTATAGCGCAGGTGCAGTTGGTAGAGTCAGGTGGGGCA | |
| | TTGGTACAGCCAGGTGGTTCACTGCGGTTATCATGCGCAGCATCAGGT | |
| | TTCCCGGTAAACAGGTACTCCATGCGATGGTACCGGCAGGCACCGGGT | |
| | AAAGAGAGGGAGTGGGTGGCGGGTATGTCCAGTGCGGGTGACAGGTCG | |
| | TCGTACGAGGACTCAGTCAAAGGTAGGTTCACCATAAGTAGGGACGAC | |
| | GCACGAAACACCGTGTACCTGCAGATGAACAGTCTAAAACCAGAGGAC | |
| | ACAGCGGTGTACTACTGCAACGTCAACGTAGGTTTCGAGTACTGGGGT | |
| | CAGGGTACGCAGGTGACAGTGTCGTGA | |
| Exemplary | ATGACCACATTCAAAATCGAGAGTAGGATCCACGGTAACTTGAACGGC | 113 |
| eYGFPuv | GAGAAATTCGAGCTAGTAGGCGGTGGTGTAGGGGAAGAGGGAAGGCTC | |
| reporter | GAGATCGAGATGAAAACAAAAGACAAACCGTTAGCATTCTCGCCATTC | |
| nucleotide | CTGTTGACAACGTGCATGGGTTACGGTTTCTACCACTTCGCTTCCTTC | |
| sequence | CCGAAAGGTATAAAGAACATATACTTGCACGCAGCCACGAACGGGGC | |
| | TACACCAACACACGTAAAGAGATATACGAGGACGGTGGTATACTGGAA | |
| | GTCAACTTCAGGTACACGTACGAGTTCAACAAAATCATCGGCGACGTG | |
| | GAGTGCATAGGTCACGGCTTCCCCTCGCAGTCCCCAATCTTCAAAGAC | |
| | ACAATAGTCAAATCGTGCCCAACGGTGGACTTAATGCTGCCAATGAGC | |
| | GGGAACATAATCGCCTCATCCTACGCATACGCATTCCAGCTCAAAGAC | |
| | GGTAGTTTCTACACAGCCGAGGTCAAGAACAACATAGACTTCAAGAAC | |
| | CCAATACACGAGTCCTTCTCAAAATCCGGGCCGATGTTCACACACCGT | |
| | CGGGTTGAGGAGACACTAACAAAAGAGAACCTGGCAATAGTGGAGTAC | |
| | CAGCAGGTGTTCAACTCGGCCCCGCGGGACATGTGA | |

TABLE 11
Exemplary Reporter Polypeptide Sequences

| | | SEQ ID |
|---|---|---|
| Description | Sequence | NO: |
| Exemplary | MVSKGEELFTGVVPILVELDGDVNGHKFSVSGEGEGDATYGKLTLKFI | 114 |
| eGFP | CTTGKLPVPWPTLVTTLTYGVQCFSRYPDHMKQHDFFKSAMPEGYVQE | |
| reporter | RTIFFKDDGNYKTRAEVKFEGDTLVNRIELKGIDFKEDGNILGHKLEY | |
| amino acid | NYNSHNVYIMADKQKNGIKVNFKIRHNIEDGSVQLADHYQQNTPIGDG | |
| sequence | PVLLPDNHYLSTQSALSKDPNEKRDHMVLLEFVTAAGITLGMDELYK | |
| Exemplary | MVSKGEEDNMAIIKEFMRFKVHMEGSVNGHEFEIEGEGEGRPYEGTQT | 115 |
| mCherry | AKLKVTKGGPLPFAWDILSPQFMYGSKAYVKHPADIPDYLKLSFPEGF | |
| reporter | KWERVMNFEDGGVVTVTQDSSLQDGEFIYKVKLRGTNFPSDGPVMQKK | |
| amino acid | TMGWEASSERMYPEDGALKGEIKQRLKLKDGGHYDAEVKTTYKAKKPV | |
| sequence | QLPGAYNVNIKLDITSHNEDYTIVEQYERAEGRHSTGGMDELYK | |
| Exemplary | MVSKGEELIKENMRMKVVMEGSVNGHQFKCTGEGEGNPYMGTQTMRIK | 116 |
| mRuby | VIEGGPLPFAFDILATSFMYGSRTFIKYPKGIPDFFKQSFPEGFTWER | |
| reporter | VTRYEDGGVVTVMQDTSLEDGCLVYHVQVRGVNFPSNGPVMQKKTKGW | |
| amino acid | EPNTEMMYPADGGLRGYTHMALKVDGGGHLSCSFVTTYRSKKTVGNIK | |
| sequence | MPGIHAVDHRLERLEESDNEMFVVQREHAVAKFAGLGGGMDELYK | |
| Exemplary | MVSKGEEVIKEFMRFKVRMEGSMNGHEFEIEGEGEGRPYEGTQTAKLK | 117 |
| RRvT | VTKGGPLPFAWDILSPQFMYGSKAYVKHPADIPDYKKLSFPEGFKWER | |
| reporter | VMNFEDGGLVTVTQDSSLQDGTLIYNVKMRGTNFPPDGPVMQKKTMGW | |
| amino acid | EASTERLYPRDGVLKGEIHQALKLKDGGHYLVEFKTIYMAKKPVQLPG | |
| sequence | YYYVDTKLDITSHNEDYTIVEQYERSEGRHHLFLYGMDELYK | |
| Exemplary | MVSKGEETTMGVIKPDMKIKLKMEGNVNGHAFVIEGEGEGKPYDGTNT | 118 |
| mTFP1 | INLEVKEGAPLPFSYDILTTAFAYGNRAFTKYPDDIPNYFKQSFPEGY | |
| reporter | SWERTMTFEDKGIVKVKSDISMEEDSFIYEIHLKGENFPPNGPVMQKK | |
| amino acid | TTGWDASTERMYVRDGVLKGDVKHKLLLEGGGHHRVDFKTIYRAKKAV | |
| sequence | KLPDYHFVDHRIEILNHDKDYNKVTVYESAVARNSTDGMDELYK | |
| Exemplary | MNSLIKENMRMMVVMEGSVNGYQFKCTGEGDGNPYMGTQTMRIKVVEG | 119 |
| RFP611 | GPLPFAFDVLATSFMYGSKTFIKHTKGIPDFFKQSFPEGFTWERVTRY | |
| reporter | EDGGVITVMQDTSLEDGCLVYHAKVTGVNFPSNGAVMQKKTKGWEPNT | |
| amino acid | EMLYPADGGLRGYSQMALNVDGGGYLSCSFETTYRSKKTVENFKMPGF | |
| sequence | HFVDHRLERLEESDKEMFVVQHEHAVAKFCDLPSKLGRL | |
| Exemplary | MVSKGEETTMGVIKPDMKIKLKMEGNVNGHAFVIEGEGEGKPYDGTNT | 120 |
| dTFP0.2 | VNLEVKEGAPLPFSYDILSNAFQYGNRAFTKYPDDIANYFKQSFPEGY | |
| reporter | SWERTMTFEDKGIVKVKSDISMEEDSFIYEIRLKGKNFPPNGPVMQKK | |
| amino acid | TLKWEPSTEILYVRDGVLVGDISHSLLLEGGGHYRCDFKTIYKAKKVV | |
| sequence | KLPDYHFVDHRIEILNHDKDYNKVTLYENAVARYSLLPPQAGMDELYK | |
| Exemplary | MALSKQSLPSDMKLIYHMDGNVNGHSFVIKGEGEGKPYEGTHTIKLQV | 121 |
| meffCFP | VEGSPLPFSADILSTVFQYGNRCFTKYPPNIVDYFKNSCSGGGYKFGR | |
| reporter | SFLYEDGAVCTASGDITLSADKKSFEHKSKFLGVNFPADGPVMKKETT | |
| amino acid | NWEPSCEKMTPNGMTLIGDVTGFLLKEDGKRYKCQFHTFHDAKDKSKK | |
| sequence | MPMPDFHFVQHKIERKDLPGSMQTWRLTEHAAACKTCFTE | |
| Exemplary | MSKGEELFTGVVPILVELDGDVNGHKFSVSGEGEGDATYGKLTLKFIC | 122 |
| Folding | TTGKLPVPWPTLVTTLTYGVQCFSRYPDHMKRHDFFKSAMPEGYVQER | |
| Reporter | TISFKDDGNYKTRAEVKFEGDTLVNRIELKGIDFKEDGNILGHKLEYN | |
| GFP | YNSHNVYITADKQKNGIKANFKIRHNIEDGSVQLADHYQQNTPIGDGP | |
| reporter | VLLPDNHYLSTQSALSKDPNEKRDHMVLLEFVTAAGITHGMDELYK | |
| amino acid | | |
| sequence | | |
| Exemplary | MSLSKQVLPRDVKMRFHMDGCVNGHSFTIEGEGTGKPYEGKKTLKLRV | 123 |
| ccalOFP1 | TKGGPLPFAFDILSATFTYGNRCFCDYPEEMPDYFKQSLPEGYSWERT | |
| reporter | MMYEDGACSTASAHISLDKDCFIHNSTFHGVNFPANGPVMQKKAMNWE | |
| amino acid | PSSELITPCDGILKGDVTMFLLQEGGHRHKCQFTTSYKAHKAVKIPPN | |
| sequence | HIIEHRLVRKEVGDAVQIQEHAVAKHFTVQIKEA | |
| Exemplary | MSELIKENMHMKLYMEGTVNNHHFKCTSEGEGKPYEGTQTMKIKVVEG | 124 |
| tdKatushka2 | GPLPFAFDILATSFMYGSKTFINHTQGIPDFFKQSFPEGFTWERITTY | |
| reporter | EDGGVLTATQDTSLQNGCIIYNVKINGVNFPSNGSVMQKKTLGWEANT | |
| amino acid | EMLYPADGGLRGHSQMALKLVGGGYLHCSFKTTYRSKKPAKNLKMPGF | |
| sequence | HFVDHRLERIKEADKETYVEQHEMAVAKYCDLPSKLGHR | |
| Exemplary | MSKGEELFTGVVPILVELDGDVNGHKFSVRGEGEGDATNGKLTLKFIC | 125 |
| vsfGFP-0 | TTGKLPVPWPTLVTTLTYGVQCFSRYPDHMKRHDFFKSAMPEGYVQER | |
| reporter | TISFKDDGTYKTRAEVKFEGDTLVNRIELKGIDFKEDGNILGHKLEYN | |
| amino acid | FNSHNVYITADKQKNGIKANFKIRHNVEDGSVQLADHYQQNTPIGDGP | |
| sequence | VLLPDNHYLSTQSVLSKDPNEKRDHMVLLEFVTAAGIAQVQLVESGGA | |
| | LVQPGGSLRLSCAASGFPVNRYSMRWYRQAPGKEREWVAGMSSAGDRS | |
| | SYEDSVKGRFTISRDDARNTVYLQMNSLKPEDTAVYYCNVNVGFEYWG | |
| | QGTQVTVS | |
| Exemplary | MTTFKIESRIHGNLNGEKFELVGGGVGEEGRLEIEMKTKDKPLAFSPF | 126 |
| eYGFPuv | LLTTCMGYGFYHFASFPKGIKNIYLHAATNGGYTNTRKEIYEDGGILE | |
| reporter | VNFRYTYEFNKIIGDVECIGHGFPSQSPIFKDTIVKSCPTVDLMLPMS | |
| amino acid | GNIIASSYAYAFQLKDGSFYTAEVKNNIDFKNPIHESFSKSGPMFTHR | |
| sequence | RVEETLTKENLAIVEYQQVFNSAPRDM | |

### Exemplary Coding Sequence

In some embodiments, a construct of the present disclosure comprises a coding sequence that is at least 85%, at least 90%, at least 91%, at least 92%, at least 93%, at least 94%, at least 95%, at least 96%, at least 97%, at least 98%, or at least 99% identical to a sequence included in Table 12. In some embodiments, a coding sequence described herein has a polynucleotide sequence provided in Table 12.

TABLE 12
Exemplary Coding Sequence Polynucleotide Sequences

| | | SEQ ID |
|---|---|---|
| Description | Sequence | NO: |
| ZmUbi - | CTGCAGTGCAGCGTGACCCGGTCGTGCCCCTCTCTAGAGATAATGAGC | 155 |
| CYP76AD1 - | ATTGCATGTCTAAGTTATAAAAAATTACCACATATTTTTTTTGTCACA | |
| P2A - | CTTGTTTGAAGTGCAGTTTATCTATCTTTATACATATATTTAAACTTT | |
| DODA - | ACTCTACGAATAATATAATCTATAGTACTACAATAATATCAGTGTTTT | |
| P2A - | AGAGAATCATATAAATGAACAGTTAGACATGGTCTAAAGGACAATTGA | |
| cDOPA5GT | GTATTTTGACAACAGGACTCTACAGTTTTATCTTTTTAGTGTGCATGT | |
| | GTTCTCCTTTTTTTTTGCAAATAGCTTCACCTATATAATACTTCATCC | |
| | ATTTTATTAGTACATCCATTTAGGGTTTAGGGTTAATGGTTTTTATAG | |
| | ACTAATTTTTTTAGTACATCTATTTTATTCTATTTTAGCCTCTAAATT | |
| | AAGAAAACTAAAACTCTATTTTAGTTTTTTTATTTAATAATTTAGATA | |
| | TAAAATAGAATAAAATAAAGTGACTAAAAATTAAACAAATACCCTTTA | |
| | AGAAATTAAAAAAACTAAGGAAACATTTTTCTTGTTTCGAGTAGATAA | |
| | TGCCAGCCTGTTAAACGCCGTCGACGAGTCTAACGGACACCAACCAGC | |
| | GAACCAGCAGCGTCGCGTCGGGCCAAGCGAAGCAGACGGCACGGCATC | |
| | TCTGTCGCTGCCTCTGGACCCCTCTCGAGAGTTCCGCTCCACCGTTGG | |
| | ACTTGCTCCGCTGTCGGCATCCAGAAATTGCGTGGCGGAGCGGCAGAC | |
| | GTGAGCCGGCACGGCAGGCGGCCTCCTCCTCCTCTCACGGCACCGGCA | |
| | GCTACGGGGGATTCCTTTCCCACCGCTCCTTCGCTTTCCCTTCCTCGC | |
| | CCGCCGTAATAAATAGACACCCCCTCCACACCCTCTTTCCCCAACCTC | |
| | GTGTTGTTCGGAGCGCACACACACACAACCAGATCTCCCCCAAATCCA | |
| | CCCGTCGGCACCTCCGCTTCAAGGTACGCCGCTCGTCCTCCCCCCCCC | |
| | CCCTCTCTACCTTCTCTAGATCGGCGTTCCGGTCCATGGTTAGGGCCC | |
| | GGTAGTTCTACTTCTGTTCATGTTTGTGTTAGATCCGTGTTTGTGTTA | |
| | GATCCGTGCTGCTAGCGTTCGTACACGGATGCGACCTGTACGTCAGAC | |
| | ACGTTCTGATTGCTAACTTGCCAGTGTTTCTCTTTGGGGAATCCTGGG | |
| | ATGGCTCTAGCCGTTCCGCAGACGGGATCGATTTCATGATTTTTTTTG | |
| | TTTCGTTGCATAGGGTTTGGTTTGCCCTTTTCCTTTATTTCAATATAT | |
| | GCCGTGCACTTGTTTGTCGGGTCATCTTTTCATGCTTTTTTTTGTCTT | |
| | GGTTGTGATGATGTGGTCTGGTTGGGCGGTCGTTCTAGATCGGAGTAG | |
| | AATTCTGTTTCAAACTACCTGGTGGATTTATTAATTTTGGATCTGTAT | |
| | GTGTGTGCCATACATATTCATAGTTACGAATTGAAGATGATGGATGGA | |
| | AATATCGATCTAGGATAGGTATACATGTTGATGCGGGTTTTACTGATG | |
| | CATATACAGAGATGCTTTTTGTTCGCTTGGTTGTGATGATGTGGTGTG | |
| | GTTGGGCGGTCGTTCATTCGTTCTAGATCGGAGTAGAATACTGTTTCA | |
| | AACTACCTGGTGTATTTATTAATTTTGGAACTGTATGTGTGTGTCATA | |
| | CATCTTCATAGTTACGAGTTTAAGATGGATGGAAATATCGATCTAGGA | |
| | TAGGTATACATGTTGATGTGGGTTTTACTGATGCATATACATGATGGC | |
| | ATATGCAGCATCTATTCATATGCTCTAACCTTGAGTACCTATCTATTA | |
| | TAATAAACAAGTATGTTTTATAATTATTTTGATCTTGATATACTTGGA | |
| | TGATGGCATATGCAGCAGCTATATGTGGATTTTTTTAGCCCTGCCTTC | |
| | ATACGCTATTTATTTGCTTGGTACTGTTTCTTTTGTCGATGCTCACCC | |
| | TGTTGTTTGGTGTTACTTCTGCAGAATGGATCATGCGACCCTCGCCAT | |
| | GATCCTCGCGATCTGGTTCATCAGCTTCCACTTCATCAAGCTGCTGTT | |
| | CTCCCAGCAGACCACCAAGCTGCTTCCGCCAGGACCAAAGCCGCTTCC | |
| | GATCATCGGCAACATCCTTGAGGTGGGCAAGAAGCCGCATCGGTCCTT | |
| | CGCCAACCTCGCCAAGATTCACGGCCCACTCATTTCCCTCAGACTCGG | |
| | CTCTGTGACCACCATCGTTGTGTCCTCTGCCGACGTGGCCAAAGAGAT | |
| | GTTCCTCAAGAAGGATCACCCGCTCTCCAACCGCACGATCCCGAATAG | |
| | TGTTACAGCCGGCGACCACCACAAGCTCACCATGTCTTGGCTCCCGGT | |
| | GTCTCCGAAGTGGCGCAACTTCCGCAAGATTACCGCCGTGCATCTGCT | |
| | CTCCCCACAGAGACTCGATGCCTGCCAGACATTCAGGCACGCCAAGGT | |
| | GCAGCAGCTCTACGAGTACGTTCAAGAGTGCGCCCAGAAAGGCCAGGC | |
| | CGTGGATATTGGCAAGGCCGCCTTTACGACCAGCCTCAACCTCCTCAG | |
| | CAAGCTGTTCTTCAGCGTCGAGCTGGCGCACCACAAGTCCCATACCAG | |
| | CCAAGAGTTCAAAGAGCTGATCTGGAACATCATGGAAGATATAGGCAA | |
| | GCCGAACTACGCCGACTACTTCCCGATTCTCGGCTGCGTTGACCCATC | |
| | TGGCATTAGAAGAAGGCTCGCCTGCTCCTTCGACAAGCTGATCGCCGT | |
| | GTTCCAGGGCATCATCTGCGAGAGACTCGCCCCAGATTCCTCCACCAC | |
| | AACTACCACCACCACCGACGACGTGCTCGATGTGCTCCTCCAGCTGTT | |
| | CAAGCAGAACGAGCTGACGATGGGCGAGATCAACCACCTCCTCGTGGA | |
| | CATCTTCGACGCCGGCACCGATACCACATCCTCCACATTCGAGTGGGT | |
| | GATGACCGAGCTGATCCGCAATCCAGAGATGATGGAAAAGGCCCAAGA | |
| | GGAAATCAAGCAGGTCCTCGGCAAGGACAAGCAGATCCAAGAGTCCGA | |
| | CATCATCAACCTGCCGTACCTCCAGGCGATCATCAAAGAGACACTCCG | |
| | CCTCCATCCGCCGACCGTGTTCTTGCTCCCAAGAAAGGCCGACACCGA | |
| | TGTCGAGCTGTACGGCTACATCGTGCCGAAGGATGCCCAGATCCTCGT | |
| | GAACCTCTGGGCCATTGGCAGGGACCCAAACGCCTGGCAGAACGCCGA | |
| | TATTTTCAGCCCAGAGCGCTTCATCGGCTGCGAGATCGATGTTAAGGG | |
| | CCGCGATTTCGGCCTCCTTCCATTTGGCGCTGGCCGCAGAATTTGCCC | |
| | AGGCATGAATCTCGCCATCAGGATGCTCACCCTCATGCTCGCCACACT | |
| | CCTCCAGTTCTTCAACTGGAAGCTCGAAGGCGACATCTCCCCGAAGGA | |
| | CCTCGACATGGACGAGAAGTTCGGCATTGCGCTCCAAAAGACCAAGCC | |
| | GCTCAAGCTCATCCCGATTCCGCGCTACGGTAGCGGAGCTACCAATTT | |
| | TAGCCTCCTTAAGCAGGCAGGTGATGTAGAAGAGAACCCCGGGCCTAT | |
| | GAAGATGATGAACGGCGAGGACGCCAACGACCAGATGATCAAAGAGTC | |
| | CTTCTTCATCACCCACGGCAACCCGATCCTCACCGTCGAGGATACACA | |
| | TCCGCTCAGGCCGTTCTTCGAGACATGGCGCGAGAAGATTTTCTCCAA | |
| | GAAGCCGAAGGCCATCCTCATCATCTCCGGCCACTGGGAGACAGTGAA | |
| | GCCAACCGTGAACGCCGTGCACATCAACGACACCATCCACGACTTCGA | |
| | CGACTACCCAGCCGCCATGTACCAGTTCAAGTACCCAGCTCCAGGCGA | |
| | GCCAGAGCTTGCGAGAAAGGTGGAAGAGATCCTCAAGAAGTCCGGGTT | |
| | CGAGACAGCCGAGACAGACCAAAAGAGGGGCCTTGATCACGGCGCCTG | |
| | GGTTCCACTCATGCTCATGTATCCAGAGGCGGACATCCCGGTGTGCCA | |
| | GCTCTCAGTTCAGCCACATCTCGACGGCACCTACCACTACAATCTCGG | |
| | CAGAGCCCTCGCGCCGCTCAAGAATGATGGCGTGCTCATTATTGGCTC | |
| | CGGCAGCGCCACACATCCACTCGATGAGACACCGCACTACTTCGATGG | |
| | TGTTGCCCCTTGGGCCGCTGCCTTCGATTCTTGGCTTAGGAAGGCCCT | |
| | CATCAACGGCCGCTTCGAGGAAGTGAACATCTACGAGAGCAAGGCCCC | |
| | GAACTGGAAGCTCGCCCATCCATTTCCAGAGCACTTCTACCCGCTCCA | |
| | CGTTGTGCTCGGCGCTGCTGGTGAAAAGTGGAAGGCCGAGCTGATCCA | |
| | CTCCTCCTGGGATCATGGCACACTTTGCCACGGCTCCTACAAGTTCAC | |
| | CTCCGCCGGATCCGGAGCAACCAACTTTAGCCTGCTCAAGCAAGCAGG | |
| | AGATGTTGAGGAAAATCCTGGCCCCATGACCGCCATCAAGATGAACAC | |
| | CAACGGCGAGGGCGAGACACAGCACATCCTCATGATCCCGTTCATGGC | |
| | GCAGGGCCACCTCAGGCCATTTCTCGAACTCGCCATGTTCCTCTACAA | |
| | GCGCTCCCACGTGATCATCACCCTGCTCACAACTCCGCTCAATGCCGG | |
| | CTTCCTCAGGCACCTCCTTCACCACCATTCCTACTCCTCCAGCGGCAT | |
| | CAGGATCGTCGAGCTGCCATTCAACTCCACCAACCACGGACTCCCACC | |
| | GGGCATCGAGAACACCGATAAGCTCACACTCCCGCTCGTGGTGTCCCT | |
| | CTTCCATTCCACCATCAGCCTCGATCCGCACCTCCGCGATTACATCTC | |
| | CAGGCATTTCAGCCCAGCCAGGCCACCACTCTGCGTGATCCATGATGT | |
| | GTTCCTCGGCTGGGTTGACCAGGTGGCCAAGGATGTGGGCTCTACAGG | |
| | CGTGGTGTTCACAACAGGCGGCGCTTATGGCACATCCGCCTACGTGTC | |
| | CATCTGGAACGATCTCCCGCACCAGAACTACTCCGACGACCAAGAGTT | |
| | CCCGCTGCCAGGCTTCCCAGAGAACCATAAGTTCCGCAGGTCCCAGCT | |
| | CCATCGGTTCCTCAGATATGCCGACGGCTCCGACGATTGGTCCAAGTA | |
| | TTTCCAGCCGCAGCTCCGCCAGTCCATGAAGTCTTTTGGCTGGCTCTG | |
| | CAACTCCGTGGAAGAGATCGAGACACTCGGCTTCTCCATCCTCCGCAA | |
| | CTACACCAAGCTGCCGATCTGGGGCATCGGCCCACTTATTGCTTCCCC | |
| | AGTGCAGCACTCCTCCTCCGACAACAATTCAACAGGCGCCGAGTTCGT | |
| | GCAGTGGCTCAGCCTCAAAGAGCCGGACTCCGTCCTCTACATCTCCTT | |
| | CGGCTCCCAGAACACGATCAGCCCGACGCAGATGATGGAACTCGCTGC | |
| | TGGCCTTGAGTCCTCCGAGAAGCCATTCCTCTGGGTGATCAGAGCCCC | |
| | GTTCGGCTTCGACATCAACGAAGAGATGCGCCCAGAGTGGCTGCCAGA | |
| | GGGCTTTGAGGAACGCATGAAGGTGAAGAAACAGGGCAAGCTCGTGTA | |
| | CAAGCTCGGCCCGCAGCTTGAGATCCTCAACCATGAATCCATCGGCGG | |
| | CTTTCTCACCCACTGCGGATGGAACAGCATCCTTGAGTCTCTTCGCGA | |
| | GGGCGTTCCGATGCTTGGATGGCCACTTGCTGCCGAGCAGGCCTACAA | |
| | CCTCAAGTACCTCGAAGATGAGATGGGCGTCGCGGTTGAGCTTGCTAG | |
| | AGGCCTCGAAGGCGAGATCTCCAAAGAGAAGGTCAAGCGCATCGTCGA | |
| | GATGATCCTTGAGCGCAACGAGGGCTCCAAAGGCTGGGAGATGAAGAA | |
| | TCGCGCCGTGGAAATGGGCAAAAAGCTCAAGGACGCCGTGAACGAGGA | |
| | AAAAGAGCTGAAGGGCTCCTCCGTGAAGGCGATCGACGATTTCCTCGA | |
| | CGCCGTCATGCAGGCCAAACTTGAGCCAAGCCTCCAGTGA | |
| BdUbi10- | GAAGAACTCGAGAGGGAATTGCAGATCATGAGGCAGATGGCTATTTTT | 156 |
| CYP76AD1 - | GTGTCACATATGCGCAAAAAGAGAGGCTATATTTGTGTCCCTAGGTTC | |
| P2A - | TTCGTTGTATTGCAGTTTCCATATCAATCTGACTTGGTCGCATGAGAA | |
| DODA - | ATTGATGGTTAAATAATTTGAATCTCTCATGTAGTATCAACTATTAGA | |
| P2A - | TATTATTTTCACCAAATATATTTCCATCGGAGAAGAAGAGGCTACAGA | |
| cDOPA5GT | GGAAGCAGAAGAGAGGGGTGGGAGAATTTTTACACTTTTGTACACCCA | |
| | CTTAAACAGCAAAATCCGTATGAAAACAGGCCCACCAAAACAATGCCA | |
| | CGATAACAATCCGTAGAAACAAAAGCTTCATTTAACAGCGGCGCAACA | |
| | AAGCACGCTTATCCATGGTAGTTGTAGTCCGTATGCGATCCAAAGATC | |
| | ACGATTCACGCGTGACGGACGGACGACGCGTGCCACACCACAACTAAC | |
| | GGCATCCATGGTAGTTGTAGTCCGTATGCGATCCAAAGATCACGATTC | |
| | ACGCGTGACGGACGGACGACGCGCGCCACACCACAACTAACAGCGTGA | |
| | GCCAGCGTCCAAACTCCGGATGGCAACGGGGACGAAACCCGTCGGGTA | |
| | GTCACTGCCCAAACCCGTCCCCGCAACCTTCATCCCAAACCCGTCCCC | |
| | GTTTCCGGTCGCGGGTTTCAGTTTTCTACCAGACCCGTCCCCATCGGG | |
| | TTTTTCATCCCCGTCGGGAAATCCGAACCCGCCAGCATTTCAGCACCA | |
| | AGCCAAAGTTGCAGCAGCAACATGAATAAAAAACAACCCGTTTCAACA | |
| | CCAAGATAAAACAAAACATTATAATTTAGACAACATTTCACACGTATA | |
| | ACAATAACATATAGTTCTCACATATAACAACACCATTTCACACATAAA | |
| | ACAACACCATTTGGGATAAAAATATGGGCTATATCAGGCCATTTTTAT | |
| | GGGCCATATTGAGTTTTCGTGGGTTTCACAGGTACCGGATTTGTAGAA | |
| | TGCTGAACCGGGTTTGAACCGTAAAATCCGCGGGTATTGAATTTGACC | |
| | CAATCCCGTCGTCCCCTGGTGGGGTAAAAACACCATCTTGAGTCCAAA | |
| | CGGCCACCAACCAAACTCCGACGGCAACAAACAAACGGCGTTGCTTTG | |
| | CTCCTCGGTATCTCCGTGACCGCTCAATCTCCCGGCTGTTTCCCCGGA | |
| | ATTGCGTGGACTCTCTCATCCACACGCAAACCGCCTCTCCCTCCTCTC | |
| | TCGTCCTATCCGCCCCGGTGCCGTAGCCTCACGGGACTCTTCTTCCTC | |
| | CCTTGCTATAAAATCCCCGCCCCCTCCCGTCTCCTCTCCACACATCCA | |
| | AACTCTCAATCGCACCGAGAAAAATCTCCTAGCGATCGAAGCGAAGCC | |
| | TCTCCCGATCCTCTCAAGGTACGCCCGTTTCCCGTCGATCCTCCTCCT | |
| | TCCGTTCGTGTTCTGTAGCCGATCGATTCGATTCCCTTACACCCGTTC | |
| | GTGTTCTCTCGTGGATCGATCGATTGTTTGTTGCTAGAAGGAACTCGT | |
| | AGATCTGGCGTTTATGAACTGTGATTCGGGTTAGTCCAGATCGATTCA | |
| | GGTCGGTCGTCGTTGAGCCTCTCGGCTATGTCTGGATTATCGTGTAGA | |
| | TCTGCTGGTTCAGTTGATTATGTTCTTCTAGGAGTAATTTCGTTGGGT | |
| | CAGCGCGATTTCTGCTTAATCTATGCTGCTTATTGCGCCTGTACCTAT | |
| | CTACTAAGCTATGTGCACCTGTAATTTTGCTAGATTATTCGTTCATCC | |
| | TCGTAGTTGGTTTGTCACAGTAATCCGTATGGGTTCTGACGATGTTAT | |
| | TGTTGGTCATACCTAGGCTTCTCCAGATTTTATTTTGTTAAAATTGGA | |
| | TAGATCTGCTACTGATAGTTGATGATGGAATTTGGTGCTGAATCTATG | |
| | CTATTTATTGCGCCTATACCTGATCTATCGGGCTATGTACGGCTGTAG | |
| | TTTACTGGATTATTCGTTCATCCTCGGTAGTTGGTTCATCGTTTGGGT | |
| | TCTGACGATAATATTGTTGATTATGCGTAGGCTTCTGCAGATTGTTGT | |
| | TAAAATTGGATACATCGGTTACTGATGGTTGATGATAGATTTGTGCTG | |
| | AACCTATCTGTTTATTGCTCCTATACCTGATCTATAGGGCTATGTATG | |
| | CCTGTAATTTACCAGATTATTCGTTCATCCTCGTAGTTGGTTCATCTC | |
| | TATAATTCGTATGGGTTCTTATGATGTTATCGTTGATTATGCCTAGTC | |
| | TTATACAGATTATTGTGTCAAGATTGAATATACCTGCTACTGATCGGT | |
| | GATAATTTGGTTAGTAGTTTGCAATCTGCTAGGAACACGTTACCACTG | |
| | TAATCTGTAAACATGGTTTGCCAGAGTAGTTTGTTCTACTACTCTTGA | |
| | TATGGTTGCTGATTTTAGTCGCCTCCTTTTGGATCATGTATTGATGTC | |
| | CTTGCAGATTTCCGTGTACTTACCCCGGCTTTTGTGTACTTCGTGTTA | |
| | ACAGAATGGATCATGCGACCCTCGCCATGATCCTCGCGATCTGGTTCA | |
| | TCAGCTTCCACTTCATCAAGCTGCTGTTCTCCCAGCAGACCACCAAGC | |
| | TGCTTCCGCCAGGACCAAAGCCGCTTCCGATCATCGGCAACATCCTTG | |
| | AGGTGGGCAAGAAGCCGCATCGGTCCTTCGCCAACCTCGCCAAGATTC | |
| | ACGGCCCACTCATTTCCCTCAGACTCGGCTCTGTGACCACCATCGTTG | |
| | TGTCCTCTGCCGACGTGGCCAAAGAGATGTTCCTCAAGAAGGATCACC | |
| | CGCTCTCCAACCGCACGATCCCGAATAGTGTTACAGCCGGCGACCACC | |
| | ACAAGCTCACCATGTCTTGGCTCCCGGTGTCTCCGAAGTGGCGCAACT | |
| | TCCGCAAGATTACCGCCGTGCATCTGCTCTCCCCACAGAGACTCGATG | |
| | CCTGCCAGACATTCAGGCACGCCAAGGTGCAGCAGCTCTACGAGTACG | |
| | TTCAAGAGTGCGCCCAGAAAGGCCAGGCCGTGGATATTGGCAAGGCCG | |
| | CCTTTACGACCAGCCTCAACCTCCTCAGCAAGCTGTTCTTCAGCGTCG | |
| | AGCTGGCGCACCACAAGTCCCATACCAGCCAAGAGTTCAAAGAGCTGA | |
| | TCTGGAACATCATGGAAGATATAGGCAAGCCGAACTACGCCGACTACT | |
| | TCCCGATTCTCGGCTGCGTTGACCCATCTGGCATTAGAAGAAGGCTCG | |
| | CCTGCTCCTTCGACAAGCTGATCGCCGTGTTCCAGGGCATCATCTGCG | |
| | AGAGACTCGCCCCAGATTCCTCCACCACAACTACCACCACCACCGACG | |
| | ACGTGCTCGATGTGCTCCTCCAGCTGTTCAAGCAGAACGAGCTGACGA | |
| | TGGGCGAGATCAACCACCTCCTCGTGGACATCTTCGACGCCGGCACCG | |
| | ATACCACATCCTCCACATTCGAGTGGGTGATGACCGAGCTGATCCGCA | |
| | ATCCAGAGATGATGGAAAAGGCCCAAGAGGAAATCAAGCAGGTCCTCG | |
| | GCAAGGACAAGCAGATCCAAGAGTCCGACATCATCAACCTGCCGTACC | |
| | TCCAGGCGATCATCAAAGAGACACTCCGCCTCCATCCGCCGACCGTGT | |
| | TCTTGCTCCCAAGAAAGGCCGACACCGATGTCGAGCTGTACGGCTACA | |
| | TCGTGCCGAAGGATGCCCAGATCCTCGTGAACCTCTGGGCCATTGGCA | |
| | GGGACCCAAACGCCTGGCAGAACGCCGATATTTTCAGCCCAGAGCGCT | |
| | TCATCGGCTGCGAGATCGATGTTAAGGGCCGCGATTTCGGCCTCCTTC | |
| | CATTTGGCGCTGGCCGCAGAATTTGCCCAGGCATGAATCTCGCCATCA | |
| | GGATGCTCACCCTCATGCTCGCCACACTCCTCCAGTTCTTCAACTGGA | |
| | AGCTCGAAGGCGACATCTCCCCGAAGGACCTCGACATGGACGAGAAGT | |
| | TCGGCATTGCGCTCCAAAAGACCAAGCCGCTCAAGCTCATCCCGATTC | |
| | CGCGCTACGGTAGCGGAGCTACCAATTTTAGCCTCCTTAAGCAGGCAG | |
| | GTGATGTAGAAGAGAACCCCGGGCCTATGAAGATGATGAACGGCGAGG | |
| | ACGCCAACGACCAGATGATCAAAGAGTCCTTCTTCATCACCCACGGCA | |
| | ACCCGATCCTCACCGTCGAGGATACACATCCGCTCAGGCCGTTCTTCG | |
| | AGACATGGCGCGAGAAGATTTTCTCCAAGAAGCCGAAGGCCATCCTCA | |
| | TCATCTCCGGCCACTGGGAGACAGTGAAGCCAACCGTGAACGCCGTGC | |
| | ACATCAACGACACCATCCACGACTTCGACGACTACCCAGCCGCCATGT | |
| | ACCAGTTCAAGTACCCAGCTCCAGGCGAGCCAGAGCTTGCGAGAAAGG | |
| | TGGAAGAGATCCTCAAGAAGTCCGGGTTCGAGACAGCCGAGACAGACC | |
| | AAAAGAGGGGCCTTGATCACGGCGCCTGGGTTCCACTCATGCTCATGT | |
| | ATCCAGAGGCGGACATCCCGGTGTGCCAGCTCTCAGTTCAGCCACATC | |
| | TCGACGGCACCTACCACTACAATCTCGGCAGAGCCCTCGCGCCGCTCA | |
| | AGAATGATGGCGTGCTCATTATTGGCTCCGGCAGCGCCACACATCCAC | |
| | TCGATGAGACACCGCACTACTTCGATGGTGTTGCCCCTTGGGCCGCTG | |
| | CCTTCGATTCTTGGCTTAGGAAGGCCCTCATCAACGGCCGCTTCGAGG | |
| | AAGTGAACATCTACGAGAGCAAGGCCCCGAACTGGAAGCTCGCCCATC | |
| | CATTTCCAGAGCACTTCTACCCGCTCCACGTTGTGCTCGGCGCTGCTG | |
| | GTGAAAAGTGGAAGGCCGAGCTGATCCACTCCTCCTGGGATCATGGCA | |
| | CACTTTGCCACGGCTCCTACAAGTTCACCTCCGCCGGATCCGGAGCAA | |
| | CCAACTTTAGCCTGCTCAAGCAAGCAGGAGATGTTGAGGAAAATCCTG | |
| | GCCCCATGACCGCCATCAAGATGAACACCAACGGCGAGGGCGAGACAC | |
| | AGCACATCCTCATGATCCCGTTCATGGCGCAGGGCCACCTCAGGCCAT | |
| | TTCTCGAACTCGCCATGTTCCTCTACAAGCGCTCCCACGTGATCATCA | |
| | CCCTGCTCACAACTCCGCTCAATGCCGGCTTCCTCAGGCACCTCCTTC | |
| | ACCACCATTCCTACTCCTCCAGCGGCATCAGGATCGTCGAGCTGCCAT | |
| | TCAACTCCACCAACCACGGACTCCCACCGGGCATCGAGAACACCGATA | |
| | AGCTCACACTCCCGCTCGTGGTGTCCCTCTTCCATTCCACCATCAGCC | |
| | TCGATCCGCACCTCCGCGATTACATCTCCAGGCATTTCAGCCCAGCCA | |
| | GGCCACCACTCTGCGTGATCCATGATGTGTTCCTCGGCTGGGTTGACC | |
| | AGGTGGCCAAGGATGTGGGCTCTACAGGCGTGGTGTTCACAACAGGCG | |
| | GCGCTTATGGCACATCCGCCTACGTGTCCATCTGGAACGATCTCCCGC | |
| | ACCAGAACTACTCCGACGACCAAGAGTTCCCGCTGCCAGGCTTCCCAG | |
| | AGAACCATAAGTTCCGCAGGTCCCAGCTCCATCGGTTCCTCAGATATG | |
| | CCGACGGCTCCGACGATTGGTCCAAGTATTTCCAGCCGCAGCTCCGCC | |
| | AGTCCATGAAGTCTTTTGGCTGGCTCTGCAACTCCGTGGAAGAGATCG | |
| | AGACACTCGGCTTCTCCATCCTCCGCAACTACACCAAGCTGCCGATCT | |
| | GGGGCATCGGCCCACTTATTGCTTCCCCAGTGCAGCACTCCTCCTCCG | |
| | ACAACAATTCAACAGGCGCCGAGTTCGTGCAGTGGCTCAGCCTCAAAG | |
| | AGCCGGACTCCGTCCTCTACATCTCCTTCGGCTCCCAGAACACGATCA | |
| | GCCCGACGCAGATGATGGAACTCGCTGCTGGCCTTGAGTCCTCCGAGA | |
| | AGCCATTCCTCTGGGTGATCAGAGCCCCGTTCGGCTTCGACATCAACG | |
| | AAGAGATGCGCCCAGAGTGGCTGCCAGAGGGCTTTGAGGAACGCATGA | |
| | AGGTGAAGAAACAGGGCAAGCTCGTGTACAAGCTCGGCCCGCAGCTTG | |
| | AGATCCTCAACCATGAATCCATCGGCGGCTTTCTCACCCACTGCGGAT | |
| | GGAACAGCATCCTTGAGTCTCTTCGCGAGGGCGTTCCGATGCTTGGAT | |
| | GGCCACTTGCTGCCGAGCAGGCCTACAACCTCAAGTACCTCGAAGATG | |
| | AGATGGGCGTCGCGGTTGAGCTTGCTAGAGGCCTCGAAGGCGAGATCT | |
| | CCAAAGAGAAGGTCAAGCGCATCGTCGAGATGATCCTTGAGCGCAACG | |
| | AGGGCTCCAAAGGCTGGGAGATGAAGAATCGCGCCGTGGAAATGGGCA | |
| | AAAAGCTCAAGGACGCCGTGAACGAGGAAAAAGAGCTGAAGGGCTCCT | |
| | CCGTGAAGGCGATCGACGATTTCCTCGACGCCGTCATGCAGGCCAAAC | |
| | TTGAGCCAAGCCTCCAGTGA | |
| ZmUbi - | CTGCAGTGCAGCGTGACCCGGTCGTGCCCCTCTCTAGAGATAATGAGC | 157 |
| ADH - | ATTGCATGTCTAAGTTATAAAAAATTACCACATATTTTTTTTGTCACA | |
| PeaChISP - | CTTGTTTGAAGTGCAGTTTATCTATCTTTATACATATATTTAAACTTT | |
| AroG | ACTCTACGAATAATATAATCTATAGTACTACAATAATATCAGTGTTTT | |
| | AGAGAATCATATAAATGAACAGTTAGACATGGTCTAAAGGACAATTGA | |
| | GTATTTTGACAACAGGACTCTACAGTTTTATCTTTTTAGTGTGCATGT | |
| | GTTCTCCTTTTTTTTTGCAAATAGCTTCACCTATATAATACTTCATCC | |
| | ATTTTATTAGTACATCCATTTAGGGTTTAGGGTTAATGGTTTTTATAG | |
| | ACTAATTTTTTTAGTACATCTATTTTATTCTATTTTAGCCTCTAAATT | |
| | AAGAAAACTAAAACTCTATTTTAGTTTTTTTATTTAATAATTTAGATA | |
| | TAAAATAGAATAAAATAAAGTGACTAAAAATTAAACAAATACCCTTTA | |
| | AGAAATTAAAAAAACTAAGGAAACATTTTTCTTGTTTCGAGTAGATAA | |
| | TGCCAGCCTGTTAAACGCCGTCGACGAGTCTAACGGACACCAACCAGC | |
| | GAACCAGCAGCGTCGCGTCGGGCCAAGCGAAGCAGACGGCACGGCATC | |
| | TCTGTCGCTGCCTCTGGACCCCTCTCGAGAGTTCCGCTCCACCGTTGG | |
| | ACTTGCTCCGCTGTCGGCATCCAGAAATTGCGTGGCGGAGCGGCAGAC | |
| | GTGAGCCGGCACGGCAGGCGGCCTCCTCCTCCTCTCACGGCACCGGCA | |
| | GCTACGGGGGATTCCTTTCCCACCGCTCCTTCGCTTTCCCTTCCTCGC | |
| | CCGCCGTAATAAATAGACACCCCCTCCACACCCTCTTTCCCCAACCTC | |
| | GTGTTGTTCGGAGCGCACACACACACAACCAGATCTCCCCCAAATCCA | |
| | CCCGTCGGCACCTCCGCTTCAAGGTACGCCGCTCGTCCTCCCCCCCCC | |
| | CCCTCTCTACCTTCTCTAGATCGGCGTTCCGGTCCATGGTTAGGGCCC | |
| | GGTAGTTCTACTTCTGTTCATGTTTGTGTTAGATCCGTGTTTGTGTTA | |
| | GATCCGTGCTGCTAGCGTTCGTACACGGATGCGACCTGTACGTCAGAC | |
| | ACGTTCTGATTGCTAACTTGCCAGTGTTTCTCTTTGGGGAATCCTGGG | |
| | ATGGCTCTAGCCGTTCCGCAGACGGGATCGATTTCATGATTTTTTTTG | |
| | TTTCGTTGCATAGGGTTTGGTTTGCCCTTTTCCTTTATTTCAATATAT | |
| | GCCGTGCACTTGTTTGTCGGGTCATCTTTTCATGCTTTTTTTTGTCTT | |
| | GGTTGTGATGATGTGGTCTGGTTGGGCGGTCGTTCTAGATCGGAGTAG | |
| | AATTCTGTTTCAAACTACCTGGTGGATTTATTAATTTTGGATCTGTAT | |
| | GTGTGTGCCATACATATTCATAGTTACGAATTGAAGATGATGGATGGA | |
| | AATATCGATCTAGGATAGGTATACATGTTGATGCGGGTTTTACTGATG | |
| | CATATACAGAGATGCTTTTTGTTCGCTTGGTTGTGATGATGTGGTGTG | |
| | GTTGGGCGGTCGTTCATTCGTTCTAGATCGGAGTAGAATACTGTTTCA | |
| | AACTACCTGGTGTATTTATTAATTTTGGAACTGTATGTGTGTGTCATA | |
| | CATCTTCATAGTTACGAGTTTAAGATGGATGGAAATATCGATCTAGGA | |
| | TAGGTATACATGTTGATGTGGGTTTTACTGATGCATATACATGATGGC | |
| | ATATGCAGCATCTATTCATATGCTCTAACCTTGAGTACCTATCTATTA | |
| | TAATAAACAAGTATGTTTTATAATTATTTTGATCTTGATATACTTGGA | |
| | TGATGGCATATGCAGCAGCTATATGTGGATTTTTTTAGCCCTGCCTTC | |
| | ATACGCTATTTATTTGCTTGGTACTGTTTCTTTTGTCGATGCTCACCC | |
| | TGTTGTTTGGTGTTACTTCTGCAGAATGATATCACTATCCTCTTTTCA | |
| | TCCATCAAGCACAACTGCTACCGCTACCGCAGCTGCCGCAACCACACA | |
| | CCCTCCACAGCAATGCCCCGCATTCTCTTCACCACCTTCGCATCTATC | |
| | CTTGCCGCTACGACACCCTCGTCAACATCTTGTGGTTAGGTGTGGAGG | |
| | GGGTGGCTCTGCATCGGAGTCCGTATTCAACAGAGATTCAGCCGCAAC | |
| | TAGGGTTTCAAACGATCACTTGGATGTATCTAAAAGGGATGTAAAACT | |
| | CAAAATTGCTATCATAGGGTTCGGAAACTTTGGGCAATTTCTCGCAAA | |
| | GACTATGGCAAAACAAGGTCATAGAGTACTAGCATATTCTCGATCAGA | |
| | CTATTCCCGGGCGGCAAAAGAGATTGGAGTTGAATATTTTACTGATGC | |
| | AGATGACTTGTGTGAGGAACATCCTGAAGTAATTCTTTTGTGTACATC | |
| | TATACTGTCTACTGAGAAGGTGTTGCGGTCATTACCTCTCCATAGATT | |
| | GCGCAGATCAACCCTGTTCGCTGATGTTTTGAGTGTGAAAGAGTTCCC | |
| | TCGCTCTTTATTTCTGCAACTCTTGCCCAAAGATTTTGATATACTCTG | |
| | TACCCACCCTATGTTTGGGCCAGACAGCGGGAAAGACGGGTGGGGCGG | |
| | ACTCCCATTCGTATTTGACAAAGTAAGGGTGGGCAGCGACCAATCTCG | |
| | GACGTCAAGAGCTGAGGCATTTTTAGATGTTTTTAGAAACGCCGGGTG | |
| | TAGAATGGTGGAAATGTCATGCGTAGATCATGACAAACATGCCGCAGG | |
| | CTCACAATTTATTACACACATGATGGGAAGGGTACTGGAAAAACTAGC | |
| | ACTTGAAAATACGCCAATTAACACAAAGGGATACGAATCATTACTAAA | |
| | TCTAGTTGATAACACAGCACGCGACAGCTTTGAATTGTTTTACGGCCT | |
| | ATTCCTTTATAACAAGAATGCTATGGAGCAGTTGGATAGAATGGACTG | |
| | GGCTTTTGAGATGGTGAAAAAGCAGCTAAGCGGTTATTTGCATGATCT | |
| | GGTGAGGAAACAACTAATGCTGGAAGGGAACAATGATCAGGCGGAGGT | |
| | CACCTTCGATAAGCCCCTGATGCTACCCTCTCCCACAATTAACCCACC | |
| | TCAAATAGTGCCCTCCGCTGACATGGCTGAGAAAAAGCACGATCTTGT | |
| | AGTTGTCAATGGGACTAGGATGGCTTCTATGATATCCTCTTCAGCTGT | |
| | GACTACAGTCAGCCGTGCTTCTACGGTGCAATCGGCCGCGGTGGCTCC | |
| | ATTCGGCGGCCTCAAATCCATGACTGGATTCCCAGTTAAGAAGGTCAA | |
| | CACTGACATTACTTCCATTACAAGCAATGGTGGAAGAGTAAAGTGCAT | |
| | GCAGATGAATTACCAGAACGATGACTTAAGAATCAAAGAAATTAAAGA | |
| | GTTGTTACCACCTGTGGCTCTTTTGGAAAAATTCCCAGCAACTGAGAA | |
| | TGCTGCAAACACAGTTGCTCATGCAAGAAAGGCTATTCACAAAATCTT | |
| | GAAGGGTAATGATGACAGGTTACTTGTTGTGATCGGACCATGCTCAAT | |
| | ACATGATCCTGTTGCTGCAAAGGAATACGCTACTAGATTGCTTGCATT | |
| | GAGGGAAGAGTTAAAGGATGAACTTGAGATTGTTATGAGAGTGTACTT | |
| | CGAGAAACCAAGGACCACTGTTGGTTGGAAGGGACTTATCAATGATCC | |
| | TCACATGGACAACTCCTTCCAAATTAATGATGGTTTGAGAATCGCTAG | |
| | GAAACTTTTGCTTGATATTAACGACTCAGGTTTGCCAGCTGCAGGAGA | |
| | ATTTTTAGATATGATCACACCTCAGTACTTAGCTGACCTTATGTCATG | |
| | GGGTGCTATAGGAGCAAGAACAACCGAAAGTCAAGTTCATAGGGAGCA | |
| | GGCTTCCGGTTTGTCTTGTCCAGTGGGATTCAAAAATGGTACTGATGG | |
| | AACAATTAAGGTTGCTATAGACGCAATTAACGCTGCAGGTGCTCCTCA | |
| | TTGTTTTCTTTCTGTTACAAAATGGGGACACTCAGCAATCGTGAATAC | |
| | CAGTGGTAACGGAGATTGCCATATTATCTTGAGAGGTGGAAAAGAACC | |
| | AAATTATTCAGCTAAGCACGTTGCAGAAGTGAAAGAGGGTTTGAACAA | |
| | GGCTGGATTACCTGCACAAGTTATGATCGATTTCTCTCATGCTAACTC | |
| | CTCTAAGCAATTCAAGAAACAGATGGATGTTTGTGCTGACGTGTGCCA | |
| | ACAGATCGCTGGTGGAGAAAAGGCTATTATTGGTGTTATGGTGGAAAG | |
| | TCACTTAGTTGAGGGAAATCAATCATTAGAAAGTGGAGAGCCTCTTGC | |
| | TTACGGAAAATCTATTACCGATGCATGCATCGGTTGGGAAGATACTGA | |
| | CGCTCTTTTGAGACAGTTGGCTAACGCAGTTAAGGCAAGAAGGGGTTG | |
| | A | |
| 2x 35S + | GGAGGTCAACATGGTGGAGCACGACACTCTGGTCTACTCCAAAAATGT | 158 |
| TMV | CAAAGATACAGTCTCAGAAGATCAAAGGGCTATTGAGACTTTTCAACA | |
| 5′UTR - | AAGGATAATTTCGGGAAACCTCCTCGGATTCCATTGCCCAGCTATCTG | |
| CYP76AD1 - | TCACTTCATCGAAAGGACAGTAGAAAAGGAAGGTGGCTCCTACAAATG | |
| P2A - | CCATCATTGCGATAAAGGAAAGGCTATCATTCAAGATCTCTCTGCCGA | |
| DODA - | CAGTGGTCCCAAAGATGGACCCCCACCCACGAGGAGCATCGTGGAAAA | |
| P2A - | AGAAGAGGTTCCAACCACGTCTACAAAGCAAGTGGATTGATGTGATAA | |
| cDOPA5GT | CATGGTGGAGCACGACACTCTGGTCTACTCCAAAAATGTCAAAGATAC | |
| | AGTCTCAGAAGATCAAAGGGCTATTGAGACTTTTCAACAAAGGATAAT | |
| | TTCGGGAAACCTCCTCGGATTCCATTGCCCAGCTATCTGTCACTTCAT | |
| | CGAAAGGACAGTAGAAAAGGAAGGTGGCTCCTACAAATGCCATCATTG | |
| | CGATAAAGGAAAGGCTATCATTCAAGATCTCTCTGCCGACAGTGGTCC | |
| | CAAAGATGGACCCCCACCCACGAGGAGCATCGTGGAAAAAGAAGAGGT | |
| | TCCAACCACGTCTACAAAGCAAGTGGATTGATGTGACATCTCCACTGA | |
| | CGTAAGGGATGACGCACAATCCCACTATCCTTCGCAAGACCCTTCCTC | |
| | TATATAAGGAAGTTCATTTCATTTGGAGAGGACACGCTCGAGTATAAG | |
| | AGCTCATTTTTACAACAATTACCAACAACAACAAACAACAAACAACAT | |
| | TACAATTACATTTACAATTATCGATACAATGGATCATGCGACCCTCGC | |
| | CATGATCCTCGCGATCTGGTTCATCAGCTTCCACTTCATCAAGCTGCT | |
| | GTTCTCCCAGCAGACCACCAAGCTGCTTCCGCCAGGACCAAAGCCGCT | |
| | TCCGATCATCGGCAACATCCTTGAGGTGGGCAAGAAGCCGCATCGGTC | |
| | CTTCGCCAACCTCGCCAAGATTCACGGCCCACTCATTTCCCTCAGACT | |
| | CGGCTCTGTGACCACCATCGTTGTGTCCTCTGCCGACGTGGCCAAAGA | |
| | GATGTTCCTCAAGAAGGATCACCCGCTCTCCAACCGCACGATCCCGAA | |
| | TAGTGTTACAGCCGGCGACCACCACAAGCTCACCATGTCTTGGCTCCC | |
| | GGTGTCTCCGAAGTGGCGCAACTTCCGCAAGATTACCGCCGTGCATCT | |
| | GCTCTCCCCACAGAGACTCGATGCCTGCCAGACATTCAGGCACGCCAA | |
| | GGTGCAGCAGCTCTACGAGTACGTTCAAGAGTGCGCCCAGAAAGGCCA | |
| | GGCCGTGGATATTGGCAAGGCCGCCTTTACGACCAGCCTCAACCTCCT | |
| | CAGCAAGCTGTTCTTCAGCGTCGAGCTGGCGCACCACAAGTCCCATAC | |
| | CAGCCAAGAGTTCAAAGAGCTGATCTGGAACATCATGGAAGATATAGG | |
| | CAAGCCGAACTACGCCGACTACTTCCCGATTCTCGGCTGCGTTGACCC | |
| | ATCTGGCATTAGAAGAAGGCTCGCCTGCTCCTTCGACAAGCTGATCGC | |
| | CGTGTTCCAGGGCATCATCTGCGAGAGACTCGCCCCAGATTCCTCCAC | |
| | CACAACTACCACCACCACCGACGACGTGCTCGATGTGCTCCTCCAGCT | |
| | GTTCAAGCAGAACGAGCTGACGATGGGCGAGATCAACCACCTCCTCGT | |
| | GGACATCTTCGACGCCGGCACCGATACCACATCCTCCACATTCGAGTG | |
| | GGTGATGACCGAGCTGATCCGCAATCCAGAGATGATGGAAAAGGCCCA | |
| | AGAGGAAATCAAGCAGGTCCTCGGCAAGGACAAGCAGATCCAAGAGTC | |
| | CGACATCATCAACCTGCCGTACCTCCAGGCGATCATCAAAGAGACACT | |
| | CCGCCTCCATCCGCCGACCGTGTTCTTGCTCCCAAGAAAGGCCGACAC | |
| | CGATGTCGAGCTGTACGGCTACATCGTGCCGAAGGATGCCCAGATCCT | |
| | CGTGAACCTCTGGGCCATTGGCAGGGACCCAAACGCCTGGCAGAACGC | |
| | CGATATTTTCAGCCCAGAGCGCTTCATCGGCTGCGAGATCGATGTTAA | |
| | GGGCCGCGATTTCGGCCTCCTTCCATTTGGCGCTGGCCGCAGAATTTG | |
| | CCCAGGCATGAATCTCGCCATCAGGATGCTCACCCTCATGCTCGCCAC | |
| | ACTCCTCCAGTTCTTCAACTGGAAGCTCGAAGGCGACATCTCCCCGAA | |
| | GGACCTCGACATGGACGAGAAGTTCGGCATTGCGCTCCAAAAGACCAA | |
| | GCCGCTCAAGCTCATCCCGATTCCGCGCTACGGTAGCGGAGCTACCAA | |
| | TTTTAGCCTCCTTAAGCAGGCAGGTGATGTAGAAGAGAACCCCGGGCC | |
| | TATGAAGATGATGAACGGCGAGGACGCCAACGACCAGATGATCAAAGA | |
| | GTCCTTCTTCATCACCCACGGCAACCCGATCCTCACCGTCGAGGATAC | |
| | ACATCCGCTCAGGCCGTTCTTCGAGACATGGCGCGAGAAGATTTTCTC | |
| | CAAGAAGCCGAAGGCCATCCTCATCATCTCCGGCCACTGGGAGACAGT | |
| | GAAGCCAACCGTGAACGCCGTGCACATCAACGACACCATCCACGACTT | |
| | CGACGACTACCCAGCCGCCATGTACCAGTTCAAGTACCCAGCTCCAGG | |
| | CGAGCCAGAGCTTGCGAGAAAGGTGGAAGAGATCCTCAAGAAGTCCGG | |
| | GTTCGAGACAGCCGAGACAGACCAAAAGAGGGGCCTTGATCACGGCGC | |
| | CTGGGTTCCACTCATGCTCATGTATCCAGAGGCGGACATCCCGGTGTG | |
| | CCAGCTCTCAGTTCAGCCACATCTCGACGGCACCTACCACTACAATCT | |
| | CGGCAGAGCCCTCGCGCCGCTCAAGAATGATGGCGTGCTCATTATTGG | |
| | CTCCGGCAGCGCCACACATCCACTCGATGAGACACCGCACTACTTCGA | |
| | TGGTGTTGCCCCTTGGGCCGCTGCCTTCGATTCTTGGCTTAGGAAGGC | |
| | CCTCATCAACGGCCGCTTCGAGGAAGTGAACATCTACGAGAGCAAGGC | |
| | CCCGAACTGGAAGCTCGCCCATCCATTTCCAGAGCACTTCTACCCGCT | |
| | CCACGTTGTGCTCGGCGCTGCTGGTGAAAAGTGGAAGGCCGAGCTGAT | |
| | CCACTCCTCCTGGGATCATGGCACACTTTGCCACGGCTCCTACAAGTT | |
| | CACCTCCGCCGGATCCGGAGCAACCAACTTTAGCCTGCTCAAGCAAGC | |
| | AGGAGATGTTGAGGAAAATCCTGGCCCCATGACCGCCATCAAGATGAA | |
| | CACCAACGGCGAGGGCGAGACACAGCACATCCTCATGATCCCGTTCAT | |
| | GGCGCAGGGCCACCTCAGGCCATTTCTCGAACTCGCCATGTTCCTCTA | |
| | CAAGCGCTCCCACGTGATCATCACCCTGCTCACAACTCCGCTCAATGC | |
| | CGGCTTCCTCAGGCACCTCCTTCACCACCATTCCTACTCCTCCAGCGG | |
| | CATCAGGATCGTCGAGCTGCCATTCAACTCCACCAACCACGGACTCCC | |
| | ACCGGGCATCGAGAACACCGATAAGCTCACACTCCCGCTCGTGGTGTC | |
| | CCTCTTCCATTCCACCATCAGCCTCGATCCGCACCTCCGCGATTACAT | |
| | CTCCAGGCATTTCAGCCCAGCCAGGCCACCACTCTGCGTGATCCATGA | |
| | TGTGTTCCTCGGCTGGGTTGACCAGGTGGCCAAGGATGTGGGCTCTAC | |
| | AGGCGTGGTGTTCACAACAGGCGGCGCTTATGGCACATCCGCCTACGT | |
| | GTCCATCTGGAACGATCTCCCGCACCAGAACTACTCCGACGACCAAGA | |
| | GTTCCCGCTGCCAGGCTTCCCAGAGAACCATAAGTTCCGCAGGTCCCA | |
| | GCTCCATCGGTTCCTCAGATATGCCGACGGCTCCGACGATTGGTCCAA | |
| | GTATTTCCAGCCGCAGCTCCGCCAGTCCATGAAGTCTTTTGGCTGGCT | |
| | CTGCAACTCCGTGGAAGAGATCGAGACACTCGGCTTCTCCATCCTCCG | |
| | CAACTACACCAAGCTGCCGATCTGGGGCATCGGCCCACTTATTGCTTC | |
| | CCCAGTGCAGCACTCCTCCTCCGACAACAATTCAACAGGCGCCGAGTT | |
| | CGTGCAGTGGCTCAGCCTCAAAGAGCCGGACTCCGTCCTCTACATCTC | |
| | CTTCGGCTCCCAGAACACGATCAGCCCGACGCAGATGATGGAACTCGC | |
| | TGCTGGCCTTGAGTCCTCCGAGAAGCCATTCCTCTGGGTGATCAGAGC | |
| | CCCGTTCGGCTTCGACATCAACGAAGAGATGCGCCCAGAGTGGCTGCC | |
| | AGAGGGCTTTGAGGAACGCATGAAGGTGAAGAAACAGGGCAAGCTCGT | |
| | GTACAAGCTCGGCCCGCAGCTTGAGATCCTCAACCATGAATCCATCGG | |
| | CGGCTTTCTCACCCACTGCGGATGGAACAGCATCCTTGAGTCTCTTCG | |
| | CGAGGGCGTTCCGATGCTTGGATGGCCACTTGCTGCCGAGCAGGCCTA | |
| | CAACCTCAAGTACCTCGAAGATGAGATGGGCGTCGCGGTTGAGCTTGC | |
| | TAGAGGCCTCGAAGGCGAGATCTCCAAAGAGAAGGTCAAGCGCATCGT | |
| | CGAGATGATCCTTGAGCGCAACGAGGGCTCCAAAGGCTGGGAGATGAA | |
| | GAATCGCGCCGTGGAAATGGGCAAAAAGCTCAAGGACGCCGTGAACGA | |
| | GGAAAAAGAGCTGAAGGGCTCCTCCGTGAAGGCGATCGACGATTTCCT | |
| | CGACGCCGTCATGCAGGCCAAACTTGAGCCAAGCCTCCAGTGA | |
| CsVMV - | CCAGAAGGTAATTATCCAAGATGTAGCATCAAGAATCCAATGTTTACG | 159 |
| ADH - | GGAAAAACTATGGAAGTATTATGTAAGCTCAGCAAGAAGCAGATCAAT | |
| PeaChISP - | ATGCGGCACATATGCAACCTATGTTCAAAAATGAAGAATGTACAGATA | |
| AroG | CAAGATCCTATACTGCCAGAATACGAAGAAGAATACGTAGAAATTGAA | |
| | AAAGAAGAACCAGGCGAAGAAAAGAATCTTGATGACGTAAGCACTGAC | |
| | GACAACAATGAAAAGAAGAAGATAAGGTCGGTGATTGTGAAAGAGACA | |
| | TAGAGGACACATGTAAGGTGGAAAATGTAAGGGCGGAAAGTAACCTTA | |
| | TCACAAAGGAATCTTATCCCCCACTACTTATCCTTTTATATTTTTCCG | |
| | TGTCATTTTTGCCCTTGAGTTTTCCTATATAAGGAACCAAGTTCGGCA | |
| | TTTGTGAAAACAAGAAAAAATTTGGTGTAAGCTATTTTCTTTGAAGTA | |
| | CTGAGGATACAACTTCAGAGAAATTTGTAAGTTTGTAATGATATCACT | |
| | ATCCTCTTTTCATCCATCAAGCACAACTGCTACCGCTACCGCAGCTGC | |
| | CGCAACCACACACCCTCCACAGCAATGCCCCGCATTCTCTTCACCACC | |
| | TTCGCATCTATCCTTGCCGCTACGACACCCTCGTCAACATCTTGTGGT | |
| | TAGGTGTGGAGGGGGTGGCTCTGCATCGGAGTCCGTATTCAACAGAGA | |
| | TTCAGCCGCAACTAGGGTTTCAAACGATCACTTGGATGTATCTAAAAG | |
| | GGATGTAAAACTCAAAATTGCTATCATAGGGTTCGGAAACTTTGGGCA | |
| | ATTTCTCGCAAAGACTATGGCAAAACAAGGTCATAGAGTACTAGCATA | |
| | TTCTCGATCAGACTATTCCCGGGCGGCAAAAGAGATTGGAGTTGAATA | |
| | TTTTACTGATGCAGATGACTTGTGTGAGGAACATCCTGAAGTAATTCT | |
| | TTTGTGTACATCTATACTGTCTACTGAGAAGGTGTTGCGGTCATTACC | |
| | TCTCCATAGATTGCGCAGATCAACCCTGTTCGCTGATGTTTTGAGTGT | |
| | GAAAGAGTTCCCTCGCTCTTTATTTCTGCAACTCTTGCCCAAAGATTT | |
| | TGATATACTCTGTACCCACCCTATGTTTGGGCCAGACAGCGGGAAAGA | |
| | CGGGTGGGGCGGACTCCCATTCGTATTTGACAAAGTAAGGGTGGGCAG | |
| | CGACCAATCTCGGACGTCAAGAGCTGAGGCATTTTTAGATGTTTTTAG | |
| | AAACGCCGGGTGTAGAATGGTGGAAATGTCATGCGTAGATCATGACAA | |
| | ACATGCCGCAGGCTCACAATTTATTACACACATGATGGGAAGGGTACT | |
| | GGAAAAACTAGCACTTGAAAATACGCCAATTAACACAAAGGGATACGA | |
| | ATCATTACTAAATCTAGTTGATAACACAGCACGCGACAGCTTTGAATT | |
| | GTTTTACGGCCTATTCCTTTATAACAAGAATGCTATGGAGCAGTTGGA | |
| | TAGAATGGACTGGGCTTTTGAGATGGTGAAAAAGCAGCTAAGCGGTTA | |
| | TTTGCATGATCTGGTGAGGAAACAACTAATGCTGGAAGGGAACAATGA | |
| | TCAGGCGGAGGTCACCTTCGATAAGCCCCTGATGCTACCCTCTCCCAC | |
| | AATTAACCCACCTCAAATAGTGCCCTCCGCTGACATGGCTGAGAAAAA | |
| | GCACGATCTTGTAGTTGTCAATGGGACTAGGATGGCTTCTATGATATC | |
| | CTCTTCAGCTGTGACTACAGTCAGCCGTGCTTCTACGGTGCAATCGGC | |
| | CGCGGTGGCTCCATTCGGCGGCCTCAAATCCATGACTGGATTCCCAGT | |
| | TAAGAAGGTCAACACTGACATTACTTCCATTACAAGCAATGGTGGAAG | |
| | AGTAAAGTGCATGCAGATGAATTACCAGAACGATGACTTAAGAATCAA | |
| | AGAAATTAAAGAGTTGTTACCACCTGTGGCTCTTTTGGAAAAATTCCC | |
| | AGCAACTGAGAATGCTGCAAACACAGTTGCTCATGCAAGAAAGGCTAT | |
| | TCACAAAATCTTGAAGGGTAATGATGACAGGTTACTTGTTGTGATCGG | |
| | ACCATGCTCAATACATGATCCTGTTGCTGCAAAGGAATACGCTACTAG | |
| | ATTGCTTGCATTGAGGGAAGAGTTAAAGGATGAACTTGAGATTGTTAT | |
| | GAGAGTGTACTTCGAGAAACCAAGGACCACTGTTGGTTGGAAGGGACT | |
| | TATCAATGATCCTCACATGGACAACTCCTTCCAAATTAATGATGGTTT | |
| | GAGAATCGCTAGGAAACTTTTGCTTGATATTAACGACTCAGGTTTGCC | |
| | AGCTGCAGGAGAATTTTTAGATATGATCACACCTCAGTACTTAGCTGA | |
| | CCTTATGTCATGGGGTGCTATAGGAGCAAGAACAACCGAAAGTCAAGT | |
| | TCATAGGGAGCAGGCTTCCGGTTTGTCTTGTCCAGTGGGATTCAAAAA | |
| | TGGTACTGATGGAACAATTAAGGTTGCTATAGACGCAATTAACGCTGC | |
| | AGGTGCTCCTCATTGTTTTCTTTCTGTTACAAAATGGGGACACTCAGC | |
| | AATCGTGAATACCAGTGGTAACGGAGATTGCCATATTATCTTGAGAGG | |
| | TGGAAAAGAACCAAATTATTCAGCTAAGCACGTTGCAGAAGTGAAAGA | |
| | GGGTTTGAACAAGGCTGGATTACCTGCACAAGTTATGATCGATTTCTC | |
| | TCATGCTAACTCCTCTAAGCAATTCAAGAAACAGATGGATGTTTGTGC | |
| | TGACGTGTGCCAACAGATCGCTGGTGGAGAAAAGGCTATTATTGGTGT | |
| | TATGGTGGAAAGTCACTTAGTTGAGGGAAATCAATCATTAGAAAGTGG | |
| | AGAGCCTCTTGCTTACGGAAAATCTATTACCGATGCATGCATCGGTTG | |
| | GGAAGATACTGACGCTCTTTTGAGACAGTTGGCTAACGCAGTTAAGGC | |
| | AAGAAGGGGTTGA | |

### Constructs

Among other things, the present disclosure provides that some polynucleotides as described herein are constructs (e.g., polynucleotide constructs). In some embodiments, constructs (e.g., polynucleotide constructs) comprising a coding sequence and may also be referred to herein as a polynucleotide vectors. In some embodiments, polynucleotide vectors suitable for engineering of CPOS (e.g., plants) and/or microbes are described herein. In some embodiments, polynucleotide vectors suitable for transgene introduction into certain CPOS (e.g., plants) and/or microbes are described herein.

In some embodiments, a coding sequence may comprise the genetic information required to create useful products, e.g., RNA and/or polypeptides that may confer desirable traits (e.g., expression/production of a relevant pigment and/or of a component of a betalain metabolic pathway).

In some embodiments, a vector described herein can further include regulatory and/or control sequences that alter the transcription and/or translation of an encoded gene, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of transcriptional and/or translational control sequences are described herein.

In some embodiments, technologies described herein comprise a vector. In some embodiments, a vector is a transgenic vector. In some embodiments, a transgenic vector comprises a cloning vector. In some embodiments, a transgenic vector comprises an engineered polynucleotide suitable for introduction into an organism.

In some embodiments, a transgenic vector may comprise a backbone sequence. In some embodiments, a transgenic vector may comprise at least one promoter. In some embodiments, a transgenic vector may comprise at least one 5′ UTR. In some embodiments, a transgenic vector may comprise at least one organelle localization signal. In some embodiments, a transgenic vector may comprise at least one gene of interest (e.g., an enzyme and/or polypeptide of interest). In some embodiments, a transgenic vector may comprise at least one tag sequence (e.g., a fluorescent tag). In some embodiments, a transgenic vector may comprise at least one 3′ UTR. In some embodiments, a transgenic vector may comprise at least one transcription termination sequence. In some embodiments, a transgenic vector may comprise at least one selectable marker.

In some embodiments, the present disclosure provides compositions and methods suitable for engineering polynucleotide vectors (e.g., plasmids etc.). In some embodiments, a polynucleotide vector comprises at least one transgene to be inserted into a plant and/or microbes genome (e.g., any store of genetic information, e.g., nuclear DNA, mitochondrial DNA, chloroplastic DNA etc.). One skilled in the art will recognize that in some embodiments, many molecular biology methodologies now exist that may facilitate engineering of vectors suitable for transgenic engineering. For example, in some embodiments, a method suitable for transgenic engineering may comprise the use of golden gate DNA assembly systems. In some embodiments, golden gate DNA assembly systems may be particularly amenable for creation of compositions described herein. In some embodiments, a transgenic engineering system comprises a three step hierarchical modular cloning scheme. In some embodiments, a golden gate DNA assembly system facilitates high efficiency assembly of complex multigene vectors that can encode entire pathways. In some embodiments, multigene vectors may begin as libraries of basic modules containing regulatory and/or coding sequences. In some embodiments, a cloning process utilizes type IIS restriction enzymes. In some embodiments, transgenic engineering (e.g., for metabolic engineering) can be rendered highly efficient through use of golden gate DNA assembly systems as the inherent modularity facilitates iterative design, and building of multiple variants of a particular genetic circuit. In some embodiments, expression ratios of several genes can be obtained, and optimal parameters for a synthetic pathway can be engineered and tested in parallel. In some embodiments, use of restriction enzymes during golden gate DNA assembly allows for high throughput engineering. In some embodiments, use of restriction enzymes during golden gate DNA assembly allows for error-free engineering. In some embodiments, use of restriction enzymes during golden gate DNA assembly allows for both high throughput and error-free engineering, which can be considered highly advantageous over traditional PCR-based cloning techniques. One skilled in the art will recognize that multiple DNA assembly and/or cloning technologies exist and may be suitable for the creation of vectors, and/or compositions described herein.

In some embodiments, metabolic pathways described herein (e.g., pathways suitable for transgenic engineering, e.g., metabolic engineering) are tested in parallel, e.g., by simultaneously launching transformation of dozens of plant lines each with at least one DNA vector. In some embodiments, metabolic pathways described herein (e.g., pathways suitable for transgenic engineering, e.g., metabolic engineering) are tested in parallel, e.g., by simultaneously launching the transformation of dozens of plant lines each with at least one different DNA vector. In some embodiments, compositions and methods describe herein are tested using a protoplasts system (e.g., a cell suspension). In some embodiments, use of golden gate DNA assembly and/or protoplast systems permits in vivo testing prior to plant transformation.

In some embodiments, a vector for metabolic engineering as described herein can be or comprise but is not limited to, a plasmid, a transposon, a cosmid, an artificial chromosome (e.g., a human artificial chromosome (HAC), a yeast artificial chromosome (YAC), a bacterial artificial chromosome (BAC), a P1-derived artificial chromosome (PAC)), a viral vector, a Gateway® plasmid, etc. In some embodiments, suitable vectors provided herein can be of different sizes.

In some embodiments, a vector is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, up to about 15 kb, up to about 16 kb, up to about 17 kb, up to about 18 kb, up to about 19 kb, up to about 20 kb, up to about 21 kb, up to about 22 kb, up to about 23 kb, up to about 24 kb, up to about 25 kb, up to about 26 kb, up to about 27 kb, up to about 28 kb, up to about 29 kb, up to about 30 kb, up to about 31 kb, up to about 32 kb, up to about 33 kb, up to about 34 kb, or up to about 35 kb. In some embodiments, a vector is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, about 1 kb to about 15 kb, 1 kb to about 16 kb, about 1 kb to about 17 kb, about 1 kb to about 18 kb, about 1 kb to about 19 kb, about 1 kb to about 20 kb, about 1 kb to about 21 kb, about 1 kb to about 22 kb, about 1 kb to about 23 kb, about 1 kb to about 24 kb, about 1 kb to about 25 kb, about 1 kb to about 26 kb, about 1 kb to about 27 kb, about 1 kb to about 28 kb, about 1 kb to about 29 kb, about 1 kb to about 30 kb, about 2 kb to about 12 kb, about 2 kb to about 14 kb, about 2 kb to about 16 kb, about 2 kb to about 18 kb, about 2 kb to about 20 kb, about 2 kb to about 22 kb, about 2 kb to about 24 kb, about 2 kb to about 26 kb, about 2 kb to about 28 kb, about 2 kb to about 30 kb, about 5 kb to about 10 kb, about 5 kb to about 12 kb, about 5 kb to about 14 kb, about 5 kb to about 16 kb, about 5 kb to about 18 kb, about 5 kb to about 20 kb, about 5 kb to about 22 kb, about 5 kb to about 24 kb, about 5 kb to about 26 kb, about 5 kb to about 28 kb, about 5 kb to about 30 kb, about 5 kb to about 32 kb, about 5 kb to about 34 kb, about 5 kb to about 36 kb, about 10 kb to about 12 kb, about 10 kb to about 14 kb, about 10 kb to about 16 kb, about 10 kb to about 18 kb, about 10 kb to about 20 kb, about 10 kb to about 22 kb, about 10 kb to about 24 kb, about 10 kb to about 26 kb, about 10 kb to about 28 kb, about 10 kb to about 30 kb, about 14 kb to about 16 kb, about 14 kb to about 18 kb, about 14 kb to about 20 kb, about 14 kb to about 22 kb, about 14 kb to about 24 kb, about 14 kb to about 26 kb, about 14 kb to about 28 kb, about 14 kb to about 30 kb, about 18 kb to about 20 kb, about 18 kb to about 22 kb, about 18 kb to about 24 kb, about 18 kb to about 26 kb, about 18 kb to about 28 kb, about 14 kb to about 30 kb, about 14 kb to about 32 kb, about 16 kb to about 34 kb, about 18 kb to about 36 kb, about 20 kb to about 22 kb, about 20 kb to about 24 kb, about 20 kb to about 26 kb, about 20 kb to about 28 kb, about 20 kb to about 30 kb, about 20 kb to about 32 kb, about 20 kb to about 34 kb, about 20 kb to about 36 kb, about 26 kb to about 30 kb, about 28 kb to about 30 kb, about 24 to about 26 kb, or about 25 to about 27 kb.

In some embodiments, a vector is an artificial chromosome and can include a total length of up to about 3000 kb, up to about 2900 kb, up to about 2800 kb, up to about 2700 kb, up to about 2600 kb, up to about 2500 kb, up to about 2400 kb, up to about 2300 kb, up to about 2200 kb, up to about 2100 kb, up to about 2000 kb, up to about 1900 kb, up to about 1800 kb, up to about 1700 kb, up to about 1600 kb, up to about 1500 kb, up to about 1400 kb, up to about 1300 kb, up to about 1200 kb, up to about 1100 kb, up to about 1000 kb, up to about 900 kb, up to about 800 kb, up to about 700 kb, up to about 600 kb, up to about 500 kb, up to about 400 kb, up to about 375 kb, up to about 350 kb, up to about 325 kb, up to about 300 kb, up to about 275 kb, up to about 250 kb, up to about 225 kb, up to about 200 kb, up to about 175 kb, up to about 150 kb, or up to about 125 kb.

In some embodiments, a vector is a viral vector and can have a total number of nucleotides of up to 10 kb. In some embodiments, a viral vector can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, about 1 kb to about 15 kb, about 1 kb to about 16 kb, about 1 kb to about 17 kb, about 1 kb to about 18 kb, about 1 kb to about 19 kb, about 1 kb to about 20 kb, about 1 kb to about 21 kb, about 1 kb to about 22 kb, about 1 kb to about 23 kb, about 1 kb to about 24 kb, about 1 kb to about 25 kb, about 1 kb to about 26 kb, about 1 kb to about 27 kb, about 1 kb to about 28 kb, about 1 kb to about 29 kb, or about 1 kb to about 30 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 2 kb to about 12 kb, about 2 kb to about 14 kb, about 2 kb to about 16 kb, about 2 kb to about 18 kb, about 2 kb to about 20 kb, about 2 kb to about 22 kb, about 2 kb to about 24 kb, about 2 kb to about 26 kb, about 2 kb to about 28 kb, about 2 kb to about 30 kb, about 5 kb to about 10 kb, about 5 kb to about 12 kb, about 5 kb to about 14 kb, about 5 kb to about 16 kb, about 5 kb to about 18 kb, about 5 kb to about 20 kb, about 5 kb to about 22 kb, about 5 kb to about 24 kb, about 5 kb to about 26 kb, about 5 kb to about 28 kb, about 5 kb to about 30 kb, about 10 kb to about 12 kb, about 10 kb to about 14 kb, about 10 kb to about 16 kb, about 10 kb to about 18 kb, about 10 kb to about 20 kb, about 10 kb to about 22 kb, about 10 kb to about 24 kb, about 10 kb to about 26 kb, about 10 kb to about 28 kb, about 10 kb to about 30 kb, about 14 kb to about 16 kb, about 14 kb to about 18 kb, about 14 kb to about 20 kb, about 14 kb to about 22 kb, about 14 kb to about 24 kb, about 14 kb to about 26 kb, about 14 kb to about 28 kb, about 14 kb to about 30 kb, about 18 kb to about 20 kb, about 18 kb to about 22 kb, about 18 kb to about 24 kb, about 18 kb to about 26 kb, about 18 kb to about 28 kb, about 14 kb to about 30 kb, about 20 kb to about 22 kb, about 20 kb to about 24 kb, about 26 kb to about 30 kb, about 28 kb to about 30 kb, or about 24 to about 26 kb.

### Exemplary Polynucleotide Constructs

In some embodiments, a polynucleotide construct described herein is at least 85%, at least 90%, at least 91%, at least 92%, at least 93%, at least 94%, at least 95%, at least 96%, at least 97%, at least 98%, or at least 99% identical to a sequence included in Table 13. In some embodiments, a polynucleotide construct described herein has a polynucleotide sequence provided in Table 13.

TABLE 13
Exemplary Construct Polynucleotide Sequences

| | | SEQ ID |
|---|---|---|
| Description | Sequence | NO: |
| PL-888 | GGAGGAAGCCAACTAAACAAGACCATAACCATGGTGACATTTGACATA | 160 |
| construct | GTTGTTTACTACTTGCTTGAGCCCCACCCTTGCTTATCGGTTGAACAT | |
| | TACAAGATACACTGCGGGTGGCCTAAGGCACACCGTCCGAAACCGGCA | |
| | AACCAAGCCTGATCGCCGAAATCCAAAATCACTACCGGCAATCTCTAA | |
| | AGTTTATTTCATCCTTATATGACGAGGAAAGAAAAGAAGAGAGAAATA | |
| | ATATCTTAACTTCTAAATCAGTCGCGTCAACTTTCTCGGCTAAGAAAG | |
| | TGAGCACTATCATTTCGCAGACCATGTCATGAGTGCCGACTTGCCATA | |
| | TCTTATTATATTCTTATTTATTTAATTATAATCCCATTGCAATACGTC | |
| | TATTCTATCATGGCCTGCCACTAACGCTCCGTCTAACGTCGTTAAGCC | |
| | ATTGTCATAAGCGGCTGCTCAAAACTCTTCCCGGTGGAGGCGAGGCGT | |
| | TAACGGCGTCTACAAATCTAACGGCCACCAACCATCCAGCCGCCTCTC | |
| | GAAAGCTCCGCTCCGATCGCGGAAATTGCGTGGCGGAGACGAGCGGGC | |
| | TCCTCTCACACGGCCCGGAACCGTCACGGCACGGGTGGGGGATTCCTT | |
| | CCCCAACCCTCCCCACCTCTCCTCCCCCCGTCGCAGCCCATAAATACA | |
| | GGGCCCTCCGCGCCTCTTCCCACAATCTCACATCGTCTCATCGTTCGG | |
| | AGCGCACAACCCCCGGGTTCCAAATCCAAATTGCTCTTCTCGCGACCC | |
| | TCGGCGATCCTTCCCCCGCTTCAAGGTACGGCGATCGTCTCCCCCGTC | |
| | CTCTTGCCCCATCTCCTCGCTCGGCGTGGTTTGGTGGTTCTGCTTGGT | |
| | CTGTGGCTAGGAACTAGGCTGAGGCGTTGACGAAATCATGCTAGATCC | |
| | GCGTGTTTCCTGATCGTGGGTGGCTGGGAGGTGGGGTTTTCGTGTAGA | |
| | TCTGATCGGTTCCGCTGTTTATCCTGTCATGCTCATGTGATTTGTGGG | |
| | GATTTTAGGTCGTTTGTCCGGGAATCGTGGGGTTGCTTCTAGGCTGTT | |
| | CGTAGATGAGATCGTTCTCACGATCTGCTGGGTCGCTGCCTAGGTTCA | |
| | GCTAGGTCTGCCCTGTTTTTGGGTTCGTTTTCGGGATCTGTACGTGCA | |
| | TCTATTATCTGGTTCGATGGTGCTAGCTAGGAACAAACAACTGATTCG | |
| | TCCGATCGATTGTTTTGTTGCCATGTGCAAGGTTAGGTCGTTATCTGA | |
| | TTGCTGTAGATCAGAGTAGAATAAGATCATCACAAGCTAGCTCTTGGG | |
| | CTTATTATGAATCTGCGTTTGTTGCATGATTAAGATGATTATGCTTTT | |
| | TCTTATGCTGCCGTTTGTATATGATGCGGTAGCTTTTAACTGAATAGC | |
| | ACACCTTTCCTGTTTAGTTAGATTAGATTAGATTGCATGATAGATGAG | |
| | GATATATGCTGCTACATCAGTTTGATGATTCTCTGGTACCTCATAATC | |
| | AACTAGCTCATGTGCTTAAATTGAAACTGCATGTGCCACATGATTAAG | |
| | ATGCTAAGATTGGTGAAGATATATACGCTGCTGTTCCTATAGGATCCT | |
| | GTAGCTTTTACCTGGTCAACATGCATCGTCCTGTTATGGATAGATATG | |
| | CATGATAGATGAAGATATGTACTGCTACAATTTGATGATTCTTTTGTG | |
| | CACCTGATGATCATGCATGCTCTTTGCCCTTACTTTGATATACTTGGA | |
| | TGATGGCATGCTTAGTACTAATGATGTGATGAACACACATGACCTGTT | |
| | GGTATGAATATGATGTTGCTGTTTGCTTGTGATGAGTTCTGTTTGTTT | |
| | ACTGCTAGGCACTTACCCTGTTGTCTGGTTCTCTTTTGCAGAATGGTA | |
| | GAACAAGACGGGCTGCATGCTGGATCACCTGCGGCCTGGGTCGAAAGG | |
| | CTATTCGGTTATGACTGGGCGCAGCAAACCATTGGGTGCAGCGATGCA | |
| | GCCGTCTTTCGACTCAGCGCCCAAGGTAGACCCGTATTGTTTGTGAAA | |
| | ACTGACTTATCCGGGGCACTGAATGAATTACAAGACGAAGCAGCCAGA | |
| | CTTAGTTGGTTAGCGACCACAGGTGTGCCCTGTGCAGCGGTCCTTGAC | |
| | GTAGTTACCGAGGCAGGACGGGACTGGTTATTGCTTGGAGAAGTACCG | |
| | GGCCAAGACCTGTTATCTAGCCACCTCGCCCCGGCTGAAAAAGTCTCA | |
| | ATTATGGCGGATGCTATGAGGCGACTTCATACATTGGACCCAGCCACG | |
| | TGTCCCTTTGATCATCAGGCGAAACACAGAATAGAACGTGCAAGGACA | |
| | CGAATGGAGGCTGGGCTCGTTGATCAAGACGATTTAGATGAAGAGCAT | |
| | CAAGGCTTGGCACCTGCTGAGTTATTTGCTCGGTTAAAAGCTAGAATG | |
| | CCAGACGGGGAAGATCTTGTCGTAACCCATGGCGATGCCTGCTTGCCA | |
| | AATATTATGGTGGAGAATGGTCGTTTCTCAGGGTTTATCGATTGTGGG | |
| | CGACTTGGCGTGGCCGACAGGTATCAAGATATAGCCCTAGCAACTAGA | |
| | GACATAGCCGAAGAGTTGGGTGGGGAGTGGGCTGACAGATTCCTCGTG | |
| | CTATACGGGATTGCCGCTCCCGATTCCCAGAGAATCGCATTTTATCGA | |
| | CTCTTAGACGAATTCTTTTAGGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTGCAAGAA | |
| | TTCAAGCTTGGAGCTGCAGTGCAGCGTGACCCGGTCGTGCCCCTCTCT | |
| | AGAGATAATGAGCATTGCATGTCTAAGTTATAAAAAATTACCACATAT | |
| | TTTTTTTGTCACACTTGTTTGAAGTGCAGTTTATCTATCTTTATACAT | |
| | ATATTTAAACTTTACTCTACGAATAATATAATCTATAGTACTACAATA | |
| | ATATCAGTGTTTTAGAGAATCATATAAATGAACAGTTAGACATGGTCT | |
| | AAAGGACAATTGAGTATTTTGACAACAGGACTCTACAGTTTTATCTTT | |
| | TTAGTGTGCATGTGTTCTCCTTTTTTTTTGCAAATAGCTTCACCTATA | |
| | TAATACTTCATCCATTTTATTAGTACATCCATTTAGGGTTTAGGGTTA | |
| | ATGGTTTTTATAGACTAATTTTTTTAGTACATCTATTTTATTCTATTT | |
| | TAGCCTCTAAATTAAGAAAACTAAAACTCTATTTTAGTTTTTTTATTT | |
| | AATAATTTAGATATAAAATAGAATAAAATAAAGTGACTAAAAATTAAA | |
| | CAAATACCCTTTAAGAAATTAAAAAAACTAAGGAAACATTTTTCTTGT | |
| | TTCGAGTAGATAATGCCAGCCTGTTAAACGCCGTCGACGAGTCTAACG | |
| | GACACCAACCAGCGAACCAGCAGCGTCGCGTCGGGCCAAGCGAAGCAG | |
| | ACGGCACGGCATCTCTGTCGCTGCCTCTGGACCCCTCTCGAGAGTTCC | |
| | GCTCCACCGTTGGACTTGCTCCGCTGTCGGCATCCAGAAATTGCGTGG | |
| | CGGAGCGGCAGACGTGAGCCGGCACGGCAGGCGGCCTCCTCCTCCTCT | |
| | CACGGCACCGGCAGCTACGGGGGATTCCTTTCCCACCGCTCCTTCGCT | |
| | TTCCCTTCCTCGCCCGCCGTAATAAATAGACACCCCCTCCACACCCTC | |
| | TTTCCCCAACCTCGTGTTGTTCGGAGCGCACACACACACAACCAGATC | |
| | TCCCCCAAATCCACCCGTCGGCACCTCCGCTTCAAGGTACGCCGCTCG | |
| | TCCTCCCCCCCCCCCCTCTCTACCTTCTCTAGATCGGCGTTCCGGTCC | |
| | ATGGTTAGGGCCCGGTAGTTCTACTTCTGTTCATGTTTGTGTTAGATC | |
| | CGTGTTTGTGTTAGATCCGTGCTGCTAGCGTTCGTACACGGATGCGAC | |
| | CTGTACGTCAGACACGTTCTGATTGCTAACTTGCCAGTGTTTCTCTTT | |
| | GGGGAATCCTGGGATGGCTCTAGCCGTTCCGCAGACGGGATCGATTTC | |
| | ATGATTTTTTTTGTTTCGTTGCATAGGGTTTGGTTTGCCCTTTTCCTT | |
| | TATTTCAATATATGCCGTGCACTTGTTTGTCGGGTCATCTTTTCATGC | |
| | TTTTTTTTGTCTTGGTTGTGATGATGTGGTCTGGTTGGGCGGTCGTTC | |
| | TAGATCGGAGTAGAATTCTGTTTCAAACTACCTGGTGGATTTATTAAT | |
| | TTTGGATCTGTATGTGTGTGCCATACATATTCATAGTTACGAATTGAA | |
| | GATGATGGATGGAAATATCGATCTAGGATAGGTATACATGTTGATGCG | |
| | GGTTTTACTGATGCATATACAGAGATGCTTTTTGTTCGCTTGGTTGTG | |
| | ATGATGTGGTGTGGTTGGGCGGTCGTTCATTCGTTCTAGATCGGAGTA | |
| | GAATACTGTTTCAAACTACCTGGTGTATTTATTAATTTTGGAACTGTA | |
| | TGTGTGTGTCATACATCTTCATAGTTACGAGTTTAAGATGGATGGAAA | |
| | TATCGATCTAGGATAGGTATACATGTTGATGTGGGTTTTACTGATGCA | |
| | TATACATGATGGCATATGCAGCATCTATTCATATGCTCTAACCTTGAG | |
| | TACCTATCTATTATAATAAACAAGTATGTTTTATAATTATTTTGATCT | |
| | TGATATACTTGGATGATGGCATATGCAGCAGCTATATGTGGATTTTTT | |
| | TAGCCCTGCCTTCATACGCTATTTATTTGCTTGGTACTGTTTCTTTTG | |
| | TCGATGCTCACCCTGTTGTTTGGTGTTACTTCTGCAGAATGGATCATG | |
| | CGACCCTCGCCATGATCCTCGCGATCTGGTTCATCAGCTTCCACTTCA | |
| | TCAAGCTGCTGTTCTCCCAGCAGACCACCAAGCTGCTTCCGCCAGGAC | |
| | CAAAGCCGCTTCCGATCATCGGCAACATCCTTGAGGTGGGCAAGAAGC | |
| | CGCATCGGTCCTTCGCCAACCTCGCCAAGATTCACGGCCCACTCATTT | |
| | CCCTCAGACTCGGCTCTGTGACCACCATCGTTGTGTCCTCTGCCGACG | |
| | TGGCCAAAGAGATGTTCCTCAAGAAGGATCACCCGCTCTCCAACCGCA | |
| | CGATCCCGAATAGTGTTACAGCCGGCGACCACCACAAGCTCACCATGT | |
| | CTTGGCTCCCGGTGTCTCCGAAGTGGCGCAACTTCCGCAAGATTACCG | |
| | CCGTGCATCTGCTCTCCCCACAGAGACTCGATGCCTGCCAGACATTCA | |
| | GGCACGCCAAGGTGCAGCAGCTCTACGAGTACGTTCAAGAGTGCGCCC | |
| | AGAAAGGCCAGGCCGTGGATATTGGCAAGGCCGCCTTTACGACCAGCC | |
| | TCAACCTCCTCAGCAAGCTGTTCTTCAGCGTCGAGCTGGCGCACCACA | |
| | AGTCCCATACCAGCCAAGAGTTCAAAGAGCTGATCTGGAACATCATGG | |
| | AAGATATAGGCAAGCCGAACTACGCCGACTACTTCCCGATTCTCGGCT | |
| | GCGTTGACCCATCTGGCATTAGAAGAAGGCTCGCCTGCTCCTTCGACA | |
| | AGCTGATCGCCGTGTTCCAGGGCATCATCTGCGAGAGACTCGCCCCAG | |
| | ATTCCTCCACCACAACTACCACCACCACCGACGACGTGCTCGATGTGC | |
| | TCCTCCAGCTGTTCAAGCAGAACGAGCTGACGATGGGCGAGATCAACC | |
| | ACCTCCTCGTGGACATCTTCGACGCCGGCACCGATACCACATCCTCCA | |
| | CATTCGAGTGGGTGATGACCGAGCTGATCCGCAATCCAGAGATGATGG | |
| | AAAAGGCCCAAGAGGAAATCAAGCAGGTCCTCGGCAAGGACAAGCAGA | |
| | TCCAAGAGTCCGACATCATCAACCTGCCGTACCTCCAGGCGATCATCA | |
| | AAGAGACACTCCGCCTCCATCCGCCGACCGTGTTCTTGCTCCCAAGAA | |
| | AGGCCGACACCGATGTCGAGCTGTACGGCTACATCGTGCCGAAGGATG | |
| | CCCAGATCCTCGTGAACCTCTGGGCCATTGGCAGGGACCCAAACGCCT | |
| | GGCAGAACGCCGATATTTTCAGCCCAGAGCGCTTCATCGGCTGCGAGA | |
| | TCGATGTTAAGGGCCGCGATTTCGGCCTCCTTCCATTTGGCGCTGGCC | |
| | GCAGAATTTGCCCAGGCATGAATCTCGCCATCAGGATGCTCACCCTCA | |
| | TGCTCGCCACACTCCTCCAGTTCTTCAACTGGAAGCTCGAAGGCGACA | |
| | TCTCCCCGAAGGACCTCGACATGGACGAGAAGTTCGGCATTGCGCTCC | |
| | AAAAGACCAAGCCGCTCAAGCTCATCCCGATTCCGCGCTACGGTAGCG | |
| | GAGCTACCAATTTTAGCCTCCTTAAGCAGGCAGGTGATGTAGAAGAGA | |
| | ACCCCGGGCCTATGAAGATGATGAACGGCGAGGACGCCAACGACCAGA | |
| | TGATCAAAGAGTCCTTCTTCATCACCCACGGCAACCCGATCCTCACCG | |
| | TCGAGGATACACATCCGCTCAGGCCGTTCTTCGAGACATGGCGCGAGA | |
| | AGATTTTCTCCAAGAAGCCGAAGGCCATCCTCATCATCTCCGGCCACT | |
| | GGGAGACAGTGAAGCCAACCGTGAACGCCGTGCACATCAACGACACCA | |
| | TCCACGACTTCGACGACTACCCAGCCGCCATGTACCAGTTCAAGTACC | |
| | CAGCTCCAGGCGAGCCAGAGCTTGCGAGAAAGGTGGAAGAGATCCTCA | |
| | AGAAGTCCGGGTTCGAGACAGCCGAGACAGACCAAAAGAGGGGCCTTG | |
| | ATCACGGCGCCTGGGTTCCACTCATGCTCATGTATCCAGAGGCGGACA | |
| | TCCCGGTGTGCCAGCTCTCAGTTCAGCCACATCTCGACGGCACCTACC | |
| | ACTACAATCTCGGCAGAGCCCTCGCGCCGCTCAAGAATGATGGCGTGC | |
| | TCATTATTGGCTCCGGCAGCGCCACACATCCACTCGATGAGACACCGC | |
| | ACTACTTCGATGGTGTTGCCCCTTGGGCCGCTGCCTTCGATTCTTGGC | |
| | TTAGGAAGGCCCTCATCAACGGCCGCTTCGAGGAAGTGAACATCTACG | |
| | AGAGCAAGGCCCCGAACTGGAAGCTCGCCCATCCATTTCCAGAGCACT | |
| | TCTACCCGCTCCACGTTGTGCTCGGCGCTGCTGGTGAAAAGTGGAAGG | |
| | CCGAGCTGATCCACTCCTCCTGGGATCATGGCACACTTTGCCACGGCT | |
| | CCTACAAGTTCACCTCCGCCGGATCCGGAGCAACCAACTTTAGCCTGC | |
| | TCAAGCAAGCAGGAGATGTTGAGGAAAATCCTGGCCCCATGACCGCCA | |
| | TCAAGATGAACACCAACGGCGAGGGCGAGACACAGCACATCCTCATGA | |
| | TCCCGTTCATGGCGCAGGGCCACCTCAGGCCATTTCTCGAACTCGCCA | |
| | TGTTCCTCTACAAGCGCTCCCACGTGATCATCACCCTGCTCACAACTC | |
| | CGCTCAATGCCGGCTTCCTCAGGCACCTCCTTCACCACCATTCCTACT | |
| | CCTCCAGCGGCATCAGGATCGTCGAGCTGCCATTCAACTCCACCAACC | |
| | ACGGACTCCCACCGGGCATCGAGAACACCGATAAGCTCACACTCCCGC | |
| | TCGTGGTGTCCCTCTTCCATTCCACCATCAGCCTCGATCCGCACCTCC | |
| | GCGATTACATCTCCAGGCATTTCAGCCCAGCCAGGCCACCACTCTGCG | |
| | TGATCCATGATGTGTTCCTCGGCTGGGTTGACCAGGTGGCCAAGGATG | |
| | TGGGCTCTACAGGCGTGGTGTTCACAACAGGCGGCGCTTATGGCACAT | |
| | CCGCCTACGTGTCCATCTGGAACGATCTCCCGCACCAGAACTACTCCG | |
| | ACGACCAAGAGTTCCCGCTGCCAGGCTTCCCAGAGAACCATAAGTTCC | |
| | GCAGGTCCCAGCTCCATCGGTTCCTCAGATATGCCGACGGCTCCGACG | |
| | ATTGGTCCAAGTATTTCCAGCCGCAGCTCCGCCAGTCCATGAAGTCTT | |
| | TTGGCTGGCTCTGCAACTCCGTGGAAGAGATCGAGACACTCGGCTTCT | |
| | CCATCCTCCGCAACTACACCAAGCTGCCGATCTGGGGCATCGGCCCAC | |
| | TTATTGCTTCCCCAGTGCAGCACTCCTCCTCCGACAACAATTCAACAG | |
| | GCGCCGAGTTCGTGCAGTGGCTCAGCCTCAAAGAGCCGGACTCCGTCC | |
| | TCTACATCTCCTTCGGCTCCCAGAACACGATCAGCCCGACGCAGATGA | |
| | TGGAACTCGCTGCTGGCCTTGAGTCCTCCGAGAAGCCATTCCTCTGGG | |
| | TGATCAGAGCCCCGTTCGGCTTCGACATCAACGAAGAGATGCGCCCAG | |
| | AGTGGCTGCCAGAGGGCTTTGAGGAACGCATGAAGGTGAAGAAACAGG | |
| | GCAAGCTCGTGTACAAGCTCGGCCCGCAGCTTGAGATCCTCAACCATG | |
| | AATCCATCGGCGGCTTTCTCACCCACTGCGGATGGAACAGCATCCTTG | |
| | AGTCTCTTCGCGAGGGCGTTCCGATGCTTGGATGGCCACTTGCTGCCG | |
| | AGCAGGCCTACAACCTCAAGTACCTCGAAGATGAGATGGGCGTCGCGG | |
| | TTGAGCTTGCTAGAGGCCTCGAAGGCGAGATCTCCAAAGAGAAGGTCA | |
| | AGCGCATCGTCGAGATGATCCTTGAGCGCAACGAGGGCTCCAAAGGCT | |
| | GGGAGATGAAGAATCGCGCCGTGGAAATGGGCAAAAAGCTCAAGGACG | |
| | CCGTGAACGAGGAAAAAGAGCTGAAGGGCTCCTCCGTGAAGGCGATCG | |
| | ACGATTTCCTCGACGCCGTCATGCAGGCCAAACTTGAGCCAAGCCTCC | |
| | AGTGATAGTGAGCTTGGACTCCCATGTTGGCAAAGGCAACCAAACAAA | |
| | CAATGAATGATCCGCTCCTGCATATGGGGCGGTTTGAGTATTTCAACT | |
| | GCCATTTGGGCTGAATTGTAGACATGCTCCTGTCAGAAATTCCGTGAT | |
| | CTTACTCAATATTCAGTAATCTCGGCCAATATCCTAAATGTGCGTGGC | |
| | TTTATCTGTCTTTGTATTGTTTCATCAATTCATGTAACGTTTGCTTTT | |
| | CTTATGAATTTTCAAATAAATTATCGGTAGTCCTGCTTTAATGAGATA | |
| | TGCGAGAAGCCTATGATCGCATGATATTTGCTTTCAATTCTGTTGTGC | |
| | ACGTTGTAAAAAACCTGAGCATGTGTAGCTCAGATCCTTACCGCCGGT | |
| | TTCGGTTCATTCTAATGAATATATCACCCGTTACTATCGTATTTTTAT | |
| | GAATAATATTCTCCGTTCAATTTACTGATTGTACCCTACTACTTATAT | |
| | GTACAATATTAAAATGAAAACAATATATTGTGCTGAATAGGTTTATAG | |
| | CGACATCTATGATAGAGCGCCACAATAACAAACAATTGCGTTTTATTA | |
| | TTACAAATCCAATTTTAAAAAAAGCGGCAGAACCGGTCAAACCTAAAA | |
| | GACTGATTACATAAATCTTATTCAAATTTCAAAAGTGCCCCAGGGGCT | |
| | AGTATCTACGACACACCGAGCGGCGAACTAATAACGCTCACTGAAGGG | |
| | AACTCCGGTTCCCCGCCGGCGCGCATGGGTGAGATTCCTTGAAGTTGA | |
| | GTATTGGCCGTCCGCTCTACCGAAAGTTACGGGCACCATTCAACCCGG | |
| | TCCAGCACGGCGGCCGGGTAACCGACTTGCTGCCCCGAGAATTATGCA | |
| | GCATTTTTTTGGTGTATGTGGGCCCCAAATGAAGTGCAGGTCAAACCT | |
| | TGACAGTGACGACAAATCGTTGGGCGGGTCCAGGGCGAATTTTGCGAC | |
| | AACATGTCGAGGCTCAGCAGGAC | |
| PL-1044 | GGAGGAAGCCAACTAAACAAGACCATAACCATGGTGACATTTGACATA | 161 |
| construct | GTTGTTTACTACTTGCTTGAGCCCCACCCTTGCTTATCGGTTGAACAT | |
| | TACAAGATACACTGCGGGTGGCCTAAGGCACACCGTCCGAAACCGGCA | |
| | AACCAAGCCTGATCGCCGAAATCCAAAATCACTACCGGCAATCTCTAA | |
| | AGTTTATTTCATCCTTATATGACGAGGAAAGAAAAGAAGAGAGAAATA | |
| | ATATCTTAACTTCTAAATCAGTCGCGTCAACTTTCTCGGCTAAGAAAG | |
| | TGAGCACTATCATTTCGCAGACCATGTCATGAGTGCCGACTTGCCATA | |
| | TCTTATTATATTCTTATTTATTTAATTATAATCCCATTGCAATACGTC | |
| | TATTCTATCATGGCCTGCCACTAACGCTCCGTCTAACGTCGTTAAGCC | |
| | ATTGTCATAAGCGGCTGCTCAAAACTCTTCCCGGTGGAGGCGAGGCGT | |
| | TAACGGCGTCTACAAATCTAACGGCCACCAACCATCCAGCCGCCTCTC | |
| | GAAAGCTCCGCTCCGATCGCGGAAATTGCGTGGCGGAGACGAGCGGGC | |
| | TCCTCTCACACGGCCCGGAACCGTCACGGCACGGGTGGGGGATTCCTT | |
| | CCCCAACCCTCCCCACCTCTCCTCCCCCCGTCGCAGCCCATAAATACA | |
| | GGGCCCTCCGCGCCTCTTCCCACAATCTCACATCGTCTCATCGTTCGG | |
| | AGCGCACAACCCCCGGGTTCCAAATCCAAATTGCTCTTCTCGCGACCC | |
| | TCGGCGATCCTTCCCCCGCTTCAAGGTACGGCGATCGTCTCCCCCGTC | |
| | CTCTTGCCCCATCTCCTCGCTCGGCGTGGTTTGGTGGTTCTGCTTGGT | |
| | CTGTGGCTAGGAACTAGGCTGAGGCGTTGACGAAATCATGCTAGATCC | |
| | GCGTGTTTCCTGATCGTGGGTGGCTGGGAGGTGGGGTTTTCGTGTAGA | |
| | TCTGATCGGTTCCGCTGTTTATCCTGTCATGCTCATGTGATTTGTGGG | |
| | GATTTTAGGTCGTTTGTCCGGGAATCGTGGGGTTGCTTCTAGGCTGTT | |
| | CGTAGATGAGATCGTTCTCACGATCTGCTGGGTCGCTGCCTAGGTTCA | |
| | GCTAGGTCTGCCCTGTTTTTGGGTTCGTTTTCGGGATCTGTACGTGCA | |
| | TCTATTATCTGGTTCGATGGTGCTAGCTAGGAACAAACAACTGATTCG | |
| | TCCGATCGATTGTTTTGTTGCCATGTGCAAGGTTAGGTCGTTATCTGA | |
| | TTGCTGTAGATCAGAGTAGAATAAGATCATCACAAGCTAGCTCTTGGG | |
| | CTTATTATGAATCTGCGTTTGTTGCATGATTAAGATGATTATGCTTTT | |
| | TCTTATGCTGCCGTTTGTATATGATGCGGTAGCTTTTAACTGAATAGC | |
| | ACACCTTTCCTGTTTAGTTAGATTAGATTAGATTGCATGATAGATGAG | |
| | GATATATGCTGCTACATCAGTTTGATGATTCTCTGGTACCTCATAATC | |
| | AACTAGCTCATGTGCTTAAATTGAAACTGCATGTGCCACATGATTAAG | |
| | ATGCTAAGATTGGTGAAGATATATACGCTGCTGTTCCTATAGGATCCT | |
| | GTAGCTTTTACCTGGTCAACATGCATCGTCCTGTTATGGATAGATATG | |
| | CATGATAGATGAAGATATGTACTGCTACAATTTGATGATTCTTTTGTG | |
| | CACCTGATGATCATGCATGCTCTTTGCCCTTACTTTGATATACTTGGA | |
| | TGATGGCATGCTTAGTACTAATGATGTGATGAACACACATGACCTGTT | |
| | GGTATGAATATGATGTTGCTGTTTGCTTGTGATGAGTTCTGTTTGTTT | |
| | ACTGCTAGGCACTTACCCTGTTGTCTGGTTCTCTTTTGCAGAATGGTA | |
| | GAACAAGACGGGCTGCATGCTGGATCACCTGCGGCCTGGGTCGAAAGG | |
| | CTATTCGGTTATGACTGGGCGCAGCAAACCATTGGGTGCAGCGATGCA | |
| | GCCGTCTTTCGACTCAGCGCCCAAGGTAGACCCGTATTGTTTGTGAAA | |
| | ACTGACTTATCCGGGGCACTGAATGAATTACAAGACGAAGCAGCCAGA | |
| | CTTAGTTGGTTAGCGACCACAGGTGTGCCCTGTGCAGCGGTCCTTGAC | |
| | GTAGTTACCGAGGCAGGACGGGACTGGTTATTGCTTGGAGAAGTACCG | |
| | GGCCAAGACCTGTTATCTAGCCACCTCGCCCCGGCTGAAAAAGTCTCA | |
| | ATTATGGCGGATGCTATGAGGCGACTTCATACATTGGACCCAGCCACG | |
| | TGTCCCTTTGATCATCAGGCGAAACACAGAATAGAACGTGCAAGGACA | |
| | CGAATGGAGGCTGGGCTCGTTGATCAAGACGATTTAGATGAAGAGCAT | |
| | CAAGGCTTGGCACCTGCTGAGTTATTTGCTCGGTTAAAAGCTAGAATG | |
| | CCAGACGGGGAAGATCTTGTCGTAACCCATGGCGATGCCTGCTTGCCA | |
| | AATATTATGGTGGAGAATGGTCGTTTCTCAGGGTTTATCGATTGTGGG | |
| | CGACTTGGCGTGGCCGACAGGTATCAAGATATAGCCCTAGCAACTAGA | |
| | GACATAGCCGAAGAGTTGGGTGGGGAGTGGGCTGACAGATTCCTCGTG | |
| | CTATACGGGATTGCCGCTCCCGATTCCCAGAGAATCGCATTTTATCGA | |
| | CTCTTAGACGAATTCTTTTAGGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTGCAAGAA | |
| | TTCAAGCTTGGAGGAAGAACTCGAGAGGGAATTGCAGATCATGAGGCA | |
| | GATGGCTATTTTTGTGTCACATATGCGCAAAAAGAGAGGCTATATTTG | |
| | TGTCCCTAGGTTCTTCGTTGTATTGCAGTTTCCATATCAATCTGACTT | |
| | GGTCGCATGAGAAATTGATGGTTAAATAATTTGAATCTCTCATGTAGT | |
| | ATCAACTATTAGATATTATTTTCACCAAATATATTTCCATCGGAGAAG | |
| | AAGAGGCTACAGAGGAAGCAGAAGAGAGGGGTGGGAGAATTTTTACAC | |
| | TTTTGTACACCCACTTAAACAGCAAAATCCGTATGAAAACAGGCCCAC | |
| | CAAAACAATGCCACGATAACAATCCGTAGAAACAAAAGCTTCATTTAA | |
| | CAGCGGCGCAACAAAGCACGCTTATCCATGGTAGTTGTAGTCCGTATG | |
| | CGATCCAAAGATCACGATTCACGCGTGACGGACGGACGACGCGTGCCA | |
| | CACCACAACTAACGGCATCCATGGTAGTTGTAGTCCGTATGCGATCCA | |
| | AAGATCACGATTCACGCGTGACGGACGGACGACGCGCGCCACACCACA | |
| | ACTAACAGCGTGAGCCAGCGTCCAAACTCCGGATGGCAACGGGGACGA | |
| | AACCCGTCGGGTAGTCACTGCCCAAACCCGTCCCCGCAACCTTCATCC | |
| | CAAACCCGTCCCCGTTTCCGGTCGCGGGTTTCAGTTTTCTACCAGACC | |
| | CGTCCCCATCGGGTTTTTCATCCCCGTCGGGAAATCCGAACCCGCCAG | |
| | CATTTCAGCACCAAGCCAAAGTTGCAGCAGCAACATGAATAAAAAACA | |
| | ACCCGTTTCAACACCAAGATAAAACAAAACATTATAATTTAGACAACA | |
| | TTTCACACGTATAACAATAACATATAGTTCTCACATATAACAACACCA | |
| | TTTCACACATAAAACAACACCATTTGGGATAAAAATATGGGCTATATC | |
| | AGGCCATTTTTATGGGCCATATTGAGTTTTCGTGGGTTTCACAGGTAC | |
| | CGGATTTGTAGAATGCTGAACCGGGTTTGAACCGTAAAATCCGCGGGT | |
| | ATTGAATTTGACCCAATCCCGTCGTCCCCTGGTGGGGTAAAAACACCA | |
| | TCTTGAGTCCAAACGGCCACCAACCAAACTCCGACGGCAACAAACAAA | |
| | CGGCGTTGCTTTGCTCCTCGGTATCTCCGTGACCGCTCAATCTCCCGG | |
| | CTGTTTCCCCGGAATTGCGTGGACTCTCTCATCCACACGCAAACCGCC | |
| | TCTCCCTCCTCTCTCGTCCTATCCGCCCCGGTGCCGTAGCCTCACGGG | |
| | ACTCTTCTTCCTCCCTTGCTATAAAATCCCCGCCCCCTCCCGTCTCCT | |
| | CTCCACACATCCAAACTCTCAATCGCACCGAGAAAAATCTCCTAGCGA | |
| | TCGAAGCGAAGCCTCTCCCGATCCTCTCAAGGTACGCCCGTTTCCCGT | |
| | CGATCCTCCTCCTTCCGTTCGTGTTCTGTAGCCGATCGATTCGATTCC | |
| | CTTACACCCGTTCGTGTTCTCTCGTGGATCGATCGATTGTTTGTTGCT | |
| | AGAAGGAACTCGTAGATCTGGCGTTTATGAACTGTGATTCGGGTTAGT | |
| | CCAGATCGATTCAGGTCGGTCGTCGTTGAGCCTCTCGGCTATGTCTGG | |
| | ATTATCGTGTAGATCTGCTGGTTCAGTTGATTATGTTCTTCTAGGAGT | |
| | AATTTCGTTGGGTCAGCGCGATTTCTGCTTAATCTATGCTGCTTATTG | |
| | CGCCTGTACCTATCTACTAAGCTATGTGCACCTGTAATTTTGCTAGAT | |
| | TATTCGTTCATCCTCGTAGTTGGTTTGTCACAGTAATCCGTATGGGTT | |
| | CTGACGATGTTATTGTTGGTCATACCTAGGCTTCTCCAGATTTTATTT | |
| | TGTTAAAATTGGATAGATCTGCTACTGATAGTTGATGATGGAATTTGG | |
| | TGCTGAATCTATGCTATTTATTGCGCCTATACCTGATCTATCGGGCTA | |
| | TGTACGGCTGTAGTTTACTGGATTATTCGTTCATCCTCGGTAGTTGGT | |
| | TCATCGTTTGGGTTCTGACGATAATATTGTTGATTATGCGTAGGCTTC | |
| | TGCAGATTGTTGTTAAAATTGGATACATCGGTTACTGATGGTTGATGA | |
| | TAGATTTGTGCTGAACCTATCTGTTTATTGCTCCTATACCTGATCTAT | |
| | AGGGCTATGTATGCCTGTAATTTACCAGATTATTCGTTCATCCTCGTA | |
| | GTTGGTTCATCTCTATAATTCGTATGGGTTCTTATGATGTTATCGTTG | |
| | ATTATGCCTAGTCTTATACAGATTATTGTGTCAAGATTGAATATACCT | |
| | GCTACTGATCGGTGATAATTTGGTTAGTAGTTTGCAATCTGCTAGGAA | |
| | CACGTTACCACTGTAATCTGTAAACATGGTTTGCCAGAGTAGTTTGTT | |
| | CTACTACTCTTGATATGGTTGCTGATTTTAGTCGCCTCCTTTTGGATC | |
| | ATGTATTGATGTCCTTGCAGATTTCCGTGTACTTACCCCGGCTTTTGT | |
| | GTACTTCGTGTTAACAGAATGGATCATGCGACCCTCGCCATGATCCTC | |
| | GCGATCTGGTTCATCAGCTTCCACTTCATCAAGCTGCTGTTCTCCCAG | |
| | CAGACCACCAAGCTGCTTCCGCCAGGACCAAAGCCGCTTCCGATCATC | |
| | GGCAACATCCTTGAGGTGGGCAAGAAGCCGCATCGGTCCTTCGCCAAC | |
| | CTCGCCAAGATTCACGGCCCACTCATTTCCCTCAGACTCGGCTCTGTG | |
| | ACCACCATCGTTGTGTCCTCTGCCGACGTGGCCAAAGAGATGTTCCTC | |
| | AAGAAGGATCACCCGCTCTCCAACCGCACGATCCCGAATAGTGTTACA | |
| | GCCGGCGACCACCACAAGCTCACCATGTCTTGGCTCCCGGTGTCTCCG | |
| | AAGTGGCGCAACTTCCGCAAGATTACCGCCGTGCATCTGCTCTCCCCA | |
| | CAGAGACTCGATGCCTGCCAGACATTCAGGCACGCCAAGGTGCAGCAG | |
| | CTCTACGAGTACGTTCAAGAGTGCGCCCAGAAAGGCCAGGCCGTGGAT | |
| | ATTGGCAAGGCCGCCTTTACGACCAGCCTCAACCTCCTCAGCAAGCTG | |
| | TTCTTCAGCGTCGAGCTGGCGCACCACAAGTCCCATACCAGCCAAGAG | |
| | TTCAAAGAGCTGATCTGGAACATCATGGAAGATATAGGCAAGCCGAAC | |
| | TACGCCGACTACTTCCCGATTCTCGGCTGCGTTGACCCATCTGGCATT | |
| | AGAAGAAGGCTCGCCTGCTCCTTCGACAAGCTGATCGCCGTGTTCCAG | |
| | GGCATCATCTGCGAGAGACTCGCCCCAGATTCCTCCACCACAACTACC | |
| | ACCACCACCGACGACGTGCTCGATGTGCTCCTCCAGCTGTTCAAGCAG | |
| | AACGAGCTGACGATGGGCGAGATCAACCACCTCCTCGTGGACATCTTC | |
| | GACGCCGGCACCGATACCACATCCTCCACATTCGAGTGGGTGATGACC | |
| | GAGCTGATCCGCAATCCAGAGATGATGGAAAAGGCCCAAGAGGAAATC | |
| | AAGCAGGTCCTCGGCAAGGACAAGCAGATCCAAGAGTCCGACATCATC | |
| | AACCTGCCGTACCTCCAGGCGATCATCAAAGAGACACTCCGCCTCCAT | |
| | CCGCCGACCGTGTTCTTGCTCCCAAGAAAGGCCGACACCGATGTCGAG | |
| | CTGTACGGCTACATCGTGCCGAAGGATGCCCAGATCCTCGTGAACCTC | |
| | TGGGCCATTGGCAGGGACCCAAACGCCTGGCAGAACGCCGATATTTTC | |
| | AGCCCAGAGCGCTTCATCGGCTGCGAGATCGATGTTAAGGGCCGCGAT | |
| | TTCGGCCTCCTTCCATTTGGCGCTGGCCGCAGAATTTGCCCAGGCATG | |
| | AATCTCGCCATCAGGATGCTCACCCTCATGCTCGCCACACTCCTCCAG | |
| | TTCTTCAACTGGAAGCTCGAAGGCGACATCTCCCCGAAGGACCTCGAC | |
| | ATGGACGAGAAGTTCGGCATTGCGCTCCAAAAGACCAAGCCGCTCAAG | |
| | CTCATCCCGATTCCGCGCTACGGTAGCGGAGCTACCAATTTTAGCCTC | |
| | CTTAAGCAGGCAGGTGATGTAGAAGAGAACCCCGGGCCTATGAAGATG | |
| | ATGAACGGCGAGGACGCCAACGACCAGATGATCAAAGAGTCCTTCTTC | |
| | ATCACCCACGGCAACCCGATCCTCACCGTCGAGGATACACATCCGCTC | |
| | AGGCCGTTCTTCGAGACATGGCGCGAGAAGATTTTCTCCAAGAAGCCG | |
| | AAGGCCATCCTCATCATCTCCGGCCACTGGGAGACAGTGAAGCCAACC | |
| | GTGAACGCCGTGCACATCAACGACACCATCCACGACTTCGACGACTAC | |
| | CCAGCCGCCATGTACCAGTTCAAGTACCCAGCTCCAGGCGAGCCAGAG | |
| | CTTGCGAGAAAGGTGGAAGAGATCCTCAAGAAGTCCGGGTTCGAGACA | |
| | GCCGAGACAGACCAAAAGAGGGGCCTTGATCACGGCGCCTGGGTTCCA | |
| | CTCATGCTCATGTATCCAGAGGCGGACATCCCGGTGTGCCAGCTCTCA | |
| | GTTCAGCCACATCTCGACGGCACCTACCACTACAATCTCGGCAGAGCC | |
| | CTCGCGCCGCTCAAGAATGATGGCGTGCTCATTATTGGCTCCGGCAGC | |
| | GCCACACATCCACTCGATGAGACACCGCACTACTTCGATGGTGTTGCC | |
| | CCTTGGGCCGCTGCCTTCGATTCTTGGCTTAGGAAGGCCCTCATCAAC | |
| | GGCCGCTTCGAGGAAGTGAACATCTACGAGAGCAAGGCCCCGAACTGG | |
| | AAGCTCGCCCATCCATTTCCAGAGCACTTCTACCCGCTCCACGTTGTG | |
| | CTCGGCGCTGCTGGTGAAAAGTGGAAGGCCGAGCTGATCCACTCCTCC | |
| | TGGGATCATGGCACACTTTGCCACGGCTCCTACAAGTTCACCTCCGCC | |
| | GGATCCGGAGCAACCAACTTTAGCCTGCTCAAGCAAGCAGGAGATGTT | |
| | GAGGAAAATCCTGGCCCCATGACCGCCATCAAGATGAACACCAACGGC | |
| | GAGGGCGAGACACAGCACATCCTCATGATCCCGTTCATGGCGCAGGGC | |
| | CACCTCAGGCCATTTCTCGAACTCGCCATGTTCCTCTACAAGCGCTCC | |
| | CACGTGATCATCACCCTGCTCACAACTCCGCTCAATGCCGGCTTCCTC | |
| | AGGCACCTCCTTCACCACCATTCCTACTCCTCCAGCGGCATCAGGATC | |
| | GTCGAGCTGCCATTCAACTCCACCAACCACGGACTCCCACCGGGCATC | |
| | GAGAACACCGATAAGCTCACACTCCCGCTCGTGGTGTCCCTCTTCCAT | |
| | TCCACCATCAGCCTCGATCCGCACCTCCGCGATTACATCTCCAGGCAT | |
| | TTCAGCCCAGCCAGGCCACCACTCTGCGTGATCCATGATGTGTTCCTC | |
| | GGCTGGGTTGACCAGGTGGCCAAGGATGTGGGCTCTACAGGCGTGGTG | |
| | TTCACAACAGGCGGCGCTTATGGCACATCCGCCTACGTGTCCATCTGG | |
| | AACGATCTCCCGCACCAGAACTACTCCGACGACCAAGAGTTCCCGCTG | |
| | CCAGGCTTCCCAGAGAACCATAAGTTCCGCAGGTCCCAGCTCCATCGG | |
| | TTCCTCAGATATGCCGACGGCTCCGACGATTGGTCCAAGTATTTCCAG | |
| | CCGCAGCTCCGCCAGTCCATGAAGTCTTTTGGCTGGCTCTGCAACTCC | |
| | GTGGAAGAGATCGAGACACTCGGCTTCTCCATCCTCCGCAACTACACC | |
| | AAGCTGCCGATCTGGGGCATCGGCCCACTTATTGCTTCCCCAGTGCAG | |
| | CACTCCTCCTCCGACAACAATTCAACAGGCGCCGAGTTCGTGCAGTGG | |
| | CTCAGCCTCAAAGAGCCGGACTCCGTCCTCTACATCTCCTTCGGCTCC | |
| | CAGAACACGATCAGCCCGACGCAGATGATGGAACTCGCTGCTGGCCTT | |
| | GAGTCCTCCGAGAAGCCATTCCTCTGGGTGATCAGAGCCCCGTTCGGC | |
| | TTCGACATCAACGAAGAGATGCGCCCAGAGTGGCTGCCAGAGGGCTTT | |
| | GAGGAACGCATGAAGGTGAAGAAACAGGGCAAGCTCGTGTACAAGCTC | |
| | GGCCCGCAGCTTGAGATCCTCAACCATGAATCCATCGGCGGCTTTCTC | |
| | ACCCACTGCGGATGGAACAGCATCCTTGAGTCTCTTCGCGAGGGCGTT | |
| | CCGATGCTTGGATGGCCACTTGCTGCCGAGCAGGCCTACAACCTCAAG | |
| | TACCTCGAAGATGAGATGGGCGTCGCGGTTGAGCTTGCTAGAGGCCTC | |
| | GAAGGCGAGATCTCCAAAGAGAAGGTCAAGCGCATCGTCGAGATGATC | |
| | CTTGAGCGCAACGAGGGCTCCAAAGGCTGGGAGATGAAGAATCGCGCC | |
| | GTGGAAATGGGCAAAAAGCTCAAGGACGCCGTGAACGAGGAAAAAGAG | |
| | CTGAAGGGCTCCTCCGTGAAGGCGATCGACGATTTCCTCGACGCCGTC | |
| | ATGCAGGCCAAACTTGAGCCAAGCCTCCAGTGATAGTGAGCTTCTCTA | |
| | GCTAGAGTCGATCGACAAGCTCGAGTTTCTCCATAATAATGTGTGAGT | |
| | AGTTCCCAGATAAGGGAATTAGGGTTCCTATAGGGTTTCGCTCATGTG | |
| | TTGAGCATATAAGAAACCCTTAGTATGTATTTGTATTTGTAAAATACT | |
| | TCTATCAATAAAATTTCTAATTCCTAAAACCAAAATCCAGTACTAAAA | |
| | TCCAGATGGTAGTCCTGCTTTAATGAGATATGCGAGAAGCCTATGATC | |
| | GCATGATATTTGCTTTCAATTCTGTTGTGCACGTTGTAAAAAACCTGA | |
| | GCATGTGTAGCTCAGATCCTTACCGCCGGTTTCGGTTCATTCTAATGA | |
| | ATATATCACCCGTTACTATCGTATTTTTATGAATAATATTCTCCGTTC | |
| | AATTTACTGATTGTACCCTACTACTTATATGTACAATATTAAAATGAA | |
| | AACAATATATTGTGCTGAATAGGTTTATAGCGACATCTATGATAGAGC | |
| | GCCACAATAACAAACAATTGCGTTTTATTATTACAAATCCAATTTTAA | |
| | AAAAAGCGGCAGAACCGGTCAAACCTAAAAGACTGATTACATAAATCT | |
| | TATTCAAATTTCAAAAGTGCCCCAGGGGCTAGTATCTACGACACACCG | |
| | AGCGGCGAACTAATAACGCTCACTGAAGGGAACTCCGGTTCCCCGCCG | |
| | GCGCGCATGGGTGAGATTCCTTGAAGTTGAGTATTGGCCGTCCGCTCT | |
| | ACCGAAAGTTACGGGCACCATTCAACCCGGTCCAGCACGGCGGCCGGG | |
| | TAACCGACTTGCTGCCCCGAGAATTATGCAGCATTTTTTTGGTGTATG | |
| | TGGGCCCCAAATGAAGTGCAGGTCAAACCTTGACAGTGACGACAAATC | |
| | GTTGGGCGGGTCCAGGGCGAATTTTGCGACAACATGTCGAGGCTCAGC | |
| | AGGACCGCTACTAGAATTCGAGCTCGGAGAGGAAGAAGTTCCCAATCT | |
| | TGAAATATCGAAGGTTCGCCTTTCTGTTGTAATATTGCTCAATCCTCT | |
| | ACTTTTGTGCCGCCATCTGTATCAAAGTTGCTTCCCTTATCTCCTTTG | |
| | TCAAATCTAAATTTGTTAAAAGTTCTTTCTCATTTGTTACTTCATTTG | |
| | TATGTACGTATCTTGTTCTTGGCTCTCCTATTTCCACCGAAATTAAGG | |
| | CCTCAGTACCATAAATAAGTGAGAATGGTGTTTCTCTTGTGCTTATCT | |
| | TCGTCGTTGTCCTGTAATCCCATACTAGCCCAAGTAGTACTTCAGGGA | |
| | ACCTGCCTTTTGATGATTCTAACCGTTTCTTTATATTGTTGACAATAA | |
| | TTGTATTTGTCGATTATGGTTTCCCATTAGCTATGGGATGGCAGTGCG | |
| | CCGAAATAATTTGTTTAATTTCCCAGATTTTGAGAAAATCGGTAACTT | |
| | TTGAACCTACGAATTGTGGCCCATTATCGCGTACTATTTTTCTTGGGA | |
| | CACAAAATCGACATATTATGTTCCTCCAAATAATATCAACAACCTCTT | |
| | TCTCTCGATCTATTTAAAGGCACATGCTTCTACCCACATAGAAAAATA | |
| | ATCAGTTAAGATTAATAAAAGTCGTTCCTTTCCTGGTGCTTTAGGTAG | |
| | AGGACCTACAATACATAACCCACTTCATAAACGGCCACGGTGCTATGA | |
| | TTGAATGAAGAAACACTGCTGGTTGATGCATATTGTTTGCACCCCGTT | |
| | GACATTTATCACACCTTCTAATAAAAATTTTCGCTTTGTGTTCTATTT | |
| | TTTGCAAATAATACCGCCCCTTTTAGCGTCTTTAAAATGACCTTCCTC | |
| | CAACGTGATTGCAACAATGTTCTTTATGTACTTCTTGCATCACATATT | |
| | CAGTTTGTGATAGACCAAGGCACCGTGCTAAGGGTCCCATGAACATTT | |
| | CGATACAAATTTTCTTCTATTAAGCAATATTGAGCAGCTTTCACTCGC | |
| | AATAATTGGGATTTTTAATCGACGCTTTACGAATTCCCATGGGGAGCT | |
| | GCAGTGCAGCGTGACCCGGTCGTGCCCCTCTCTAGAGATAATGAGCAT | |
| | TGCATGTCTAAGTTATAAAAAATTACCACATATTTTTTTTGTCACACT | |
| | TGTTTGAAGTGCAGTTTATCTATCTTTATACATATATTTAAACTTTAC | |
| | TCTACGAATAATATAATCTATAGTACTACAATAATATCAGTGTTTTAG | |
| | AGAATCATATAAATGAACAGTTAGACATGGTCTAAAGGACAATTGAGT | |
| | ATTTTGACAACAGGACTCTACAGTTTTATCTTTTTAGTGTGCATGTGT | |
| | TCTCCTTTTTTTTTGCAAATAGCTTCACCTATATAATACTTCATCCAT | |
| | TTTATTAGTACATCCATTTAGGGTTTAGGGTTAATGGTTTTTATAGAC | |
| | TAATTTTTTTAGTACATCTATTTTATTCTATTTTAGCCTCTAAATTAA | |
| | GAAAACTAAAACTCTATTTTAGTTTTTTTATTTAATAATTTAGATATA | |
| | AAATAGAATAAAATAAAGTGACTAAAAATTAAACAAATACCCTTTAAG | |
| | AAATTAAAAAAACTAAGGAAACATTTTTCTTGTTTCGAGTAGATAATG | |
| | CCAGCCTGTTAAACGCCGTCGACGAGTCTAACGGACACCAACCAGCGA | |
| | ACCAGCAGCGTCGCGTCGGGCCAAGCGAAGCAGACGGCACGGCATCTC | |
| | TGTCGCTGCCTCTGGACCCCTCTCGAGAGTTCCGCTCCACCGTTGGAC | |
| | TTGCTCCGCTGTCGGCATCCAGAAATTGCGTGGCGGAGCGGCAGACGT | |
| | GAGCCGGCACGGCAGGCGGCCTCCTCCTCCTCTCACGGCACCGGCAGC | |
| | TACGGGGGATTCCTTTCCCACCGCTCCTTCGCTTTCCCTTCCTCGCCC | |
| | GCCGTAATAAATAGACACCCCCTCCACACCCTCTTTCCCCAACCTCGT | |
| | GTTGTTCGGAGCGCACACACACACAACCAGATCTCCCCCAAATCCACC | |
| | CGTCGGCACCTCCGCTTCAAGGTACGCCGCTCGTCCTCCCCCCCCCCC | |
| | CTCTCTACCTTCTCTAGATCGGCGTTCCGGTCCATGGTTAGGGCCCGG | |
| | TAGTTCTACTTCTGTTCATGTTTGTGTTAGATCCGTGTTTGTGTTAGA | |
| | TCCGTGCTGCTAGCGTTCGTACACGGATGCGACCTGTACGTCAGACAC | |
| | GTTCTGATTGCTAACTTGCCAGTGTTTCTCTTTGGGGAATCCTGGGAT | |
| | GGCTCTAGCCGTTCCGCAGACGGGATCGATTTCATGATTTTTTTTGTT | |
| | TCGTTGCATAGGGTTTGGTTTGCCCTTTTCCTTTATTTCAATATATGC | |
| | CGTGCACTTGTTTGTCGGGTCATCTTTTCATGCTTTTTTTTGTCTTGG | |
| | TTGTGATGATGTGGTCTGGTTGGGCGGTCGTTCTAGATCGGAGTAGAA | |
| | TTCTGTTTCAAACTACCTGGTGGATTTATTAATTTTGGATCTGTATGT | |
| | GTGTGCCATACATATTCATAGTTACGAATTGAAGATGATGGATGGAAA | |
| | TATCGATCTAGGATAGGTATACATGTTGATGCGGGTTTTACTGATGCA | |
| | TATACAGAGATGCTTTTTGTTCGCTTGGTTGTGATGATGTGGTGTGGT | |
| | TGGGCGGTCGTTCATTCGTTCTAGATCGGAGTAGAATACTGTTTCAAA | |
| | CTACCTGGTGTATTTATTAATTTTGGAACTGTATGTGTGTGTCATACA | |
| | TCTTCATAGTTACGAGTTTAAGATGGATGGAAATATCGATCTAGGATA | |
| | GGTATACATGTTGATGTGGGTTTTACTGATGCATATACATGATGGCAT | |
| | ATGCAGCATCTATTCATATGCTCTAACCTTGAGTACCTATCTATTATA | |
| | ATAAACAAGTATGTTTTATAATTATTTTGATCTTGATATACTTGGATG | |
| | ATGGCATATGCAGCAGCTATATGTGGATTTTTTTAGCCCTGCCTTCAT | |
| | ACGCTATTTATTTGCTTGGTACTGTTTCTTTTGTCGATGCTCACCCTG | |
| | TTGTTTGGTGTTACTTCTGCAGAATGATATCACTATCCTCTTTTCATC | |
| | CATCAAGCACAACTGCTACCGCTACCGCAGCTGCCGCAACCACACACC | |
| | CTCCACAGCAATGCCCCGCATTCTCTTCACCACCTTCGCATCTATCCT | |
| | TGCCGCTACGACACCCTCGTCAACATCTTGTGGTTAGGTGTGGAGGGG | |
| | GTGGCTCTGCATCGGAGTCCGTATTCAACAGAGATTCAGCCGCAACTA | |
| | GGGTTTCAAACGATCACTTGGATGTATCTAAAAGGGATGTAAAACTCA | |
| | AAATTGCTATCATAGGGTTCGGAAACTTTGGGCAATTTCTCGCAAAGA | |
| | CTATGGCAAAACAAGGTCATAGAGTACTAGCATATTCTCGATCAGACT | |
| | ATTCCCGGGCGGCAAAAGAGATTGGAGTTGAATATTTTACTGATGCAG | |
| | ATGACTTGTGTGAGGAACATCCTGAAGTAATTCTTTTGTGTACATCTA | |
| | TACTGTCTACTGAGAAGGTGTTGCGGTCATTACCTCTCCATAGATTGC | |
| | GCAGATCAACCCTGTTCGCTGATGTTTTGAGTGTGAAAGAGTTCCCTC | |
| | GCTCTTTATTTCTGCAACTCTTGCCCAAAGATTTTGATATACTCTGTA | |
| | CCCACCCTATGTTTGGGCCAGACAGCGGGAAAGACGGGTGGGGCGGAC | |
| | TCCCATTCGTATTTGACAAAGTAAGGGTGGGCAGCGACCAATCTCGGA | |
| | CGTCAAGAGCTGAGGCATTTTTAGATGTTTTTAGAAACGCCGGGTGTA | |
| | GAATGGTGGAAATGTCATGCGTAGATCATGACAAACATGCCGCAGGCT | |
| | CACAATTTATTACACACATGATGGGAAGGGTACTGGAAAAACTAGCAC | |
| | TTGAAAATACGCCAATTAACACAAAGGGATACGAATCATTACTAAATC | |
| | TAGTTGATAACACAGCACGCGACAGCTTTGAATTGTTTTACGGCCTAT | |
| | TCCTTTATAACAAGAATGCTATGGAGCAGTTGGATAGAATGGACTGGG | |
| | CTTTTGAGATGGTGAAAAAGCAGCTAAGCGGTTATTTGCATGATCTGG | |
| | TGAGGAAACAACTAATGCTGGAAGGGAACAATGATCAGGCGGAGGTCA | |
| | CCTTCGATAAGCCCCTGATGCTACCCTCTCCCACAATTAACCCACCTC | |
| | AAATAGTGCCCTCCGCTGACATGGCTGAGAAAAAGCACGATCTTGTAG | |
| | TTGTCAATGGGACTAGGATGGCTTCTATGATATCCTCTTCAGCTGTGA | |
| | CTACAGTCAGCCGTGCTTCTACGGTGCAATCGGCCGCGGTGGCTCCAT | |
| | TCGGCGGCCTCAAATCCATGACTGGATTCCCAGTTAAGAAGGTCAACA | |
| | CTGACATTACTTCCATTACAAGCAATGGTGGAAGAGTAAAGTGCATGC | |
| | AGATGAATTACCAGAACGATGACTTAAGAATCAAAGAAATTAAAGAGT | |
| | TGTTACCACCTGTGGCTCTTTTGGAAAAATTCCCAGCAACTGAGAATG | |
| | CTGCAAACACAGTTGCTCATGCAAGAAAGGCTATTCACAAAATCTTGA | |
| | AGGGTAATGATGACAGGTTACTTGTTGTGATCGGACCATGCTCAATAC | |
| | ATGATCCTGTTGCTGCAAAGGAATACGCTACTAGATTGCTTGCATTGA | |
| | GGGAAGAGTTAAAGGATGAACTTGAGATTGTTATGAGAGTGTACTTCG | |
| | AGAAACCAAGGACCACTGTTGGTTGGAAGGGACTTATCAATGATCCTC | |
| | ACATGGACAACTCCTTCCAAATTAATGATGGTTTGAGAATCGCTAGGA | |
| | AACTTTTGCTTGATATTAACGACTCAGGTTTGCCAGCTGCAGGAGAAT | |
| | TTTTAGATATGATCACACCTCAGTACTTAGCTGACCTTATGTCATGGG | |
| | GTGCTATAGGAGCAAGAACAACCGAAAGTCAAGTTCATAGGGAGCAGG | |
| | CTTCCGGTTTGTCTTGTCCAGTGGGATTCAAAAATGGTACTGATGGAA | |
| | CAATTAAGGTTGCTATAGACGCAATTAACGCTGCAGGTGCTCCTCATT | |
| | GTTTTCTTTCTGTTACAAAATGGGGACACTCAGCAATCGTGAATACCA | |
| | GTGGTAACGGAGATTGCCATATTATCTTGAGAGGTGGAAAAGAACCAA | |
| | ATTATTCAGCTAAGCACGTTGCAGAAGTGAAAGAGGGTTTGAACAAGG | |
| | CTGGATTACCTGCACAAGTTATGATCGATTTCTCTCATGCTAACTCCT | |
| | CTAAGCAATTCAAGAAACAGATGGATGTTTGTGCTGACGTGTGCCAAC | |
| | AGATCGCTGGTGGAGAAAAGGCTATTATTGGTGTTATGGTGGAAAGTC | |
| | ACTTAGTTGAGGGAAATCAATCATTAGAAAGTGGAGAGCCTCTTGCTT | |
| | ACGGAAAATCTATTACCGATGCATGCATCGGTTGGGAAGATACTGACG | |
| | CTCTTTTGAGACAGTTGGCTAACGCAGTTAAGGCAAGAAGGGGTTGAT | |
| | GAGCTTGGACTCCCATGTTGGCAAAGGCAACCAAACAAACAATGAATG | |
| | ATCCGCTCCTGCATATGGGGCGGTTTGAGTATTTCAACTGCCATTTGG | |
| | GCTGAATTGTAGACATGCTCCTGTCAGAAATTCCGTGATCTTACTCAA | |
| | TATTCAGTAATCTCGGCCAATATCCTAAATGTGCGTGGCTTTATCTGT | |
| | CTTTGTATTGTTTCATCAATTCATGTAACGTTTGCTTTTCTTATGAAT | |
| | TTTCAAATAAATTATCGGTAGTCCTGCTTTAATGAGATATGCGAGAAG | |
| | CCTATGATCGCATGATATTTGCTTTCAATTCTGTTGTGCACGTTGTAA | |
| | AAAACCTGAGCATGTGTAGCTCAGATCCTTACCGCCGGTTTCGGTTCA | |
| | TTCTAATGAATATATCACCCGTTACTATCGTATTTTTATGAATAATAT | |
| | TCTCCGTTCAATTTACTGATTGTACCCTACTACTTATATGTACAATAT | |
| | TAAAATGAAAACAATATATTGTGCTGAATAGGTTTATAGCGACATCTA | |
| | TGATAGAGCGCCACAATAACAAACAATTGCGTTTTATTATTACAAATC | |
| | CAATTTTAAAAAAAGCGGCAGAACCGGTCAAACCTAAAAGACTGATTA | |
| | CATAAATCTTATTCAAATTTCAAAAGTGCCCCAGGGGCTAGTATCTAC | |
| | GACACACCGAGCGGCGAACTAATAACGCTCACTGAAGGGAACTCCGGT | |
| | TCCCCGCCGGCGCGCATGGGTGAGATTCCTTGAAGTTGAGTATTGGCC | |
| | GTCCGCTCTACCGAAAGTTACGGGCACCATTCAACCCGGTCCAGCACG | |
| | GCGGCCGGGTAACCGACTTGCTGCCCCGAGAATTATGCAGCATTTTTT | |
| | TGGTGTATGTGGGCCCCAAATGAAGTGCAGGTCAAACCTTGACAGTGA | |
| | CGACAAATCGTTGGGCGGGTCCAGGGCGAATTTTGCGACAACATGTCG | |
| | AGGCTCAGCAGGAC | |
| PL-1233 | GTGATCCGTTTAAACTATCAGTGTTTGACAGGATATATTGGCGGGTAA | 162 |
| construct | ACCTAAGAGAAAAGAGCGTTTATTAGAATAATCGGATATTTAAAAGGG | |
| | CGTGAAAAGGTTTATCCGTTCGTCCATTTGTATGTGCCAGCCGCCTTT | |
| | GCGACGCTCACCGGGCTGGTTGCCCTCGCCGCTGGGCTGGCGGCCGTC | |
| | TATGGCCCTGCAAACGCGCCAGAAACGCCGTCGAAGCCGTGTGCGAGA | |
| | CACCGCGGCCGCCGGCGTTGTGGATACCTCGCGGAAAACTTGGCCCTC | |
| | ACTGACAGATGAGGGGCGGACGTTGACACTTGAGGGGCCGACTCACCC | |
| | GGCGCGGCGTTGACAGATGAGGGGCAGGCTCGATTTCGGCCGGCGACG | |
| | TGGAGCTGGCCAGCCTCGCAAATCGGCGAAAACGCCTGATTTTACGCG | |
| | AGTTTCCCACAGATGATGTGGACAAGCCTGGGGATAAGTGCCCTGCGG | |
| | TATTGACACTTGAGGGGCGCGACTACTGACAGATGAGGGGCGCGATCC | |
| | TTGACACTTGAGGGGCAGAGTGCTGACAGATGAGGGGCGCACCTATTG | |
| | ACATTTGAGGGGCTGTCCACAGGCAGAAAATCCAGCATTTGCAAGGGT | |
| | TTCCGCCCGTTTTTCGGCCACCGCTAACCTGTCTTTTAACCTGCTTTT | |
| | AAACCAATATTTATAAACCTTGTTTTTAACCAGGGCTGCGCCCTGTGC | |
| | GCGTGACCGCGCACGCCGAAGGGGGGTGCCCCCCCTTCTCGAACCCTC | |
| | CCGGCCCGCTAACGCGGGCCTCCCATCCCCCCAGGGGCTGCGCCCCTC | |
| | GGCCGCGAACGGCCTCACCCCAAAAATGGCAGCGCTGGCCAATTCCCG | |
| | AGGCACGAACCCAGTGGACATAAGCCTGTTCGGTTCGTAAGCTGTAAT | |
| | GCAAGTAGCGTATGCGCTCACGCAACTGGTCCAGAACCTTGACCGAAC | |
| | GCAGCGGTGGTAACGGCGCAGTGGCGGTTTTCATGGCTTGTTATGACT | |
| | GTTTTTTTGGGGTACAGTCTATGCCTCGGGCATCCAAGCAGCAAGCGC | |
| | GTTACGCCGTGGGTCGATGTTTGATGTTATGGAGCAGCAACGATGTTA | |
| | CGCAGCAGGGCAGTCGCCCTAAAACAAAGTTAAACATCATGGGGGAAG | |
| | CGGTGATCGCCGAAGTATCGACTCAACTATCAGAGGTAGTTGGCGTCA | |
| | TCGAGCGCCATCTCGAACCGACGTTGCTGGCCGTACATTTGTACGGCT | |
| | CCGCAGTGGATGGCGGCCTGAAGCCACACAGCGATATTGATTTGCTGG | |
| | TTACGGTGACCGTAAGGCTTGATGAAACAACGCGGCGAGCTTTGATCA | |
| | ACGACCTTTTGGAAACTTCGGCTTCCCCTGGAGAGAGCGAGATTCTCC | |
| | GCGCTGTAGAAGTCACCATTGTTGTGCACGACGACATCATTCCGTGGC | |
| | GTTATCCAGCTAAGCGCGAACTGCAATTTGGAGAATGGCAGCGCAATG | |
| | ACATTCTTGCAGGTATCTTCGAGCCAGCCACGATCGACATTGATCTGG | |
| | CTATCTTGCTGACAAAAGCAAGAGAACATAGCGTTGCCTTGGTAGGTC | |
| | CAGCGGCGGAGGAACTCTTTGATCCGGTTCCTGAACAGGATCTATTTG | |
| | AGGCGCTAAATGAAACCTTAACGCTATGGAACTCGCCGCCCGACTGGG | |
| | CTGGCGATGAGCGAAATGTAGTGCTTACGTTGTCCCGCATTTGGTACA | |
| | GCGCAGTAACCGGCAAAATCGCGCCGAAGGATGTCGCTGCCGACTGGG | |
| | CAATGGAGCGCCTGCCGGCCCAGTATCAGCCCGTCATACTTGAAGCTA | |
| | GACAGGCTTATCTTGGACAAGAAGAAGATCGCTTGGCCTCGCGCGCAG | |
| | ATCAGTTGGAAGAATTTGTCCATTACGTGAAAGGCGAGATCACCAAGG | |
| | TAGTCGGCAAATAATGTCTAGCTAGAAATTCGTTCAAGCCGACGCCGC | |
| | TTCGCGGCGCGGCTTAACTCAAGCGTTAGATGCACTAAGCACATAATT | |
| | GCTCACAGCCAAACTATCAGGTCAAGTCTGCTTTTATTATTTTTAAGC | |
| | GTGCATAATAAGCCCTACACAAATTGGGAGATATATCATGCTGTCAGA | |
| | CCAAGTTTACTCATATATACTTTAGATTGATTTAAAACTTCATTTTTA | |
| | ATTTAAAAGGATCTAGGTGAAGATCCTTTTTGATAATCTCATGACCAA | |
| | AATCCCTTAACGTGAGTTTTCGTTCCACTGAGCGTCAGACCCCGTAGA | |
| | AAAGATCAAAGGATCTTCTTGAGATCCTTTTTTTCTGCGCGTAATCTG | |
| | CTGCTTGCAAACAAAAAAACCACCGCTACCAGCGGTGGTTTGTTTGCC | |
| | GGATCAAGAGCTACCAACTCTTTTTCCGAAGGTAACTGGCTTCAGCAG | |
| | AGCGCAGATACCAAATACTGTCCTTCTAGTGTAGCCGTAGTTAGGCCA | |
| | CCACTTCAAGAACTCTGTAGCACCGCCTACATACCTCGCTCTGCTAAT | |
| | CCTGTTACCAGTGGCTGCTGCCAGTGGCGATAAGTCGTGTCTTACCGG | |
| | GTTGGACTCAAGACGATAGTTACCGGATAAGGCGCAGCGGTCGGGCTG | |
| | AACGGGGGGTTCGTGCACACAGCCCAGCTTGGAGCGAACGACCTACAC | |
| | CGAACTGAGATACCTACAGCGTGAGCTATGAGAAAGCGCCACGCTTCC | |
| | CGAAGGGAGAAAGGCGGACAGGTATCCGGTAAGCGGCAGGGTCGGAAC | |
| | AGGAGAGCGCACGAGGGAGCTTCCAGGGGGAAACGCCTGGTATCTTTA | |
| | TAGTCCTGTCGGGTTTCGCCACCTCTGACTTGAGCGTCGATTTTTGTG | |
| | ATGCTCGTCAGGGGGGCGGAGCCTATGGAAAAACGCCAGCAACGCGGC | |
| | CTTTTTACGGTTCCTGGCAGATCCTAGATGTGGCGCAACGATGCCGGC | |
| | GACAAGCAGGAGCGCACCGACTTCTTCCGCATCAAGTGTTTTGGCTCT | |
| | CAGGCCGAGGCCCACGGCAAGTATTTGGGCAAGGGGTCGCTGGTATTC | |
| | GTGCAGGGCAAGATTCGGAATACCAAGTACGAGAAGGACGGCCAGACG | |
| | GTCTACGGGACCGACTTCATTGCCGATAAGGTGGATTATCTGGACACC | |
| | AAGGCACCAGGCGGGTCAAATCAGGAATAAGGGCACATTGCCCCGGCG | |
| | TGAGTCGGGGCAATCCCGCAAGGAGGGTGAATGAATCGGACGTTTGAC | |
| | CGGAAGGCATACAGGCAAGAACTGATCGACGCGGGGTTTTCCGCCGAG | |
| | GATGCCGAAACCATCGCAAGCCGCACCGTCATGCGTGCGCCCCGCGAA | |
| | ACCTTCCAGTCCGTCGGCTCGATGGTCCAGCAAGCTACGGCCAAGATC | |
| | GAGCGCGACAGCGTGCAACTGGCTCCCCCTGCCCTGCCCGCGCCATCG | |
| | GCCGCCGTGGAGCGTTCGCGTCGTCTTGAACAGGAGGCGGCAGGTTTG | |
| | GCGAAGTCGATGACCATCGACACGCGAGGAACTATGACGACCAAGAAG | |
| | CGAAAAACCGCCGGCGAGGACCTGGCAAAACAGGTCAGCGAGGCCAAG | |
| | CAGGCCGCGTTGCTGAAACACACGAAGCAGCAGATCAAGGAAATGCAG | |
| | CTTTCCTTGTTCGATATTGCGCCGTGGCCGGACACGATGCGAGCGATG | |
| | CCAAACGACACGGCCCGCTCTGCCCTGTTCACCACGCGCAACAAGAAA | |
| | ATCCCGCGCGAGGCGCTGCAAAACAAGGTCATTTTCCACGTCAACAAG | |
| | GACGTGAAGATCACCTACACCGGCGTCGAGCTGCGGGCCGACGATGAC | |
| | GAACTGGTGTGGCAGCAGGTGTTGGAGTACGCGAAGCGCACCCCTATC | |
| | GGCGAGCCGATCACCTTCACGTTCTACGAGCTTTGCCAGGACCTGGGC | |
| | TGGTCGATCAATGGCCGGTATTACACGAAGGCCGAGGAATGCCTGTCG | |
| | CGCCTACAGGCGACGGCGATGGGCTTCACGTCCGACCGCGTTGGGCAC | |
| | CTGGAATCGGTGTCGCTGCTGCACCGCTTCCGCGTCCTGGACCGTGGC | |
| | AAGAAAACGTCCCGTTGCCAGGTCCTGATCGACGAGGAAATCGTCGTG | |
| | CTGTTTGCTGGCGACCACTACACGAAATTCATATGGGAGAAGTACCGC | |
| | AAGCTGTCGCCGACGGCCCGACGGATGTTCGACTATTTCAGCTCGCAC | |
| | CGGGAGCCGTACCCGCTCAAGCTGGAAACCTTCCGCCTCATGTGCGGA | |
| | TCGGATTCCACCCGCGTGAAGAAGTGGCGCGAGCAGGTCGGCGAAGCC | |
| | TGCGAAGAGTTGCGAGGCAGCGGCCTGGTGGAACACGCCTGGGTCAAT | |
| | GATGACCTGGTGCATTGCAAACGCTAGGGCCTTGTGGGGTCAGTTCCG | |
| | GCTGGGGGTTCAGCAGCCCCTGCTCGGATCTGTTGGACCGGACAGTAG | |
| | TCATGGTTGATGGGCTGCCTGTATCGAGTGGTGATTTTGTGCCGAGCT | |
| | GCCGGTCGGGGAGCTGTTGGCTGGCTGGTGGCAGGATATATTGTGGTG | |
| | TAAACAAATTGACGCTTAGACAACTTAATAACACATTGCGGACGTTTT | |
| | TAATGTACTGGGGTTGAATGACAACCAACTCGGTCCATTTGCACCCCT | |
| | AATCATAATAGCTTTAATATTTCAAGATATTATTAAGTTAACGTTGTC | |
| | AATATCCTGGAAATTTTGCAAAATGAATCAAGCCTATATGGCTGTAAT | |
| | ATGAATTTAAAAGCAGCTCGATGTGGTGGTAATATGTAATTTACTTGA | |
| | TTCTAAAAAAATATCCCAAGTATTAATAATTTCTGCTAGGAAGAAGGT | |
| | TAGCTACGATTTACAGCAAAGCCAGAATACAAAGAACCATAAAGTGAT | |
| | TGAAGCTCGAAATATACGAAGGAACAAATATTTTTAAAAAAATACGCA | |
| | ATGACTTGGAACAAAAGAAAGTGATATATTTTTTGTTCTTAAACAAGC | |
| | ATCCCCTCTAAAGAATGGCAGTTTTCCTTTGCATGTAACTATTATGCT | |
| | CCCTTCGTTACAAAAATTTTGGACTACTATTGGGAACTTCTTCTGAAA | |
| | ATAGTTACTACCACTCTGTGGGTCTCATGCCGAATTCGGATCCGGAGG | |
| | TCAACATGGTGGAGCACGACACTCTGGTCTACTCCAAAAATGTCAAAG | |
| | ATACAGTCTCAGAAGATCAAAGGGCTATTGAGACTTTTCAACAAAGGA | |
| | TAATTTCGGGAAACCTCCTCGGATTCCATTGCCCAGCTATCTGTCACT | |
| | TCATCGAAAGGACAGTAGAAAAGGAAGGTGGCTCCTACAAATGCCATC | |
| | ATTGCGATAAAGGAAAGGCTATCATTCAAGATCTCTCTGCCGACAGTG | |
| | GTCCCAAAGATGGACCCCCACCCACGAGGAGCATCGTGGAAAAAGAAG | |
| | AGGTTCCAACCACGTCTACAAAGCAAGTGGATTGATGTGACATCTCCA | |
| | CTGACGTAAGGGATGACGCACAATCCCACTATCCTTCGCAAGACCCTT | |
| | CCTCTATATAAGGAAGTTCATTTCATTTGGAGAGGACACGCTCGAGTA | |
| | TAAGAGCTCATTTTTACAACAATTACCAACAACAACAAACAACAAACA | |
| | ACATTACAATTACATTTACAATTATCGATACAATGGTTGAACAAGATG | |
| | GATTGCACGCAGGTTCTCCGGCCGCTTGGGTGGAGAGGCTATTCGGCT | |
| | ATGACTGGGCACAACAGACAATCGGCTGCTCTGATGCCGCCGTGTTCC | |
| | GGCTGTCAGCGCAGGGGCGCCCGGTTCTTTTTGTCAAGACCGACCTGT | |
| | CCGGTGCCCTGAATGAACTGCAGGACGAGGCAGCGCGGCTATCGTGGC | |
| | TGGCCACGACGGGCGTTCCTTGCGCAGCTGTGCTCGACGTTGTCACTG | |
| | AAGCGGGAAGGGACTGGCTGCTATTGGGCGAAGTGCCGGGGCAGGATC | |
| | TCCTGTCATCTCACCTTGCTCCTGCCGAGAAAGTATCCATCATGGCTG | |
| | ATGCAATGCGGCGGCTGCATACGCTTGATCCGGCTACCTGCCCATTCG | |
| | ACCACCAAGCGAAACATCGCATCGAGCGAGCACGTACTCGGATGGAAG | |
| | CCGGTCTTGTCGATCAGGATGATCTGGACGAAGAGCATCAGGGGCTCG | |
| | CGCCAGCCGAACTGTTCGCCAGGCTCAAGGCGCGCATGCCCGACGGCG | |
| | AGGATCTCGTCGTGACTCATGGCGATGCCTGCTTGCCGAATATCATGG | |
| | TGGAAAATGGCCGCTTTTCTGGATTCATCGACTGTGGCCGGCTGGGTG | |
| | TGGCGGACCGCTATCAGGACATAGCGTTGGCTACCCGTGATATTGCTG | |
| | AAGAGCTTGGCGGCGAATGGGCTGACCGCTTCCTCGTGCTTTACGGTA | |
| | TCGCCGCTCCCGATTCGCAGCGCATCGCCTTCTATCGCCTTCTTGACG | |
| | AGTTCTTCTGAGCTTCTCTAGCTAGAGTCGATCGACAAGCTCGAGTTT | |
| | CTCCATAATAATGTGTGAGTAGTTCCCAGATAAGGGAATTAGGGTTCC | |
| | TATAGGGTTTCGCTCATGTGTTGAGCATATAAGAAACCCTTAGTATGT | |
| | ATTTGTATTTGTAAAATACTTCTATCAATAAAATTTCTAATTCCTAAA | |
| | ACCAAAATCCAGTACTAAAATCCAGATCGCTGCAAGAATTCAAGCTTG | |
| | GAGCCATGCCTCACATGTTAATGTACTACCAATGGAGGGCTGTACACA | |
| | TTTATGATTACGAAATTTTTTAATATATTTTATAGATTTCTTATGCAT | |
| | CATACAAAAATACATAATTATTCGTAACATTTTGGAGATACATATTCA | |
| | GATGCATCAAATTCTAATTAAACGTTAAAATATTTTGCAGACGTATCT | |
| | TCGTAACAATTTAAAACCTATACTATACATCACATTCGAAGGTCATTT | |
| | TATAATTTAAAATATTATGGAGATGCATCTTCGTTTATGTTTGCTCAG | |
| | ATGAAGATTTAAACCTTACAAACAATATGTAAAAAATGACGTACATAA | |
| | ATTCAGATAGTCCAAAAGTGTCATATATAAATAAAGATCAATAAGTGT | |
| | CAAAAAAAGTCAAGAACAACGATAGAGTAGCATAATGTCAAAATAAAA | |
| | TAAAATCCATGACACTACTACTATTATATATTAATGCACTAATGTGTA | |
| | TGTCTAACTACATCGTCTGTGCCTCCTCTGTCAGTTATGTCTCGTAAG | |
| | CCATCAATCCCCCGTCCTCCGGCGTTGTCTCCGGTATATCAATGTCCC | |
| | ATGTGCCTACGTCATGATGGCATCTAGGACATGTCTCACATCAGACAA | |
| | TTAGGAAAGATACATTTGCCAATGTATATCTGCGCAATCTCCACAATG | |
| | CAACGACATATAGGCAAGACATCCTCAACATAATTTAGTTGTGCATGC | |
| | TTCTCCTCGCTACTAGAATTCGAGCTCGGAGGTCAACATGGTGGAGCA | |
| | CGACACTCTGGTCTACTCCAAAAATGTCAAAGATACAGTCTCAGAAGA | |
| | TCAAAGGGCTATTGAGACTTTTCAACAAAGGATAATTTCGGGAAACCT | |
| | CCTCGGATTCCATTGCCCAGCTATCTGTCACTTCATCGAAAGGACAGT | |
| | AGAAAAGGAAGGTGGCTCCTACAAATGCCATCATTGCGATAAAGGAAA | |
| | GGCTATCATTCAAGATCTCTCTGCCGACAGTGGTCCCAAAGATGGACC | |
| | CCCACCCACGAGGAGCATCGTGGAAAAAGAAGAGGTTCCAACCACGTC | |
| | TACAAAGCAAGTGGATTGATGTGATAACATGGTGGAGCACGACACTCT | |
| | GGTCTACTCCAAAAATGTCAAAGATACAGTCTCAGAAGATCAAAGGGC | |
| | TATTGAGACTTTTCAACAAAGGATAATTTCGGGAAACCTCCTCGGATT | |
| | CCATTGCCCAGCTATCTGTCACTTCATCGAAAGGACAGTAGAAAAGGA | |
| | AGGTGGCTCCTACAAATGCCATCATTGCGATAAAGGAAAGGCTATCAT | |
| | TCAAGATCTCTCTGCCGACAGTGGTCCCAAAGATGGACCCCCACCCAC | |
| | GAGGAGCATCGTGGAAAAAGAAGAGGTTCCAACCACGTCTACAAAGCA | |
| | AGTGGATTGATGTGACATCTCCACTGACGTAAGGGATGACGCACAATC | |
| | CCACTATCCTTCGCAAGACCCTTCCTCTATATAAGGAAGTTCATTTCA | |
| | TTTGGAGAGGACACGCTCGAGTATAAGAGCTCATTTTTACAACAATTA | |
| | CCAACAACAACAAACAACAAACAACATTACAATTACATTTACAATTAT | |
| | CGATACAATGGATCATGCGACCCTCGCCATGATCCTCGCGATCTGGTT | |
| | CATCAGCTTCCACTTCATCAAGCTGCTGTTCTCCCAGCAGACCACCAA | |
| | GCTGCTTCCGCCAGGACCAAAGCCGCTTCCGATCATCGGCAACATCCT | |
| | TGAGGTGGGCAAGAAGCCGCATCGGTCCTTCGCCAACCTCGCCAAGAT | |
| | TCACGGCCCACTCATTTCCCTCAGACTCGGCTCTGTGACCACCATCGT | |
| | TGTGTCCTCTGCCGACGTGGCCAAAGAGATGTTCCTCAAGAAGGATCA | |
| | CCCGCTCTCCAACCGCACGATCCCGAATAGTGTTACAGCCGGCGACCA | |
| | CCACAAGCTCACCATGTCTTGGCTCCCGGTGTCTCCGAAGTGGCGCAA | |
| | CTTCCGCAAGATTACCGCCGTGCATCTGCTCTCCCCACAGAGACTCGA | |
| | TGCCTGCCAGACATTCAGGCACGCCAAGGTGCAGCAGCTCTACGAGTA | |
| | CGTTCAAGAGTGCGCCCAGAAAGGCCAGGCCGTGGATATTGGCAAGGC | |
| | CGCCTTTACGACCAGCCTCAACCTCCTCAGCAAGCTGTTCTTCAGCGT | |
| | CGAGCTGGCGCACCACAAGTCCCATACCAGCCAAGAGTTCAAAGAGCT | |
| | GATCTGGAACATCATGGAAGATATAGGCAAGCCGAACTACGCCGACTA | |
| | CTTCCCGATTCTCGGCTGCGTTGACCCATCTGGCATTAGAAGAAGGCT | |
| | CGCCTGCTCCTTCGACAAGCTGATCGCCGTGTTCCAGGGCATCATCTG | |
| | CGAGAGACTCGCCCCAGATTCCTCCACCACAACTACCACCACCACCGA | |
| | CGACGTGCTCGATGTGCTCCTCCAGCTGTTCAAGCAGAACGAGCTGAC | |
| | GATGGGCGAGATCAACCACCTCCTCGTGGACATCTTCGACGCCGGCAC | |
| | CGATACCACATCCTCCACATTCGAGTGGGTGATGACCGAGCTGATCCG | |
| | CAATCCAGAGATGATGGAAAAGGCCCAAGAGGAAATCAAGCAGGTCCT | |
| | CGGCAAGGACAAGCAGATCCAAGAGTCCGACATCATCAACCTGCCGTA | |
| | CCTCCAGGCGATCATCAAAGAGACACTCCGCCTCCATCCGCCGACCGT | |
| | GTTCTTGCTCCCAAGAAAGGCCGACACCGATGTCGAGCTGTACGGCTA | |
| | CATCGTGCCGAAGGATGCCCAGATCCTCGTGAACCTCTGGGCCATTGG | |
| | CAGGGACCCAAACGCCTGGCAGAACGCCGATATTTTCAGCCCAGAGCG | |
| | CTTCATCGGCTGCGAGATCGATGTTAAGGGCCGCGATTTCGGCCTCCT | |
| | TCCATTTGGCGCTGGCCGCAGAATTTGCCCAGGCATGAATCTCGCCAT | |
| | CAGGATGCTCACCCTCATGCTCGCCACACTCCTCCAGTTCTTCAACTG | |
| | GAAGCTCGAAGGCGACATCTCCCCGAAGGACCTCGACATGGACGAGAA | |
| | GTTCGGCATTGCGCTCCAAAAGACCAAGCCGCTCAAGCTCATCCCGAT | |
| | TCCGCGCTACGGTAGCGGAGCTACCAATTTTAGCCTCCTTAAGCAGGC | |
| | AGGTGATGTAGAAGAGAACCCCGGGCCTATGAAGATGATGAACGGCGA | |
| | GGACGCCAACGACCAGATGATCAAAGAGTCCTTCTTCATCACCCACGG | |
| | CAACCCGATCCTCACCGTCGAGGATACACATCCGCTCAGGCCGTTCTT | |
| | CGAGACATGGCGCGAGAAGATTTTCTCCAAGAAGCCGAAGGCCATCCT | |
| | CATCATCTCCGGCCACTGGGAGACAGTGAAGCCAACCGTGAACGCCGT | |
| | GCACATCAACGACACCATCCACGACTTCGACGACTACCCAGCCGCCAT | |
| | GTACCAGTTCAAGTACCCAGCTCCAGGCGAGCCAGAGCTTGCGAGAAA | |
| | GGTGGAAGAGATCCTCAAGAAGTCCGGGTTCGAGACAGCCGAGACAGA | |
| | CCAAAAGAGGGGCCTTGATCACGGCGCCTGGGTTCCACTCATGCTCAT | |
| | GTATCCAGAGGCGGACATCCCGGTGTGCCAGCTCTCAGTTCAGCCACA | |
| | TCTCGACGGCACCTACCACTACAATCTCGGCAGAGCCCTCGCGCCGCT | |
| | CAAGAATGATGGCGTGCTCATTATTGGCTCCGGCAGCGCCACACATCC | |
| | ACTCGATGAGACACCGCACTACTTCGATGGTGTTGCCCCTTGGGCCGC | |
| | TGCCTTCGATTCTTGGCTTAGGAAGGCCCTCATCAACGGCCGCTTCGA | |
| | GGAAGTGAACATCTACGAGAGCAAGGCCCCGAACTGGAAGCTCGCCCA | |
| | TCCATTTCCAGAGCACTTCTACCCGCTCCACGTTGTGCTCGGCGCTGC | |
| | TGGTGAAAAGTGGAAGGCCGAGCTGATCCACTCCTCCTGGGATCATGG | |
| | CACACTTTGCCACGGCTCCTACAAGTTCACCTCCGCCGGATCCGGAGC | |
| | AACCAACTTTAGCCTGCTCAAGCAAGCAGGAGATGTTGAGGAAAATCC | |
| | TGGCCCCATGACCGCCATCAAGATGAACACCAACGGCGAGGGCGAGAC | |
| | ACAGCACATCCTCATGATCCCGTTCATGGCGCAGGGCCACCTCAGGCC | |
| | ATTTCTCGAACTCGCCATGTTCCTCTACAAGCGCTCCCACGTGATCAT | |
| | CACCCTGCTCACAACTCCGCTCAATGCCGGCTTCCTCAGGCACCTCCT | |
| | TCACCACCATTCCTACTCCTCCAGCGGCATCAGGATCGTCGAGCTGCC | |
| | ATTCAACTCCACCAACCACGGACTCCCACCGGGCATCGAGAACACCGA | |
| | TAAGCTCACACTCCCGCTCGTGGTGTCCCTCTTCCATTCCACCATCAG | |
| | CCTCGATCCGCACCTCCGCGATTACATCTCCAGGCATTTCAGCCCAGC | |
| | CAGGCCACCACTCTGCGTGATCCATGATGTGTTCCTCGGCTGGGTTGA | |
| | CCAGGTGGCCAAGGATGTGGGCTCTACAGGCGTGGTGTTCACAACAGG | |
| | CGGCGCTTATGGCACATCCGCCTACGTGTCCATCTGGAACGATCTCCC | |
| | GCACCAGAACTACTCCGACGACCAAGAGTTCCCGCTGCCAGGCTTCCC | |
| | AGAGAACCATAAGTTCCGCAGGTCCCAGCTCCATCGGTTCCTCAGATA | |
| | TGCCGACGGCTCCGACGATTGGTCCAAGTATTTCCAGCCGCAGCTCCG | |
| | CCAGTCCATGAAGTCTTTTGGCTGGCTCTGCAACTCCGTGGAAGAGAT | |
| | CGAGACACTCGGCTTCTCCATCCTCCGCAACTACACCAAGCTGCCGAT | |
| | CTGGGGCATCGGCCCACTTATTGCTTCCCCAGTGCAGCACTCCTCCTC | |
| | CGACAACAATTCAACAGGCGCCGAGTTCGTGCAGTGGCTCAGCCTCAA | |
| | AGAGCCGGACTCCGTCCTCTACATCTCCTTCGGCTCCCAGAACACGAT | |
| | CAGCCCGACGCAGATGATGGAACTCGCTGCTGGCCTTGAGTCCTCCGA | |
| | GAAGCCATTCCTCTGGGTGATCAGAGCCCCGTTCGGCTTCGACATCAA | |
| | CGAAGAGATGCGCCCAGAGTGGCTGCCAGAGGGCTTTGAGGAACGCAT | |
| | GAAGGTGAAGAAACAGGGCAAGCTCGTGTACAAGCTCGGCCCGCAGCT | |
| | TGAGATCCTCAACCATGAATCCATCGGCGGCTTTCTCACCCACTGCGG | |
| | ATGGAACAGCATCCTTGAGTCTCTTCGCGAGGGCGTTCCGATGCTTGG | |
| | ATGGCCACTTGCTGCCGAGCAGGCCTACAACCTCAAGTACCTCGAAGA | |
| | TGAGATGGGCGTCGCGGTTGAGCTTGCTAGAGGCCTCGAAGGCGAGAT | |
| | CTCCAAAGAGAAGGTCAAGCGCATCGTCGAGATGATCCTTGAGCGCAA | |
| | CGAGGGCTCCAAAGGCTGGGAGATGAAGAATCGCGCCGTGGAAATGGG | |
| | CAAAAAGCTCAAGGACGCCGTGAACGAGGAAAAAGAGCTGAAGGGCTC | |
| | CTCCGTGAAGGCGATCGACGATTTCCTCGACGCCGTCATGCAGGCCAA | |
| | ACTTGAGCCAAGCCTCCAGTGATAGTGAGCTTGAAGTGACATCACAAA | |
| | GTTGAAGGTAATAAAGCCAAATTAATTAAGACATTTTCATAATGATGT | |
| | CAAGAATGCAAAGCAAATTGCATAACTGCCTTTATGCAAAACATTAAT | |
| | ATAATATAAATTATAAAGAACTGCGCTCTCTGCTTCTTATTTTCTTAG | |
| | CTTCATTTATTAGTCACTAGCTGTTCAGAATTTTCAGTATCTTTTGAT | |
| | ATTACTAAGAACCTAATCACACAATGTATATTCTTATGCAGGAAAAGC | |
| | AGAATGCTGAGCTAAAAGAAAGGCTTTTTCCATTTTCGAGAGACAATG | |
| | AGAAAAGAAGAAGAAGAAGAAGAAGAAGAAGAAGAAGAAGAAGAAAAG | |
| | AGTAAATAATAAAGCCCCACAGGAGGCGAAGTTCTTGTAGCTCCATGT | |
| | TATCTAAGTTATTGATATTGTTTGCCCTATATTTTATTTCTGTCATTG | |
| | TGTATGTTTTGTTCAGTTTCGATCTCCTTGCAAAATGCAGAGATTATG | |
| | AGATGAATAAACTAAGTTATATTATTATACGTGTTAATATTCTCCTCC | |
| | TCTCTCTAGCTAGCCTTTTGTTTTCTCTTTTTCTTATTTGATTTTCTT | |
| | TAAATCAATCCATTTTAGGAGAGGGCCAGGGAGTGATCCAGCAAAACA | |
| | TGAAGATTAGAAGAAACTTCCCTCTTTTTTTCCTGAAAACAATTTAAC | |
| | GTCGAGATTTATCTCTTTTTGTAATGGAATCATTTCTACAGTTATGAC | |
| | GGTAATATGAAGATGAAGATGAAATATTTGGTGTGTCAAATAAAAAGC | |
| | TTGTGTGCTTAAGTTTGTGTTTTTTTCTTGGCTTGTTGTGTTATGAAT | |
| | TTGTGGCTTTTTCTAATATTAAATGAATGTAAGATCTCATTATAATGA | |
| | ATAAACAAATGTTTCTATAATCCATTGTGAATGTTTTGTTGGATCTCT | |
| | TCTGCAGCATATAACTACTGTATGTGCTATGGTATGGACTATGGAATA | |
| | TGATTAAAGATAAGCGCTTTACGAATTCCCATGGGGAGAGGAAGAAGT | |
| | TCCCAATCTTGAAATATCGAAGGTTCGCCTTTCTGTTGTAATATTGCT | |
| | CAATCCTCTACTTTTGTGCCGCCATCTGTATCAAAGTTGCTTCCCTTA | |
| | TCTCCTTTGTCAAATCTAAATTTGTTAAAAGTTCTTTCTCATTTGTTA | |
| | CTTCATTTGTATGTACGTATCTTGTTCTTGGCTCTCCTATTTCCACCG | |
| | AAATTAAGGCCTCAGTACCATAAATAAGTGAGAATGGTGTTTCTCTTG | |
| | TGCTTATCTTCGTCGTTGTCCTGTAATCCCATACTAGCCCAAGTAGTA | |
| | CTTCAGGGAACCTGCCTTTTGATGATTCTAACCGTTTCTTTATATTGT | |
| | TGACAATAATTGTATTTGTCGATTATGGTTTCCCATTAGCTATGGGAT | |
| | GGCAGTGCGCCGAAATAATTTGTTTAATTTCCCAGATTTTGAGAAAAT | |
| | CGGTAACTTTTGAACCTACGAATTGTGGCCCATTATCGCGTACTATTT | |
| | TTCTTGGGACACAAAATCGACATATTATGTTCCTCCAAATAATATCAA | |
| | CAACCTCTTTCTCTCGATCTATTTAAAGGCACATGCTTCTACCCACAT | |
| | AGAAAAATAATCAGTTAAGATTAATAAAAGTCGTTCCTTTCCTGGTGC | |
| | TTTAGGTAGAGGACCTACAATACATAACCCACTTCATAAACGGCCACG | |
| | GTGCTATGATTGAATGAAGAAACACTGCTGGTTGATGCATATTGTTTG | |
| | CACCCCGTTGACATTTATCACACCTTCTAATAAAAATTTTCGCTTTGT | |
| | GTTCTATTTTTTGCAAATAATACCGCCCCTTTTAGCGTCTTTAAAATG | |
| | ACCTTCCTCCAACGTGATTGCAACAATGTTCTTTATGTACTTCTTGCA | |
| | TCACATATTCAGTTTGTGATAGACCAAGGCACCGTGCTAAGGGTCCCA | |
| | TGAACATTTCGATACAAATTTTCTTCTATTAAGCAATATTGAGCAGCT | |
| | TTCACTCGCAATAATTGGGATTTTTAATCGACGCTCAGAGAATTCGCA | |
| | TGCGGAGCCAGAAGGTAATTATCCAAGATGTAGCATCAAGAATCCAAT | |
| | GTTTACGGGAAAAACTATGGAAGTATTATGTAAGCTCAGCAAGAAGCA | |
| | GATCAATATGCGGCACATATGCAACCTATGTTCAAAAATGAAGAATGT | |
| | ACAGATACAAGATCCTATACTGCCAGAATACGAAGAAGAATACGTAGA | |
| | AATTGAAAAAGAAGAACCAGGCGAAGAAAAGAATCTTGATGACGTAAG | |
| | CACTGACGACAACAATGAAAAGAAGAAGATAAGGTCGGTGATTGTGAA | |
| | AGAGACATAGAGGACACATGTAAGGTGGAAAATGTAAGGGCGGAAAGT | |
| | AACCTTATCACAAAGGAATCTTATCCCCCACTACTTATCCTTTTATAT | |
| | TTTTCCGTGTCATTTTTGCCCTTGAGTTTTCCTATATAAGGAACCAAG | |
| | TTCGGCATTTGTGAAAACAAGAAAAAATTTGGTGTAAGCTATTTTCTT | |
| | TGAAGTACTGAGGATACAACTTCAGAGAAATTTGTAAGTTTGTAATGA | |
| | TATCACTATCCTCTTTTCATCCATCAAGCACAACTGCTACCGCTACCG | |
| | CAGCTGCCGCAACCACACACCCTCCACAGCAATGCCCCGCATTCTCTT | |
| | CACCACCTTCGCATCTATCCTTGCCGCTACGACACCCTCGTCAACATC | |
| | TTGTGGTTAGGTGTGGAGGGGGTGGCTCTGCATCGGAGTCCGTATTCA | |
| | ACAGAGATTCAGCCGCAACTAGGGTTTCAAACGATCACTTGGATGTAT | |
| | CTAAAAGGGATGTAAAACTCAAAATTGCTATCATAGGGTTCGGAAACT | |
| | TTGGGCAATTTCTCGCAAAGACTATGGCAAAACAAGGTCATAGAGTAC | |
| | TAGCATATTCTCGATCAGACTATTCCCGGGCGGCAAAAGAGATTGGAG | |
| | TTGAATATTTTACTGATGCAGATGACTTGTGTGAGGAACATCCTGAAG | |
| | TAATTCTTTTGTGTACATCTATACTGTCTACTGAGAAGGTGTTGCGGT | |
| | CATTACCTCTCCATAGATTGCGCAGATCAACCCTGTTCGCTGATGTTT | |
| | TGAGTGTGAAAGAGTTCCCTCGCTCTTTATTTCTGCAACTCTTGCCCA | |
| | AAGATTTTGATATACTCTGTACCCACCCTATGTTTGGGCCAGACAGCG | |
| | GGAAAGACGGGTGGGGCGGACTCCCATTCGTATTTGACAAAGTAAGGG | |
| | TGGGCAGCGACCAATCTCGGACGTCAAGAGCTGAGGCATTTTTAGATG | |
| | TTTTTAGAAACGCCGGGTGTAGAATGGTGGAAATGTCATGCGTAGATC | |
| | ATGACAAACATGCCGCAGGCTCACAATTTATTACACACATGATGGGAA | |
| | GGGTACTGGAAAAACTAGCACTTGAAAATACGCCAATTAACACAAAGG | |
| | GATACGAATCATTACTAAATCTAGTTGATAACACAGCACGCGACAGCT | |
| | TTGAATTGTTTTACGGCCTATTCCTTTATAACAAGAATGCTATGGAGC | |
| | AGTTGGATAGAATGGACTGGGCTTTTGAGATGGTGAAAAAGCAGCTAA | |
| | GCGGTTATTTGCATGATCTGGTGAGGAAACAACTAATGCTGGAAGGGA | |
| | ACAATGATCAGGCGGAGGTCACCTTCGATAAGCCCCTGATGCTACCCT | |
| | CTCCCACAATTAACCCACCTCAAATAGTGCCCTCCGCTGACATGGCTG | |
| | AGAAAAAGCACGATCTTGTAGTTGTCAATGGGACTAGGATGGCTTCTA | |
| | TGATATCCTCTTCAGCTGTGACTACAGTCAGCCGTGCTTCTACGGTGC | |
| | AATCGGCCGCGGTGGCTCCATTCGGCGGCCTCAAATCCATGACTGGAT | |
| | TCCCAGTTAAGAAGGTCAACACTGACATTACTTCCATTACAAGCAATG | |
| | GTGGAAGAGTAAAGTGCATGCAGATGAATTACCAGAACGATGACTTAA | |
| | GAATCAAAGAAATTAAAGAGTTGTTACCACCTGTGGCTCTTTTGGAAA | |
| | AATTCCCAGCAACTGAGAATGCTGCAAACACAGTTGCTCATGCAAGAA | |
| | AGGCTATTCACAAAATCTTGAAGGGTAATGATGACAGGTTACTTGTTG | |
| | TGATCGGACCATGCTCAATACATGATCCTGTTGCTGCAAAGGAATACG | |
| | CTACTAGATTGCTTGCATTGAGGGAAGAGTTAAAGGATGAACTTGAGA | |
| | TTGTTATGAGAGTGTACTTCGAGAAACCAAGGACCACTGTTGGTTGGA | |
| | AGGGACTTATCAATGATCCTCACATGGACAACTCCTTCCAAATTAATG | |
| | ATGGTTTGAGAATCGCTAGGAAACTTTTGCTTGATATTAACGACTCAG | |
| | GTTTGCCAGCTGCAGGAGAATTTTTAGATATGATCACACCTCAGTACT | |
| | TAGCTGACCTTATGTCATGGGGTGCTATAGGAGCAAGAACAACCGAAA | |
| | GTCAAGTTCATAGGGAGCAGGCTTCCGGTTTGTCTTGTCCAGTGGGAT | |
| | TCAAAAATGGTACTGATGGAACAATTAAGGTTGCTATAGACGCAATTA | |
| | ACGCTGCAGGTGCTCCTCATTGTTTTCTTTCTGTTACAAAATGGGGAC | |
| | ACTCAGCAATCGTGAATACCAGTGGTAACGGAGATTGCCATATTATCT | |
| | TGAGAGGTGGAAAAGAACCAAATTATTCAGCTAAGCACGTTGCAGAAG | |
| | TGAAAGAGGGTTTGAACAAGGCTGGATTACCTGCACAAGTTATGATCG | |
| | ATTTCTCTCATGCTAACTCCTCTAAGCAATTCAAGAAACAGATGGATG | |
| | TTTGTGCTGACGTGTGCCAACAGATCGCTGGTGGAGAAAAGGCTATTA | |
| | TTGGTGTTATGGTGGAAAGTCACTTAGTTGAGGGAAATCAATCATTAG | |
| | AAAGTGGAGAGCCTCTTGCTTACGGAAAATCTATTACCGATGCATGCA | |
| | TCGGTTGGGAAGATACTGACGCTCTTTTGAGACAGTTGGCTAACGCAG | |
| | TTAAGGCAAGAAGGGGTTGATGAGCTTGTCAAGCAGATCGTTCAAACA | |
| | TTTGGCAATAAAGTTTCTTAAGATTGAATCCTGTTGCCGGTCTTGCGA | |
| | TGATTATCATATAATTTCTGTTGAATTACGTTAAGCATGTAATAATTA | |
| | ACATGTAATGCATGACGTTATTTATGAGATGGGTTTTTATGATTAGAG | |
| | TCCCGCAATTATACATTTAATACGCGATAGAAAACAAAATATAGCGCG | |
| | CAAACTAGGATAAATTATCGCGCGCGGTGTCATCTATGTTACTAGATC | |
| | GA | |
| MAR -PL- | GTGATCCGTTTAAACTATCAGTGTTTGACAGGATATATTGGCGGGTAA | 163 |
| 1233 | ACCTAAGAGAAAAGAGCGTTTATTAGAATAATCGGATATTTAAAAGGG | |
| construct- | CGTGAAAAGGTTTATCCGTTCGTCCATTTGTATGTGCCAGCCGCCTTT | |
| MAR | GCGACGCTCACCGGGCTGGTTGCCCTCGCCGCTGGGCTGGCGGCCGTC | |
| | TATGGCCCTGCAAACGCGCCAGAAACGCCGTCGAAGCCGTGTGCGAGA | |
| | CACCGCGGCCGCCGGCGTTGTGGATACCTCGCGGAAAACTTGGCCCTC | |
| | ACTGACAGATGAGGGGCGGACGTTGACACTTGAGGGGCCGACTCACCC | |
| | GGCGCGGCGTTGACAGATGAGGGGCAGGCTCGATTTCGGCCGGCGACG | |
| | TGGAGCTGGCCAGCCTCGCAAATCGGCGAAAACGCCTGATTTTACGCG | |
| | AGTTTCCCACAGATGATGTGGACAAGCCTGGGGATAAGTGCCCTGCGG | |
| | TATTGACACTTGAGGGGCGCGACTACTGACAGATGAGGGGCGCGATCC | |
| | TTGACACTTGAGGGGCAGAGTGCTGACAGATGAGGGGCGCACCTATTG | |
| | ACATTTGAGGGGCTGTCCACAGGCAGAAAATCCAGCATTTGCAAGGGT | |
| | TTCCGCCCGTTTTTCGGCCACCGCTAACCTGTCTTTTAACCTGCTTTT | |
| | AAACCAATATTTATAAACCTTGTTTTTAACCAGGGCTGCGCCCTGTGC | |
| | GCGTGACCGCGCACGCCGAAGGGGGGTGCCCCCCCTTCTCGAACCCTC | |
| | CCGGCCCGCTAACGCGGGCCTCCCATCCCCCCAGGGGCTGCGCCCCTC | |
| | GGCCGCGAACGGCCTCACCCCAAAAATGGCAGCGCTGGCCAATTCCCG | |
| | AGGCACGAACCCAGTGGACATAAGCCTGTTCGGTTCGTAAGCTGTAAT | |
| | GCAAGTAGCGTATGCGCTCACGCAACTGGTCCAGAACCTTGACCGAAC | |
| | GCAGCGGTGGTAACGGCGCAGTGGCGGTTTTCATGGCTTGTTATGACT | |
| | GTTTTTTTGGGGTACAGTCTATGCCTCGGGCATCCAAGCAGCAAGCGC | |
| | GTTACGCCGTGGGTCGATGTTTGATGTTATGGAGCAGCAACGATGTTA | |
| | CGCAGCAGGGCAGTCGCCCTAAAACAAAGTTAAACATCATGGGGGAAG | |
| | CGGTGATCGCCGAAGTATCGACTCAACTATCAGAGGTAGTTGGCGTCA | |
| | TCGAGCGCCATCTCGAACCGACGTTGCTGGCCGTACATTTGTACGGCT | |
| | CCGCAGTGGATGGCGGCCTGAAGCCACACAGCGATATTGATTTGCTGG | |
| | TTACGGTGACCGTAAGGCTTGATGAAACAACGCGGCGAGCTTTGATCA | |
| | ACGACCTTTTGGAAACTTCGGCTTCCCCTGGAGAGAGCGAGATTCTCC | |
| | GCGCTGTAGAAGTCACCATTGTTGTGCACGACGACATCATTCCGTGGC | |
| | GTTATCCAGCTAAGCGCGAACTGCAATTTGGAGAATGGCAGCGCAATG | |
| | ACATTCTTGCAGGTATCTTCGAGCCAGCCACGATCGACATTGATCTGG | |
| | CTATCTTGCTGACAAAAGCAAGAGAACATAGCGTTGCCTTGGTAGGTC | |
| | CAGCGGCGGAGGAACTCTTTGATCCGGTTCCTGAACAGGATCTATTTG | |
| | AGGCGCTAAATGAAACCTTAACGCTATGGAACTCGCCGCCCGACTGGG | |
| | CTGGCGATGAGCGAAATGTAGTGCTTACGTTGTCCCGCATTTGGTACA | |
| | GCGCAGTAACCGGCAAAATCGCGCCGAAGGATGTCGCTGCCGACTGGG | |
| | CAATGGAGCGCCTGCCGGCCCAGTATCAGCCCGTCATACTTGAAGCTA | |
| | GACAGGCTTATCTTGGACAAGAAGAAGATCGCTTGGCCTCGCGCGCAG | |
| | ATCAGTTGGAAGAATTTGTCCATTACGTGAAAGGCGAGATCACCAAGG | |
| | TAGTCGGCAAATAATGTCTAGCTAGAAATTCGTTCAAGCCGACGCCGC | |
| | TTCGCGGCGCGGCTTAACTCAAGCGTTAGATGCACTAAGCACATAATT | |
| | GCTCACAGCCAAACTATCAGGTCAAGTCTGCTTTTATTATTTTTAAGC | |
| | GTGCATAATAAGCCCTACACAAATTGGGAGATATATCATGCTGTCAGA | |
| | CCAAGTTTACTCATATATACTTTAGATTGATTTAAAACTTCATTTTTA | |
| | ATTTAAAAGGATCTAGGTGAAGATCCTTTTTGATAATCTCATGACCAA | |
| | AATCCCTTAACGTGAGTTTTCGTTCCACTGAGCGTCAGACCCCGTAGA | |
| | AAAGATCAAAGGATCTTCTTGAGATCCTTTTTTTCTGCGCGTAATCTG | |
| | CTGCTTGCAAACAAAAAAACCACCGCTACCAGCGGTGGTTTGTTTGCC | |
| | GGATCAAGAGCTACCAACTCTTTTTCCGAAGGTAACTGGCTTCAGCAG | |
| | AGCGCAGATACCAAATACTGTCCTTCTAGTGTAGCCGTAGTTAGGCCA | |
| | CCACTTCAAGAACTCTGTAGCACCGCCTACATACCTCGCTCTGCTAAT | |
| | CCTGTTACCAGTGGCTGCTGCCAGTGGCGATAAGTCGTGTCTTACCGG | |
| | GTTGGACTCAAGACGATAGTTACCGGATAAGGCGCAGCGGTCGGGCTG | |
| | AACGGGGGGTTCGTGCACACAGCCCAGCTTGGAGCGAACGACCTACAC | |
| | CGAACTGAGATACCTACAGCGTGAGCTATGAGAAAGCGCCACGCTTCC | |
| | CGAAGGGAGAAAGGCGGACAGGTATCCGGTAAGCGGCAGGGTCGGAAC | |
| | AGGAGAGCGCACGAGGGAGCTTCCAGGGGGAAACGCCTGGTATCTTTA | |
| | TAGTCCTGTCGGGTTTCGCCACCTCTGACTTGAGCGTCGATTTTTGTG | |
| | ATGCTCGTCAGGGGGGCGGAGCCTATGGAAAAACGCCAGCAACGCGGC | |
| | CTTTTTACGGTTCCTGGCAGATCCTAGATGTGGCGCAACGATGCCGGC | |
| | GACAAGCAGGAGCGCACCGACTTCTTCCGCATCAAGTGTTTTGGCTCT | |
| | CAGGCCGAGGCCCACGGCAAGTATTTGGGCAAGGGGTCGCTGGTATTC | |
| | GTGCAGGGCAAGATTCGGAATACCAAGTACGAGAAGGACGGCCAGACG | |
| | GTCTACGGGACCGACTTCATTGCCGATAAGGTGGATTATCTGGACACC | |
| | AAGGCACCAGGCGGGTCAAATCAGGAATAAGGGCACATTGCCCCGGCG | |
| | TGAGTCGGGGCAATCCCGCAAGGAGGGTGAATGAATCGGACGTTTGAC | |
| | CGGAAGGCATACAGGCAAGAACTGATCGACGCGGGGTTTTCCGCCGAG | |
| | GATGCCGAAACCATCGCAAGCCGCACCGTCATGCGTGCGCCCCGCGAA | |
| | ACCTTCCAGTCCGTCGGCTCGATGGTCCAGCAAGCTACGGCCAAGATC | |
| | GAGCGCGACAGCGTGCAACTGGCTCCCCCTGCCCTGCCCGCGCCATCG | |
| | GCCGCCGTGGAGCGTTCGCGTCGTCTTGAACAGGAGGCGGCAGGTTTG | |
| | GCGAAGTCGATGACCATCGACACGCGAGGAACTATGACGACCAAGAAG | |
| | CGAAAAACCGCCGGCGAGGACCTGGCAAAACAGGTCAGCGAGGCCAAG | |
| | CAGGCCGCGTTGCTGAAACACACGAAGCAGCAGATCAAGGAAATGCAG | |
| | CTTTCCTTGTTCGATATTGCGCCGTGGCCGGACACGATGCGAGCGATG | |
| | CCAAACGACACGGCCCGCTCTGCCCTGTTCACCACGCGCAACAAGAAA | |
| | ATCCCGCGCGAGGCGCTGCAAAACAAGGTCATTTTCCACGTCAACAAG | |
| | GACGTGAAGATCACCTACACCGGCGTCGAGCTGCGGGCCGACGATGAC | |
| | GAACTGGTGTGGCAGCAGGTGTTGGAGTACGCGAAGCGCACCCCTATC | |
| | GGCGAGCCGATCACCTTCACGTTCTACGAGCTTTGCCAGGACCTGGGC | |
| | TGGTCGATCAATGGCCGGTATTACACGAAGGCCGAGGAATGCCTGTCG | |
| | CGCCTACAGGCGACGGCGATGGGCTTCACGTCCGACCGCGTTGGGCAC | |
| | CTGGAATCGGTGTCGCTGCTGCACCGCTTCCGCGTCCTGGACCGTGGC | |
| | AAGAAAACGTCCCGTTGCCAGGTCCTGATCGACGAGGAAATCGTCGTG | |
| | CTGTTTGCTGGCGACCACTACACGAAATTCATATGGGAGAAGTACCGC | |
| | AAGCTGTCGCCGACGGCCCGACGGATGTTCGACTATTTCAGCTCGCAC | |
| | CGGGAGCCGTACCCGCTCAAGCTGGAAACCTTCCGCCTCATGTGCGGA | |
| | TCGGATTCCACCCGCGTGAAGAAGTGGCGCGAGCAGGTCGGCGAAGCC | |
| | TGCGAAGAGTTGCGAGGCAGCGGCCTGGTGGAACACGCCTGGGTCAAT | |
| | GATGACCTGGTGCATTGCAAACGCTAGGGCCTTGTGGGGTCAGTTCCG | |
| | GCTGGGGGTTCAGCAGCCCCTGCTCGGATCTGTTGGACCGGACAGTAG | |
| | TCATGGTTGATGGGCTGCCTGTATCGAGTGGTGATTTTGTGCCGAGCT | |
| | GCCGGTCGGGGAGCTGTTGGCTGGCTGGTGGCAGGATATATTGTGGTG | |
| | TAAACAAATTGACGCTTAGACAACTTAATAACACATTGCGGACGTTTT | |
| | TAATGTACTGGGGTTGAATGACAACCAACTCGGTCCATTTGCACCCCT | |
| | AATCATAATAGCTTTAATATTTCAAGATATTATTAAGTTAACGTTGTC | |
| | AATATCCTGGAAATTTTGCAAAATGAATCAAGCCTATATGGCTGTAAT | |
| | ATGAATTTAAAAGCAGCTCGATGTGGTGGTAATATGTAATTTACTTGA | |
| | TTCTAAAAAAATATCCCAAGTATTAATAATTTCTGCTAGGAAGAAGGT | |
| | TAGCTACGATTTACAGCAAAGCCAGAATACAAAGAACCATAAAGTGAT | |
| | TGAAGCTCGAAATATACGAAGGAACAAATATTTTTAAAAAAATACGCA | |
| | ATGACTTGGAACAAAAGAAAGTGATATATTTTTTGTTCTTAAACAAGC | |
| | ATCCCCTCTAAAGAATGGCAGTTTTCCTTTGCATGTAACTATTATGCT | |
| | CCCTTCGTTACAAAAATTTTGGACTACTATTGGGAACTTCTTCTGAAA | |
| | ATAGTTACTACCACTCTGTGGGTCTCATGCCGAATTCGGATCCGGAGG | |
| | TCAACATGGTGGAGCACGACACTCTGGTCTACTCCAAAAATGTCAAAG | |
| | ATACAGTCTCAGAAGATCAAAGGGCTATTGAGACTTTTCAACAAAGGA | |
| | TAATTTCGGGAAACCTCCTCGGATTCCATTGCCCAGCTATCTGTCACT | |
| | TCATCGAAAGGACAGTAGAAAAGGAAGGTGGCTCCTACAAATGCCATC | |
| | ATTGCGATAAAGGAAAGGCTATCATTCAAGATCTCTCTGCCGACAGTG | |
| | GTCCCAAAGATGGACCCCCACCCACGAGGAGCATCGTGGAAAAAGAAG | |
| | AGGTTCCAACCACGTCTACAAAGCAAGTGGATTGATGTGACATCTCCA | |
| | CTGACGTAAGGGATGACGCACAATCCCACTATCCTTCGCAAGACCCTT | |
| | CCTCTATATAAGGAAGTTCATTTCATTTGGAGAGGACACGCTCGAGTA | |
| | TAAGAGCTCATTTTTACAACAATTACCAACAACAACAAACAACAAACA | |
| | ACATTACAATTACATTTACAATTATCGATACAATGGTTGAACAAGATG | |
| | GATTGCACGCAGGTTCTCCGGCCGCTTGGGTGGAGAGGCTATTCGGCT | |
| | ATGACTGGGCACAACAGACAATCGGCTGCTCTGATGCCGCCGTGTTCC | |
| | GGCTGTCAGCGCAGGGGCGCCCGGTTCTTTTTGTCAAGACCGACCTGT | |
| | CCGGTGCCCTGAATGAACTGCAGGACGAGGCAGCGCGGCTATCGTGGC | |
| | TGGCCACGACGGGCGTTCCTTGCGCAGCTGTGCTCGACGTTGTCACTG | |
| | AAGCGGGAAGGGACTGGCTGCTATTGGGCGAAGTGCCGGGGCAGGATC | |
| | TCCTGTCATCTCACCTTGCTCCTGCCGAGAAAGTATCCATCATGGCTG | |
| | ATGCAATGCGGCGGCTGCATACGCTTGATCCGGCTACCTGCCCATTCG | |
| | ACCACCAAGCGAAACATCGCATCGAGCGAGCACGTACTCGGATGGAAG | |
| | CCGGTCTTGTCGATCAGGATGATCTGGACGAAGAGCATCAGGGGCTCG | |
| | CGCCAGCCGAACTGTTCGCCAGGCTCAAGGCGCGCATGCCCGACGGCG | |
| | AGGATCTCGTCGTGACTCATGGCGATGCCTGCTTGCCGAATATCATGG | |
| | TGGAAAATGGCCGCTTTTCTGGATTCATCGACTGTGGCCGGCTGGGTG | |
| | TGGCGGACCGCTATCAGGACATAGCGTTGGCTACCCGTGATATTGCTG | |
| | AAGAGCTTGGCGGCGAATGGGCTGACCGCTTCCTCGTGCTTTACGGTA | |
| | TCGCCGCTCCCGATTCGCAGCGCATCGCCTTCTATCGCCTTCTTGACG | |
| | AGTTCTTCTGAGCTTCTCTAGCTAGAGTCGATCGACAAGCTCGAGTTT | |
| | CTCCATAATAATGTGTGAGTAGTTCCCAGATAAGGGAATTAGGGTTCC | |
| | TATAGGGTTTCGCTCATGTGTTGAGCATATAAGAAACCCTTAGTATGT | |
| | ATTTGTATTTGTAAAATACTTCTATCAATAAAATTTCTAATTCCTAAA | |
| | ACCAAAATCCAGTACTAAAATCCAGATCGCTGCAAGAATTCAAGCTTG | |
| | GAGCCATGCCTCACATGTTAATGTACTACCAATGGAGGGCTGTACACA | |
| | TTTATGATTACGAAATTTTTTAATATATTTTATAGATTTCTTATGCAT | |
| | CATACAAAAATACATAATTATTCGTAACATTTTGGAGATACATATTCA | |
| | GATGCATCAAATTCTAATTAAACGTTAAAATATTTTGCAGACGTATCT | |
| | TCGTAACAATTTAAAACCTATACTATACATCACATTCGAAGGTCATTT | |
| | TATAATTTAAAATATTATGGAGATGCATCTTCGTTTATGTTTGCTCAG | |
| | ATGAAGATTTAAACCTTACAAACAATATGTAAAAAATGACGTACATAA | |
| | ATTCAGATAGTCCAAAAGTGTCATATATAAATAAAGATCAATAAGTGT | |
| | CAAAAAAAGTCAAGAACAACGATAGAGTAGCATAATGTCAAAATAAAA | |
| | TAAAATCCATGACACTACTACTATTATATATTAATGCACTAATGTGTA | |
| | TGTCTAACTACATCGTCTGTGCCTCCTCTGTCAGTTATGTCTCGTAAG | |
| | CCATCAATCCCCCGTCCTCCGGCGTTGTCTCCGGTATATCAATGTCCC | |
| | ATGTGCCTACGTCATGATGGCATCTAGGACATGTCTCACATCAGACAA | |
| | TTAGGAAAGATACATTTGCCAATGTATATCTGCGCAATCTCCACAATG | |
| | CAACGACATATAGGCAAGACATCCTCAACATAATTTAGTTGTGCATGC | |
| | TTCTCCTCGCTACTAGAATTCGAGCTCGGAGGTCAACATGGTGGAGCA | |
| | CGACACTCTGGTCTACTCCAAAAATGTCAAAGATACAGTCTCAGAAGA | |
| | TCAAAGGGCTATTGAGACTTTTCAACAAAGGATAATTTCGGGAAACCT | |
| | CCTCGGATTCCATTGCCCAGCTATCTGTCACTTCATCGAAAGGACAGT | |
| | AGAAAAGGAAGGTGGCTCCTACAAATGCCATCATTGCGATAAAGGAAA | |
| | GGCTATCATTCAAGATCTCTCTGCCGACAGTGGTCCCAAAGATGGACC | |
| | CCCACCCACGAGGAGCATCGTGGAAAAAGAAGAGGTTCCAACCACGTC | |
| | TACAAAGCAAGTGGATTGATGTGATAACATGGTGGAGCACGACACTCT | |
| | GGTCTACTCCAAAAATGTCAAAGATACAGTCTCAGAAGATCAAAGGGC | |
| | TATTGAGACTTTTCAACAAAGGATAATTTCGGGAAACCTCCTCGGATT | |
| | CCATTGCCCAGCTATCTGTCACTTCATCGAAAGGACAGTAGAAAAGGA | |
| | AGGTGGCTCCTACAAATGCCATCATTGCGATAAAGGAAAGGCTATCAT | |
| | TCAAGATCTCTCTGCCGACAGTGGTCCCAAAGATGGACCCCCACCCAC | |
| | GAGGAGCATCGTGGAAAAAGAAGAGGTTCCAACCACGTCTACAAAGCA | |
| | AGTGGATTGATGTGACATCTCCACTGACGTAAGGGATGACGCACAATC | |
| | CCACTATCCTTCGCAAGACCCTTCCTCTATATAAGGAAGTTCATTTCA | |
| | TTTGGAGAGGACACGCTCGAGTATAAGAGCTCATTTTTACAACAATTA | |
| | CCAACAACAACAAACAACAAACAACATTACAATTACATTTACAATTAT | |
| | CGATACAATGGATCATGCGACCCTCGCCATGATCCTCGCGATCTGGTT | |
| | CATCAGCTTCCACTTCATCAAGCTGCTGTTCTCCCAGCAGACCACCAA | |
| | GCTGCTTCCGCCAGGACCAAAGCCGCTTCCGATCATCGGCAACATCCT | |
| | TGAGGTGGGCAAGAAGCCGCATCGGTCCTTCGCCAACCTCGCCAAGAT | |
| | TCACGGCCCACTCATTTCCCTCAGACTCGGCTCTGTGACCACCATCGT | |
| | TGTGTCCTCTGCCGACGTGGCCAAAGAGATGTTCCTCAAGAAGGATCA | |
| | CCCGCTCTCCAACCGCACGATCCCGAATAGTGTTACAGCCGGCGACCA | |
| | CCACAAGCTCACCATGTCTTGGCTCCCGGTGTCTCCGAAGTGGCGCAA | |
| | CTTCCGCAAGATTACCGCCGTGCATCTGCTCTCCCCACAGAGACTCGA | |
| | TGCCTGCCAGACATTCAGGCACGCCAAGGTGCAGCAGCTCTACGAGTA | |
| | CGTTCAAGAGTGCGCCCAGAAAGGCCAGGCCGTGGATATTGGCAAGGC | |
| | CGCCTTTACGACCAGCCTCAACCTCCTCAGCAAGCTGTTCTTCAGCGT | |
| | CGAGCTGGCGCACCACAAGTCCCATACCAGCCAAGAGTTCAAAGAGCT | |
| | GATCTGGAACATCATGGAAGATATAGGCAAGCCGAACTACGCCGACTA | |
| | CTTCCCGATTCTCGGCTGCGTTGACCCATCTGGCATTAGAAGAAGGCT | |
| | CGCCTGCTCCTTCGACAAGCTGATCGCCGTGTTCCAGGGCATCATCTG | |
| | CGAGAGACTCGCCCCAGATTCCTCCACCACAACTACCACCACCACCGA | |
| | CGACGTGCTCGATGTGCTCCTCCAGCTGTTCAAGCAGAACGAGCTGAC | |
| | GATGGGCGAGATCAACCACCTCCTCGTGGACATCTTCGACGCCGGCAC | |
| | CGATACCACATCCTCCACATTCGAGTGGGTGATGACCGAGCTGATCCG | |
| | CAATCCAGAGATGATGGAAAAGGCCCAAGAGGAAATCAAGCAGGTCCT | |
| | CGGCAAGGACAAGCAGATCCAAGAGTCCGACATCATCAACCTGCCGTA | |
| | CCTCCAGGCGATCATCAAAGAGACACTCCGCCTCCATCCGCCGACCGT | |
| | GTTCTTGCTCCCAAGAAAGGCCGACACCGATGTCGAGCTGTACGGCTA | |
| | CATCGTGCCGAAGGATGCCCAGATCCTCGTGAACCTCTGGGCCATTGG | |
| | CAGGGACCCAAACGCCTGGCAGAACGCCGATATTTTCAGCCCAGAGCG | |
| | CTTCATCGGCTGCGAGATCGATGTTAAGGGCCGCGATTTCGGCCTCCT | |
| | TCCATTTGGCGCTGGCCGCAGAATTTGCCCAGGCATGAATCTCGCCAT | |
| | CAGGATGCTCACCCTCATGCTCGCCACACTCCTCCAGTTCTTCAACTG | |
| | GAAGCTCGAAGGCGACATCTCCCCGAAGGACCTCGACATGGACGAGAA | |
| | GTTCGGCATTGCGCTCCAAAAGACCAAGCCGCTCAAGCTCATCCCGAT | |
| | TCCGCGCTACGGTAGCGGAGCTACCAATTTTAGCCTCCTTAAGCAGGC | |
| | AGGTGATGTAGAAGAGAACCCCGGGCCTATGAAGATGATGAACGGCGA | |
| | GGACGCCAACGACCAGATGATCAAAGAGTCCTTCTTCATCACCCACGG | |
| | CAACCCGATCCTCACCGTCGAGGATACACATCCGCTCAGGCCGTTCTT | |
| | CGAGACATGGCGCGAGAAGATTTTCTCCAAGAAGCCGAAGGCCATCCT | |
| | CATCATCTCCGGCCACTGGGAGACAGTGAAGCCAACCGTGAACGCCGT | |
| | GCACATCAACGACACCATCCACGACTTCGACGACTACCCAGCCGCCAT | |
| | GTACCAGTTCAAGTACCCAGCTCCAGGCGAGCCAGAGCTTGCGAGAAA | |
| | GGTGGAAGAGATCCTCAAGAAGTCCGGGTTCGAGACAGCCGAGACAGA | |
| | CCAAAAGAGGGGCCTTGATCACGGCGCCTGGGTTCCACTCATGCTCAT | |
| | GTATCCAGAGGCGGACATCCCGGTGTGCCAGCTCTCAGTTCAGCCACA | |
| | TCTCGACGGCACCTACCACTACAATCTCGGCAGAGCCCTCGCGCCGCT | |
| | CAAGAATGATGGCGTGCTCATTATTGGCTCCGGCAGCGCCACACATCC | |
| | ACTCGATGAGACACCGCACTACTTCGATGGTGTTGCCCCTTGGGCCGC | |
| | TGCCTTCGATTCTTGGCTTAGGAAGGCCCTCATCAACGGCCGCTTCGA | |
| | GGAAGTGAACATCTACGAGAGCAAGGCCCCGAACTGGAAGCTCGCCCA | |
| | TCCATTTCCAGAGCACTTCTACCCGCTCCACGTTGTGCTCGGCGCTGC | |
| | TGGTGAAAAGTGGAAGGCCGAGCTGATCCACTCCTCCTGGGATCATGG | |
| | CACACTTTGCCACGGCTCCTACAAGTTCACCTCCGCCGGATCCGGAGC | |
| | AACCAACTTTAGCCTGCTCAAGCAAGCAGGAGATGTTGAGGAAAATCC | |
| | TGGCCCCATGACCGCCATCAAGATGAACACCAACGGCGAGGGCGAGAC | |
| | ACAGCACATCCTCATGATCCCGTTCATGGCGCAGGGCCACCTCAGGCC | |
| | ATTTCTCGAACTCGCCATGTTCCTCTACAAGCGCTCCCACGTGATCAT | |
| | CACCCTGCTCACAACTCCGCTCAATGCCGGCTTCCTCAGGCACCTCCT | |
| | TCACCACCATTCCTACTCCTCCAGCGGCATCAGGATCGTCGAGCTGCC | |
| | ATTCAACTCCACCAACCACGGACTCCCACCGGGCATCGAGAACACCGA | |
| | TAAGCTCACACTCCCGCTCGTGGTGTCCCTCTTCCATTCCACCATCAG | |
| | CCTCGATCCGCACCTCCGCGATTACATCTCCAGGCATTTCAGCCCAGC | |
| | CAGGCCACCACTCTGCGTGATCCATGATGTGTTCCTCGGCTGGGTTGA | |
| | CCAGGTGGCCAAGGATGTGGGCTCTACAGGCGTGGTGTTCACAACAGG | |
| | CGGCGCTTATGGCACATCCGCCTACGTGTCCATCTGGAACGATCTCCC | |
| | GCACCAGAACTACTCCGACGACCAAGAGTTCCCGCTGCCAGGCTTCCC | |
| | AGAGAACCATAAGTTCCGCAGGTCCCAGCTCCATCGGTTCCTCAGATA | |
| | TGCCGACGGCTCCGACGATTGGTCCAAGTATTTCCAGCCGCAGCTCCG | |
| | CCAGTCCATGAAGTCTTTTGGCTGGCTCTGCAACTCCGTGGAAGAGAT | |
| | CGAGACACTCGGCTTCTCCATCCTCCGCAACTACACCAAGCTGCCGAT | |
| | CTGGGGCATCGGCCCACTTATTGCTTCCCCAGTGCAGCACTCCTCCTC | |
| | CGACAACAATTCAACAGGCGCCGAGTTCGTGCAGTGGCTCAGCCTCAA | |
| | AGAGCCGGACTCCGTCCTCTACATCTCCTTCGGCTCCCAGAACACGAT | |
| | CAGCCCGACGCAGATGATGGAACTCGCTGCTGGCCTTGAGTCCTCCGA | |
| | GAAGCCATTCCTCTGGGTGATCAGAGCCCCGTTCGGCTTCGACATCAA | |
| | CGAAGAGATGCGCCCAGAGTGGCTGCCAGAGGGCTTTGAGGAACGCAT | |
| | GAAGGTGAAGAAACAGGGCAAGCTCGTGTACAAGCTCGGCCCGCAGCT | |
| | TGAGATCCTCAACCATGAATCCATCGGCGGCTTTCTCACCCACTGCGG | |
| | ATGGAACAGCATCCTTGAGTCTCTTCGCGAGGGCGTTCCGATGCTTGG | |
| | ATGGCCACTTGCTGCCGAGCAGGCCTACAACCTCAAGTACCTCGAAGA | |
| | TGAGATGGGCGTCGCGGTTGAGCTTGCTAGAGGCCTCGAAGGCGAGAT | |
| | CTCCAAAGAGAAGGTCAAGCGCATCGTCGAGATGATCCTTGAGCGCAA | |
| | CGAGGGCTCCAAAGGCTGGGAGATGAAGAATCGCGCCGTGGAAATGGG | |
| | CAAAAAGCTCAAGGACGCCGTGAACGAGGAAAAAGAGCTGAAGGGCTC | |
| | CTCCGTGAAGGCGATCGACGATTTCCTCGACGCCGTCATGCAGGCCAA | |
| | ACTTGAGCCAAGCCTCCAGTGATAGTGAGCTTGAAGTGACATCACAAA | |
| | GTTGAAGGTAATAAAGCCAAATTAATTAAGACATTTTCATAATGATGT | |
| | CAAGAATGCAAAGCAAATTGCATAACTGCCTTTATGCAAAACATTAAT | |
| | ATAATATAAATTATAAAGAACTGCGCTCTCTGCTTCTTATTTTCTTAG | |
| | CTTCATTTATTAGTCACTAGCTGTTCAGAATTTTCAGTATCTTTTGAT | |
| | ATTACTAAGAACCTAATCACACAATGTATATTCTTATGCAGGAAAAGC | |
| | AGAATGCTGAGCTAAAAGAAAGGCTTTTTCCATTTTCGAGAGACAATG | |
| | AGAAAAGAAGAAGAAGAAGAAGAAGAAGAAGAAGAAGAAGAAGAAAAG | |
| | AGTAAATAATAAAGCCCCACAGGAGGCGAAGTTCTTGTAGCTCCATGT | |
| | TATCTAAGTTATTGATATTGTTTGCCCTATATTTTATTTCTGTCATTG | |
| | TGTATGTTTTGTTCAGTTTCGATCTCCTTGCAAAATGCAGAGATTATG | |
| | AGATGAATAAACTAAGTTATATTATTATACGTGTTAATATTCTCCTCC | |
| | TCTCTCTAGCTAGCCTTTTGTTTTCTCTTTTTCTTATTTGATTTTCTT | |
| | TAAATCAATCCATTTTAGGAGAGGGCCAGGGAGTGATCCAGCAAAACA | |
| | TGAAGATTAGAAGAAACTTCCCTCTTTTTTTCCTGAAAACAATTTAAC | |
| | GTCGAGATTTATCTCTTTTTGTAATGGAATCATTTCTACAGTTATGAC | |
| | GGTAATATGAAGATGAAGATGAAATATTTGGTGTGTCAAATAAAAAGC | |
| | TTGTGTGCTTAAGTTTGTGTTTTTTTCTTGGCTTGTTGTGTTATGAAT | |
| | TTGTGGCTTTTTCTAATATTAAATGAATGTAAGATCTCATTATAATGA | |
| | ATAAACAAATGTTTCTATAATCCATTGTGAATGTTTTGTTGGATCTCT | |
| | TCTGCAGCATATAACTACTGTATGTGCTATGGTATGGACTATGGAATA | |
| | TGATTAAAGATAAGCGCTTTACGAATTCCCATGGGGAGAGGAAGAAGT | |
| | TCCCAATCTTGAAATATCGAAGGTTCGCCTTTCTGTTGTAATATTGCT | |
| | CAATCCTCTACTTTTGTGCCGCCATCTGTATCAAAGTTGCTTCCCTTA | |
| | TCTCCTTTGTCAAATCTAAATTTGTTAAAAGTTCTTTCTCATTTGTTA | |
| | CTTCATTTGTATGTACGTATCTTGTTCTTGGCTCTCCTATTTCCACCG | |
| | AAATTAAGGCCTCAGTACCATAAATAAGTGAGAATGGTGTTTCTCTTG | |
| | TGCTTATCTTCGTCGTTGTCCTGTAATCCCATACTAGCCCAAGTAGTA | |
| | CTTCAGGGAACCTGCCTTTTGATGATTCTAACCGTTTCTTTATATTGT | |
| | TGACAATAATTGTATTTGTCGATTATGGTTTCCCATTAGCTATGGGAT | |
| | GGCAGTGCGCCGAAATAATTTGTTTAATTTCCCAGATTTTGAGAAAAT | |
| | CGGTAACTTTTGAACCTACGAATTGTGGCCCATTATCGCGTACTATTT | |
| | TTCTTGGGACACAAAATCGACATATTATGTTCCTCCAAATAATATCAA | |
| | CAACCTCTTTCTCTCGATCTATTTAAAGGCACATGCTTCTACCCACAT | |
| | AGAAAAATAATCAGTTAAGATTAATAAAAGTCGTTCCTTTCCTGGTGC | |
| | TTTAGGTAGAGGACCTACAATACATAACCCACTTCATAAACGGCCACG | |
| | GTGCTATGATTGAATGAAGAAACACTGCTGGTTGATGCATATTGTTTG | |
| | CACCCCGTTGACATTTATCACACCTTCTAATAAAAATTTTCGCTTTGT | |
| | GTTCTATTTTTTGCAAATAATACCGCCCCTTTTAGCGTCTTTAAAATG | |
| | ACCTTCCTCCAACGTGATTGCAACAATGTTCTTTATGTACTTCTTGCA | |
| | TCACATATTCAGTTTGTGATAGACCAAGGCACCGTGCTAAGGGTCCCA | |
| | TGAACATTTCGATACAAATTTTCTTCTATTAAGCAATATTGAGCAGCT | |
| | TTCACTCGCAATAATTGGGATTTTTAATCGACGCTCAGAGAATTCGCA | |
| | TGCGGAGCCAGAAGGTAATTATCCAAGATGTAGCATCAAGAATCCAAT | |
| | GTTTACGGGAAAAACTATGGAAGTATTATGTAAGCTCAGCAAGAAGCA | |
| | GATCAATATGCGGCACATATGCAACCTATGTTCAAAAATGAAGAATGT | |
| | ACAGATACAAGATCCTATACTGCCAGAATACGAAGAAGAATACGTAGA | |
| | AATTGAAAAAGAAGAACCAGGCGAAGAAAAGAATCTTGATGACGTAAG | |
| | CACTGACGACAACAATGAAAAGAAGAAGATAAGGTCGGTGATTGTGAA | |
| | AGAGACATAGAGGACACATGTAAGGTGGAAAATGTAAGGGCGGAAAGT | |
| | AACCTTATCACAAAGGAATCTTATCCCCCACTACTTATCCTTTTATAT | |
| | TTTTCCGTGTCATTTTTGCCCTTGAGTTTTCCTATATAAGGAACCAAG | |
| | TTCGGCATTTGTGAAAACAAGAAAAAATTTGGTGTAAGCTATTTTCTT | |
| | TGAAGTACTGAGGATACAACTTCAGAGAAATTTGTAAGTTTGTAATGA | |
| | TATCACTATCCTCTTTTCATCCATCAAGCACAACTGCTACCGCTACCG | |
| | CAGCTGCCGCAACCACACACCCTCCACAGCAATGCCCCGCATTCTCTT | |
| | CACCACCTTCGCATCTATCCTTGCCGCTACGACACCCTCGTCAACATC | |
| | TTGTGGTTAGGTGTGGAGGGGGTGGCTCTGCATCGGAGTCCGTATTCA | |
| | ACAGAGATTCAGCCGCAACTAGGGTTTCAAACGATCACTTGGATGTAT | |
| | CTAAAAGGGATGTAAAACTCAAAATTGCTATCATAGGGTTCGGAAACT | |
| | TTGGGCAATTTCTCGCAAAGACTATGGCAAAACAAGGTCATAGAGTAC | |
| | TAGCATATTCTCGATCAGACTATTCCCGGGCGGCAAAAGAGATTGGAG | |
| | TTGAATATTTTACTGATGCAGATGACTTGTGTGAGGAACATCCTGAAG | |
| | TAATTCTTTTGTGTACATCTATACTGTCTACTGAGAAGGTGTTGCGGT | |
| | CATTACCTCTCCATAGATTGCGCAGATCAACCCTGTTCGCTGATGTTT | |
| | TGAGTGTGAAAGAGTTCCCTCGCTCTTTATTTCTGCAACTCTTGCCCA | |
| | AAGATTTTGATATACTCTGTACCCACCCTATGTTTGGGCCAGACAGCG | |
| | GGAAAGACGGGTGGGGCGGACTCCCATTCGTATTTGACAAAGTAAGGG | |
| | TGGGCAGCGACCAATCTCGGACGTCAAGAGCTGAGGCATTTTTAGATG | |
| | TTTTTAGAAACGCCGGGTGTAGAATGGTGGAAATGTCATGCGTAGATC | |
| | ATGACAAACATGCCGCAGGCTCACAATTTATTACACACATGATGGGAA | |
| | GGGTACTGGAAAAACTAGCACTTGAAAATACGCCAATTAACACAAAGG | |
| | GATACGAATCATTACTAAATCTAGTTGATAACACAGCACGCGACAGCT | |
| | TTGAATTGTTTTACGGCCTATTCCTTTATAACAAGAATGCTATGGAGC | |
| | AGTTGGATAGAATGGACTGGGCTTTTGAGATGGTGAAAAAGCAGCTAA | |
| | GCGGTTATTTGCATGATCTGGTGAGGAAACAACTAATGCTGGAAGGGA | |
| | ACAATGATCAGGCGGAGGTCACCTTCGATAAGCCCCTGATGCTACCCT | |
| | CTCCCACAATTAACCCACCTCAAATAGTGCCCTCCGCTGACATGGCTG | |
| | AGAAAAAGCACGATCTTGTAGTTGTCAATGGGACTAGGATGGCTTCTA | |
| | TGATATCCTCTTCAGCTGTGACTACAGTCAGCCGTGCTTCTACGGTGC | |
| | AATCGGCCGCGGTGGCTCCATTCGGCGGCCTCAAATCCATGACTGGAT | |
| | TCCCAGTTAAGAAGGTCAACACTGACATTACTTCCATTACAAGCAATG | |
| | GTGGAAGAGTAAAGTGCATGCAGATGAATTACCAGAACGATGACTTAA | |
| | GAATCAAAGAAATTAAAGAGTTGTTACCACCTGTGGCTCTTTTGGAAA | |
| | AATTCCCAGCAACTGAGAATGCTGCAAACACAGTTGCTCATGCAAGAA | |
| | AGGCTATTCACAAAATCTTGAAGGGTAATGATGACAGGTTACTTGTTG | |
| | TGATCGGACCATGCTCAATACATGATCCTGTTGCTGCAAAGGAATACG | |
| | CTACTAGATTGCTTGCATTGAGGGAAGAGTTAAAGGATGAACTTGAGA | |
| | TTGTTATGAGAGTGTACTTCGAGAAACCAAGGACCACTGTTGGTTGGA | |
| | AGGGACTTATCAATGATCCTCACATGGACAACTCCTTCCAAATTAATG | |
| | ATGGTTTGAGAATCGCTAGGAAACTTTTGCTTGATATTAACGACTCAG | |
| | GTTTGCCAGCTGCAGGAGAATTTTTAGATATGATCACACCTCAGTACT | |
| | TAGCTGACCTTATGTCATGGGGTGCTATAGGAGCAAGAACAACCGAAA | |
| | GTCAAGTTCATAGGGAGCAGGCTTCCGGTTTGTCTTGTCCAGTGGGAT | |
| | TCAAAAATGGTACTGATGGAACAATTAAGGTTGCTATAGACGCAATTA | |
| | ACGCTGCAGGTGCTCCTCATTGTTTTCTTTCTGTTACAAAATGGGGAC | |
| | ACTCAGCAATCGTGAATACCAGTGGTAACGGAGATTGCCATATTATCT | |
| | TGAGAGGTGGAAAAGAACCAAATTATTCAGCTAAGCACGTTGCAGAAG | |
| | TGAAAGAGGGTTTGAACAAGGCTGGATTACCTGCACAAGTTATGATCG | |
| | ATTTCTCTCATGCTAACTCCTCTAAGCAATTCAAGAAACAGATGGATG | |
| | TTTGTGCTGACGTGTGCCAACAGATCGCTGGTGGAGAAAAGGCTATTA | |
| | TTGGTGTTATGGTGGAAAGTCACTTAGTTGAGGGAAATCAATCATTAG | |
| | AAAGTGGAGAGCCTCTTGCTTACGGAAAATCTATTACCGATGCATGCA | |
| | TCGGTTGGGAAGATACTGACGCTCTTTTGAGACAGTTGGCTAACGCAG | |
| | TTAAGGCAAGAAGGGGTTGATGAGCTTGTCAAGCAGATCGTTCAAACA | |
| | TTTGGCAATAAAGTTTCTTAAGATTGAATCCTGTTGCCGGTCTTGCGA | |
| | TGATTATCATATAATTTCTGTTGAATTACGTTAAGCATGTAATAATTA | |
| | ACATGTAATGCATGACGTTATTTATGAGATGGGTTTTTATGATTAGAG | |
| | TCCCGCAATTATACATTTAATACGCGATAGAAAACAAAATATAGCGCG | |
| | CAAACTAGGATAAATTATCGCGCGCGGTGTCATCTATGTTACTAGATC | |
| | GACGCTTGTGTGAGACCGAGGATGCACATGTGACCGAGGGACACAGGT | |
| | AACCAACTCGGTCCATTTGCACCCCTAATCATAATAGCTTTAATATTT | |
| | CAAGATATTATTAAGTTAACGTTGTCAATATCCTGGAAATTTTGCAAA | |
| | ATGAATCAAGCCTATATGGCTGTAATATGAATTTAAAAGCAGCTCGAT | |
| | GTGGTGGTAATATGTAATTTACTTGATTCTAAAAAAATATCCCAAGTA | |
| | TTAATAATTTCTGCTAGGAAGAAGGTTAGCTACGATTTACAGCAAAGC | |
| | CAGAATACAAAGAACCATAAAGTGATTGAAGCTCGAAATATACGAAGG | |
| | AACAAATATTTTTAAAAAAATACGCAATGACTTGGAACAAAAGAAAGT | |
| | GATATATTTTTTGTTCTTAAACAAGCATCCCCTCTAAAGAATGGCAGT | |
| | TTTCCTTTGCATGTAACTATTATGCTCCCTTCGTTACAAAAATTTTGG | |
| | ACTACTATTGGGAACTTCTTCTGAAAATAGT | |
| PL-418 | TTCGTGTCCCTCGGTCACATGTGCATCCTCGGTCTCAGCTCAGCGATC | 189 |
| construct | TGGATTTTAGTACTGGATTTTGGTTTTAGGAATTAGAAATTTTATTGA | |
| | TAGAAGTATTTTACAAATACAAATACATACTAAGGGTTTCTTATATGC | |
| | TCAACACATGAGCGAAACCCTATAGGAACCCTAATTCCCTTATCTGGG | |
| | AACTACTCACACATTATTATGGAGAAACTCGAGCTTGTCGATCGACTC | |
| | TAGCTAGAGAAGCTTACTTGTACAGCTCGTCCATGCCGAGAGTGATCC | |
| | CGGCGGCGGTCACGAACTCCAGCAGGACCATGTGATCGCGCTTCTCGT | |
| | TGGGGTCTTTGCTCAGGGCGGACTGGGTGCTCAGGTAGTGGTTGTCGG | |
| | GCAGCAGCACGGGGCCGTCGCCGATGGGGGTGTTCTGCTGGTAGTGGT | |
| | CGGCGAGCTGCACGCTGCCGTCCTCGATGTTGTGGCGGATCTTGAAGT | |
| | TCACCTTGATGCCGTTCTTCTGCTTGTCGGCCATGATATAGACGTTGT | |
| | GGCTGTTGTAGTTGTACTCCAGCTTGTGCCCCAGGATGTTGCCGTCCT | |
| | CCTTGAAGTCGATGCCCTTCAGCTCGATGCGGTTCACCAGGGTGTCGC | |
| | CCTCGAACTTCACCTCGGCGCGGGTCTTGTAGTTGCCGTCGTCCTTGA | |
| | AGAAGATGGTGCGCTCCTGGACGTAGCCTTCGGGCATGGCGGACTTGA | |
| | AGAAGTCGTGCTGCTTCATGTGGTCGGGGTAGCGGCTGAAGCACTGCA | |
| | CGCCGTAGGTCAGGGTGGTCACGAGGGTGGGCCAGGGCACGGGCAGCT | |
| | TGCCGGTGGTGCAGATGAACTTCAGGGTCAGCTTGCCGTAGGTGGCAT | |
| | CGCCCTCGCCCTCGCCGGACACGCTGAACTTGTGGCCGTTTACGTCGC | |
| | CGTCCAGCTCGACCAGGATGGGCACCACCCCGGTGAACAGCTCCTCGC | |
| | CCTTGCTCACCATTCTGCAAAAGAGAACCAGACAACAGGGTAAGTGCC | |
| | TAGCAGTAAACAAACAGAACTCATCACAAGCAAACAGCAACATCATAT | |
| | TCATACCAACAGGTCATGTGTGTTCATCACATCATTAGTACTAAGCAT | |
| | GCCATCATCCAAGTATATCAAAGTAAGGGCAAAGAGCATGCATGATCA | |
| | TCAGGTGCACAAAAGAATCATCAAATTGTAGCAGTACATATCTTCATC | |
| | TATCATGCATATCTATCCATAACAGGACGATGCATGTTGACCAGGTAA | |
| | AAGCTACAGGATCCTATAGGAACAGCAGCGTATATATCTTCACCAATC | |
| | TTAGCATCTTAATCATGTGGCACATGCAGTTTCAATTTAAGCACATGA | |
| | GCTAGTTGATTATGAGGTACCAGAGAATCATCAAACTGATGTAGCAGC | |
| | ATATATCCTCATCTATCATGCAATCTAATCTAATCTAACTAAACAGGA | |
| | AAGGTGTGCTATTCAGTTAAAAGCTACCGCATCATATACAAACGGCAG | |
| | CATAAGAAAAAGCATAATCATCTTAATCATGCAACAAACGCAGATTCA | |
| | TAATAAGCCCAAGAGCTAGCTTGTGATGATCTTATTCTACTCTGATCT | |
| | ACAGCAATCAGATAACGACCTAACCTTGCACATGGCAACAAAACAATC | |
| | GATCGGACGAATCAGTTGTTTGTTCCTAGCTAGCACCATCGAACCAGA | |
| | TAATAGATGCACGTACAGATCCCGAAAACGAACCCAAAAACAGGGCAG | |
| | ACCTAGCTGAACCTAGGCAGCGACCCAGCAGATCGTGAGAACGATCTC | |
| | ATCTACGAACAGCCTAGAAGCAACCCCACGATTCCCGGACAAACGACC | |
| | TAAAATCCCCACAAATCACATGAGCATGACAGGATAAACAGCGGAACC | |
| | GATCAGATCTACACGAAAACCCCACCTCCCAGCCACCCACGATCAGGA | |
| | AACACGCGGATCTAGCATGATTTCGTCAACGCCTCAGCCTAGTTCCTA | |
| | GCCACAGACCAAGCAGAACCACCAAACCACGCCGAGCGAGGAGATGGG | |
| | GCAAGAGGACGGGGGAGACGATCGCCGTACCTTGAAGCGGGGGAAGGA | |
| | TCGCCGAGGGTCGCGAGAAGAGCAATTTGGATTTGGAACCCGGGGGTT | |
| | GTGCGCTCCGAACGATGAGACGATGTGAGATTGTGGGAAGAGGCGCGG | |
| | AGGGCCCTGTATTTATGGGCTGCGACGGGGGGAGGAGAGGTGGGGAGG | |
| | GTTGGGGAAGGAATCCCCCACCCGTGCCGTGACGGTTCCGGGCCGTGT | |
| | GAGAGGAGCCCGCTCGTCTCCGCCACGCAATTTCCGCGATCGGAGCGG | |
| | AGCTTTCGAGAGGCGGCTGGATGGTTGGTGGCCGTTAGATTTGTAGAC | |
| | GCCGTTAACGCCTCGCCTCCACCGGGAAGAGTTTTGAGCAGCCGCTTA | |
| | TGACAATGGCTTAACGACGTTAGACGGAGCGTTAGTGGCAGGCCATGA | |
| | TAGAATAGACGTATTGCAATGGGATTATAATTAAATAAATAAGAATAT | |
| | AATAAGATATGGCAAGTCGGCACTCATGACATGGTCTGCGAAATGATA | |
| | GTGCTCACTTTCTTAGCCGAGAAAGTTGACGCGACTGATTTAGAAGTT | |
| | AAGATATTATTTCTCTCTTCTTTTCTTTCCTCGTCATATAAGGATGAA | |
| | ATAAACTTTAGAGATTGCCGGTAGTGATTTTGGATTTCGGCGATCAGG | |
| | CTTGGTTTGCCGGTTTCGGACGGTGTGCCTTAGGCCACCCGCAGTGTA | |
| | TCTTGTAATGTTCAACCGATAAGCAAGGGTGGGGCTCAAGCAAGTAGT | |
| | AAACAACTATGTCAAATGTCACCATGGTTATGGTCTTGTTTAGTTGGC | |
| | TTCCTCCCTCGAGGAATTCCACAAGCGATCTGGATTTTAGTACTGGAT | |
| | TTTGGTTTTAGGAATTAGAAATTTTATTGATAGAAGTATTTTACAAAT | |
| | ACAAATACATACTAAGGGTTTCTTATATGCTCAACACATGAGCGAAAC | |
| | CCTATAGGAACCCTAATTCCCTTATCTGGGAACTACTCACACATTATT | |
| | ATGGAGAAACTCGAGCTTGTCGATCGACTCTAGCTAGAGAAGCCTACC | |
| | TAGTCCCATTGACAACTACAAGATCGTGCTTTTTCTCAGCCATGTCAG | |
| | CGGAGGGCACTATTTGAGGTGGGTTAATTGTGGGAGAGGGTAGCATCA | |
| | GGGGCTTATCGAAGGTGACCTCCGCCTGATCATTGTTCCCTTCCAGCA | |
| | TTAGTTGTTTCCTCACCAGATCATGCAAATAACCGCTTAGCTGCTTTT | |
| | TCACCATCTCAAAAGCCCAGTCCATTCTATCCAACTGCTCCATAGCAT | |
| | TCTTGTTATAAAGGAATAGGCCGTAAAACAATTCAAAGCTGTCGCGTG | |
| | CTGTGTTATCAACTAGATTTAGTAATGATTCGTATCCCTTTGTGTTAA | |
| | TTGGCGTATTTTCAAGTGCTAGTTTTTCCAGTACCCTTCCCATCATGT | |
| | GTGTAATAAATTGTGAGCCTGCGGCATGTTTGTCATGATCTACGCATG | |
| | ACATTTCCACCATTCTACACCCGGCGTTTCTAAAAACATCTAAAAATG | |
| | CCTCAGCTCTTGACGTCCGAGATTGGTCGCTGCCCACCCTTACTTTGT | |
| | CAAATACGAATGGGAGTCCGCCCCACCCGTCTTTCCCGCTGTCTGGCC | |
| | CAAACATAGGGTGGGTACAGAGTATATCAAAATCTTTGGGCAAGAGTT | |
| | GCAGAAATAAAGAGCGAGGGAACTCTTTCACACTCAAAACATCAGCGA | |
| | ACAGGGTTGATCTGCGCAATCTATGGAGAGGTAATGACCGCAACACCT | |
| | TCTCAGTAGACAGTATAGATGTACACAAAAGAATTACTTCAGGATGTT | |
| | CCTCACACAAGTCATCTGCATCAGTAAAATATTCAACTCCAATCTCTT | |
| | TTGCCGCCCGGGAATAGTCTGATCGAGAATATGCTAGTACTCTATGAC | |
| | CTTGTTTTGCCATAGTCTTTGCGAGAAATTGCCCAAAGTTTCCGAACC | |
| | CTATGATAGCAATTTTGAGTTTTACATCCCTTTTAGATACATCCAAGT | |
| | GATCGTTTGAAACCCTAGTTGCGGCTGAATCTCTGTTGAATACGGACT | |
| | CCGATGCAGAGCCACCCCCTCCACACCTAACCACAAGATGTTGACGAG | |
| | GGTGTCGTAGCGGCAAGGATAGATGCGAAGGTGGTGAAGAGAATGCGG | |
| | GGCATTGCTGTGGAGGGTGTGTGGTTGCGGCAGCTGCGGTAGCGGTAG | |
| | CAGTTGTGCTTGATGGATGAAAAGAGGATAGTGATATCATTCTGCAGA | |
| | AGTAACACCAAACAACAGGGTGAGCATCGACAAAAGAAACAGTACCAA | |
| | GCAAATAAATAGCGTATGAAGGCAGGGCTAAAAAAATCCACATATAGC | |
| | TGCTGCATATGCCATCATCCAAGTATATCAAGATCAAAATAATTATAA | |
| | AACATACTTGTTTATTATAATAGATAGGTACTCAAGGTTAGAGCATAT | |
| | GAATAGATGCTGCATATGCCATCATGTATATGCATCAGTAAAACCCAC | |
| | ATCAACATGTATACCTATCCTAGATCGATATTTCCATCCATCTTAAAC | |
| | TCGTAACTATGAAGATGTATGACACACACATACAGTTCCAAAATTAAT | |
| | AAATACACCAGGTAGTTTGAAACAGTATTCTACTCCGATCTAGAACGA | |
| | ATGAACGACCGCCCAACCACACCACATCATCACAACCAAGCGAACAAA | |
| | AAGCATCTCTGTATATGCATCAGTAAAACCCGCATCAACATGTATACC | |
| | TATCCTAGATCGATATTTCCATCCATCATCTTCAATTCGTAACTATGA | |
| | ATATGTATGGCACACACATACAGATCCAAAATTAATAAATCCACCAGG | |
| | TAGTTTGAAACAGAATTCTACTCCGATCTAGAACGACCGCCCAACCAG | |
| | ACCACATCATCACAACCAAGACAAAAAAAAGCATGAAAAGATGACCCG | |
| | ACAAACAAGTGCACGGCATATATTGAAATAAAGGAAAAGGGCAAACCA | |
| | AACCCTATGCAACGAAACAAAAAAAATCATGAAATCGATCCCGTCTGC | |
| | GGAACGGCTAGAGCCATCCCAGGATTCCCCAAAGAGAAACACTGGCAA | |
| | GTTAGCAATCAGAACGTGTCTGACGTACAGGTCGCATCCGTGTACGAA | |
| | CGCTAGCAGCACGGATCTAACACAAACACGGATCTAACACAAACATGA | |
| | ACAGAAGTAGAACTACCGGGCCCTAACCATGGACCGGAACGCCGATCT | |
| | AGAGAAGGTAGAGAGGGGGGGGGGGGAGGACGAGCGGCGTACCTTGAA | |
| | GCGGAGGTGCCGACGGGTGGATTTGGGGGAGATCTGGTTGTGTGTGTG | |
| | TGCGCTCCGAACAACACGAGGTTGGGGAAAGAGGGTGTGGAGGGGGTG | |
| | TCTATTTATTACGGCGGGCGAGGAAGGGAAAGCGAAGGAGCGGTGGGA | |
| | AAGGAATCCCCCGTAGCTGCCGGTGCCGTGAGAGGAGGAGGAGGCCGC | |
| | CTGCCGTGCCGGCTCACGTCTGCCGCTCCGCCACGCAATTTCTGGATG | |
| | CCGACAGCGGAGCAAGTCCAACGGTGGAGCGGAACTCTCGAGAGGGGT | |
| | CCAGAGGCAGCGACAGAGATGCCGTGCCGTCTGCTTCGCTTGGCCCGA | |
| | CGCGACGCTGCTGGTTCGCTGGTTGGTGTCCGTTAGACTCGTCGACGG | |
| | CGTTTAACAGGCTGGCATTATCTACTCGAAACAAGAAAAATGTTTCCT | |
| | TAGTTTTTTTAATTTCTTAAAGGGTATTTGTTTAATTTTTAGTCACTT | |
| | TATTTTATTCTATTTTATATCTAAATTATTAAATAAAAAAACTAAAAT | |
| | AGAGTTTTAGTTTTCTTAATTTAGAGGCTAAAATAGAATAAAATAGAT | |
| | GTACTAAAAAAATTAGTCTATAAAAACCATTAACCCTAAACCCTAAAT | |
| | GGATGTACTAATAAAATGGATGAAGTATTATATAGGTGAAGCTATTTG | |
| | CAAAAAAAAAGGAGAACACATGCACACTAAAAAGATAAAACTGTAGAG | |
| | TCCTGTTGTCAAAATACTCAATTGTCCTTTAGACCATGTCTAACTGTT | |
| | CATTTATATGATTCTCTAAAACACTGATATTATTGTAGTACTATAGAT | |
| | TATATTATTCGTAGAGTAAAGTTTAAATATATGTATAAAGATAGATAA | |
| | ACTGCACTTCAAACAAGTGTGACAAAAAAAATATGTGGTAATTTTTTA | |
| | TAACTTAGACATGCAATGCTCATTATCTCTAGAGAGGGGCACGACCGG | |
| | GTCACGCTGCACTGCAGCTCCGCATGCGAATTCTCTGAGCGATCTGGA | |
| | TTTTAGTACTGGATTTTGGTTTTAGGAATTAGAAATTTTATTGATAGA | |
| | AGTATTTTACAAATACAAATACATACTAAGGGTTTCTTATATGCTCAA | |
| | CACATGAGCGAAACCCTATAGGAACCCTAATTCCCTTATCTGGGAACT | |
| | ACTCACACATTATTATGGAGAAACTCGAGCTTGTCGATCGACTCTAGC | |
| | TAGAGAAGCTCAAGCATCTTCTTTAGATTTCTTTCCCCTACAAGAAAC | |
| | TATAAACTCTACAAAATTATCCATTGCCTTAACAGAAGATCCTTTGTA | |
| | ATCTCCCTCAATTTTCCAAGCATCCTTCAATTTCTCCCCAATCTCAAC | |
| | AGCTTTCTTCTTCATTTCCCACCCACAACTCCCCTCCTTTCTATCCAA | |
| | CACCATTTCCACCACACTCTTCACCCACTCTTTCTTCACCTCCCCTTC | |
| | CAACCCCCTCGTCAACTCGACCGCCACCCCCATTTCCTCCACCATCAT | |
| | CTTTGAATTATACGCTTGCTCGGCCGCCAACGGCCACCCGAGTATTGG | |
| | CACTCCTTCTCTTAAACCTTCCAATACCGAATTCCAACCACAATGACT | |
| | CAAAAATCCACCAGTAGCCTCATGCCTCAAAATATCCAACTGAGGCAC | |
| | CCAATCTCTCACCAACATCCCCTGTTTTTTCTCCATCATTCTTTCCTC | |
| | AAACTTCTCAGGCAGCCATTCGGGTTTAAACTCCCCATTGAAATCGAA | |
| | CCCGAATGGCGGCCGAATCACCCACAAGAACGGGATCTTACTCTCCTC | |
| | CAAACCTTTCGCTAACTCCATCATCTGAATCGGAGTCACTGTATTCTG | |
| | TGACCCGAAACAAATGTACAAAACTGAGTCATTTTCAAATTGATTCAA | |
| | CCACCTCAAACAAGCCTCTTCCTTCTCATCATCATCTTTTTTCGAAGA | |
| | TGTCATAATCAAAGGACCAATTCCCCAAATAGGAGATTTCGTGAAATT | |
| | CCTAAAAACTTGAAACCCTAACGGTTCAATCTCCTCGATGGAATTACA | |
| | CAACCACCCAGAACAATTCAAGGATGATTTAATCTGAGGTTGGAAAAA | |
| | CCTCGACCATTCATCGGACCCATCAGCAAACCTTAAAAACCGATGAAG | |
| | CTGATTACGTCGGAGTTTCCGATCTTCCGGGAAACCAGCCAATGAAAA | |
| | CTCCTCATCATCGCCGACATTCCGGTGAGGTAGATTGCTCCAGATTGA | |
| | AACATACGCACCGACACCGTAGGCCCCACCAGTGTTGAAGCAGATTCC | |
| | GGTGGACCCAACTGATCGGGCCACGTTATCGGCCCAGCCCAAGAACAC | |
| | GTCGAAGATTATGCACACGGGTGGGTCGTGCAGGTGGTGGCGCGTGAG | |
| | GTAGTCACGTACATGAGGTTCGAGTGAAGTGGATGCATGGAAGAGTTT | |
| | GATGATGGAGGTTAATGAGAGTTTATCGGTATTTTCGGTGTTGGGTGG | |
| | GACACCGTGGTCGGTGCTAGTGAAGGGGAGTTCGATGATGTTAAGGTT | |
| | GATGTTGAAGTTGTCGGATTGACGGCGGAGATTAGCGGCGTTTAAAGG | |
| | GGTAGTGAGGAGAGAGAGATTGAAGGGTGTTAGAGAGAGAAGGCGGTG | |
| | AGCGAGGTGGAGGAATGGTCGGAGATGGCCTTGTGCTAAAAATGGGAG | |
| | GAGGATTATGTGTTGTTGTTCTTGTTCTTTTGTTGATGACTCCATTCT | |
| | GCAAAAGAGAACCAGACAACAGGGTAAGTGCCTAGCAGTAAACAAACA | |
| | GAACTCATCACAAGCAAACAGCAACATCATATTCATACCAACAGGTCA | |
| | TGTGTGTTCATCACATCATTAGTACTAAGCATGCCATCATCCAAGTAT | |
| | ATCAAAGTAAGGGCAAAGAGCATGCATGATCATCAGGTGCACAAAAGA | |
| | ATCATCAAATTGTAGCAGTACATATCTTCATCTATCATGCATATCTAT | |
| | CCATAACAGGACGATGCATGTTGACCAGGTAAAAGCTACAGGATCCTA | |
| | TAGGAACAGCAGCGTATATATCTTCACCAATCTTAGCATCTTAATCAT | |
| | GTGGCACATGCAGTTTCAATTTAAGCACATGAGCTAGTTGATTATGAG | |
| | GTACCAGAGAATCATCAAACTGATGTAGCAGCATATATCCTCATCTAT | |
| | CATGCAATCTAATCTAATCTAACTAAACAGGAAAGGTGTGCTATTCAG | |
| | TTAAAAGCTACCGCATCATATACAAACGGCAGCATAAGAAAAAGCATA | |
| | ATCATCTTAATCATGCAACAAACGCAGATTCATAATAAGCCCAAGAGC | |
| | TAGCTTGTGATGATCTTATTCTACTCTGATCTACAGCAATCAGATAAC | |
| | GACCTAACCTTGCACATGGCAACAAAACAATCGATCGGACGAATCAGT | |
| | TGTTTGTTCCTAGCTAGCACCATCGAACCAGATAATAGATGCACGTAC | |
| | AGATCCCGAAAACGAACCCAAAAACAGGGCAGACCTAGCTGAACCTAG | |
| | GCAGCGACCCAGCAGATCGTGAGAACGATCTCATCTACGAACAGCCTA | |
| | GAAGCAACCCCACGATTCCCGGACAAACGACCTAAAATCCCCACAAAT | |
| | CACATGAGCATGACAGGATAAACAGCGGAACCGATCAGATCTACACGA | |
| | AAACCCCACCTCCCAGCCACCCACGATCAGGAAACACGCGGATCTAGC | |
| | ATGATTTCGTCAACGCCTCAGCCTAGTTCCTAGCCACAGACCAAGCAG | |
| | AACCACCAAACCACGCCGAGCGAGGAGATGGGGCAAGAGGACGGGGGA | |
| | GACGATCGCCGTACCTTGAAGCGGGGGAAGGATCGCCGAGGGTCGCGA | |
| | GAAGAGCAATTTGGATTTGGAACCCGGGGGTTGTGCGCTCCGAACGAT | |
| | GAGACGATGTGAGATTGTGGGAAGAGGCGCGGAGGGCCCTGTATTTAT | |
| | GGGCTGCGACGGGGGGAGGAGAGGTGGGGAGGGTTGGGGAAGGAATCC | |
| | CCCACCCGTGCCGTGACGGTTCCGGGCCGTGTGAGAGGAGCCCGCTCG | |
| | TCTCCGCCACGCAATTTCCGCGATCGGAGCGGAGCTTTCGAGAGGCGG | |
| | CTGGATGGTTGGTGGCCGTTAGATTTGTAGACGCCGTTAACGCCTCGC | |
| | CTCCACCGGGAAGAGTTTTGAGCAGCCGCTTATGACAATGGCTTAACG | |
| | ACGTTAGACGGAGCGTTAGTGGCAGGCCATGATAGAATAGACGTATTG | |
| | CAATGGGATTATAATTAAATAAATAAGAATATAATAAGATATGGCAAG | |
| | TCGGCACTCATGACATGGTCTGCGAAATGATAGTGCTCACTTTCTTAG | |
| | CCGAGAAAGTTGACGCGACTGATTTAGAAGTTAAGATATTATTTCTCT | |
| | CTTCTTTTCTTTCCTCGTCATATAAGGATGAAATAAACTTTAGAGATT | |
| | GCCGGTAGTGATTTTGGATTTCGGCGATCAGGCTTGGTTTGCCGGTTT | |
| | CGGACGGTGTGCCTTAGGCCACCCGCAGTGTATCTTGTAATGTTCAAC | |
| | CGATAAGCAAGGGTGGGGCTCAAGCAAGTAGTAAACAACTATGTCAAA | |
| | TGTCACCATGGTTATGGTCTTGTTTAGTTGGCTTCCTCCCCATGGGAA | |
| | TTCGTAAAGCGGATAATTTATTTGAAAATTCATAAGAAAAGCAAACGT | |
| | TACATGAATTGATGAAACAATACAAAGACAGATAAAGCCACGCACATT | |
| | TAGGATATTGGCCGAGATTACTGAATATTGAGTAAGATCACGGAATTT | |
| | CTGACAGGAGCATGTCTACAATTCAGCCCAAATGGCAGTTGAAATACT | |
| | CAAACCGCCCCATATGCAGGAGCGGATCATTCATTGTTTGTTTGGTTG | |
| | CCTTTGCCAACATGGGAGTCCAAGCTTAGGTGGAAGTAAACTTGTAGG | |
| | AGCCATGACATAAGGTACCATGGTCCCAACTAGTATGAATAAGCTCAG | |
| | CCTTCCACTTTTCACCAGCCGCCCCAACCACGACATGCAATGGATAAA | |
| | AATGCTCCGGGAATGGATGTGCCAATTCCCAGTTTGGTGCTTTGGTTT | |
| | CATATGTATTCACTTCTTCAAATCTTCCTTTAGTGAGAGCTACGTCAA | |
| | GCCATGAGTCAAAGTCAGCAGCCCAAGGAGCAACACCACCATGATAAT | |
| | GAGGGGTTTCGTCCAAAGGGTGTGTTGCACTTCCGGAACCAATAATGA | |
| | GTACACCTTCGTCTCTTAGAGGCGCTAATGCTCGTCCCAAGTTGTAGT | |
| | GGTACGTTCCGTCCAACTTCGGCTGGACGGAGAGCTGACATACCGGTA | |
| | TGTCAGCCTCCGGATACATGTACATAAGAGGCACCCATGCACCATGAT | |
| | CAAGCCCACGTTTTTTGTCAATGTGCACTGTGTCGAACCCGGATTTTT | |
| | TCATAATTTCTTCTACTCTGCTAGCCAAGTCTGGTGATCCTGGAGCTG | |
| | GATACTTCAACTTGTACATGGCTTGAGGATAGTCATCGAAATCGTAGA | |
| | TAGTATCATTGATATGAACAGCATTAACAGCAGGATGATCAGTTTCCC | |
| | AGTGACCAGAGATCACAAGTATCGCCTTCGGCTTCATCGTAAAGATTT | |
| | TTTCTCTCCATGTTTCAAAGAATGGCCTTAAGGGATGTGTATCTTCTA | |
| | CGGTCAAAATCGGGTTTCCATGGGTTATGAAGAAAGTTTCTTTGATCA | |
| | TGCTATTATCTTCTCCACTCATTTTCATTCTGCAGAAGTAACACCAAA | |
| | CAACAGGGTGAGCATCGACAAAAGAAACAGTACCAAGCAAATAAATAG | |
| | CGTATGAAGGCAGGGCTAAAAAAATCCACATATAGCTGCTGCATATGC | |
| | CATCATCCAAGTATATCAAGATCAAAATAATTATAAAACATACTTGTT | |
| | TATTATAATAGATAGGTACTCAAGGTTAGAGCATATGAATAGATGCTG | |
| | CATATGCCATCATGTATATGCATCAGTAAAACCCACATCAACATGTAT | |
| | ACCTATCCTAGATCGATATTTCCATCCATCTTAAACTCGTAACTATGA | |
| | AGATGTATGACACACACATACAGTTCCAAAATTAATAAATACACCAGG | |
| | TAGTTTGAAACAGTATTCTACTCCGATCTAGAACGAATGAACGACCGC | |
| | CCAACCACACCACATCATCACAACCAAGCGAACAAAAAGCATCTCTGT | |
| | ATATGCATCAGTAAAACCCGCATCAACATGTATACCTATCCTAGATCG | |
| | ATATTTCCATCCATCATCTTCAATTCGTAACTATGAATATGTATGGCA | |
| | CACACATACAGATCCAAAATTAATAAATCCACCAGGTAGTTTGAAACA | |
| | GAATTCTACTCCGATCTAGAACGACCGCCCAACCAGACCACATCATCA | |
| | CAACCAAGACAAAAAAAAGCATGAAAAGATGACCCGACAAACAAGTGC | |
| | ACGGCATATATTGAAATAAAGGAAAAGGGCAAACCAAACCCTATGCAA | |
| | CGAAACAAAAAAAATCATGAAATCGATCCCGTCTGCGGAACGGCTAGA | |
| | GCCATCCCAGGATTCCCCAAAGAGAAACACTGGCAAGTTAGCAATCAG | |
| | AACGTGTCTGACGTACAGGTCGCATCCGTGTACGAACGCTAGCAGCAC | |
| | GGATCTAACACAAACACGGATCTAACACAAACATGAACAGAAGTAGAA | |
| | CTACCGGGCCCTAACCATGGACCGGAACGCCGATCTAGAGAAGGTAGA | |
| | GAGGGGGGGGGGGGAGGACGAGCGGCGTACCTTGAAGCGGAGGTGCCG | |
| | ACGGGTGGATTTGGGGGAGATCTGGTTGTGTGTGTGTGCGCTCCGAAC | |
| | AACACGAGGTTGGGGAAAGAGGGTGTGGAGGGGGTGTCTATTTATTAC | |
| | GGCGGGCGAGGAAGGGAAAGCGAAGGAGCGGTGGGAAAGGAATCCCCC | |
| | GTAGCTGCCGGTGCCGTGAGAGGAGGAGGAGGCCGCCTGCCGTGCCGG | |
| | CTCACGTCTGCCGCTCCGCCACGCAATTTCTGGATGCCGACAGCGGAG | |
| | CAAGTCCAACGGTGGAGCGGAACTCTCGAGAGGGGTCCAGAGGCAGCG | |
| | ACAGAGATGCCGTGCCGTCTGCTTCGCTTGGCCCGACGCGACGCTGCT | |
| | GGTTCGCTGGTTGGTGTCCGTTAGACTCGTCGACGGCGTTTAACAGGC | |
| | TGGCATTATCTACTCGAAACAAGAAAAATGTTTCCTTAGTTTTTTTAA | |
| | TTTCTTAAAGGGTATTTGTTTAATTTTTAGTCACTTTATTTTATTCTA | |
| | TTTTATATCTAAATTATTAAATAAAAAAACTAAAATAGAGTTTTAGTT | |
| | TTCTTAATTTAGAGGCTAAAATAGAATAAAATAGATGTACTAAAAAAA | |
| | TTAGTCTATAAAAACCATTAACCCTAAACCCTAAATGGATGTACTAAT | |
| | AAAATGGATGAAGTATTATATAGGTGAAGCTATTTGCAAAAAAAAAGG | |
| | AGAACACATGCACACTAAAAAGATAAAACTGTAGAGTCCTGTTGTCAA | |
| | AATACTCAATTGTCCTTTAGACCATGTCTAACTGTTCATTTATATGAT | |
| | TCTCTAAAACACTGATATTATTGTAGTACTATAGATTATATTATTCGT | |
| | AGAGTAAAGTTTAAATATATGTATAAAGATAGATAAACTGCACTTCAA | |
| | ACAAGTGTGACAAAAAAAATATGTGGTAATTTTTTATAACTTAGACAT | |
| | GCAATGCTCATTATCTCTAGAGAGGGGCACGACCGGGTCACGCTGCAC | |
| | TGCAGCTCCGAGCTCGAATTCTAGTAGCGATCTGGATTTTAGTACTGG | |
| | ATTTTGGTTTTAGGAATTAGAAATTTTATTGATAGAAGTATTTTACAA | |
| | ATACAAATACATACTAAGGGTTTCTTATATGCTCAACACATGAGCGAA | |
| | ACCCTATAGGAACCCTAATTCCCTTATCTGGGAACTACTCACACATTA | |
| | TTATGGAGAAACTCGAGCTTGTCGATCGACTCTAGCTAGAGAAGCTCA | |
| | ATACCTAAGAACGGGAATAATCTGAAGAGGTTTAGTCTTTTGTAAGGC | |
| | AATCCCAAATTTCTCATCCATGTCTAGATCTTCTGCTTTCATACCTTC | |
| | TTCAAGCTTCCAATTGAAGAATTGAAGAAGCGTAGCTAACATCAAAGT | |
| | TAACATTCTAATAGCCAAATTCATCCCGGGGCATATTCTCCTTCCAGC | |
| | TCCAAAAGGTAAGAGTCCAAAATCTCTTCCTTTCACATCAATTTCGGA | |
| | TCCTAAAAACCTCTCAGGTAAAAACACGTCAGGTTTCACCCACGCTTG | |
| | AGGGTCCCTTCCAATGGCCCATAAATTAACAAGTATTTGTGCATCTTT | |
| | CGGCACAACATAGCCATATAATTCAACATCACTATCAGCTTTACGAGG | |
| | CAAGAGAAATACAGTTGGTGGGTGTAGCCGCAATGTTTCTTTGATAAT | |
| | GGCTTGTAAGTAAGGTAGCTTAATAATGTCTGATTCTTGAATTTGTCT | |
| | ATCCTTGCCCAAGACTTGCTCAATTTCTTCTTGAGCTTTTTCCATCAT | |
| | CTTAGGATTTCGAATTAACTCTGCCATTACCCATTCAAAAGTACTTGA | |
| | TGTAGTGTCAGTCCCAGCATCAAAAATATCCACGAGCAGATGATTTAT | |
| | CTCGCCCATACTGAGCTCTTTTTGTTTGTGGAGGTCAAGAAGAATGTC | |
| | TAGCACATCATCGGTCGTCTTAGTCGAAGCAGAATCCTGGCCGTTGCC | |
| | AATCCTTTGACATATTATGCTTTGAAAAACTGCAATCAACTTGTCAAA | |
| | ATTAGACGCCAATCGACGTCGAATTCCTGATGGGTCTAAGCATCCTAA | |
| | GATTGGAAAATAATCAGCGTAGTTAGGTTTGCCAATATCTTCCATAAT | |
| | ATTCCATATAAGTTCTTTGAATTGTTGAGATGTATGGGATTTATGGTG | |
| | GGCTAATTCGACCGAAAAGAATAATTTTGATAACAAATTAAGGGAGGT | |
| | TGTAAATGCTGCCTTGCCAATATCAACAGCTTGTCCTTTTTGTGCACA | |
| | TTCTTGTACATATTGGAAAAGCTGTTGCACTTTGGCATGTCTTAGGGT | |
| | TTGGCAAGCATCAAGTCTTTGAGGAGAAAGTAAATGAACGGCGGTGAT | |
| | TTTTCTAAAGTTCCGCCACTTTGGAGAAACAGGCAACCACGACATAGT | |
| | CAGCTTATGGTGATCCCCAGCAGTGACAGAATTAGGAACAGTGCGGTT | |
| | AGAAAGTGGGTGGTCTTTTTTTAAGAACATTTCTTTGGCTACTTCAGC | |
| | CGAGGATACAACAATAGTTGTGACACTTCCTAGACGTAATGATATCAA | |
| | TGGACCATGAATCTTAGCAAGATTAGCAAATGACTGGTGAGGTTTCTC | |
| | ACCAACTTCAAGGATGTTTCCGATTAGTGGCAATGGTTTAGGACCGGG | |
| | GGGAAGAAGTTTGGTAGTTTGGCTAGTGAATAACATTTTAATGAAATG | |
| | AAAAACAACAAACCAAATAGCAAGTATCATTGCTAGTGTTGCATGATC | |
| | CATTCTGCAAAAGAGAACCAGACAACAGGGTAAGTGCCTAGCAGTAAA | |
| | CAAACAGAACTCATCACAAGCAAACAGCAACATCATATTCATACCAAC | |
| | AGGTCATGTGTGTTCATCACATCATTAGTACTAAGCATGCCATCATCC | |
| | AAGTATATCAAAGTAAGGGCAAAGAGCATGCATGATCATCAGGTGCAC | |
| | AAAAGAATCATCAAATTGTAGCAGTACATATCTTCATCTATCATGCAT | |
| | ATCTATCCATAACAGGACGATGCATGTTGACCAGGTAAAAGCTACAGG | |
| | ATCCTATAGGAACAGCAGCGTATATATCTTCACCAATCTTAGCATCTT | |
| | AATCATGTGGCACATGCAGTTTCAATTTAAGCACATGAGCTAGTTGAT | |
| | TATGAGGTACCAGAGAATCATCAAACTGATGTAGCAGCATATATCCTC | |
| | ATCTATCATGCAATCTAATCTAATCTAACTAAACAGGAAAGGTGTGCT | |
| | ATTCAGTTAAAAGCTACCGCATCATATACAAACGGCAGCATAAGAAAA | |
| | AGCATAATCATCTTAATCATGCAACAAACGCAGATTCATAATAAGCCC | |
| | AAGAGCTAGCTTGTGATGATCTTATTCTACTCTGATCTACAGCAATCA | |
| | GATAACGACCTAACCTTGCACATGGCAACAAAACAATCGATCGGACGA | |
| | ATCAGTTGTTTGTTCCTAGCTAGCACCATCGAACCAGATAATAGATGC | |
| | ACGTACAGATCCCGAAAACGAACCCAAAAACAGGGCAGACCTAGCTGA | |
| | ACCTAGGCAGCGACCCAGCAGATCGTGAGAACGATCTCATCTACGAAC | |
| | AGCCTAGAAGCAACCCCACGATTCCCGGACAAACGACCTAAAATCCCC | |
| | ACAAATCACATGAGCATGACAGGATAAACAGCGGAACCGATCAGATCT | |
| | ACACGAAAACCCCACCTCCCAGCCACCCACGATCAGGAAACACGCGGA | |
| | TCTAGCATGATTTCGTCAACGCCTCAGCCTAGTTCCTAGCCACAGACC | |
| | AAGCAGAACCACCAAACCACGCCGAGCGAGGAGATGGGGCAAGAGGAC | |
| | GGGGGAGACGATCGCCGTACCTTGAAGCGGGGGAAGGATCGCCGAGGG | |
| | TCGCGAGAAGAGCAATTTGGATTTGGAACCCGGGGGTTGTGCGCTCCG | |
| | AACGATGAGACGATGTGAGATTGTGGGAAGAGGCGCGGAGGGCCCTGT | |
| | ATTTATGGGCTGCGACGGGGGGAGGAGAGGTGGGGAGGGTTGGGGAAG | |
| | GAATCCCCCACCCGTGCCGTGACGGTTCCGGGCCGTGTGAGAGGAGCC | |
| | CGCTCGTCTCCGCCACGCAATTTCCGCGATCGGAGCGGAGCTTTCGAG | |
| | AGGCGGCTGGATGGTTGGTGGCCGTTAGATTTGTAGACGCCGTTAACG | |
| | CCTCGCCTCCACCGGGAAGAGTTTTGAGCAGCCGCTTATGACAATGGC | |
| | TTAACGACGTTAGACGGAGCGTTAGTGGCAGGCCATGATAGAATAGAC | |
| | GTATTGCAATGGGATTATAATTAAATAAATAAGAATATAATAAGATAT | |
| | GGCAAGTCGGCACTCATGACATGGTCTGCGAAATGATAGTGCTCACTT | |
| | TCTTAGCCGAGAAAGTTGACGCGACTGATTTAGAAGTTAAGATATTAT | |
| | TTCTCTCTTCTTTTCTTTCCTCGTCATATAAGGATGAAATAAACTTTA | |
| | GAGATTGCCGGTAGTGATTTTGGATTTCGGCGATCAGGCTTGGTTTGC | |
| | CGGTTTCGGACGGTGTGCCTTAGGCCACCCGCAGTGTATCTTGTAATG | |
| | TTCAACCGATAAGCAAGGGTGGGGCTCAAGCAAGTAGTAAACAACTAT | |
| | GTCAAATGTCACCATGGTTATGGTCTTGTTTAGTTGGCTTCCTCCAAG | |
| | CTTGAATTCTTGCGGAGTCGAGGTCATTCATATGCTTGAGAAGAGAGT | |
| | CGGGATAGTCCAAAATAAAACAAAGGTAAGATTACCTGGTCAAAAGTG | |
| | AAAACATCAGTTAAAAGGTGGTATAAAGTAAAATATCGGTAATAAAAG | |
| | GTGGCCCAAAGTGAAATTTACTCTTTTCTACTATTATAAAAATTGAGG | |
| | ATGTTTTTGTCGGTACTTTGATACGTCATTTTTGTATGAATTGGTTTT | |
| | TAAGTTTATTCGCTTTTGGAAATGCATATCTGTATTTGAGTCGGGTTT | |
| | TAAGTTCGTTTGCTTTTGTAAATACAGAGGGATTTGTATAAGAAATAT | |
| | CTTTAAAAAAACCCATATGCTAATTTGACATAATTTTTGAGAAAAATA | |
| | TATATTCAGGCGAATTCTCACAATGAACAATAATAAGATTAAAATAGC | |
| | TTTCCCCCGTTGCAGCGCATGGGTATTTTTTCTAGTAAAAATAAAAGA | |
| | TAAACTTAGACTCAAAACATTTACAAAAACAACCCCTAAAGTTCCTAA | |
| | AGCCCAAAGTGCTATCCACGATCCATAGCAAGCCCAGCCCAACCCAAC | |
| | CCAACCCAACCCACCCCAGTCCAGCCAACTGGACAATAGTCTCCACAC | |
| | CCCCCCACTATCACCGTGAGTTGTCCGCACGCACCGCACGTCTCGCAG | |
| | CCAAAAAAAAAAAAAGAAAGAAAAAAAAGAAAAAGAAAAAACAGCAGG | |
| | TGGGTCCGGGTCGTGGGGGCCGGAAACGCGAGGAGGATCGCGAGCCAG | |
| | CGACGAGGCCGGCCCTCCCTCCGCTTCCAAAGAAACGCCCCCCATCGC | |
| | CACTATATACATACCCCCCCCTCTCCTCCCATCCCCCCAACCCTACCA | |
| | CCACCACCACCACCACCTCCACCTCCTCCCCCCTCGCTGCCGGACGAC | |
| | GAGCTCCTCCCCCCTCCCCCTCCGCCGCCGCCGCGCCGGTAACCACCC | |
| | CGCCCCTCTCCTCTTTCTTTCTCCGTTTTTTTTTTCCGTCTCGCTCTC | |
| | GATCTTTGGCCTTGGTAGTTTGGGTGGGCGAGAGGCGGCTTCGTGCGC | |
| | GCCCAGATCGGTGCGCGGGAGGGGGGGGATCTCGCGGCTGGGGCTCTC | |
| | GCCGGCGTGGATCCGGCCCGGATCTCGCGGGGAATGGGGCTCTCGGAT | |
| | GTAGATCTGCGATCCGCCGTTGTTGGGGGAGATGATGGGGGGTTTAAA | |
| | ATTTCCGCCATGCTAAACAAGATCAGGAAGAGGGGAAAAGGGCACTAT | |
| | GGTTTATATTTTTATATATTTCTGCTGCTTCGTCAGGCTTAGATGTGC | |
| | TAGATCTTTCTTTCTTCTTTTTGTGGGTAGAATTTGAATCCCTCAGCA | |
| | TTGTTCATCGGTAGTTTTTCTTTTCATGATTTGTGACAAATGCAGCCT | |
| | CGTGCGGAGCTTTTTTGTAGGTAGACGATAAATGCTTTCACTACGTCA | |
| | ATCTATAAGATTTTTCAAGCCAGCCACAAGAACTTTGTGTAGCTCTAG | |
| | ATATCTGCTTCAGCAAAAACCCGTGACCGTGTTCGGGATTTTGAATTT | |
| | GACCGAGGACAGTTTCTTTGATGAATCGAGACGATTGGACCCAGCAGG | |
| | GGCTGTTACAGCTGCGATTGAGATGCTGAGGGTCGGCTCCGATGTAGT | |
| | CGACGTTGGACCCGCTGCCTCCCACCCAGACGCACGTCCAGTCTCTCC | |
| | AGCTGACGAGATACGTCGGATCGCCCCTCTTTTAGACGCCCTTAGCGA | |
| | CCAGATGCATAGAGTGTCAATTGATAGCTTTCAGCCCGAAACACAGCG | |
| | CTACGCCTTAAAACGCGGCGTTGGCTATCTTAACGATATACAGGGTTT | |
| | TCCGGACCCGGCTCTCTATCCGGACATAGCAGAAGCAGATTGTAGACT | |
| | AGTTGTCATGCATAGCGCTCAACGCGATGGAATAGCTACAAGAACAGG | |
| | TCATTTAAGACCAGAAGATGCTCTCGATGAAATAGTTAGATTCTTTGA | |
| | AGCAAGGGTCAGTGCTTTGAGGAGAAGCGGGGTCGCAGCCGATCGCTT | |
| | GATATTAGATCCCGGCATGGGGTTCTTTCTAAGTCCTGCACCCGAAAC | |
| | TTCTCTTCATGTTCTCAGCAACCTCCAAAAACTAAAGTCTGCCTTGGG | |
| | GCTTCCACTTCTAGTAAGTGTATCACGCAAAAGTTTCCTTGGGGCGAC | |
| | TGTTGGCCTTCCAGTGAAAGATCTCGGGCCAGCTTCACTGGCTGCAGA | |
| | GCTGCACGCGATAGGTAACGGCGCTGACTATGTCAGAACCCATGCTCC | |
| | GGGCGATCTGAGATCCGCAATTACAATCTCAGAAACCCTTGCAAAGTT | |
| | TAGAAGCCGTGATGCTAGAGATAGGGGCCTTGATCATGCATAGGCTTG | |
| | TCCTGCTTTAATGAGATATGCGAGAAGCCTATGATCGCATGATATTTG | |
| | CTTTCAATTCTGTTGTGCACGTTGTAAAAAACCTGAGCATGTGTAGCT | |
| | CAGATCCTTACCGCCGGTTTCGGTTCATTCTAATGAATATATCACCCG | |
| | TTACTATCGTATTTTTATGAATAATATTCTCCGTTCAATTTACTGATT | |
| | GTACCCTACTACTTATATGTACAATATTAAAATGAAAACAATATATTG | |
| | TGCTGAATAGGTTTATAGCGACATCTATGATAGAGCGCCACAATAACA | |
| | AACAATTGCGTTTTATTATTACAAATCCAATTTTAAAAAAAGCGGCAG | |
| | AACCGGTCAAACCTAAAAGACTGATTACATAAATCTTATTCAAATTTC | |
| | AAAAGTGCCCCAGGGGCTAGTATCTACGACACACCGAGCGGCGAACTA | |
| | ATAACGCTCACTGAAGGGAACTCCGGTTCCCCGCCGGCGCGCATGGGT | |
| | GAGATTCCTTGAAGTTGAGTATTGGCCGTCCGCTCTACCGAAAGTTAC | |
| | GGGCACCATTCAACCCGGTCCAGCACGGCGGCCGGGTAACCGACTTGC | |
| | TGCCCCGAGAATTATGCAGCATTTTTTTGGTGTATGTGGGCCCCAAAT | |
| | GAAGTGCAGGTCAAACCTTGACAGTGACGACAAATCGTTGGGGGGGTC | |
| | CAGGGCGAATTTTGCGACAACATGTCGAGGCTCAGCAGGACCGCTGGA | |
| | TCCGAATTCGGCATGAGACCCACAGAGTGTTCAACCCCAGTACATTAA | |
| | AAACGTCCGCAATGTGTTATTAAGTTGTCTAAGCGTCAATTTGTTTAC | |
| | ACCACAATATATCCTGCCACCAGCCAGCCAACAGCTCCCCGACCGGCA | |
| | GCTCGGCACAAAATCACCACTCGATACAGGCAGCCCATCAACCATGAC | |
| | TACTGTCCGGTCCAACAGATCCGAGCAGGGGCTGCTGAACCCCCAGCC | |
| | GGAACTGACCCCACAAGGCCCTAGCGTTTGCAATGCACCAGGTCATCA | |
| | TTGACCCAGGCGTGTTCCACCAGGCCGCTGCCTCGCAACTCTTCGCAG | |
| | GCTTCGCCGACCTGCTCGCGCCACTTCTTCACGCGGGTGGAATCCGAT | |
| | CCGCACATGAGGCGGAAGGTTTCCAGCTTGAGCGGGTACGGCTCCCGG | |
| | TGCGAGCTGAAATAGTCGAACATCCGTCGGGCCGTCGGCGACAGCTTG | |
| | CGGTACTTCTCCCATATGAATTTCGTGTAGTGGTCGCCAGCAAACAGC | |
| | ACGACGATTTCCTCGTCGATCAGGACCTGGCAACGGGACGTTTTCTTG | |
| | CCACGGTCCAGGACGCGGAAGCGGTGCAGCAGCGACACCGATTCCAGG | |
| | TGCCCAACGCGGTCGGACGTGAAGCCCATCGCCGTCGCCTGTAGGCGC | |
| | GACAGGCATTCCTCGGCCTTCGTGTAATACCGGCCATTGATCGACCAG | |
| | CCCAGGTCCTGGCAAAGCTCGTAGAACGTGAAGGTGATCGGCTCGCCG | |
| | ATAGGGGTGCGCTTCGCGTACTCCAACACCTGCTGCCACACCAGTTCG | |
| | TCATCGTCGGCCCGCAGCTCGACGCCGGTGTAGGTGATCTTCACGTCC | |
| | TTGTTGACGTGGAAAATGACCTTGTTTTGCAGCGCCTCGCGCGGGATT | |
| | TTCTTGTTGCGCGTGGTGAACAGGGCAGAGCGGGCCGTGTCGTTTGGC | |
| | ATCGCTCGCATCGTGTCCGGCCACGGCGCAATATCGAACAAGGAAAGC | |
| | TGCATTTCCTTGATCTGCTGCTTCGTGTGTTTCAGCAACGCGGCCTGC | |
| | TTGGCCTCGCTGACCTGTTTTGCCAGGTCCTCGCCGGCGGTTTTTCGC | |
| | TTCTTGGTCGTCATAGTTCCTCGCGTGTCGATGGTCATCGACTTCGCC | |
| | AAACCTGCCGCCTCCTGTTCAAGACGACGCGAACGCTCCACGGCGGCC | |
| | GATGGCGCGGGCAGGGCAGGGGGAGCCAGTTGCACGCTGTCGCGCTCG | |
| | ATCTTGGCCGTAGCTTGCTGGACCATCGAGCCGACGGACTGGAAGGTT | |
| | TCGCGGGGCGCACGCATGACGGTGCGGCTTGCGATGGTTTCGGCATCC | |
| | TCGGCGGAAAACCCCGCGTCGATCAGTTCTTGCCTGTATGCCTTCCGG | |
| | TCAAACGTCCGATTCATTCACCCTCCTTGCGGGATTGCCCCGACTCAC | |
| | GCCGGGGCAATGTGCCCTTATTCCTGATTTGACCCGCCTGGTGCCTTG | |
| | GTGTCCAGATAATCCACCTTATCGGCAATGAAGTCGGTCCCGTAGACC | |
| | GTCTGGCCGTCCTTCTCGTACTTGGTATTCCGAATCTTGCCCTGCACG | |
| | AATACCAGCGACCCCTTGCCCAAATACTTGCCGTGGGCCTCGGCCTGA | |
| | GAGCCAAAACACTTGATGCGGAAGAAGTCGGTGCGCTCCTGCTTGTCG | |
| | CCGGCATCGTTGCGCCACATCTAGGATCTGCCAGGAACCGTAAAAAGG | |
| | CCGCGTTGCTGGCGTTTTTCCATAGGCTCCGCCCCCCTGACGAGCATC | |
| | ACAAAAATCGACGCTCAAGTCAGAGGTGGCGAAACCCGACAGGACTAT | |
| | AAAGATACCAGGCGTTTCCCCCTGGAAGCTCCCTCGTGCGCTCTCCTG | |
| | TTCCGACCCTGCCGCTTACCGGATACCTGTCCGCCTTTCTCCCTTCGG | |
| | GAAGCGTGGCGCTTTCTCATAGCTCACGCTGTAGGTATCTCAGTTCGG | |
| | TGTAGGTCGTTCGCTCCAAGCTGGGCTGTGTGCACGAACCCCCCGTTC | |
| | AGCCCGACCGCTGCGCCTTATCCGGTAACTATCGTCTTGAGTCCAACC | |
| | CGGTAAGACACGACTTATCGCCACTGGCAGCAGCCACTGGTAACAGGA | |
| | TTAGCAGAGCGAGGTATGTAGGCGGTGCTACAGAGTTCTTGAAGTGGT | |
| | GGCCTAACTACGGCTACACTAGAAGGACAGTATTTGGTATCTGCGCTC | |
| | TGCTGAAGCCAGTTACCTTCGGAAAAAGAGTTGGTAGCTCTTGATCCG | |
| | GCAAACAAACCACCGCTGGTAGCGGTGGTTTTTTTGTTTGCAAGCAGC | |
| | AGATTACGCGCAGAAAAAAAGGATCTCAAGAAGATCCTTTGATCTTTT | |
| | CTACGGGGTCTGACGCTCAGTGGAACGAAAACTCACGTTAAGGGATTT | |
| | TGGTCATGAGATTATCAAAAAGGATCTTCACCTAGATCCTTTTAAATT | |
| | AAAAATGAAGTTTTAAATCAATCTAAAGTATATATGAGTAAACTTGGT | |
| | CTGACAGCATGATATATCTCCCAATTTGTGTAGGGCTTATTATGCACG | |
| | CTTAAAAATAATAAAAGCAGACTTGACCTGATAGTTTGGCTGTGAGCA | |
| | ATTATGTGCTTAGTGCATCTAACGCTTGAGTTAAGCCGCGCCGCGAAG | |
| | CGGCGTCGGCTTGAACGAATTTCTAGCTAGACATTATTTGCCGACTAC | |
| | CTTGGTGATCTCGCCTTTCACGTAATGGACAAATTCTTCCAACTGATC | |
| | TGCGCGCGAGGCCAAGCGATCTTCTTCTTGTCCAAGATAAGCCTGTCT | |
| | AGCTTCAAGTATGACGGGCTGATACTGGGCCGGCAGGCGCTCCATTGC | |
| | CCAGTCGGCAGCGACATCCTTCGGCGCGATTTTGCCGGTTACTGCGCT | |
| | GTACCAAATGCGGGACAACGTAAGCACTACATTTCGCTCATCGCCAGC | |
| | CCAGTCGGGCGGCGAGTTCCATAGCGTTAAGGTTTCATTTAGCGCCTC | |
| | AAATAGATCCTGTTCAGGAACCGGATCAAAGAGTTCCTCCGCCGCTGG | |
| | ACCTACCAAGGCAACGCTATGTTCTCTTGCTTTTGTCAGCAAGATAGC | |
| | CAGATCAATGTCGATCGTGGCTGGCTCGAAGATACCTGCAAGAATGTC | |
| | ATTGCGCTGCCATTCTCCAAATTGCAGTTCGCGCTTAGCTGGATAACG | |
| | CCACGGAATGATGTCGTCGTGCACAACAATGGTGACTTCTACAGCGCG | |
| | GAGAATCTCGCTCTCTCCAGGGGAAGCCGAAGTTTCCAAAAGGTCGTT | |
| | GATCAAAGCTCGCCGCGTTGTTTCATCAAGCCTTACGGTCACCGTAAC | |
| | CAGCAAATCAATATCGCTGTGTGGCTTCAGGCCGCCATCCACTGCGGA | |
| | GCCGTACAAATGTACGGCCAGCAACGTCGGTTCGAGATGGCGCTCGAT | |
| | GACGCCAACTACCTCTGATAGTTGAGTCGATACTTCGGCGATCACCGC | |
| | TTCCCCCATGATGTTTAACTTTGTTTTAGGGCGACTGCCCTGCTGCGT | |
| | AACATCGTTGCTGCTCCATAACATCAAACATCGACCCACGGCGTAACG | |
| | CGCTTGCTGCTTGGATGCCCGAGGCATAGACTGTACCCCAAAAAAACA | |
| | GTCATAACAAGCCATGAAAACCGCCACTGCGCCGTTACCACCGCTGCG | |
| | TTCGGTCAAGGTTCTGGACCAGTTGCGTGAGCGCATACGCTACTTGCA | |
| | TTACAGCTTACGAACCGAACAGGCTTATGTCCACTGGGTTCGTGCCTC | |
| | GGGAATTGGCCAGCGCTGCCATTTTTGGGGTGAGGCCGTTCGCGGCCG | |
| | AGGGGCGCAGCCCCTGGGGGGATGGGAGGCCCGCGTTAGCGGGCCGGG | |
| | AGGGTTCGAGAAGGGGGGGCACCCCCCTTCGGCGTGCGCGGTCACGCG | |
| | CACAGGGCGCAGCCCTGGTTAAAAACAAGGTTTATAAATATTGGTTTA | |
| | AAAGCAGGTTAAAAGACAGGTTAGCGGTGGCCGAAAAACGGGCGGAAA | |
| | CCCTTGCAAATGCTGGATTTTCTGCCTGTGGACAGCCCCTCAAATGTC | |
| | AATAGGTGCGCCCCTCATCTGTCAGCACTCTGCCCCTCAAGTGTCAAG | |
| | GATCGCGCCCCTCATCTGTCAGTAGTCGCGCCCCTCAAGTGTCAATAC | |
| | CGCAGGGCACTTATCCCCAGGCTTGTCCACATCATCTGTGGGAAACTC | |
| | GCGTAAAATCAGGCGTTTTCGCCGATTTGCGAGGCTGGCCAGCTCCAC | |
| | GTCGCCGGCCGAAATCGAGCCTGCCCCTCATCTGTCAACGCCGCGCCG | |
| | GGTGAGTCGGCCCCTCAAGTGTCAACGTCCGCCCCTCATCTGTCAGTG | |
| | AGGGCCAAGTTTTCCGCGAGGTATCCACAACGCCGGCGGCCGCGGTGT | |
| | CTCGCACACGGCTTCGACGGCGTTTCTGGCGCGTTTGCAGGGCCATAG | |
| | ACGGCCGCCAGCCCAGCGGCGAGGGCAACCAGCCCGGTGAGCGTCGCA | |
| | AAGGCGGCTGGCACATACAAATGGACGAACGGATAAACCTTTTCACGC | |
| | CCTTTTAAATATCCGATTATTCTAATAAACGCTCTTTTCTCTTAGGTT | |
| | TACCCGCCAATATATCCTGTCAAACACTGATAGTTTAAACGGATCAC | |
| PL-450 | TGAACACTCTGTGGGTCTCATGCCGAATTCGGATCCGGAGGAAGCCAA | 190 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATCATGCAACACTAGCAATGATACTTGCTATT | |
| | TGGTTTGTTGTTTTTCATTTCATTAAAATGTTATTCACTAGCCAAACT | |
| | ACCAAACTTCTTCCCCCCGGTCCTAAACCATTGCCACTAATCGGAAAC | |
| | ATCCTTGAAGTTGGTGAGAAACCTCACCAGTCATTTGCTAATCTTGCT | |
| | AAGATTCATGGTCCATTGATATCATTACGTCTAGGAAGTGTCACAACT | |
| | ATTGTTGTATCCTCGGCTGAAGTAGCCAAAGAAATGTTCTTAAAAAAA | |
| | GACCACCCACTTTCTAACCGCACTGTTCCTAATTCTGTCACTGCTGGG | |
| | GATCACCATAAGCTGACTATGTCGTGGTTGCCTGTTTCTCCAAAGTGG | |
| | CGGAACTTTAGAAAAATCACCGCCGTTCATTTACTTTCTCCTCAAAGA | |
| | CTTGATGCTTGCCAAACCCTAAGACATGCCAAAGTGCAACAGCTTTTC | |
| | CAATATGTACAAGAATGTGCACAAAAAGGACAAGCTGTTGATATTGGC | |
| | AAGGCAGCATTTACAACCTCCCTTAATTTGTTATCAAAATTATTCTTT | |
| | TCGGTCGAATTAGCCCACCATAAATCCCATACATCTCAACAATTCAAA | |
| | GAACTTATATGGAATATTATGGAAGATATTGGCAAACCTAACTACGCT | |
| | GATTATTTTCCAATCTTAGGATGCTTAGACCCATCAGGAATTCGACGT | |
| | CGATTGGCGTCTAATTTTGACAAGTTGATTGCAGTTTTTCAAAGCATA | |
| | ATATGTCAAAGGATTGGCAACGGCCAGGATTCTGCTTCGACTAAGACG | |
| | ACCGATGATGTGCTAGACATTCTTCTTGACCTCCACAAACAAAAAGAG | |
| | CTCAGTATGGGCGAGATAAATCATCTGCTCGTGGATATTTTTGATGCT | |
| | GGGACTGACACTACATCAAGTACTTTTGAATGGGTAATGGCAGAGTTA | |
| | ATTCGAAATCCTAAGATGATGGAAAAAGCTCAAGAAGAAATTGAGCAA | |
| | GTCTTGGGCAAGGATAGACAAATTCAAGAATCAGACATTATTAAGCTA | |
| | CCTTACTTACAAGCCATTATCAAAGAAACATTGCGGCTACACCCACCA | |
| | ACTGTATTTCTCTTGCCTCGTAAAGCTGATAGTGATGTTGAATTATAT | |
| | GGCTATGTTGTGCCGAAAGATGCACAAATACTTGTTAATTTATGGGCC | |
| | ATTGGAAGGGACCCTCAAGCGTGGGTGAAACCTGACGTGTTTTTACCT | |
| | GAGAGGTTTTTAGGATCCGAAATTGATGTGAAAGGAAGAGATTTTGGA | |
| | CTCTTACCTTTTGGAGCTGGAAGGAGAATATGCCCCGGGATGAATTTG | |
| | GCTATTAGAATGTTAACTTTGATGTTAGCTACGCTTCTTCAATTCTTC | |
| | AATTGGAAGCTTGAAGAAGGTATGAAAGCAGAAGATCTAGACATGGAT | |
| | GAGAAATTTGGGATTGCCTTACAAAAGACTAAACCTCTTCAGATTATT | |
| | CCCGTTCTTAGGTATTGAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGACTGCAATCAAA | |
| | ATGAATACGAATGGTGAGGGTGAAACCCAACATATACTTATGATACCA | |
| | TTTATGGCCCAAGGGCACCTTCGACCGTTTCTGGAATTGGCCATGTTC | |
| | TTATACAAACGATCACATGTCATCATAACTCTGCTAACAACTCCCCTT | |
| | AACGCTGGGTTCCTGAGACATCTGTTGCATCACCATTCCTACAGCTCC | |
| | TCTGGGATACGGATCGTAGAATTGCCGTTTAATTCGACTAATCACGGG | |
| | CTCCCACCCGGTATAGAAAATACAGACAAATTAACACTGCCTTTAGTC | |
| | GTATCCTTGTTTCATTCTACAATTTCCCTAGACCCCCACCTACGCGAT | |
| | TATATTTCGAGACACTTCAGTCCTGCACGACCCCCACTGTGTGTTATT | |
| | CACGATGTTTTTCTCGGTTGGGTGGACCAAGTAGCTAAGGATGTAGGG | |
| | TCCACAGGCGTCGTTTTTACGACCGGCGGGGCTTATGGCACCTCTGCA | |
| | TATGTATCTATATGGAATGACTTACCACACCAAAATTACTCCGACGAT | |
| | CAGGAGTTTCCATTGCCAGGATTCCCCGAGAATCATAAATTTCGAAGA | |
| | TCTCAACTGCATCGTTTCCTTAGGTATGCTGATGGGTCTGATGACTGG | |
| | TCTAAATATTTCCAACCCCAACTACGGCAAAGTATGAAAAGTTTTGGG | |
| | TGGCTATGCAATAGTGTGGAAGAGATTGAAACATTGGGCTTCTCGATT | |
| | CTAAGAAACTACACCAAGCTTCCAATCTGGGGCATAGGCCCACTTATA | |
| | GCCTCCCCGGTACAGCACAGTTCAAGTGACAATAACTCTACTGGAGCA | |
| | GAGTTTGTGCAGTGGTTGAGTCTTAAAGAACCCGACAGTGTATTATAT | |
| | ATATCTTTCGGGAGTCAAAATACTATTTCACCAACTCAGATGATGGAA | |
| | TTGGCTGCAGGGTTGGAGTCGAGTGAGAAACCGTTTCTGTGGGTTATA | |
| | CGTGCCCCATTTGGGTTTGATATTAATGAGGAAATGCGTCCCGAATGG | |
| | CTTCCCGAGGGGTTTGAAGAGAGGATGAAAGTCAAAAAGCAAGGGAAG | |
| | CTTGTTTACAAATTGGGGCCTCAATTGGAAATCCTTAATCACGAAAGT | |
| | ATCGGGGGTTTTCTAACGCATTGCGGGTGGAACTCTATTCTTGAGTCC | |
| | CTAAGAGAAGGTGTTCCCATGTTGGGATGGCCACTAGCAGCTGAACAA | |
| | GCTTACAACCTAAAATATCTAGAGGATGAAATGGGTGTAGCAGTCGAA | |
| | CTAGCCAGAGGCCTCGAGGGGGAAATTTCCAAAGAAAAGGTTAAGAGA | |
| | ATAGTTGAGATGATCTTAGAGCGGAATGAAGGTTCAAAAGGATGGGAG | |
| | ATGAAAAACAGGGCAGTGGAAATGGGGAAAAAGTTAAAAGATGCCGTC | |
| | AACGAGGAAAAAGAGTTGAAAGGTTCTTCAGTCAAAGCAATAGACGAT | |
| | TTCCTTGACGCTGTGATGCAAGCAAAGCTCGAACCGTCATTGCAATGA | |
| | GCTTCTCTAGCTAGAGTCGATCGACAAGCTCGAGTTTCTCCATAATAA | |
| | TGTGTGAGTAGTTCCCAGATAAGGGAATTAGGGTTCCTATAGGGTTTC | |
| | GCTCATGTGTTGAGCATATAAGAAACCCTTAGTATGTATTTGTATTTG | |
| | TAAAATACTTCTATCAATAAAATTTCTAATTCCTAAAACCAAAATCCA | |
| | GTACTAAAATCCAGATCGCTCAGATGAGACCGAGGATGCACATGTGAC | |
| | CGAGGGACACGAA | |
| PL-451 | TGAACACTCTGTGGGTCTCATGCCGAATTCGGATCCGGAGGAAGCCAA | 191 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATCATGCAACACTAGCAATGATACTTGCTATT | |
| | TGGTTTGTTGTTTTTCATTTCATTAAAATGTTATTCACTAGCCAAACT | |
| | ACCAAACTTCTTCCCCCCGGTCCTAAACCATTGCCACTAATCGGAAAC | |
| | ATCCTTGAAGTTGGTGAGAAACCTCACCAGTCATTTGCTAATCTTGCT | |
| | AAGATTCATGGTCCATTGATATCATTACGTCTAGGAAGTGTCACAACT | |
| | ATTGTTGTATCCTCGGCTGAAGTAGCCAAAGAAATGTTCTTAAAAAAA | |
| | GACCACCCACTTTCTAACCGCACTGTTCCTAATTCTGTCACTGCTGGG | |
| | GATCACCATAAGCTGACTATGTCGTGGTTGCCTGTTTCTCCAAAGTGG | |
| | CGGAACTTTAGAAAAATCACCGCCGTTCATTTACTTTCTCCTCAAAGA | |
| | CTTGATGCTTGCCAAACCCTAAGACATGCCAAAGTGCAACAGCTTTTC | |
| | CAATATGTACAAGAATGTGCACAAAAAGGACAAGCTGTTGATATTGGC | |
| | AAGGCAGCATTTACAACCTCCCTTAATTTGTTATCAAAATTATTCTTT | |
| | TCGGTCGAATTAGCCCACCATAAATCCCATACATCTCAACAATTCAAA | |
| | GAACTTATATGGAATATTATGGAAGATATTGGCAAACCTAACTACGCT | |
| | GATTATTTTCCAATCTTAGGATGCTTAGACCCATCAGGAATTCGACGT | |
| | CGATTGGCGTCTAATTTTGACAAGTTGATTGCAGTTTTTCAAAGCATA | |
| | ATATGTCAAAGGATTGGCAACGGCCAGGATTCTGCTTCGACTAAGACG | |
| | ACCGATGATGTGCTAGACATTCTTCTTGACCTCCACAAACAAAAAGAG | |
| | CTCAGTATGGGCGAGATAAATCATCTGCTCGTGGATATTTTTGATGCT | |
| | GGGACTGACACTACATCAAGTACTTTTGAATGGGTAATGGCAGAGTTA | |
| | ATTCGAAATCCTAAGATGATGGAAAAAGCTCAAGAAGAAATTGAGCAA | |
| | GTCTTGGGCAAGGATAGACAAATTCAAGAATCAGACATTATTAAGCTA | |
| | CCTTACTTACAAGCCATTATCAAAGAAACATTGCGGCTACACCCACCA | |
| | ACTGTATTTCTCTTGCCTCGTAAAGCTGATAGTGATGTTGAATTATAT | |
| | GGCTATGTTGTGCCGAAAGATGCACAAATACTTGTTAATTTATGGGCC | |
| | ATTGGAAGGGACCCTCAAGCGTGGGTGAAACCTGACGTGTTTTTACCT | |
| | GAGAGGTTTTTAGGATCCGAAATTGATGTGAAAGGAAGAGATTTTGGA | |
| | CTCTTACCTTTTGGAGCTGGAAGGAGAATATGCCCCGGGATGAATTTG | |
| | GCTATTAGAATGTTAACTTTGATGTTAGCTACGCTTCTTCAATTCTTC | |
| | AATTGGAAGCTTGAAGAAGGTATGAAAGCAGAAGATCTAGACATGGAT | |
| | GAGAAATTTGGGATTGCCTTACAAAAGACTAAACCTCTTCAGATTATT | |
| | CCCGTTCTTAGGTATTGAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGGAGTCATCAACA | |
| | AAAGAACAAGAACAACAACACATAATCCTCCTCCCATTTTTAGCACAA | |
| | GGCCATCTCCGACCATTCCTCCACCTCGCTCACCGCCTTCTCTCTCTA | |
| | ACACCCTTCAATCTCTCTCTCCTCACTACCCCTTTAAACGCCGCTAAT | |
| | CTCCGCCGTCAATCCGACAACTTCAACATCAACCTTAACATCATCGAA | |
| | CTCCCCTTCACTAGCACCGACCACGGTGTCCCACCCAACACCGAAAAT | |
| | ACCGATAAACTCTCATTAACCTCCATCATCAAACTCTTCCATGCATCC | |
| | ACTTCACTCGAACCTCATGTACGTGACTACCTCACGCGCCACCACCTG | |
| | CACGACCCACCCGTGTGCATAATCTTCGACGTGTTCTTGGGCTGGGCC | |
| | GATAACGTGGCCCGATCAGTTGGGTCCACCGGAATCTGCTTCAACACT | |
| | GGTGGGGCCTACGGTGTCGGTGCGTATGTTTCAATCTGGAGCAATCTA | |
| | CCTCACCGGAATGTCGGCGATGATGAGGAGTTTTCATTGGCTGGTTTC | |
| | CCGGAAGATCGGAAACTCCGACGTAATCAGCTTCATCGGTTTTTAAGG | |
| | TTTGCTGATGGGTCCGATGAATGGTCGAGGTTTTTCCAACCTCAGATT | |
| | AAATCATCCTTGAATTGTTCTGGGTGGTTGTGTAATTCCATCGAGGAG | |
| | ATTGAACCGTTAGGGTTTCAAGTTTTTAGGAATTTCACGAAATCTCCT | |
| | ATTTGGGGAATTGGTCCTTTGATTATGACATCTTCGAAAAAAGATGAT | |
| | GATGAGAAGGAAGAGGCTTGTTTGAGGTGGTTGAATCAATTTGAAAAT | |
| | GACTCAGTTTTGTACATTTGTTTCGGGTCACAGAATACAGTGACTCCG | |
| | ATTCAGATGATGGAGTTAGCGAAAGGTTTGGAGGAGAGTAAGATCCCG | |
| | TTCTTGTGGGTGATTCGGCCGCCATTCGGGTTCGATTTCAATGGGGAG | |
| | TTTAAACCCGAATGGCTGCCTGAGAAGTTTGAGGAAAGAATGATGGAG | |
| | AAAAAACAGGGGATGTTGGTGAGAGATTGGGTGCCTCAGTTGGATATT | |
| | TTGAGGCATGAGGCTACTGGTGGATTTTTGAGTCATTGTGGTTGGAAT | |
| | TCGGTATTGGAAGGTTTAAGAGAAGGAGTGCCAATACTCGGGTGGCCG | |
| | TTGGCGGCCGAGCAAGCGTATAATTCAAAGATGATGGTGGAGGAAATG | |
| | GGGGTGGCGGTCGAGTTGACGAGGGGGTTGGAAGGGGAGGTGAAGAAA | |
| | GAGTGGGTGAAGAGTGTGGTGGAAATGGTGTTGGATAGAAAGGAGGGG | |
| | AGTTGTGGGTGGGAAATGAAGAAGAAAGCTGTTGAGATTGGGGAGAAA | |
| | TTGAAGGATGCTTGGAAAATTGAGGGAGATTACAAAGGATCTTCTGTT | |
| | AAGGCAATGGATAATTTTGTAGAGTTTATAGTTTCTTGTAGGGGAAAG | |
| | AAATCTAAAGAAGATGCTTGAGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTCAGAGAA | |
| | TTCGCATGCGGAGGAAGCCAACTAAACAAGACCATAACCATGGTGACA | |
| | TTTGACATAGTTGTTTACTACTTGCTTGAGCCCCACCCTTGCTTATCG | |
| | GTTGAACATTACAAGATACACTGCGGGTGGCCTAAGGCACACCGTCCG | |
| | AAACCGGCAAACCAAGCCTGATCGCCGAAATCCAAAATCACTACCGGC | |
| | AATCTCTAAAGTTTATTTCATCCTTATATGACGAGGAAAGAAAAGAAG | |
| | AGAGAAATAATATCTTAACTTCTAAATCAGTCGCGTCAACTTTCTCGG | |
| | CTAAGAAAGTGAGCACTATCATTTCGCAGACCATGTCATGAGTGCCGA | |
| | CTTGCCATATCTTATTATATTCTTATTTATTTAATTATAATCCCATTG | |
| | CAATACGTCTATTCTATCATGGCCTGCCACTAACGCTCCGTCTAACGT | |
| | CGTTAAGCCATTGTCATAAGCGGCTGCTCAAAACTCTTCCCGGTGGAG | |
| | GCGAGGCGTTAACGGCGTCTACAAATCTAACGGCCACCAACCATCCAG | |
| | CCGCCTCTCGAAAGCTCCGCTCCGATCGCGGAAATTGCGTGGCGGAGA | |
| | CGAGCGGGCTCCTCTCACACGGCCCGGAACCGTCACGGCACGGGTGGG | |
| | GGATTCCTTCCCCAACCCTCCCCACCTCTCCTCCCCCCGTCGCAGCCC | |
| | ATAAATACAGGGCCCTCCGCGCCTCTTCCCACAATCTCACATCGTCTC | |
| | ATCGTTCGGAGCGCACAACCCCCGGGTTCCAAATCCAAATTGCTCTTC | |
| | TCGCGACCCTCGGCGATCCTTCCCCCGCTTCAAGGTACGGCGATCGTC | |
| | TCCCCCGTCCTCTTGCCCCATCTCCTCGCTCGGCGTGGTTTGGTGGTT | |
| | CTGCTTGGTCTGTGGCTAGGAACTAGGCTGAGGCGTTGACGAAATCAT | |
| | GCTAGATCCGCGTGTTTCCTGATCGTGGGTGGCTGGGAGGTGGGGTTT | |
| | TCGTGTAGATCTGATCGGTTCCGCTGTTTATCCTGTCATGCTCATGTG | |
| | ATTTGTGGGGATTTTAGGTCGTTTGTCCGGGAATCGTGGGGTTGCTTC | |
| | TAGGCTGTTCGTAGATGAGATCGTTCTCACGATCTGCTGGGTCGCTGC | |
| | CTAGGTTCAGCTAGGTCTGCCCTGTTTTTGGGTTCGTTTTCGGGATCT | |
| | GTACGTGCATCTATTATCTGGTTCGATGGTGCTAGCTAGGAACAAACA | |
| | ACTGATTCGTCCGATCGATTGTTTTGTTGCCATGTGCAAGGTTAGGTC | |
| | GTTATCTGATTGCTGTAGATCAGAGTAGAATAAGATCATCACAAGCTA | |
| | GCTCTTGGGCTTATTATGAATCTGCGTTTGTTGCATGATTAAGATGAT | |
| | TATGCTTTTTCTTATGCTGCCGTTTGTATATGATGCGGTAGCTTTTAA | |
| | CTGAATAGCACACCTTTCCTGTTTAGTTAGATTAGATTAGATTGCATG | |
| | ATAGATGAGGATATATGCTGCTACATCAGTTTGATGATTCTCTGGTAC | |
| | CTCATAATCAACTAGCTCATGTGCTTAAATTGAAACTGCATGTGCCAC | |
| | ATGATTAAGATGCTAAGATTGGTGAAGATATATACGCTGCTGTTCCTA | |
| | TAGGATCCTGTAGCTTTTACCTGGTCAACATGCATCGTCCTGTTATGG | |
| | ATAGATATGCATGATAGATGAAGATATGTACTGCTACAATTTGATGAT | |
| | TCTTTTGTGCACCTGATGATCATGCATGCTCTTTGCCCTTACTTTGAT | |
| | ATACTTGGATGATGGCATGCTTAGTACTAATGATGTGATGAACACACA | |
| | TGACCTGTTGGTATGAATATGATGTTGCTGTTTGCTTGTGATGAGTTC | |
| | TGTTTGTTTACTGCTAGGCACTTACCCTGTTGTCTGGTTCTCTTTTGC | |
| | AGAATGATATCACTATCCTCTTTTCATCCATCAAGCACAACTGCTACC | |
| | GCTACCGCAGCTGCCGCAACCACACACCCTCCACAGCAATGCCCCGCA | |
| | TTCTCTTCACCACCTTCGCATCTATCCTTGCCGCTACGACACCCTCGT | |
| | CAACATCTTGTGGTTAGGTGTGGAGGGGGTGGCTCTGCATCGGAGTCC | |
| | GTATTCAACAGAGATTCAGCCGCAACTAGGGTTTCAAACGATCACTTG | |
| | GATGTATCTAAAAGGGATGTAAAACTCAAAATTGCTATCATAGGGTTC | |
| | GGAAACTTTGGGCAATTTCTCGCAAAGACTATGGCAAAACAAGGTCAT | |
| | AGAGTACTAGCATATTCTCGATCAGACTATTCCCGGGCGGCAAAAGAG | |
| | ATTGGAGTTGAATATTTTACTGATGCAGATGACTTGTGTGAGGAACAT | |
| | CCTGAAGTAATTCTTTTGTGTACATCTATACTGTCTACTGAGAAGGTG | |
| | TTGCGGTCATTACCTCTCCATAGATTGCGCAGATCAACCCTGTTCGCT | |
| | GATGTTTTGAGTGTGAAAGAGTTCCCTCGCTCTTTATTTCTGCAACTC | |
| | TTGCCCAAAGATTTTGATATACTCTGTACCCACCCTATGTTTGGGCCA | |
| | GACAGCGGGAAAGACGGGTGGGGCGGACTCCCATTCGTATTTGACAAA | |
| | GTAAGGGTGGGCAGCGACCAATCTCGGACGTCAAGAGCTGAGGCATTT | |
| | TTAGATGTTTTTAGAAACGCCGGGTGTAGAATGGTGGAAATGTCATGC | |
| | GTAGATCATGACAAACATGCCGCAGGCTCACAATTTATTACACACATG | |
| | ATGGGAAGGGTACTGGAAAAACTAGCACTTGAAAATACGCCAATTAAC | |
| | ACAAAGGGATACGAATCATTACTAAATCTAGTTGATAACACAGCACGC | |
| | GACAGCTTTGAATTGTTTTACGGCCTATTCCTTTATAACAAGAATGCT | |
| | ATGGAGCAGTTGGATAGAATGGACTGGGCTTTTGAGATGGTGAAAAAG | |
| | CAGCTAAGCGGTTATTTGCATGATCTGGTGAGGAAACAACTAATGCTG | |
| | GAAGGGAACAATGATCAGGCGGAGGTCACCTTCGATAAGCCCCTGATG | |
| | CTACCCTCTCCCACAATTAACCCACCTCAAATAGTGCCCTCCGCTGAC | |
| | ATGGCTGAGAAAAAGCACGATCTTGTAGTTGTCAATGGGACTAGGTAG | |
| | GCTTCTCTAGCTAGAGTCGATCGACAAGCTCGAGTTTCTCCATAATAA | |
| | TGTGTGAGTAGTTCCCAGATAAGGGAATTAGGGTTCCTATAGGGTTTC | |
| | GCTCATGTGTTGAGCATATAAGAAACCCTTAGTATGTATTTGTATTTG | |
| | TAAAATACTTCTATCAATAAAATTTCTAATTCCTAAAACCAAAATCCA | |
| | GTACTAAAATCCAGATCGCTTGTGTGAGACCGAGGATGCACATGTGAC | |
| | CGAGGGACACGAA | |
| PL-454 | TGAACACTCTGTGGGTCTCATGCCGAATTCGGATCCGGAGGAAGCCAA | 192 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGACCATGCGACCCTTGCAATGATACTAGCCATT | |
| | TGGTTCATCAGTTTTCACTTCATTAAACTACTCTTTAGCCAGCAAACC | |
| | ACTAAACTCCTACCACCGGGTCCCAAACCATTGCCAATCATAGGAAAC | |
| | ATCCTTGAGGTGGGAAAGAAACCACACCGGTCGTTCGCTAATCTTGCT | |
| | AAAATTCACGGGCCATTAATTAGTCTTAGACTCGGGTCAGTGACTACA | |
| | ATAGTTGTGAGCTCTGCCGATGTTGCCAAAGAAATGTTTCTAAAGAAA | |
| | GATCATCCTCTAAGTAATCGTACCATACCCAACAGCGTTACTGCGGGT | |
| | GATCATCACAAACTAACCATGTCGTGGTTACCAGTGTCTCCCAAGTGG | |
| | AGGAATTTTCGGAAAATAACCGCTGTGCACTTGCTCAGTCCCCAACGG | |
| | CTCGACGCATGTCAAACATTTAGACATGCCAAAGTCCAACAGCTATAC | |
| | GAATATGTTCAAGAATGCGCTCAGAAGGGTCAAGCAGTAGACATCGGT | |
| | AAGGCAGCTTTCACTACAAGTCTTAACCTACTTTCAAAATTATTTTTC | |
| | TCAGTCGAGTTGGCTCACCATAAAAGTCAGACATCACAAGAATTCAAG | |
| | GAGCTAATCTGGAACATAATGGAAGATATCGGTAAACCCAACTATGCA | |
| | GATTATTTTCCTATATTGGGCTGCGTCGACCCCAGTGGTATACGGAGA | |
| | CGATTGGCATGTTCATTTGACAAACTCATTGCTGTTTTTCAGTCTATA | |
| | ATTTGCGAAAGACTAGCTCCGGATAGTTCCACAGCAACTACAACCACA | |
| | ACTGACGATGTGCTTGATGTGTTGCTGCAGTTGTTCAAGCAAAATGAG | |
| | TTAACAATGGGTGAGATTAATCACCTTCTGGTGGATATATTCGACGCC | |
| | GGTACTGATACTACATCGTCTACTTTTGAGTGGGTGATGGCTGAGCTT | |
| | ATCAGGAACCCAGAAATGATGGAAAAAGCACAAGAAGAGATAAAACAA | |
| | GTGTTGGGGAAAGATAAACAAATCCAAGAGAGTGATATAATCAACTTG | |
| | CCTTATCTGCAAGCAATAATCAAGGAGACACTACGTCTACACCCTCCC | |
| | ACAGTCTTTCTCTTACCACGGAAAGCCGACACCGACGTGGAACTGTAC | |
| | GGATATATAGTTCCGAAAGATGCTCAAATTTTAGTCAACCTTTGGGCC | |
| | ATTGGGCGCGATCCCAATGCCTGGCAGAATGCCGATATTTTTAGTCCA | |
| | GAGAGATTTATCGGGTGTGAAATCGATGTTAAAGGTAGAGACTTCGGA | |
| | CTCTTACCCTTCGGGGCAGGCAGAAGGATATGTCCAGGAATGAACTTA | |
| | GCCATCCGAATGCTAACACTGATGCTAGCAACCCTTCTCCAATTCTTT | |
| | AATTGGAAGCTAGAAGGTGACATCTCGCCCAAAGACCTTGACATGGAC | |
| | GAGAAATTTGGAATTGCTCTTCAAAAAACAAAACCCTTAAAACTTATA | |
| | CCCATTCCCAGATATTAAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGACTGCAATCAAA | |
| | ATGAATACGAATGGTGAGGGTGAAACCCAACATATACTTATGATACCA | |
| | TTTATGGCCCAAGGGCACCTTCGACCGTTTCTGGAATTGGCCATGTTC | |
| | TTATACAAACGATCACATGTCATCATAACTCTGCTAACAACTCCCCTT | |
| | AACGCTGGGTTCCTGAGACATCTGTTGCATCACCATTCCTACAGCTCC | |
| | TCTGGGATACGGATCGTAGAATTGCCGTTTAATTCGACTAATCACGGG | |
| | CTCCCACCCGGTATAGAAAATACAGACAAATTAACACTGCCTTTAGTC | |
| | GTATCCTTGTTTCATTCTACAATTTCCCTAGACCCCCACCTACGCGAT | |
| | TATATTTCGAGACACTTCAGTCCTGCACGACCCCCACTGTGTGTTATT | |
| | CACGATGTTTTTCTCGGTTGGGTGGACCAAGTAGCTAAGGATGTAGGG | |
| | TCCACAGGCGTCGTTTTTACGACCGGCGGGGCTTATGGCACCTCTGCA | |
| | TATGTATCTATATGGAATGACTTACCACACCAAAATTACTCCGACGAT | |
| | CAGGAGTTTCCATTGCCAGGATTCCCCGAGAATCATAAATTTCGAAGA | |
| | TCTCAACTGCATCGTTTCCTTAGGTATGCTGATGGGTCTGATGACTGG | |
| | TCTAAATATTTCCAACCCCAACTACGGCAAAGTATGAAAAGTTTTGGG | |
| | TGGCTATGCAATAGTGTGGAAGAGATTGAAACATTGGGCTTCTCGATT | |
| | CTAAGAAACTACACCAAGCTTCCAATCTGGGGCATAGGCCCACTTATA | |
| | GCCTCCCCGGTACAGCACAGTTCAAGTGACAATAACTCTACTGGAGCA | |
| | GAGTTTGTGCAGTGGTTGAGTCTTAAAGAACCCGACAGTGTATTATAT | |
| | ATATCTTTCGGGAGTCAAAATACTATTTCACCAACTCAGATGATGGAA | |
| | TTGGCTGCAGGGTTGGAGTCGAGTGAGAAACCGTTTCTGTGGGTTATA | |
| | CGTGCCCCATTTGGGTTTGATATTAATGAGGAAATGCGTCCCGAATGG | |
| | CTTCCCGAGGGGTTTGAAGAGAGGATGAAAGTCAAAAAGCAAGGGAAG | |
| | CTTGTTTACAAATTGGGGCCTCAATTGGAAATCCTTAATCACGAAAGT | |
| | ATCGGGGGTTTTCTAACGCATTGCGGGTGGAACTCTATTCTTGAGTCC | |
| | CTAAGAGAAGGTGTTCCCATGTTGGGATGGCCACTAGCAGCTGAACAA | |
| | GCTTACAACCTAAAATATCTAGAGGATGAAATGGGTGTAGCAGTCGAA | |
| | CTAGCCAGAGGCCTCGAGGGGGAAATTTCCAAAGAAAAGGTTAAGAGA | |
| | ATAGTTGAGATGATCTTAGAGCGGAATGAAGGTTCAAAAGGATGGGAG | |
| | ATGAAAAACAGGGCAGTGGAAATGGGGAAAAAGTTAAAAGATGCCGTC | |
| | AACGAGGAAAAAGAGTTGAAAGGTTCTTCAGTCAAAGCAATAGACGAT | |
| | TTCCTTGACGCTGTGATGCAAGCAAAGCTCGAACCGTCATTGCAATGA | |
| | GCTTCTCTAGCTAGAGTCGATCGACAAGCTCGAGTTTCTCCATAATAA | |
| | TGTGTGAGTAGTTCCCAGATAAGGGAATTAGGGTTCCTATAGGGTTTC | |
| | GCTCATGTGTTGAGCATATAAGAAACCCTTAGTATGTATTTGTATTTG | |
| | TAAAATACTTCTATCAATAAAATTTCTAATTCCTAAAACCAAAATCCA | |
| | GTACTAAAATCCAGATCGCTCAGATGAGACCGAGGATGCACATGTGAC | |
| | CGAGGGACACGAA | |
| PL-455 | TGAACACTCTGTGGGTCTCATGCCGAATTCGGATCCGGAGGAAGCCAA | 193 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGACCATGCGACCCTTGCAATGATACTAGCCATT | |
| | TGGTTCATCAGTTTTCACTTCATTAAACTACTCTTTAGCCAGCAAACC | |
| | ACTAAACTCCTACCACCGGGTCCCAAACCATTGCCAATCATAGGAAAC | |
| | ATCCTTGAGGTGGGAAAGAAACCACACCGGTCGTTCGCTAATCTTGCT | |
| | AAAATTCACGGGCCATTAATTAGTCTTAGACTCGGGTCAGTGACTACA | |
| | ATAGTTGTGAGCTCTGCCGATGTTGCCAAAGAAATGTTTCTAAAGAAA | |
| | GATCATCCTCTAAGTAATCGTACCATACCCAACAGCGTTACTGCGGGT | |
| | GATCATCACAAACTAACCATGTCGTGGTTACCAGTGTCTCCCAAGTGG | |
| | AGGAATTTTCGGAAAATAACCGCTGTGCACTTGCTCAGTCCCCAACGG | |
| | CTCGACGCATGTCAAACATTTAGACATGCCAAAGTCCAACAGCTATAC | |
| | GAATATGTTCAAGAATGCGCTCAGAAGGGTCAAGCAGTAGACATCGGT | |
| | AAGGCAGCTTTCACTACAAGTCTTAACCTACTTTCAAAATTATTTTTC | |
| | TCAGTCGAGTTGGCTCACCATAAAAGTCAGACATCACAAGAATTCAAG | |
| | GAGCTAATCTGGAACATAATGGAAGATATCGGTAAACCCAACTATGCA | |
| | GATTATTTTCCTATATTGGGCTGCGTCGACCCCAGTGGTATACGGAGA | |
| | CGATTGGCATGTTCATTTGACAAACTCATTGCTGTTTTTCAGTCTATA | |
| | ATTTGCGAAAGACTAGCTCCGGATAGTTCCACAGCAACTACAACCACA | |
| | ACTGACGATGTGCTTGATGTGTTGCTGCAGTTGTTCAAGCAAAATGAG | |
| | TTAACAATGGGTGAGATTAATCACCTTCTGGTGGATATATTCGACGCC | |
| | GGTACTGATACTACATCGTCTACTTTTGAGTGGGTGATGGCTGAGCTT | |
| | ATCAGGAACCCAGAAATGATGGAAAAAGCACAAGAAGAGATAAAACAA | |
| | GTGTTGGGGAAAGATAAACAAATCCAAGAGAGTGATATAATCAACTTG | |
| | CCTTATCTGCAAGCAATAATCAAGGAGACACTACGTCTACACCCTCCC | |
| | ACAGTCTTTCTCTTACCACGGAAAGCCGACACCGACGTGGAACTGTAC | |
| | GGATATATAGTTCCGAAAGATGCTCAAATTTTAGTCAACCTTTGGGCC | |
| | ATTGGGCGCGATCCCAATGCCTGGCAGAATGCCGATATTTTTAGTCCA | |
| | GAGAGATTTATCGGGTGTGAAATCGATGTTAAAGGTAGAGACTTCGGA | |
| | CTCTTACCCTTCGGGGCAGGCAGAAGGATATGTCCAGGAATGAACTTA | |
| | GCCATCCGAATGCTAACACTGATGCTAGCAACCCTTCTCCAATTCTTT | |
| | AATTGGAAGCTAGAAGGTGACATCTCGCCCAAAGACCTTGACATGGAC | |
| | GAGAAATTTGGAATTGCTCTTCAAAAAACAAAACCCTTAAAACTTATA | |
| | CCCATTCCCAGATATTAAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGGATTCATCAAAA | |
| | ACAAAGCACATTATCCTCTTGCCATTTCTAGCGCAAGGTCATCTCCGT | |
| | CCATTTCTGCACTTAGCCAATTTCCTCAGATCCCACACTCCTTTCACC | |
| | CTTTCTATCCTTACTACTCCTCTCAATGCCGCCAATCTCCGCCGTCAA | |
| | TCCAACAACATTAACATCTACGATCTACCCTTTAACAGCACCGATCAT | |
| | AATCTACCACCAAACACAGAAAACACTGAAAAACTCCCATTAACTTCA | |
| | ATAATCTCTCTGTTTTATGCATCCACTTCGCTCCAGCCTCACGTACGC | |
| | AACCACCTTACTCGCCATCACCTTGATAACCCACCCATTTGTATCATC | |
| | TTTGATATTTTCTTGGGCTGGGCTGATAACTTGGCCCGATCTATTGGA | |
| | TCTACCGGAGTTTGTTTCAACACTGGTGGGGCCTACGGTTTGGCCGCT | |
| | TACATGTCGATTTGGATGCATTTGCCTCATAGAAATGTTCCAGACAAT | |
| | GTCGAGTTTTCCCTTCCGGAATTCCCTGAGAATCGGAAATTTAAACGT | |
| | AACCAACTTCACAGGTTTTTAAGATTTGCTGATGGAACCGATGATTGG | |
| | TCGGGTTTTTTTCAACCCCAGATAAAGTACTCCAGGAATTGTTCTGCG | |
| | TGGTTGTGTAATTCAATTGAGGAAATTGAGCCATTAGGGTTTGAGGTT | |
| | TTAAGAGCAATGTTGAAAGTACCAGTTTGGGGAATTGGTCCTTTAGTC | |
| | AAGACTGAAAATAGTTGTGATGAAGATGATGAACAAGGTTGTGTTGAG | |
| | TGGTTAAACCAATTTGAGAAAGGATCTGTTTTGTACATATCATTTGGG | |
| | TCACAGAATACAGTTACCCCGATCCAAATGATGGAATTAGCAAAAGGA | |
| | TTGGAGGAAAGTGGGGCGAAATTTTTGTGGGTGATTCGTCCCCCTTTC | |
| | GGGTTTGATATCAATGGAGAGTTCAAACCCGAATGGCTGCCGGAGGGA | |
| | TTTGAGAAGAGAGTAATGGAAAGGAAGCAGGGGAAGCTAGTAAAGAAA | |
| | TGGGGGCCTCAAATGGAGATATTGAGGAATAAGGCGACGGGGGCGTTT | |
| | TTGAGTCACTGTGGGTGGAATTCGTTGATCGAAGCGTTAAGCGAAGGT | |
| | GTTCCGATAATAGGGTGGCCATTGGCTGCGGAGCAAGCGTATAATTCA | |
| | AAGATGTTAGTGGAAGAGATGGGGGTTGCGGTCGAGTTAACGAGGGGA | |
| | TTGGAAGGGGAGTTGAGTAGCGAAGGGGTGAAGAAGGTGGTGGAGATG | |
| | GTTATGGATAGGGAAGAAGGGAGTTTTGGGTGTGAGATGAAGAAGGCG | |
| | GCAGTTGTAATTGGACAGAAATTGAGGGATGCAATGAAAGTGGAAGGT | |
| | GATTATAGAGGGTGTTCTCTTAGATCTCTTGATGAGTTTGTAGAGTTC | |
| | ATTGTTTCTCGTAAGGCTACTTTGAGCTAGGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTCAGATGAGACCGAGGATGCACATGTGACCGAGGGACACGAA | |
| PL-456 | TGAACACTCTGTGGGTCTCATGCCGAATTCGGATCCGGAGGAAGCCAA | 194 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGACCATGCGACCCTTGCAATGATACTAGCCATT | |
| | TGGTTCATCAGTTTTCACTTCATTAAACTACTCTTTAGCCAGCAAACC | |
| | ACTAAACTCCTACCACCGGGTCCCAAACCATTGCCAATCATAGGAAAC | |
| | ATCCTTGAGGTGGGAAAGAAACCACACCGGTCGTTCGCTAATCTTGCT | |
| | AAAATTCACGGGCCATTAATTAGTCTTAGACTCGGGTCAGTGACTACA | |
| | ATAGTTGTGAGCTCTGCCGATGTTGCCAAAGAAATGTTTCTAAAGAAA | |
| | GATCATCCTCTAAGTAATCGTACCATACCCAACAGCGTTACTGCGGGT | |
| | GATCATCACAAACTAACCATGTCGTGGTTACCAGTGTCTCCCAAGTGG | |
| | AGGAATTTTCGGAAAATAACCGCTGTGCACTTGCTCAGTCCCCAACGG | |
| | CTCGACGCATGTCAAACATTTAGACATGCCAAAGTCCAACAGCTATAC | |
| | GAATATGTTCAAGAATGCGCTCAGAAGGGTCAAGCAGTAGACATCGGT | |
| | AAGGCAGCTTTCACTACAAGTCTTAACCTACTTTCAAAATTATTTTTC | |
| | TCAGTCGAGTTGGCTCACCATAAAAGTCAGACATCACAAGAATTCAAG | |
| | GAGCTAATCTGGAACATAATGGAAGATATCGGTAAACCCAACTATGCA | |
| | GATTATTTTCCTATATTGGGCTGCGTCGACCCCAGTGGTATACGGAGA | |
| | CGATTGGCATGTTCATTTGACAAACTCATTGCTGTTTTTCAGTCTATA | |
| | ATTTGCGAAAGACTAGCTCCGGATAGTTCCACAGCAACTACAACCACA | |
| | ACTGACGATGTGCTTGATGTGTTGCTGCAGTTGTTCAAGCAAAATGAG | |
| | TTAACAATGGGTGAGATTAATCACCTTCTGGTGGATATATTCGACGCC | |
| | GGTACTGATACTACATCGTCTACTTTTGAGTGGGTGATGGCTGAGCTT | |
| | ATCAGGAACCCAGAAATGATGGAAAAAGCACAAGAAGAGATAAAACAA | |
| | GTGTTGGGGAAAGATAAACAAATCCAAGAGAGTGATATAATCAACTTG | |
| | CCTTATCTGCAAGCAATAATCAAGGAGACACTACGTCTACACCCTCCC | |
| | ACAGTCTTTCTCTTACCACGGAAAGCCGACACCGACGTGGAACTGTAC | |
| | GGATATATAGTTCCGAAAGATGCTCAAATTTTAGTCAACCTTTGGGCC | |
| | ATTGGGCGCGATCCCAATGCCTGGCAGAATGCCGATATTTTTAGTCCA | |
| | GAGAGATTTATCGGGTGTGAAATCGATGTTAAAGGTAGAGACTTCGGA | |
| | CTCTTACCCTTCGGGGCAGGCAGAAGGATATGTCCAGGAATGAACTTA | |
| | GCCATCCGAATGCTAACACTGATGCTAGCAACCCTTCTCCAATTCTTT | |
| | AATTGGAAGCTAGAAGGTGACATCTCGCCCAAAGACCTTGACATGGAC | |
| | GAGAAATTTGGAATTGCTCTTCAAAAAACAAAACCCTTAAAACTTATA | |
| | CCCATTCCCAGATATTAAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGGAGTCATCAACA | |
| | AAAGAACAAGAACAACAACACATAATCCTCCTCCCATTTTTAGCACAA | |
| | GGCCATCTCCGACCATTCCTCCACCTCGCTCACCGCCTTCTCTCTCTA | |
| | ACACCCTTCAATCTCTCTCTCCTCACTACCCCTTTAAACGCCGCTAAT | |
| | CTCCGCCGTCAATCCGACAACTTCAACATCAACCTTAACATCATCGAA | |
| | CTCCCCTTCACTAGCACCGACCACGGTGTCCCACCCAACACCGAAAAT | |
| | ACCGATAAACTCTCATTAACCTCCATCATCAAACTCTTCCATGCATCC | |
| | ACTTCACTCGAACCTCATGTACGTGACTACCTCACGCGCCACCACCTG | |
| | CACGACCCACCCGTGTGCATAATCTTCGACGTGTTCTTGGGCTGGGCC | |
| | GATAACGTGGCCCGATCAGTTGGGTCCACCGGAATCTGCTTCAACACT | |
| | GGTGGGGCCTACGGTGTCGGTGCGTATGTTTCAATCTGGAGCAATCTA | |
| | CCTCACCGGAATGTCGGCGATGATGAGGAGTTTTCATTGGCTGGTTTC | |
| | CCGGAAGATCGGAAACTCCGACGTAATCAGCTTCATCGGTTTTTAAGG | |
| | TTTGCTGATGGGTCCGATGAATGGTCGAGGTTTTTCCAACCTCAGATT | |
| | AAATCATCCTTGAATTGTTCTGGGTGGTTGTGTAATTCCATCGAGGAG | |
| | ATTGAACCGTTAGGGTTTCAAGTTTTTAGGAATTTCACGAAATCTCCT | |
| | ATTTGGGGAATTGGTCCTTTGATTATGACATCTTCGAAAAAAGATGAT | |
| | GATGAGAAGGAAGAGGCTTGTTTGAGGTGGTTGAATCAATTTGAAAAT | |
| | GACTCAGTTTTGTACATTTGTTTCGGGTCACAGAATACAGTGACTCCG | |
| | ATTCAGATGATGGAGTTAGCGAAAGGTTTGGAGGAGAGTAAGATCCCG | |
| | TTCTTGTGGGTGATTCGGCCGCCATTCGGGTTCGATTTCAATGGGGAG | |
| | TTTAAACCCGAATGGCTGCCTGAGAAGTTTGAGGAAAGAATGATGGAG | |
| | AAAAAACAGGGGATGTTGGTGAGAGATTGGGTGCCTCAGTTGGATATT | |
| | TTGAGGCATGAGGCTACTGGTGGATTTTTGAGTCATTGTGGTTGGAAT | |
| | TCGGTATTGGAAGGTTTAAGAGAAGGAGTGCCAATACTCGGGTGGCCG | |
| | TTGGCGGCCGAGCAAGCGTATAATTCAAAGATGATGGTGGAGGAAATG | |
| | GGGGTGGCGGTCGAGTTGACGAGGGGGTTGGAAGGGGAGGTGAAGAAA | |
| | GAGTGGGTGAAGAGTGTGGTGGAAATGGTGTTGGATAGAAAGGAGGGG | |
| | AGTTGTGGGTGGGAAATGAAGAAGAAAGCTGTTGAGATTGGGGAGAAA | |
| | TTGAAGGATGCTTGGAAAATTGAGGGAGATTACAAAGGATCTTCTGTT | |
| | AAGGCAATGGATAATTTTGTAGAGTTTATAGTTTCTTGTAGGGGAAAG | |
| | AAATCTAAAGAAGATGCTTGAGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTCAGATGA | |
| | GACCGAGGATGCACATGTGACCGAGGGACACGAA | |
| PL-461 | TGAACACTCTGTGGGTCTCATGCCGAATTCGGATCCGGAGGAAGCCAA | 195 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATAATGCAACCCTAGCAATGATACTTACAATA | |
| | TGGCTCATTTCAATTAATTTCATAAAAATGTTTTTTACTCACCAAAAC | |
| | ACAAAACTTATTCTTCCTCCGGGTCCAAAACCATTACCAATTATTGGC | |
| | AACATTCTCGAAGTTGGGAAAAAACCTCATCGTTCATTCGCAAACCTA | |
| | GCCAAAATCCACGGCCCTTTAATTTCCTTACGTTTAGGAAGTGTTACA | |
| | ACAATTGTCGTATCATCAGCAGAAGTTGCCAAAGAAATGTTCTTAAAA | |
| | AAAGACCAGCCTCTCTCAAACCGTAATGTTCCCAACTCCGTCACTGCT | |
| | GGTGATCACCACAAACTAACCATGTCATGGTTACCCGTATCACCCAAA | |
| | TGGAGAAATTTCCGTAAAATCACAGCCGTTCATTTACTTTCACCTCTA | |
| | AGACTTGATGCATGTCAATCTCTTAGACAAGCCAAGGTGCAACAACTT | |
| | TATCAATACGTACAAGAATGTGCTCAAAAAGGACAATCAATTGATATC | |
| | GGTAAAGCTGCATTTACAACATCGTTAAATCTTTTATCAAAACTATTT | |
| | TTTTCAAAGGAATTAGCTTGCCATAAATCTCATGAATCTCAAGAATTT | |
| | AAACAATTAATTTGGAATATAATGGAAGATATTGGTAAACCTAATTAT | |
| | GCAGATTATTTTCCAATATTAGGATGTATTGATCCATTAGGGATTAGA | |
| | AGAAGATTAGCAGCTAATTTTGATAAATTAATTGCTGTTTTTCAAACT | |
| | ATAATATCTGAAAGGCTTGAGAATAATAAAAACGCAAATGCAACTAAT | |
| | GATGTGCTTGATGTTCTGCTGCAATTGTACAAACAAAAGGAGCTGAGT | |
| | ATGGGTGAAATTAATCATCTACTCGTGGATATTTTTGATGCTGGAACA | |
| | GATACTACTTCAAGCACTTTTGAATGGGTTATGACAGAATTAATTAGA | |
| | AATCCAAATATGATGGAAAAAGCTCAACAAGAAATTCAAGAAGTATTA | |
| | GGTAAAGATAGACAAATTCAAGAATCGGACATTATTAAGTTGCCTTAC | |
| | TTACAAGCCCTTATTAAAGAAACATTACGATTACATCCACCAACTGTA | |
| | TTTCTCTTACCCCGTAAAGCTGATATGGACGTTGAACTATATGGATAT | |
| | GTTGTGCCTAAAGATGCACAAATACTCGTTAATTTGTGGGCTATTGGA | |
| | AGAGATCCTCAAGCATGGGAAAAACCTAATGCTTTTTTACCCGAGAGA | |
| | TTTTTAGGTTCTGATGTTGATGTAAAAGGACGAGATTTCGGACTTTTA | |
| | CCTTTTGGAGCCGGAAAAAGAATATGTCCCGGAATGAATTTGGCTATT | |
| | AGAATGTTGACTTTGATGTTGGCTACTTTACTTCAATTTTTTGATTGG | |
| | AAACTTGAAGAAGGTATGAATCCACAAGATTTAGACATGGATGAAAAA | |
| | TTTGGGATTGCTTTACAAAAGACCAAGCCTCTTGAAATCATTCCATCC | |
| | CGTAGGCATTGAGCTTCTCTAGCTAGAGTCGATCGACAAGCTCGAGTT | |
| | TCTCCATAATAATGTGTGAGTAGTTCCCAGATAAGGGAATTAGGGTTC | |
| | CTATAGGGTTTCGCTCATGTGTTGAGCATATAAGAAACCCTTAGTATG | |
| | TATTTGTATTTGTAAAATACTTCTATCAATAAAATTTCTAATTCCTAA | |
| | AACCAAAATCCAGTACTAAAATCCAGATCGCTACTAGAATTCGAGCTC | |
| | GGAGGAAGCCAACTAAACAAGACCATAACCATGGTGACATTTGACATA | |
| | GTTGTTTACTACTTGCTTGAGCCCCACCCTTGCTTATCGGTTGAACAT | |
| | TACAAGATACACTGCGGGTGGCCTAAGGCACACCGTCCGAAACCGGCA | |
| | AACCAAGCCTGATCGCCGAAATCCAAAATCACTACCGGCAATCTCTAA | |
| | AGTTTATTTCATCCTTATATGACGAGGAAAGAAAAGAAGAGAGAAATA | |
| | ATATCTTAACTTCTAAATCAGTCGCGTCAACTTTCTCGGCTAAGAAAG | |
| | TGAGCACTATCATTTCGCAGACCATGTCATGAGTGCCGACTTGCCATA | |
| | TCTTATTATATTCTTATTTATTTAATTATAATCCCATTGCAATACGTC | |
| | TATTCTATCATGGCCTGCCACTAACGCTCCGTCTAACGTCGTTAAGCC | |
| | ATTGTCATAAGCGGCTGCTCAAAACTCTTCCCGGTGGAGGCGAGGCGT | |
| | TAACGGCGTCTACAAATCTAACGGCCACCAACCATCCAGCCGCCTCTC | |
| | GAAAGCTCCGCTCCGATCGCGGAAATTGCGTGGCGGAGACGAGCGGGC | |
| | TCCTCTCACACGGCCCGGAACCGTCACGGCACGGGTGGGGGATTCCTT | |
| | CCCCAACCCTCCCCACCTCTCCTCCCCCCGTCGCAGCCCATAAATACA | |
| | GGGCCCTCCGCGCCTCTTCCCACAATCTCACATCGTCTCATCGTTCGG | |
| | AGCGCACAACCCCCGGGTTCCAAATCCAAATTGCTCTTCTCGCGACCC | |
| | TCGGCGATCCTTCCCCCGCTTCAAGGTACGGCGATCGTCTCCCCCGTC | |
| | CTCTTGCCCCATCTCCTCGCTCGGCGTGGTTTGGTGGTTCTGCTTGGT | |
| | CTGTGGCTAGGAACTAGGCTGAGGCGTTGACGAAATCATGCTAGATCC | |
| | GCGTGTTTCCTGATCGTGGGTGGCTGGGAGGTGGGGTTTTCGTGTAGA | |
| | TCTGATCGGTTCCGCTGTTTATCCTGTCATGCTCATGTGATTTGTGGG | |
| | GATTTTAGGTCGTTTGTCCGGGAATCGTGGGGTTGCTTCTAGGCTGTT | |
| | CGTAGATGAGATCGTTCTCACGATCTGCTGGGTCGCTGCCTAGGTTCA | |
| | GCTAGGTCTGCCCTGTTTTTGGGTTCGTTTTCGGGATCTGTACGTGCA | |
| | TCTATTATCTGGTTCGATGGTGCTAGCTAGGAACAAACAACTGATTCG | |
| | TCCGATCGATTGTTTTGTTGCCATGTGCAAGGTTAGGTCGTTATCTGA | |
| | TTGCTGTAGATCAGAGTAGAATAAGATCATCACAAGCTAGCTCTTGGG | |
| | CTTATTATGAATCTGCGTTTGTTGCATGATTAAGATGATTATGCTTTT | |
| | TCTTATGCTGCCGTTTGTATATGATGCGGTAGCTTTTAACTGAATAGC | |
| | ACACCTTTCCTGTTTAGTTAGATTAGATTAGATTGCATGATAGATGAG | |
| | GATATATGCTGCTACATCAGTTTGATGATTCTCTGGTACCTCATAATC | |
| | AACTAGCTCATGTGCTTAAATTGAAACTGCATGTGCCACATGATTAAG | |
| | ATGCTAAGATTGGTGAAGATATATACGCTGCTGTTCCTATAGGATCCT | |
| | GTAGCTTTTACCTGGTCAACATGCATCGTCCTGTTATGGATAGATATG | |
| | CATGATAGATGAAGATATGTACTGCTACAATTTGATGATTCTTTTGTG | |
| | CACCTGATGATCATGCATGCTCTTTGCCCTTACTTTGATATACTTGGA | |
| | TGATGGCATGCTTAGTACTAATGATGTGATGAACACACATGACCTGTT | |
| | GGTATGAATATGATGTTGCTGTTTGCTTGTGATGAGTTCTGTTTGTTT | |
| | ACTGCTAGGCACTTACCCTGTTGTCTGGTTCTCTTTTGCAGAATGAAA | |
| | ATGAGTGGAGAAGATAATAGCATGATCAAAGAAACTTTCTTCATAACC | |
| | CATGGAAACCCGATTTTGACCGTAGAAGATACACATCCCTTAAGGCCA | |
| | TTCTTTGAAACATGGAGAGAAAAAATCTTTACGATGAAGCCGAAGGCG | |
| | ATACTTGTGATCTCTGGTCACTGGGAAACTGATCATCCTGCTGTTAAT | |
| | GCTGTTCATATCAATGATACTATCTACGATTTCGATGACTATCCTCAA | |
| | GCCATGTACAAGTTGAAGTATCCAGCTCCAGGATCACCAGACTTGGCT | |
| | AGCAGAGTAGAAGAAATTATGAAAAAATCCGGGTTCGACACAGTGCAC | |
| | ATTGACAAAAAACGTGGGCTTGATCATGGTGCATGGGTGCCTCTTATG | |
| | TACATGTATCCGGAGGCTGACATACCGGTATGTCAGCTCTCCGTCCAG | |
| | CCGAAGTTGGACGGAACGTACCACTACAACTTGGGACGAGCATTAGCG | |
| | CCTCTAAGAGACGAAGGTGTACTCATTATTGGTTCCGGAAGTGCAACA | |
| | CACCCTTTGGACGAAACCCCTCATTATCATGGTGGTGTTGCTCCTTGG | |
| | GCTGCTGACTTTGACTCATGGCTTGACGTAGCTCTCACTAAAGGAAGA | |
| | TTTGAAGAAGTGAATACATATGAAACCAAAGCACCAAACTGGGAATTG | |
| | GCACATCCATTCCCGGAGCATTTTTATCCATTGCATGTCGTGGTTGGG | |
| | GCGGCTGGTGAAAAGTGGAAGGCTGAGCTTATTCATACTAGTTGGGAC | |
| | CATGGTACCTTATGTCATGGCTCCTACAAGTTTACTTCCACCTAAGCT | |
| | TCTCTAGCTAGAGTCGATCGACAAGCTCGAGTTTCTCCATAATAATGT | |
| | GTGAGTAGTTCCCAGATAAGGGAATTAGGGTTCCTATAGGGTTTCGCT | |
| | CATGTGTTGAGCATATAAGAAACCCTTAGTATGTATTTGTATTTGTAA | |
| | AATACTTCTATCAATAAAATTTCTAATTCCTAAAACCAAAATCCAGTA | |
| | CTAAAATCCAGATCGCTTTACGAATTCCCATGGGGAGGAAGCCAACTA | |
| | AACAAGACCATAACCATGGTGACATTTGACATAGTTGTTTACTACTTG | |
| | CTTGAGCCCCACCCTTGCTTATCGGTTGAACATTACAAGATACACTGC | |
| | GGGTGGCCTAAGGCACACCGTCCGAAACCGGCAAACCAAGCCTGATCG | |
| | CCGAAATCCAAAATCACTACCGGCAATCTCTAAAGTTTATTTCATCCT | |
| | TATATGACGAGGAAAGAAAAGAAGAGAGAAATAATATCTTAACTTCTA | |
| | AATCAGTCGCGTCAACTTTCTCGGCTAAGAAAGTGAGCACTATCATTT | |
| | CGCAGACCATGTCATGAGTGCCGACTTGCCATATCTTATTATATTCTT | |
| | ATTTATTTAATTATAATCCCATTGCAATACGTCTATTCTATCATGGCC | |
| | TGCCACTAACGCTCCGTCTAACGTCGTTAAGCCATTGTCATAAGCGGC | |
| | TGCTCAAAACTCTTCCCGGTGGAGGCGAGGCGTTAACGGCGTCTACAA | |
| | ATCTAACGGCCACCAACCATCCAGCCGCCTCTCGAAAGCTCCGCTCCG | |
| | ATCGCGGAAATTGCGTGGCGGAGACGAGCGGGCTCCTCTCACACGGCC | |
| | CGGAACCGTCACGGCACGGGTGGGGGATTCCTTCCCCAACCCTCCCCA | |
| | CCTCTCCTCCCCCCGTCGCAGCCCATAAATACAGGGCCCTCCGCGCCT | |
| | CTTCCCACAATCTCACATCGTCTCATCGTTCGGAGCGCACAACCCCCG | |
| | GGTTCCAAATCCAAATTGCTCTTCTCGCGACCCTCGGCGATCCTTCCC | |
| | CCGCTTCAAGGTACGGCGATCGTCTCCCCCGTCCTCTTGCCCCATCTC | |
| | CTCGCTCGGCGTGGTTTGGTGGTTCTGCTTGGTCTGTGGCTAGGAACT | |
| | AGGCTGAGGCGTTGACGAAATCATGCTAGATCCGCGTGTTTCCTGATC | |
| | GTGGGTGGCTGGGAGGTGGGGTTTTCGTGTAGATCTGATCGGTTCCGC | |
| | TGTTTATCCTGTCATGCTCATGTGATTTGTGGGGATTTTAGGTCGTTT | |
| | GTCCGGGAATCGTGGGGTTGCTTCTAGGCTGTTCGTAGATGAGATCGT | |
| | TCTCACGATCTGCTGGGTCGCTGCCTAGGTTCAGCTAGGTCTGCCCTG | |
| | TTTTTGGGTTCGTTTTCGGGATCTGTACGTGCATCTATTATCTGGTTC | |
| | GATGGTGCTAGCTAGGAACAAACAACTGATTCGTCCGATCGATTGTTT | |
| | TGTTGCCATGTGCAAGGTTAGGTCGTTATCTGATTGCTGTAGATCAGA | |
| | GTAGAATAAGATCATCACAAGCTAGCTCTTGGGCTTATTATGAATCTG | |
| | CGTTTGTTGCATGATTAAGATGATTATGCTTTTTCTTATGCTGCCGTT | |
| | TGTATATGATGCGGTAGCTTTTAACTGAATAGCACACCTTTCCTGTTT | |
| | AGTTAGATTAGATTAGATTGCATGATAGATGAGGATATATGCTGCTAC | |
| | ATCAGTTTGATGATTCTCTGGTACCTCATAATCAACTAGCTCATGTGC | |
| | TTAAATTGAAACTGCATGTGCCACATGATTAAGATGCTAAGATTGGTG | |
| | AAGATATATACGCTGCTGTTCCTATAGGATCCTGTAGCTTTTACCTGG | |
| | TCAACATGCATCGTCCTGTTATGGATAGATATGCATGATAGATGAAGA | |
| | TATGTACTGCTACAATTTGATGATTCTTTTGTGCACCTGATGATCATG | |
| | CATGCTCTTTGCCCTTACTTTGATATACTTGGATGATGGCATGCTTAG | |
| | TACTAATGATGTGATGAACACACATGACCTGTTGGTATGAATATGATG | |
| | TTGCTGTTTGCTTGTGATGAGTTCTGTTTGTTTACTGCTAGGCACTTA | |
| | CCCTGTTGTCTGGTTCTCTTTTGCAGAATGGATTCATCAAAAACAAAG | |
| | CACATTATCCTCTTGCCATTTCTAGCGCAAGGTCATCTCCGTCCATTT | |
| | CTGCACTTAGCCAATTTCCTCAGATCCCACACTCCTTTCACCCTTTCT | |
| | ATCCTTACTACTCCTCTCAATGCCGCCAATCTCCGCCGTCAATCCAAC | |
| | AACATTAACATCTACGATCTACCCTTTAACAGCACCGATCATAATCTA | |
| | CCACCAAACACAGAAAACACTGAAAAACTCCCATTAACTTCAATAATC | |
| | TCTCTGTTTTATGCATCCACTTCGCTCCAGCCTCACGTACGCAACCAC | |
| | CTTACTCGCCATCACCTTGATAACCCACCCATTTGTATCATCTTTGAT | |
| | ATTTTCTTGGGCTGGGCTGATAACTTGGCCCGATCTATTGGATCTACC | |
| | GGAGTTTGTTTCAACACTGGTGGGGCCTACGGTTTGGCCGCTTACATG | |
| | TCGATTTGGATGCATTTGCCTCATAGAAATGTTCCAGACAATGTCGAG | |
| | TTTTCCCTTCCGGAATTCCCTGAGAATCGGAAATTTAAACGTAACCAA | |
| | CTTCACAGGTTTTTAAGATTTGCTGATGGAACCGATGATTGGTCGGGT | |
| | TTTTTTCAACCCCAGATAAAGTACTCCAGGAATTGTTCTGCGTGGTTG | |
| | TGTAATTCAATTGAGGAAATTGAGCCATTAGGGTTTGAGGTTTTAAGA | |
| | GCAATGTTGAAAGTACCAGTTTGGGGAATTGGTCCTTTAGTCAAGACT | |
| | GAAAATAGTTGTGATGAAGATGATGAACAAGGTTGTGTTGAGTGGTTA | |
| | AACCAATTTGAGAAAGGATCTGTTTTGTACATATCATTTGGGTCACAG | |
| | AATACAGTTACCCCGATCCAAATGATGGAATTAGCAAAAGGATTGGAG | |
| | GAAAGTGGGGCGAAATTTTTGTGGGTGATTCGTCCCCCTTTCGGGTTT | |
| | GATATCAATGGAGAGTTCAAACCCGAATGGCTGCCGGAGGGATTTGAG | |
| | AAGAGAGTAATGGAAAGGAAGCAGGGGAAGCTAGTAAAGAAATGGGGG | |
| | CCTCAAATGGAGATATTGAGGAATAAGGCGACGGGGGCGTTTTTGAGT | |
| | CACTGTGGGTGGAATTCGTTGATCGAAGCGTTAAGCGAAGGTGTTCCG | |
| | ATAATAGGGTGGCCATTGGCTGCGGAGCAAGCGTATAATTCAAAGATG | |
| | TTAGTGGAAGAGATGGGGGTTGCGGTCGAGTTAACGAGGGGATTGGAA | |
| | GGGGAGTTGAGTAGCGAAGGGGTGAAGAAGGTGGTGGAGATGGTTATG | |
| | GATAGGGAAGAAGGGAGTTTTGGGTGTGAGATGAAGAAGGCGGCAGTT | |
| | GTAATTGGACAGAAATTGAGGGATGCAATGAAAGTGGAAGGTGATTAT | |
| | AGAGGGTGTTCTCTTAGATCTCTTGATGAGTTTGTAGAGTTCATTGTT | |
| | TCTCGTAAGGCTACTTTGAGCTAGGCTTCTCTAGCTAGAGTCGATCGA | |
| | CAAGCTCGAGTTTCTCCATAATAATGTGTGAGTAGTTCCCAGATAAGG | |
| | GAATTAGGGTTCCTATAGGGTTTCGCTCATGTGTTGAGCATATAAGAA | |
| | ACCCTTAGTATGTATTTGTATTTGTAAAATACTTCTATCAATAAAATT | |
| | TCTAATTCCTAAAACCAAAATCCAGTACTAAAATCCAGATCGCTCAGA | |
| | TGAGACCGAGGATGCACATGTGACCGAGGGACACGAA | |
| PL-462 | TGAACACTCTGTGGGTCTCATGCCGAATTCGGATCCGGAGGAAGCCAA | 196 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATCATGCAACACTAGCAATGATACTTGCTATT | |
| | TGGTTTGTTGTTTTTCATTTCATTAAAATGTTATTCACTAGCCAAACT | |
| | ACCAAACTTCTTCCCCCCGGTCCTAAACCATTGCCACTAATCGGAAAC | |
| | ATCCTTGAAGTTGGTGAGAAACCTCACCAGTCATTTGCTAATCTTGCT | |
| | AAGATTCATGGTCCATTGATATCATTACGTCTAGGAAGTGTCACAACT | |
| | ATTGTTGTATCCTCGGCTGAAGTAGCCAAAGAAATGTTCTTAAAAAAA | |
| | GACCACCCACTTTCTAACCGCACTGTTCCTAATTCTGTCACTGCTGGG | |
| | GATCACCATAAGCTGACTATGTCGTGGTTGCCTGTTTCTCCAAAGTGG | |
| | CGGAACTTTAGAAAAATCACCGCCGTTCATTTACTTTCTCCTCAAAGA | |
| | CTTGATGCTTGCCAAACCCTAAGACATGCCAAAGTGCAACAGCTTTTC | |
| | CAATATGTACAAGAATGTGCACAAAAAGGACAAGCTGTTGATATTGGC | |
| | AAGGCAGCATTTACAACCTCCCTTAATTTGTTATCAAAATTATTCTTT | |
| | TCGGTCGAATTAGCCCACCATAAATCCCATACATCTCAACAATTCAAA | |
| | GAACTTATATGGAATATTATGGAAGATATTGGCAAACCTAACTACGCT | |
| | GATTATTTTCCAATCTTAGGATGCTTAGACCCATCAGGAATTCGACGT | |
| | CGATTGGCGTCTAATTTTGACAAGTTGATTGCAGTTTTTCAAAGCATA | |
| | ATATGTCAAAGGATTGGCAACGGCCAGGATTCTGCTTCGACTAAGACG | |
| | ACCGATGATGTGCTAGACATTCTTCTTGACCTCCACAAACAAAAAGAG | |
| | CTCAGTATGGGCGAGATAAATCATCTGCTCGTGGATATTTTTGATGCT | |
| | GGGACTGACACTACATCAAGTACTTTTGAATGGGTAATGGCAGAGTTA | |
| | ATTCGAAATCCTAAGATGATGGAAAAAGCTCAAGAAGAAATTGAGCAA | |
| | GTCTTGGGCAAGGATAGACAAATTCAAGAATCAGACATTATTAAGCTA | |
| | CCTTACTTACAAGCCATTATCAAAGAAACATTGCGGCTACACCCACCA | |
| | ACTGTATTTCTCTTGCCTCGTAAAGCTGATAGTGATGTTGAATTATAT | |
| | GGCTATGTTGTGCCGAAAGATGCACAAATACTTGTTAATTTATGGGCC | |
| | ATTGGAAGGGACCCTCAAGCGTGGGTGAAACCTGACGTGTTTTTACCT | |
| | GAGAGGTTTTTAGGATCCGAAATTGATGTGAAAGGAAGAGATTTTGGA | |
| | CTCTTACCTTTTGGAGCTGGAAGGAGAATATGCCCCGGGATGAATTTG | |
| | GCTATTAGAATGTTAACTTTGATGTTAGCTACGCTTCTTCAATTCTTC | |
| | AATTGGAAGCTTGAAGAAGGTATGAAAGCAGAAGATCTAGACATGGAT | |
| | GAGAAATTTGGGATTGCCTTACAAAAGACTAAACCTCTTCAGATTATT | |
| | CCCGTTCTTAGGTATTGAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGGTAGAAAAAGAAATGAATTTCAATGAAACCTTTTTTATAACCCAT | |
| | GGAAACCCAATTTTAACAGTTGAAGATTCTCATCCTTTAAGACCTTTT | |
| | TTTGAAACTTGGAAACAAAATATATTTTCTAAGAAACCAAAGGCTATT | |
| | CTTGTGATTTCAGGTCATTGGGAAACTGATTTTCCTTCTGTTAATGCT | |
| | GTTGATATCAATGATACTATCCATGATTTTGATGATTACCCTGATGCT | |
| | ATGTACCAGTTGAAATATCCAGCTCCAGGGTCAATAGAAATGGCAAAA | |
| | AAAGTGGAAGAAATACTGACAAAATCAGGTTACAAAACAATAAACATG | |
| | GACAAAAAACGAGGACTAGATCATGGTGCATGGGTACCATTAATGTAC | |
| | ATGTATCCCCAAGCAGACATCCCAGTTTGTCAACTATCAGTTCAACCC | |
| | AAGTTAAGTGGAACATACCATTATAACTTGGGAAGAGCTTTGGCTCCT | |
| | TTGAAACAAGAAGGTGTACTTATAATTGGTTCCGGAAGTGCTACTCAT | |
| | CCTTTGGATGAAACCCCACATTTTCATAATGGTGTTGCACCTTGGGCT | |
| | CATGAATTTCATTCTTGGCTTCATTCTGCTCTTATAAATCGAAGGATT | |
| | GAAGAAGTGAACACATATGAAAGCAAAGCACCAAATTGGGAATTAGCA | |
| | CATCCATTCCCAGAACATTTTTATCCCTTGCACATTGTCATGGGTGCA | |
| | GCAGGAGATAATTGGAAGGCAGAGCTTATTCATAGCAGTTGGGACCAT | |
| | GGCACCTTATATCATGGTGCCTACAAGTTCACTTCCAACTAAGCTTCT | |
| | CTAGCTAGAGTCGATCGACAAGCTCGAGTTTCTCCATAATAATGTGTG | |
| | AGTAGTTCCCAGATAAGGGAATTAGGGTTCCTATAGGGTTTCGCTCAT | |
| | GTGTTGAGCATATAAGAAACCCTTAGTATGTATTTGTATTTGTAAAAT | |
| | ACTTCTATCAATAAAATTTCTAATTCCTAAAACCAAAATCCAGTACTA | |
| | AAATCCAGATCGCTTTACGAATTCCCATGGGGAGGAAGCCAACTAAAC | |
| | AAGACCATAACCATGGTGACATTTGACATAGTTGTTTACTACTTGCTT | |
| | GAGCCCCACCCTTGCTTATCGGTTGAACATTACAAGATACACTGCGGG | |
| | TGGCCTAAGGCACACCGTCCGAAACCGGCAAACCAAGCCTGATCGCCG | |
| | AAATCCAAAATCACTACCGGCAATCTCTAAAGTTTATTTCATCCTTAT | |
| | ATGACGAGGAAAGAAAAGAAGAGAGAAATAATATCTTAACTTCTAAAT | |
| | CAGTCGCGTCAACTTTCTCGGCTAAGAAAGTGAGCACTATCATTTCGC | |
| | AGACCATGTCATGAGTGCCGACTTGCCATATCTTATTATATTCTTATT | |
| | TATTTAATTATAATCCCATTGCAATACGTCTATTCTATCATGGCCTGC | |
| | CACTAACGCTCCGTCTAACGTCGTTAAGCCATTGTCATAAGCGGCTGC | |
| | TCAAAACTCTTCCCGGTGGAGGCGAGGCGTTAACGGCGTCTACAAATC | |
| | TAACGGCCACCAACCATCCAGCCGCCTCTCGAAAGCTCCGCTCCGATC | |
| | GCGGAAATTGCGTGGCGGAGACGAGCGGGCTCCTCTCACACGGCCCGG | |
| | AACCGTCACGGCACGGGTGGGGGATTCCTTCCCCAACCCTCCCCACCT | |
| | CTCCTCCCCCCGTCGCAGCCCATAAATACAGGGCCCTCCGCGCCTCTT | |
| | CCCACAATCTCACATCGTCTCATCGTTCGGAGCGCACAACCCCCGGGT | |
| | TCCAAATCCAAATTGCTCTTCTCGCGACCCTCGGCGATCCTTCCCCCG | |
| | CTTCAAGGTACGGCGATCGTCTCCCCCGTCCTCTTGCCCCATCTCCTC | |
| | GCTCGGCGTGGTTTGGTGGTTCTGCTTGGTCTGTGGCTAGGAACTAGG | |
| | CTGAGGCGTTGACGAAATCATGCTAGATCCGCGTGTTTCCTGATCGTG | |
| | GGTGGCTGGGAGGTGGGGTTTTCGTGTAGATCTGATCGGTTCCGCTGT | |
| | TTATCCTGTCATGCTCATGTGATTTGTGGGGATTTTAGGTCGTTTGTC | |
| | CGGGAATCGTGGGGTTGCTTCTAGGCTGTTCGTAGATGAGATCGTTCT | |
| | CACGATCTGCTGGGTCGCTGCCTAGGTTCAGCTAGGTCTGCCCTGTTT | |
| | TTGGGTTCGTTTTCGGGATCTGTACGTGCATCTATTATCTGGTTCGAT | |
| | GGTGCTAGCTAGGAACAAACAACTGATTCGTCCGATCGATTGTTTTGT | |
| | TGCCATGTGCAAGGTTAGGTCGTTATCTGATTGCTGTAGATCAGAGTA | |
| | GAATAAGATCATCACAAGCTAGCTCTTGGGCTTATTATGAATCTGCGT | |
| | TTGTTGCATGATTAAGATGATTATGCTTTTTCTTATGCTGCCGTTTGT | |
| | ATATGATGCGGTAGCTTTTAACTGAATAGCACACCTTTCCTGTTTAGT | |
| | TAGATTAGATTAGATTGCATGATAGATGAGGATATATGCTGCTACATC | |
| | AGTTTGATGATTCTCTGGTACCTCATAATCAACTAGCTCATGTGCTTA | |
| | AATTGAAACTGCATGTGCCACATGATTAAGATGCTAAGATTGGTGAAG | |
| | ATATATACGCTGCTGTTCCTATAGGATCCTGTAGCTTTTACCTGGTCA | |
| | ACATGCATCGTCCTGTTATGGATAGATATGCATGATAGATGAAGATAT | |
| | GTACTGCTACAATTTGATGATTCTTTTGTGCACCTGATGATCATGCAT | |
| | GCTCTTTGCCCTTACTTTGATATACTTGGATGATGGCATGCTTAGTAC | |
| | TAATGATGTGATGAACACACATGACCTGTTGGTATGAATATGATGTTG | |
| | CTGTTTGCTTGTGATGAGTTCTGTTTGTTTACTGCTAGGCACTTACCC | |
| | TGTTGTCTGGTTCTCTTTTGCAGAATGGATTCATCAAAAACAAAGCAC | |
| | ATTATCCTCTTGCCATTTCTAGCGCAAGGTCATCTCCGTCCATTTCTG | |
| | CACTTAGCCAATTTCCTCAGATCCCACACTCCTTTCACCCTTTCTATC | |
| | CTTACTACTCCTCTCAATGCCGCCAATCTCCGCCGTCAATCCAACAAC | |
| | ATTAACATCTACGATCTACCCTTTAACAGCACCGATCATAATCTACCA | |
| | CCAAACACAGAAAACACTGAAAAACTCCCATTAACTTCAATAATCTCT | |
| | CTGTTTTATGCATCCACTTCGCTCCAGCCTCACGTACGCAACCACCTT | |
| | ACTCGCCATCACCTTGATAACCCACCCATTTGTATCATCTTTGATATT | |
| | TTCTTGGGCTGGGCTGATAACTTGGCCCGATCTATTGGATCTACCGGA | |
| | GTTTGTTTCAACACTGGTGGGGCCTACGGTTTGGCCGCTTACATGTCG | |
| | ATTTGGATGCATTTGCCTCATAGAAATGTTCCAGACAATGTCGAGTTT | |
| | TCCCTTCCGGAATTCCCTGAGAATCGGAAATTTAAACGTAACCAACTT | |
| | CACAGGTTTTTAAGATTTGCTGATGGAACCGATGATTGGTCGGGTTTT | |
| | TTTCAACCCCAGATAAAGTACTCCAGGAATTGTTCTGCGTGGTTGTGT | |
| | AATTCAATTGAGGAAATTGAGCCATTAGGGTTTGAGGTTTTAAGAGCA | |
| | ATGTTGAAAGTACCAGTTTGGGGAATTGGTCCTTTAGTCAAGACTGAA | |
| | AATAGTTGTGATGAAGATGATGAACAAGGTTGTGTTGAGTGGTTAAAC | |
| | CAATTTGAGAAAGGATCTGTTTTGTACATATCATTTGGGTCACAGAAT | |
| | ACAGTTACCCCGATCCAAATGATGGAATTAGCAAAAGGATTGGAGGAA | |
| | AGTGGGGCGAAATTTTTGTGGGTGATTCGTCCCCCTTTCGGGTTTGAT | |
| | ATCAATGGAGAGTTCAAACCCGAATGGCTGCCGGAGGGATTTGAGAAG | |
| | AGAGTAATGGAAAGGAAGCAGGGGAAGCTAGTAAAGAAATGGGGGCCT | |
| | CAAATGGAGATATTGAGGAATAAGGCGACGGGGGCGTTTTTGAGTCAC | |
| | TGTGGGTGGAATTCGTTGATCGAAGCGTTAAGCGAAGGTGTTCCGATA | |
| | ATAGGGTGGCCATTGGCTGCGGAGCAAGCGTATAATTCAAAGATGTTA | |
| | GTGGAAGAGATGGGGGTTGCGGTCGAGTTAACGAGGGGATTGGAAGGG | |
| | GAGTTGAGTAGCGAAGGGGTGAAGAAGGTGGTGGAGATGGTTATGGAT | |
| | AGGGAAGAAGGGAGTTTTGGGTGTGAGATGAAGAAGGCGGCAGTTGTA | |
| | ATTGGACAGAAATTGAGGGATGCAATGAAAGTGGAAGGTGATTATAGA | |
| | GGGTGTTCTCTTAGATCTCTTGATGAGTTTGTAGAGTTCATTGTTTCT | |
| | CGTAAGGCTACTTTGAGCTAGGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTCAGATGA | |
| | GACCGAGGATGCACATGTGACCGAGGGACACGAA | |
| PL-463 | TTCGTGTCCCTCGGTCACATGTGCATCCTCGGTCTCATCTGAGCGATC | 197 |
| construct | TGGATTTTAGTACTGGATTTTGGTTTTAGGAATTAGAAATTTTATTGA | |
| | TAGAAGTATTTTACAAATACAAATACATACTAAGGGTTTCTTATATGC | |
| | TCAACACATGAGCGAAACCCTATAGGAACCCTAATTCCCTTATCTGGG | |
| | AACTACTCACACATTATTATGGAGAAACTCGAGCTTGTCGATCGACTC | |
| | TAGCTAGAGAAGCTCAAGCATCTTCTTTAGATTTCTTTCCCCTACAAG | |
| | AAACTATAAACTCTACAAAATTATCCATTGCCTTAACAGAAGATCCTT | |
| | TGTAATCTCCCTCAATTTTCCAAGCATCCTTCAATTTCTCCCCAATCT | |
| | CAACAGCTTTCTTCTTCATTTCCCACCCACAACTCCCCTCCTTTCTAT | |
| | CCAACACCATTTCCACCACACTCTTCACCCACTCTTTCTTCACCTCCC | |
| | CTTCCAACCCCCTCGTCAACTCGACCGCCACCCCCATTTCCTCCACCA | |
| | TCATCTTTGAATTATACGCTTGCTCGGCCGCCAACGGCCACCCGAGTA | |
| | TTGGCACTCCTTCTCTTAAACCTTCCAATACCGAATTCCAACCACAAT | |
| | GACTCAAAAATCCACCAGTAGCCTCATGCCTCAAAATATCCAACTGAG | |
| | GCACCCAATCTCTCACCAACATCCCCTGTTTTTTCTCCATCATTCTTT | |
| | CCTCAAACTTCTCAGGCAGCCATTCGGGTTTAAACTCCCCATTGAAAT | |
| | CGAACCCGAATGGCGGCCGAATCACCCACAAGAACGGGATCTTACTCT | |
| | CCTCCAAACCTTTCGCTAACTCCATCATCTGAATCGGAGTCACTGTAT | |
| | TCTGTGACCCGAAACAAATGTACAAAACTGAGTCATTTTCAAATTGAT | |
| | TCAACCACCTCAAACAAGCCTCTTCCTTCTCATCATCATCTTTTTTCG | |
| | AAGATGTCATAATCAAAGGACCAATTCCCCAAATAGGAGATTTCGTGA | |
| | AATTCCTAAAAACTTGAAACCCTAACGGTTCAATCTCCTCGATGGAAT | |
| | TACACAACCACCCAGAACAATTCAAGGATGATTTAATCTGAGGTTGGA | |
| | AAAACCTCGACCATTCATCGGACCCATCAGCAAACCTTAAAAACCGAT | |
| | GAAGCTGATTACGTCGGAGTTTCCGATCTTCCGGGAAACCAGCCAATG | |
| | AAAACTCCTCATCATCGCCGACATTCCGGTGAGGTAGATTGCTCCAGA | |
| | TTGAAACATACGCACCGACACCGTAGGCCCCACCAGTGTTGAAGCAGA | |
| | TTCCGGTGGACCCAACTGATCGGGCCACGTTATCGGCCCAGCCCAAGA | |
| | ACACGTCGAAGATTATGCACACGGGTGGGTCGTGCAGGTGGTGGCGCG | |
| | TGAGGTAGTCACGTACATGAGGTTCGAGTGAAGTGGATGCATGGAAGA | |
| | GTTTGATGATGGAGGTTAATGAGAGTTTATCGGTATTTTCGGTGTTGG | |
| | GTGGGACACCGTGGTCGGTGCTAGTGAAGGGGAGTTCGATGATGTTAA | |
| | GGTTGATGTTGAAGTTGTCGGATTGACGGCGGAGATTAGCGGCGTTTA | |
| | AAGGGGTAGTGAGGAGAGAGAGATTGAAGGGTGTTAGAGAGAGAAGGC | |
| | GGTGAGCGAGGTGGAGGAATGGTCGGAGATGGCCTTGTGCTAAAAATG | |
| | GGAGGAGGATTATGTGTTGTTGTTCTTGTTCTTTTGTTGATGACTCCA | |
| | TTCTGCAAAAGAGAACCAGACAACAGGGTAAGTGCCTAGCAGTAAACA | |
| | AACAGAACTCATCACAAGCAAACAGCAACATCATATTCATACCAACAG | |
| | GTCATGTGTGTTCATCACATCATTAGTACTAAGCATGCCATCATCCAA | |
| | GTATATCAAAGTAAGGGCAAAGAGCATGCATGATCATCAGGTGCACAA | |
| | AAGAATCATCAAATTGTAGCAGTACATATCTTCATCTATCATGCATAT | |
| | CTATCCATAACAGGACGATGCATGTTGACCAGGTAAAAGCTACAGGAT | |
| | CCTATAGGAACAGCAGCGTATATATCTTCACCAATCTTAGCATCTTAA | |
| | TCATGTGGCACATGCAGTTTCAATTTAAGCACATGAGCTAGTTGATTA | |
| | TGAGGTACCAGAGAATCATCAAACTGATGTAGCAGCATATATCCTCAT | |
| | CTATCATGCAATCTAATCTAATCTAACTAAACAGGAAAGGTGTGCTAT | |
| | TCAGTTAAAAGCTACCGCATCATATACAAACGGCAGCATAAGAAAAAG | |
| | CATAATCATCTTAATCATGCAACAAACGCAGATTCATAATAAGCCCAA | |
| | GAGCTAGCTTGTGATGATCTTATTCTACTCTGATCTACAGCAATCAGA | |
| | TAACGACCTAACCTTGCACATGGCAACAAAACAATCGATCGGACGAAT | |
| | CAGTTGTTTGTTCCTAGCTAGCACCATCGAACCAGATAATAGATGCAC | |
| | GTACAGATCCCGAAAACGAACCCAAAAACAGGGCAGACCTAGCTGAAC | |
| | CTAGGCAGCGACCCAGCAGATCGTGAGAACGATCTCATCTACGAACAG | |
| | CCTAGAAGCAACCCCACGATTCCCGGACAAACGACCTAAAATCCCCAC | |
| | AAATCACATGAGCATGACAGGATAAACAGCGGAACCGATCAGATCTAC | |
| | ACGAAAACCCCACCTCCCAGCCACCCACGATCAGGAAACACGCGGATC | |
| | TAGCATGATTTCGTCAACGCCTCAGCCTAGTTCCTAGCCACAGACCAA | |
| | GCAGAACCACCAAACCACGCCGAGCGAGGAGATGGGGCAAGAGGACGG | |
| | GGGAGACGATCGCCGTACCTTGAAGCGGGGGAAGGATCGCCGAGGGTC | |
| | GCGAGAAGAGCAATTTGGATTTGGAACCCGGGGGTTGTGCGCTCCGAA | |
| | CGATGAGACGATGTGAGATTGTGGGAAGAGGCGCGGAGGGCCCTGTAT | |
| | TTATGGGCTGCGACGGGGGGAGGAGAGGTGGGGAGGGTTGGGGAAGGA | |
| | ATCCCCCACCCGTGCCGTGACGGTTCCGGGCCGTGTGAGAGGAGCCCG | |
| | CTCGTCTCCGCCACGCAATTTCCGCGATCGGAGCGGAGCTTTCGAGAG | |
| | GCGGCTGGATGGTTGGTGGCCGTTAGATTTGTAGACGCCGTTAACGCC | |
| | TCGCCTCCACCGGGAAGAGTTTTGAGCAGCCGCTTATGACAATGGCTT | |
| | AACGACGTTAGACGGAGCGTTAGTGGCAGGCCATGATAGAATAGACGT | |
| | ATTGCAATGGGATTATAATTAAATAAATAAGAATATAATAAGATATGG | |
| | CAAGTCGGCACTCATGACATGGTCTGCGAAATGATAGTGCTCACTTTC | |
| | TTAGCCGAGAAAGTTGACGCGACTGATTTAGAAGTTAAGATATTATTT | |
| | CTCTCTTCTTTTCTTTCCTCGTCATATAAGGATGAAATAAACTTTAGA | |
| | GATTGCCGGTAGTGATTTTGGATTTCGGCGATCAGGCTTGGTTTGCCG | |
| | GTTTCGGACGGTGTGCCTTAGGCCACCCGCAGTGTATCTTGTAATGTT | |
| | CAACCGATAAGCAAGGGTGGGGCTCAAGCAAGTAGTAAACAACTATGT | |
| | CAAATGTCACCATGGTTATGGTCTTGTTTAGTTGGCTTCCTCCCCATG | |
| | GGAATTCGTAAAGCGATCTGGATTTTAGTACTGGATTTTGGTTTTAGG | |
| | AATTAGAAATTTTATTGATAGAAGTATTTTACAAATACAAATACATAC | |
| | TAAGGGTTTCTTATATGCTCAACACATGAGCGAAACCCTATAGGAACC | |
| | CTAATTCCCTTATCTGGGAACTACTCACACATTATTATGGAGAAACTC | |
| | GAGCTTGTCGATCGACTCTAGCTAGAGAAGCTTAGTTGGAAGTGAACT | |
| | TGTAGGCACCATGATATAAGGTGCCATGGTCCCAACTGCTATGAATAA | |
| | GCTCTGCCTTCCAATTATCTCCTGCTGCACCCATGACAATGTGCAAGG | |
| | GATAAAAATGTTCTGGGAATGGATGTGCTAATTCCCAATTTGGTGCTT | |
| | TGCTTTCATATGTGTTCACTTCTTCAATCCTTCGATTTATAAGAGCAG | |
| | AATGAAGCCAAGAATGAAATTCATGAGCCCAAGGTGCAACACCATTAT | |
| | GAAAATGTGGGGTTTCATCCAAAGGATGAGTAGCACTTCCGGAACCAA | |
| | TTATAAGTACACCTTCTTGTTTCAAAGGAGCCAAAGCTCTTCCCAAGT | |
| | TATAATGGTATGTTCCACTTAACTTGGGTTGAACTGATAGTTGACAAA | |
| | CTGGGATGTCTGCTTGGGGATACATGTACATTAATGGTACCCATGCAC | |
| | CATGATCTAGTCCTCGTTTTTTGTCCATGTTTATTGTTTTGTAACCTG | |
| | ATTTTGTCAGTATTTCTTCCACTTTTTTTGCCATTTCTATTGACCCTG | |
| | GAGCTGGATATTTCAACTGGTACATAGCATCAGGGTAATCATCAAAAT | |
| | CATGGATAGTATCATTGATATCAACAGCATTAACAGAAGGAAAATCAG | |
| | TTTCCCAATGACCTGAAATCACAAGAATAGCCTTTGGTTTCTTAGAAA | |
| | ATATATTTTGTTTCCAAGTTTCAAAAAAAGGTCTTAAAGGATGAGAAT | |
| | CTTCAACTGTTAAAATTGGGTTTCCATGGGTTATAAAAAAGGTTTCAT | |
| | TGAAATTCATTTCTTTTTCTACCATTCTGCAAAAGAGAACCAGACAAC | |
| | AGGGTAAGTGCCTAGCAGTAAACAAACAGAACTCATCACAAGCAAACA | |
| | GCAACATCATATTCATACCAACAGGTCATGTGTGTTCATCACATCATT | |
| | AGTACTAAGCATGCCATCATCCAAGTATATCAAAGTAAGGGCAAAGAG | |
| | CATGCATGATCATCAGGTGCACAAAAGAATCATCAAATTGTAGCAGTA | |
| | CATATCTTCATCTATCATGCATATCTATCCATAACAGGACGATGCATG | |
| | TTGACCAGGTAAAAGCTACAGGATCCTATAGGAACAGCAGCGTATATA | |
| | TCTTCACCAATCTTAGCATCTTAATCATGTGGCACATGCAGTTTCAAT | |
| | TTAAGCACATGAGCTAGTTGATTATGAGGTACCAGAGAATCATCAAAC | |
| | TGATGTAGCAGCATATATCCTCATCTATCATGCAATCTAATCTAATCT | |
| | AACTAAACAGGAAAGGTGTGCTATTCAGTTAAAAGCTACCGCATCATA | |
| | TACAAACGGCAGCATAAGAAAAAGCATAATCATCTTAATCATGCAACA | |
| | AACGCAGATTCATAATAAGCCCAAGAGCTAGCTTGTGATGATCTTATT | |
| | CTACTCTGATCTACAGCAATCAGATAACGACCTAACCTTGCACATGGC | |
| | AACAAAACAATCGATCGGACGAATCAGTTGTTTGTTCCTAGCTAGCAC | |
| | CATCGAACCAGATAATAGATGCACGTACAGATCCCGAAAACGAACCCA | |
| | AAAACAGGGCAGACCTAGCTGAACCTAGGCAGCGACCCAGCAGATCGT | |
| | GAGAACGATCTCATCTACGAACAGCCTAGAAGCAACCCCACGATTCCC | |
| | GGACAAACGACCTAAAATCCCCACAAATCACATGAGCATGACAGGATA | |
| | AACAGCGGAACCGATCAGATCTACACGAAAACCCCACCTCCCAGCCAC | |
| | CCACGATCAGGAAACACGCGGATCTAGCATGATTTCGTCAACGCCTCA | |
| | GCCTAGTTCCTAGCCACAGACCAAGCAGAACCACCAAACCACGCCGAG | |
| | CGAGGAGATGGGGCAAGAGGACGGGGGAGACGATCGCCGTACCTTGAA | |
| | GCGGGGGAAGGATCGCCGAGGGTCGCGAGAAGAGCAATTTGGATTTGG | |
| | AACCCGGGGGTTGTGCGCTCCGAACGATGAGACGATGTGAGATTGTGG | |
| | GAAGAGGCGCGGAGGGCCCTGTATTTATGGGCTGCGACGGGGGGAGGA | |
| | GAGGTGGGGAGGGTTGGGGAAGGAATCCCCCACCCGTGCCGTGACGGT | |
| | TCCGGGCCGTGTGAGAGGAGCCCGCTCGTCTCCGCCACGCAATTTCCG | |
| | CGATCGGAGCGGAGCTTTCGAGAGGCGGCTGGATGGTTGGTGGCCGTT | |
| | AGATTTGTAGACGCCGTTAACGCCTCGCCTCCACCGGGAAGAGTTTTG | |
| | AGCAGCCGCTTATGACAATGGCTTAACGACGTTAGACGGAGCGTTAGT | |
| | GGCAGGCCATGATAGAATAGACGTATTGCAATGGGATTATAATTAAAT | |
| | AAATAAGAATATAATAAGATATGGCAAGTCGGCACTCATGACATGGTC | |
| | TGCGAAATGATAGTGCTCACTTTCTTAGCCGAGAAAGTTGACGCGACT | |
| | GATTTAGAAGTTAAGATATTATTTCTCTCTTCTTTTCTTTCCTCGTCA | |
| | TATAAGGATGAAATAAACTTTAGAGATTGCCGGTAGTGATTTTGGATT | |
| | TCGGCGATCAGGCTTGGTTTGCCGGTTTCGGACGGTGTGCCTTAGGCC | |
| | ACCCGCAGTGTATCTTGTAATGTTCAACCGATAAGCAAGGGTGGGGCT | |
| | CAAGCAAGTAGTAAACAACTATGTCAAATGTCACCATGGTTATGGTCT | |
| | TGTTTAGTTGGCTTCCTCCGAGCTCGAATTCTAGTAGCGATCTGGATT | |
| | TTAGTACTGGATTTTGGTTTTAGGAATTAGAAATTTTATTGATAGAAG | |
| | TATTTTACAAATACAAATACATACTAAGGGTTTCTTATATGCTCAACA | |
| | CATGAGCGAAACCCTATAGGAACCCTAATTCCCTTATCTGGGAACTAC | |
| | TCACACATTATTATGGAGAAACTCGAGCTTGTCGATCGACTCTAGCTA | |
| | GAGAAGCTCAATACCTAAGAACGGGAATAATCTGAAGAGGTTTAGTCT | |
| | TTTGTAAGGCAATCCCAAATTTCTCATCCATGTCTAGATCTTCTGCTT | |
| | TCATACCTTCTTCAAGCTTCCAATTGAAGAATTGAAGAAGCGTAGCTA | |
| | ACATCAAAGTTAACATTCTAATAGCCAAATTCATCCCGGGGCATATTC | |
| | TCCTTCCAGCTCCAAAAGGTAAGAGTCCAAAATCTCTTCCTTTCACAT | |
| | CAATTTCGGATCCTAAAAACCTCTCAGGTAAAAACACGTCAGGTTTCA | |
| | CCCACGCTTGAGGGTCCCTTCCAATGGCCCATAAATTAACAAGTATTT | |
| | GTGCATCTTTCGGCACAACATAGCCATATAATTCAACATCACTATCAG | |
| | CTTTACGAGGCAAGAGAAATACAGTTGGTGGGTGTAGCCGCAATGTTT | |
| | CTTTGATAATGGCTTGTAAGTAAGGTAGCTTAATAATGTCTGATTCTT | |
| | GAATTTGTCTATCCTTGCCCAAGACTTGCTCAATTTCTTCTTGAGCTT | |
| | TTTCCATCATCTTAGGATTTCGAATTAACTCTGCCATTACCCATTCAA | |
| | AAGTACTTGATGTAGTGTCAGTCCCAGCATCAAAAATATCCACGAGCA | |
| | GATGATTTATCTCGCCCATACTGAGCTCTTTTTGTTTGTGGAGGTCAA | |
| | GAAGAATGTCTAGCACATCATCGGTCGTCTTAGTCGAAGCAGAATCCT | |
| | GGCCGTTGCCAATCCTTTGACATATTATGCTTTGAAAAACTGCAATCA | |
| | ACTTGTCAAAATTAGACGCCAATCGACGTCGAATTCCTGATGGGTCTA | |
| | AGCATCCTAAGATTGGAAAATAATCAGCGTAGTTAGGTTTGCCAATAT | |
| | CTTCCATAATATTCCATATAAGTTCTTTGAATTGTTGAGATGTATGGG | |
| | ATTTATGGTGGGCTAATTCGACCGAAAAGAATAATTTTGATAACAAAT | |
| | TAAGGGAGGTTGTAAATGCTGCCTTGCCAATATCAACAGCTTGTCCTT | |
| | TTTGTGCACATTCTTGTACATATTGGAAAAGCTGTTGCACTTTGGCAT | |
| | GTCTTAGGGTTTGGCAAGCATCAAGTCTTTGAGGAGAAAGTAAATGAA | |
| | CGGCGGTGATTTTTCTAAAGTTCCGCCACTTTGGAGAAACAGGCAACC | |
| | ACGACATAGTCAGCTTATGGTGATCCCCAGCAGTGACAGAATTAGGAA | |
| | CAGTGCGGTTAGAAAGTGGGTGGTCTTTTTTTAAGAACATTTCTTTGG | |
| | CTACTTCAGCCGAGGATACAACAATAGTTGTGACACTTCCTAGACGTA | |
| | ATGATATCAATGGACCATGAATCTTAGCAAGATTAGCAAATGACTGGT | |
| | GAGGTTTCTCACCAACTTCAAGGATGTTTCCGATTAGTGGCAATGGTT | |
| | TAGGACCGGGGGGAAGAAGTTTGGTAGTTTGGCTAGTGAATAACATTT | |
| | TAATGAAATGAAAAACAACAAACCAAATAGCAAGTATCATTGCTAGTG | |
| | TTGCATGATCCATTCTGCAAAAGAGAACCAGACAACAGGGTAAGTGCC | |
| | TAGCAGTAAACAAACAGAACTCATCACAAGCAAACAGCAACATCATAT | |
| | TCATACCAACAGGTCATGTGTGTTCATCACATCATTAGTACTAAGCAT | |
| | GCCATCATCCAAGTATATCAAAGTAAGGGCAAAGAGCATGCATGATCA | |
| | TCAGGTGCACAAAAGAATCATCAAATTGTAGCAGTACATATCTTCATC | |
| | TATCATGCATATCTATCCATAACAGGACGATGCATGTTGACCAGGTAA | |
| | AAGCTACAGGATCCTATAGGAACAGCAGCGTATATATCTTCACCAATC | |
| | TTAGCATCTTAATCATGTGGCACATGCAGTTTCAATTTAAGCACATGA | |
| | GCTAGTTGATTATGAGGTACCAGAGAATCATCAAACTGATGTAGCAGC | |
| | ATATATCCTCATCTATCATGCAATCTAATCTAATCTAACTAAACAGGA | |
| | AAGGTGTGCTATTCAGTTAAAAGCTACCGCATCATATACAAACGGCAG | |
| | CATAAGAAAAAGCATAATCATCTTAATCATGCAACAAACGCAGATTCA | |
| | TAATAAGCCCAAGAGCTAGCTTGTGATGATCTTATTCTACTCTGATCT | |
| | ACAGCAATCAGATAACGACCTAACCTTGCACATGGCAACAAAACAATC | |
| | GATCGGACGAATCAGTTGTTTGTTCCTAGCTAGCACCATCGAACCAGA | |
| | TAATAGATGCACGTACAGATCCCGAAAACGAACCCAAAAACAGGGCAG | |
| | ACCTAGCTGAACCTAGGCAGCGACCCAGCAGATCGTGAGAACGATCTC | |
| | ATCTACGAACAGCCTAGAAGCAACCCCACGATTCCCGGACAAACGACC | |
| | TAAAATCCCCACAAATCACATGAGCATGACAGGATAAACAGCGGAACC | |
| | GATCAGATCTACACGAAAACCCCACCTCCCAGCCACCCACGATCAGGA | |
| | AACACGCGGATCTAGCATGATTTCGTCAACGCCTCAGCCTAGTTCCTA | |
| | GCCACAGACCAAGCAGAACCACCAAACCACGCCGAGCGAGGAGATGGG | |
| | GCAAGAGGACGGGGGAGACGATCGCCGTACCTTGAAGCGGGGGAAGGA | |
| | TCGCCGAGGGTCGCGAGAAGAGCAATTTGGATTTGGAACCCGGGGGTT | |
| | GTGCGCTCCGAACGATGAGACGATGTGAGATTGTGGGAAGAGGCGCGG | |
| | AGGGCCCTGTATTTATGGGCTGCGACGGGGGGAGGAGAGGTGGGGAGG | |
| | GTTGGGGAAGGAATCCCCCACCCGTGCCGTGACGGTTCCGGGCCGTGT | |
| | GAGAGGAGCCCGCTCGTCTCCGCCACGCAATTTCCGCGATCGGAGCGG | |
| | AGCTTTCGAGAGGCGGCTGGATGGTTGGTGGCCGTTAGATTTGTAGAC | |
| | GCCGTTAACGCCTCGCCTCCACCGGGAAGAGTTTTGAGCAGCCGCTTA | |
| | TGACAATGGCTTAACGACGTTAGACGGAGCGTTAGTGGCAGGCCATGA | |
| | TAGAATAGACGTATTGCAATGGGATTATAATTAAATAAATAAGAATAT | |
| | AATAAGATATGGCAAGTCGGCACTCATGACATGGTCTGCGAAATGATA | |
| | GTGCTCACTTTCTTAGCCGAGAAAGTTGACGCGACTGATTTAGAAGTT | |
| | AAGATATTATTTCTCTCTTCTTTTCTTTCCTCGTCATATAAGGATGAA | |
| | ATAAACTTTAGAGATTGCCGGTAGTGATTTTGGATTTCGGCGATCAGG | |
| | CTTGGTTTGCCGGTTTCGGACGGTGTGCCTTAGGCCACCCGCAGTGTA | |
| | TCTTGTAATGTTCAACCGATAAGCAAGGGTGGGGCTCAAGCAAGTAGT | |
| | AAACAACTATGTCAAATGTCACCATGGTTATGGTCTTGTTTAGTTGGC | |
| | TTCCTCCAAGCTTGAATTCTTGCAGCGATCTGGATTTTAGTACTGGAT | |
| | TTTGGTTTTAGGAATTAGAAATTTTATTGATAGAAGTATTTTACAAAT | |
| | ACAAATACATACTAAGGGTTTCTTATATGCTCAACACATGAGCGAAAC | |
| | CCTATAGGAACCCTAATTCCCTTATCTGGGAACTACTCACACATTATT | |
| | ATGGAGAAACTCGAGCTTGTCGATCGACTCTAGCTAGAGAAGCTTACT | |
| | TGTACAGCTCGTCCATGCCGAGAGTGATCCCGGCGGCGGTCACGAACT | |
| | CCAGCAGGACCATGTGATCGCGCTTCTCGTTGGGGTCTTTGCTCAGGG | |
| | CGGACTGGGTGCTCAGGTAGTGGTTGTCGGGCAGCAGCACGGGGCCGT | |
| | CGCCGATGGGGGTGTTCTGCTGGTAGTGGTCGGCGAGCTGCACGCTGC | |
| | CGTCCTCGATGTTGTGGCGGATCTTGAAGTTCACCTTGATGCCGTTCT | |
| | TCTGCTTGTCGGCCATGATATAGACGTTGTGGCTGTTGTAGTTGTACT | |
| | CCAGCTTGTGCCCCAGGATGTTGCCGTCCTCCTTGAAGTCGATGCCCT | |
| | TCAGCTCGATGCGGTTCACCAGGGTGTCGCCCTCGAACTTCACCTCGG | |
| | CGCGGGTCTTGTAGTTGCCGTCGTCCTTGAAGAAGATGGTGCGCTCCT | |
| | GGACGTAGCCTTCGGGCATGGCGGACTTGAAGAAGTCGTGCTGCTTCA | |
| | TGTGGTCGGGGTAGCGGCTGAAGCACTGCACGCCGTAGGTCAGGGTGG | |
| | TCACGAGGGTGGGCCAGGGCACGGGCAGCTTGCCGGTGGTGCAGATGA | |
| | ACTTCAGGGTCAGCTTGCCGTAGGTGGCATCGCCCTCGCCCTCGCCGG | |
| | ACACGCTGAACTTGTGGCCGTTTACGTCGCCGTCCAGCTCGACCAGGA | |
| | TGGGCACCACCCCGGTGAACAGCTCCTCGCCCTTGCTCACCATTCTGC | |
| | AAAAGAGAACCAGACAACAGGGTAAGTGCCTAGCAGTAAACAAACAGA | |
| | ACTCATCACAAGCAAACAGCAACATCATATTCATACCAACAGGTCATG | |
| | TGTGTTCATCACATCATTAGTACTAAGCATGCCATCATCCAAGTATAT | |
| | CAAAGTAAGGGCAAAGAGCATGCATGATCATCAGGTGCACAAAAGAAT | |
| | CATCAAATTGTAGCAGTACATATCTTCATCTATCATGCATATCTATCC | |
| | ATAACAGGACGATGCATGTTGACCAGGTAAAAGCTACAGGATCCTATA | |
| | GGAACAGCAGCGTATATATCTTCACCAATCTTAGCATCTTAATCATGT | |
| | GGCACATGCAGTTTCAATTTAAGCACATGAGCTAGTTGATTATGAGGT | |
| | ACCAGAGAATCATCAAACTGATGTAGCAGCATATATCCTCATCTATCA | |
| | TGCAATCTAATCTAATCTAACTAAACAGGAAAGGTGTGCTATTCAGTT | |
| | AAAAGCTACCGCATCATATACAAACGGCAGCATAAGAAAAAGCATAAT | |
| | CATCTTAATCATGCAACAAACGCAGATTCATAATAAGCCCAAGAGCTA | |
| | GCTTGTGATGATCTTATTCTACTCTGATCTACAGCAATCAGATAACGA | |
| | CCTAACCTTGCACATGGCAACAAAACAATCGATCGGACGAATCAGTTG | |
| | TTTGTTCCTAGCTAGCACCATCGAACCAGATAATAGATGCACGTACAG | |
| | ATCCCGAAAACGAACCCAAAAACAGGGCAGACCTAGCTGAACCTAGGC | |
| | AGCGACCCAGCAGATCGTGAGAACGATCTCATCTACGAACAGCCTAGA | |
| | AGCAACCCCACGATTCCCGGACAAACGACCTAAAATCCCCACAAATCA | |
| | CATGAGCATGACAGGATAAACAGCGGAACCGATCAGATCTACACGAAA | |
| | ACCCCACCTCCCAGCCACCCACGATCAGGAAACACGCGGATCTAGCAT | |
| | GATTTCGTCAACGCCTCAGCCTAGTTCCTAGCCACAGACCAAGCAGAA | |
| | CCACCAAACCACGCCGAGCGAGGAGATGGGGCAAGAGGACGGGGGAGA | |
| | CGATCGCCGTACCTTGAAGCGGGGGAAGGATCGCCGAGGGTCGCGAGA | |
| | AGAGCAATTTGGATTTGGAACCCGGGGGTTGTGCGCTCCGAACGATGA | |
| | GACGATGTGAGATTGTGGGAAGAGGCGCGGAGGGCCCTGTATTTATGG | |
| | GCTGCGACGGGGGGAGGAGAGGTGGGGAGGGTTGGGGAAGGAATCCCC | |
| | CACCCGTGCCGTGACGGTTCCGGGCCGTGTGAGAGGAGCCCGCTCGTC | |
| | TCCGCCACGCAATTTCCGCGATCGGAGCGGAGCTTTCGAGAGGCGGCT | |
| | GGATGGTTGGTGGCCGTTAGATTTGTAGACGCCGTTAACGCCTCGCCT | |
| | CCACCGGGAAGAGTTTTGAGCAGCCGCTTATGACAATGGCTTAACGAC | |
| | GTTAGACGGAGCGTTAGTGGCAGGCCATGATAGAATAGACGTATTGCA | |
| | ATGGGATTATAATTAAATAAATAAGAATATAATAAGATATGGCAAGTC | |
| | GGCACTCATGACATGGTCTGCGAAATGATAGTGCTCACTTTCTTAGCC | |
| | GAGAAAGTTGACGCGACTGATTTAGAAGTTAAGATATTATTTCTCTCT | |
| | TCTTTTCTTTCCTCGTCATATAAGGATGAAATAAACTTTAGAGATTGC | |
| | CGGTAGTGATTTTGGATTTCGGCGATCAGGCTTGGTTTGCCGGTTTCG | |
| | GACGGTGTGCCTTAGGCCACCCGCAGTGTATCTTGTAATGTTCAACCG | |
| | ATAAGCAAGGGTGGGGCTCAAGCAAGTAGTAAACAACTATGTCAAATG | |
| | TCACCATGGTTATGGTCTTGTTTAGTTGGCTTCCTCCGGATCCGAATT | |
| | CGGCATGAGACCCACAGAGTGTTCAACCCCAGTACATTAAAAACGTCC | |
| | GCAATGTGTTATTAAGTTGTCTAAGCGTCAATTTGTTTACACCACAAT | |
| | ATATCCTGCCACCAGCCAGCCAACAGCTCCCCGACCGGCAGCTCGGCA | |
| | CAAAATCACCACTCGATACAGGCAGCCCATCAACCATGACTACTGTCC | |
| | GGTCCAACAGATCCGAGCAGGGGCTGCTGAACCCCCAGCCGGAACTGA | |
| | CCCCACAAGGCCCTAGCGTTTGCAATGCACCAGGTCATCATTGACCCA | |
| | GGCGTGTTCCACCAGGCCGCTGCCTCGCAACTCTTCGCAGGCTTCGCC | |
| | GACCTGCTCGCGCCACTTCTTCACGCGGGTGGAATCCGATCCGCACAT | |
| | GAGGCGGAAGGTTTCCAGCTTGAGCGGGTACGGCTCCCGGTGCGAGCT | |
| | GAAATAGTCGAACATCCGTCGGGCCGTCGGCGACAGCTTGCGGTACTT | |
| | CTCCCATATGAATTTCGTGTAGTGGTCGCCAGCAAACAGCACGACGAT | |
| | TTCCTCGTCGATCAGGACCTGGCAACGGGACGTTTTCTTGCCACGGTC | |
| | CAGGACGCGGAAGCGGTGCAGCAGCGACACCGATTCCAGGTGCCCAAC | |
| | GCGGTCGGACGTGAAGCCCATCGCCGTCGCCTGTAGGCGCGACAGGCA | |
| | TTCCTCGGCCTTCGTGTAATACCGGCCATTGATCGACCAGCCCAGGTC | |
| | CTGGCAAAGCTCGTAGAACGTGAAGGTGATCGGCTCGCCGATAGGGGT | |
| | GCGCTTCGCGTACTCCAACACCTGCTGCCACACCAGTTCGTCATCGTC | |
| | GGCCCGCAGCTCGACGCCGGTGTAGGTGATCTTCACGTCCTTGTTGAC | |
| | GTGGAAAATGACCTTGTTTTGCAGCGCCTCGCGCGGGATTTTCTTGTT | |
| | GCGCGTGGTGAACAGGGCAGAGCGGGCCGTGTCGTTTGGCATCGCTCG | |
| | CATCGTGTCCGGCCACGGCGCAATATCGAACAAGGAAAGCTGCATTTC | |
| | CTTGATCTGCTGCTTCGTGTGTTTCAGCAACGCGGCCTGCTTGGCCTC | |
| | GCTGACCTGTTTTGCCAGGTCCTCGCCGGCGGTTTTTCGCTTCTTGGT | |
| | CGTCATAGTTCCTCGCGTGTCGATGGTCATCGACTTCGCCAAACCTGC | |
| | CGCCTCCTGTTCAAGACGACGCGAACGCTCCACGGCGGCCGATGGCGC | |
| | GGGCAGGGCAGGGGGAGCCAGTTGCACGCTGTCGCGCTCGATCTTGGC | |
| | CGTAGCTTGCTGGACCATCGAGCCGACGGACTGGAAGGTTTCGCGGGG | |
| | CGCACGCATGACGGTGCGGCTTGCGATGGTTTCGGCATCCTCGGCGGA | |
| | AAACCCCGCGTCGATCAGTTCTTGCCTGTATGCCTTCCGGTCAAACGT | |
| | CCGATTCATTCACCCTCCTTGCGGGATTGCCCCGACTCACGCCGGGGC | |
| | AATGTGCCCTTATTCCTGATTTGACCCGCCTGGTGCCTTGGTGTCCAG | |
| | ATAATCCACCTTATCGGCAATGAAGTCGGTCCCGTAGACCGTCTGGCC | |
| | GTCCTTCTCGTACTTGGTATTCCGAATCTTGCCCTGCACGAATACCAG | |
| | CGACCCCTTGCCCAAATACTTGCCGTGGGCCTCGGCCTGAGAGCCAAA | |
| | ACACTTGATGCGGAAGAAGTCGGTGCGCTCCTGCTTGTCGCCGGCATC | |
| | GTTGCGCCACATCTAGGATCTGCCAGGAACCGTAAAAAGGCCGCGTTG | |
| | CTGGCGTTTTTCCATAGGCTCCGCCCCCCTGACGAGCATCACAAAAAT | |
| | CGACGCTCAAGTCAGAGGTGGCGAAACCCGACAGGACTATAAAGATAC | |
| | CAGGCGTTTCCCCCTGGAAGCTCCCTCGTGCGCTCTCCTGTTCCGACC | |
| | CTGCCGCTTACCGGATACCTGTCCGCCTTTCTCCCTTCGGGAAGCGTG | |
| | GCGCTTTCTCATAGCTCACGCTGTAGGTATCTCAGTTCGGTGTAGGTC | |
| | GTTCGCTCCAAGCTGGGCTGTGTGCACGAACCCCCCGTTCAGCCCGAC | |
| | CGCTGCGCCTTATCCGGTAACTATCGTCTTGAGTCCAACCCGGTAAGA | |
| | CACGACTTATCGCCACTGGCAGCAGCCACTGGTAACAGGATTAGCAGA | |
| | GCGAGGTATGTAGGCGGTGCTACAGAGTTCTTGAAGTGGTGGCCTAAC | |
| | TACGGCTACACTAGAAGGACAGTATTTGGTATCTGCGCTCTGCTGAAG | |
| | CCAGTTACCTTCGGAAAAAGAGTTGGTAGCTCTTGATCCGGCAAACAA | |
| | ACCACCGCTGGTAGCGGTGGTTTTTTTGTTTGCAAGCAGCAGATTACG | |
| | CGCAGAAAAAAAGGATCTCAAGAAGATCCTTTGATCTTTTCTACGGGG | |
| | TCTGACGCTCAGTGGAACGAAAACTCACGTTAAGGGATTTTGGTCATG | |
| | AGATTATCAAAAAGGATCTTCACCTAGATCCTTTTAAATTAAAAATGA | |
| | AGTTTTAAATCAATCTAAAGTATATATGAGTAAACTTGGTCTGACAGC | |
| | ATGATATATCTCCCAATTTGTGTAGGGCTTATTATGCACGCTTAAAAA | |
| | TAATAAAAGCAGACTTGACCTGATAGTTTGGCTGTGAGCAATTATGTG | |
| | CTTAGTGCATCTAACGCTTGAGTTAAGCCGCGCCGCGAAGCGGCGTCG | |
| | GCTTGAACGAATTTCTAGCTAGACATTATTTGCCGACTACCTTGGTGA | |
| | TCTCGCCTTTCACGTAATGGACAAATTCTTCCAACTGATCTGCGCGCG | |
| | AGGCCAAGCGATCTTCTTCTTGTCCAAGATAAGCCTGTCTAGCTTCAA | |
| | GTATGACGGGCTGATACTGGGCCGGCAGGCGCTCCATTGCCCAGTCGG | |
| | CAGCGACATCCTTCGGCGCGATTTTGCCGGTTACTGCGCTGTACCAAA | |
| | TGCGGGACAACGTAAGCACTACATTTCGCTCATCGCCAGCCCAGTCGG | |
| | GCGGCGAGTTCCATAGCGTTAAGGTTTCATTTAGCGCCTCAAATAGAT | |
| | CCTGTTCAGGAACCGGATCAAAGAGTTCCTCCGCCGCTGGACCTACCA | |
| | AGGCAACGCTATGTTCTCTTGCTTTTGTCAGCAAGATAGCCAGATCAA | |
| | TGTCGATCGTGGCTGGCTCGAAGATACCTGCAAGAATGTCATTGCGCT | |
| | GCCATTCTCCAAATTGCAGTTCGCGCTTAGCTGGATAACGCCACGGAA | |
| | TGATGTCGTCGTGCACAACAATGGTGACTTCTACAGCGCGGAGAATCT | |
| | CGCTCTCTCCAGGGGAAGCCGAAGTTTCCAAAAGGTCGTTGATCAAAG | |
| | CTCGCCGCGTTGTTTCATCAAGCCTTACGGTCACCGTAACCAGCAAAT | |
| | CAATATCGCTGTGTGGCTTCAGGCCGCCATCCACTGCGGAGCCGTACA | |
| | AATGTACGGCCAGCAACGTCGGTTCGAGATGGCGCTCGATGACGCCAA | |
| | CTACCTCTGATAGTTGAGTCGATACTTCGGCGATCACCGCTTCCCCCA | |
| | TGATGTTTAACTTTGTTTTAGGGCGACTGCCCTGCTGCGTAACATCGT | |
| | TGCTGCTCCATAACATCAAACATCGACCCACGGCGTAACGCGCTTGCT | |
| | GCTTGGATGCCCGAGGCATAGACTGTACCCCAAAAAAACAGTCATAAC | |
| | AAGCCATGAAAACCGCCACTGCGCCGTTACCACCGCTGCGTTCGGTCA | |
| | AGGTTCTGGACCAGTTGCGTGAGCGCATACGCTACTTGCATTACAGCT | |
| | TACGAACCGAACAGGCTTATGTCCACTGGGTTCGTGCCTCGGGAATTG | |
| | GCCAGCGCTGCCATTTTTGGGGTGAGGCCGTTCGCGGCCGAGGGGCGC | |
| | AGCCCCTGGGGGGATGGGAGGCCCGCGTTAGCGGGCCGGGAGGGTTCG | |
| | AGAAGGGGGGGCACCCCCCTTCGGCGTGCGCGGTCACGCGCACAGGGC | |
| | GCAGCCCTGGTTAAAAACAAGGTTTATAAATATTGGTTTAAAAGCAGG | |
| | TTAAAAGACAGGTTAGCGGTGGCCGAAAAACGGGCGGAAACCCTTGCA | |
| | AATGCTGGATTTTCTGCCTGTGGACAGCCCCTCAAATGTCAATAGGTG | |
| | CGCCCCTCATCTGTCAGCACTCTGCCCCTCAAGTGTCAAGGATCGCGC | |
| | CCCTCATCTGTCAGTAGTCGCGCCCCTCAAGTGTCAATACCGCAGGGC | |
| | ACTTATCCCCAGGCTTGTCCACATCATCTGTGGGAAACTCGCGTAAAA | |
| | TCAGGCGTTTTCGCCGATTTGCGAGGCTGGCCAGCTCCACGTCGCCGG | |
| | CCGAAATCGAGCCTGCCCCTCATCTGTCAACGCCGCGCCGGGTGAGTC | |
| | GGCCCCTCAAGTGTCAACGTCCGCCCCTCATCTGTCAGTGAGGGCCAA | |
| | GTTTTCCGCGAGGTATCCACAACGCCGGCGGCCGCGGTGTCTCGCACA | |
| | CGGCTTCGACGGCGTTTCTGGCGCGTTTGCAGGGCCATAGACGGCCGC | |
| | CAGCCCAGCGGCGAGGGCAACCAGCCCGGTGAGCGTCGCAAAGGCGGC | |
| | TGGCACATACAAATGGACGAACGGATAAACCTTTTCACGCCCTTTTAA | |
| | ATATCCGATTATTCTAATAAACGCTCTTTTCTCTTAGGTTTACCCGCC | |
| | AATATATCCTGTCAAACACTGATAGTTTAAACGGATCAC | |
| PL-464 | TGAACACTCTGTGGGTCTCATGCCGAATTCGGATCCGGAGGAAGCCAA | 198 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATCATGCAACACTAGCAATGATACTTGCTATT | |
| | TGGTTTGTTGTTTTTCATTTCATTAAAATGTTATTCACTAGCCAAACT | |
| | ACCAAACTTCTTCCCCCCGGTCCTAAACCATTGCCACTAATCGGAAAC | |
| | ATCCTTGAAGTTGGTGAGAAACCTCACCAGTCATTTGCTAATCTTGCT | |
| | AAGATTCATGGTCCATTGATATCATTACGTCTAGGAAGTGTCACAACT | |
| | ATTGTTGTATCCTCGGCTGAAGTAGCCAAAGAAATGTTCTTAAAAAAA | |
| | GACCACCCACTTTCTAACCGCACTGTTCCTAATTCTGTCACTGCTGGG | |
| | GATCACCATAAGCTGACTATGTCGTGGTTGCCTGTTTCTCCAAAGTGG | |
| | CGGAACTTTAGAAAAATCACCGCCGTTCATTTACTTTCTCCTCAAAGA | |
| | CTTGATGCTTGCCAAACCCTAAGACATGCCAAAGTGCAACAGCTTTTC | |
| | CAATATGTACAAGAATGTGCACAAAAAGGACAAGCTGTTGATATTGGC | |
| | AAGGCAGCATTTACAACCTCCCTTAATTTGTTATCAAAATTATTCTTT | |
| | TCGGTCGAATTAGCCCACCATAAATCCCATACATCTCAACAATTCAAA | |
| | GAACTTATATGGAATATTATGGAAGATATTGGCAAACCTAACTACGCT | |
| | GATTATTTTCCAATCTTAGGATGCTTAGACCCATCAGGAATTCGACGT | |
| | CGATTGGCGTCTAATTTTGACAAGTTGATTGCAGTTTTTCAAAGCATA | |
| | ATATGTCAAAGGATTGGCAACGGCCAGGATTCTGCTTCGACTAAGACG | |
| | ACCGATGATGTGCTAGACATTCTTCTTGACCTCCACAAACAAAAAGAG | |
| | CTCAGTATGGGCGAGATAAATCATCTGCTCGTGGATATTTTTGATGCT | |
| | GGGACTGACACTACATCAAGTACTTTTGAATGGGTAATGGCAGAGTTA | |
| | ATTCGAAATCCTAAGATGATGGAAAAAGCTCAAGAAGAAATTGAGCAA | |
| | GTCTTGGGCAAGGATAGACAAATTCAAGAATCAGACATTATTAAGCTA | |
| | CCTTACTTACAAGCCATTATCAAAGAAACATTGCGGCTACACCCACCA | |
| | ACTGTATTTCTCTTGCCTCGTAAAGCTGATAGTGATGTTGAATTATAT | |
| | GGCTATGTTGTGCCGAAAGATGCACAAATACTTGTTAATTTATGGGCC | |
| | ATTGGAAGGGACCCTCAAGCGTGGGTGAAACCTGACGTGTTTTTACCT | |
| | GAGAGGTTTTTAGGATCCGAAATTGATGTGAAAGGAAGAGATTTTGGA | |
| | CTCTTACCTTTTGGAGCTGGAAGGAGAATATGCCCCGGGATGAATTTG | |
| | GCTATTAGAATGTTAACTTTGATGTTAGCTACGCTTCTTCAATTCTTC | |
| | AATTGGAAGCTTGAAGAAGGTATGAAAGCAGAAGATCTAGACATGGAT | |
| | GAGAAATTTGGGATTGCCTTACAAAAGACTAAACCTCTTCAGATTATT | |
| | CCCGTTCTTAGGTATTGAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGGATTCATCAAAA | |
| | ACAAAGCACATTATCCTCTTGCCATTTCTAGCGCAAGGTCATCTCCGT | |
| | CCATTTCTGCACTTAGCCAATTTCCTCAGATCCCACACTCCTTTCACC | |
| | CTTTCTATCCTTACTACTCCTCTCAATGCCGCCAATCTCCGCCGTCAA | |
| | TCCAACAACATTAACATCTACGATCTACCCTTTAACAGCACCGATCAT | |
| | AATCTACCACCAAACACAGAAAACACTGAAAAACTCCCATTAACTTCA | |
| | ATAATCTCTCTGTTTTATGCATCCACTTCGCTCCAGCCTCACGTACGC | |
| | AACCACCTTACTCGCCATCACCTTGATAACCCACCCATTTGTATCATC | |
| | TTTGATATTTTCTTGGGCTGGGCTGATAACTTGGCCCGATCTATTGGA | |
| | TCTACCGGAGTTTGTTTCAACACTGGTGGGGCCTACGGTTTGGCCGCT | |
| | TACATGTCGATTTGGATGCATTTGCCTCATAGAAATGTTCCAGACAAT | |
| | GTCGAGTTTTCCCTTCCGGAATTCCCTGAGAATCGGAAATTTAAACGT | |
| | AACCAACTTCACAGGTTTTTAAGATTTGCTGATGGAACCGATGATTGG | |
| | TCGGGTTTTTTTCAACCCCAGATAAAGTACTCCAGGAATTGTTCTGCG | |
| | TGGTTGTGTAATTCAATTGAGGAAATTGAGCCATTAGGGTTTGAGGTT | |
| | TTAAGAGCAATGTTGAAAGTACCAGTTTGGGGAATTGGTCCTTTAGTC | |
| | AAGACTGAAAATAGTTGTGATGAAGATGATGAACAAGGTTGTGTTGAG | |
| | TGGTTAAACCAATTTGAGAAAGGATCTGTTTTGTACATATCATTTGGG | |
| | TCACAGAATACAGTTACCCCGATCCAAATGATGGAATTAGCAAAAGGA | |
| | TTGGAGGAAAGTGGGGCGAAATTTTTGTGGGTGATTCGTCCCCCTTTC | |
| | GGGTTTGATATCAATGGAGAGTTCAAACCCGAATGGCTGCCGGAGGGA | |
| | TTTGAGAAGAGAGTAATGGAAAGGAAGCAGGGGAAGCTAGTAAAGAAA | |
| | TGGGGGCCTCAAATGGAGATATTGAGGAATAAGGCGACGGGGGCGTTT | |
| | TTGAGTCACTGTGGGTGGAATTCGTTGATCGAAGCGTTAAGCGAAGGT | |
| | GTTCCGATAATAGGGTGGCCATTGGCTGCGGAGCAAGCGTATAATTCA | |
| | AAGATGTTAGTGGAAGAGATGGGGGTTGCGGTCGAGTTAACGAGGGGA | |
| | TTGGAAGGGGAGTTGAGTAGCGAAGGGGTGAAGAAGGTGGTGGAGATG | |
| | GTTATGGATAGGGAAGAAGGGAGTTTTGGGTGTGAGATGAAGAAGGCG | |
| | GCAGTTGTAATTGGACAGAAATTGAGGGATGCAATGAAAGTGGAAGGT | |
| | GATTATAGAGGGTGTTCTCTTAGATCTCTTGATGAGTTTGTAGAGTTC | |
| | ATTGTTTCTCGTAAGGCTACTTTGAGCTAGGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTCAGATGAGACCGAGGATGCACATGTGACCGAGGGACACGAA | |
| PL-465 | TGAACACTCTGTGGGTCTCATGCCGAATTCGGATCCGGAGGAAGCCAA | 199 |
| construct | CTAAACAAGACCATAACCATGGTGACATTTGACATAGTTGTTTACTAC | |
| | TTGCTTGAGCCCCACCCTTGCTTATCGGTTGAACATTACAAGATACAC | |
| | TGCGGGTGGCCTAAGGCACACCGTCCGAAACCGGCAAACCAAGCCTGA | |
| | TCGCCGAAATCCAAAATCACTACCGGCAATCTCTAAAGTTTATTTCAT | |
| | CCTTATATGACGAGGAAAGAAAAGAAGAGAGAAATAATATCTTAACTT | |
| | CTAAATCAGTCGCGTCAACTTTCTCGGCTAAGAAAGTGAGCACTATCA | |
| | TTTCGCAGACCATGTCATGAGTGCCGACTTGCCATATCTTATTATATT | |
| | CTTATTTATTTAATTATAATCCCATTGCAATACGTCTATTCTATCATG | |
| | GCCTGCCACTAACGCTCCGTCTAACGTCGTTAAGCCATTGTCATAAGC | |
| | GGCTGCTCAAAACTCTTCCCGGTGGAGGCGAGGCGTTAACGGCGTCTA | |
| | CAAATCTAACGGCCACCAACCATCCAGCCGCCTCTCGAAAGCTCCGCT | |
| | CCGATCGCGGAAATTGCGTGGCGGAGACGAGCGGGCTCCTCTCACACG | |
| | GCCCGGAACCGTCACGGCACGGGTGGGGGATTCCTTCCCCAACCCTCC | |
| | CCACCTCTCCTCCCCCCGTCGCAGCCCATAAATACAGGGCCCTCCGCG | |
| | CCTCTTCCCACAATCTCACATCGTCTCATCGTTCGGAGCGCACAACCC | |
| | CCGGGTTCCAAATCCAAATTGCTCTTCTCGCGACCCTCGGCGATCCTT | |
| | CCCCCGCTTCAAGGTACGGCGATCGTCTCCCCCGTCCTCTTGCCCCAT | |
| | CTCCTCGCTCGGCGTGGTTTGGTGGTTCTGCTTGGTCTGTGGCTAGGA | |
| | ACTAGGCTGAGGCGTTGACGAAATCATGCTAGATCCGCGTGTTTCCTG | |
| | ATCGTGGGTGGCTGGGAGGTGGGGTTTTCGTGTAGATCTGATCGGTTC | |
| | CGCTGTTTATCCTGTCATGCTCATGTGATTTGTGGGGATTTTAGGTCG | |
| | TTTGTCCGGGAATCGTGGGGTTGCTTCTAGGCTGTTCGTAGATGAGAT | |
| | CGTTCTCACGATCTGCTGGGTCGCTGCCTAGGTTCAGCTAGGTCTGCC | |
| | CTGTTTTTGGGTTCGTTTTCGGGATCTGTACGTGCATCTATTATCTGG | |
| | TTCGATGGTGCTAGCTAGGAACAAACAACTGATTCGTCCGATCGATTG | |
| | TTTTGTTGCCATGTGCAAGGTTAGGTCGTTATCTGATTGCTGTAGATC | |
| | AGAGTAGAATAAGATCATCACAAGCTAGCTCTTGGGCTTATTATGAAT | |
| | CTGCGTTTGTTGCATGATTAAGATGATTATGCTTTTTCTTATGCTGCC | |
| | GTTTGTATATGATGCGGTAGCTTTTAACTGAATAGCACACCTTTCCTG | |
| | TTTAGTTAGATTAGATTAGATTGCATGATAGATGAGGATATATGCTGC | |
| | TACATCAGTTTGATGATTCTCTGGTACCTCATAATCAACTAGCTCATG | |
| | TGCTTAAATTGAAACTGCATGTGCCACATGATTAAGATGCTAAGATTG | |
| | GTGAAGATATATACGCTGCTGTTCCTATAGGATCCTGTAGCTTTTACC | |
| | TGGTCAACATGCATCGTCCTGTTATGGATAGATATGCATGATAGATGA | |
| | AGATATGTACTGCTACAATTTGATGATTCTTTTGTGCACCTGATGATC | |
| | ATGCATGCTCTTTGCCCTTACTTTGATATACTTGGATGATGGCATGCT | |
| | TAGTACTAATGATGTGATGAACACACATGACCTGTTGGTATGAATATG | |
| | ATGTTGCTGTTTGCTTGTGATGAGTTCTGTTTGTTTACTGCTAGGCAC | |
| | TTACCCTGTTGTCTGGTTCTCTTTTGCAGAATGGTGAGCAAGGGCGAG | |
| | GAGCTGTTCACCGGGGTGGTGCCCATCCTGGTCGAGCTGGACGGCGAC | |
| | GTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTG | |
| | CCCGTGCCCTGGCCCACCCTCGTGACCACCCTGACCTACGGCGTGCAG | |
| | TGCTTCAGCCGCTACCCCGACCACATGAAGCAGCACGACTTCTTCAAG | |
| | TCCGCCATGCCCGAAGGCTACGTCCAGGAGCGCACCATCTTCTTCAAG | |
| | GACGACGGCAACTACAAGACCCGCGCCGAGGTGAAGTTCGAGGGCGAC | |
| | ACCCTGGTGAACCGCATCGAGCTGAAGGGCATCGACTTCAAGGAGGAC | |
| | GGCAACATCCTGGGGCACAAGCTGGAGTACAACTACAACAGCCACAAC | |
| | GTCTATATCATGGCCGACAAGCAGAAGAACGGCATCAAGGTGAACTTC | |
| | AAGATCCGCCACAACATCGAGGACGGCAGCGTGCAGCTCGCCGACCAC | |
| | TACCAGCAGAACACCCCCATCGGCGACGGCCCCGTGCTGCTGCCCGAC | |
| | AACCACTACCTGAGCACCCAGTCCGCCCTGAGCAAAGACCCCAACGAG | |
| | AAGCGCGATCACATGGTCCTGCTGGAGTTCGTGACCGCCGCCGGGATC | |
| | ACTCTCGGCATGGACGAGCTGTACAAGTAAGCTTCTCTAGCTAGAGTC | |
| | GATCGACAAGCTCGAGTTTCTCCATAATAATGTGTGAGTAGTTCCCAG | |
| | ATAAGGGAATTAGGGTTCCTATAGGGTTTCGCTCATGTGTTGAGCATA | |
| | TAAGAAACCCTTAGTATGTATTTGTATTTGTAAAATACTTCTATCAAT | |
| | AAAATTTCTAATTCCTAAAACCAAAATCCAGTACTAAAATCCAGATCG | |
| | CTGCAAGAATTCAAGCTTGGAGGAAGCCAACTAAACAAGACCATAACC | |
| | ATGGTGACATTTGACATAGTTGTTTACTACTTGCTTGAGCCCCACCCT | |
| | TGCTTATCGGTTGAACATTACAAGATACACTGCGGGTGGCCTAAGGCA | |
| | CACCGTCCGAAACCGGCAAACCAAGCCTGATCGCCGAAATCCAAAATC | |
| | ACTACCGGCAATCTCTAAAGTTTATTTCATCCTTATATGACGAGGAAA | |
| | GAAAAGAAGAGAGAAATAATATCTTAACTTCTAAATCAGTCGCGTCAA | |
| | CTTTCTCGGCTAAGAAAGTGAGCACTATCATTTCGCAGACCATGTCAT | |
| | GAGTGCCGACTTGCCATATCTTATTATATTCTTATTTATTTAATTATA | |
| | ATCCCATTGCAATACGTCTATTCTATCATGGCCTGCCACTAACGCTCC | |
| | GTCTAACGTCGTTAAGCCATTGTCATAAGCGGCTGCTCAAAACTCTTC | |
| | CCGGTGGAGGCGAGGCGTTAACGGCGTCTACAAATCTAACGGCCACCA | |
| | ACCATCCAGCCGCCTCTCGAAAGCTCCGCTCCGATCGCGGAAATTGCG | |
| | TGGCGGAGACGAGCGGGCTCCTCTCACACGGCCCGGAACCGTCACGGC | |
| | ACGGGTGGGGGATTCCTTCCCCAACCCTCCCCACCTCTCCTCCCCCCG | |
| | TCGCAGCCCATAAATACAGGGCCCTCCGCGCCTCTTCCCACAATCTCA | |
| | CATCGTCTCATCGTTCGGAGCGCACAACCCCCGGGTTCCAAATCCAAA | |
| | TTGCTCTTCTCGCGACCCTCGGCGATCCTTCCCCCGCTTCAAGGTACG | |
| | GCGATCGTCTCCCCCGTCCTCTTGCCCCATCTCCTCGCTCGGCGTGGT | |
| | TTGGTGGTTCTGCTTGGTCTGTGGCTAGGAACTAGGCTGAGGCGTTGA | |
| | CGAAATCATGCTAGATCCGCGTGTTTCCTGATCGTGGGTGGCTGGGAG | |
| | GTGGGGTTTTCGTGTAGATCTGATCGGTTCCGCTGTTTATCCTGTCAT | |
| | GCTCATGTGATTTGTGGGGATTTTAGGTCGTTTGTCCGGGAATCGTGG | |
| | GGTTGCTTCTAGGCTGTTCGTAGATGAGATCGTTCTCACGATCTGCTG | |
| | GGTCGCTGCCTAGGTTCAGCTAGGTCTGCCCTGTTTTTGGGTTCGTTT | |
| | TCGGGATCTGTACGTGCATCTATTATCTGGTTCGATGGTGCTAGCTAG | |
| | GAACAAACAACTGATTCGTCCGATCGATTGTTTTGTTGCCATGTGCAA | |
| | GGTTAGGTCGTTATCTGATTGCTGTAGATCAGAGTAGAATAAGATCAT | |
| | CACAAGCTAGCTCTTGGGCTTATTATGAATCTGCGTTTGTTGCATGAT | |
| | TAAGATGATTATGCTTTTTCTTATGCTGCCGTTTGTATATGATGCGGT | |
| | AGCTTTTAACTGAATAGCACACCTTTCCTGTTTAGTTAGATTAGATTA | |
| | GATTGCATGATAGATGAGGATATATGCTGCTACATCAGTTTGATGATT | |
| | CTCTGGTACCTCATAATCAACTAGCTCATGTGCTTAAATTGAAACTGC | |
| | ATGTGCCACATGATTAAGATGCTAAGATTGGTGAAGATATATACGCTG | |
| | CTGTTCCTATAGGATCCTGTAGCTTTTACCTGGTCAACATGCATCGTC | |
| | CTGTTATGGATAGATATGCATGATAGATGAAGATATGTACTGCTACAA | |
| | TTTGATGATTCTTTTGTGCACCTGATGATCATGCATGCTCTTTGCCCT | |
| | TACTTTGATATACTTGGATGATGGCATGCTTAGTACTAATGATGTGAT | |
| | GAACACACATGACCTGTTGGTATGAATATGATGTTGCTGTTTGCTTGT | |
| | GATGAGTTCTGTTTGTTTACTGCTAGGCACTTACCCTGTTGTCTGGTT | |
| | CTCTTTTGCAGAATGGATCATGCAACACTAGCAATGATACTTGCTATT | |
| | TGGTTTGTTGTTTTTCATTTCATTAAAATGTTATTCACTAGCCAAACT | |
| | ACCAAACTTCTTCCCCCCGGTCCTAAACCATTGCCACTAATCGGAAAC | |
| | ATCCTTGAAGTTGGTGAGAAACCTCACCAGTCATTTGCTAATCTTGCT | |
| | AAGATTCATGGTCCATTGATATCATTACGTCTAGGAAGTGTCACAACT | |
| | ATTGTTGTATCCTCGGCTGAAGTAGCCAAAGAAATGTTCTTAAAAAAA | |
| | GACCACCCACTTTCTAACCGCACTGTTCCTAATTCTGTCACTGCTGGG | |
| | GATCACCATAAGCTGACTATGTCGTGGTTGCCTGTTTCTCCAAAGTGG | |
| | CGGAACTTTAGAAAAATCACCGCCGTTCATTTACTTTCTCCTCAAAGA | |
| | CTTGATGCTTGCCAAACCCTAAGACATGCCAAAGTGCAACAGCTTTTC | |
| | CAATATGTACAAGAATGTGCACAAAAAGGACAAGCTGTTGATATTGGC | |
| | AAGGCAGCATTTACAACCTCCCTTAATTTGTTATCAAAATTATTCTTT | |
| | TCGGTCGAATTAGCCCACCATAAATCCCATACATCTCAACAATTCAAA | |
| | GAACTTATATGGAATATTATGGAAGATATTGGCAAACCTAACTACGCT | |
| | GATTATTTTCCAATCTTAGGATGCTTAGACCCATCAGGAATTCGACGT | |
| | CGATTGGCGTCTAATTTTGACAAGTTGATTGCAGTTTTTCAAAGCATA | |
| | ATATGTCAAAGGATTGGCAACGGCCAGGATTCTGCTTCGACTAAGACG | |
| | ACCGATGATGTGCTAGACATTCTTCTTGACCTCCACAAACAAAAAGAG | |
| | CTCAGTATGGGCGAGATAAATCATCTGCTCGTGGATATTTTTGATGCT | |
| | GGGACTGACACTACATCAAGTACTTTTGAATGGGTAATGGCAGAGTTA | |
| | ATTCGAAATCCTAAGATGATGGAAAAAGCTCAAGAAGAAATTGAGCAA | |
| | GTCTTGGGCAAGGATAGACAAATTCAAGAATCAGACATTATTAAGCTA | |
| | CCTTACTTACAAGCCATTATCAAAGAAACATTGCGGCTACACCCACCA | |
| | ACTGTATTTCTCTTGCCTCGTAAAGCTGATAGTGATGTTGAATTATAT | |
| | GGCTATGTTGTGCCGAAAGATGCACAAATACTTGTTAATTTATGGGCC | |
| | ATTGGAAGGGACCCTCAAGCGTGGGTGAAACCTGACGTGTTTTTACCT | |
| | GAGAGGTTTTTAGGATCCGAAATTGATGTGAAAGGAAGAGATTTTGGA | |
| | CTCTTACCTTTTGGAGCTGGAAGGAGAATATGCCCCGGGATGAATTTG | |
| | GCTATTAGAATGTTAACTTTGATGTTAGCTACGCTTCTTCAATTCTTC | |
| | AATTGGAAGCTTGAAGAAGGTATGAAAGCAGAAGATCTAGACATGGAT | |
| | GAGAAATTTGGGATTGCCTTACAAAAGACTAAACCTCTTCAGATTATT | |
| | CCCGTTCTTAGGTATTGAGCTTCTCTAGCTAGAGTCGATCGACAAGCT | |
| | CGAGTTTCTCCATAATAATGTGTGAGTAGTTCCCAGATAAGGGAATTA | |
| | GGGTTCCTATAGGGTTTCGCTCATGTGTTGAGCATATAAGAAACCCTT | |
| | AGTATGTATTTGTATTTGTAAAATACTTCTATCAATAAAATTTCTAAT | |
| | TCCTAAAACCAAAATCCAGTACTAAAATCCAGATCGCTACTAGAATTC | |
| | GAGCTCGGAGGAAGCCAACTAAACAAGACCATAACCATGGTGACATTT | |
| | GACATAGTTGTTTACTACTTGCTTGAGCCCCACCCTTGCTTATCGGTT | |
| | GAACATTACAAGATACACTGCGGGTGGCCTAAGGCACACCGTCCGAAA | |
| | CCGGCAAACCAAGCCTGATCGCCGAAATCCAAAATCACTACCGGCAAT | |
| | CTCTAAAGTTTATTTCATCCTTATATGACGAGGAAAGAAAAGAAGAGA | |
| | GAAATAATATCTTAACTTCTAAATCAGTCGCGTCAACTTTCTCGGCTA | |
| | AGAAAGTGAGCACTATCATTTCGCAGACCATGTCATGAGTGCCGACTT | |
| | GCCATATCTTATTATATTCTTATTTATTTAATTATAATCCCATTGCAA | |
| | TACGTCTATTCTATCATGGCCTGCCACTAACGCTCCGTCTAACGTCGT | |
| | TAAGCCATTGTCATAAGCGGCTGCTCAAAACTCTTCCCGGTGGAGGCG | |
| | AGGCGTTAACGGCGTCTACAAATCTAACGGCCACCAACCATCCAGCCG | |
| | CCTCTCGAAAGCTCCGCTCCGATCGCGGAAATTGCGTGGCGGAGACGA | |
| | GCGGGCTCCTCTCACACGGCCCGGAACCGTCACGGCACGGGTGGGGGA | |
| | TTCCTTCCCCAACCCTCCCCACCTCTCCTCCCCCCGTCGCAGCCCATA | |
| | AATACAGGGCCCTCCGCGCCTCTTCCCACAATCTCACATCGTCTCATC | |
| | GTTCGGAGCGCACAACCCCCGGGTTCCAAATCCAAATTGCTCTTCTCG | |
| | CGACCCTCGGCGATCCTTCCCCCGCTTCAAGGTACGGCGATCGTCTCC | |
| | CCCGTCCTCTTGCCCCATCTCCTCGCTCGGCGTGGTTTGGTGGTTCTG | |
| | CTTGGTCTGTGGCTAGGAACTAGGCTGAGGCGTTGACGAAATCATGCT | |
| | AGATCCGCGTGTTTCCTGATCGTGGGTGGCTGGGAGGTGGGGTTTTCG | |
| | TGTAGATCTGATCGGTTCCGCTGTTTATCCTGTCATGCTCATGTGATT | |
| | TGTGGGGATTTTAGGTCGTTTGTCCGGGAATCGTGGGGTTGCTTCTAG | |
| | GCTGTTCGTAGATGAGATCGTTCTCACGATCTGCTGGGTCGCTGCCTA | |
| | GGTTCAGCTAGGTCTGCCCTGTTTTTGGGTTCGTTTTCGGGATCTGTA | |
| | CGTGCATCTATTATCTGGTTCGATGGTGCTAGCTAGGAACAAACAACT | |
| | GATTCGTCCGATCGATTGTTTTGTTGCCATGTGCAAGGTTAGGTCGTT | |
| | ATCTGATTGCTGTAGATCAGAGTAGAATAAGATCATCACAAGCTAGCT | |
| | CTTGGGCTTATTATGAATCTGCGTTTGTTGCATGATTAAGATGATTAT | |
| | GCTTTTTCTTATGCTGCCGTTTGTATATGATGCGGTAGCTTTTAACTG | |
| | AATAGCACACCTTTCCTGTTTAGTTAGATTAGATTAGATTGCATGATA | |
| | GATGAGGATATATGCTGCTACATCAGTTTGATGATTCTCTGGTACCTC | |
| | ATAATCAACTAGCTCATGTGCTTAAATTGAAACTGCATGTGCCACATG | |
| | ATTAAGATGCTAAGATTGGTGAAGATATATACGCTGCTGTTCCTATAG | |
| | GATCCTGTAGCTTTTACCTGGTCAACATGCATCGTCCTGTTATGGATA | |
| | GATATGCATGATAGATGAAGATATGTACTGCTACAATTTGATGATTCT | |
| | TTTGTGCACCTGATGATCATGCATGCTCTTTGCCCTTACTTTGATATA | |
| | CTTGGATGATGGCATGCTTAGTACTAATGATGTGATGAACACACATGA | |
| | CCTGTTGGTATGAATATGATGTTGCTGTTTGCTTGTGATGAGTTCTGT | |
| | TTGTTTACTGCTAGGCACTTACCCTGTTGTCTGGTTCTCTTTTGCAGA | |
| | ATGAAAATGAGTGGAGAAGATAATAGCATGATCAAAGAAACTTTCTTC | |
| | ATAACCCATGGAAACCCGATTTTGACCGTAGAAGATACACATCCCTTA | |
| | AGGCCATTCTTTGAAACATGGAGAGAAAAAATCTTTACGATGAAGCCG | |
| | AAGGCGATACTTGTGATCTCTGGTCACTGGGAAACTGATCATCCTGCT | |
| | GTTAATGCTGTTCATATCAATGATACTATCTACGATTTCGATGACTAT | |
| | CCTCAAGCCATGTACAAGTTGAAGTATCCAGCTCCAGGATCACCAGAC | |
| | TTGGCTAGCAGAGTAGAAGAAATTATGAAAAAATCCGGGTTCGACACA | |
| | GTGCACATTGACAAAAAACGTGGGCTTGATCATGGTGCATGGGTGCCT | |
| | CTTATGTACATGTATCCGGAGGCTGACATACCGGTATGTCAGCTCTCC | |
| | GTCCAGCCGAAGTTGGACGGAACGTACCACTACAACTTGGGACGAGCA | |
| | TTAGCGCCTCTAAGAGACGAAGGTGTACTCATTATTGGTTCCGGAAGT | |
| | GCAACACACCCTTTGGACGAAACCCCTCATTATCATGGTGGTGTTGCT | |
| | CCTTGGGCTGCTGACTTTGACTCATGGCTTGACGTAGCTCTCACTAAA | |
| | GGAAGATTTGAAGAAGTGAATACATATGAAACCAAAGCACCAAACTGG | |
| | GAATTGGCACATCCATTCCCGGAGCATTTTTATCCATTGCATGTCGTG | |
| | GTTGGGGCGGCTGGTGAAAAGTGGAAGGCTGAGCTTATTCATACTAGT | |
| | TGGGACCATGGTACCTTATGTCATGGCTCCTACAAGTTTACTTCCACC | |
| | TAAGCTTCTCTAGCTAGAGTCGATCGACAAGCTCGAGTTTCTCCATAA | |
| | TAATGTGTGAGTAGTTCCCAGATAAGGGAATTAGGGTTCCTATAGGGT | |
| | TTCGCTCATGTGTTGAGCATATAAGAAACCCTTAGTATGTATTTGTAT | |
| | TTGTAAAATACTTCTATCAATAAAATTTCTAATTCCTAAAACCAAAAT | |
| | CCAGTACTAAAATCCAGATCGCTTTACGAATTCCCATGGGGAGGAAGC | |
| | CAACTAAACAAGACCATAACCATGGTGACATTTGACATAGTTGTTTAC | |
| | TACTTGCTTGAGCCCCACCCTTGCTTATCGGTTGAACATTACAAGATA | |
| | CACTGCGGGTGGCCTAAGGCACACCGTCCGAAACCGGCAAACCAAGCC | |
| | TGATCGCCGAAATCCAAAATCACTACCGGCAATCTCTAAAGTTTATTT | |
| | CATCCTTATATGACGAGGAAAGAAAAGAAGAGAGAAATAATATCTTAA | |
| | CTTCTAAATCAGTCGCGTCAACTTTCTCGGCTAAGAAAGTGAGCACTA | |
| | TCATTTCGCAGACCATGTCATGAGTGCCGACTTGCCATATCTTATTAT | |
| | ATTCTTATTTATTTAATTATAATCCCATTGCAATACGTCTATTCTATC | |
| | ATGGCCTGCCACTAACGCTCCGTCTAACGTCGTTAAGCCATTGTCATA | |
| | AGCGGCTGCTCAAAACTCTTCCCGGTGGAGGCGAGGCGTTAACGGCGT | |
| | CTACAAATCTAACGGCCACCAACCATCCAGCCGCCTCTCGAAAGCTCC | |
| | GCTCCGATCGCGGAAATTGCGTGGCGGAGACGAGCGGGCTCCTCTCAC | |
| | ACGGCCCGGAACCGTCACGGCACGGGTGGGGGATTCCTTCCCCAACCC | |
| | TCCCCACCTCTCCTCCCCCCGTCGCAGCCCATAAATACAGGGCCCTCC | |
| | GCGCCTCTTCCCACAATCTCACATCGTCTCATCGTTCGGAGCGCACAA | |
| | CCCCCGGGTTCCAAATCCAAATTGCTCTTCTCGCGACCCTCGGCGATC | |
| | CTTCCCCCGCTTCAAGGTACGGCGATCGTCTCCCCCGTCCTCTTGCCC | |
| | CATCTCCTCGCTCGGCGTGGTTTGGTGGTTCTGCTTGGTCTGTGGCTA | |
| | GGAACTAGGCTGAGGCGTTGACGAAATCATGCTAGATCCGCGTGTTTC | |
| | CTGATCGTGGGTGGCTGGGAGGTGGGGTTTTCGTGTAGATCTGATCGG | |
| | TTCCGCTGTTTATCCTGTCATGCTCATGTGATTTGTGGGGATTTTAGG | |
| | TCGTTTGTCCGGGAATCGTGGGGTTGCTTCTAGGCTGTTCGTAGATGA | |
| | GATCGTTCTCACGATCTGCTGGGTCGCTGCCTAGGTTCAGCTAGGTCT | |
| | GCCCTGTTTTTGGGTTCGTTTTCGGGATCTGTACGTGCATCTATTATC | |
| | TGGTTCGATGGTGCTAGCTAGGAACAAACAACTGATTCGTCCGATCGA | |
| | TTGTTTTGTTGCCATGTGCAAGGTTAGGTCGTTATCTGATTGCTGTAG | |
| | ATCAGAGTAGAATAAGATCATCACAAGCTAGCTCTTGGGCTTATTATG | |
| | AATCTGCGTTTGTTGCATGATTAAGATGATTATGCTTTTTCTTATGCT | |
| | GCCGTTTGTATATGATGCGGTAGCTTTTAACTGAATAGCACACCTTTC | |
| | CTGTTTAGTTAGATTAGATTAGATTGCATGATAGATGAGGATATATGC | |
| | TGCTACATCAGTTTGATGATTCTCTGGTACCTCATAATCAACTAGCTC | |
| | ATGTGCTTAAATTGAAACTGCATGTGCCACATGATTAAGATGCTAAGA | |
| | TTGGTGAAGATATATACGCTGCTGTTCCTATAGGATCCTGTAGCTTTT | |
| | ACCTGGTCAACATGCATCGTCCTGTTATGGATAGATATGCATGATAGA | |
| | TGAAGATATGTACTGCTACAATTTGATGATTCTTTTGTGCACCTGATG | |
| | ATCATGCATGCTCTTTGCCCTTACTTTGATATACTTGGATGATGGCAT | |
| | GCTTAGTACTAATGATGTGATGAACACACATGACCTGTTGGTATGAAT | |
| | ATGATGTTGCTGTTTGCTTGTGATGAGTTCTGTTTGTTTACTGCTAGG | |
| | CACTTACCCTGTTGTCTGGTTCTCTTTTGCAGAATGGAGTCATCAACA | |
| | AAAGAACAAGAACAACAACACATAATCCTCCTCCCATTTTTAGCACAA | |
| | GGCCATCTCCGACCATTCCTCCACCTCGCTCACCGCCTTCTCTCTCTA | |
| | ACACCCTTCAATCTCTCTCTCCTCACTACCCCTTTAAACGCCGCTAAT | |
| | CTCCGCCGTCAATCCGACAACTTCAACATCAACCTTAACATCATCGAA | |
| | CTCCCCTTCACTAGCACCGACCACGGTGTCCCACCCAACACCGAAAAT | |
| | ACCGATAAACTCTCATTAACCTCCATCATCAAACTCTTCCATGCATCC | |
| | ACTTCACTCGAACCTCATGTACGTGACTACCTCACGCGCCACCACCTG | |
| | CACGACCCACCCGTGTGCATAATCTTCGACGTGTTCTTGGGCTGGGCC | |
| | GATAACGTGGCCCGATCAGTTGGGTCCACCGGAATCTGCTTCAACACT | |
| | GGTGGGGCCTACGGTGTCGGTGCGTATGTTTCAATCTGGAGCAATCTA | |
| | CCTCACCGGAATGTCGGCGATGATGAGGAGTTTTCATTGGCTGGTTTC | |
| | CCGGAAGATCGGAAACTCCGACGTAATCAGCTTCATCGGTTTTTAAGG | |
| | TTTGCTGATGGGTCCGATGAATGGTCGAGGTTTTTCCAACCTCAGATT | |
| | AAATCATCCTTGAATTGTTCTGGGTGGTTGTGTAATTCCATCGAGGAG | |
| | ATTGAACCGTTAGGGTTTCAAGTTTTTAGGAATTTCACGAAATCTCCT | |
| | ATTTGGGGAATTGGTCCTTTGATTATGACATCTTCGAAAAAAGATGAT | |
| | GATGAGAAGGAAGAGGCTTGTTTGAGGTGGTTGAATCAATTTGAAAAT | |
| | GACTCAGTTTTGTACATTTGTTTCGGGTCACAGAATACAGTGACTCCG | |
| | ATTCAGATGATGGAGTTAGCGAAAGGTTTGGAGGAGAGTAAGATCCCG | |
| | TTCTTGTGGGTGATTCGGCCGCCATTCGGGTTCGATTTCAATGGGGAG | |
| | TTTAAACCCGAATGGCTGCCTGAGAAGTTTGAGGAAAGAATGATGGAG | |
| | AAAAAACAGGGGATGTTGGTGAGAGATTGGGTGCCTCAGTTGGATATT | |
| | TTGAGGCATGAGGCTACTGGTGGATTTTTGAGTCATTGTGGTTGGAAT | |
| | TCGGTATTGGAAGGTTTAAGAGAAGGAGTGCCAATACTCGGGTGGCCG | |
| | TTGGCGGCCGAGCAAGCGTATAATTCAAAGATGATGGTGGAGGAAATG | |
| | GGGGTGGCGGTCGAGTTGACGAGGGGGTTGGAAGGGGAGGTGAAGAAA | |
| | GAGTGGGTGAAGAGTGTGGTGGAAATGGTGTTGGATAGAAAGGAGGGG | |
| | AGTTGTGGGTGGGAAATGAAGAAGAAAGCTGTTGAGATTGGGGAGAAA | |
| | TTGAAGGATGCTTGGAAAATTGAGGGAGATTACAAAGGATCTTCTGTT | |
| | AAGGCAATGGATAATTTTGTAGAGTTTATAGTTTCTTGTAGGGGAAAG | |
| | AAATCTAAAGAAGATGCTTGAGCTTCTCTAGCTAGAGTCGATCGACAA | |
| | GCTCGAGTTTCTCCATAATAATGTGTGAGTAGTTCCCAGATAAGGGAA | |
| | TTAGGGTTCCTATAGGGTTTCGCTCATGTGTTGAGCATATAAGAAACC | |
| | CTTAGTATGTATTTGTATTTGTAAAATACTTCTATCAATAAAATTTCT | |
| | AATTCCTAAAACCAAAATCCAGTACTAAAATCCAGATCGCTCAGATGA | |
| | GACCGAGGATGCACATGTGACCGAGGGACACGAA | |

### Sequence Optimization

The present disclosure recognizes that in some embodiments, it may be desirable to modify (e.g., optimize) the sequence of a gene of interest and/or of a polypeptide encoded by a gene of interest, for example in order to improve its function as described herein.

For example, in some embodiments, nucleic acid sequences provided by and/or utilized in accordance with the present disclosure are codon-optimized and/or otherwise adjusted, including by being associated with one or more appropriate regulatory elements, to improve and/or regulate expression in plant cells or organelles (e.g., chloroplasts) thereof.

Alternatively or additionally, in some embodiments, polypeptide sequences provided and/or utilized in accordance with the present disclosure may be modified relative to an appropriate reference. For example, in some embodiments, a luciferase polypeptide provided and/or utilized in accordance with the present disclosure is modified from a reference luciferase (e.g., as disclosure here) so that its ability to utilize chlorophyll and/or chlorophyllides as a substrate is enhanced.

### Directed Mutagenesis

In some embodiments, to increase the activity of a given enzyme (e.g., a luciferase or a metabolic enzyme), specific mutations are induced, typically leading to a change in its catalytic site, (e.g., the active site often considered crucial for its enzymatic reaction). In some embodiments, these mutations can be deliberately chosen through careful examination of the polypeptide structure and activity, sometimes called evolution by rational design. Alternatively, in some embodiments, the mutations can also be random, driven through a process called directed evolution; wherein random mutations are introduced with multiple rounds of error-prone amplification of the DNA sequence. In some embodiments, such amplification of a DNA sequence may occur through a system such as error-prone polymerase chain reaction. In some embodiments, such amplification of a DNA sequence may occur through introduction of the gene into a mutagenic vector and/or organism (e.g., XL1 Red). Those skilled in the art will recognize there are multiple suitable methods for mediating error-prone DNA amplification. In some embodiments, this methodology results in a mutant library from which we can test the activity and select the most active and/or desirable variants from the pool of available mutants. This process allows the testing of many thousands of iterations in parallel, coupling the power of error-prone amplification with stringent selection to harness directed evolution and to create desired and yet difficult to predict mutant enzymes.

### Fusion and Chimeric Polypeptides

In some embodiments, sequences of individual genes of interest coding for enzymes of interest are optimized through the addition of heterologous polypeptide domains, wherein domains are combined to create “fusion polypeptides”. In some embodiments, instead of inserting at least two genes, each with its own promoter, coding for at least two enzymes involved in the same or related pathways, a single coding sequence can be inserted. In some embodiments, that sequence comprises the first gene sequences without its stop codon, an optional linker region (e.g., a string of 10-12 codons coding for neutral amino acids), followed by the coding sequence of at least a second gene of interest, wherein the final coding sequence comprises a stop codon. In some embodiments, this method can result in a single reading frame and the expression of a single fusion polypeptide. In some embodiments, this methodology provides certain advantages, e.g., a fusion polypeptide comprising at least two polypeptides may bring their respective catalytic sites into closer physical proximity, increasing the overall reaction speed. In some embodiments, this method can be used to create fusion polypeptides combining 3 or more polypeptides (e.g., at least 3 polypeptides, at least 4 polypeptides, at least 5 polypeptides, at least 6 polypeptides), however, this may induce steric hindrance.

### Engineered Chloroplast-Containing Photosynthetic Organisms

In some embodiments, a CPOS described herein comprises one or more genes (e.g., one or more heterologous genes) and/or polypeptides associated with a betalain biosynthesis pathway. In some embodiments, engineered CPOS (e.g., plant) comprise one or more genes (e.g., one or more heterologous genes) and/or polypeptides associated with a betalain biosynthesis pathway and exhibit one or more colors and/or patterns (e.g., non-natural colors and/or patterns).

In some embodiments, provided technologies are applied to one or more chloroplast-containing photosynthetic organisms (CPOS). For example, in some embodiments, provided technologies are applied to one or more plants (e.g., one or more indoor plants, one or more outdoor plants, one or more ornamental plants). In some embodiments, provided technologies are applied to one or more ornamental plants (e.g., one or more indoor and/or one or more outdoor ornamental plants).

In some embodiments, provided technologies are applied to one or more indoor plants. In some embodiments, provided technologies are applied to one or more outdoor plants. In some embodiments, provided technologies are applied to one or more ornamental plants (e.g., one or more indoor ornamental plants and/or to one or more outdoor ornamental plants).

Epipremnum aureum (Pothos, Golden Pothos, or Devil's Ivy)

In some embodiments, provided technologies are applied to Epipremnum aureum, which is typically considered to be an ornamental plant, and specifically an indoor ornamental plant. Epipremnum aureum is a species of flowering plant in the arum family Araceae, native to Mo'orea in the Society Islands of French Polynesia. The species is a popular houseplant in temperate regions, but has also become naturalized in tropical and sub-tropical forests worldwide, including northern Australia, Southeast Asia, South Asia, the Pacific Islands and the West Indies (where it has caused severe ecological damage in some cases). The plant has a multitude of common names including golden pothos, pothos, Ceylon creeper, hunter's robe, ivy arum, silver vine, Solomon Islands ivy, marble queen, devil's vine, devil's ivy, and taro vine.

In some embodiments, Epipremnum aureum is particularly amenable as an indoor ornamental house plant as it is considered hardy, is often difficult to kill, and generally stays green even when kept in the dark. In some embodiments, Epipremnum aureum is an evergreen vine growing to 20 m (66 ft) tall, with stems up to 4 cm (2 in) in diameter, climbing by means of aerial roots which adhere to surfaces. In some embodiments, Epipremnum aureum leaves are alternate, heart-shaped, entire on juvenile plants, but irregularly pinnatifid on mature plants, up to 100 cm (39 in) long and 45 cm (18 in) broad; juvenile leaves may be smaller, typically under 20 cm (8 in) long. In some embodiments, Epipremnum aureum rarely flowers without artificial hormone supplements, but when it does, the flowers are produced in a spathe up to 23 cm (9 in) long. In some embodiments, Epipremnum aureum produces trailing stems when it climbs up trees and/or other structures, and these trailing stems can take root when they reach the ground and grow along it. In some embodiments, leaves on trailing stems grow up to 10 cm (4 in) long and are reminiscent of the leaves seen on Epipremnum aureum when it is cultivated as a potted plant. In some embodiments, Epipremnum aureum can be considered a popular houseplant with numerous cultivars selected for leaves with white, yellow, or light green variegation. In some embodiments, Epipremnum aureum can be used in decorative displays in shopping centers, offices, and/or other public locations in part because it requires little care and is also attractively leafy. In certain tropical countries, Epipremnum aureum may be found in parks and gardens and tends to grow naturally. In some embodiments, as an indoor plant, Epipremnum aureum can reach more than 2 m in height, particularly when given adequate support (e.g., a structure to climb), but as an indoor plant, Epipremnum aureum generally fails to develop adult-sized leaves. In some embodiments, Epipremnum aureum can be considered a “shady” plant, and optimal growth conditions may be achieved by providing indirect light. In some embodiments, Epipremnum aureum can tolerate an intense luminosity, but long periods of direct sunlight may burn leaves. In some embodiments, Epipremnum aureum thrives in temperature to tropical temperatures between 17 and 30° C. (63 and 86° F.). In some embodiments, Epipremnum aureum only requires watering when the soil feels dry to the touch. In some embodiments, Epipremnum aureum tolerates and may be benefited by supplemental fertilizers and may grow rapidly in hydroponic culture. In some embodiments, Epipremnum aureum is sometimes used in aquariums, e.g., it may be placed on top of the aquarium and allowed to grow roots into the water, this may be beneficial to the plant and the aquarium as Epipremnum aureum may absorb soluble nitrates and use them for growth.

In some embodiments, Epipremnum aureum may be considered as toxic to cats and dogs due to the presence of insoluble raphides. In some embodiments, care should be taken to ensure that Epipremnum aureum is not consumed by pets. In some embodiments, symptoms of Epipremnum aureum consumption may include oral irritation, vomiting, and/or difficulty in swallowing. In some embodiments, potentially due to calcium oxalate within Epipremnum aureum, it may be considered mildly toxic to humans as well. In some embodiments, possible side effects from consumption of Epipremnum aureum are atopic dermatitis (eczema) as well as burning and/or swelling of the region inside of and surrounding the mouth. In some embodiments, excessive contact with Epipremnum aureum may also lead to general skin irritation.

### Other Ornamental Plants

In some embodiments, technologies described herein comprise an engineered ornamental plant that is of the family Araceae. In some embodiments, an ornamental plant can be but is not limited to the genera Aglaonema, Alocasia, Amorphophallus, Anthurium, Caladium, Colocasia, Dieffenbachia, Epipremnum, Monstera, Philodendron, Rhaphidophora, Scindapsus, Spathiphyllum, Syngonium, Xanthosoma, Zamioculcas, and Zantedeschia.

In some embodiments, technologies described herein comprise an engineered ornamental plant that is of the class Polypodiopsida (e.g., a fern) In some embodiments, an engineered ornamental plant can be but is not limited to the species Cyathea cooperi, Adiantum hispidulum, Adiantum raddianum, Adiantum tenerum, Aglaomorpha coronans, Asplenium antiquum, Asplenium nidus, Blechnum gibbum, Davallia fejeensis, Didymochlaena truncatula, Dryopteris erythrosora, Humata tyermanii, Microsorum diversifolium, Nephrolepis cordifolia, Nephrolepis exaltata, Pellaea rotundifolia, Phlebodium aureum mandaianum, Platycerium bifurcatum, Polypodium formosanum, Pteris cretica, Pteris ensiformis, and Pteris quadriaurita.

In some embodiments, technologies described herein comprise an engineered plant that is of the family Marantaceae (e.g., Calatheas). In some embodiments, an engineered ornamental plant can be but is not limited to the species Calathea ornata, Calathea rufibarba, Calathea orbifolia, Calathea roseopicta, Calathea zebrine, Calathea lancifolia, Calathea warscewiczii, Calathea louisae, Calathea veitchiana, Calathea picturata, Calathea ecuadoriana, Calathea gandersii, Calathea curaraya, Calathea libbyana, Calathea hagbergii, Calathea roseobracteata, Calathea paucifolia, Calathea ischnosiphonoides, Calathea multicinta, Calathea latrinotecta, Calathea dodsonii, Calathea anulque, Calathea lanicaulis, Calathea petersenii, Calathea pluriplicata, Calathea plurispicata, Calathea pallidicosta, Calathea congesta, and Calathea utilis.

Engineering Ornamental Plants and/or Microbes

In some embodiments, the present disclosure provides technologies that comprise and/or utilize engineered CPOS (e.g., plants and/or algae) and/or microbes (e.g., that support or interact with plants, such as by occupying the rhizome) including, for example, chemically engineered, environmentally engineered, and/or genetically engineered CPOS (e.g., plants and/or algae) and/or microbes (e.g., that support or interact with plants, such as that occupy the rhizome).

In some embodiments, chemical engineering may be or comprise exposure to one or more particular chemical agents (e.g., nutrients, mutagens, etc).

In some embodiments, environmental engineering may be or comprise exposure, maintenance, and/or cultivation under a specified set of conditions (e.g., light, temperature, pressure, pH, etc) and/or involving one or more particular manipulations (e.g., grafting, traditional cloning, re-potting, etc).

In some embodiments, genetic engineering may be or comprise introducing one or more genetic modifications (e.g., insertions, deletions, and/or alterations of one or more particular sequences—e.g., genes). In some embodiments, genetic modification may involve and/or be accomplished through performance of one or more of transformation, transduction, and/or other introduction of a transgene or other heterologous nucleic acid sequence; disruption and/or interference with expression of one or more genetic sequences (e.g., gene knockout, gene knockdown, etc), induction and/or amplification of expression of one or more genetic sequences, alteration (e.g., by mutagenesis such as targeted or random mutagenesis), etc. In some embodiments, genetic engineering may involve one or more of selective breeding, and/or directed evolution.

In some embodiments, a CPOS (or related microbe) is genetically engineered through a process of selective breeding and/or directed evolution across multiple generations using at least one sufficiently selective pressure, followed by optional mutation identification (e.g., genotyping), and phenotypic analysis.

In some embodiments, a CPOS (or related microbe) is genetically engineered through a process of random mutagenesis followed by screening for a trait of interest, optional mutation identification (e.g., genotyping), and phenotypic analysis.

In some embodiments, a CPOS (or related microbe) is genetically engineered through a process of directed mutagenesis, followed by optional mutation verification (e.g., genotyping), and phenotypic analysis.

In some embodiments, a CPOS (or related microbe) is genetically engineered through a process of transgene introduction, followed by optional mutation verification (e.g., genotyping), and phenotypic analysis.

In some embodiments, CPOS (or related microbe) is genetically engineered by introduction of a vector into such CPOS (or related microbe) (e.g., into a cell or spore thereof). In some embodiments, a vector suitable for plant transformation is generated, is optionally verified through any appropriate technology (e.g., sequencing, PCR, gel electrophoresis), and is then inserted into a plant genome. In some embodiments, insertion into a plant genome can be accomplished through 1) Agrobacterium tumefaciens (A. tumefaciens) mediated gene insertion, or 2) biolistic mediated gene insertion (DNA bombardment method).

In some embodiments, A. tumefaciens insertion may be an appropriate methodology to use when a working protocol exists. In some embodiments, insertion of a gene into a plant comprises: 1) A. tumefaciens transformation by electroporation, 2) selection of viable clones, and 3) plant infection; in some embodiments this process can allow for relatively high transformation efficiencies. In some embodiments, binary plasmids are utilized. In some embodiments, binary plasmids are compatible with A. tumefaciens-based transformations. In some embodiments, binary plasmids are utilized as part of a golden gate DNA assembly system.

In some embodiments, a biolistic particle delivery system, or “gene gun” approach is utilized to mediate gene insertion into a plant. In some embodiments, such an approach utilizes DNA-coated gold particles to deliver a vector of interest to cells, integrating all or at least a portion of the vector (e.g., a coding sequence) inside of a plant genome (e.g., any endogenous store of genetic material, e.g., DNA of the mitochondria, chloroplast, and/or nucleus). In some embodiments, such an approach creates an artificial chromosome. In some embodiments, an artificial chromosome is stably inherited through multiple generations. In some embodiments, a biolistic particle delivery system is utilized when no efficient A. tumefaciens mediated transformation protocol is available for a particular target species of plant. In some embodiments, a biolistic approach is preferential to A. tumefaciens-based transformations due to an inherent ability of biolistic introduction to target not only nuclear DNA, but also mitochondrial and/or chloroplastic DNA. In some embodiments, a biolistic approach may be preferential due to an inherent ability to insert lower copy numbers (e.g., 1 copy), potentially reducing the odds of transgene silencing by endogenous defense mechanisms.

In some embodiments, a CPOS is engineered through nuclear genome integration of our gene(s) containing a chloroplast localization signal, for its transport to the compartment containing the chlorophyll biosynthetic pathway.

In some embodiments, a CPOS is engineered through chloroplast genome integration of a heterologous or otherwise engineered gene(s) as described herein.

In some embodiments, an engineered CPOS (e.g., plant) expresses one or more CYP76AD1 polypeptides. In some embodiments, an engineered CPOS (e.g., plant) expresses one or more DODA polypeptides. In some embodiments, an engineered CPOS (e.g., plant) expresses one or more cDOPA5GT polypeptides. In some embodiments, an engineered CPOS (e.g., plant) expresses one or more DOPA polypeptides. In some embodiments, an engineered CPOS (e.g., plant) expresses one or more ADHα polypeptides. In some embodiments, an engineered CPOS (e.g., plant) expresses one or more AroG^{L175Q} polypeptides. In some embodiments, an engineered CPOS (e.g., plant) expresses one or more CYP76AD1 polypeptides, one or more DODA polypeptides, one or more cDOPA5GT polypeptides, one or more DOPA polypeptides, one or more ADHα polypeptides, one or more AroG^{L175Q} polypeptides, or any combination thereof. In some embodiments, a polypeptide described herein is a heterologous polypeptide.

In some embodiments, an engineered CPOS (e.g., plant) comprises one or more pigment-producing polypeptides, fragments thereof, or characteristic portions thereof.

In some embodiments, an engineered CPOS (e.g., plant) produces betalains. In some embodiments, an engineered CPOS (e.g., plant) produces betacyanins. In some embodiments, an engineered CPOS (e.g., plant) produces betaxanthins. In some embodiments, an engineered CPOS (e.g., plant) produces one or more polypeptides comprised in a betalain biosynthesis pathway. In some embodiments, an engineered CPOS (e.g., plant) produces one or more genes encoding one or more polypeptides comprised in a betalain biosynthesis pathway. In some embodiments, an engineered CPOS (e.g., plant) produces one or more CYP76AD1 polypeptides. In some embodiments, an engineered CPOS (e.g., plant) produces one or more DODA polypeptides. In some embodiments, an engineered CPOS (e.g., plant) produces one or more DOPA5GT polypeptides. In some embodiments, an engineered CPOS (e.g., plant) produces one or more DOPA polypeptides. In some embodiments, an engineered CPOS (e.g., plant) produces one or more ADHα polypeptides. In some embodiments, an engineered CPOS (e.g., plant) produces one or more AroG^{L175Q} polypeptides. In some embodiments, an engineered CPOS (e.g., plant) produces one or more CYP76AD1 polypeptides, one or more DODA polypeptides, one or more DOPA5GT polypeptides, one or more DOPA polypeptides, one or more ADHα polypeptides, one or more AroG^{L175Q} polypeptides, or any combination thereof. In some embodiments, a polypeptide described herein is a heterologous polypeptide.

In some embodiments, an engineered CPOS (e.g., plant) expresses one or more genes encoding one or more CYP76AD1 polypeptides. In some embodiments, an engineered CPOS (e.g., plant) expresses one or more genes encoding one or more DODA polypeptides. In some embodiments, an engineered CPOS (e.g., plant) expresses one or more genes encoding one or more cDOPA5GT polypeptides. In some embodiments, an engineered CPOS (e.g., plant) expresses one or more genes encoding one or more DOPA polypeptides. In some embodiments, an engineered CPOS (e.g., plant) expresses one or more genes encoding one or more ADHα polypeptides. In some embodiments, an engineered CPOS (e.g., plant) expresses one or more genes encoding one or more CYP76AD1 polypeptides, one or more DODA polypeptides, one or more cDOPA5GT polypeptides, one or more DOPA polypeptides, one or more ADHα polypeptides, or any combination thereof. In some embodiments, a polypeptide or gene described herein is a heterologous polypeptide or gene.

In some embodiments, an engineered CPOS (e.g., plant) expresses one or more heterologous CYP76AD1 polypeptides, one or more heterologous DODA polypeptides, and/or one or more heterologous cDOPA5GT polypeptides. In some embodiments, an engineered CPOS (e.g., plant) expresses one or more genes encoding one or more heterologous CYP76AD1 polypeptides, one or more heterologous DODA polypeptides, and one or more heterologous cDOPA5GT polypeptides.

In some embodiments, an engineered CPOS (e.g., plant) expresses one or more heterologous CYP76AD1 polypeptides, one or more heterologous DODA polypeptides, and/or one or more heterologous DOPA polypeptides. In some embodiments, an engineered CPOS (e.g., plant) expresses one or more genes encoding one or more heterologous CYP76AD1 polypeptides, one or more heterologous DODA polypeptides, and one or more heterologous DOPA polypeptides.

In some embodiments, an engineered CPOS (e.g., plant) expresses one or more heterologous CYP76AD1 polypeptides, one or more heterologous DODA polypeptides, and/or one or more heterologous UDP92A1 polypeptides. In some embodiments, an engineered CPOS (e.g., plant) expresses one or more genes encoding one or more heterologous CYP76AD1 polypeptides, one or more heterologous DODA polypeptides, and one or more heterologous UDP92A1 polypeptides.

In some embodiments, an engineered CPOS (e.g., plant) expresses one or more heterologous ADHα polypeptides, one or more heterologous CYP76AD1 polypeptides, one or more heterologous DODA polypeptides, and/or one or more heterologous DOPA polypeptides. In some embodiments, an engineered CPOS (e.g., plant) expresses one or more heterologous ADHα polypeptides, one or more heterologous CYP76AD1 polypeptides, one or more heterologous DODA polypeptides, and one or more heterologous DOPA polypeptides.

In some an engineered CPOS (e.g., plant) is or comprises an engineered ornamental plant of the family Araceae. In some embodiments, an ornamental plant can be but is not limited to the genera Aglaonema, Alocasia, Amorphophallus, Anthurium, Caladium, Colocasia, Dieffenbachia, Epipremnum, Monstera, Philodendron, Rhaphidophora, Scindapsus, Spathiphyllum, Syngonium, Xanthosoma, Zamioculcas, and Zantedeschia.

In some embodiments, an engineered CPOS (e.g., plant) is or comprises a plant in the genera Epipremnum. In some embodiments, an engineered CPOS (e.g., plant) is or comprises Epipremnum aureum.

In some embodiments, an engineered CPOS (e.g., plant) is or comprises a plant in the genera Monstera.

### Pigments

Stable heterologous production of betalains has been reported in a limited number of plant species and microbes (see, e.g., WO2017122189A1, e.g., Polturak G, Aharoni A. Advances and future directions in betalain metabolic engineering. New Phytol. 2019 December; 224 (4): 1472-1478., and e.g., He, Y., et al. A reporter for noninvasively monitoring gene expression and plant transformation. Hortic Res 7, 152 (2020), which are hereby incorporated by reference herein in their entirety). Among other things, the present disclosure provides an insight for expression and/or production of betalain in certain CPOS (e.g., family Araceae, e.g., genera Epipremnum, e.g., Epipremnum aureum).

In some embodiments, an engineered CPOS (e.g., plant) produces and/or expresses a pigment. In some embodiments, an engineered CPOS (e.g., plant) produces and/or expresses a pigment in at least one part of the engineered plant. In some embodiments, at least one part of an engineered CPOS (e.g., plant) produces and/or expresses a pigment.

In some embodiments, an engineered CPOS (e.g., plant) described herein produces and/or expresses a pigment in an organelle, a cell, a tissue, or an organ. In some embodiments, an engineered CPOS (e.g., plant) described herein produces and/or expresses a pigment in a chloroplast and/or a vacuole. In some embodiments, an engineered CPOS (e.g., plant) described herein produces and/or expresses a pigment in an epidermal cell, a guard cell, a mesophyll cell, a hypodermal cell, and/or a sub-epidermal cell. In some embodiments, an engineered CPOS (e.g., plant) described herein produces and/or expresses a pigment in dermal tissue. In some embodiments, an engineered CPOS (e.g., plant) described herein produces and/or expresses a pigment in a leaf, node, a stem, and/or a root.

In some embodiments, an engineered CPOS (e.g., plant) described herein produces and/or expresses a pigment in an organelle, a cell, a tissue, or an organ, where the production and/or expression of the pigment is attributable to the expression of a polynucleotide construct described herein.

In some embodiments, a pigment is or comprises betalain. In some embodiment, a pigment is or comprises betacyanin. In some embodiment, a pigment is or comprises betanin.

### Pigmentation Pattern

In some embodiments, pigmentation pattern refers to any spatial distribution of one or more pigments in an engineered CPOS (e.g., plant). In some embodiments, a pigmentation pattern may comprise one or more uniform regions. In some embodiments, a pigmentation pattern may comprise one or more non-uniform regions. In some embodiments, the distribution may appear organized. In some embodiments, the distribution may appear non-organized.

In some embodiments, a pigmentation pattern may comprise contiguous or non-contiguous regions that differ in colorimetric and/or biochemical attributes. In some embodiments, a pigmentation pattern may comprise contiguous or non-contiguous regions that differ in hue, saturation, brightness, reflectance, pigment concentration, color, color pattern, or any combination thereof.

In some embodiments, a pigmentation pattern described herein does not require any particular geometry, periodicity, symmetry, or regularity. In some embodiments, a pigmentation pattern described herein encompasses uniform, organized, and non-organized distributions of pigment, color, and/or color pattern

Colors and/or Color Patterns

In many embodiments, the present disclosure relates to engineering non-natural pigments into CPOS (e.g., plants).

In some embodiments, CPOS (e.g., plants) exhibit one or more colors and/or patterns. In some embodiments, a color and/or pattern is uniform. In some embodiments, an engineered CPOS (e.g., plant) is characterized by a uniform pink, uniform red, uniform purple, or uniform color variant thereof (e.g., pink color variant, red color variant, purple color variant). In some embodiments, an engineered CPOS (e.g., plant) is characterized by a uniform light red pink color, uniform medium red pink color, uniform pink color, uniform light blue pink color, uniform medium blue pink color, uniform dark blue pink color, uniform orange red color, uniform light red color, uniform medium red color, uniform dark red color, uniform medium purple red color, uniform dark purple red color, uniform brown red color, uniform medium brown purple color, uniform dark brown purple color, uniform medium purple color, uniform dark purple color, uniform light violet color, uniform medium violet color, uniform dark violet color, uniform light blue violet color, uniform medium blue violet color, uniform dark blue violet color, uniform light violet blue color, uniform medium violet blue color, uniform dark violet blue color, uniform red color, uniform violet color, uniform crimson color, uniform scarlet color, uniform burgundy color, uniform cherry red color, uniform tomato red color, uniform fire engine red color, uniform ruby color, uniform maroon color, uniform brick red color, uniform raspberry color, uniform vermilion color, uniform cardinal red color, uniform rose red color, uniform blood red color, uniform salmon red color, or a combination thereof.

In some embodiments, a color and/or pattern is non-uniform. In some embodiments, an engineered CPOS (e.g., plant) is characterized by a non-uniform pink, non-uniform red, non-uniform purple, or non-uniform color variant thereof (e.g., pink color variant, red color variant, purple color variant). In some embodiments, an engineered CPOS (e.g., plant) is characterized by a non-uniform light red pink color, non-uniform medium red pink color, non-uniform pink color, non-uniform light blue pink color, non-uniform medium blue pink color, non-uniform dark blue pink color, non-uniform orange red color, non-uniform light red color, non-uniform medium red color, non-uniform dark red color, non-uniform medium purple red color, non-uniform dark purple red color, non-uniform brown red color, non-uniform medium brown purple color, non-uniform dark brown purple color, non-uniform medium purple color, non-uniform dark purple color, non-uniform light violet color, non-uniform medium violet color, non-uniform dark violet color, non-uniform light blue violet color, non-uniform medium blue violet color, non-uniform dark blue violet color, non-uniform light violet blue color, non-uniform medium violet blue color, non-uniform dark violet blue color, non-uniform red color, non-uniform violet color, non-uniform crimson color, non-uniform scarlet color, non-uniform burgundy color, non-uniform cherry red color, non-uniform tomato red color, non-uniform fire engine red color, non-uniform ruby color, non-uniform maroon color, non-uniform brick red color, non-uniform raspberry color, non-uniform vermilion color, non-uniform cardinal red color, non-uniform rose red color, non-uniform blood red color, non-uniform salmon red color, or a combination thereof.

In some embodiments, a color and/or pattern comprising one or more colors. In some embodiments, an engineered CPOS (e.g., plant) is characterized by one or more colors. In some embodiments, one or more colors is or comprises pink, red, purple, or a color variant thereof (e.g., pink color variant, red color variant, purple color variant). In some embodiments, an engineered CPOS (e.g., plant) is characterized by one or more colors selected from pink, red, purple, or a color variant thereof (e.g., pink color variant, red color variant, purple color variant). In some embodiments, an engineered CPOS (e.g., plant) is characterized by one or more colors comprising light red pink, medium red pink, pink, light blue pink, medium blue pink, dark blue pink, orange red, light red, medium red, dark red, medium purple red, dark purple red, brown red, medium brown purple, dark brown purple, medium purple, dark purple, light violet, medium violet, dark violet, light blue violet, medium blue violet, dark blue violet, light violet blue, medium violet blue, dark violet blue, red, violet, crimson, scarlet, burgundy, cherry red, tomato red, fire engine red, ruby, maroon, brick red, raspberry, vermilion, cardinal red, rose red, blood red, or salmon red color.

In some embodiments, a color described herein is categorized relative to an appropriate reference. In some embodiments, a color described herein categorized relative to a standard color description. In some embodiments, a color described herein is defined by a UPOV group (see, e.g., upov.int/edocs/tgpdocs/en/tgp_14.pdf, the contents of which is hereby incorporated by reference herein in its entirety). In some embodiments, a color described herein is defined by a RHS number (see, e.g., hedgexpress.co.uk/rhs-colour-chart/, the contents of which is hereby incorporated by reference herein in its entirety). In some embodiments, a color described herein is defined by a sRGB number (see, e.g., internationallilacsociety.org/wp-content/uploads/2019/12/Lilac-Color-Groups-by-RHS-columns.pdf, the contents of which is hereby incorporated by reference herein in its entirety). In some embodiments, a color described herein is defined by a UPOV group, a RHS number, sRGB number, or any combination thereof.

In some embodiments, a pattern described herein is categorized relative to an appropriate reference. In some embodiments, a pattern r described herein categorized relative to a standard color description. In some embodiments, a pattern described herein is defined by a UPOV group (see, e.g., upov.int/edocs/tgpdocs/en/tgp_14.pdf, the contents of which is hereby incorporated by reference herein in its entirety). In some embodiments, a pattern described herein is defined by a RHS number (see, e.g., hedgexpress.co.uk/rhs-colour-chart/, the contents of which is hereby incorporated by reference herein in its entirety). In some embodiments, a pattern described herein is defined by a sRGB number (see, e.g., internationallilacsociety.org/wp-content/uploads/2019/12/Lilac-Color-Groups-by-RHS-columns.pdf, the contents of which is hereby incorporated by reference herein in its entirety). In some embodiments, a pattern described herein is defined by a UPOV group, a RHS number, sRGB number, or any combination thereof.

In some embodiments, a color and/or pattern is observed in one or more parts of a CPOS (e.g., plant). In some embodiments, one or more part of a CPOS (e.g., plant) are the same. In some embodiments, one or more part of a CPOS (e.g., plant) are different.

In some embodiments, an engineered CPOS (e.g., plant) exhibits a level of pink pigmentation in one of more plant parts as compared to an appropriate reference (e.g., a non-engineered CPOS (e.g., plant)). In some embodiments, an engineered CPOS (e.g., plant) exhibits a level of red pigmentation in one of more plant parts as compared to an appropriate reference (e.g., a non-engineered CPOS (e.g., plant)). In some embodiments, an engineered CPOS (e.g., plant) exhibits a level of purple pigmentation in one of more plant parts as compared to an appropriate reference (e.g., a non-engineered CPOS (e.g., plant)). In some embodiments, an engineered CPOS (e.g., plant) exhibits a level of a color variant pigmentation thereof (e.g., pink color variant pigmentation, red color variant pigmentation, purple color variant pigmentation) in one of more plant parts as compared to an appropriate reference (e.g., a non-engineered CPOS (e.g., plant)).

In some embodiments, an engineered CPOS (e.g., plant) exhibits a level of pink pigmentation, red pigmentation, purple pigmentation, or a color variant pigmentation thereof (e.g., pink color variant pigmentation, red color variant pigmentation, purple color variant pigmentation), or any combination thereof in one of more plant parts as compared to an appropriate reference (e.g., a non-engineered CPOS (e.g., plant)).

In some embodiments, a color and/or pattern is as observed in one or more Figures of the Drawing included herewith.

In some embodiments, a color and/or pattern is observed in one or more parts of a CPOS (e.g., plant) over time. In some embodiments, a color and/or pattern is observed in one or more parts of a CPOS (e.g., plant) by a certain time and/or day (e.g., minutes, hours, days, weeks, months).

In some an engineered CPOS (e.g., plant) is or comprises an engineered ornamental plant of the family Araceae. In some embodiments, an ornamental plant can be but is not limited to the genera Aglaonema, Alocasia, Amorphophallus, Anthurium, Caladium, Colocasia, Dieffenbachia, Epipremnum, Monstera, Philodendron, Rhaphidophora, Scindapsus, Spathiphyllum, Syngonium, Xanthosoma, Zamioculcas, and Zantedeschia.

In some embodiments, an engineered CPOS (e.g., plant) is or comprises a plant in the genera Epipremnum. In some embodiments, an engineered CPOS (e.g., plant) is or comprises Epipremnum aureum.

In some embodiments, an engineered CPOS (e.g., plant) is or comprises a plant in the genera Monstera.

### Compositions

Among other things, the present disclosure provides compositions. In some embodiments, a composition comprises a genetically modified plant comprising a pink color phenotype, red color phenotype, purple color phenotype, a color variant thereof phenotype (e.g., pink color variant phenotype, red color variant phenotype, or purple color variant phenotype), or combination thereof. In some embodiments, a phenotype is due to expression and/or production of one or more betalains. In some embodiments, a phenotype is due to expression and/or production of one or more betacyanins. In some embodiments, a phenotype is due to expression and/or production of one or more betanins. In some embodiments, a phenotype is due to expression and/or production of one or more polypeptides comprised in a betalain biosynthesis pathway or one or more genes encoding one or more polypeptides comprised in a betalain biosynthesis pathway. In some embodiments, a phenotype is due to expression and/or production of one or more CYP76AD1 polypeptides, one or more DODA polypeptides, one or more DOPA5GT polypeptides, one or more DOPA polypeptides, one or more ADHα polypeptides, one or more AroG^{L175Q} polypeptides, or a combination thereof. In some embodiments, a phenotype is due to expression of one or more genes encoding one or more CYP76AD1 polypeptides, one or more DODA polypeptides, one or more DOPA5GT polypeptides, one or more DOPA polypeptides, one or more ADHα polypeptides, one or more AroG^{L175Q} polypeptides, or a combination thereof.

In some embodiments, a composition comprises a genetically modified plant comprising a pink color phenotype, a red color phenotype, a purple color phenotype, a color variant thereof phenotype (e.g., a pink color variant phenotype, a red color variant phenotype, or a purple color variant phenotype), or combination thereof.

In some embodiments, a composition comprises a genetically modified plant comprising a pink color phenotype, red color phenotype, purple color phenotype, a color variant thereof phenotype (e.g., pink color variant phenotype, red color variant phenotype, or purple color variant phenotype), or combination thereof, and an engineered microbe.

In some embodiments, a composition comprises an engineered microbe. In some embodiments, a composition comprises an engineered eukaryotic cell. In some embodiments, a composition comprises an engineered prokaryotic cell.

### Methods

In some embodiments, the present disclosure provides methods of using, making, and/or characterizing compositions described herein.

### Methods of Use

In some embodiments, provided herein are methods of using an engineered plant described herein. In some embodiments, an engineered plant of the present disclosure may be used as an indoor decorative element. In some embodiments, an engineered plant of the present disclosure may be used as an outdoor decorative element.

In some embodiments, an engineered plant described herein may be used to improve aesthetics in an environment. In some embodiments, an environment may comprise, but is not limited to: improving aesthetics of a home (e.g., a room in a home or in a dormitory) or in a public space (e.g., in an office, in a classroom, at a hospital, in a library, in a community center, in a museum, in a courthouse lobby, in a university hallway, in a café, in a restaurant, in a hotel lobby, in an office reception area, in a coworking space, in a train station waiting area, in an airport terminal, in a shopping mall corridor, in a public gallery, in a community garden pavilion, in a convention center atrium, at a park, in a zoo, in a botanical garden, in a theme park, or in a greenhouse). In some embodiments, an engineered plant described herein may be used to improve texture of an environment. In some embodiments, an engineered plant described herein may be used to improve color of an environment. In some embodiments, an engineered plant described herein may be used to improve vibrancy of an environment. In some embodiments, an engineered plant described herein may be used to increase home value (e.g., sale price of a home) or rental property value.

In some embodiments, an engineered plant described herein may be used to improve mental well-being of a human (e.g., wherein an individual is in the same space as an engineered plant). In some embodiments, well-being of a human may be improved by: reducing stress, reducing anxiety, reducing depression, enhancing mood, enhancing attention, enhancing productivity, enhancing creativity, enhancing mindfulness, enhancing sense of control, enhancing social connection, fostering a sense of meaning, or fostering responsibility.

In some embodiments, provided herein are methods of providing a pink color phenotype, red color phenotype, purple color phenotype, a color variant thereof phenotype (e.g., a pink color variant phenotype, a red color variant phenotype, or a purple color variant phenotype), or combination thereof to a plant to improve aesthetics in an environment.

### Methods of Characterizing

In some embodiments, compositions are characterized based upon their ability express certain compounds (e.g., betanin) in a CPOS. In some embodiments, increase in certain compounds (e.g., betanin) in a CPOS is assessed.

In some embodiments, provided herein are methods of characterizing engineered plants described herein. In some embodiments, provided herein are methods of characterizing a plant part in engineered plants described herein. In some embodiments, a plant part comprises or is an organelle, a cell, a tissue, or an organ. In some embodiments, an organelle is or comprises a chloroplast and/or a vacuole. In some embodiments, a cell is or comprises an epidermal cell, a guard cell, a mesophyll cell, a hypodermal cell, or a sub-epidermal cell. In some embodiments, a tissue is or comprises dermal tissue.

In some embodiments, an engineered plant of the present disclosure, or a part thereof, can be characterized visually (e.g., macroscopically). In some embodiments, visual characterization may comprise characterization based on hue, saturation, brightness, reflectance, pigment concentration, color, or color pattern.

In some embodiments, the present disclosure provides a method comprising visually characterizing an engineered plant described herein, wherein the characterization is or comprises observing or measuring a hue, a saturation, a brightness, a reflectance, and/or a pigment concentration. In some embodiments, the present disclosure provides a method comprising visually characterizing an engineered plant described herein, wherein the characterization is or comprises observing a pigmentation pattern.

In some embodiments, a pigmentation pattern is or comprises a color or a color pattern. In some embodiments, a color is or comprises a pink color, a red color, a purple color, or a brown color. In some embodiments, a color is or comprises a pink color variant, a red color variant, a purple color variant, or a brown color variant. In some embodiments, a color is or comprises a light red pink color, a medium red pink color, a pink color, a light blue pink color, a medium blue pink color, a dark blue pink color, an orange red color, a light red color, a medium red color, a dark red color, a medium purple red color, a dark purple red color, a brown red color, a medium brown purple color, a dark brown purple color, a medium purple color, a dark purple color, a light violet color, a medium violet color, a dark violet color, a light blue violet color, a medium blue violet color, a dark blue violet color, a light violet blue color, a medium violet blue color, a dark violet blue color, a violet color, a crimson color, a scarlet color, a burgundy color, a cherry red color, a tomato red color, a fire engine red color, a ruby color, a maroon color, a brick red color, a raspberry color, a vermilion color, a cardinal red color, a rose red color, a blood red color, a salmon red color, or any combination thereof. In some embodiments, a color pattern is or comprises a uniform pattern, a striped pattern, a banded pattern, a sectorial pattern, a variegated pattern, or a gradient pattern. In some embodiments, a color pattern is or comprises a non-uniform pattern, a mottled pattern, a speckled pattern, a spotty pattern, a patchy pattern, or an irregular pattern.

In some embodiments, an engineered plant of the present disclosure, or a part thereof, can be characterized using an imaging modality. In some embodiments, imaging of an engineered plant may comprise microscopic imaging (e.g., for assessment of presence or absence of a pigment associated with one or more colors). In some embodiments, imaging of an engineered plant may comprise spectrophotometry (e.g., using a color model developed by the International Commission on Illumination.

In some embodiments, an engineered plant of the present disclosure, or a part thereof, can be characterized using a biochemical assay. In some embodiments, a biochemical assay may comprise quantification of a pigment associated with one or more colors via high-performance liquid chromatography (HPLC) or liquid chromatography and mass spectrometry (LC-MS). In some embodiments, an association (e.g., a ratio) between two or more pigments may be determined using a biochemical assay. In some embodiments, a pigment is or comprises betalain. In some embodiments, a pigment is or comprises betacyanin. In some embodiments, a pigment is or comprises betaxanthin. In some embodiments, a pigment is or comprises betanin.

In some embodiments, an engineered plant of the present disclosure, or a part thereof, can be characterized using an assay that measures expression of one or more pigment-producing polypeptides (e.g., wherein an engineered plant has been engineered to produce one or more pigment-producing polypeptides).

In some embodiments, expression of a nucleic acid encoding one or more pigment-producing polypeptides may be measured using standard techniques, for example, by determining transcript levels using a microarray, a quantitative polymerase chain reaction, a Northern blot, or a sequencing assay.

In some embodiments, expression of one or more pigment-producing polypeptides may be measured determining protein levels using standard techniques, for example, with a Western blot, liquid chromatography and mass spectrometry, an enzyme-linked immunosorbent assay, or flow cytometry.

### Propagating Plants

In some embodiments, compositions described herein are provided as part of a method of producing a certain compounds (e.g., betanin) in a CPOS, or a method of manipulating, and preferably improving certain properties of a plant, comprising introducing into a plant cell at least one vector as described herein. In some embodiments, a method entails causing or allowing recombination between a vector and the plant cell genome (e.g., nuclear, mitochondrial, and/or chloroplastic genetic material) to introduce at least nucleotide sequence encoding a metabolism modifying gene into the plant genome. It may optionally further comprise the steps of regenerating a plant and cultivating it.

In some embodiments, compositions described herein comprise an engineered plant (e.g., Epipremnum aureum) that has been transformed by Agrobacterium tumefaciens comprising a vector of interest. In some embodiments, a plant (e.g., Epipremnum aureum) is transformed through methods known in the art, for example, as described in Kotsuka & Tada “Genetic transformation of golden pothos (Epipremnum aureum) mediated by Agrobacterium tumefaciens”, Plant Cell Tissue Organ Culture, 2008; which is incorporated herein by reference in its entirety.

In some embodiments, compositions described herein comprise an engineered plant (e.g., Epipremnum aureum) that has been propagated through a traditional method such as “eye cutting”. In some embodiments, an engineered plant (e.g., Epipremnum aureum) is propagated through methods known in the art, for example, as described in UC MASTER GARDENERS NAPA COUNTY “Healthy Garden Tips-Plant Propagation” handbook, published in March 2011 by the University of California and found on the internet at “ucanr.edu/sites/ucmgnapa/files/81929.pdf”; which is incorporated herein by reference in its entirety.

In some embodiments, following transformation, a plant may be regenerated, e.g. from single cells, callus tissue or leaf discs, as is standard in the art. Most plants can be entirely regenerated from cells, tissues and organs of said plant. Available techniques are known in the art and reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

In some embodiments, compositions described herein comprise an engineered plant (e.g., Epipremnum aureum) that has been regenerated from a callus following transformation. In some embodiments, an engineered plant (e.g., Epipremnum aureum) is regenerated through methods known in the art, for example, as described in Zhang, Chen, and Henny “Direct somatic embryogenesis and plant regeneration from leaf, petiole, and stem explants of Golden Pothos” Plant Cell Reports 2005; which is incorporated herein by reference in its entirety.

In some embodiments, microbes are sprayed onto a plant. In some embodiments, plants are dipped into a solution comprising microbes. In some embodiments, microbes are applied to a suitable microbial growth media. In some embodiments, an interior of a container is coated with a composition comprising microbes. In some embodiments, microbes are supplied as a powder and/or liquid to be added to a plant during regular maintenance (e.g., during watering, fertilizing etc.).

In some embodiments, application of a microbe may occur one time, two times, three times, four times, five times, or greater than five times. In some embodiments, microbes are reapplied every 2 weeks, 4 weeks, 6 weeks, 8 weeks, 10 weeks, or 12 weeks. In some embodiments, microbes are reapplied based upon a method of characterizing as described herein. In some embodiments, microbes are reapplied based upon the measurement of culture forming units found in a sample of a plant microbiome when compared to an appropriate control.

### Methods of Manufacturing

In some embodiments, provided herein are methods of manufacturing an engineered plant described herein. In some embodiments, manufacturing an engineered plant comprises introducing to a plant, or a part thereof, a construct described herein. A construct described herein can be introduced to a plant, or a part thereof, using any known method of gene delivery to a plant, including but not limited to, biolistic particle delivery or agrobacterial transformation. Alternatively or additionally, manufacturing an engineered plant of the present disclosure may comprise a standard genetic engineering technique, including but not limited to: CRISPR-Cas mediated gene editing, gene silencing, gene knock-in, or gene knock-out.

In some embodiments, a construct described herein can be introduced to a plant, or a part thereof, using biolistic particle delivery. In some embodiments, biolistic particle delivery comprises loading a construct described herein onto microparticles, and injecting loaded microparticles a plant, or a part thereof, using a gene gun. In some embodiments, loaded microparticles are injected into a plant, or a part thereof, at high pressure using an inert gas (e.g., helium). In some embodiments, microparticles are heavy metal microparticles (e.g., gold microparticles or tungsten microparticles). In some embodiments, a plant part comprises or is an organelle, a cell, a tissue, or an organ. In some embodiments, a plant part comprises or is a tissue. In some embodiments, a plant part comprises or is a callus. In some embodiments, a plant part comprises or is a petiole.

In some embodiments, a construct described herein can be introduced to a plant, or a part thereof, using agrobacterial transformation. In some embodiments, agrobacterial transformation comprises transforming an agrobacterium with a construct described herein. In some embodiments, an agrobacterium comprises a construct described herein. In some embodiments, a construct comprises one or more coding sequences encoding one or more pigment-producing polypeptides. In some embodiments, a agrobacterium comprises a construct comprising a nucleic acid sequence according to SEQ ID NO: 160, 161, 162, or 163. In some embodiments, agrobacterial transformation comprises introducing an agrobacterium comprising a construct described herein to a part of a plant (e.g., thereby transforming a part of a plant) and cultivating or maintaining a transformed part of the plant. In some embodiments, a construct, or a part thereof (e.g., a nucleic acid sequence comprising a coding sequence for one or more pigment-producing polypeptides) is integrated into a genome of a plant. In some embodiments, a construct, or a part thereof (e.g., a nucleic acid sequence comprising a coding sequence for one or more pigment-producing polypeptides) is integrated into a genome of at least one cell in a plant.

In some embodiments, an engineered plant described herein may be manufactured using CRISPR-Cas mediated gene editing (see, for example, Watanabe et al., 2017, Sci Rep., 7:10028).

In some embodiments, an engineered plant described herein may be manufactured via gene knockout (see, for example, Sieburth et al., 1998, Development, 125 (21): 4303-12) or gene knock-in (see, for example, Schreiber et al., 2024, Mol Plant, 17 (5): 824-837). In some embodiments, gene knockout or gene knock-in may be achieved via Cre/loxP recombination. In some embodiments, gene knockout or gene knock-in may be achieved via CRISPR-Cas mediated knockout or knock-in.

In some embodiments, an engineered plant described herein may be manufactured via gene silencing (see, for example, Li et al., 2022, Angew Chem Int Ed Engl, 61 (40): e202210014). In some embodiments, gene silencing may be performed using small interfering ribonucleic acid (siRNA) or short hairpin ribonucleic acid (shRNA).

In some embodiments, an engineered plant described herein can be manufactured using a method described herein.

In some embodiments, an engineered plant, or a part thereof, as described herein exhibits an expression of one or more pigment-producing polypeptides. In some embodiments, a part of a plant is or comprises a petiole. In some embodiments, when cultivated or maintained, a part of a plant is characterized in that it exhibits a pigmentation pattern. In some embodiments, a pigmentation pattern comprises a color or color pattern. In some embodiments, a color is or comprises a pink color, a red color, a purple color, or a brown color. In some embodiments, a color is or comprises a pink color variant, a red color variant, a purple color variant, or a brown color variant. In some embodiments, a color is or comprises a light red pink color, a medium red pink color, a pink color, a light blue pink color, a medium blue pink color, a dark blue pink color, an orange red color, a light red color, a medium red color, a dark red color, a medium purple red color, a dark purple red color, a brown red color, a medium brown purple color, a dark brown purple color, a medium purple color, a dark purple color, a light violet color, a medium violet color, a dark violet color, a light blue violet color, a medium blue violet color, a dark blue violet color, a light violet blue color, a medium violet blue color, a dark violet blue color, a violet color, a crimson color, a scarlet color, a burgundy color, a cherry red color, a tomato red color, a fire engine red color, a ruby color, a maroon color, a brick red color, a raspberry color, a vermilion color, a cardinal red color, a rose red color, a blood red color, a salmon red color, or any combination thereof. In some embodiments, a color pattern is or comprises a uniform pattern, a striped pattern, a banded pattern, a sectorial pattern, a variegated pattern, or a gradient pattern. In some embodiments, a color pattern is or comprises a non-uniform pattern, a mottled pattern, a speckled pattern, a spotty pattern, a patchy pattern, or an irregular pattern.

In some embodiments, manufacturing an engineered plant described herein further comprises retrieving one or more plant parts of an engineered plant comprising a pigmentation pattern, and propagating a different plant (e.g., from said plant part). In some embodiments, manufacturing an engineered plant described herein further comprises retrieving one or more plant parts of an engineered plant comprising one or more pigment-producing polypeptides and propagating a different plant (e.g., from said plant part).

In some embodiments, provided herein are methods of producing an engineered plant described herein. In some embodiments, provided herein are methods of making an engineered plant described herein.

An invention according to any of the following Exemplary Embodiments:
Embodiments 1. An engineered indoor ornamental plant comprising one or more plant parts, wherein the one or more plant parts comprise a color pattern.
Embodiments 2. An indoor ornamental plant engineered to produce one or more plant parts comprising a color pattern associated with expression of one or more heterologous proteins.
Embodiments 3. The indoor ornamental plant of Embodiments 1 or 2, wherein the indoor ornamental plant is Epipremnum aureum.
Embodiments 4. An indoor ornamental plant that produces one or more leaves with a red and/or purple color pattern.
Embodiments 5. An indoor ornamental plant that has leaves comprising a red and/or purple color pattern.
Embodiments 6. An indoor ornamental plant comprising one or more plant parts expressing one or more heterologous proteins associated with a betalain pigment.
Embodiments 7. A plant part comprising a color pattern e.g., associated with expression of one or more heterologous proteins.
Embodiments 8. An isolated plant part comprising a color pattern e.g., associated with expression of one or more heterologous proteins.
Embodiments 9. A plant part comprising a color pattern e.g., associated with expression of one or more heterologous proteins, wherein said plant part is in a culture.
Embodiments 10. An engineered ornamental indoor plant characterized in that:
   (a) one or more heterologous proteins are introduced to the plant, plant cell, or plant part to express betanin; and
   (b) when cultivated or maintained, the plant exhibits a betalain pigment.
Embodiments 11. An ornamental indoor plant engineered to express betalain and characterized by a color pattern that comprises a level of betalain in one or more plant part (e.g., roots (and e.g., optionally in leaves)).
Embodiments 12. The plant of any one of the preceding claims, wherein the plant is characterized by a uniformed color pattern.
Embodiments 13. The plant of any one of the preceding claims, wherein the plant is characterized by a non-uniformed color pattern.
Embodiments 14. The plant of any one of the preceding claims, wherein the plant is characterized by a one or more colors or patterns.
Embodiments 15. The plant of any one of Embodiments 11-14, wherein the color is or comprises a pink, red, purple, or color variant thereof (e.g., pink color variant, red color variant, or purple color variant).
Embodiments 16. The plant of any one of Embodiments 11-15, wherein the color is or comprises a light red pink color, medium red pink color, pink color, light blue pink color, medium blue pink color, dark blue pink color, orange red color, light red color, medium red color, dark red color, medium purple red color, dark purple red color, brown red color, medium brown purple color, dark brown purple color, medium purple color, dark purple color, light violet color, medium violet color, dark violet color, light blue violet color, medium blue violet color, dark blue violet color, light violet blue color, medium violet blue color, dark violet blue color, red color, violet color, crimson color, scarlet color, burgundy color, cherry red color, tomato red color, fire engine red color, ruby color, maroon color, brick red color, raspberry color, vermilion color, cardinal red color, rose red color, blood red color, salmon red color, or a combination thereof.
Embodiments 17. The plant of any one of the preceding claims, wherein a polynucleotide sequences is introduced to the plant to produce said color pattern.
Embodiments 18. The plant of Embodiment 17, wherein the polynucleotide sequence encodes one or more heterologous proteins.
Embodiments 19. The plant of Embodiment 18, wherein the one or more heterologous proteins comprises a one or more CYP76AD1 polypeptide, one or more L-DOPA 4, 5-dioxygenase (DODA) polypeptide, and/or one or more Glucosyl Transferase polypeptide, optionally one or more ADHα polypeptide.
Embodiments 20. The plant of Embodiment 18 or 19, wherein the polynucleotide sequence comprises a promoter.
Embodiments 21. The plant of any one of Embodiments 17-20, wherein the polynucleotide sequence comprises one or more polycistronic elements.
Embodiments 22. The plant of Embodiment 21, wherein the polycistronic element is or comprises a P2A, IRES, or T2A sequences.
Embodiments 23. The plant of Embodiment 22, wherein the polynucleotide sequence encoding the heterologous proteins are operatively linked by the P2A, IRES, or T2A sequence.
Embodiments 24. The plant of any one of Embodiments 17-23, wherein the color pattern is attributable to an expression of the polynucleotide sequence.
Embodiments 25. A method of engineering the plant of any one of Embodiments 1-24 comprising:
   (a) introducing one or more heterologous proteins to a plant, plant cell, or plant part
   (b) when cultivated or maintained, the plant exhibits expression of one or more heterologous proteins;
wherein the engineered plant produces one or more plant parts comprising a color pattern. Embodiments 26. A method of assessing the engineered plant of any one of Embodiments 1-24 comprising:
   (a) cultivating or maintaining said engineered plant, and
   (b) determining a level of expression of one or more heterologous proteins.
Embodiments 27. A method of assessing the engineered plant of any one of Embodiments 1-24 comprising:
   (a) cultivating or maintaining said engineered plant,
   (b) determining a level of expression of one or more heterologous proteins, and
   (c) comparing the level of expression of one or more heterologous proteins to a reference (e.g., plant that has not been engineered, e.g., parental un-modified plant).
Embodiments 28. A propagating the engineered plant of any one of Embodiments 1-24 comprising:
   (a) introducing one or more heterologous proteins to a plant, plant cell, or plant part
   (b) when cultivated or maintained, the plant exhibits expression of one or more heterologous proteins; wherein the engineered plant produces one or more plant parts comprising a color pattern; and
   (c) retrieving one or more plant parts comprising the one or more heterologous proteins sufficient to propagate a different plant comprising the color pattern.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

### Example 1: Creation, Isolation, and Formulation of Vectors for Plant and/or Microbe Transformation

This example provides information regarding the creation, isolation, and formulation of vectors for plant and/or microbe transformation.

Genetic manipulation techniques were performed using technologies known in the art (e.g. Golden Gate cloning systems) and according to manufacturer's instructions. Genes were cloned from appropriate genomic DNA sources isolated using standard protocols such as miniprep or midiprep. The correct sequence of genes of interest were characterized using PCR followed by restriction enzyme digestion and gel electrophoresis and/or by PCR followed by Sanger Sequencing.

### Example 2: Modification of an Indoor Ornamental Plant

This example relates to the transformation of an exemplary indoor ornamental plant (e.g., Epipremnum Aureum) with vectors comprising sequences described herein. Exemplary methods are described herein.

### 1-Agrobacterium-Mediated Transformation:

1-1: Preparing material for transformation: young stem and petioles from young Epipremnum aureum were surface-sterilized with a sodium hypochlorite solution (2% chlorine) and a drop of Tween 20 for 25 min with agitation. Explants were then rinsed three times with sterile distilled water and cut into 0.5-1 cm long segments on MS medium (Murashige and Skoog 1962) supplemented with 2.0 mg 1-1N-phenyl-NO-1,2,3-thiadiazol5-yl urea (TDZ), 0.2 mg 1-1 a-naphthalene acetic acid (NAA), 3% sucrose, and 7 gr/L agar and adjusted to pH 5.8 (referred to herein as regeneration media (RM)).
1-2: Agrobacterium preparation for the transformation of golden Epipremnum aureum: A. tumefaciens strain EHA105 containing a plasmid of interest was used for the transformation of golden Epipremnum aureum. The A. tumefaciens strain was grown in 5 ml of LB liquid medium supplemented with 50 mg/L spectinomycin and 30 mg/L rifamycin at 30 C until the absorbance at 600 nm reached 0.8-1.0. The strain was then transformed with a plasmid of interest. Plasmids used for transformation comprised a selection marker (e.g., hygromycin phosphotransferase gene driven by the 35S promoter). Following transformation, 25 mg/L hygromycin B was used as a selection agent in the regeneration media.
1-3: Infection and Transformation: pre-cultured Epipremnum aureum stem explants were immersed for 20 minutes in an A. tumefaciens suspension with liquid medium (RM media without agar) supplemented with 0.1 mM acetosyringone, explants were occasional agitated to ensure exposure to A. tumefaciens.
1-4: Co-Incubation: explants were then transferred onto an RM co-incubated media plate and stored for three days in a dark growth chamber at 26° C.
1-5: Selection and embryogenesis: after co-cultivation, explants were rinsed three times with liquid medium, comprising 100 mg/L cefotaxime, 100 mg/L carbenicillin, and 30 mg/L hygromycin. Explants were then returned to a dark growth chamber kept at 26° C. Explants were transferred to fresh medium (RM) every 2-3 weeks to avoid oxidative products released from the hygromycin, these products can induce undesirable necrotic browning tissues. Embryogenic calli were readily observed after approximately 8-12 weeks of culture.
1-6: Shoot generation: hygromycin-resistant embryos were transferred onto germination medium comprising MS-medium supplemented with 0.2 mg 1-1 NAA, 2 mg 1-1 6-benzylaminopurine (BAP), 3% sucrose, and 0.7% Agar (pH 5.8).
1-7: Root generation and transfer to soil: germinated shoots were then transferred onto an MS medium supplemented with 1% sucrose (pH 5.8) in plant boxes for further growth of shoots and roots. Grown plants were transferred to soil to propagate under standard greenhouse conditions with a 16 h/8 h photoperiod at 25°/20° C. day/night, and 60% relative humidity.

### Example 3: Demonstration of Heterologous Gene Expression in an Indoor Ornamental Plant

This example relates to the confirmation of heterologous gene expression in transformed an exemplary indoor ornamental plant, (e.g., Epipremnum aureum). Certain methods to confirm transgene introduction into Epipremnum aureum are described herein.

Approximately 20-30 mg of transformed leaf pieces were collected and placed in a 1.5 mL Eppendorf tube containing 2 stainless steel beads of 3 mm diameter. The tube was then flash frozen in liquid nitrogen and introduced into a mixer mill (Retsch MM400) to lyse the samples (shaking at 30 Hz for 1 minute). Following lysis, 500 μL of GEx buffer was added (5.5 M Guanidine Thiocyanate, 20 nM Tris-HCl, pH 6.6) and the sample was vortexed vigorously. The samples were centrifuged for 5 minutes at 20,000 g and the supernatant was loaded on a Silica Membrane Mini Spin Column (from any DNA purification kit). The column with the sample was centrifuged at 20,000 g for 1 minute and the membranes were washed twice with 750 μL of cleaning buffer (80% ethanol, 10 mM Tris-HCl, pH 7.5). To remove any trace of ethanol, the samples were centrifuged at 20,000×g for 1 min and the genomic DNA was eluted by adding 50 μL of ddH2O to the column followed by centrifugation at 20,000×g for 1 min. The extracted genomic DNA was used in a PCR with primers specific to the transgene of interest to confirm transgenesis.

PCR was conducted as known in the art. In brief, PCR conditions were as follow: in a 25 μL total reaction volume, 1 μL of DNA, 2.5 μL of 10× FastStart buffer with MgCl2 (Roche), 0.5 μL of 10 mM dNTP (Roche), 2.5 μL of forward primer at 10 mM, 2.5 μL of reverse primer at 10 mM, 0.2 μL of FastStart Taq (Roche, Cat. No. 12 032 937 001) and 15.8 μL of ddH2O. The cycling conditions of the PCR were optimized for each primer pair, but in general were as follows: 95° C. for 4 minutes, 35 cycles of: 95° C. for 30 seconds 55° C. for 30 and seconds 72° C. for 1 minute, 72° C. for 5 minutes, and hold at 12° C. The PCR products were analyzed on a 2.5% agarose gel stained with BET and the fragments size was compared to the known theoretical size using a DNA ladder as reference.

When a Epipremnum aureum plant was confirmed to have integrated a transgene, the transgenes expression level is tested and confirmed by qPCR. Briefly: a leaf sample of 100 mg was taken and placed in a 1.5 mL Eppendorf tube containing 2 stainless steel beads of 3 mm diameter. The tube was then flash frozen in liquid nitrogen and introduced into a mixer mill (Retsch MM400) to lyse the samples (shaking at 30 Hz for 1 minute). RNA extraction was then performed with the Macherey Nagel NucleoSpin RNA Plant, Mini kit for RNA from plant, ref: 740949.50 (according to the manufacturer instructions). Once RNA was purified, qPCR reactions were set up using the NEB Luna® Universal One-Step RT-qPCR Kit (Ref: E3005L). In a 5 μL total reaction volume, 2.5 μL of Luna Universal One-Step Reaction Mix (2×), 0.5 μL of Luna WarmStart® RT Enzyme Mix (20×), 0.2 μL of forward primer at 10 mM, 0.2 μL of reverse primer at 10 mM, 1 μL of RNA and 0.85 μL of nuclease-free water. Primer efficiency was tested using serial dilutions of the RNA (1 to 10,000 fold), all reactions were performed in at least triplicate. For each RNA sample, a Epipremnum aureum endogenous gene (actin) was used as the reference for calculating expression levels. The reaction was run on a LightCycler® 96 from Roche.

A skilled practitioner of the art will recognize that DNA and RNA extraction protocols, and PCR and qPCR reaction protocols can vary greatly while still producing valuable and informative data.

### Example 4. Exemplary Synthesis of a Pigment Associated with a Color Pattern in an Engineered Indoor Ornamental Plant

This example relates to biosynthesis of a pigment (e.g., betalain or its derivatives, e.g., betanin) associated with a color and/or pattern in an engineered indoor ornamental plant.

Modification of a plant (e.g., an indoor ornamental plant) with genetic elements described herein can be particularly useful in producing an engineered plant with a color and/or pattern associated with biosynthesis of a pigment (e.g., betalain or its derivatives, e.g., betanin). Modification of genetic elements described herein can further enhance biosynthesis of a pigment (e.g., betalain or its derivatives, e.g., betanin) associated with a color and/or pattern of an engineered indoor ornamental plant.

### Example 5: Assessment of Pigment Associated with a Color Pattern in an Engineered Indoor Ornamental Plant

This example relates to measuring a pigment associated with a color and/or pattern in an exemplary indoor ornamental plant engineered. In some embodiments, an engineered plant described herein can have a different (e.g., not green) color pattern when compared to a reference (e.g., non-engineered plant).

Exemplary methods described herein can assess a level of a pigment (e.g., betanin) associated with a color and/or pattern.

Pigment can be extracted from the collected sample by adding 2 bids into each tube and shaken with a TissueLyser at 30 Hz for 20 seconds. Fresh plant tissue is then weighed out in 2 mL Eppendorf tubes to reach 20 mg, and a mixture of 250 μL of methanol, 250 μL of water and 500 μL of chloroform is added to the tubes with the fresh plant tissue. The mixture is then shaken with a TissueLyser at 30 Hz for 1 minute, and mixed with a Thermomix at 4° C. for 30 min. Supernatant is obtained via centrifugation 4° C. for 30 min, and 50 μL of the supernatant is transferred to high-performance liquid chromatography (HPLC) vials for liquid chromatography-mass spectrometry (LC-MS) analysis. HPLC is performed with a Shimadzu UHPLC Nexera Series XS 1050 Bar apparatus. Mobile Phase A, consisting of a mixture of water and 0.1% formic acid is mixed with Mobile Phase B, consisting of a mixture of acetonitrile and 0.1% formic acid. The supernatant is mixed with the Mobile Phase mixture, and 4 μL of the final mixture are then carried through the Discovery HS F5-3 15 cm×2.1 mm packed column (Solid Phase), which is kept at 30° C. The sample is then detected by the mass spectrometer, device model LCMS-8045. The mass spectrometer is kept at the following parameters: 3 L/min Nebulizing Gas Flow, 10 L/min Heating Gas Flow, Interface temperature of 300° C., Dissolution temperature of 526° C., DL temperature of 250° C., Heat Block temperature of 400° C., and 10 L/min Drying Gas Flow. Betanin levels in mg/g can then be quantified via multiple reaction monitoring (MRM) using the following fragmentation of betanin from the precursor ion to the product ion: 551.1 m/z→389.2 m/z.

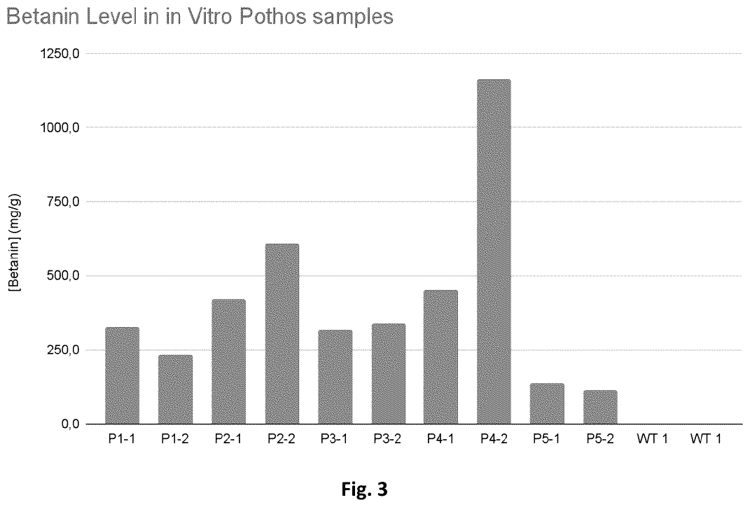

The present example demonstrates that engineered indoor ornamental plants, as shown in FIG. 3, exhibit an increase in a level of betanin as compared to wild-type plants. Two plants samples were collected from each tested plant (P1, P2, P3, P4, P5, and WT). FIG. 3 shows that all engineered ornamental indoor plants produce betanin at a concentration of at least about 100 mg/g, whereas wild-type plants exhibit no (e.g., 0 mg/g) production of betanin.

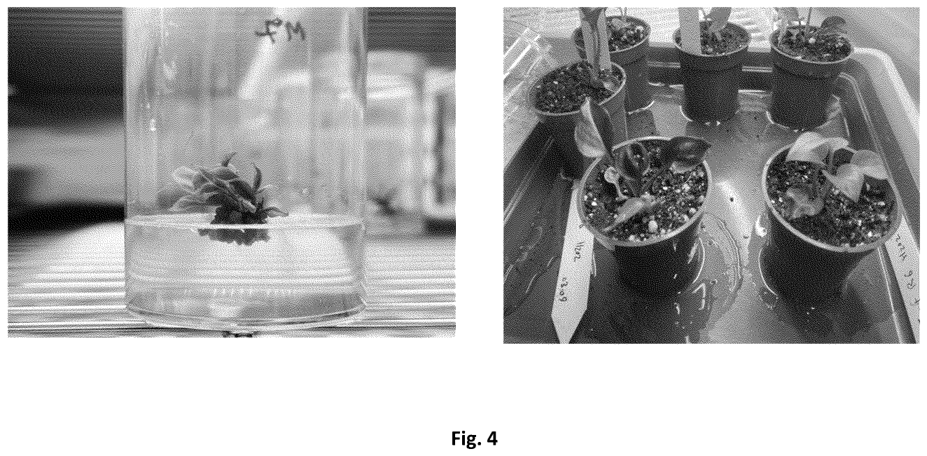

As demonstrated in FIG. 4, engineered plants as described herein can exhibit a red-color pattern.

In some embodiments, engineered plants as described herein may produce betanin at a level of about 100 mg/gram of fresh weight to about 1200 mg/gram of fresh weight.

### Example 6: Exemplary Characterization of an Engineered Indoor Ornamental Plant

This example relates to characterization of an exemplary engineered indoor ornamental plant by quantifying a level of a pigment (e.g., betanin) associated with a color and/or pattern relative to a pigment (e.g., chlorophyll a) not associated with said color and/or pattern. In some embodiments, an engineered plant described herein can have a different (e.g., not green) color pattern when compared to a reference (e.g., non-engineered plant).

### Materials and Methods

Wild-type indoor ornamental plants of species Epipremnum aureum were transformed with methods as described in Example 2. Exemplary Epipremnum aureum plants were transformed with constructs as described in Table 18.

TABLE 18
Exemplary constructs tested in Epipremnum aureum plants.

| | | | Observed | |
|---|---|---|---|---|
| | | | Expression | |
| | | SEQ | of Coding | Pigmentation |
| Construct | Description | ID NO: | Sequence | Phenotype |
| 1 | PL-888 | 160 | YES | YES |
| 2 | PL-1044 | 161 | YES | YES |
| 3 | PL-418 | 189 | YES | NO |
| 4 | PL-450 | 190 | YES | NO |
| 5 | PL-451 | 191 | YES | NO |
| 6 | PL-454 | 192 | YES | NO |
| 7 | PL-455 | 193 | YES | NO |
| 8 | PL-456 | 194 | YES | NO |
| 9 | PL-461 | 195 | YES | NO |
| 10 | PL-462 | 196 | YES | NO |
| 11 | PL-463 | 197 | YES | NO |
| 12 | PL-464 | 198 | YES | NO |
| 13 | PL-465 | 199 | YES | NO |

Betanin and chlorophyll a levels were quantified as follows. Samples of an engineered indoor ornamental plant Epipremnum aureum were obtained by removing a fresh leaf from the plant, and frozen in liquid nitrogen in 5 mL EPPENDORF™ tubes. A flash-frozen leaf was powdered in a mortar with a pestle, 20 mg of homogenized leaf powder were weighed, mixed with magnetic beads and finely homogenized using a QIAGEN™ tissue lyser. A mixture of 250 μL of methanol, 250 μL of water and 500 μL of chloroform was added to EPPENDORF™ tubes with the homogenized plant tissue, shaken with a TissueLyser at 30 Hz for 30 s, and mixed with a Thermomix at 4° C. for 30 min. Supernatant was obtained via centrifugation at 13500 rpm for 10 min at 4° C., and 50 μL of supernatant was transferred to high-performance liquid chromatography (HPLC) columns, and for liquid chromatography-mass spectrometry (LC-MS) analysis was performed with a Shimadzu LC-FLD-MS 8045 apparatus. Betanin levels in mg/g were quantified via multiple reaction monitoring (MRM) using the following fragmentation of betanin from the precursor ion to the product ion: 551.3 m/z→389.2 m/z.

Another 50 μL of supernatant were collected and transferred to Discovery HS F5-3 HPLC columns for analysis. Shimadzu LC-FLD-MS 8045 apparatus equipped with a fluorescence detector was used to quantify emission of light from chlorophyll a. Excitation wavelength was set to 430 nm, and emission wavelength was set to 630 nm.

### Results

Exemplary Epipremnum aureum plants transformed with constructs 1 and 2 from Table 18 exhibited expression of a corresponding construct, and a pigmentation phenotype, where the pigmentation pattern comprised a pink color or a red color. Expression of transformed coding sequences and pigmentation phenotype were assessed in stems and leaves of plants transformed with constructs 1 and 2. Exemplary Epipremnum aureum plants transformed with constructs 3-13 from Table 18 demonstrated expression of a corresponding construct, but did not exhibit a pigmentation phenotype, where the pigmentation pattern comprised a pink color or a red color.

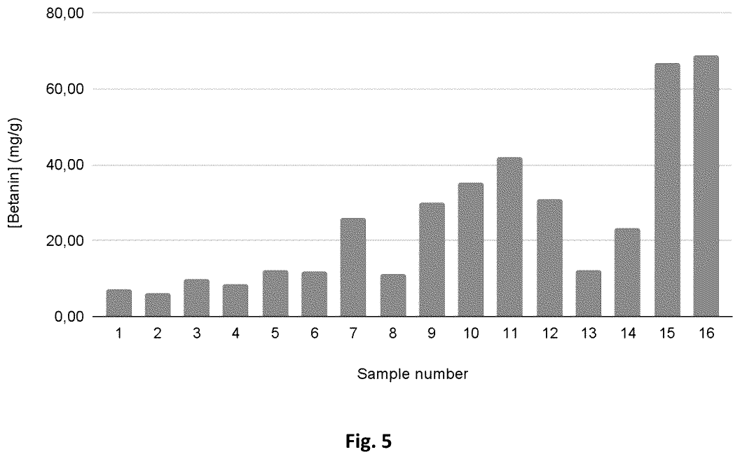

Betanin levels in indoor ornamental plants transformed with constructs 1 and 2 from Table 18 were quantified, and ranged between about 5 mg/g to about 70 mg/g, as shown in FIG. 5. In these plants, the ratio of betanin concentration to chlorophyll a concentration ranged between about 18 to about 172, as shown in FIG. 5.

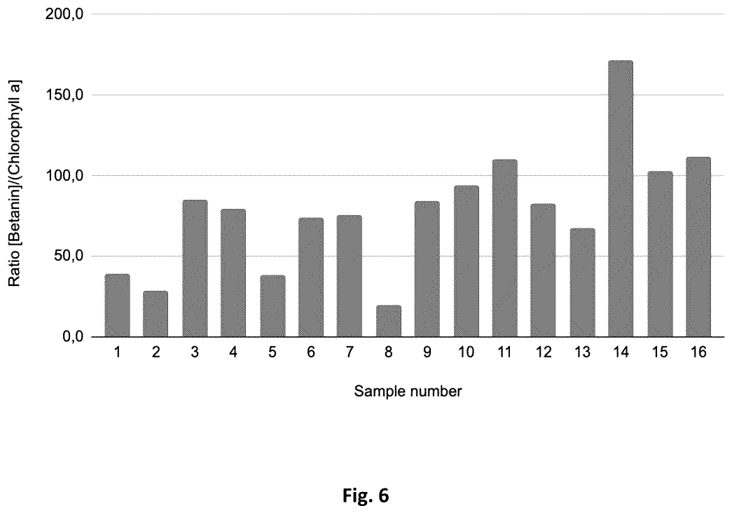

In visibly red engineered plants 7, 8 9, and 10, corresponding to samples 13, 14, 15, and 16, betanin concentration was about 12, about 23, about 65, and about 70 mg/g, respectively (FIG. 5). In the same samples, the betanin-to-chlorophyll a ratio was about 66, about 172, about 102, and about 111, respectively (FIG. 6).

Engineered plants 1 (samples 1 and 2), 2 (samples 3 and 4), 5 (samples 9 and 10), and 6 (samples 11 and 12) exhibited pink color and/or pattern. Concentration of betanin in samples 1 and 2 was about 7 mg/g and about 6 mg/g, respectively (FIG. 5), and the betanin-to-chlorophyll a ratio was about 39 and 28 (FIG. 6). Concentration of betanin in samples 3 and 4 was about 10 mg/g and about 9 mg/g, respectively (FIG. 5), and the betanin-to-chlorophyll a ratio was about 84 and 79 (FIG. 6). Concentration of betanin in samples 9 and 10 was about 30 mg/g and about 36 mg/g, respectively (FIG. 5), and the betanin-to-chlorophyll a ratio was about 84 and 94 (FIG. 6). Concentration of betanin in samples 11 and 12 was about 42 mg/g and about 32 mg/g (FIG. 5), respectively, and the betanin-to-chlorophyll a ratio was about 110 and 83 (FIG. 6).

The present example demonstrates that exemplary engineered indoor ornamental plants produce betanin and exhibit an association of an increasing betanin-to-chlorophyll ratio with an increasing observed red color and/or pattern.

1-134. (canceled)

135. A plant engineered to express:
(i) a heterologous CYP76AD1 polypeptide,
(ii) a heterologous L-DOPA 4,5-dioxygenase (DODA) polypeptide,
(iii) a heterologous cyclo-DOPA 5-O-glucosyltransferase (cDOPA5GT) polypeptide, or
(iv) any combination of (i)-(iii).

136. The plant of claim 135, wherein the plant is further engineered to express:
(i) a heterologous arogenate dehydrogenase (ADH) polypeptide,
(ii) a heterologous 3-deoxy-D-arabino-heptulosonate 7-phosphate synthase (DHAP synthase) polypeptide, or
(iii) both of (i) and (ii).

137. The plant of claim 135, wherein:
(i) the heterologous CYP76AD1 polypeptide has a sequence with at least 80% identity to SEQ ID NO: 12,
(ii) the heterologous DODA polypeptide has a sequence with at least 80% identity to SEQ ID NO: 13, and
(iii) the heterologous cDOPA5GT polypeptide has a sequence with at least 80% identity to SEQ ID NO: 11.

138. The plant of claim 136, wherein:
(i) the heterologous ADH polypeptide has a sequence with at least 80% identity to SEQ ID NO: 8, and
(ii) the heterologous DHAP synthase polypeptide has a sequence with at least 80% identity to SEQ ID NO: 133.

139. The plant of claim 135, wherein the plant comprises:
(i) a nucleic acid encoding a heterologous CYP76AD1 polypeptide, wherein the nucleic acid has a sequence with at least 80% identity to SEQ ID NO: 127,
(ii) a nucleic acid encoding a heterologous DODA polypeptide and having a sequence with at least 80% identity to SEQ ID NO: 2,
(iii) a nucleic acid encoding a heterologous cDOPA5GT polypeptide, wherein the nucleic acid has a sequence with at least 80% identity to SEQ ID NO: 128, or
(iv) any combination of (i)-(iii).

140. The plant of claim 139, wherein the plant further comprises:
(i) a nucleic acid encoding a heterologous ADH polypeptide, wherein the nucleic acid has a sequence with at least 80% identity to SEQ ID NO: 129,
(ii) a nucleic acid encoding a heterologous DHAP synthase polypeptide, wherein the nucleic acid has a sequence with at least 80% identity to SEQ ID NO: 132, or
(iii) any combination of (i)-(ii).

141. The plant of claim 139, wherein the plant comprises a nucleic acid comprising:
(i) a nucleic acid comprising one or more promoters selected from: PvUbi2, ZmUbi, BdUbi10, CsVMV, and 2×35S+TMV 5′UTR;
(ii) a nucleic acid comprising one or more terminators selected from a 3′ UTR+TerCaMV35S terminator, a TerOcs terminator, a TerMas terminator, a NtExt3 terminator, a AtHSP18.2 terminator, and a TerNos terminator;
(iii) a nucleic acid comprising one or more 5′ or 3′ untranslated regions;
(iv) a nucleic acid encoding one or more P2A polypeptides;
(v) a nucleic acid encoding one or more PeaChlSP signal peptides; or
(vi) any combination of (i)-(v).

142. The plant of claim 140, wherein the plant comprises a nucleic acid comprising:
(i) a nucleic acid comprising one or more promoters selected from: PvUbi2, ZmUbi, BdUbi10, CsVMV, and 2×35S+TMV 5′UTR;
(ii) a nucleic acid comprising one or more terminators selected from a 3′ UTR+TerCaMV35S terminator, a TerOcs terminator, a TerMas terminator, a NtExt3 terminator, a AtHSP18.2 terminator, and a TerNos terminator;
(iii) a nucleic acid comprising one or more 5′ or 3′ untranslated regions;
(iv) a nucleic acid encoding one or more P2A polypeptides;
(v) a nucleic acid encoding one or more PeaChlSP signal peptides; or
(vi) any combination of (i)-(v).

143. The plant of claim 135, wherein the plant has been engineered to produce one or more pigments in at least one plant part.

144. The plant of claim 135, wherein the one or more pigments comprise or are a betalain.

145. The plant of claim 144, wherein the at least one plant part comprises a leaf, a node, a stem, and/or a root.

146. The plant of claim 143, wherein the plant has been engineered to comprise a color or a color pattern.

147. The plant of claim 146, wherein the color is or comprises a pink color, a red color, a purple color, a brown color, or any combination thereof.

148. The plant of claim 146, wherein the color pattern is or comprises a non-uniform pattern, a mottled pattern, a speckled pattern, a spotty pattern, a patchy pattern, or an irregular pattern.

149. The plant of claim 146, wherein the color pattern is or comprises a uniform pattern, a striped pattern, a banded pattern, a sectorial pattern, a variegated pattern, or a gradient pattern.

150. The plant of claim 135, wherein the plant is an engineered Epipremnum aureum.

151. The plant of claim 135, wherein the plant is an engineered plant of the family Araceae.

152. A method of making, producing, or manufacturing a plant engineered to express one or more pigment-producing polypeptides, wherein the method comprises introducing into the plant one or more agrobacteria comprising one or more nucleic acids encoding the one or more pigment-producing polypeptides.
